(19) United States
(12) Patent Application Publication
(10) Pub. No.: US 2026/0137771 A1
(43) Pub. Date: May 21, 2026

(54) DEVELOPMENT OF MULTI-ANTIGEN MRNA VACCINE AGAINST FELINE FIPV

(71) Applicants: BEIJING SYNGENTECH CO., LTD., Beijing (CN); JINYU BAOLING BIOPHARMACEUTICAL CO., LTD., Hohhot (CN)

(72) Inventors: Gan Liu, Beijing (CN); Jie Li, Hohhot (CN); Weixi Liao, Beijing (CN); Jinxian Wu, Hohhot (CN); Qiang Liu, Beijing (CN); Cuicui Li, Beijing (CN); Huiya Huang, Beijing (CN); Chen Wang, Hohhot (CN); Pingping Zhao, Hohhot (CN); Xinying Jiang, Hohhot (CN); Yifang Zhao, Hohhot (CN)

(73) Assignees: BEIJING SYNGENTECH CO., LTD., Beijing (CN); JINYU BAOLING BIOPHARMACEUTICAL CO., LTD., Hohhot (CN)

(21) Appl. No.: 19/393,490

(22) Filed: Nov. 18, 2025

Related U.S. Application Data
(63) Continuation of application No. PCT/CN2024/077001, filed on Feb. 8, 2024.

(30) Foreign Application Priority Data
May 18, 2023 (CN) 202310565416.7

(51) Int. Cl. A61K 39/215; A61K 39/00; A61P 37/04

(52) U.S. Cl. CPC A61K 39/215; A61P 37/04; A61K 2039/53; A61K 2039/6018; C12N 2770/20022; C12N 2770/20034; C12N 2770/20071

(57) ABSTRACT

This disclosure relates to the development of a multi-antigen mRNA vaccine against feline infectious peritonitis virus (FIPV). Provided is a pharmaceutical formulation. The pharmaceutical formulation includes a nucleic acid fragment. The nucleic acid fragment is mRNA and includes at least one of a first nucleic acid fragment, a second nucleic acid fragment, or a third nucleic acid fragment. The first nucleic acid fragment encodes an M protein of feline infectious peritonitis virus; the second nucleic acid fragment encodes an N protein of feline infectious peritonitis virus; the third nucleic acid fragment encodes an S protein, an S_ec protein, or an SII protein of feline infectious peritonitis virus; and the first nucleic acid fragment, the second nucleic acid fragment, and the third nucleic acid fragment are linked or not linked.

### CROSS-REFERENCE TO RELATED APPLICATIONS

The present disclosure is a continuation-in-part of International Patent Application No. PCT/CN2024/077001 filed on Feb. 8, 2024, which claims priority to and benefits of patent application No. 202310565416.7, filed with China National Intellectual Property Administration on May 18, 2023, the entire contents of which are incorporated herein by reference.

### STATEMENT REGARDING SEQUENCE LISTING

A Sequence Listing associated with this application is being filed concurrently herewith in ASCII format and is hereby incorporated by reference into the present specification. The text file containing the Sequence listing is titled “Sequence_Listing.xml”, was created on Nov. 10, 2025, and is 522,655 bytes in size.

### FIELD

The present disclosure relates to the field of biotechnology, and specifically, to development of a multi-antigen mRNA vaccine against feline FIPV, and more specifically, to a pharmaceutical formulation, an isolated nucleic acid molecule, an expression vector, a recombinant virus, a liposome, a vaccine, a recombinant cell, a method for constructing a feline infectious peritonitis virus vaccine and a use, and a method for preventing or treating a disease caused by feline infectious peritonitis virus infection.

### BACKGROUND

Feline coronavirus (FCoV) belongs to the Coronaviridae family. The viruses in the Coronaviridae family are characterized by being relatively large, round, enveloped, positive-strand RNA viruses with genomes ranging from 27 kb to 32 kb, which encode the replication polymerase, four structural proteins (S, M, N, and E proteins), and several non-structural proteins. The spike protein (S protein) is one of the important structural proteins of coronaviruses, which is responsible for forming the surface protrusions of these viruses and is also one of the key factors for viral infection. The S protein can bind to receptors on host cells, facilitating the virus to entry the interior of host cells to initiate infection. In addition, the S protein is also an important antigen in many coronavirus vaccines. The SII region of the S protein contains fewer epitopes that mediate antibody-dependent enhancement (ADE) of the immune response. The membrane protein (M protein) is involved in the formation of the particle morphology and the localization of coronaviruses. It is a protein that spans the viral envelope and can interact with other proteins to form the structure of the virion. The nucleocapsid protein (N protein) encapsulates the RNA genome of the virus and is involved in the processes of viral gene replication and transcription. In addition, the N protein can induce a response of the host immune system against the virus. The envelope protein (E protein) is a protein on the envelope of coronaviruses, can interact with the M protein to form the structure of the virion, and is also involved in the processes of viral infection and assembly.

Feline coronavirus (FCoV) can be classified into feline enteric coronavirus (FECV) and feline infectious peritonitis virus (FIPV) according to its biotype or pathogenicity. FECV is highly prevalent, infecting intestinal epithelial cells, and causing no symptoms or only mild diarrhea. FIPV primarily infects feline monocytes and macrophages. It is difficult to distinguish FECV from FIPV based on genomic sequences. Although some studies have suggested that FIPV can be distinguished from FECV by amino acid mutations in the spike protein, these mutations have later been found to be more associated with tissue tropism.

FIPV is typically characterized by the occurrence of pyogranulomatous lesions in various tissues and organs, including the lung, liver, spleen, omentum, and brain. The infection of macrophages and monocytes is considered a key part of the pathogenic mechanism. In the terminal stage of FIPV infection, a significant reduction in T cells in peripheral and lymphoid tissues can be observed, coinciding with the frequent occurrence of hypergammaglobulinemia, which indicates the presence of a severe virus-induced immune dysregulation. Humoral immunity appears to have no protective effect and may lead to “early death syndrome.” When S antibodies are present at sub-neutralizing titers, they can enhance infection of target cells by binding to Fc receptors. Researchers have made multiple attempts to develop the FIPV vaccines based on humoral immunity, but most of these attempts have failed. The main reason for the failure resides in the occurrence of antibody-dependent enhancement (ADE) of infection, which prevents antibodies from exerting an effective protective effect. Currently, researchers are attempting to control FIPV infection and clearance through cell-mediated immunity (CMI), but have not yet achieved satisfactory protective effects.

Therefore, there is an urgent need in the art to develop a vaccine against FIPV

### SUMMARY

The present disclosure is provided by the inventors based on the discovery of the following issues and facts.

At present, there have been no new breakthroughs in the research and development of FIPV vaccines over an extended period, which results in almost all FIPV infections ending in the death of cats.

To this end, in a first aspect of the present disclosure, the present disclosure provides a pharmaceutical formulation. According to an embodiment of the present disclosure, the pharmaceutical formulation includes a nucleic acid fragment. The nucleic acid fragment is mRNA and includes at least one of a first nucleic acid fragment, a second nucleic acid fragment, and a third nucleic acid fragment. The first nucleic acid fragment encodes an M protein of feline infectious peritonitis virus; the second nucleic acid fragment encodes an N protein of feline infectious peritonitis virus; the third nucleic acid fragment encodes an S protein, Sec protein (S extracellular fragment), or SII protein of feline infectious peritonitis virus; and the first nucleic acid fragment, the second nucleic acid fragment, and the third nucleic acid fragment are linked or not linked. According to an embodiment of the present disclosure, the pharmaceutical formulation, expressing any one or combinations of the M protein, the N protein, the S protein, the Sec protein, or the SII protein of feline infectious peritonitis virus can stimulate the cell-mediated immune response in animals.

It should be noted that, in the present disclosure, the M protein, the N protein, the S protein, the S_ec protein, or the SII protein of the wild-type FIPV can also be adaptively modified as desired to reduce the virulence of FIPV, while not affecting its three-dimensional structure and retaining its immunogenicity, so as to prepare a new type of FIPV vaccine. According to the sequence alignment results (Table 1 and Table 2), the M protein has an amino acid sequence with at least 89% homology to SEQ ID NO: 1, and the nucleic acid fragment encoding the M protein has a nucleotide sequence with at least 67% homology to a sequence as set forth in any one of SEQ ID NO: 4 to SEQ ID NO: 7; and the N protein has an amino acid sequence with at least 91% homology to SEQ ID NO: 2, and the nucleic acid fragment encoding the N protein has a nucleotide sequence with at least 70% homology to a sequence as set forth in any one of SEQ ID NO: 8 to SEQ ID NO: 11. The S protein has an amino acid sequence with at least 45% homology to SEQ ID NO: 3, and the nucleic acid fragment encoding the S protein has a nucleotide sequence with at least 51% identity to a sequence as set forth in any one of SEQ ID NO: 12 to SEQ ID NO: 15; the S_ec protein has an amino acid sequence with at least 43% homology to amino acids at sites 1 to 1374 of SEQ ID NO: 3, and the nucleic acid fragment encoding the S_ec protein has a nucleotide sequence with at least 51% identity to nucleotides at sites 1 to 4122 of a sequence as set forth in any one of SEQ ID NO: 12 to SEQ ID NO: 15; and the SII protein has an amino acid sequence with at least 6200 homology to amino acids at sites 782 to 1433 of SEQ ID NO: 3, and the nucleic acid fragment encoding the SII protein has a nucleotide sequence with at least 570 identity to nucleotides at sites 2344 to 4299 of a sequence as set forth in any of SEQ ID NO: 12 to SEQ ID NO: 15. The FIPV vaccine is not particularly limited, as long as it can produce the receptor binding domains of the modified M protein, N protein, S protein, S_ec protein, or SII protein of FIPV in the living organism, has immunogenicity and can induce an immune response in the animal. In addition, the pharmaceutical formulation can be used to stimulate an immune response in all animals that can be infected with feline infectious peritonitis virus, including but not limited to a cat.

TABLE 1
Protein Sequence Homology of Multiple Sub-strains (%)

| | | Type I | | | | | Type II | | | |
|---|---|---|---|---|---|---|---|---|---|---|
| SEQ | | | | | HF | | 79- | SH | Genbank: | 2-C11 |
| ID | Description | KU-2 | UCD1 | Black | 1902 | QS | 1146 | 2211 | GQ152141 | Re 10276 |
| 1 | M | — | 94.3 | 94.7 | 94.7 | 100.0 | 96.3 | 96.3 | 88.6 | 91.7 |
| 2 | N | 92.0 | 91.8 | 91.8 | 91.8 | | 91.8 | 91.5 | 91.5 | 92.8 |
| 3 | S | 45.0 | 45.1 | 44.7 | 48.1 | 45.0 | 100.0 | 99.3 | 94.8 | 93.0 |
| 4 | S_ec | 43.2 | 43.1 | 42.7 | 46.2 | 43.2 | | 99.3 | 94.7 | 93.2 |
| | (extracellular | | | | | | | | | |
| | fragment, | | | | | | | | | |
| | amino acids at | | | | | | | | | |
| | sites 1 to 1374 | | | | | | | | | |
| | of S protein) | | | | | | | | | |
| 5 | SII (amino | 63.2 | 63.3 | 62.4 | 63.6 | 63.0 | | 99.4 | 98.0 | 95.2 |
| | acids at sites | | | | | | | | | |
| | 782 to 1433 of | | | | | | | | | |
| | S protein) | | | | | | | | | |

Note:
— indicates no data

TABLE 2
Nucleic Acid Sequence Homology of Multiple Sub-strains (%)

| | | Type I | | | | | Type II | | | |
|---|---|---|---|---|---|---|---|---|---|---|
| SEQ | | | | | HF | | 79- | SH | Genbank: | 2-C11 |
| ID | Description | KU-2 | UCD1 | Black | 1902 | QS | 1146 | 2211 | GQ152141 | Re 10276 |
| 4 | M wild type | — | 91.7 | 91.7 | 91.7 | 100.0 | 93.3 | 93.2 | 84.8 | 90.7 |
| 5 | M2 | — | 72.4 | 71.1 | 71.1 | | 72.2 | 72.2 | 69.0 | 71.6 |
| 6 | M3 | — | 71.0 | 70.6 | 70.6 | | 70.6 | 70.6 | 67.5 | 70.4 |
| 7 | M6 | — | 73.1 | 72.1 | 72.1 | | 73.2 | 73.5 | 69.6 | 72.5 |
| 8 | N wild type | 91.9 | 91.3 | 91.0 | 91.0 | | 90.7 | 90.3 | 90.8 | 91.3 |
| 9 | N2 | 70.7 | 70.9 | 70.7 | 70.7 | | 70.8 | 70.7 | 70.5 | 70.7 |
| 10 | N3 | 71.8 | 71.8 | 71.7 | 71.7 | | 72.3 | 72.1 | 71.6 | 71.3 |
| 11 | N6 | 73.8 | 73.6 | 73.7 | 73.7 | | 73.1 | 72.8 | 72.3 | 73.8 |
| 12 | S wild type | 56.9 | 56.7 | 55.7 | 57.9 | 55.9 | 100.0 | 99.5 | 91.6 | 89.7 |
| 13 | S2 | 52.5 | 53.2 | 52.3 | 54.4 | 52.0 | | 72.5 | 71.6 | 70.6 |
| 14 | S3 | 51.2 | 52.2 | 51.9 | 53.5 | 51.6 | | 70.9 | 69.9 | 69.1 |
| 15 | S6 | 53.4 | 53.8 | 53.2 | 54.7 | 53.3 | | 73.6 | 72.2 | 71.8 |
| 12 | S_ec wild type | 55.9 | 55.7 | 54.5 | 56.8 | 55.0 | | 99.5 | 91.6 | 90.2 |
| 13 | S2_ec | 51.7 | 52.3 | 51.4 | 53.6 | 51.2 | | 72.3 | 71.4 | 70.5 |
| 14 | S3_ec | 51.2 | 51.4 | 51.1 | 52.9 | 50.9 | | 70.9 | 69.9 | 69.1 |
| 15 | S6_ec | 52.6 | 53.1 | 52.4 | 53.8 | 52.4 | | 73.5 | 72.2 | 71.8 |
| 12 | SII wild type | 65.0 | 64.4 | 64.2 | 64.3 | 64.4 | | 99.5 | 93.7 | 90.1 |
| 13 | SII2 | 59.6 | 59.0 | 59.6 | 59.4 | 58.9 | | 73.1 | 73.5 | 71.9 |
| 14 | SII3 | 58.5 | 57.5 | 59.1 | 58.1 | 58.2 | | 71.5 | 71.5 | 70.2 |
| 15 | SII6 | 60.0 | 59.1 | 60.3 | 59.6 | 60.1 | | 74.9 | 74.7 | 74.2 |

Note:
— indicates no data.

According to an embodiment of the present disclosure, the above-described pharmaceutical formulation may further include at least one of the following additional technical features:

According to an embodiment of the present disclosure, the first nucleic acid fragment, the second nucleic acid fragment, and the third nucleic acid fragment are not linked. In some embodiments of the present disclosure, the mRNA prepared from the isolated nucleic acid fragment, when used as a vaccine, exhibits a good immune effect against FIPV, and can significantly improve various physiological indicators and the survival rate of the cat.

According to an embodiment of the present disclosure, a mass ratio of the first nucleic acid fragment to the second nucleic acid fragment is 10:1 to 1:10. Optionally, the mass ratio of the first nucleic acid fragment to the second nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to an embodiment of the present disclosure, a mass ratio of the second nucleic acid fragment to the third nucleic acid fragment is 10:1 to 1:10. Optionally, the mass ratio of the second nucleic acid fragment to the third nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to a preferred embodiment of the present disclosure, a mass ratio of the first nucleic acid fragment, the second nucleic acid fragment, and the third nucleic acid fragment is 1:1:1. In some embodiments of the present disclosure, when the three unlinked nucleic acid fragments are in the mass ratio of 1:1:1, the prepared RNA, when used as the vaccine, exhibits a good immune effect against FIPV and can significantly improve various physiological indicators and the survival rate of the cat.

According to a preferred embodiment of the present disclosure, the third nucleic acid fragment is selected from the SII protein. In some embodiments of the present disclosure, the SII protein selected as the third nucleic acid fragment to prepare an mRNA vaccine together with the first nucleic acid fragment and the second nucleic acid fragment has a good immune effect against FIPV and can significantly improve various physiological indicators and the survival rate of the cat.

According to an embodiment of the present disclosure, the first nucleic acid fragment, the second nucleic acid fragment, and the third nucleic acid fragment are linked.

According to an embodiment of the present disclosure, a mass ratio of the first nucleic acid fragment to the second nucleic acid fragment is 10:1 to 1:10. Optionally, the mass ratio of the first nucleic acid fragment to the second nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to an embodiment of the present disclosure, a mass ratio of the second nucleic acid fragment to the third nucleic acid fragment is 10:1 to 1:10. Optionally, the mass ratio of the second nucleic acid fragment to the third nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to a preferred embodiment of the present disclosure, a mass ratio of the first nucleic acid fragment, the second nucleic acid fragment, and the third nucleic acid fragment is 1:1:1.

According to an embodiment of the present disclosure, the first nucleic acid fragment is linked to the second nucleic acid fragment, and the third nucleic acid fragment is not linked to each of the first nucleic acid fragment and the second nucleic acid fragment; or the first nucleic acid fragment is linked to the third nucleic acid fragment, and the second nucleic acid fragment is not linked to each of the first nucleic acid fragment and the third nucleic acid fragment; or the second nucleic acid fragment is linked to the third nucleic acid fragment, and the first nucleic acid fragment is not linked to each of the second nucleic acid fragment and the third nucleic acid fragment.

According to an embodiment of the present disclosure, the 3′ end of the first nucleic acid fragment is linked to the 5′ end of the second nucleic acid fragment, and the third nucleic acid fragment is not linked to each of the first nucleic acid fragment and the second nucleic acid fragment; or the 3′ end of the second nucleic acid fragment is linked to the 5′ end of the first nucleic acid fragment, and the third nucleic acid fragment is not linked to each of the first nucleic acid fragment and the second nucleic acid fragment; or the 3′ end of the first nucleic acid fragment is linked to the 5′ end of the third nucleic acid fragment, and the second nucleic acid fragment is not linked to each of the first nucleic acid fragment and the third nucleic acid fragment; or the 3′ end of the third nucleic acid fragment is linked to the 5′ end of the first nucleic acid fragment, and the second nucleic acid fragment is not linked to each of the first nucleic acid fragment and the third nucleic acid fragment; or the 3′ end of the second nucleic acid fragment is linked to the 5′ end of the third nucleic acid fragment, and the first nucleic acid fragment is not linked to each of the second nucleic acid fragment and the third nucleic acid fragment; or the 3′ end of the third nucleic acid fragment is linked to the 5′ end of the second nucleic acid fragment, and the first nucleic acid fragment is not linked to each of the second nucleic acid fragment and the third nucleic acid fragment.

According to an embodiment of the present disclosure, the 3′ end of the first nucleic acid fragment is linked to the 5′ end of the second nucleic acid fragment, and the 3′ end of the second nucleic acid fragment is linked to the 5′ end of the third nucleic acid fragment; or the 3′ end of the first nucleic acid fragment is linked to the 5′ end of the third nucleic acid fragment, and the 3′ end of the third nucleic acid fragment is linked to the 5′ end of the second nucleic acid fragment; or the 3′ end of the second nucleic acid fragment is linked to the 5′ end of the first nucleic acid fragment, and the 3′ end of the first nucleic acid fragment is linked to the 5′ end of the third nucleic acid fragment; or the 3′ end of the second nucleic acid fragment is linked to the 5′ end of the third nucleic acid fragment, and the 3′ end of the third nucleic acid fragment is linked to the 5′ end of the first nucleic acid fragment; or the 3′ end of the third nucleic acid fragment is linked to the 5′ end of the first nucleic acid fragment, and the 3′ end of the first nucleic acid fragment is linked to the 5′ end of the second nucleic acid fragment; or the 3′ end of the third nucleic acid fragment is linked to the 5′ end of the second nucleic acid fragment, and the 3′ end of the second nucleic acid fragment is linked to the 5′ end of the first nucleic acid fragment.

In an exemplary embodiment of the present disclosure, the connection modes of the M, N, S, S_ec, or SII protein of feline infectious peritonitis virus (FIPV) can be combined arbitrarily.

According to an embodiment of the present disclosure, the M protein has the amino acid sequence as set forth in SEQ ID NO: 1.

According to an embodiment of the present disclosure, the M protein has the amino acid sequence with at least 89% homology to the amino acid sequence as set forth in SEQ ID NO: 1, and an amino acid at site 90 is Y, an amino acid at site 102 is V, an amino acid at site 120 is I, an amino acid at site 144 is A, and an amino acid at site 180 is L.

According to an embodiment of the present disclosure, the M protein has the amino acid sequence as set forth in SEQ ID NO: 1.

According to an embodiment of the present disclosure, the N protein has an amino acid sequence with at least 91% homology to the amino acid sequence as set forth in SEQ ID NO: 2.

According to an embodiment of the present disclosure, the N protein has the amino acid sequence as set forth in SEQ ID NO: 2.

According to an embodiment of the present disclosure, the S protein has an amino acid sequence with at least 45% homology to the amino acid sequence as set forth in SEQ ID NO: 3.

According to an embodiment of the present disclosure, the S protein has an amino acid sequence with at least 45% homology to the amino acid sequence as set forth in SEQ ID NO: 3, and an amino acid at site 515 is V, an amino acid at site 577 is Q, an amino acid at site 1385 is V, an amino acid at site 1386 is V, an amino acid at site 1397 is F, and an amino acid at site 1415 is I.

According to an embodiment of the present disclosure, the S protein has the amino acid sequence as set forth in SEQ ID NO: 3.

According to an embodiment of the present disclosure, the Sec protein has an amino acid sequence with at least 43% homology to amino acids at sites 1 to 1374 of an amino acid sequence as set forth in SEQ ID NO: 3.

According to an embodiment of the present disclosure, the Sec protein has the amino acid sequence with at least 43% homology to the amino acids at sites 1 to 1374 of the amino acid sequence as set forth in SEQ ID NO: 3, and an amino acid at site 515 is V and an amino acid at site 577 is Q.

According to an embodiment of the present disclosure, the Sec protein has the amino acid sequence of sites 1 to 1374 of SEQ ID NO: 3.

According to an embodiment of the present disclosure, the SII protein has an amino acid sequence with at least 62% homology to amino acids at sites 782 to 1433 of the amino acid sequence as set forth in SEQ ID NO: 3.

According to an embodiment of the present disclosure, the SII protein has the amino acid sequence with at least 62% homology to amino acids at sites 782 to 1433 of the amino acid sequence as set forth in SEQ ID NO: 3, and an amino acid at site 1385 is V, an amino acid at site 1386 is V, an amino acid at site 1397 is F, and an amino acid at site 1415 is I.

According to an embodiment of the present disclosure, the SII protein has the amino acid sequence of sites 782 to 1433 of SEQ ID NO: 3.

According to an embodiment of the present disclosure, the first nucleic acid fragment has a nucleotide sequence with at least 67% homology to a sequence as set forth in any one of SEQ ID NO: 4 to SEQ ID NO: 7.

According to an embodiment of the present disclosure, the first nucleic acid fragment has a nucleotide sequence as set forth in any one of SEQ ID NO: 4 to SEQ ID NO: 7.

According to an embodiment of the present disclosure, the second nucleic acid fragment has a nucleotide sequence with at least 70% homology to a sequence as set forth in any one of SEQ ID NO: 8 to SEQ ID NO: 11.

According to an embodiment of the present disclosure, the second nucleic acid fragment has a nucleotide sequence as set forth in any one of SEQ ID NO: 8 to SEQ ID NO: 11.

According to an embodiment of the present disclosure, the third nucleic acid fragment has a nucleotide sequence with at least 51% homology to nucleotides at sites 1 to 4122 of a sequence as set forth in any one of SEQ ID NO: 12 to SEQ ID NO: 15.

According to an embodiment of the present disclosure, the third nucleic acid fragment has a nucleotide sequence of sites 1 to 4122 of a sequence as set forth in any one of SEQ ID NO: 12 to SEQ ID NO: 15.

According to an embodiment of the present disclosure, the third nucleic acid fragment has a nucleotide sequence with at least 57% homology to nucleotides at sites 2344 to 4299 of a sequence as set forth in any one of SEQ ID NO: 12 to SEQ ID NO: 15.

According to an embodiment of the present disclosure, the third nucleic acid fragment has a nucleotide sequence of sites 2344 to 4299 of any one of SEQ ID NO: 12 to SEQ ID NO: 15.

According to an embodiment of the present disclosure, the third nucleic acid fragment has a nucleotide sequence with at least 51% homology to a sequence as set forth in any one of SEQ ID NO: 12 to SEQ ID NO: 15.

According to an embodiment of the present disclosure, the third nucleic acid fragment has a nucleotide sequence as set forth in any one of SEQ ID NO: 12 to SEQ ID NO: 15.

According to an embodiment of the present disclosure, the pharmaceutical formulation further includes a fourth nucleic acid fragment. The fourth nucleic acid fragment encodes a signal peptide sequence of MHC I (major histocompatibility complex I) or a sequence having a similar function to a signal peptide of MHC I. According to an embodiment of the present disclosure, the addition of the signal peptide of MHC I to the N-terminus of an antigen sequence can enable a ribosome to attach to an endoplasmic reticulum membrane and guide the intracellular transport of the protein.

It should be noted that, in the present disclosure, the sequence having the similar function to the signal peptide of MHC I can also enable the ribosome to attach to the endoplasmic reticulum membrane and guide the intracellular transport of the protein.

According to an embodiment of the present disclosure, the signal peptide sequence of MHC I includes no transmembrane region.

According to an embodiment of the present disclosure, the signal peptide sequence of MHC I has the amino acid sequence as set forth in SEQ ID NO: 16.

According to an embodiment of the present disclosure, the fourth nucleic acid fragment has a nucleotide sequence as set forth in any one of SEQ ID NO: 17 to SEQ ID NO: 22.

According to an embodiment of the present disclosure, the fifth nucleic acid fragment is located at the 5′ end of the nucleic acid fragment or the 5′ end of the nucleic acid molecule.

It should be noted that, the fourth nucleic acid fragment is located upstream of the nucleic acid fragment or the nucleic acid molecule, optionally, at the 5′ end of the nucleic acid fragment or the 5′ end of the nucleic acid molecule.

According to an embodiment of the present disclosure, the pharmaceutical formulation further includes a fifth nucleic acid fragment. The fifth nucleic acid fragment encodes an MITD (a molecule transport signal of MHC I or a molecule transport signal of major histocompatibility complex I) sequence or a sequence having a similar function to MITD. According to an embodiment of the present disclosure, the addition of the MITD sequence to the C-terminus of the nucleic acid molecule can stimulate the proliferation of CD4⁺ T cells and induce the production of more cytokines.

It should be noted that, in the present disclosure, the sequence having the similar function to MITD can also stimulate the proliferation of the CD4⁺ T cells, induce the production of cytokines, and trigger the immune response in animals.

According to an embodiment of the present disclosure, the MITD sequence has the amino acid sequence as set forth in SEQ ID NO: 23.

According to an embodiment of the present disclosure, the fifth nucleic acid fragment has a nucleotide sequence as set forth in any one of SEQ ID NO: 24 to SEQ ID NO: 32.

According to an embodiment of the present disclosure, the fifth nucleic acid fragment is located at the 3′ end of the nucleic acid fragment or the 3′ end of the nucleic acid molecule.

It should be noted that, the fifth nucleic acid fragment is located downstream of the nucleic acid fragment or the nucleic acid molecule, optionally at the 3′ end of the nucleic acid fragment or the 3′ end of the nucleic acid molecule.

According to an embodiment of the present disclosure, the nucleic acid fragment or nucleic acid molecule is a linear molecule.

According to an embodiment of the present disclosure, the nucleic acid fragment or nucleic acid molecule has a nucleotide sequence shown in Table 4.

According to an embodiment of the present disclosure, the pharmaceutical formulation further includes a pharmaceutical carrier. The pharmaceutical carrier includes at least one of a liposome, an exosome, a polymer carrier, a viral vector, or a nanoparticle.

It should be noted that, the pharmaceutical carrier refers to a carrier that does not cause obvious irritation to the subject and does not eliminate the biological activity and properties of the pharmaceutical formulation.

In a second aspect of the present disclosure, the present disclosure provides a method for preparing the pharmaceutical formulation described in the first aspect. According to an embodiment of the present disclosure, the method includes: mixing a first nucleic acid fragment, a second nucleic acid fragment, and a third nucleic acid fragment in a predetermined ratio to obtain the pharmaceutical formulation.

The first nucleic acid fragment encodes an M protein of feline infectious peritonitis virus; the second nucleic acid fragment encodes an N protein of feline infectious peritonitis virus; the third nucleic acid fragment encodes an S protein, an Sec protein, or an SII protein of feline infectious peritonitis virus; and the nucleic acid fragment is mRNA.

According to an embodiment of the present disclosure, the method can be used to prepare a pharmaceutical formulation for preventing or treating a related disease caused by feline infectious peritonitis virus.

According to an embodiment of the present disclosure, a mass ratio of the first nucleic acid fragment to the second nucleic acid fragment is 10:1 to 1:10. Optionally, the mass ratio of the first nucleic acid fragment to the second nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to an embodiment of the present disclosure, a mass ratio of the second nucleic acid fragment to the third nucleic acid fragment is 10:1 to 1:10. Optionally, the mass ratio of the second nucleic acid fragment to the third nucleic acid fragment is 1:1, or 1:2, or 1:3, or 1:4, or 1:5, or 1:6, or 1:7, or 1:8, or 1:9, or 1:10, or 10:1, or 9:1, or 8:1, or 7:1, or 6:1, or 5:1, or 4:1, or 3:1, or 2:1.

According to an embodiment of the present disclosure, a mass ratio of the first nucleic acid fragment, the second nucleic acid fragment, and the third nucleic acid fragment is 1:1:1.

In a third aspect of the present disclosure, the present disclosure provides an isolated nucleic acid molecule. According to an embodiment of the present disclosure, the nucleic acid molecule includes: a first nucleic acid fragment encoding an M protein of feline infectious peritonitis virus; a second nucleic acid fragment encoding an N protein of feline infectious peritonitis virus; and a third nucleic acid fragment encoding an S protein, S_ec protein, or SII protein of feline infectious peritonitis virus. The first nucleic acid fragment, the second nucleic acid fragment, and the third nucleic acid fragment are linked. The nucleic acid molecule is mRNA. According to an embodiment of the present disclosure, the nucleic acid molecule expressing the M protein, the N protein, the S protein, the S_ec protein, or the SII protein of feline infectious peritonitis virus can stimulate the cell-mediated immune response in animals.

It should be noted that, in the present disclosure, the M protein, the N protein, the S protein, the S_ec protein, or the SII protein of the wild-type FIPV can also be adaptively modified as desired to reduce the virulence of FIPV, while not affecting its three-dimensional structure and retaining its immunogenicity, so as to prepare a new type of FIPV vaccine. According to the sequence alignment results (Table 1 and Table 2), the M protein has an amino acid sequence with at least 89% homology to SEQ ID NO: 1, and the nucleic acid fragment encoding the M protein has a nucleotide sequence with at least 67% homology to a sequence as set forth in any one of SEQ ID NO: 4 to SEQ ID NO: 7; and the N protein has an amino acid sequence with at least 91% homology to SEQ ID NO: 2, and the nucleic acid fragment encoding the N protein has a nucleotide sequence with at least 70% homology to a sequence as set forth in any one of SEQ ID NO: 8 to SEQ ID NO: 11. The S protein has an amino acid sequence with at least 45% homology to SEQ ID NO: 3, and the nucleic acid fragment encoding the S protein has a nucleotide sequence with at least 51% homology to a sequence as set forth in any one of SEQ ID NO: 12 to SEQ ID NO: 15; the Sec protein has an amino acid sequence with at least 43% homology to amino acids at sites 1 to 1374 of SEQ ID NO: 3, and the nucleic acid fragment encoding the S_ec protein has a nucleotide sequence with at least 51% homology to nucleotides at sites 1 to 4122 of a sequence as set forth in any one of SEQ ID NO: 12 to SEQ ID NO: 15; and the SII protein has an amino acid sequence with at least 62% homology to amino acids at sites 782 to 1433 of SEQ ID NO: 3, and the nucleic acid fragment encoding the SII protein has a nucleotide sequence with at least 57% homology to nucleotides at sites 2344 to 4299 of a sequence as set forth in any of SEQ ID NO: 12 to SEQ ID NO: 15. The FIPV vaccine is not particularly limited, as long as it can produce the receptor binding domains of the modified M protein, N protein, S protein, Sec protein, or SII protein of FIPV in the living organism, has immunogenicity and can stimulate the living organism to produce a corresponding immune response. In addition, the pharmaceutical formulation can be used to stimulate the immune response of all animals that can be infected with feline infectious peritonitis virus, including but not limited to a cat.

According to an embodiment of the present disclosure, the above-described nucleic acid molecule may further include at least one of the following additional technical features:

According to an embodiment of the present disclosure, the first nucleic acid fragment is linked to the second nucleic acid fragment, and the third nucleic acid fragment is not linked to each of the first nucleic acid fragment and the second nucleic acid fragment; or the first nucleic acid fragment is linked to the third nucleic acid fragment, and the second nucleic acid fragment is not linked to each of the first nucleic acid fragment and the third nucleic acid fragment; or the second nucleic acid fragment is linked to the third nucleic acid fragment, and the first nucleic acid fragment is not linked to each of the second nucleic acid fragment and the third nucleic acid fragment.

According to an embodiment of the present disclosure, the 3′ end of the first nucleic acid fragment is linked to the 5′ end of the second nucleic acid fragment, and the third nucleic acid fragment is not linked to each of the first nucleic acid fragment and the second nucleic acid fragment; or the 3′ end of the second nucleic acid fragment is linked to the 5′ end of the first nucleic acid fragment, and the third nucleic acid fragment is not linked to each of the first nucleic acid fragment and the second nucleic acid fragment; or the 3′ end of the first nucleic acid fragment is linked to the 5′ end of the third nucleic acid fragment, and the second nucleic acid fragment is not linked to each of the first nucleic acid fragment and the third nucleic acid fragment; or the 3′ end of the third nucleic acid fragment is linked to the 5′ end of the first nucleic acid fragment, and the second nucleic acid fragment is not linked to each of the first nucleic acid fragment and the third nucleic acid fragment; or the 3′ end of the second nucleic acid fragment is linked to the 5′ end of the third nucleic acid fragment, and the first nucleic acid fragment is not linked to each of the second nucleic acid fragment and the third nucleic acid fragment; or the 3′ end of the third nucleic acid fragment is linked to the 5′ end of the second nucleic acid fragment, and the first nucleic acid fragment is not linked to each of the second nucleic acid fragment and the third nucleic acid fragment.

According to an embodiment of the present disclosure, the 3′ end of the first nucleic acid fragment is linked to the 5′ end of the second nucleic acid fragment, and the 3′ end of the second nucleic acid fragment is linked to the 5′ end of the third nucleic acid fragment; or the 3′ end of the first nucleic acid fragment is linked to the 5′ end of the third nucleic acid fragment, and the 3′ end of the third nucleic acid fragment is linked to the 5′ end of the second nucleic acid fragment; or the 3′ end of the second nucleic acid fragment is linked to the 5′ end of the first nucleic acid fragment, and the 3′ end of the first nucleic acid fragment is linked to the 5′ end of the third nucleic acid fragment; or the 3′ end of the second nucleic acid fragment is linked to the 5′ end of the third nucleic acid fragment, and the 3′ end of the third nucleic acid fragment is linked to the 5′ end of the first nucleic acid fragment; or the 3′ end of the third nucleic acid fragment is linked to the 5′ end of the first nucleic acid fragment, and the 3′ end of the first nucleic acid fragment is linked to the 5′ end of the second nucleic acid fragment; or the 3′ end of the third nucleic acid fragment is linked to the 5′ end of the second nucleic acid fragment, and the 3′ end of the second nucleic acid fragment is linked to the 5′ end of the first nucleic acid fragment.

In an exemplary embodiment of the present disclosure, the connection modes of the M, N, S, S_ec, or SII protein of feline infectious peritonitis virus (FIPV) can be combined arbitrarily.

According to an embodiment of the present disclosure, the M protein has the amino acid sequence as set forth in SEQ ID NO: 1.

According to an embodiment of the present disclosure, the M protein has the amino acid sequence with at least 89% homology to the amino acid sequence as set forth in SEQ ID NO: 1, and an amino acid at site 90 is Y, an amino acid at site 102 is V, an amino acid at site 120 is I, an amino acid at site 144 is A, and an amino acid at site 180 is L.

According to an embodiment of the present disclosure, the M protein has an amino acid sequence as set forth in SEQ ID NO: 1.

According to an embodiment of the present disclosure, the N protein has an amino acid sequence with at least 91% homology to an amino acid sequence as set forth in SEQ ID NO: 2.

According to an embodiment of the present disclosure, the N protein has the amino acid sequence as set forth in SEQ ID NO: 2.

According to an embodiment of the present disclosure, the S protein has an amino acid sequence with at least 45% homology to the amino acid sequence as set forth in SEQ ID NO: 3.

According to an embodiment of the present disclosure, the S protein has the amino acid sequence with at least 45% homology to the amino acid sequence as set forth in SEQ ID NO: 3, and an amino acid at site 515 is V, an amino acid at site 577 is Q, an amino acid at site 1385 is V, an amino acid at site 1386 is V, an amino acid at site 1397 is F, and an amino acid at site 1415 is I.

According to an embodiment of the present disclosure, the S protein has the amino acid sequence as set forth in SEQ ID NO: 3.

According to an embodiment of the present disclosure, the Sec protein has an amino acid sequence with at least 43% homology to amino acids at sites 1 to 1374 of an amino acid sequence as set forth in SEQ ID NO: 3.

According to an embodiment of the present disclosure, the Sec protein has the amino acid sequence with at least 43% homology to the amino acids at sites 1 to 1374 of the amino acid sequence as set forth in SEQ ID NO: 3, and an amino acid at site 515 is V and an amino acid at site 577 is Q.

According to an embodiment of the present disclosure, the Sec protein has the amino acid sequence of sites 1 to 1374 of SEQ ID NO: 3.

According to an embodiment of the present disclosure, the SII protein has an amino acid sequence with at least 62% homology to amino acids at sites 782 to 1433 of an amino acid sequence as set forth in SEQ ID NO: 3.

According to an embodiment of the present disclosure, the SII protein has the amino acid sequence with at least 62% homology to amino acids at sites 782 to 1433 of the amino acid sequence as set forth in SEQ ID NO: 3, and an amino acid at site 1385 is V, an amino acid at site 1386 is V, an amino acid at site 1397 is F, and an amino acid at site 1415 is I.

According to an embodiment of the present disclosure, the SII protein has the amino acid sequence of sites 782 to 1433 of SEQ ID NO: 3.

According to an embodiment of the present disclosure, the first nucleic acid fragment has a nucleotide sequence with at least 67% homology to a sequence as set forth in any one of SEQ ID NO: 4 to SEQ ID NO: 7.

According to an embodiment of the present disclosure, the second nucleic acid fragment has a nucleotide sequence with at least 70% homology to a sequence as set forth in any one of SEQ ID NO: 8 to SEQ ID NO: 11.

According to an embodiment of the present disclosure, the second nucleic acid fragment has a nucleotide sequence as set forth in any one of SEQ ID NO: 8 to SEQ ID NO: 11.

According to an embodiment of the present disclosure, the third nucleic acid fragment has a nucleotide sequence with at least 51% homology to nucleotides at sites 1 to 4122 of a sequence as set forth in any one of SEQ ID NO: 12 to SEQ ID NO: 15.

According to an embodiment of the present disclosure, the third nucleic acid fragment has a nucleotide sequence of sites 1 to 4122 of a sequence as set forth in any one of SEQ ID NO: 12 to SEQ ID NO: 15.

According to an embodiment of the present disclosure, the third nucleic acid fragment has a nucleotide sequence with at least 57% homology to nucleotides at sites 2344 to 4299 of a sequence as set forth in any one of SEQ ID NO: 12 to SEQ ID NO: 15.

According to an embodiment of the present disclosure, the third nucleic acid fragment has a nucleotide sequence of sites 2344 to 4299 of any one of SEQ ID NO: 12 to SEQ ID NO: 15.

According to an embodiment of the present disclosure, the third nucleic acid fragment has a nucleotide sequence with at least 51% homology to a sequence as set forth in any one of SEQ ID NO: 12 to SEQ ID NO: 15.

According to an embodiment of the present disclosure, the third nucleic acid fragment has a nucleotide sequence as set forth in any one of SEQ ID NO: 12 to SEQ ID NO: 15.

According to an embodiment of the present disclosure, the nucleic acid molecule further includes a fourth nucleic acid fragment. The fourth nucleic acid fragment encodes a signal peptide sequence of MHC I (major histocompatibility complex I) or a sequence having a similar function to a signal peptide of MHC I. According to an embodiment of the present disclosure, the addition of the signal peptide of MHC I to the N-terminus of an antigen sequence can enable a ribosome to attach to an endoplasmic reticulum membrane, thereby guiding the intracellular transport of the protein.

It should be noted that, in the present disclosure, the sequence having the similar function to the signal peptide of MHC I can also enable the ribosome to attach to the endoplasmic reticulum membrane, thereby guiding the intracellular transport of the protein.

According to an embodiment of the present disclosure, the signal peptide sequence of MHC I includes no transmembrane region.

According to an embodiment of the present disclosure, the signal peptide sequence of MHC I has the amino acid sequence as set forth in SEQ ID NO: 16.

According to an embodiment of the present disclosure, the fourth nucleic acid fragment has a nucleotide sequence as set forth in any one of SEQ ID NO: 17 to SEQ ID NO: 22.

According to an embodiment of the present disclosure, the fourth nucleic acid fragment is located at the 5′ end of the nucleic acid fragment or the 5′ end of the nucleic acid molecule.

It should be noted that, the fourth nucleic acid fragment is located upstream of the nucleic acid fragment or the nucleic acid molecule, optionally, at the 5′ end of the nucleic acid fragment or the 5′ end of the nucleic acid molecule.

According to an embodiment of the present disclosure, the nucleic acid molecule further includes a fifth nucleic acid fragment. The fifth nucleic acid fragment encodes an MITD (a molecule transport signal of MHC I or a molecule transport signal of major histocompatibility complex I) sequence or a sequence having a similar function to MITD. According to an embodiment of the present disclosure, the addition of the MITD sequence to the C-terminus of the nucleic acid molecule can stimulate the proliferation of CD4⁺ T cells and induce the production of more cytokines.

It should be noted that, in the present disclosure, the sequence having the similar function to MITD can also stimulate the proliferation of the CD4⁺ T cells, induce the production of cytokines, and trigger the immune response in animals.

According to an embodiment of the present disclosure, the MITD sequence has the amino acid sequence as set forth in SEQ ID NO: 23.

According to an embodiment of the present disclosure, the fifth nucleic acid fragment has a nucleotide sequence as set forth in any one of SEQ ID NO: 24 to SEQ ID NO: 32.

According to an embodiment of the present disclosure, the fifth nucleic acid fragment is located at the 3′ end of the nucleic acid fragment or the 3′ end of the nucleic acid molecule.

It should be noted that, the fifth nucleic acid fragment is located downstream of the nucleic acid fragment or the nucleic acid molecule, optionally at the 3′ end of the nucleic acid fragment or the 3′ end of the nucleic acid molecule.

According to an embodiment of the present disclosure, the nucleic acid fragment or nucleic acid molecule is a linear molecule.

According to an embodiment of the present disclosure, the nucleic acid fragment or nucleic acid molecule has a nucleotide sequence shown in Table 4.

In a fourth aspect of the present disclosure, the present disclosure provides an expression vector. According to an embodiment of the present disclosure, the expression vector carries the nucleic acid molecule in the third aspect of the present disclosure. According to an embodiment of the present disclosure, the expression vector can be expressed in cells, bacteria, yeasts, or felines.

According to an embodiment of the present disclosure, the above-described expression vector may further include at least one of the following additional technical features.

According to an embodiment of the present disclosure, the expression vector is a non-viral vector.

In a fifth aspect of the present disclosure, the present disclosure provides a recombinant virus. According to an embodiment of the present disclosure, the recombinant virus carries the nucleic acid molecule in the third aspect of the present disclosure. The recombinant virus including the nucleic acid molecule in the third aspect can be propagated in large quantities, and the recombinant virus plays an important role in vaccine research and development.

In a sixth aspect of the present disclosure, the present disclosure provides a liposome. According to an embodiment of the present disclosure, the liposome includes a liposome carrier and a nucleic acid fragment. The nucleic acid fragment is as defined in the first aspect of the present disclosure and the third aspect of the present disclosure. The liposome including the liposome carrier and the nucleic acid fragment plays an important role in improving nucleic acid stability, increasing cellular uptake rate, reducing toxic and side effects, improving delivery efficiency, and other aspects.

In a seventh aspect of the present disclosure, the present disclosure provides a vaccine. According to an embodiment of the present disclosure, the vaccine includes the pharmaceutical formulation in the first aspect of the present disclosure, the nucleic acid molecule in the third aspect of the present disclosure, the expression vector in the fourth aspect of the present disclosure, the recombinant virus in the fifth aspect of the present disclosure, or the liposome in the sixth aspect of the present disclosure. According to an embodiment of the present disclosure, the above-described vaccine can efficiently activate the cell-mediated immune response in animals. In addition, the vaccine contains only proteins capable of activating the cellular immune response, which avoids the occurrence of toxic and side effects and has higher safety.

According to an embodiment of the present disclosure, the above-described vaccine may further include at least one of the following additional technical features.

According to an embodiment of the present disclosure, the vaccine further includes an adjuvant.

According to an embodiment of the present disclosure, the adjuvant includes at least one of a TLR agonist or Mn²⁺.

According to an embodiment of the present disclosure, the TLR agonist includes at least one of CpG, R837, MPLA, or derivatives thereof.

In an eighth aspect of the present disclosure, the present disclosure provides a recombinant cell. According to an embodiment of the present disclosure, the recombinant cell carries a nucleic acid fragment, the nucleic acid molecule in the third aspect of the present disclosure, the expression vector in the fourth aspect of the present disclosure, or the recombinant virus in the fifth aspect of the present disclosure.

The nucleic acid fragment includes at least one of a first nucleic acid fragment, a second nucleic acid fragment, or a third nucleic acid fragment. The first nucleic acid fragment encodes an M protein of feline infectious peritonitis virus; the second nucleic acid fragment encodes an N protein of feline infectious peritonitis virus; the third nucleic acid fragment encodes an S protein, Sec protein, or SII protein of feline infectious peritonitis virus; and the first nucleic acid fragment, the second nucleic acid fragment, and the third nucleic acid fragment are linked or not linked.

According to an embodiment of the present disclosure, the recombinant cell is used to package a virus carrying the nucleic acid molecule for preparing a nucleic acid vaccine or expressing the M, N, S, S_ec, or SII protein of FIPV, so as to stimulate a strong immune response in the animals.

In a ninth aspect of the present disclosure, the present disclosure provides a method for constructing a feline infectious peritonitis virus vaccine. According to an embodiment of the present disclosure, the method includes: introducing a nucleic acid fragment, the nucleic acid molecule in the third aspect of the present disclosure, the expression vector in the fourth aspect of the present disclosure, or the recombinant virus in the fifth aspect of the present disclosure into a recipient cell.

The nucleic acid fragment includes at least one of a first nucleic acid fragment, a second nucleic acid fragment, or a third nucleic acid fragment. The first nucleic acid fragment encodes an M protein of feline infectious peritonitis virus; the second nucleic acid fragment encodes an N protein of feline infectious peritonitis virus; the third nucleic acid fragment encodes an S protein, Sec protein, or SII protein of feline infectious peritonitis virus; and the first nucleic acid fragment, the second nucleic acid fragment, and the third nucleic acid fragment are linked or not linked.

The method according to an embodiment of the present disclosure can package the virus carrying the nucleic acid molecule for preparing the nucleic acid vaccine. The method for constructing the feline infectious peritonitis virus vaccine is safe, simple, and efficient.

According to an embodiment of the present disclosure, the above-described method for constructing the feline infectious peritonitis virus vaccine may further include at least one of the following additional technical features:

According to an embodiment of the present disclosure, the method further includes, prior to said introducing into the recipient cell, encapsulating the nucleic acid, the expression vector, or the recombinant virus with an encapsulation carrier.

According to an embodiment of the present disclosure, the encapsulation carrier is selected from at least one of a liposome, exosome, polymer carrier, viral vector, or nanoparticle.

According to an embodiment of the present disclosure, the encapsulation carrier is a nanoparticle. The selection of the nanoparticle for encapsulating mRNA can protect mRNA from degradation, and facilitate the delivery of mRNA into the cell by binding to the cell membrane.

According to a specific embodiment of the present disclosure, the encapsulation carrier is a lipid nanoparticle.

According to an embodiment of the present disclosure, the recipient cell is a CRFK cell, HEK293FT cell, HEK293T cell, BHK cell, or insect cell.

According to an embodiment of the present disclosure, the recipient cell is a CRFK cell. According to an embodiment of the present disclosure, the CFRK cell does not cause an immune rejection response in the subject animal.

In a tenth aspect of the present disclosure, the present disclosure provides use of the pharmaceutical formulation in the first aspect of the present disclosure, the nucleic acid molecule in the third aspect of the present disclosure, the expression vector in the fourth aspect of the present disclosure, the recombinant virus in the fifth aspect of the present disclosure, or the liposome in the sixth aspect of the present disclosure in the manufacture of a medicament or vaccine. According to an embodiment of the present disclosure, the medicament or vaccine is used for preventing or treating a disease related to feline infectious peritonitis virus infection. According to an embodiment of the present disclosure, the medicament or vaccine prepared based on the above-described nucleic acid molecule, expression vector, recombinant virus, or recombinant cell exhibits high safety and can activate the cell-mediated immune response in animals in a short period of time.

In an eleventh aspect of the present disclosure, the present disclosure provides a method for preventing or treating a disease caused by feline infectious peritonitis virus infection. According to an embodiment of the present disclosure, the method includes administering to a subject the pharmaceutical formulation in the first aspect of the present disclosure, the nucleic acid molecule in the third aspect of the present disclosure, the expression vector in the fourth aspect of the present disclosure, the recombinant virus in the fifth aspect of the present disclosure, the liposome in the sixth aspect of the present disclosure, the vaccine in the seventh aspect of the present disclosure, or the recombinant cell in the eighth aspect of the present disclosure. According to an embodiment of the present disclosure, administering an effective dose of the pharmaceutical formulation, nucleic acid molecule, expression vector, recombinant virus, liposome, vaccine, or recombinant cell to the subject infected with FIPV can significantly improve various physiological indicators and a survival rate of the subject. In addition, the above-described treatment method exhibits a good immune effect against all prevalent strains of FIPV

As used herein, the term “effective dose” refers to an amount that produces a function or activity in subjects and is acceptable to subjects.

The effective amount of the pharmaceutical formulation, nucleic acid molecule, expression vector, recombinant virus, liposome, vaccine, or recombinant cell of the present disclosure can vary with the administration mode, the severity of FIPV infection in the subject, etc. The selection of the preferred effective amount can be determined by a person of ordinary skill in the art based on various factors (e.g., through clinical trials). Such factors include, but are not limited to: pharmacokinetic parameters of the active component such as bioavailability, metabolism, half-life period, etc.; and the severity of the FIPV infection in the subject, a weight of the subject, an immune status of the subject, a route of administration, etc. For example, depending on exigencies of the therapeutic situation, several separate doses can be administered daily, or the dose can be proportionally reduced.

According to an embodiment of the present disclosure, the above-described method may further include at least one of the following technical features.

According to an embodiment of the present disclosure, the subject is selected from a cat.

In a twelfth aspect of the present disclosure, the present disclosure provides use of the pharmaceutical formulation in the first aspect, the nucleic acid molecule in the third aspect, the expression vector in the fourth aspect, the recombinant virus in the fifth aspect, the liposome in the sixth aspect, the vaccine in the seventh aspect, or the recombinant cell in the eighth aspect in the prevention or treatment of a disease caused by feline infectious peritonitis virus infection. According to an embodiment of the present disclosure, administering an effective dose of the pharmaceutical formulation, nucleic acid molecule, expression vector, recombinant virus, liposome, vaccine, or recombinant cell to a subject infected with FIPV can significantly improve various physiological indicators and a survival rate of the subject. In addition, the above-described therapeutic method exhibits a good immune effect against all prevalent strains of FIPV.

Additional aspects and advantages of the present disclosure will be provided at least in part in the following description, or will become apparent at least in part from the following description, or can be learned from practicing of the present disclosure.

### BRIEF DESCRIPTION OF THE DRAWINGS

The above and/or additional aspects and advantages of the present disclosure will become more apparent and readily understood from the following description of the embodiments taken in conjunction with the accompanying drawings.

FIG. 1 shows the detection results of the expression of the M protein, N protein, and S protein according to Example 1 of the present disclosure.

FIG. 2 shows the detection results of IFN-γ activation in PBMCs after immunization with the target mRNA encapsulated by LNP according to Example 2 of the present disclosure.

FIG. 3 shows the detection results of IFN-γ activation in PBMCs after immunization with the target mRNA encapsulated by LNP according to Example 3 of the present disclosure.

FIG. 4 shows the detection results of the expression of the target mRNA encapsulated by LNP according to Example 4 of the present disclosure.

FIG. 5 shows the changes in survival rate after viral challenge following immunization with the target mRNA encapsulated by LNP according to Example 4 of the present disclosure.

FIG. 6 shows the changes in survival rate after viral challenge following immunization with the target mRNA encapsulated by LNP according to Example 5 of the present disclosure.

FIG. 7 shows the detection results of the expression of the target mRNA encapsulated by LNP according to Example 6 of the present disclosure.

FIG. 8 shows the changes in survival rate after viral challenge following immunization with the target mRNA encapsulated by LNP according to Example 6 of the present disclosure.

FIG. 9 shows the changes in survival rate after viral challenge following immunization with the target mRNA encapsulated by LNP according to Example 7 of the present disclosure.

FIG. 10 shows the changes in survival rate after viral challenge following immunization with the target mRNA encapsulated by LNP according to Example 8 of the present disclosure.

### DETAILED DESCRIPTION

Embodiments of the present disclosure will be described in detail below, examples of which are illustrated in the accompanying drawings. The embodiments described below with reference to the drawings are illustrative only, and are intended to explain, and should not be construed as a limitation to the present disclosure.

In addition, terms “first” and “second” are only used for descriptive purposes, and cannot be understood as indicating or implying relative importance or implicitly indicating the number of indicated technical features. Therefore, the features defined with “first” and “second” may explicitly or implicitly include at least one of the features. In the description of the present disclosure, “a plurality of” means at least two, for example, two or three, unless specified otherwise.

It should be noted that, in the present disclosure, the terms “homology” and “identity” both refer to the similarity between amino acid or nucleotide sequences, and there is no specific distinction in their meanings.

### Beneficial Effects

The mRNA vaccine for preventing feline infectious peritonitis described in the present disclosure is prepared by constructing a vector encoding at least one of the M, N, S, S_ec, or SII protein of FIPV, and then preparing the mRNA vaccine for preventing FIPV through the lipid nanoparticle (LNP). The vaccine stimulates a subject to produce the cellular immune response. The vaccine, using mRNA containing the amino acid sequences of the M, N, S, S_ec, or SII of FIPV as its main component, has the advantages such as a simple preparation process, high safety, no toxic and side effects, and industrial producibility; administration of an extremely small dose of the vaccine can achieve a sufficiently protective effect, and it is superior to the existing therapeutic methods in terms of safety and effectiveness.

The sequences involved in the present disclosure are shown in Table 3.

TABLE 3

| SEQ ID NO: | Sequence | Description |
|---|---|---|
| 1 | ERYCAMQNTGSQCINGTDSSCSTCFERGGLIWHLANWNFSWS | M |
| | VILIVFITVLKYGRPQFSWLVYGIKMLIMWLLWPIVLALTIFNAY | |
| | SEYQVSRYVMFGFSVAGAVVTFALWMMYFVRSIQLYRRTKSW | |
| | WSFNPETNAILCVNALGRSYVLPLDGTPTGVTLTLLSGNLYAEG | |
| | FKMAGGLTIEHLPKYVMIATPSRTIVYTLVGKQLKATTATGWAY | |
| | YVKSKAGDYSTEARTDNLSEHEKLLHMV | |
| 2 | ATQGQRVNWGDEPSKRRGRSNSRGRKNNTIPLSFFNPIQLEPGS | N |
| | KFWSVCPRDFVPKGIGNKDQQIGYWNRQERYRIVKGQRKELPE | |
| | RWFFYFLGTGPQADAKFKDKIDGVFWVAKDGAMNKPTTLGTR | |
| | GTNNESKPLKFDGKIPPQFQLEVNRSRNNSRSGSQSRSVSRNRS | |
| | QSRGRQQSNNQNNVEDTIVAVLQKLGVTEKQRSRSKSRDRGDS | |
| | KPRDTTPNNANKHTWKKTAGKGDVTNFYGARSASANFGDSDL | |
| | VANGNAAKSYPQIAECVPSVSSMLFGSQWSAEDDGDQVKVTL | |
| | THTYYLPKDDAKTSQFLEQIDAYKRPSQVAKDQRQRKSRSKSA | |
| | EKKPEELSVTLVEAYTDVFDDTQVEMIDEVTN | |
| 3 | TTNNECIQVNVTQLAGNENLIRDFLFSNFKEEGSVVVGGYYPT | S |
| | EVWYNCSRTARTTAFQYFNNIHAFYFVMEAMENSTGNARGKP | |
| | LLFHVHGEPVSVIISAYRDDVQQRPLLKHGLVCITKNRHINYEQ | |
| | FTSNQWNSTCTGADRKIPFSVIPTDNGTKIYGLEWNDDFVTAYI | |
| | SGRSYHLNINTNWFNNVTLLYSRSSTATWEYSAAYAYQGVSNF | |
| | TYYKLNNTNGLKTYELCEDYEHCTGYATNVFAPTSGGYIPDGF | |
| | SFNNWFLLTNSSTFVSGRFVTNQPLLINCLWPVPSFGVAAQEFC | |
| | FEGAQFSQCNGVSLNNTVDVIRFNLNFTADVQSGMGATVFSLN | |
| | TTGGVILEISCYSDTVSESSSYSYGEIPFGITDGPRYCYVLYNGTA | |
| | LKYLGTLPPSVKEIAISKWGHFYINGYNFFSTFPIGCISFNLTTGV | |
| | SGAFWTIAYTSYTEALVQVENTAIKNVTYCNSHINNIKCSQLTA | |
| | NLNNGFYPVASSEVGFVNKSVVLLPSFFTYTAVNITIDLGMKLS | |
| | GYGQPIASTLSNITLPMQDNNTDVYCIRSNQFSVYVHSTCKSSL | |
| | WDNIFNQDCTDVLEATAVIKTGTCPFSFDKLNNYLTFNKFCLSL | |
| | SPVGANCKFDVAARTRTNEQVVRSLYVIYEEGDNIVGVPSDNS | |
| | GLHDLSVLHLDSCTDYNIYGRTGVGIIRRTNSTLLSGLYYTSLSG | |
| | DLLGFKNVSDGVIYSVTPCDVSAQAAVIDGAIVGAMTSINSELL | |
| | GLTHWTTTPNFYYYSIYNYTSERTRDTAIDSNDVDCEPVITYSNI | |
| | GVCKNGALVFINVTHSDGDVQPISTGNVTIPTNFTISVQVEYMQ | |
| | VYTTPVSIDCARYVCNGNPRCNKLLTQYVSACQTIEQALAMGA | |
| | RLENMEVDSMLFVSENALKLASVEAFNSTENLDSIYKEWPSIG | |
| | GSWLGGLKDILPSHNSKRKYGSAIEDLLFDKVVTSGLGTVDED | |
| | YKRCTGGYDIADLVCAQYYNGIMVLPGVANADKMTMYTASL | |
| | AGGITLGALGGGAVAIPFAVAVQARLNYVALQTDVLNKNQQILA | |
| | NAFNQAIGNITQAFGKVNDAIHQTSQGLATVAKALAKVQDVV | |
| | NTQGQALSHLTVQLQNNFQAISSSISDIYNRLDPPSADAQVDRLI | |
| | TGRLTALNAFVSQTLTRQAEVRASRQLAKDKVNECVRSQSQRF | |
| | GFCGNGTHLFSLANAAPNGMIFFHTVLLPTAYETVTAWSGICAS | |
| | DGDRTFGLVVKDVQLTLFRNLDDKFYLTPRTMYQPRVATSSDF | |
| | VQIEGCDVLFVNATVIDLPSIIPDYIDINQTVQDILENYRPNWTV | |
| | PEFTLDIFNATYLNLTGEIDDLEFRSEKLHNTTVELAILIDNINNT | |
| | LVNLEWLNRIETYVKWPWYVWLLIGLVVVFCIPLLLFCCFSTG | |
| | CCGCIGCLGSCCHSICSRRQFENYEPIEKVHVH | |
| 4 | GAACGCTACTGTGCCATGCAAAATACAGGCTCGCAGTGCATT | M wild type |
| | AATGGCACAGATTCATCATGTAGCACCTGTTTTGAACGTGGT | |
| | GGTCTTATTTGGCATCTGGCTAACTGGAACTTCAGCTGGTCTG | |
| | TAATATTGATTGTTTTTATAACAGTGTTAAAATATGGAAGACC | |
| | GCAATTCAGCTGGCTCGTTTATGGCATTAAAATGCTGATCATG | |
| | TGGCTATTATGGCCTATTGTTCTAGCGCTTACGATTTTTAATGC | |
| | ATACTCTGAGTACCAAGTTTCCAGATATGTAATGTTCGGCTTT | |
| | AGTGTTGCAGGTGCAGTTGTAACGTTTGCACTATGGATGATG | |
| | TATTTTGTGAGATCTATTCAGCTGTATAGACGGACCAAATCAT | |
| | GGTGGTCTTTTAATCCTGAAACCAATGCGATTCTTTGTGTCAA | |
| | TGCATTGGGTAGAAGCTATGTACTCCCTCTTGATGGCACTCCT | |
| | ACAGGTGTTACTCTTACCCTACTTTCAGGAAATCTATACGCTG | |
| | AAGGTTTTAAAATGGCTGGTGGTCTTACCATCGAGCATTTGC | |
| | CTAAATATGTCATGATTGCTACGCCTAGTAGAACCATCGTTTA | |
| | CACATTAGTTGGAAAACAACTAAAGGCAACTACTGCCACTG | |
| | GATGGGCTTACTATGTAAAATCTAAAGCTGGTGATTACTCAAC | |
| | AGAAGCACGTACTGATAATTTGAGTGAACATGAAAAATTATT | |
| | ACATATGGTG | |
| 5 | GAGAGATACTGTGCCATGCAGAACACAGGCAGCCAGTGCAT | M (M2) |
| | CAATGGAACAGACAGCAGCTGCAGCACCTGCTTTGAAAGAG | |
| | GAGGCCTGATCTGGCACCTGGCCAACTGGAACTTCAGCTGG | |
| | AGTGTGATCCTGATTGTCTTCATTACAGTGCTGAAGTACGGC | |
| | AGGCCCCAGTTCAGCTGGCTGGTGTACGGCATCAAGATGCTC | |
| | ATCATGTGGCTGCTGTGGCCCATTGTGTTGGCCCTCACCATCT | |
| | TCAATGCCTACAGCGAGTACCAGGTGTCCAGATACGTGATGT | |
| | TCGGCTTCTCAGTGGCAGGAGCCGTGGTGACCTTTGCCCTCT | |
| | GGATGATGTACTTTGTGAGGTCCATCCAGCTCTACAGAAGAA | |
| | CAAAGAGCTGGTGGAGCTTCAACCCAGAAACCAACGCCATC | |
| | CTGTGTGTCAATGCCCTGGGCAGATCCTATGTGCTGCCCCTG | |
| | GATGGCACCCCCACAGGCGTCACCCTCACCCTCCTGAGCGG | |
| | CAACCTGTACGCCGAGGGCTTCAAGATGGCCGGCGGCCTGA | |
| | CAATCGAGCACCTGCCCAAGTATGTGATGATTGCCACCCCCA | |
| | GCAGGACAATAGTCTACACCCTGGTGGGAAAACAGCTGAAG | |
| | GCTACCACAGCCACAGGCTGGGCCTACTACGTCAAGAGCAA | |
| | GGCCGGCGACTACAGCACAGAGGCCAGGACCGACAACCTGT | |
| | CCGAACATGAAAAACTCCTGCACATGGTC | |
| 6 | GAGAGGTACTGCGCCATGCAGAACACAGGCTCCCAGTGCAT | M (M3) |
| | CAACGGAACAGACTCCTCCTGCAGCACCTGCTTCGAGAGAG | |
| | GAGGCCTCATTTGGCACCTGGCCAACTGGAACTTCAGCTGGT | |
| | CAGTCATTCTGATAGTCTTCATAACAGTGCTGAAGTACGGCA | |
| | GGCCCCAGTTCTCCTGGCTCGTGTATGGCATCAAGATGCTGAT | |
| | CATGTGGCTGCTGTGGCCCATCGTCCTGGCCCTGACCATCTTC | |
| | AACGCCTACTCAGAGTACCAGGTCAGCAGGTACGTGATGTTC | |
| | GGCTTCTCCGTGGCCGGAGCAGTGGTGACCTTCGCCCTGTGG | |
| | ATGATGTACTTCGTGAGGAGCATCCAACTGTACAGGAGGACC | |
| | AAAAGCTGGTGGTCCTTCAACCCAGAAACCAACGCCATCCT | |
| | CTGCGTGAACGCCCTGGGCAGGTCCTACGTCCTCCCCCTGGA | |
| | CGGCACCCCCACCGGGGTCACCCTCACCCTCCTGTCAGGGA | |
| | ACCTGTACGCTGAGGGCTTCAAGATGGCTGGAGGCCTGACA | |
| | ATTGAACACCTGCCCAAGTACGTCATGATCGCAACACCCTCC | |
| | AGAACCATCGTCTACACCCTGGTGGGCAAGCAGCTGAAGGC | |
| | CACCACCGCCACCGGCTGGGCCTACTACGTCAAGTCCAAGG | |
| | CCGGCGACTACAGCACCGAGGCCAGGACCGACAACCTCTCA | |
| | GAGCACGAGAAGCTGCTGCACATGGTG | |
| 7 | GAGAGGTACTGTGCCATGCAGAACACCGGTTCCCAGTGCATC | M (M6) |
| | AACGGCACAGACTCCTCCTGCTCCACCTGCTTCGAGAGAGG | |
| | AGGCCTGATCTGGCACCTGGCAAACTGGAACTTCAGCTGGA | |
| | GCGTGATCCTGATAGTGTTCATAACCGTCCTGAAGTACGGCA | |
| | GACCACAGTTCTCATGGCTTGTCTATGGCATCAAGATGCTGAT | |
| | TATGTGGCTGCTTTGGCCTATCGTCCTGGCCCTGACCATCTTC | |
| | AACGCCTACTCTGAGTACCAGGTGTCAAGGTATGTCATGTTC | |
| | GGCTTCTCAGTGGCTGGAGCTGTGGTGACCTTTGCTCTGTGG | |
| | ATGATGTACTTCGTGAGGTCCATCCAGCTGTACAGGAGGACA | |
| | AAGTCATGGTGGTCCTTCAACCCAGAAACCAATGCCATCCTG | |
| | TGCGTCAACGCACTGGGCAGAAGCTACGTCCTACCACTGGA | |
| | CGGCACTCCTACAGGAGTGACCCTGACCCTGCTGTCAGGCA | |
| | ATCTGTACGCAGAGGGGTTCAAGATGGCCGGTGGCCTGACC | |
| | ATCGAGCATCTGCCTAAGTACGTGATGATCGCCACCCCTAGC | |
| | AGGACAATCGTGTACACCCTGGTGGGAAAGCAGCTAAAGGC | |
| | GACCACAGCCACAGGCTGGGCCTACTACGTGAAGTCCAAGG | |
| | CAGGGGACTATTCAACCGAGGCCAGGACCGACAACCTGTCA | |
| | GAGCACGAGAAGCTGCTGCACATGGTC | |
| 8 | GCCACACAGGGACAACGCGTCAACTGGGGAGATGAACCTTC | N wild type |
| | CAAAAGACGTGGTCGTTCTAACTCTCGTGGTCGGAAGAATAA | |
| | CACTATACCTCTTTCATTCTTCAATCCCATCCAACTCGAACCA | |
| | GGATCAAAATTTTGGAGCGTATGTCCGAGAGATTTTGTTCCC | |
| | AAGGGAATAGGTAACAAGGATCAACAAATTGGTTATTGGAAT | |
| | AGACAAGAGCGTTACCGCATTGTCAAAGGTCAGCGTAAGGA | |
| | ACTTCCTGAGAGGTGGTTTTTCTACTTCTTAGGCACAGGACC | |
| | TCAAGCTGATGCTAAATTTAAAGACAAGATTGATGGAGTCTT | |
| | CTGGGTTGCAAAGGATGGTGCCATGAATAAACCAACAACACT | |
| | TGGCACTCGTGGTACCAACAATGAATCCAAACCACTGAAATT | |
| | TGATGGTAAGATACCACCGCAATTTCAGCTTGAAGTGAACCG | |
| | ATCTAGGAACAACTCAAGAAGTGGTTCTCAGTCTAGATCTGT | |
| | CTCTAGAAACAGGTCTCAATCCAGAGGAAGACAACAATCCA | |
| | ATAATCAGAATAATGTTGAGGATACAATTGTAGCTGTGCTTCA | |
| | GAAATTAGGTGTTACTGAAAAGCAAAGGTCACGTTCTAAATC | |
| | TAGAGATCGTGGTGACTCTAAACCTAGAGACACAACACCTAA | |
| | TAATGCCAACAAACACACCTGGAAGAAGACTGCAGGTAAAG | |
| | GTGATGTGACAAATTTCTATGGTGCTAGAAGTGCTTCAGCTA | |
| | ACTTTGGTGATAGTGATCTCGTTGCCAACGGTAACGCTGCCA | |
| | AATCCTACCCTCAGATAGCTGAATGCGTTCCATCAGTGTCTAG | |
| | CATGCTCTTCGGTAGTCAATGGTCTGCTGAAGATGATGGTGAT | |
| | CAAGTGAAAGTCACGCTCACTCATACCTATTACCTGCCAAAA | |
| | GATGATGCCAAAACCAGCCAATTCCTAGAACAGATTGACGCT | |
| | TACAAGCGGCCATCTCAAGTGGCTAAAGATCAGAGGCAAAG | |
| | AAAATCTCGTTCTAAGTCTGCTGAGAAGAAGCCTGAGGAATT | |
| | GTCTGTAACTCTTGTAGAGGCATATACGGATGTGTTTGATGAC | |
| | ACACAGGTTGAGATGATTGATGAGGTTACGAAC | |
| 9 | GCCACACAGGGCCAGAGGGTGAACTGGGGCGACGAGCCATC | N (N2) |
| | CAAGAGGAGGGGAAGGAGCAACAGCAGAGGAAGGAAGAA | |
| | CAACACCATCCCCCTGTCCTTCTTCAACCCAATTCAGCTAGA | |
| | GCCAGGCAGCAAGTTCTGGTCAGTGTGCCCCAGAGACTTCG | |
| | TGCCCAAGGGCATCGGAAACAAGGACCAGCAGATCGGCTAC | |
| | TGGAACAGACAGGAGAGATACAGAATTGTGAAAGGCCAGAG | |
| | AAAGGAGCTGCCAGAGAGGTGGTTCTTCTACTTCCTGGGCA | |
| | CCGGCCCACAGGCAGACGCCAAGTTCAAGGACAAGATCGAT | |
| | GGAGTGTTCTGGGTGGCCAAGGACGGCGCCATGAACAAGCC | |
| | CACCACACTGGGCACAAGAGGAACAAACAATGAGAGCAAG | |
| | CCACTGAAGTTTGATGGCAAGATCCCACCCCAGTTCCAGCTG | |
| | GAGGTGAACAGGAGCAGAAACAACAGCAGAAGCGGCAGCC | |
| | AGAGCAGAAGTGTGAGCAGAAACAGAAGCCAGAGCAGAGG | |
| | AAGACAGCAGAGCAACAACCAGAACAACGTGGAGGACACC | |
| | ATCGTGGCCGTGCTGCAGAAGCTGGGGGTCACAGAAAAGCA | |
| | GAGGAGCAGAAGCAAGAGCAGGGACAGAGGAGACAGCAA | |
| | GCCAAGAGACACCACCCCCAACAACGCCAACAAGCACACCT | |
| | GGAAGAAGACAGCCGGCAAGGGAGATGTGACCAACTTCTAC | |
| | GGCGCCAGAAGCGCCAGCGCCAACTTCGGAGACTCAGACCT | |
| | GGTGGCCAATGGAAACGCAGCCAAGAGCTACCCCCAGATCG | |
| | CAGAGTGTGTGCCCTCTGTCTCCAGCATGCTGTTTGGCAGCC | |
| | AGTGGAGCGCCGAGGACGACGGTGACCAGGTGAAGGTGAC | |
| | CCTGACACACACATACTACCTGCCCAAAGATGACGCCAAGAC | |
| | CAGCCAGTTCCTGGAGCAGATTGATGCCTACAAGAGGCCCA | |
| | GCCAGGTGGCCAAGGACCAGAGACAGAGGAAGAGCAGGTC | |
| | CAAGAGCGCCGAGAAGAAGCCAGAAGAATTGAGTGTCACCC | |
| | TGGTGGAGGCCTACACAGACGTGTTTGATGACACCCAGGTG | |
| | GAGATGATTGATGAGGTGACCAAC | |
| 10 | GCCACCCAGGGCCAGAGAGTGAACTGGGGCGACGAGCCCTC | N (N3) |
| | AAAAAGGAGGGGCAGATCCAACAGCAGAGGCAGGAAGAAC | |
| | AACACCATCCCCCTGAGCTTCTTCAACCCCATCCAGCTGGAG | |
| | CCAGGCTCCAAGTTCTGGTCAGTGTGCCCAAGGGACTTCGT | |
| | GCCCAAGGGCATCGGCAACAAGGACCAGCAGATCGGCTACT | |
| | GGAACAGGCAGGAGAGATACAGAATCGTGAAGGGCCAGAG | |
| | GAAGGAACTGCCAGAAAGGTGGTTCTTCTACTTCCTGGGCA | |
| | CCGGCCCCCAGGCTGACGCCAAGTTCAAAGACAAGATCGAC | |
| | GGGGTGTTCTGGGTGGCCAAGGACGGCGCCATGAACAAGCC | |
| | AACAACACTGGGCACCAGAGGAACCAACAACGAGAGCAAG | |
| | CCACTGAAGTTTGACGGCAAGATCCCCCCCCAGTTCCAGCTG | |
| | GAAGTCAACAGGAGCAGGAACAACAGCAGGTCCGGCTCAC | |
| | AAAGCAGGAGCGTGTCCAGAAACAGATCCCAGTCAAGAGG | |
| | AAGACAGCAGTCCAACAACCAGAACAACGTGGAGGACACC | |
| | ATAGTGGCCGTGCTGCAGAAGCTGGGAGTCACAGAGAAGCA | |
| | GAGGAGCAGATCCAAGTCCAGGGACAGGGGAGACAGCAAG | |
| | CCCAGGGACACCACACCCAACAACGCCAACAAGCACACCTG | |
| | GAAGAAGACAGCCGGCAAGGGAGATGTGACCAACTTCTACG | |
| | GCGCCAGAAGCGCCTCAGCCAACTTCGGAGACTCAGACCTG | |
| | GTGGCCAACGGAAACGCCGCCAAGAGCTACCCCCAGATCGC | |
| | CGAATGTGTCCCCTCAGTGTCCTCCATGCTCTTCGGCTCACA | |
| | GTGGTCAGCAGAGGACGACGGCGACCAGGTGAAGGTGACC | |
| | CTGACCCACACCTACTACCTGCCCAAGGACGACGCCAAGAC | |
| | AAGCCAGTTCCTGGAGCAGATCGACGCCTACAAGAGGCCAT | |
| | CCCAGGTGGCCAAGGACCAGAGGCAGAGGAAGAGCAGAAG | |
| | CAAGTCAGCCGAGAAGAAACCAGAGGAGCTGTCAGTCACCC | |
| | TGGTGGAGGCCTACACCGACGTGTTCGACGACACCCAGGTG | |
| | GAGATGATCGACGAGGTGACCAAC | |
| 11 | GCAACACAAGGACAGAGAGTAAATTGGGGGGATGAGCCCAG | N (N6) |
| | CAAGAGGCGAGGCAGAAGCAACTCAAGAGGGAGAAAAAAC | |
| | AATACCATCCCACTGTCATTCTTCAACCCCATTCAACTGGAGC | |
| | CAGGCTCTAAATTCTGGAGTGTATGCCCCAGGGACTTTGTGC | |
| | CCAAGGGCATAGGGAACAAGGACCAGCAAATAGGATACTGG | |
| | AACCGGCAGGAGAGATACAGAATTGTCAAGGGTCAGAGAAA | |
| | GGAGCTGCCAGAGAGATGGTTCTTCTACTTCCTAGGAACAGG | |
| | CCCACAGGCAGACGCTAAGTTCAAGGATAAGATCGATGGTGT | |
| | CTTCTGGGTCGCCAAGGATGGTGCAATGAATAAACCAACCAC | |
| | CCTGGGGACCAGGGGGACAAACAATGAGTCCAAGCCCCTCA | |
| | AGTTTGATGGCAAAATCCCCCCACAGTTCCAGCTGGAGGTCA | |
| | ACAGGAGCAGGAACAATAGCCGTTCAGGGTCCCAGTCCAGA | |
| | TCTGTGTCCAGAAACAGGTCCCAGAGCAGGGGACGGCAGCA | |
| | GAGTAACAACCAGAATAATGTGGAAGACACCATAGTAGCAGT | |
| | GCTCCAGAAACTGGGGGTCACAGAAAAACAGAGGAGCAGG | |
| | TCCAAGTCTAGGGACCGTGGGGACTCTAAGCCAAGGGACAC | |
| | CACACCCAACAACGCCAACAAGCACACATGGAAAAAAACA | |
| | GCAGGGAAGGGTGATGTCACCAACTTTTACGGGGCCAGGTC | |
| | AGCCTCTGCAAACTTCGGGGATAGTGACCTGGTGGCCAACG | |
| | GCAATGCTGCTAAATCCTACCCTCAGATTGCTGAGTGCGTAC | |
| | CCTCTGTATCCTCTATGCTCTTTGGCTCACAATGGTCTGCTGA | |
| | GGATGATGGTGACCAGGTCAAGGTCACCTTGACCCATACCTA | |
| | CTATCTGCCCAAGGATGATGCAAAAACCAGCCAGTTCCTAGA | |
| | GCAGATAGATGCCTACAAGAGGCCCAGCCAGGTGGCCAAGG | |
| | ATCAGAGGCAGAGAAAGAGCAGATCCAAGAGCGCAGAAAA | |
| | GAAACCAGAGGAGTTATCTGTGACCCTGGTGGAGGCCTACA | |
| | CAGATGTCTTTGATGATACACAGGTGGAAATGATAGATGAGG | |
| | TGACTAAC | |
| 12 | ACAACAAATAATGAATGCATACAAGTTAACGTAACACAATTG | S wild type |
| | GCTGGCAATGAAAACCTTATCAGAGATTTTCTGTTTAGTAACT | |
| | TTAAAGAAGAAGGAAGTGTAGTTGTTGGTGGTTATTACCCTA | |
| | CAGAGGTGTGGTACAACTGCTCTAGAACAGCTCGAACTACT | |
| | GCCTTTCAGTATTTTAATAATATACATGCCTTTTATTTTGTTATG | |
| | GAAGCCATGGAAAATAGCACTGGTAATGCACGTGGTAAACC | |
| | ATTATTATTTCATGTGCATGGTGAGCCTGTTAGTGTTATTATATC | |
| | GGCTTATAGGGATGATGTGCAACAAAGGCCCCTTTTAAAACA | |
| | TGGGTTAGTGTGCATAACTAAAAATCGCCATATTAACTATGAA | |
| | CAATTCACCTCCAACCAGTGGAATTCCACATGTACGGGTGCT | |
| | GACAGAAAAATTCCTTTCTCTGTCATACCCACGGACAATGGA | |
| | ACAAAAATCTATGGTCTTGAGTGGAATGATGACTTTGTTACA | |
| | GCTTATATTAGTGGTCGTTCTTATCACTTGAACATCAATACTAA | |
| | TTGGTTTAACAATGTCACACTTTTGTATTCACGCTCAAGCACT | |
| | GCTACCTGGGAATACAGTGCTGCATATGCTTACCAAGGTGTTT | |
| | CTAACTTCACTTATTACAAGTTAAATAACACCAATGGTCTAAA | |
| | AACCTATGAATTATGTGAAGATTATGAACATTGCACTGGCTAT | |
| | GCTACCAATGTATTTGCTCCGACATCAGGTGGTTACATACCTG | |
| | ATGGATTTAGTTTTAACAATTGGTTCTTGCTTACAAATAGTTC | |
| | CACTTTTGTTAGTGGCAGGTTTGTAACAAATCAACCATTATTG | |
| | ATTAATTGCTTGTGGCCAGTGCCCAGTTTTGGTGTAGCAGCA | |
| | CAAGAATTTTGTTTTGAAGGTGCACAGTTTAGCCAATGTAAT | |
| | GGTGTGTCTTTAAATAACACAGTGGATGTTATTAGATTCAACC | |
| | TTAATTTCACTGCAGATGTACAATCTGGTATGGGTGCTACAGT | |
| | ATTTTCACTGAATACAACAGGTGGTGTCATTCTTGAAATTTCA | |
| | TGTTATAGTGACACAGTGAGTGAGTCTAGTTCTTACAGTTATG | |
| | GTGAAATCCCGTTCGGCATAACTGACGGACCACGATACTGTT | |
| | ATGTACTTTACAATGGCACAGCTCTTAAATATTTAGGAACATT | |
| | ACCACCCAGTGTAAAGGAAATTGCTATTAGTAAGTGGGGCCA | |
| | TTTTTATATTAATGGTTACAATTTCTTTAGCACATTTCCTATTGG | |
| | TTGTATATCTTTTAATTTAACCACTGGTGTTAGTGGAGCTTTTT | |
| | GGACAATTGCTTACACATCGTATACTGAAGCATTAGTACAAGT | |
| | TGAAAACACAGCTATTAAAAATGTGACGTATTGTAACAGTCA | |
| | CATTAATAACATTAAATGTTCTCAACTTACTGCTAATTTGAATA | |
| | ATGGATTTTATCCTGTTGCTTCAAGTGAAGTAGGTTTCGTTAA | |
| | TAAGAGTGTTGTGTTATTACCTAGCTTTTTCACATACACCGCT | |
| | GTCAATATAACCATTGATCTTGGTATGAAGCTTAGTGGTTATG | |
| | GTCAACCCATAGCCTCGACACTAAGTAACATCACACTACCAA | |
| | TGCAGGATAACAATACTGATGTGTACTGTATTCGTTCTAACCA | |
| | ATTCTCAGTTTATGTTCATTCCACTTGCAAAAGTTCTTTATGG | |
| | GACAATATTTTTAATCAAGACTGCACGGATGTTTTAGAGGCTA | |
| | CAGCTGTTATAAAAACTGGTACTTGTCCTTTCTCATTTGATAA | |
| | ATTGAACAATTACTTGACTTTTAACAAGTTCTGTTTGTCGTTG | |
| | AGTCCTGTTGGTGCTAATTGCAAGTTTGATGTTGCTGCACGTA | |
| | CAAGAACCAATGAGCAGGTTGTTAGAAGTCTATATGTAATATA | |
| | TGAAGAAGGAGACAACATAGTGGGTGTACCGTCTGATAATAG | |
| | CGGTCTGCACGATTTGTCTGTGCTACACCTAGACTCCTGTAC | |
| | AGATTACAATATATATGGTAGAACTGGTGTTGGTATTATTAGAC | |
| | GAACTAACAGTACGCTACTTAGTGGCTTATATTACACATCACT | |
| | ATCAGGTGATTTGTTAGGCTTTAAAAATGTTAGTGATGGTGTC | |
| | ATTTATTCTGTGACGCCATGTGATGTAAGCGCACAAGCGGCT | |
| | GTTATTGATGGTGCCATAGTTGGAGCTATGACTTCCATTAACA | |
| | GTGAACTGTTAGGTCTAACACATTGGACAACGACACCTAATT | |
| | TTTATTACTACTCTATATATAATTACACAAGTGAGAGGACTCGT | |
| | GGCACTGCAATTGACAGTAACGATGTTGATTGTGAACCTGTC | |
| | ATAACCTATTCTAATATAGGTGTTTGTAAAAATGGTGCTTTGGT | |
| | TTTTATTAACGTCACACATTCTGACGGAGACGTGCAACCAAT | |
| | TAGCACTGGTAATGTCACGATACCTACAAATTTTACTATATCTG | |
| | TGCAAGTTGAATACATGCAGGTTTACACTACACCAGTATCAAT | |
| | AGATTGTGCAAGATACGTTTGTAATGGTAACCCTAGATGTAAC | |
| | AAATTGTTAACACAATATGTGTCTGCATGTCAAACTATTGAAC | |
| | AAGCACTTGCAATGGGTGCCAGACTTGAAAACATGGAGGTT | |
| | GATTCCATGTTGTTTGTCTCGGAAAATGCCCTTAAATTGGCAT | |
| | CTGTTGAGGCGTTCAATAGTACAGAAAATTTAGATCCTATTTA | |
| | CAAAGAATGGCCTAGCATAGGTGGTTCTTGGCTAGGAGGTCT | |
| | AAAAGATATACTACCGTCCCATAATAGCAAACGTAAGTATGGT | |
| | TCTGCTATAGAAGATTTGCTTTTTGATAAAGTTGTAACATCTG | |
| | GTTTAGGTACAGTTGATGAAGATTATAAACGTTGTACTGGTGG | |
| | TTACGACATAGCAGACTTGGTGTGTGCTCAATATTACAATGGC | |
| | ATCATGGTTCTACCAGGTGTAGCTAATGCTGACAAGATGACTA | |
| | TGTACACAGCATCACTTGCAGGTGGTATAACATTAGGTGCAC | |
| | TTGGTGGTGGCGCCGTGGCTATACCTTTTGCAGTAGCAGTAC | |
| | AGGCTAGACTTAATTATGTTGCTCTACAAACTGATGTATTGAA | |
| | TAAAAACCAACAGATCCTGGCTAATGCTTTCAATCAAGCTATT | |
| | GGTAACATTACACAGGCTTTTGGTAAGGTTAATGATGCTATAC | |
| | ATCAAACATCACAAGGTCTTGCCACTGTTGCTAAAGCGTTGG | |
| | CAAAAGTGCAAGATGTTGTCAACACACAAGGGCAAGCTTTA | |
| | AGTCACCTTACAGTACAATTGCAAAATAATTTTCAAGCCATTA | |
| | GTAGTTCTATTAGTGATATTTATAACAGGCTTGACGAACTGAG | |
| | TGCTGATGCACAAGTTGATAGGCTGATTACAGGTAGACTTAC | |
| | AGCACTTAATGCATTTGTGTCTCAGACTCTAACCAGACAAGC | |
| | AGAGGTTAGGGCTAGTAGACAACTTGCCAAAGACAAGGTTA | |
| | ATGAATGTGTTAGGTCTCAGTCTCAGAGATTCGGATTCTGTGG | |
| | TAATGGTACACATTTGTTTTCACTAGCAAATGCAGCACCAAAT | |
| | GGCATGATTTTCTTTCATACAGTACTATTACCAACAGCTTATG | |
| | AAACTGTAACAGCTTGGTCAGGTATTTGTGCTTCAGATGGCG | |
| | ATCGCACTTTCGGACTTGTCGTTAAAGATGTGCAGTTGACGT | |
| | TGTTTCGTAATCTAGATGACAAGTTCTATTTGACCCCCAGAAC | |
| | TATGTATCAGCCTAGAGTTGCAACTAGTTCTGATTTTGTTCAA | |
| | ATTGAAGGGTGTGATGTGTTGTTTGTCAACGCGACTGTAATT | |
| | GATTTGCCTAGTATTATACCTGACTATATTGACATTAATCAAAC | |
| | TGTTCAAGACATATTAGAAAATTACAGACCAAACTGGACTGT | |
| | ACCTGAATTTACACTTGATATTTTCAACGCAACCTATTTAAAT | |
| | CTGACTGGTGAAATTGATGACTTAGAGTTTAGGTCAGAAAAG | |
| | CTACATAACACTACAGTAGAACTTGCCATTCTCATTGATAACA | |
| | TTAATAATACATTAGTCAATCTTGAATGGCTCAATAGAATTGA | |
| | AACTTATGTAAAATGGCCTTGGTATGTGTGGCTACTGATAGGT | |
| | TTAGTAGTAGTATTTTGCATACCATTACTGCTATTTTGCTGTTT | |
| | TAGCACAGGTTGTTGTGGATGCATAGGTTGTTTAGGAAGTTG | |
| | TTGTCACTCTATATGTAGTAGAAGACAATTTGAAAATTATGAA | |
| | CCAATTGAAAAAGTGCATGTCCACTAA | |
| 13 | ACCACCAACAATGAATGCATCCAGGTGAACGTGACCCAGCT | S (S2) |
| | GGCAGGCAATGAAAATTTGATCAGAGACTTCCTGTTCAGCAA | |
| | CTTCAAGGAGGAGGGCAGTGTAGTGGTGGGAGGCTACTACC | |
| | CAACAGAGGTGTGGTACAACTGCAGCAGAACAGCCAGAACC | |
| | ACAGCCTTCCAGTACTTCAACAACATCCACGCCTTCTACTTT | |
| | GTGATGGAGGCCATGGAAAACAGCACAGGAAATGCCAGAGG | |
| | AAAACCCCTGCTCTTCCACGTGCACGGAGAGCCCGTGTCAG | |
| | TCATCATCAGCGCCTACAGAGATGACGTCCAGCAGCGGCCCC | |
| | TGCTGAAGCATGGACTGGTCTGCATCACCAAGAACAGACAC | |
| | ATCAACTACGAGCAGTTCACCAGCAACCAGTGGAACAGCAC | |
| | CTGCACAGGAGCAGACAGAAAAATCCCCTTCAGCGTCATCC | |
| | CCACAGACAACGGCACCAAAATCTATGGCCTGGAGTGGAAT | |
| | GATGACTTTGTGACAGCCTATATCAGCGGCAGGAGCTACCAC | |
| | CTCAACATCAACACCAACTGGTTCAACAACGTCACCCTGCTC | |
| | TACTCCAGATCCAGCACAGCCACCTGGGAGTACAGCGCCGC | |
| | CTATGCCTACCAGGGAGTCTCCAACTTCACCTACTACAAACT | |
| | GAACAACACCAACGGCCTGAAAACCTACGAGCTGTGTGAGG | |
| | ACTACGAGCACTGCACAGGCTATGCCACAAATGTGTTTGCCC | |
| | CAACCAGCGGAGGCTACATCCCAGACGGCTTCTCCTTCAACA | |
| | ACTGGTTCCTCCTCACCAACTCCTCCACATTTGTGAGCGGCA | |
| | GATTTGTGACCAACCAGCCCCTGCTGATCAACTGCCTGTGGC | |
| | CCGTGCCCAGCTTTGGAGTGGCAGCCCAGGAGTTCTGCTTCG | |
| | AGGGAGCCCAGTTCAGCCAGTGCAACGGAGTCAGCCTGAAC | |
| | AACACAGTGGACGTGATCAGATTCAACCTGAACTTCACAGC | |
| | AGACGTGCAGAGTGGAATGGGAGCCACCGTCTTCAGCCTGA | |
| | ACACCACAGGAGGAGTGATCCTGGAGATCAGCTGCTACAGC | |
| | GACACAGTGAGCGAGAGCAGCAGCTACAGCTACGGAGAGAT | |
| | CCCATTTGGCATCACAGATGGCCCCAGGTACTGCTACGTCCT | |
| | GTACAATGGAACAGCCCTGAAATACCTGGGCACCCTCCCACC | |
| | CAGCGTGAAGGAGATCGCCATCAGCAAGTGGGGCCACTTCT | |
| | ACATCAATGGCTACAACTTCTTCAGCACCTTCCCCATCGGCTG | |
| | CATCTCCTTCAACCTGACCACAGGAGTGAGCGGGGCCTTCTG | |
| | GACAATCGCCTACACATCCTACACAGAAGCCCTGGTGCAGGT | |
| | GGAGAACACAGCCATCAAAAACGTCACCTACTGCAACAGCC | |
| | ACATCAACAACATCAAGTGCAGCCAGCTGACAGCCAACCTG | |
| | AACAACGGCTTCTACCCAGTGGCCAGCTCAGAGGTGGGCTT | |
| | CGTGAACAAGAGCGTGGTGCTCCTGCCCAGCTTCTTCACCTA | |
| | CACAGCAGTGAACATCACAATTGACCTGGGCATGAAGCTGA | |
| | GCGGCTACGGCCAGCCAATTGCCAGCACCCTCTCCAACATCA | |
| | CCCTCCCCATGCAGGACAATAACACAGATGTGTACTGCATCA | |
| | GATCCAACCAGTTCTCTGTCTACGTGCACAGCACCTGCAAAA | |
| | GCAGCCTGTGGGACAACATCTTCAACCAGGACTGCACAGAT | |
| | GTCCTGGAGGCCACAGCCGTGATCAAAACAGGCACCTGCCC | |
| | CTTCAGCTTTGACAAACTCAACAACTACCTTACATTCAACAA | |
| | ATTCTGCCTCTCCCTCAGCCCAGTGGGAGCCAACTGCAAGTT | |
| | TGATGTGGCCGCCAGGACCAGGACAAATGAACAAGTGGTCA | |
| | GAAGCCTCTACGTCATCTACGAGGAGGGAGACAACATCGTG | |
| | GGGGTCCCCAGCGACAACAGCGGCCTGCACGACCTGAGTGT | |
| | GCTCCACCTGGACAGCTGCACAGACTACAACATCTACGGCA | |
| | GGACTGGGGTGGGCATCATCAGAAGAACCAACAGCACACTG | |
| | CTGAGTGGCCTGTACTACACCAGCCTGAGTGGAGACTTGCTG | |
| | GGCTTCAAGAATGTGTCAGATGGGGTGATCTACAGTGTGACC | |
| | CCCTGTGACGTGTCTGCCCAGGCTGCAGTCATCGACGGAGCC | |
| | ATCGTGGGAGCCATGACCAGCATTAACAGCGAGCTGCTGGGC | |
| | CTGACCCACTGGACCACCACCCCCAACTTCTACTACTACTCC | |
| | ATCTACAACTACACATCAGAAAGAACAAGAGACACAGCCAT | |
| | CGACAGCAATGACGTGGACTGTGAGCCAGTCATCACCTACA | |
| | GCAACATCGGAGTGTGCAAGAACGGAGCCCTGGTGTTCATC | |
| | AACGTGACCCACAGCGACGGAGATGTCCAGCCCATCAGCAC | |
| | AGGAAATGTGACCATCCCAACCAACTTCACCATCAGCGTCCA | |
| | GGTGGAATACATGCAGGTGTACACCACCCCAGTGTCCATCGA | |
| | CTGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCCTGGTATGTGTGGCTGCTGATTGG | |
| | ACTGGTGGTGGTGTTCTGCATCCCACTGCTGCTGTTCTGCTG | |
| | CTTCAGCACCGGCTGCTGTGGATGCATCGGCTGCTTGGGCAG | |
| | CTGCTGCCACAGCATCTGCAGCAGGAGGCAGTTTGAGAACT | |
| | ACGAACCAATTGAAAAAGTGCACGTCCAC | |
| 14 | ACCACCAACAACGAGTGCATCCAGGTGAACGTGACCCAGCT | S (S3) |
| | GGCAGGCAACGAGAACCTCATCAGAGACTTCCTCTTCTCCA | |
| | ACTTCAAGGAGGAGGGCTCAGTGGTGGTCGGCGGCTACTAC | |
| | CCAACAGAGGTGTGGTACAACTGCTCAAGGACCGCCAGAAC | |
| | CACAGCCTTCCAGTACTTCAACAACATCCACGCCTTCTACTT | |
| | CGTGATGGAGGCCATGGAGAACTCCACCGGGAACGCCAGGG | |
| | GCAAGCCACTACTCTTCCACGTGCACGGAGAGCCAGTGAGC | |
| | GTGATCATCTCAGCCTACAGGGACGACGTGCAGCAGCGCCC | |
| | CCTGCTGAAGCATGGACTGGTGTGCATCACCAAGAACAGGC | |
| | ACATCAACTACGAGCAGTTCACCAGCAACCAGTGGAACAGC | |
| | ACCTGCACCGGCGCAGACAGGAAGATCCCCTTCTCAGTGATC | |
| | CCAACAGACAACGGAACCAAAATCTACGGCCTGGAGTGGAA | |
| | CGACGACTTCGTGACCGCCTACATCAGCGGCAGGTCCTACCA | |
| | TCTCAACATCAACACCAACTGGTTCAACAACGTCACCCTCCT | |
| | CTACAGCAGGTCATCCACAGCCACCTGGGAGTACTCAGCTGC | |
| | CTATGCATACCAGGGAGTCTCCAACTTCACATACTACAAACTC | |
| | AACAACACCAACGGCCTCAAGACCTACGAGCTGTGTGAGGA | |
| | CTACGAGCACTGCACCGGCTACGCAACAAACGTCTTCGCCCC | |
| | AACCTCCGGAGGCTACATCCCAGACGGCTTCTCCTTCAACAA | |
| | CTGGTTCCTCCTCACAAACAGCTCCACCTTCGTGTCAGGAAG | |
| | GTTCGTGACCAACCAGCCCCTGCTCATCAACTGCCTCTGGCC | |
| | CGTCCCCTCCTTCGGAGTGGCCGCCCAGGAGTTCTGCTTCGA | |
| | GGGAGCCCAGTTCTCCCAGTGCAACGGAGTCTCCCTCAACA | |
| | ACACCGTGGACGTCATCAGATTCAACCTCAACTTCACAGCAG | |
| | ACGTCCAGAGCGGCATGGGAGCCACCGTGTTCAGCCTGAAC | |
| | ACCACAGGAGGAGTGATCCTGGAGATCTCCTGCTACTCAGAC | |
| | ACAGTGTCAGAGTCCTCCTCCTACAGCTACGGAGAGATCCCA | |
| | TTCGGCATCACAGACGGCCCCAGATACTGCTACGTGCTGTAC | |
| | AACGGCACAGCCCTGAAGTACCTGGGCACCCTCCCCCCATC | |
| | AGTGAAGGAGATCGCCATCAGCAAGTGGGGCCACTTCTACAT | |
| | CAACGGCTACAACTTCTTCTCCACCTTCCCCATCGGCTGCATC | |
| | AGCTTCAACCTGACCACCGGAGTGTCCGGAGCCTTCTGGAC | |
| | CATCGCCTACACATCATACACCGAGGCCCTGGTGCAGGTGGA | |
| | GAACACAGCCATAAAGAACGTGACCTACTGCAACAGCCACA | |
| | TCAACAACATCAAGTGCTCCCAGCTGACAGCCAACCTGAAC | |
| | AACGGCTTCTACCCAGTGGCCTCCAGCGAGGTGGGCTTCGTG | |
| | AACAAGAGCGTGGTCCTACTCCCCTCCTTCTTCACCTACACA | |
| | GCAGTCAACATCACAATTGACCTGGGCATGAAGCTGTCCGGC | |
| | TACGGCCAGCCAATCGCCAGCACCCTGTCCAACATCACCCTG | |
| | CCAATGCAGGACAACAACACCGACGTCTACTGCATCAGAAG | |
| | CAACCAGTTCTCCGTGTACGTCCACTCCACCTGCAAGTCCTC | |
| | CCTCTGGGACAACATCTTCAACCAGGACTGCACAGACGTGC | |
| | TGGAGGCCACAGCTGTGATCAAGACAGGAACCTGCCCTTTC | |
| | TCATTCGACAAGCTCAACAACTACCTGACCTTCAACAAGTTC | |
| | TGCCTGAGCCTGTCCCCAGTGGGAGCCAACTGCAAGTTCGA | |
| | CGTGGCCGCCAGAACCAGGACCAACGAGCAGGTGGTCAGA | |
| | AGCCTGTACGTCATCTACGAGGAGGGAGACAACATCGTGGG | |
| | AGTGCCCAGCGACAACTCAGGCCTGCACGACCTGAGCGTGC | |
| | TGCACCTGGACTCCTGCACAGACTACAACATCTACGGCAGGA | |
| | CAGGAGTGGGCATCATCAGGAGGACCAACAGCACACTGCTG | |
| | TCCGGCCTCTACTACACCTCCCTGTCCGGAGACTTGCTGGGA | |
| | TTCAAGAACGTGTCAGACGGAGTCATCTACAGCGTCACCCCA | |
| | TGTGACGTGAGCGCCCAGGCAGCAGTGATAGACGGAGCCAT | |
| | CGTGGGAGCCATGACCTCAATCAACTCAGAACTGCTGGGCCT | |
| | CACCCACTGGACAACAACACCCAACTTCTACTACTACTCCAT | |
| | CTACAACTACACATCAGAAAGAACAAGGGACACAGCAATCG | |
| | ACTCCAACGACGTGGACTGTGAGCCAGTCATCACCTACTCCA | |
| | ACATCGGCGTGTGCAAGAACGGAGCCCTGGTGTTCATCAAC | |
| | GTCACCCACTCAGACGGCGACGTCCAGCCAATCTCCACAGG | |
| | AAACGTCACCATCCCCACCAACTTCACCATCAGCGTGCAGGT | |
| | GGAGTACATGCAGGTCTACACCACCCCAGTCTCCATCGACTG | |
| | TGCCAGGTACGTGTGCAACGGCAACCCAAGATGCAACAAAC | |
| | TGCTGACCCAGTACGTGAGCGCCTGCCAGACCATCGAGCAG | |
| | GCCCTGGCCATGGGCGCCAGGCTGGAGAACATGGAGGTGGA | |
| | CAGCATGCTCTTTGTGAGCGAGAACGCCCTGAAGCTTGCCA | |
| | GCGTGGAGGCCTTCAACAGCACCGAAAACCTGGACTCCATC | |
| | TACAAAGAGTGGCCCTCCATAGGAGGCTCCTGGCTGGGAGG | |
| | CCTGAAGGACATCCTCCCATCCCACAACAGCAAAAGAAAGT | |
| | ACGGCAGCGCCATCGAAGACCTGCTGTTCGACAAGGTGGTC | |
| | ACCTCAGGACTGGGCACAGTGGACGAGGACTACAAGAGGTG | |
| | CACCGGAGGCTACGACATCGCAGACCTGGTCTGTGCCCAGTA | |
| | CTACAACGGCATCATGGTGCTCCCAGGCGTGGCCAACGCCG | |
| | ACAAGATGACCATGTACACAGCAAGCCTGGCTGGAGGAATC | |
| | ACACTGGGAGCCCTGGGAGGAGGGGCCGTGGCCATTCCATT | |
| | CGCCGTGGCCGTGCAGGCCAGACTGAACTACGTGGCCCTGC | |
| | AGACAGACGTGCTAAACAAGAACCAGCAGATCCTGGCCAAC | |
| | GCCTTCAACCAGGCCATCGGCAACATCACCCAGGCCTTCGGC | |
| | AAGGTGAACGACGCAATCCACCAGACATCACAGGGCCTGGC | |
| | AACAGTGGCCAAGGCCCTGGCCAAGGTCCAGGACGTGGTGA | |
| | ACACCCAGGGCCAGGCCCTCTCACACCTGACAGTCCAGCTG | |
| | CAGAACAACTTCCAGGCAATCTCCTCCTCCATCTCAGACATC | |
| | TACAACAGACTGGACCCCCCCTCAGCCGACGCCCAGGTGGA | |
| | CAGACTCATCACAGGCAGGCTGACCGCCCTCAACGCCTTCGT | |
| | GTCCCAGACCCTCACCAGGCAGGCCGAGGTGAGGGCCAGCA | |
| | GGCAGCTCGCCAAGGACAAGGTGAACGAGTGCGTCAGAAG | |
| | CCAGAGCCAGAGGTTCGGCTTCTGTGGCAACGGCACCCACC | |
| | TGTTCTCCCTGGCCAACGCAGCCCCCAACGGCATGATCTTCT | |
| | TCCACACAGTCCTCCTCCCAACAGCATATGAGACAGTCACCG | |
| | CCTGGTCAGGAATCTGTGCCTCAGACGGGGACAGAACCTTC | |
| | GGCCTGGTGGTCAAGGACGTGCAGCTGACACTCTTCAGAAA | |
| | CCTGGACGACAAATTCTACCTGACCCCCAGGACCATGTACCA | |
| | GCCAAGGGTGGCCACCTCCTCAGACTTCGTGCAGATCGAGG | |
| | GCTGTGACGTGCTCTTCGTGAACGCCACCGTCATCGACCTCC | |
| | CATCCATCATCCCAGACTACATCGACATCAACCAGACAGTGC | |
| | AGGACATCCTGGAGAACTACCGCCCCAACTGGACCGTGCCA | |
| | GAGTTCACCCTAGACATATTCAACGCCACCTACCTGAACCTG | |
| | ACAGGAGAAATTGACGACCTGGAGTTCAGATCAGAAAAGCT | |
| | ACACAACACCACCGTGGAGTTAGCCATCCTCATAGACAACAT | |
| | TAACAACACCCTCGTCAACCTGGAGTGGCTCAACAGGATTG | |
| | AAACCTACGTGAAGTGGCCCTGGTACGTCTGGCTCCTCATCG | |
| | GCCTGGTGGTGGTCTTCTGCATCCCACTGCTGCTGTTCTGCT | |
| | GCTTCTCCACCGGCTGCTGTGGATGCATCGGCTGCCTGGGCT | |
| | CATGCTGCCACTCAATCTGCTCAAGGAGGCAGTTTGAAAACT | |
| | ACGAGCCAATAGAAAAAGTCCACGTCCAC | |
| 15 | ACCACAAATAACGAGTGCATTCAGGTCAACGTCACCCAGCTG | S (S6) |
| | GCCGGTAACGAGAACCTAATTAGAGACTTCCTATTCTCGAAC | |
| | TTTAAAGAGGAAGGCTCTGTGGTGGTCGGAGGTTACTACCCC | |
| | ACAGAAGTGTGGTACAATTGCTCACGTACAGCCAGGACCAC | |
| | TGCCTTCCAGTACTTCAACAACATTCATGCCTTCTACTTTGTC | |
| | ATGGAAGCCATGGAGAACTCCACTGGGAATGCCAGAGGAAA | |
| | GCCTCTCCTCTTCCATGTCCATGGAGAGCCTGTCTCTGTGATT | |
| | ATCTCAGCATATAGGGATGATGTGCAGCAGCGGCCGCTGCTT | |
| | AAGCATGGCCTAGTGTGCATTACTAAGAACCGACATATCAATT | |
| | ATGAGCAGTTCACCTCCAACCAGTGGAACTCCACATGCACTG | |
| | GTGCTGATAGGAAGATCCCGTTCAGCGTTATCCCCACCGATA | |
| | ATGGCACAAAGATTTATGGCCTAGAATGGAACGATGATTTTGT | |
| | TACTGCCTACATATCAGGAAGAAGTTACCACTTAAACATTAAC | |
| | ACCAATTGGTTCAATAATGTTACACTTCTGTACTCTCGCAGCA | |
| | GTACGGCCACTTGGGAGTATTCGGCTGCATATGCCTACCAAG | |
| | GTGTAAGCAACTTCACCTACTACAAGCTGAACAATACGAACG | |
| | GTCTGAAGACTTATGAGCTGTGCGAAGACTACGAGCACTGTA | |
| | CGGGCTATGCGACAAATGTCTTCGCCCCGACGAGCGGCGGGT | |
| | ACATACCGGATGGCTTCTCCTTCAACAACTGGTTCCTCCTTAC | |
| | CAATAGCTCCACTTTCGTATCAGGAAGATTTGTTACGAACCA | |
| | ACCCCTTCTCATTAACTGTCTGTGGCCAGTGCCCTCCTTCGGA | |
| | GTAGCTGCTCAAGAGTTCTGTTTCGAGGGTGCACAGTTCAGC | |
| | CAGTGTAATGGAGTGTCGCTGAACAACACTGTGGACGTGATC | |
| | AGGTTTAATTTGAACTTCACAGCTGATGTTCAGTCCGGCATG | |
| | GGCGCGACTGTGTTCAGCCTAAACACCACGGGTGGCGTCATC | |
| | TTGGAGATTAGTTGTTACTCTGACACTGTGTCAGAGAGCAGC | |
| | AGTTACTCCTACGGAGAAATTCCTTTCGGCATCACAGACGGT | |
| | CCCCGGTACTGCTATGTGCTGTACAACGGAACTGCTTTGAAG | |
| | TACCTGGGGACATTGCCACCTTCTGTGAAGGAAATAGCCATC | |
| | TCTAAGTGGGGTCACTTTTACATTAACGGCTATAATTTCTTTTC | |
| | CACTTTCCCAATTGGATGCATTAGCTTCAACCTGACAACAGG | |
| | TGTGTCTGGAGCCTTCTGGACCATCGCCTATACCTCTTACACA | |
| | GAGGCTCTAGTACAGGTGGAGAACACAGCTATAAAGAACGT | |
| | GACGTACTGTAACAGTCACATAAACAATATCAAGTGTTCTCA | |
| | GTTGACTGCGAACTTAAACAATGGGTTTTATCCAGTGGCGAG | |
| | CTCGGAGGTGGGGTTTGTAAACAAATCTGTGGTGCTGTTGCC | |
| | CTCCTTCTTCACGTACACTGCAGTGAACATCACCATTGATTTG | |
| | GGGATGAAACTGTCCGGCTACGGGCAGCCTATAGCATCTACA | |
| | CTGAGCAATATCACACTGCCCATGCAGGATAACAATACAGAT | |
| | GTGTACTGTATCCGCTCAAACCAGTTCTCTGTATACGTGCACA | |
| | GTACATGCAAGAGCTCGCTATGGGACAACATTTTCAACCAGG | |
| | ATTGTACTGATGTGCTTGAAGCAACTGCAGTGATCAAAACAG | |
| | GCACATGCCCGTTCAGCTTTGATAAGCTCAACAACTACCTAA | |
| | CGTTCAACAAGTTCTGCTTGAGCCTGTCTCCAGTAGGCGCCA | |
| | ATTGCAAGTTTGACGTTGCAGCGCGAACACGGACAAACGAA | |
| | CAGGTAGTGCGGTCGCTCTATGTTATCTACGAGGAGGGGGAC | |
| | AACATAGTCGGGGTTCCATCCGACAACTCAGGTTTGCACGAC | |
| | CTGAGTGTGCTCCATTTGGACTCATGCACGGATTATAACATCT | |
| | ACGGGCGCACAGGTGTGGGGATAATACGAAGAACAAACTCT | |
| | ACGCTATTGAGCGGGCTCTACTACACCTCATTGAGTGGGGAC | |
| | CTGCTAGGGTTCAAGAACGTATCTGACGGTGTGATCTATAGC | |
| | GTCACACCATGTGACGTATCAGCCCAAGCTGCTGTGATTGAC | |
| | GGGGCGATTGTGGGGGCTATGACTTCAATTAACAGCGAGCTC | |
| | CTAGGCCTGACCCACTGGACTACCACCCCAAACTTCTACTAC | |
| | TACAGCATTTATAACTATACCAGTGAGCGCACCAGGGACACT | |
| | GCCATTGACAGCAATGACGTCGACTGCGAGCCTGTTATTACC | |
| | TACAGCAACATCGGTGTTTGTAAGAATGGAGCTCTAGTCTTC | |
| | ATAAACGTAACGCACTCTGATGGCGATGTTCAACCAATTTCC | |
| | ACTGGGAACGTAACCATACCCACCAACTTTACTATTTCCGTCC | |
| | AGGTGGAGTACATGCAAGTATATACCACGCCAGTGTCCATCG | |
| | ACTGCGCTCGGTATGTGTGCAACGGTAACCCACGCTGCAATA | |
| | AGCTGCTAACGCAGTACGTCAGCGCCTGCCAGACAATAGAG | |
| | CAGGCATTGGCAATGGGTGCAAGGCTTGAAAACATGGAGGT | |
| | GGACTCCATGTTGTTCGTGTCTGAAAACGCTCTTAAACTAGC | |
| | ATCCGTGGAGGCATTCAACAGTACTGAGAACTTGGACTCTAT | |
| | CTATAAGGAGTGGCCCTCCATTGGGGGCAGCTGGCTTGGAGG | |
| | TCTAAAAGACATCCTGCCCAGCCACAACTCCAAGAGGAAGT | |
| | ACGGGTCCGCTATAGAGGACCTCCTCTTTGACAAGGTTGTTA | |
| | CTTCTGGTCTTGGCACAGTGGACGAAGACTACAAGAGGTGC | |
| | ACAGGAGGCTATGATATAGCTGACCTGGTGTGTGCTCAATACT | |
| | ACAACGGTATAATGGTTCTCCCAGGTGTGGCCAACGCTGACA | |
| | AGATGACAATGTACACAGCCTCTTTAGCTGGAGGCATTACCC | |
| | TGGGAGCCCTTGGGGGTGGCGCAGTGGCAATTCCATTTGCCG | |
| | TTGCGGTGCAGGCCCGACTAAACTATGTCGCACTTCAAACAG | |
| | ATGTGCTCAACAAGAACCAACAAATACTGGCCAACGCTTTCA | |
| | ACCAGGCCATTGGTAACATTACGCAGGCATTTGGCAAGGTGA | |
| | ATGACGCCATCCACCAGACCAGCCAGGGACTTGCCACAGTG | |
| | GCCAAGGCCTTGGCAAAGGTGCAGGATGTCGTGAACACACA | |
| | GGGTCAGGCCCTCTCTCATTTGACAGTGCAGCTTCAGAATAA | |
| | CTTCCAAGCAATCAGTTCAAGCATCAGCGACATCTACAACCG | |
| | GCTGGACCCCCCATCTGCAGATGCGCAGGTGGACAGGCTAAT | |
| | CACTGGACGCTTGACGGCACTAAATGCCTTTGTCAGCCAAAC | |
| | TCTGACCCGGCAAGCAGAGGTGCGGGCCAGTAGACAACTGG | |
| | CCAAAGACAAGGTCAACGAGTGCGTCAGGTCCCAGTCCCAG | |
| | CGTTTTGGATTCTGTGGGAACGGGACGCACCTGTTCTCATTA | |
| | GCCAATGCTGCACCCAATGGCATGATCTTTTTCCATACTGTTC | |
| | TACTTCCTACTGCCTATGAAACCGTGACCGCTTGGAGCGGCA | |
| | TCTGCGCATCTGATGGCGATAGGACCTTCGGGCTGGTCGTTA | |
| | AGGATGTCCAGCTAACGCTGTTCCGGAACTTGGATGACAAGT | |
| | TCTACCTGACCCCCAGGACCATGTACCAGCCGAGAGTGGCA | |
| | ACGAGTTCTGACTTCGTGCAAATTGAGGGCTGTGACGTCCTG | |
| | TTTGTTAATGCAACAGTGATCGATCTGCCCAGTATCATACCAG | |
| | ATTACATAGACATAAACCAGACAGTCCAGGACATACTGGAGA | |
| | ATTACAGGCCAAACTGGACCGTACCAGAGTTCACGCTGGAC | |
| | ATATTCAACGCTACGTACCTCAATTTGACTGGGGAAATTGATG | |
| | ACTTGGAGTTCAGGTCGGAGAAGCTCCACAACACCACTGTG | |
| | GAGCTGGCCATCCTGATTGACAACATCAACAACACTCTGGTG | |
| | AACCTGGAGTGGCTAAATCGCATTGAAACCTATGTCAAGTGG | |
| | CCTTGGTACGTTTGGCTACTGATCGGACTCGTGGTAGTCTTCT | |
| | GCATACCACTCCTGCTATTTTGCTGCTTCAGCACAGGGTGCTG | |
| | TGGCTGCATTGGATGCCTAGGTTCCTGCTGTCACAGTATCTGC | |
| | AGCAGAAGACAATTCGAGAACTACGAGCCCATAGAAAAGGT | |
| | CCACGTACAT | |
| 16 | MRFVMSPTVLLLLLGALAAPQTWAGS | MHC I |
| | | signal |
| | | peptide |
| 17 | ATGAGATTTGTGATGTCCCCCACTGTCCTGCTGCTGCTCCTGG | MHC I |
| | GAGCCCTGGCAGCCCCCCAGACCTGGGCAGGCAGC | signal |
| | | peptide |
| 18 | ATGAGATTTGTGATGAGCCCCACTGTGCTGCTGCTGCTGCTG | MHC I |
| | GGAGCCCTGGCAGCCCCCCAGACCTGGGCTGGCTCC | signal |
| | | peptide |
| 19 | ATGCGATTCGTCATGTCACCAACCGTTTTACTATTATTACTAGG | MHC I |
| | AGCATTAGCAGCACCGCAAACATGGGCAGGAAGT | signal |
| | | peptide |
| 20 | ATGAGATTCGTGATGTCCCCTACCGTACTACTACTCCTACTTG | MHC I |
| | GCGCACTAGCAGCTCCTCAAACTTGGGCCGGATCC | signal |
| | | peptide |
| 21 | ATGAGATTTGTGATGAGCCCCACTGTGCTGCTGCTGCTGCTG | MHC I |
| | GGAGCCCTGGCAGCCCCCCAGACCTGGGCTGGCTCA | signal |
| | | peptide |
| 22 | ATGCGTTTTGTAATGTCACCTACTGTACTACTACTACTACTCG | MHC I |
| | GAGCACTAGCAGCACCTCAGACTTGGGCCGGATCA | signal |
| | | peptide |
| 23 | FLGIIAGVVVLVVTVVVGAVIWRKKCSGRKGPSYSHAARDDST | MITD |
| | QGSDSSLMAPKV | |
| 24 | TTCCTGGGCATCATCGCAGGAGTGGTGGTGCTGGTGGTGACC | MITD |
| | GTGGTGGTGGGGGCTGTAATCTGGAGGAAGAAGTGCTCAGG | |
| | GAGAAAGGGCCCAAGCTACTCTCACGCCGCCAGGGATGACT | |
| | CCACACAGGGCTCAGACTCCTCACTGATGGCTCCAAAGGTC | |
| 25 | TTCCTGGGCATCATCGCAGGAGTGGTGGTCCTGGTGGTCACA | MITD |
| | GTCGTGGTGGGAGCAGTGATCTGGAGGAAGAAGTGCTCAGG | |
| | AAGGAAGGGCCCATCCTACTCCCACGCCGCCAGGGACGACT | |
| | CAACCCAGGGCTCAGACAGCTCCCTGATGGCCCCCAAGGTG | |
| 26 | TTCCTGGGCATCATCGCTGGCGTGGTGGTCCTGGTGGTGACT | MITD |
| | GTGGTGGTCGGAGCCGTCATCTGGAGGAAGAAGTGCAGCGG | |
| | CAGAAAGGGCCCAAGCTACAGCCACGCCGCCAGAGATGACA | |
| | GCACCCAGGGCAGCGACAGCAGCCTCATGGCCCCCAAGGTC | |
| 27 | TTCCTGGGCATCATCGCAGGAGTGGTGGTGCTGGTGGTGACA | MITD |
| | GTGGTGGTGGGAGCAGTGATCTGGAGAAAGAAATGCAGCGG | |
| | GAGAAAGGGCCCCAGCTACAGCCACGCCGCCAGGGACGAC | |
| | AGCACCCAGGGCAGCGACAGCAGCCTCATGGCCCCCAAGGT | |
| | G | |
| 28 | TTCCTGGGCATCATCGCCGGCGTGGTGGTCCTGGTGGTGACC | MITD |
| | GTGGTGGTCGGAGCAGTGATCTGGAGGAAGAAGTGCTCAGG | |
| | CAGGAAGGGCCCATCCTACAGCCACGCCGCCAGAGATGACA | |
| | GCACCCAAGGCTCAGACAGCTCCCTGATGGCCCCCAAGGTG | |
| 29 | TTCCTTGGAATCATAGCTGGGGTCGTTGTCCTCGTAGTGACTG | MITD |
| | TAGTGGTAGGCGCAGTTATCTGGAGGAAGAAATGCTCGGGG | |
| | AGGAAAGGGCCCTCTTACAGCCATGCTGCCAGGGATGACTCC | |
| | ACACAGGGGTCAGATAGCAGCCTCATGGCCCCAAAGGTC | |
| 30 | TTCCTGGGCATCATCGCCGGCGTGGTGGTCCTGGTGGTCACA | MITD |
| | GTGGTGGTGGGAGCTGTGATCTGGAGAAAGAAGTGCAGCGG | |
| | CAGGAAGGGCCCAAGCTACAGCCACGCTGCCAGAGATGACT | |
| | CCACCCAGGGCAGCGACAGCAGCCTGATGGCCCCCAAGGTG | |
| 31 | TTCCTGGGCATAATCGCCGGCGTGGTGGTGCTGGTGGTCACA | MITD |
| | GTGGTGGTCGGAGCAGTGATCTGGAGGAAGAAGTGCTCAGG | |
| | GAGGAAGGGCCCATCCTACTCCCACGCCGCCAGGGACGACA | |
| | GCACCCAGGGCTCAGACTCATCCCTGATGGCCCCCAAGGTG | |
| 32 | TTCCTGGGGATAATCGCAGGAGTGGTTGTTCTAGTGGTGACC | MITD |
| | GTGGTAGTTGGGGCAGTGATCTGGAGAAAGAAATGCTCTGG | |
| | CCGTAAGGGACCATCCTACTCCCATGCAGCACGTGATGATTC | |
| | TACCCAGGGCAGCGACAGTTCATTGATGGCCCCTAAAGTC | |
| 33 | YPYDVPDYAYPYDVPDYAYPYDVPDYA | HA tag |
| | | peptide |
| 34 | TACCCCTACGATGTGCCAGACTACGCCTACCCCTACGATGTGC | HA tag |
| | CAGACTACGCCTACCCCTACGATGTGCCAGACTACGCC | peptide |
| 35 | TACCCCTACGACGTGCCCGACTACGCCTACCCCTACGACGTG | HA tag |
| | CCAGACTACGCCTACCCCTACGACGTGCCAGACTACGCC | peptide |
| 36 | TACCCATACGATGTTCCAGACTACGCTTACCCATATGACGTGC | HA tag |
| | CAGACTATGCCTACCCCTACGACGTGCCCGACTACGCA | peptide |
| 37 | HHHHHH | His tag |
| | | peptide |
| 38 | CACCACCACCACCACCAC | His tag |
| | | peptide |
| 39 | CATCATCATCATCACCAC | His tag |
| | | peptide |
| 40 | DYKDDDDKDYKDDDDKDYKDDDDK | Flag tag |
| | | peptide |
| 41 | GACTACAAAGATGATGATGACAAGGACTACAAAGACGACGA | Flag tag |
| | CGACAAAGACTACAAGGACGATGACGACAAG | peptide |
| 42 | GACTACAAGGATGATGATGACAAGGACTACAAAGACGACGA | Flag tag |
| | CGACAAGGACTACAAGGATGACGATGACAAG | peptide |
| 43 | GACTATAAAGACGACGATGACAAAGATTATAAAGATGATGAC | Flag tag |
| | GACAAGGACTACAAGGATGATGATGACAAG | peptide |
| 44 | GGATAGCTACCAGCTTACACTTGCTTCTGACACAACTGTGTT | 5′ HBB- |
| | CGCTATCAACCACACAAACAGACGCC | UTR |
| 45 | GTGAAGCCGGGGCGATCGTCCTGAGGGCCCGGGGCCTCTTG | 3′ HBA- |
| | GGCTCCGGGCACCCGAACTTCCTGATCTTTGAATAAAGTCTG | UTR |
| | AGTGGGCTGCA | |
| 46 | RAKRGSGATNFSLLKQAGDVEENPGP | 2A linking |
| | | peptide |
| 47 | AGAGCCAAGAGAGGCAGCGGAGCCACCAACTTCAGCCTGCT | 2A linking |
| | GAAGCAGGCCGGCGACGTGGAGGAGAACCCAGGACCT | peptide |
| 48 | GGGGSGGGGSGGGGS | GS linking |
| | | peptide |
| 49 | GGAGGAGGAGGAAGCGGAGGAGGAGGAAGCGGAGGAGGA | GS linking |
| | GGAAGC | peptide |
| 50 | ATG | Start codon |
| 51 | GAGAGATACTGTGCCATGCAGAACACAGGCAGCCAGTGCAT | M_d (M2), |
| | CAATGGAACAGACAGCAGCTGCAGCACCTGCTTTGAAAGAG | amino acids |
| | GAGGCCTGATCTGGCACCTGGCCAACTGGAACTTCAGCTGG | 1 to 66 + 77 |
| | AGTGTGATCCTGATTGTCTTCATTACAGTGCTGAAGTACGGC | to 245 of M |
| | AGGCCCCAGTTCAGCTGGCTGGTGTACGGCATCATTGTGTTG | |
| | GCCCTCACCATCTTCAATGCCTACAGCGAGTACCAGGTGTCC | |
| | AGATACGTGATGTTCGGCTTCTCAGTGGCAGGAGCCGTGGTG | |
| | ACCTTTGCCCTCTGGATGATGTACTTTGTGAGGTCCATCCAGC | |
| | TCTACAGAAGAACAAAGAGCTGGTGGAGCTTCAACCCAGAA | |
| | ACCAACGCCATCCTGTGTGTCAATGCCCTGGGCAGATCCTAT | |
| | GTGCTGCCCCTGGATGGCACCCCCACAGGCGTCACCCTCACC | |
| | CTCCTGAGCGGCAACCTGTACGCCGAGGGCTTCAAGATGGC | |
| | CGGCGGCCTGACAATCGAGCACCTGCCCAAGTATGTGATGAT | |
| | TGCCACCCCCAGCAGGACAATAGTCTACACCCTGGTGGGAA | |
| | AACAGCTGAAGGCTACCACAGCCACAGGCTGGGCCTACTAC | |
| | GTCAAGAGCAAGGCCGGCGACTACAGCACAGAGGCCAGGA | |
| | CCGACAACCTGTCCGAACATGAAAAACTCCTGCACATGGTC | |
| 52 | GAGAGGTACTGCGCCATGCAGAACACAGGCTCCCAGTGCAT | M_d (M3), |
| | CAACGGAACAGACTCCTCCTGCAGCACCTGCTTCGAGAGAG | amino acids |
| | GAGGCCTCATTTGGCACCTGGCCAACTGGAACTTCAGCTGGT | 1 to 66 + 77 |
| | CAGTCATTCTGATAGTCTTCATAACAGTGCTGAAGTACGGCA | to 245 of M |
| | GGCCCCAGTTCTCCTGGCTCGTGTATGGCATCATCGTCCTGGC | |
| | CCTGACCATCTTCAACGCCTACTCAGAGTACCAGGTCAGCAG | |
| | GTACGTGATGTTCGGCTTCTCCGTGGCCGGAGCAGTGGTGAC | |
| | CTTCGCCCTGTGGATGATGTACTTCGTGAGGAGCATCCAACT | |
| | GTACAGGAGGACCAAAAGCTGGTGGTCCTTCAACCCAGAAA | |
| | CCAACGCCATCCTCTGCGTGAACGCCCTGGGCAGGTCCTACG | |
| | TCCTCCCCCTGGACGGCACCCCCACCGGGGTCACCCTCACCC | |
| | TCCTGTCAGGGAACCTGTACGCTGAGGGCTTCAAGATGGCTG | |
| | GAGGCCTGACAATTGAACACCTGCCCAAGTACGTCATGATCG | |
| | CAACACCCTCCAGAACCATCGTCTACACCCTGGTGGGCAAG | |
| | CAGCTGAAGGCCACCACCGCCACCGGCTGGGCCTACTACGT | |
| | CAAGTCCAAGGCCGGCGACTACAGCACCGAGGCCAGGACCG | |
| | ACAACCTCTCAGAGCACGAGAAGCTGCTGCACATGGTG | |
| 53 | GAGAGGTACTGTGCCATGCAGAACACCGGTTCCCAGTGCATC | M_d (M6), |
| | AACGGCACAGACTCCTCCTGCTCCACCTGCTTCGAGAGAGG | amino acids |
| | AGGCCTGATCTGGCACCTGGCAAACTGGAACTTCAGCTGGA | 1 to 66 + 77 |
| | GCGTGATCCTGATAGTGTTCATAACCGTCCTGAAGTACGGCA | to 245 of M |
| | GACCACAGTTCTCATGGCTTGTCTATGGCATCATCGTCCTGGC | |
| | CCTGACCATCTTCAACGCCTACTCTGAGTACCAGGTGTCAAG | |
| | GTATGTCATGTTCGGCTTCTCAGTGGCTGGAGCTGTGGTGAC | |
| | CTTTGCTCTGTGGATGATGTACTTCGTGAGGTCCATCCAGCTG | |
| | TACAGGAGGACAAAGTCATGGTGGTCCTTCAACCCAGAAAC | |
| | CAATGCCATCCTGTGCGTCAACGCACTGGGCAGAAGCTACGT | |
| | CCTACCACTGGACGGCACTCCTACAGGAGTGACCCTGACCCT | |
| | GCTGTCAGGCAATCTGTACGCAGAGGGGTTCAAGATGGCCG | |
| | GTGGCCTGACCATCGAGCATCTGCCTAAGTACGTGATGATCG | |
| | CCACCCCTAGCAGGACAATCGTGTACACCCTGGTGGGAAAG | |
| | CAGCTAAAGGCGACCACAGCCACAGGCTGGGCCTACTACGT | |
| | GAAGTCCAAGGCAGGGGACTATTCAACCGAGGCCAGGACCG | |
| | ACAACCTGTCAGAGCACGAGAAGCTGCTGCACATGGTC | |
| 54 | GCCACACAGGGCCAGAGGGTGAACTGGGGCGACGAGCCATC | N_d (N2), |
| | CAAGAGGAGGGGAAGGAGCAACAGCAGAGGAAGGAAGAA | amino acids |
| | CAACACCATCCCCCTGTCCTTCTTCAACCCAATTCAGCTAGA | 1 to 156 + |
| | GCCAGGCAGCAAGTTCTGGTCAGTGTGCCCCAGAGACTTCG | 167 to 375 of |
| | TGCCCAAGGGCATCGGAAACAAGGACCAGCAGATCGGCTAC | N |
| | TGGAACAGACAGGAGAGATACAGAATTGTGAAAGGCCAGAG | |
| | AAAGGAGCTGCCAGAGAGGTGGTTCTTCTACTTCCTGGGCA | |
| | CCGGCCCACAGGCAGACGCCAAGTTCAAGGACAAGATCGAT | |
| | GGAGTGTTCTGGGTGGCCAAGGACGGCGCCATGAACAAGCC | |
| | CACCACACTGGGCACAAGAGGAACAAACAATGAGAGCAAG | |
| | CCACTGAAGTTTGATGGCAAGATCCCACCCCAGTTCCAGCTG | |
| | GAGGTGAACAGGAGCAGAAGTGTGAGCAGAAACAGAAGCC | |
| | AGAGCAGAGGAAGACAGCAGAGCAACAACCAGAACAACGT | |
| | GGAGGACACCATCGTGGCCGTGCTGCAGAAGCTGGGGGTCA | |
| | CAGAAAAGCAGAGGAGCAGAAGCAAGAGCAGGGACAGAG | |
| | GAGACAGCAAGCCAAGAGACACCACCCCCAACAACGCCAA | |
| | CAAGCACACCTGGAAGAAGACAGCCGGCAAGGGAGATGTG | |
| | ACCAACTTCTACGGCGCCAGAAGCGCCAGCGCCAACTTCGG | |
| | AGACTCAGACCTGGTGGCCAATGGAAACGCAGCCAAGAGCT | |
| | ACCCCCAGATCGCAGAGTGTGTGCCCTCTGTCTCCAGCATGC | |
| | TGTTTGGCAGCCAGTGGAGCGCCGAGGACGACGGTGACCAG | |
| | GTGAAGGTGACCCTGACACACACATACTACCTGCCCAAAGAT | |
| | GACGCCAAGACCAGCCAGTTCCTGGAGCAGATTGATGCCTAC | |
| | AAGAGGCCCAGCCAGGTGGCCAAGGACCAGAGACAGAGGA | |
| | AGAGCAGGTCCAAGAGCGCCGAGAAGAAGCCAGAAGAATT | |
| | GAGTGTCACCCTGGTGGAGGCCTACACAGACGTGTTTGATGA | |
| | CACCCAGGTGGAGATGATTGATGAGGTGACCAAC | |
| 55 | GCCACCCAGGGCCAGAGAGTGAACTGGGGCGACGAGCCCTC | N_d (N3), |
| | AAAAAGGAGGGGCAGATCCAACAGCAGAGGCAGGAAGAAC | amino acids |
| | AACACCATCCCCCTGAGCTTCTTCAACCCCATCCAGCTGGAG | 1 to 156 + |
| | CCAGGCTCCAAGTTCTGGTCAGTGTGCCCAAGGGACTTCGT | 167 to 375 of |
| | GCCCAAGGGCATCGGCAACAAGGACCAGCAGATCGGCTACT | N |
| | GGAACAGGCAGGAGAGATACAGAATCGTGAAGGGCCAGAG | |
| | GAAGGAACTGCCAGAAAGGTGGTTCTTCTACTTCCTGGGCA | |
| | CCGGCCCCCAGGCTGACGCCAAGTTCAAAGACAAGATCGAC | |
| | GGGGTGTTCTGGGTGGCCAAGGACGGCGCCATGAACAAGCC | |
| | AACAACACTGGGCACCAGAGGAACCAACAACGAGAGCAAG | |
| | CCACTGAAGTTTGACGGCAAGATCCCCCCCCAGTTCCAGCTG | |
| | GAAGTCAACAGGAGCAGGAGCGTGTCCAGAAACAGATCCCA | |
| | GTCAAGAGGAAGACAGCAGTCCAACAACCAGAACAACGTG | |
| | GAGGACACCATAGTGGCCGTGCTGCAGAAGCTGGGAGTCAC | |
| | AGAGAAGCAGAGGAGCAGATCCAAGTCCAGGGACAGGGGA | |
| | GACAGCAAGCCCAGGGACACCACACCCAACAACGCCAACA | |
| | AGCACACCTGGAAGAAGACAGCCGGCAAGGGAGATGTGAC | |
| | CAACTTCTACGGCGCCAGAAGCGCCTCAGCCAACTTCGGAG | |
| | ACTCAGACCTGGTGGCCAACGGAAACGCCGCCAAGAGCTAC | |
| | CCCCAGATCGCCGAATGTGTCCCCTCAGTGTCCTCCATGCTCT | |
| | TCGGCTCACAGTGGTCAGCAGAGGACGACGGCGACCAGGTG | |
| | AAGGTGACCCTGACCCACACCTACTACCTGCCCAAGGACGA | |
| | CGCCAAGACAAGCCAGTTCCTGGAGCAGATCGACGCCTACA | |
| | AGAGGCCATCCCAGGTGGCCAAGGACCAGAGGCAGAGGAA | |
| | GAGCAGAAGCAAGTCAGCCGAGAAGAAACCAGAGGAGCTG | |
| | TCAGTCACCCTGGTGGAGGCCTACACCGACGTGTTCGACGA | |
| | CACCCAGGTGGAGATGATCGACGAGGTGACCAAC | |
| 56 | GCAACACAAGGACAGAGAGTAAATTGGGGGGATGAGCCCAG | N_d (N6), |
| | CAAGAGGCGAGGCAGAAGCAACTCAAGAGGGAGAAAAAAC | amino acids |
| | AATACCATCCCACTGTCATTCTTCAACCCCATTCAACTGGAGC | 1 to 156 + |
| | CAGGCTCTAAATTCTGGAGTGTATGCCCCAGGGACTTTGTGC | 167 to 375 of |
| | CCAAGGGCATAGGGAACAAGGACCAGCAAATAGGATACTGG | N |
| | AACCGGCAGGAGAGATACAGAATTGTCAAGGGTCAGAGAAA | |
| | GGAGCTGCCAGAGAGATGGTTCTTCTACTTCCTAGGAACAGG | |
| | CCCACAGGCAGACGCTAAGTTCAAGGATAAGATCGATGGTGT | |
| | CTTCTGGGTCGCCAAGGATGGTGCAATGAATAAACCAACCAC | |
| | CCTGGGGACCAGGGGGACAAACAATGAGTCCAAGCCCCTCA | |
| | AGTTTGATGGCAAAATCCCCCCACAGTTCCAGCTGGAGGTCA | |
| | ACAGGAGCAGGTCTGTGTCCAGAAACAGGTCCCAGAGCAGG | |
| | GGACGGCAGCAGAGTAACAACCAGAATAATGTGGAAGACAC | |
| | CATAGTAGCAGTGCTCCAGAAACTGGGGGTCACAGAAAAAC | |
| | AGAGGAGCAGGTCCAAGTCTAGGGACCGTGGGGACTCTAAG | |
| | CCAAGGGACACCACACCCAACAACGCCAACAAGCACACATG | |
| | GAAAAAAACAGCAGGGAAGGGTGATGTCACCAACTTTTACG | |
| | GGGCCAGGTCAGCCTCTGCAAACTTCGGGGATAGTGACCTG | |
| | GTGGCCAACGGCAATGCTGCTAAATCCTACCCTCAGATTGCT | |
| | GAGTGCGTACCCTCTGTATCCTCTATGCTCTTTGGCTCACAAT | |
| | GGTCTGCTGAGGATGATGGTGACCAGGTCAAGGTCACCTTG | |
| | ACCCATACCTACTATCTGCCCAAGGATGATGCAAAAACCAGC | |
| | CAGTTCCTAGAGCAGATAGATGCCTACAAGAGGCCCAGCCA | |
| | GGTGGCCAAGGATCAGAGGCAGAGAAAGAGCAGATCCAAG | |
| | AGCGCAGAAAAGAAACCAGAGGAGTTATCTGTGACCCTGGT | |
| | GGAGGCCTACACAGATGTCTTTGATGATACACAGGTGGAAAT | |
| | GATAGATGAGGTGACTAAC | |
| 57 | ACCACCAACAATGAATGCATCCAGGTGAACGTGACCCAGCT | S_ec (S2), |
| | GGCAGGCAATGAAAATTTGATCAGAGACTTCCTGTTCAGCAA | amino acids |
| | CTTCAAGGAGGAGGGCAGTGTAGTGGTGGGAGGCTACTACC | 1 to 1374 of |
| | CAACAGAGGTGTGGTACAACTGCAGCAGAACAGCCAGAACC | S |
| | ACAGCCTTCCAGTACTTCAACAACATCCACGCCTTCTACTTT | |
| | GTGATGGAGGCCATGGAAAACAGCACAGGAAATGCCAGAGG | |
| | AAAACCCCTGCTCTTCCACGTGCACGGAGAGCCCGTGTCAG | |
| | TCATCATCAGCGCCTACAGAGATGACGTCCAGCAGCGGCCCC | |
| | TGCTGAAGCATGGACTGGTCTGCATCACCAAGAACAGACAC | |
| | ATCAACTACGAGCAGTTCACCAGCAACCAGTGGAACAGCAC | |
| | CTGCACAGGAGCAGACAGAAAAATCCCCTTCAGCGTCATCC | |
| | CCACAGACAACGGCACCAAAATCTATGGCCTGGAGTGGAAT | |
| | GATGACTTTGTGACAGCCTATATCAGCGGCAGGAGCTACCAC | |
| | CTCAACATCAACACCAACTGGTTCAACAACGTCACCCTGCTC | |
| | TACTCCAGATCCAGCACAGCCACCTGGGAGTACAGCGCCGC | |
| | CTATGCCTACCAGGGAGTCTCCAACTTCACCTACTACAAACT | |
| | GAACAACACCAACGGCCTGAAAACCTACGAGCTGTGTGAGG | |
| | ACTACGAGCACTGCACAGGCTATGCCACAAATGTGTTTGCCC | |
| | CAACCAGCGGAGGCTACATCCCAGACGGCTTCTCCTTCAACA | |
| | ACTGGTTCCTCCTCACCAACTCCTCCACATTTGTGAGCGGCA | |
| | GATTTGTGACCAACCAGCCCCTGCTGATCAACTGCCTGTGGC | |
| | CCGTGCCCAGCTTTGGAGTGGCAGCCCAGGAGTTCTGCTTCG | |
| | AGGGAGCCCAGTTCAGCCAGTGCAACGGAGTCAGCCTGAAC | |
| | AACACAGTGGACGTGATCAGATTCAACCTGAACTTCACAGC | |
| | AGACGTGCAGAGTGGAATGGGAGCCACCGTCTTCAGCCTGA | |
| | ACACCACAGGAGGAGTGATCCTGGAGATCAGCTGCTACAGC | |
| | GACACAGTGAGCGAGAGCAGCAGCTACAGCTACGGAGAGAT | |
| | CCCATTTGGCATCACAGATGGCCCCAGGTACTGCTACGTCCT | |
| | GTACAATGGAACAGCCCTGAAATACCTGGGCACCCTCCCACC | |
| | CAGCGTGAAGGAGATCGCCATCAGCAAGTGGGGCCACTTCT | |
| | ACATCAATGGCTACAACTTCTTCAGCACCTTCCCCATCGGCTG | |
| | CATCTCCTTCAACCTGACCACAGGAGTGAGCGGGGCCTTCTG | |
| | GACAATCGCCTACACATCCTACACAGAAGCCCTGGTGCAGGT | |
| | GGAGAACACAGCCATCAAAAACGTCACCTACTGCAACAGCC | |
| | ACATCAACAACATCAAGTGCAGCCAGCTGACAGCCAACCTG | |
| | AACAACGGCTTCTACCCAGTGGCCAGCTCAGAGGTGGGCTT | |
| | CGTGAACAAGAGCGTGGTGCTCCTGCCCAGCTTCTTCACCTA | |
| | CACAGCAGTGAACATCACAATTGACCTGGGCATGAAGCTGA | |
| | GCGGCTACGGCCAGCCAATTGCCAGCACCCTCTCCAACATCA | |
| | CCCTCCCCATGCAGGACAATAACACAGATGTGTACTGCATCA | |
| | GATCCAACCAGTTCTCTGTCTACGTGCACAGCACCTGCAAAA | |
| | GCAGCCTGTGGGACAACATCTTCAACCAGGACTGCACAGAT | |
| | GTCCTGGAGGCCACAGCCGTGATCAAAACAGGCACCTGCCC | |
| | CTTCAGCTTTGACAAACTCAACAACTACCTTACATTCAACAA | |
| | ATTCTGCCTCTCCCTCAGCCCAGTGGGAGCCAACTGCAAGTT | |
| | TGATGTGGCCGCCAGGACCAGGACAAATGAACAAGTGGTCA | |
| | GAAGCCTCTACGTCATCTACGAGGAGGGAGACAACATCGTG | |
| | GGGGTCCCCAGCGACAACAGCGGCCTGCACGACCTGAGTGT | |
| | GCTCCACCTGGACAGCTGCACAGACTACAACATCTACGGCA | |
| | GGACTGGGGTGGGCATCATCAGAAGAACCAACAGCACACTG | |
| | CTGAGTGGCCTGTACTACACCAGCCTGAGTGGAGACTTGCTG | |
| | GGCTTCAAGAATGTGTCAGATGGGGTGATCTACAGTGTGACC | |
| | CCCTGTGACGTGTCTGCCCAGGCTGCAGTCATCGACGGAGCC | |
| | ATCGTGGGAGCCATGACCAGCATTAACAGCGAGCTGCTGGGC | |
| | CTGACCCACTGGACCACCACCCCCAACTTCTACTACTACTCC | |
| | ATCTACAACTACACATCAGAAAGAACAAGAGACACAGCCAT | |
| | CGACAGCAATGACGTGGACTGTGAGCCAGTCATCACCTACA | |
| | GCAACATCGGAGTGTGCAAGAACGGAGCCCTGGTGTTCATC | |
| | AACGTGACCCACAGCGACGGAGATGTCCAGCCCATCAGCAC | |
| | AGGAAATGTGACCATCCCAACCAACTTCACCATCAGCGTCCA | |
| | GGTGGAATACATGCAGGTGTACACCACCCCAGTGTCCATCGA | |
| | CTGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCC | |
| 58 | ACCACCAACAACGAGTGCATCCAGGTGAACGTGACCCAGCT | S_ec (S3), |
| | GGCAGGCAACGAGAACCTCATCAGAGACTTCCTCTTCTCCA | amino acids |
| | ACTTCAAGGAGGAGGGCTCAGTGGTGGTCGGCGGCTACTAC | 1 to 1374 of |
| | CCAACAGAGGTGTGGTACAACTGCTCAAGGACCGCCAGAAC | S |
| | CACAGCCTTCCAGTACTTCAACAACATCCACGCCTTCTACTT | |
| | CGTGATGGAGGCCATGGAGAACTCCACCGGGAACGCCAGGG | |
| | GCAAGCCACTACTCTTCCACGTGCACGGAGAGCCAGTGAGC | |
| | GTGATCATCTCAGCCTACAGGGACGACGTGCAGCAGCGCCC | |
| | CCTGCTGAAGCATGGACTGGTGTGCATCACCAAGAACAGGC | |
| | ACATCAACTACGAGCAGTTCACCAGCAACCAGTGGAACAGC | |
| | ACCTGCACCGGCGCAGACAGGAAGATCCCCTTCTCAGTGATC | |
| | CCAACAGACAACGGAACCAAAATCTACGGCCTGGAGTGGAA | |
| | CGACGACTTCGTGACCGCCTACATCAGCGGCAGGTCCTACCA | |
| | TCTCAACATCAACACCAACTGGTTCAACAACGTCACCCTCCT | |
| | CTACAGCAGGTCATCCACAGCCACCTGGGAGTACTCAGCTGC | |
| | CTATGCATACCAGGGAGTCTCCAACTTCACATACTACAAACTC | |
| | AACAACACCAACGGCCTCAAGACCTACGAGCTGTGTGAGGA | |
| | CTACGAGCACTGCACCGGCTACGCAACAAACGTCTTCGCCCC | |
| | AACCTCCGGAGGCTACATCCCAGACGGCTTCTCCTTCAACAA | |
| | CTGGTTCCTCCTCACAAACAGCTCCACCTTCGTGTCAGGAAG | |
| | GTTCGTGACCAACCAGCCCCTGCTCATCAACTGCCTCTGGCC | |
| | CGTCCCCTCCTTCGGAGTGGCCGCCCAGGAGTTCTGCTTCGA | |
| | GGGAGCCCAGTTCTCCCAGTGCAACGGAGTCTCCCTCAACA | |
| | ACACCGTGGACGTCATCAGATTCAACCTCAACTTCACAGCAG | |
| | ACGTCCAGAGCGGCATGGGAGCCACCGTGTTCAGCCTGAAC | |
| | ACCACAGGAGGAGTGATCCTGGAGATCTCCTGCTACTCAGAC | |
| | ACAGTGTCAGAGTCCTCCTCCTACAGCTACGGAGAGATCCCA | |
| | TTCGGCATCACAGACGGCCCCAGATACTGCTACGTGCTGTAC | |
| | AACGGCACAGCCCTGAAGTACCTGGGCACCCTCCCCCCATC | |
| | AGTGAAGGAGATCGCCATCAGCAAGTGGGGCCACTTCTACAT | |
| | CAACGGCTACAACTTCTTCTCCACCTTCCCCATCGGCTGCATC | |
| | AGCTTCAACCTGACCACCGGAGTGTCCGGAGCCTTCTGGAC | |
| | CATCGCCTACACATCATACACCGAGGCCCTGGTGCAGGTGGA | |
| | GAACACAGCCATAAAGAACGTGACCTACTGCAACAGCCACA | |
| | TCAACAACATCAAGTGCTCCCAGCTGACAGCCAACCTGAAC | |
| | AACGGCTTCTACCCAGTGGCCTCCAGCGAGGTGGGCTTCGTG | |
| | AACAAGAGCGTGGTCCTACTCCCCTCCTTCTTCACCTACACA | |
| | GCAGTCAACATCACAATTGACCTGGGCATGAAGCTGTCCGGC | |
| | TACGGCCAGCCAATCGCCAGCACCCTGTCCAACATCACCCTG | |
| | CCAATGCAGGACAACAACACCGACGTCTACTGCATCAGAAG | |
| | CAACCAGTTCTCCGTGTACGTCCACTCCACCTGCAAGTCCTC | |
| | CCTCTGGGACAACATCTTCAACCAGGACTGCACAGACGTGC | |
| | TGGAGGCCACAGCTGTGATCAAGACAGGAACCTGCCCTTTC | |
| | TCATTCGACAAGCTCAACAACTACCTGACCTTCAACAAGTTC | |
| | TGCCTGAGCCTGTCCCCAGTGGGAGCCAACTGCAAGTTCGA | |
| | CGTGGCCGCCAGAACCAGGACCAACGAGCAGGTGGTCAGA | |
| | AGCCTGTACGTCATCTACGAGGAGGGAGACAACATCGTGGG | |
| | AGTGCCCAGCGACAACTCAGGCCTGCACGACCTGAGCGTGC | |
| | TGCACCTGGACTCCTGCACAGACTACAACATCTACGGCAGGA | |
| | CAGGAGTGGGCATCATCAGGAGGACCAACAGCACACTGCTG | |
| | TCCGGCCTCTACTACACCTCCCTGTCCGGAGACTTGCTGGGA | |
| | TTCAAGAACGTGTCAGACGGAGTCATCTACAGCGTCACCCCA | |
| | TGTGACGTGAGCGCCCAGGCAGCAGTGATAGACGGAGCCAT | |
| | CGTGGGAGCCATGACCTCAATCAACTCAGAACTGCTGGGCCT | |
| | CACCCACTGGACAACAACACCCAACTTCTACTACTACTCCAT | |
| | CTACAACTACACATCAGAAAGAACAAGGGACACAGCAATCG | |
| | ACTCCAACGACGTGGACTGTGAGCCAGTCATCACCTACTCCA | |
| | ACATCGGCGTGTGCAAGAACGGAGCCCTGGTGTTCATCAAC | |
| | GTCACCCACTCAGACGGCGACGTCCAGCCAATCTCCACAGG | |
| | AAACGTCACCATCCCCACCAACTTCACCATCAGCGTGCAGGT | |
| | GGAGTACATGCAGGTCTACACCACCCCAGTCTCCATCGACTG | |
| | TGCCAGGTACGTGTGCAACGGCAACCCAAGATGCAACAAAC | |
| | TGCTGACCCAGTACGTGAGCGCCTGCCAGACCATCGAGCAG | |
| | GCCCTGGCCATGGGCGCCAGGCTGGAGAACATGGAGGTGGA | |
| | CAGCATGCTCTTTGTGAGCGAGAACGCCCTGAAGCTTGCCA | |
| | GCGTGGAGGCCTTCAACAGCACCGAAAACCTGGACTCCATC | |
| | TACAAAGAGTGGCCCTCCATAGGAGGCTCCTGGCTGGGAGG | |
| | CCTGAAGGACATCCTCCCATCCCACAACAGCAAAAGAAAGT | |
| | ACGGCAGCGCCATCGAAGACCTGCTGTTCGACAAGGTGGTC | |
| | ACCTCAGGACTGGGCACAGTGGACGAGGACTACAAGAGGTG | |
| | CACCGGAGGCTACGACATCGCAGACCTGGTCTGTGCCCAGTA | |
| | CTACAACGGCATCATGGTGCTCCCAGGCGTGGCCAACGCCG | |
| | ACAAGATGACCATGTACACAGCAAGCCTGGCTGGAGGAATC | |
| | ACACTGGGAGCCCTGGGAGGAGGGGCCGTGGCCATTCCATT | |
| | CGCCGTGGCCGTGCAGGCCAGACTGAACTACGTGGCCCTGC | |
| | AGACAGACGTGCTAAACAAGAACCAGCAGATCCTGGCCAAC | |
| | GCCTTCAACCAGGCCATCGGCAACATCACCCAGGCCTTCGGC | |
| | AAGGTGAACGACGCAATCCACCAGACATCACAGGGCCTGGC | |
| | AACAGTGGCCAAGGCCCTGGCCAAGGTCCAGGACGTGGTGA | |
| | ACACCCAGGGCCAGGCCCTCTCACACCTGACAGTCCAGCTG | |
| | CAGAACAACTTCCAGGCAATCTCCTCCTCCATCTCAGACATC | |
| | TACAACAGACTGGACCCCCCCTCAGCCGACGCCCAGGTGGA | |
| | CAGACTCATCACAGGCAGGCTGACCGCCCTCAACGCCTTCGT | |
| | GTCCCAGACCCTCACCAGGCAGGCCGAGGTGAGGGCCAGCA | |
| | GGCAGCTCGCCAAGGACAAGGTGAACGAGTGCGTCAGAAG | |
| | CCAGAGCCAGAGGTTCGGCTTCTGTGGCAACGGCACCCACC | |
| | TGTTCTCCCTGGCCAACGCAGCCCCCAACGGCATGATCTTCT | |
| | TCCACACAGTCCTCCTCCCAACAGCATATGAGACAGTCACCG | |
| | CCTGGTCAGGAATCTGTGCCTCAGACGGGGACAGAACCTTC | |
| | GGCCTGGTGGTCAAGGACGTGCAGCTGACACTCTTCAGAAA | |
| | CCTGGACGACAAATTCTACCTGACCCCCAGGACCATGTACCA | |
| | GCCAAGGGTGGCCACCTCCTCAGACTTCGTGCAGATCGAGG | |
| | GCTGTGACGTGCTCTTCGTGAACGCCACCGTCATCGACCTCC | |
| | CATCCATCATCCCAGACTACATCGACATCAACCAGACAGTGC | |
| | AGGACATCCTGGAGAACTACCGCCCCAACTGGACCGTGCCA | |
| | GAGTTCACCCTAGACATATTCAACGCCACCTACCTGAACCTG | |
| | ACAGGAGAAATTGACGACCTGGAGTTCAGATCAGAAAAGCT | |
| | ACACAACACCACCGTGGAGTTAGCCATCCTCATAGACAACAT | |
| | TAACAACACCCTCGTCAACCTGGAGTGGCTCAACAGGATTG | |
| | AAACCTACGTGAAGTGGCCC | |
| 59 | ACCACAAATAACGAGTGCATTCAGGTCAACGTCACCCAGCTG | S_ec (S6), |
| | GCCGGTAACGAGAACCTAATTAGAGACTTCCTATTCTCGAAC | amino acids |
| | TTTAAAGAGGAAGGCTCTGTGGTGGTCGGAGGTTACTACCCC | 1 to 1374 of |
| | ACAGAAGTGTGGTACAATTGCTCACGTACAGCCAGGACCAC | S |
| | TGCCTTCCAGTACTTCAACAACATTCATGCCTTCTACTTTGTC | |
| | ATGGAAGCCATGGAGAACTCCACTGGGAATGCCAGAGGAAA | |
| | GCCTCTCCTCTTCCATGTCCATGGAGAGCCTGTCTCTGTGATT | |
| | ATCTCAGCATATAGGGATGATGTGCAGCAGCGGCCGCTGCTT | |
| | AAGCATGGCCTAGTGTGCATTACTAAGAACCGACATATCAATT | |
| | ATGAGCAGTTCACCTCCAACCAGTGGAACTCCACATGCACTG | |
| | GTGCTGATAGGAAGATCCCGTTCAGCGTTATCCCCACCGATA | |
| | ATGGCACAAAGATTTATGGCCTAGAATGGAACGATGATTTTGT | |
| | TACTGCCTACATATCAGGAAGAAGTTACCACTTAAACATTAAC | |
| | ACCAATTGGTTCAATAATGTTACACTTCTGTACTCTCGCAGCA | |
| | GTACGGCCACTTGGGAGTATTCGGCTGCATATGCCTACCAAG | |
| | GTGTAAGCAACTTCACCTACTACAAGCTGAACAATACGAACG | |
| | GTCTGAAGACTTATGAGCTGTGCGAAGACTACGAGCACTGTA | |
| | CGGGCTATGCGACAAATGTCTTCGCCCCGACGAGCGGCGGGT | |
| | ACATACCGGATGGCTTCTCCTTCAACAACTGGTTCCTCCTTAC | |
| | CAATAGCTCCACTTTCGTATCAGGAAGATTTGTTACGAACCA | |
| | ACCCCTTCTCATTAACTGTCTGTGGCCAGTGCCCTCCTTCGGA | |
| | GTAGCTGCTCAAGAGTTCTGTTTCGAGGGTGCACAGTTCAGC | |
| | CAGTGTAATGGAGTGTCGCTGAACAACACTGTGGACGTGATC | |
| | AGGTTTAATTTGAACTTCACAGCTGATGTTCAGTCCGGCATG | |
| | GGCGCGACTGTGTTCAGCCTAAACACCACGGGTGGCGTCATC | |
| | TTGGAGATTAGTTGTTACTCTGACACTGTGTCAGAGAGCAGC | |
| | AGTTACTCCTACGGAGAAATTCCTTTCGGCATCACAGACGGT | |
| | CCCCGGTACTGCTATGTGCTGTACAACGGAACTGCTTTGAAG | |
| | TACCTGGGGACATTGCCACCTTCTGTGAAGGAAATAGCCATC | |
| | TCTAAGTGGGGTCACTTTTACATTAACGGCTATAATTTCTTTTC | |
| | CACTTTCCCAATTGGATGCATTAGCTTCAACCTGACAACAGG | |
| | TGTGTCTGGAGCCTTCTGGACCATCGCCTATACCTCTTACACA | |
| | GAGGCTCTAGTACAGGTGGAGAACACAGCTATAAAGAACGT | |
| | GACGTACTGTAACAGTCACATAAACAATATCAAGTGTTCTCA | |
| | GTTGACTGCGAACTTAAACAATGGGTTTTATCCAGTGGCGAG | |
| | CTCGGAGGTGGGGTTTGTAAACAAATCTGTGGTGCTGTTGCC | |
| | CTCCTTCTTCACGTACACTGCAGTGAACATCACCATTGATTTG | |
| | GGGATGAAACTGTCCGGCTACGGGCAGCCTATAGCATCTACA | |
| | CTGAGCAATATCACACTGCCCATGCAGGATAACAATACAGAT | |
| | GTGTACTGTATCCGCTCAAACCAGTTCTCTGTATACGTGCACA | |
| | GTACATGCAAGAGCTCGCTATGGGACAACATTTTCAACCAGG | |
| | ATTGTACTGATGTGCTTGAAGCAACTGCAGTGATCAAAACAG | |
| | GCACATGCCCGTTCAGCTTTGATAAGCTCAACAACTACCTAA | |
| | CGTTCAACAAGTTCTGCTTGAGCCTGTCTCCAGTAGGCGCCA | |
| | ATTGCAAGTTTGACGTTGCAGCGCGAACACGGACAAACGAA | |
| | CAGGTAGTGCGGTCGCTCTATGTTATCTACGAGGAGGGGGAC | |
| | AACATAGTCGGGGTTCCATCCGACAACTCAGGTTTGCACGAC | |
| | CTGAGTGTGCTCCATTTGGACTCATGCACGGATTATAACATCT | |
| | ACGGGCGCACAGGTGTGGGGATAATACGAAGAACAAACTCT | |
| | ACGCTATTGAGCGGGCTCTACTACACCTCATTGAGTGGGGAC | |
| | CTGCTAGGGTTCAAGAACGTATCTGACGGTGTGATCTATAGC | |
| | GTCACACCATGTGACGTATCAGCCCAAGCTGCTGTGATTGAC | |
| | GGGGCGATTGTGGGGGCTATGACTTCAATTAACAGCGAGCTC | |
| | CTAGGCCTGACCCACTGGACTACCACCCCAAACTTCTACTAC | |
| | TACAGCATTTATAACTATACCAGTGAGCGCACCAGGGACACT | |
| | GCCATTGACAGCAATGACGTCGACTGCGAGCCTGTTATTACC | |
| | TACAGCAACATCGGTGTTTGTAAGAATGGAGCTCTAGTCTTC | |
| | ATAAACGTAACGCACTCTGATGGCGATGTTCAACCAATTTCC | |
| | ACTGGGAACGTAACCATACCCACCAACTTTACTATTTCCGTCC | |
| | AGGTGGAGTACATGCAAGTATATACCACGCCAGTGTCCATCG | |
| | ACTGCGCTCGGTATGTGTGCAACGGTAACCCACGCTGCAATA | |
| | AGCTGCTAACGCAGTACGTCAGCGCCTGCCAGACAATAGAG | |
| | CAGGCATTGGCAATGGGTGCAAGGCTTGAAAACATGGAGGT | |
| | GGACTCCATGTTGTTCGTGTCTGAAAACGCTCTTAAACTAGC | |
| | ATCCGTGGAGGCATTCAACAGTACTGAGAACTTGGACTCTAT | |
| | CTATAAGGAGTGGCCCTCCATTGGGGGCAGCTGGCTTGGAGG | |
| | TCTAAAAGACATCCTGCCCAGCCACAACTCCAAGAGGAAGT | |
| | ACGGGTCCGCTATAGAGGACCTCCTCTTTGACAAGGTTGTTA | |
| | CTTCTGGTCTTGGCACAGTGGACGAAGACTACAAGAGGTGC | |
| | ACAGGAGGCTATGATATAGCTGACCTGGTGTGTGCTCAATACT | |
| | ACAACGGTATAATGGTTCTCCCAGGTGTGGCCAACGCTGACA | |
| | AGATGACAATGTACACAGCCTCTTTAGCTGGAGGCATTACCC | |
| | TGGGAGCCCTTGGGGGTGGCGCAGTGGCAATTCCATTTGCCG | |
| | TTGCGGTGCAGGCCCGACTAAACTATGTCGCACTTCAAACAG | |
| | ATGTGCTCAACAAGAACCAACAAATACTGGCCAACGCTTTCA | |
| | ACCAGGCCATTGGTAACATTACGCAGGCATTTGGCAAGGTGA | |
| | ATGACGCCATCCACCAGACCAGCCAGGGACTTGCCACAGTG | |
| | GCCAAGGCCTTGGCAAAGGTGCAGGATGTCGTGAACACACA | |
| | GGGTCAGGCCCTCTCTCATTTGACAGTGCAGCTTCAGAATAA | |
| | CTTCCAAGCAATCAGTTCAAGCATCAGCGACATCTACAACCG | |
| | GCTGGACCCCCCATCTGCAGATGCGCAGGTGGACAGGCTAAT | |
| | CACTGGACGCTTGACGGCACTAAATGCCTTTGTCAGCCAAAC | |
| | TCTGACCCGGCAAGCAGAGGTGCGGGCCAGTAGACAACTGG | |
| | CCAAAGACAAGGTCAACGAGTGCGTCAGGTCCCAGTCCCAG | |
| | CGTTTTGGATTCTGTGGGAACGGGACGCACCTGTTCTCATTA | |
| | GCCAATGCTGCACCCAATGGCATGATCTTTTTCCATACTGTTC | |
| | TACTTCCTACTGCCTATGAAACCGTGACCGCTTGGAGCGGCA | |
| | TCTGCGCATCTGATGGCGATAGGACCTTCGGGCTGGTCGTTA | |
| | AGGATGTCCAGCTAACGCTGTTCCGGAACTTGGATGACAAGT | |
| | TCTACCTGACCCCCAGGACCATGTACCAGCCGAGAGTGGCA | |
| | ACGAGTTCTGACTTCGTGCAAATTGAGGGCTGTGACGTCCTG | |
| | TTTGTTAATGCAACAGTGATCGATCTGCCCAGTATCATACCAG | |
| | ATTACATAGACATAAACCAGACAGTCCAGGACATACTGGAGA | |
| | ATTACAGGCCAAACTGGACCGTACCAGAGTTCACGCTGGAC | |
| | ATATTCAACGCTACGTACCTCAATTTGACTGGGGAAATTGATG | |
| | ACTTGGAGTTCAGGTCGGAGAAGCTCCACAACACCACTGTG | |
| | GAGCTGGCCATCCTGATTGACAACATCAACAACACTCTGGTG | |
| | AACCTGGAGTGGCTAAATCGCATTGAAACCTATGTCAAGTGG | |
| | CCT | |
| 60 | TGTGAGCCAGTCATCACCTACAGCAACATCGGAGTGTGCAAG | SII (S2), |
| | AACGGAGCCCTGGTGTTCATCAACGTGACCCACAGCGACGG | amino acids |
| | AGATGTCCAGCCCATCAGCACAGGAAATGTGACCATCCCAAC | 782 to 1433 |
| | CAACTTCACCATCAGCGTCCAGGTGGAATACATGCAGGTGTA | of S |
| | CACCACCCCAGTGTCCATCGACTGTGCCAGATACGTGTGCAA | |
| | TGGAAACCCCAGATGCAACAAGCTCCTCACCCAGTACGTGTC | |
| | AGCCTGCCAGACAATCGAGCAGGCCCTGGCCATGGGAGCCA | |
| | GGCTCGAGAACATGGAAGTGGACAGCATGCTGTTTGTCTCA | |
| | GAGAATGCCCTGAAACTGGCCAGCGTGGAGGCCTTCAACAG | |
| | CACAGAGAACCTGGACAGCATCTACAAGGAGTGGCCATCAA | |
| | TCGGAGGCAGCTGGCTGGGAGGACTTAAGGACATCCTGCCA | |
| | AGCCACAACAGCAAAAGAAAGTACGGCAGCGCCATTGAGGA | |
| | CCTGCTGTTTGACAAGGTGGTCACCTCCGGCCTGGGCACAGT | |
| | GGATGAGGACTACAAGAGATGCACCGGCGGCTATGACATTGC | |
| | CGACCTGGTGTGTGCCCAGTACTACAATGGCATCATGGTGCT | |
| | GCCTGGAGTGGCCAACGCCGACAAAATGACCATGTACACCG | |
| | CCTCCCTGGCTGGAGGCATCACACTGGGAGCCCTGGGGGGA | |
| | GGAGCAGTGGCCATCCCCTTTGCAGTGGCTGTGCAGGCCAG | |
| | ACTCAACTACGTGGCCCTGCAGACAGACGTGCTCAACAAGA | |
| | ACCAGCAGATCCTGGCCAACGCTTTCAACCAGGCTATCGGAA | |
| | ACATCACCCAGGCCTTTGGAAAAGTGAATGATGCCATCCACC | |
| | AGACCAGCCAGGGCCTGGCCACAGTGGCCAAGGCCCTGGCC | |
| | AAGGTGCAGGACGTGGTCAACACCCAGGGCCAGGCCCTCAG | |
| | TCACCTCACAGTACAGCTCCAGAACAACTTCCAGGCAATCTC | |
| | CTCCTCCATCAGCGACATCTACAACAGGCTGGACCCCCCAAG | |
| | CGCTGATGCCCAGGTGGACAGACTGATCACAGGAAGACTCA | |
| | CAGCCCTCAACGCATTTGTGTCCCAGACACTGACCAGGCAG | |
| | GCAGAGGTCAGGGCCAGCAGGCAGCTGGCCAAGGACAAGG | |
| | TGAATGAGTGCGTGAGGAGCCAGAGCCAGAGATTTGGCTTC | |
| | TGCGGAAACGGCACCCACCTGTTCAGCCTGGCCAACGCCGC | |
| | CCCCAACGGCATGATTTTCTTCCACACAGTCCTCCTCCCCAC | |
| | AGCCTACGAAACAGTGACAGCCTGGTCAGGCATCTGTGCCA | |
| | GCGACGGAGACAGAACCTTTGGCCTGGTGGTGAAGGATGTG | |
| | CAGCTCACCCTCTTCAGAAACCTGGATGACAAGTTCTACCTC | |
| | ACCCCAAGAACCATGTACCAGCCCAGAGTGGCCACAAGCAG | |
| | CGACTTTGTGCAGATTGAGGGCTGTGACGTGCTGTTTGTGAA | |
| | TGCAACAGTGATTGACCTCCCAAGCATCATCCCAGATTACATC | |
| | GACATCAACCAGACAGTGCAGGACATCCTGGAGAACTACAG | |
| | GCCCAACTGGACAGTGCCAGAGTTCACCCTGGACATCTTCA | |
| | ACGCCACCTACCTGAACCTGACAGGAGAAATTGACGACCTG | |
| | GAGTTCAGATCAGAAAAACTTCACAACACCACCGTGGAGCT | |
| | TGCCATCCTCATTGACAACATTAACAACACACTGGTCAACCT | |
| | GGAATGGCTGAACAGAATTGAAACCTACGTGAAGTGGCCCT | |
| | GGTATGTGTGGCTGCTGATTGGACTGGTGGTGGTGTTCTGCA | |
| | TCCCACTGCTGCTGTTCTGCTGCTTCAGCACCGGCTGCTGTG | |
| | GATGCATCGGCTGCTTGGGCAGCTGCTGCCACAGCATCTGCA | |
| | GCAGGAGGCAGTTTGAGAACTACGAACCAATTGAAAAAGTG | |
| | CACGTCCAC | |
| 61 | TGTGAGCCAGTCATCACCTACTCCAACATCGGCGTGTGCAAG | SII (S3), |
| | AACGGAGCCCTGGTGTTCATCAACGTCACCCACTCAGACGG | amino acids |
| | CGACGTCCAGCCAATCTCCACAGGAAACGTCACCATCCCCAC | 782 to 1433 |
| | CAACTTCACCATCAGCGTGCAGGTGGAGTACATGCAGGTCTA | of S |
| | CACCACCCCAGTCTCCATCGACTGTGCCAGGTACGTGTGCAA | |
| | CGGCAACCCAAGATGCAACAAACTGCTGACCCAGTACGTGA | |
| | GCGCCTGCCAGACCATCGAGCAGGCCCTGGCCATGGGCGCC | |
| | AGGCTGGAGAACATGGAGGTGGACAGCATGCTCTTTGTGAG | |
| | CGAGAACGCCCTGAAGCTTGCCAGCGTGGAGGCCTTCAACA | |
| | GCACCGAAAACCTGGACTCCATCTACAAAGAGTGGCCCTCC | |
| | ATAGGAGGCTCCTGGCTGGGAGGCCTGAAGGACATCCTCCC | |
| | ATCCCACAACAGCAAAAGAAAGTACGGCAGCGCCATCGAAG | |
| | ACCTGCTGTTCGACAAGGTGGTCACCTCAGGACTGGGCACA | |
| | GTGGACGAGGACTACAAGAGGTGCACCGGAGGCTACGACAT | |
| | CGCAGACCTGGTCTGTGCCCAGTACTACAACGGCATCATGGT | |
| | GCTCCCAGGCGTGGCCAACGCCGACAAGATGACCATGTACA | |
| | CAGCAAGCCTGGCTGGAGGAATCACACTGGGAGCCCTGGGA | |
| | GGAGGGGCCGTGGCCATTCCATTCGCCGTGGCCGTGCAGGC | |
| | CAGACTGAACTACGTGGCCCTGCAGACAGACGTGCTAAACA | |
| | AGAACCAGCAGATCCTGGCCAACGCCTTCAACCAGGCCATC | |
| | GGCAACATCACCCAGGCCTTCGGCAAGGTGAACGACGCAAT | |
| | CCACCAGACATCACAGGGCCTGGCAACAGTGGCCAAGGCCC | |
| | TGGCCAAGGTCCAGGACGTGGTGAACACCCAGGGCCAGGCC | |
| | CTCTCACACCTGACAGTCCAGCTGCAGAACAACTTCCAGGC | |
| | AATCTCCTCCTCCATCTCAGACATCTACAACAGACTGGACCC | |
| | CCCCTCAGCCGACGCCCAGGTGGACAGACTCATCACAGGCA | |
| | GGCTGACCGCCCTCAACGCCTTCGTGTCCCAGACCCTCACCA | |
| | GGCAGGCCGAGGTGAGGGCCAGCAGGCAGCTCGCCAAGGA | |
| | CAAGGTGAACGAGTGCGTCAGAAGCCAGAGCCAGAGGTTC | |
| | GGCTTCTGTGGCAACGGCACCCACCTGTTCTCCCTGGCCAAC | |
| | GCAGCCCCCAACGGCATGATCTTCTTCCACACAGTCCTCCTC | |
| | CCAACAGCATATGAGACAGTCACCGCCTGGTCAGGAATCTGT | |
| | GCCTCAGACGGGGACAGAACCTTCGGCCTGGTGGTCAAGGA | |
| | CGTGCAGCTGACACTCTTCAGAAACCTGGACGACAAATTCTA | |
| | CCTGACCCCCAGGACCATGTACCAGCCAAGGGTGGCCACCT | |
| | CCTCAGACTTCGTGCAGATCGAGGGCTGTGACGTGCTCTTCG | |
| | TGAACGCCACCGTCATCGACCTCCCATCCATCATCCCAGACT | |
| | ACATCGACATCAACCAGACAGTGCAGGACATCCTGGAGAAC | |
| | TACCGCCCCAACTGGACCGTGCCAGAGTTCACCCTAGACATA | |
| | TTCAACGCCACCTACCTGAACCTGACAGGAGAAATTGACGA | |
| | CCTGGAGTTCAGATCAGAAAAGCTACACAACACCACCGTGG | |
| | AGTTAGCCATCCTCATAGACAACATTAACAACACCCTCGTCA | |
| | ACCTGGAGTGGCTCAACAGGATTGAAACCTACGTGAAGTGG | |
| | CCCTGGTACGTCTGGCTCCTCATCGGCCTGGTGGTGGTCTTCT | |
| | GCATCCCACTGCTGCTGTTCTGCTGCTTCTCCACCGGCTGCT | |
| | GTGGATGCATCGGCTGCCTGGGCTCATGCTGCCACTCAATCT | |
| | GCTCAAGGAGGCAGTTTGAAAACTACGAGCCAATAGAAAAA | |
| | GTCCACGTCCAC | |
| 62 | TGCGAGCCTGTTATTACCTACAGCAACATCGGTGTTTGTAAG | SII (S6), |
| | AATGGAGCTCTAGTCTTCATAAACGTAACGCACTCTGATGGC | amino acids |
| | GATGTTCAACCAATTTCCACTGGGAACGTAACCATACCCACC | 782 to 1433 |
| | AACTTTACTATTTCCGTCCAGGTGGAGTACATGCAAGTATATA | of S |
| | CCACGCCAGTGTCCATCGACTGCGCTCGGTATGTGTGCAACG | |
| | GTAACCCACGCTGCAATAAGCTGCTAACGCAGTACGTCAGCG | |
| | CCTGCCAGACAATAGAGCAGGCATTGGCAATGGGTGCAAGG | |
| | CTTGAAAACATGGAGGTGGACTCCATGTTGTTCGTGTCTGAA | |
| | AACGCTCTTAAACTAGCATCCGTGGAGGCATTCAACAGTACT | |
| | GAGAACTTGGACTCTATCTATAAGGAGTGGCCCTCCATTGGG | |
| | GGCAGCTGGCTTGGAGGTCTAAAAGACATCCTGCCCAGCCA | |
| | CAACTCCAAGAGGAAGTACGGGTCCGCTATAGAGGACCTCC | |
| | TCTTTGACAAGGTTGTTACTTCTGGTCTTGGCACAGTGGACG | |
| | AAGACTACAAGAGGTGCACAGGAGGCTATGATATAGCTGACC | |
| | TGGTGTGTGCTCAATACTACAACGGTATAATGGTTCTCCCAGG | |
| | TGTGGCCAACGCTGACAAGATGACAATGTACACAGCCTCTTT | |
| | AGCTGGAGGCATTACCCTGGGAGCCCTTGGGGGTGGCGCAG | |
| | TGGCAATTCCATTTGCCGTTGCGGTGCAGGCCCGACTAAACT | |
| | ATGTCGCACTTCAAACAGATGTGCTCAACAAGAACCAACAA | |
| | ATACTGGCCAACGCTTTCAACCAGGCCATTGGTAACATTACG | |
| | CAGGCATTTGGCAAGGTGAATGACGCCATCCACCAGACCAG | |
| | CCAGGGACTTGCCACAGTGGCCAAGGCCTTGGCAAAGGTGC | |
| | AGGATGTCGTGAACACACAGGGTCAGGCCCTCTCTCATTTGA | |
| | CAGTGCAGCTTCAGAATAACTTCCAAGCAATCAGTTCAAGCA | |
| | TCAGCGACATCTACAACCGGCTGGACCCCCCATCTGCAGATG | |
| | CGCAGGTGGACAGGCTAATCACTGGACGCTTGACGGCACTA | |
| | AATGCCTTTGTCAGCCAAACTCTGACCCGGCAAGCAGAGGT | |
| | GCGGGCCAGTAGACAACTGGCCAAAGACAAGGTCAACGAGT | |
| | GCGTCAGGTCCCAGTCCCAGCGTTTTGGATTCTGTGGGAACG | |
| | GGACGCACCTGTTCTCATTAGCCAATGCTGCACCCAATGGCA | |
| | TGATCTTTTTCCATACTGTTCTACTTCCTACTGCCTATGAAACC | |
| | GTGACCGCTTGGAGCGGCATCTGCGCATCTGATGGCGATAGG | |
| | ACCTTCGGGCTGGTCGTTAAGGATGTCCAGCTAACGCTGTTC | |
| | CGGAACTTGGATGACAAGTTCTACCTGACCCCCAGGACCATG | |
| | TACCAGCCGAGAGTGGCAACGAGTTCTGACTTCGTGCAAATT | |
| | GAGGGCTGTGACGTCCTGTTTGTTAATGCAACAGTGATCGAT | |
| | CTGCCCAGTATCATACCAGATTACATAGACATAAACCAGACA | |
| | GTCCAGGACATACTGGAGAATTACAGGCCAAACTGGACCGT | |
| | ACCAGAGTTCACGCTGGACATATTCAACGCTACGTACCTCAA | |
| | TTTGACTGGGGAAATTGATGACTTGGAGTTCAGGTCGGAGA | |
| | AGCTCCACAACACCACTGTGGAGCTGGCCATCCTGATTGACA | |
| | ACATCAACAACACTCTGGTGAACCTGGAGTGGCTAAATCGCA | |
| | TTGAAACCTATGTCAAGTGGCCTTGGTACGTTTGGCTACTGAT | |
| | CGGACTCGTGGTAGTCTTCTGCATACCACTCCTGCTATTTTGC | |
| | TGCTTCAGCACAGGGTGCTGTGGCTGCATTGGATGCCTAGGT | |
| | TCCTGCTGTCACAGTATCTGCAGCAGAAGACAATTCGAGAAC | |
| | TACGAGCCCATAGAAAAGGTCCACGTACAT | |
| 63 | GAGAGATACTGTGCCATGAAGGACGACAGCAGCAACACC | M, HF1902 |
| | TGCATCAACGGCACCAACAGCAGCTGCCAGACCTGCTTTG | strain |
| | AGAGAGGGGACCTGATCTGGCACCTGGCCAACTGGAACT | |
| | TCAGCTGGAGCGTGATCCTGATCGTGTTCATCACCGTGCT | |
| | GCAGTATGGAAGACCCCAGTTCAGCTGGCTGGTGTACGGC | |
| | ATCAAGATGCTGATCATGTGGCTGCTGTGGCCCATCGTGCT | |
| | GGCCCTGACCATCTTCAACGCCTACAGCGAGTACCAGGTG | |
| | TCCAGATACGTGATGTTTGGCTTCAGCATCGCCGGGGCCG | |
| | TGGTGACCTTCGCCCTGTGCATGATGTACTTCGTGAGGTC | |
| | CATCCAGCTGTACAGGAGGACAAAGTCATGGTGGTCCTTC | |
| | AACCCAGAAACCAATGCCATCCTGTGCGTCAACGCACTGG | |
| | GCAGAAGCTACGTCCTACCACTGGACGGCACTCCTACAGG | |
| | AGTGACCCTGACCCTGCTGTCAGGCAATCTGTACGCAGAG | |
| | GGGTTCAAGATGGCCGGTGGCCTGACCATCGAGCATCTGC | |
| | CTAAGTACGTGATGATCGCCACCCCTAGCAGGACAATCGT | |
| | GTACACCCTGGTGGGAAAGCAGCTAAAGGCGACCACAGC | |
| | CACAGGCTGGGCCTACTACGTGAAGTCCAAGGCAGGGGA | |
| | CTATTCAACCGAGGCCAGGACCGACAACCTGTCAGAGCA | |
| | CGAGAAGCTGCTGCACATGGTC | |
| 64 | GCTACACAAGGACAGAGAGTTAACTGGGGAGATGAACCAAG | N, HF1902 |
| | CAAGAGAAGAGACAGAAGCAACAGCAGAGGAAGAAAAAAT | strain |
| | GGAAACATCCCCCTGTCCTACTTCAACCCCATCACCCTGGAG | |
| | AGCGGCAGCAAGTTCTGGAACATCTGCCCCAGGGACTTTGT | |
| | GCCCAAGGGCATTGGAAACAAGGACCAGCAGATCGGCTACT | |
| | GGAACAGACAGGTGCGCTACAGAATTGTGCGGGGCCAGAGG | |
| | AAGGAGCTGCCCGAGAGATGGTTCTTCTACTTCTCTGGAACA | |
| | GGCCCCCACGCTGATGCCAAGTTCAAGGACAAGATTGATGG | |
| | AGTGTTCTGGGTGGCCAGAGATGGAGCCATGAACAAGCCCA | |
| | CCACCCTGGGCACCAGAGGCACCAACAACGAGAGCAAGCC | |
| | CCTGAAGTTTGATGGCAAGATCCCCCCCCAGTTCCAGCTGGA | |
| | GGTGAACAGAAGCAGAAACAACAGCAGAAGCGGCAGCCAG | |
| | CCCAGGAGCGTGTCCAGAAGCAGAAGCCAGAGCAGAGGAA | |
| | GACAGCAGAGCAACAACCAGAACACCAACGTGGAGGACAC | |
| | CATCGTGGCCGTGCTGAGCAAGCTGGGCGTGACAGACAAGC | |
| | AGAGGAGCAGAAGCAAGTCTGGAGAAAGAAACCAGAGCAA | |
| | GCCCAGAGACACCACCCCCAAGAATGCCAACAAGCACACCT | |
| | GGAAGAAGACAGCTGGCAAGGGGGACGTGACCAACTTCTAT | |
| | GGAGCCAGGAGCAGCAGCGCCAACTTTGGAGACAGCGACCT | |
| | GGTGGCCAATGGAAATGCCGCCAAGTGCTACCCCCAGATCGC | |
| | CGAGTGTGTGCCCAGCGCCAGCAGCATCCTGTTTGGCAGCC | |
| | AGTGGAGCGCCGAGGAGGCCGGGGACCAGGTGAAGGTGAC | |
| | CCTGACCCACACCTACTACCTGCCCAAGGATGATGCCAAGAC | |
| | CAGCCAGTTCCTGGAGCAGATTGATGCCTACAAGAGGCCCA | |
| | GCGAGGTGGCCAAGGACCAGAGGCAGAGGAAGAGCAGAAG | |
| | CAAGTCTGCTGACAAGAAGCCAGAGGAGCTGTCGGTGACCC | |
| | TCGTCGAGGCATACACCGATGTCTTCGACGACACTCAGGTGG | |
| | AGATGATCGACGAGGTCACCAAC | |
| 65 | TGCAGCAGCGTGATCACCTACAGCAGCTTCGCCATCTGCAAC | SII, HF1902 |
| | ACAGGAGAGATCAAGTACGTGAACGTGACCCACGTGGAGAC | strain |
| | TGTGGACGACAACATCGGGGTGATCAAGCCCATCAGCACCG | |
| | GCAACATCACCATCCCCAAGAACTTCACAGTGGCCGTGCAG | |
| | GCCGAGTACATCCAGATCCAGGTGAAGCCTGTGGTGGTGGA | |
| | CTGCGCCAAGTACGTGTGCAATGGAAATGGCCACTGCCTGAA | |
| | CCTGCTGACCCAGTACACCTCTGCCTGCCAGACCATCGAGAA | |
| | CGCCCTGAACCTGGGCGCCAGACTGGAGTCCCTGATGCTGTC | |
| | TGAGATGGTGACCGTGAGCGAGAGAAACCTGGACCTGGCCA | |
| | CCGTGGAGAAGTTCAACAGCACAGTGCTGGGCGGGGAGAA | |
| | GCTGGGAGGCTTCTACTTTGACGGCCTGAAGAGCCTGCTGCC | |
| | TCCCACCATCGGCAAGAGAAGCGCCGTGGAGGACCTGCTGT | |
| | TCAACAAGGTGGTGACCAGCGGCCTGGGGACCGTGGATGAT | |
| | GACTACAAGAAGTGCAGCGCCGGCACAGATGTGGCCGACCT | |
| | GGCCTGTGCCCAGTACTACAACGGCATCATGGTGCTGCCTGG | |
| | AGTGGTGGACCAGAACAAGATGGCCATGTACACCGCCAGCC | |
| | TGATTGGAGGCATGGCCCTGGGCAGCATCACCAGCGCCGTG | |
| | GCCGTGCCCTTCGCCATGCAGGTGCAGGCCAGACTGAACTAC | |
| | GTGGCCCTGCAGACAGATGTGCTGCAGGAGAACCAGAAGAT | |
| | CCTGGCCAACGCCTTCAACAACGCCATCGGCAACATCACCCT | |
| | GGCCCTGGGGAAGGTGAGCAACAGCATCACCACCATCTCTG | |
| | GAGGCTTCCACACCATGGCCAGCGCCCTGACCAAGATCCAG | |
| | AGCGTGGTGAACCAGCAGGGGGAGGCCCTGTCCCAGCTGAC | |
| | CAGCCAGCTGCAGAAGAACTTCCAGGCCATCTCCTCTTCCAT | |
| | TGCCGAGATCTACAACAGACTGGAGAAGGCTGAGGCCGACG | |
| | CCCAGGTGGACAGGCTGATCACAGGAAGACTGGCCGCCCTG | |
| | AACGCCTACGTGTCCCAGACCCTGACCCAGTATGCCGAGGTG | |
| | AAGGCCAGCAGACAGCTGGCCATGGAGAAGGTGAATGAGTG | |
| | TGTGAAGAGCCAGTCTGACAGATACGGCTTCTGTGGAAATGG | |
| | AACCCACCTGTTCAGCCTGGTGAACTCTGCCCCTGACGGCCT | |
| | GCTGTTCTTCCACACCGTGCTGCTGCCCACAGAGTGGGAGG | |
| | AGGTGACAGCCTGGAGTGGCATCTGTGTGAATGACACCTACG | |
| | CCTACGTGCTGAAAGACTTTGACTACAGCATCTTCAGCTACA | |
| | ACGGCACCTACATGGTGACCCCCAGAAACATGTTCCAGCCCA | |
| | GAAAGCCCCAGATGTCAGACTTCGTGCAGATCACCAGATGC | |
| | GAGGTGACCTTCCTGAACACAACCTACACCACCTTCCAGGA | |
| | GATCGTGATCGACTACATCGACATCAACAAGACCATCGCCGA | |
| | CATGCTGGAGCAGTACAACCTGAACTACACAACCCCTGAGCT | |
| | GAACCTGCAGCTGGAGATCTTCAACCAGACCAAGCTGAACC | |
| | TGACCGCCGAGATCGACCAGCTGGAGCAGAGAGCCGACAAC | |
| | CTGACCAACATCGCCCACGAGCTGCAGCAGTACATCGACAA | |
| | CCTGAACAAGACCCTGGTGGACCTGGAGTGGCTGAACAGAA | |
| | TTGAAACCTACGTGAAGTGGCCCTGGTACGTGTGGCTGCTGA | |
| | TCGGCCTGGTGATCGTGTTCTGCATCCCTCTGCTGCTGTTCTG | |
| | CTGCCTGAGCACCGGCTGCTGTGGCTGCTTCGGCTTCCTGGG | |
| | CTCCTGCTGCCACTCCCTGTGCAGCAGGAGGCAGTTTGAGTC | |
| | CTACGAGCCCATCGAGAAGGTGCACATCCAC | |
| 66 | GAGAGATACTGTGCCATGCAGGACAGCGGCCTGCAGTGCATC | M, SH2211 |
| | AACGGCACCAACAGCAGATGCCAGACCTGCTTTGAAAGAGG | strain |
| | GGACCTGATCTGGCACCTGGCCAACTGGAACTTCTCCTGGAG | |
| | CGTGATCCTGATCGTGTTCATCACCGTGCTGCAGTACGGCAG | |
| | ACCACAGTTCTCATGGCTTGTCTATGGCATCAAGATGCTGATT | |
| | ATGTGGCTGCTTTGGCCTATCGTCCTGGCCCTGACCATCTTCA | |
| | ACGCCTACTCTGAGTACCAGGTGTCAAGGTATGTCATGTTCG | |
| | GCTTCTCAGTGGCTGGAGCTGTGGTGACCTTTGCTCTGTGGA | |
| | TGATGTACTTCGTGAGGTCCGTGCAGCTGTACAGGAGGACAA | |
| | AGTCATGGTGGTCCTTCAACCCAGAAACCAATGCCATCCTGT | |
| | GCGTCAACGCACTGGGCAGAAGCTACGTCCTACCACTGGAC | |
| | GGCACTCCTACAGGAGTGACCCTGACCCTGCTGTCAGGCAAT | |
| | CTGTACGCAGAGGGGTTCAAGATGGCCGGTGGCCTGACCATC | |
| | GAGCATCTGCCTAAGTACGTGATGATCGCCACCCCTAGCAGG | |
| | ACAATCGTGTACACCCTGGTGGGAAAGCAGCTAAAGGCGAC | |
| | CACAGCCACAGGCTGGGCCTACTACGTGAAGTCCAAGGCAG | |
| | GGGACTATTCAACCGAGGCCAGGACAGACAACCTGAGCGAG | |
| | CACGAGAAGCTGCTGCACATGGTG | |
| 67 | GCTACACAAGGACAGAGAGTTAACTGGGGAGATGAACCAAG | N, SH2211 |
| | CAAGAGAAGAGGAAGAAGCAACAGCAGAGGAAGAAAGAA | strain |
| | CAATGACATCCCCCTGTCCTTCTACAACCCCATCACCCTGGA | |
| | GCAGGGCAGCAAGTTCTGGAACCTGTGCCCCAGGGACCTGG | |
| | TGCCCAAGGGCATCGGCAACAAGGACCAGCAGATTGGCTAC | |
| | TGGAACAGACAGATCAGATACAGAATTGTGAAGGGCCAGAG | |
| | GAAGGAGCTGGCCGAGAGGTGGTTCTTCTACTTCCTGGGCA | |
| | CCGGCCCCCATGCTGATGCCAAGTTCAAGGACAAGATTGATG | |
| | GAGTGTTCTGGGTGGCCAGAGATGGGGCCATGAACAAGCCC | |
| | ACCACCCTGGGCACCAGGGGCACCAACAATGAGAGCAAGCC | |
| | CCTGAGGTTTGATGGCAAGATCCCCCCCCAGTTCCAGCTGGA | |
| | GGTGAACAGAAGCAGAAACAACAGCAGAAGCGGCAGCCAG | |
| | AGCAGAAGTGTGTCCAGAAACAGAAGCCAGAGCAGAGGAA | |
| | GACACCACAGCAACAACCAGAACAACAATGTGGAGGACAC | |
| | CATCGTGGCCGTGCTGGAGAAGCTGGGGGTGACAGACAAGC | |
| | AGAGGAGCAGAAGCAAGCCCAGAGAAAGAAGTGACAGCAA | |
| | GCCCAGGGACACCACCCCCAAGAATGCCAACAAGCACACCT | |
| | GGAAGAAGACAGCTGGAAAAGGAGATGTGACCACCTTCTAT | |
| | GGAGCCAGGAGCAGCAGCGCCAACTTTGGAGACAGTGACCT | |
| | GGTGGCCAATGGAAATGCTGCCAAGTGCTACCCCCAGATTGC | |
| | TGAGTGTGTGCCCTCTGTGAGCAGCATCATCTTTGGCAGCCA | |
| | GTGGTCAGCAGAGGAGGCTGGGGACCAGGTGAAGGTGACC | |
| | CTGACCCACACCTACTACCTGCCCAAGGATGATGCCAAGACC | |
| | AGCCAGTTCCTGGAGCAGATTGATGCCTACAAGAGGCCCAG | |
| | CGAGGTGGCCAAGGACCAGAGGCAGAGGAGGAGCCTGAGC | |
| | AAGTCTGCTGACAAGAAGCCAGAGGAGCTGTCGGTGACCCT | |
| | CGTCGAGGCGTACACCGACGTCTTCGACGACACTCAGGTGG | |
| | AGATGATCGACGAGGTCACCAAC | |
| 68 | TGTGAGCCAGTCATCACCTACAGCAACATCGGAGTGTGCAAG | SII, SH2211 |
| | AACGGAGCCCTGGTGTTCATCAACGTGACCCACAGCGACGG | strain |
| | AGATGTCCAGCCCATCAGCACAGGAAATGTGACCATCCCAAC | |
| | CAACTTCACCATCAGCGTCCAGGTGGAATACATGCAGGTGTA | |
| | CACCACCCCAGTGTCCATCGACTGTGCCAGATACGTGTGCAA | |
| | TGGAAACCCCAGATGCAACAAGCTCCTCACCCAGTACGTGTC | |
| | AGCCTGCCAGACAATCGAGCAGGCCCTGGCCATGGGAGCCA | |
| | GGCTCGAGAACATGGAAGTGGACAGCATGCTGTTTGTCTCA | |
| | GAGAATGCCCTGAAACTGGCCAGCGTGGAGGCCTTCAACAG | |
| | CACAGAGAACCTGGACCCCATCTACAAGGAGTGGCCATCAAT | |
| | CGGAGGCAGCTGGCTGGGAGGACTTAAGGACATCCTGCCAA | |
| | GCCACAACAGCAAAAGAAAGTACGGCAGCGCCATTGAGGAC | |
| | CTGCTGTTTGACAAGGTGGTCACCTCCGGCCTGGGCACAGT | |
| | GGATGAGGACTACAAGAGATGCACCGGCGGCTATGACATTGC | |
| | CGACCTGGTGTGTGCCCAGTACTACAATGGCATCATGGTGCT | |
| | GCCTGGAGTGGCCAACGCCGACAAAATGACCATGTACACCG | |
| | CCTCCCTGGCTGGAGGCATCACACTGGGAGCCCTGGGGGGA | |
| | GGAGCAGTGGCCATCCCCTTTGCAGTGGCTGTGCAGGCCAG | |
| | ACTCAACTACGTGGCCCTGCAGACAGACGTGCTCAACAAGA | |
| | ACCAGCAGAACCTGGCCAATGCCTTCATCCAGGCTATCGGAA | |
| | ACATCACCCAGGCCTTTGGAAAAGTGAATGATGCCATCCACC | |
| | AGACCAGCCAGGGCCTGGCCACAGTGGCCAAGGCCCTGGCC | |
| | AAGGTGCAGGACGTGGTCAACACCCAGGGCCAGGCCCTCAG | |
| | TCACCTCACAGTACAGCTCCAGAACAACTTCCAGGCAATCTC | |
| | CTCCTCCATCAGCGACATCTACAACAGGCTGGACCCCCCAAG | |
| | CGCTGATGCCCAGGTGGACAGACTGATCACAGGAAGACTCA | |
| | CAGCCCTCAACGCATTTGTGTCCCAGACACTGACCAGGCAG | |
| | GCAGAGGTCAGGGCCAGCAGGCAGCTGGCCAAGGACAAGG | |
| | TGAATGAGTGCGTGAGGAGCCAGAGCCAGAGATTTGGCTTC | |
| | TGCGGAAACGGCACCCACCTGTTCAGCCTGGCCAACGCCGC | |
| | CCCCAACGGCATGATTTTCTTCCACACAGTCCTCCTCCCCAC | |
| | AGCCTACGAAACAGTGACAGCCTGGTCAGGCATCTGTGCCA | |
| | GCGACGGAGACAGAACCTTTGGCCTGGTGGTGAAGGATGTG | |
| | CAGCTCACCCTCTTCAGAAACCTGGATGACAAGTTCTACCTC | |
| | ACCCCAAGAACCATGTACCAGCCCAGAGTGGCCACAAGCAG | |
| | CGACTTTGTGCAGATTGAGGGCTGTGACGTGCTGTTTGTGAA | |
| | TGCAACAGTGATTGACCTCCCAAGCATCATCCCAGATTACATC | |
| | GACATCAACCAGACAGTGCAGGACATCCTGGAGAACTACAG | |
| | GCCCAACTGGACAGTGCCAGAGTTCACCCTGGACATCTTCA | |
| | ACGCCACCTACCTGAACCTGACAGGAGAAATTGACGACCTG | |
| | GAGTTCAGATCAGAAAAACTTCACAACACCACCGTGGAGCT | |
| | TGCCATCCTCATTGACACCATTAACAACACACTGGTCAACCT | |
| | GGAATGGCTGAACAGAATTGAAACCTACGTGAAGTGGCCCT | |
| | GGTATGTGTGGCTGCTGATTGGACTGGTGGTGGTGTTCTGCA | |
| | TCCCACTGCTGCTGTTCTGCTGCTTCAGCACCGGCTGCTGTG | |
| | GATGCATCGGCTGCTTGGGCAGCTGCTGCCACAGCATCTGCA | |
| | GCAGGAGGCAGTTTGAGTACTACGAGCCCATCGAGAAGGTG | |
| | CACGTGCAC | |
| 69 | GACCGCTACTGTGCCATGCAGCACGCCAGCAGCACCAGCTG | M, 2-C11 Re |
| | CATCAATGGCACCAGCACCAACAGCTGCCAGACCTGCTTTGA | 10276 strain |
| | AAGAGGAGACTTGATTTGGCACCTGGCCAACTGGAACTTCA | |
| | GCTGGAGCGTCATCCTCATCGTGTTCATCACCGTGCTGCAGT | |
| | ATGGAAGACCCCAGCTGAGCTGGTTTGTGTACGGCATCAAGA | |
| | TGCTCATCATGTGGCTGCTGTGGCCCATCGTGCTGGCCCTGA | |
| | CCATCTTCAACGCCTACAGCGAGTACCAGGTGTCCAGATACG | |
| | TGATGTTTGGCTTCTCTGTGGCTGGAGCTGTCATCACCTTTGC | |
| | CCTGTGGATGATGTACTTTGTGAGGAGCATCCAGCTGTACAG | |
| | GAGGACCAAGAGCTGGTGGAGCTTCAACCCAGAAACCAATG | |
| | CCATCCTGTGTGTGAATGCCCTGGGCAGGAGCTACGTGCTGC | |
| | CCCTGGATGGCATCCCCACTGGAGTCACCCTGACCCTGCTGT | |
| | CTGGAAACCTGTATGCTGAGTGCTTCAAGATGGTGGGCGGCC | |
| | TGACCATCGAGCACCTGCCCAAGTACGTGATGATTGCCACCC | |
| | CCAGCAGCACCATCGTGTACACCCTGGTGGGCAAGCAGCTG | |
| | AAGGCCACCACAGCCACCGGCTGGGCCTACTATGTGAAGAG | |
| | CAAGGCTGGAGACTACAGCACAGAGGCCAGGACAGACAAC | |
| | CTGTCTGAACATGAGAAGCTGCTGCACATGGTG | |
| 70 | GCCACCCAGGGCCAGAGGGTCAACTGGGGCGACGAGCCCA | N, 2-C11 Re |
| | GCAAGAGGAGAGGAAGAAGCAACAGCAGAGGAAGGAAGA | 10276 strain |
| | ACAATGACATCCCCCTGAGCTTCTACAACCCCATCACCCTGG | |
| | AGACTGGCAGCAAGTTCTGGAATGTCTGCCCCAGGGACTTT | |
| | GTGCCCAAGGGCATCGGCAACAAGGACCAGCAGATTGGCTA | |
| | CTGGAACAAGCAGGCCCGCTACAGAATTGTGAAGGGCCAGA | |
| | GGAAGGACCTGCCTGAGAGGTGGTTCTTCTACTTCCTGGGCA | |
| | CAGGCCCCCACGCTGATGCCAAGTTCAAGGACAAGATTGAT | |
| | GGAGTCTTCTGGGTGGCCAAGGATGGAGCCATGAACAAGCC | |
| | CACCACCCTGGGCACCAGAGGCACCAACAATGAGAGCAAGC | |
| | CCCTGAGATTTGATGGGAAGATCCCCCCCCAGTTCCAGCTGG | |
| | AGGTGAACCAGAGCAGAAACAACAGCAGAAGCGGCAGCCA | |
| | GAGCAGAAGTGCCTCCAGAAACAGAAGCCAGAGCAGAGGA | |
| | AGACAGCAGAGCAACAACCAGAACACCAACGTGGAGGACA | |
| | CCATCGTGGCTGTGCTGCAGAAGCTGGGCGTGACAGACAAG | |
| | CAGAGGAGCAGAAGCAAGAGCAGAGAAAGAAGCGGCAGCA | |
| | ACAGCAGGGACACCACCCCCAAGAATGCCAACAAGCACAGC | |
| | TGGAAGAAGACAGCTGGCAAGGGAGATGTGACCAACTTCTA | |
| | TGGAGCCAGGAGTGCCAGCGCCAACTTCGGGGACAGTGACC | |
| | TGGTGGCCAATGGAAATGCCGCCAAGTGCTACCCCCAGATTG | |
| | CTGAGTGTGTGCCCAGCGTGTCCTCCATGCTGTTTGGAAGCC | |
| | AGTGGTCAGCAGAGGATGCTGGGGACCAGGTGAAGGTGACC | |
| | CTGACCCACACCTACTACCTGCCCAAGGATGATGCCAAGACC | |
| | AGCCAGTTCCTGGGCCAGATTGATGCCTACAAGAGGCCCAGC | |
| | CAGGTGGTGAAGGAGCAGAGGCAGAGGAAGAGCAGAAGCA | |
| | AGTCTGCTGACAAGAAGCCAGAGGAGCTGTCTGTGACCCTG | |
| | GTGGAGGCCTACACAGACGTGTTTGATGACACCCAGGTGGA | |
| | GATGATTGATGAGGTGACCAAC | |
| 71 | TGTGAGCCCATCATCACCTACTTCAACATCGGGGTGTGCAAG | SII, 2-C11 |
| | AATGGGGCCCTGGTCTTCATCAATGTGACCCACAGCGATGGA | Re 10276 |
| | GATGTGCAGCCCATCAGCACAGGAAATGTGACCATCCCCACC | strain |
| | AACTTCACCATCTCTGTGCAGGTGGAGTACATCCAGGTGTAC | |
| | ACCACCCCTGTGTCCATCGACTGCAGCCGCTACGTGTGCAAT | |
| | GGAAACCCCAGGTGCAACAAGCTGCTGACCCAGTACTTCTC | |
| | TGCCTGCCAGACCATCGAGCAGGCCCTGGCCATGGGAGCCC | |
| | GGCTGGAGAACATGGAGGTGGACAGCATGCTGTTTGTGTCT | |
| | GAAAATGCCCTGAAGCTGGCCTCTGTGGAGGCCTTCAACAG | |
| | CAGTGAGCACCTGGACCCCATCTACAAGGAGTGGCCCAACA | |
| | TTGGAGGCAGCTGGCTGGGGGGCCTGAAGGACATCCTGCCC | |
| | AGCCACAACAGCAAGAGGAACTACAGAAGTGCCATCGAGGA | |
| | CCTGCTCTTTGACAAGGTGGTGACCTCTGGGCTGGGCACCGT | |
| | GGATGATGACTACAAGAGGTGCACAGGAGGCTATGACATTGC | |
| | AGACCTGGTGTGTGCCCAGTACTACCATGGCATCATGGTGCT | |
| | GCCTGGAGTGGCCAATGATGACAAGATGACCATGTACACAGC | |
| | CTCCCTGGCTGGAGGCATCACCCTGGGGGCCCTGGGCGGAG | |
| | GAGCCGTGGCCATCCCCTTTGCTGTGGCTGTGCAGGCCAGGC | |
| | TCAACTACGTGGCCCTGCAGACAGATGTGCTCAACAAGAAC | |
| | CAGCAGATCCTGGCCAACGCCTTCAACCAGGCCATTGGAAA | |
| | CATCACCCAGGCCTTTGGGAAGGTGAATGATGCCATCCACCA | |
| | GACCAGCAAGGGCCTGGCCACCGTGGCCAAGGCCCTGGCCA | |
| | AGGTGCAGGATGTGGTCAACACCCAGGGCCAGGCCCTGAGC | |
| | CACCTCACTGTCCAGCTGCAGAACAACTTCCAGGCCATCAGC | |
| | AGCAGCATCTCTGACATCTACAACAGGCTGGATGAGCTGTCT | |
| | GCTGATGCCCAGGTGGACAGACTCATCACCGGGAGGCTGAC | |
| | AGCCCTGAATGCCTTTGTCAGCCAGACCCTGACCAGGCAGG | |
| | CAGAGGTGCGGGCCTCCCGGCAGCTGGCCAAGGACAAGGTG | |
| | AATGAGTGTGTGAGGAGCCAGAGCCAGAGGTTTGGCTTCTG | |
| | TGGAAATGGCACCCACCTCTTCTCCCTGGCCAATGCTGCCCC | |
| | CAATGGCATGATCTTCTTCCACACCGTGCTGCTGCCCACCGC | |
| | CTATGAAACAGTGACAGCCTGGAGCGGCATCTGTGCCTCTGA | |
| | TGGGGACCACACCTTCGGCCTGGTGGTGAAGGATGTCCAGC | |
| | TGACCCTCTTCAGAAACCTGGATGACAAGTTCTACCTGACCC | |
| | CCAGGACCATGTACCAGCCCCGGGTGGCCACCAGCAGCGAC | |
| | TTTGTGCAGATTGAGGGCTGTGATGCCCTGTTTGTGAATGCC | |
| | ACTGTCATCGAGCTGCCCAGCATCATCCCAGACTACATTGAC | |
| | ATCAACCAGACCGTGCAGGACATCCTGAAGAACTACAGGCC | |
| | CAACTGGACAGTTCCTGAGCTGACCCTGGACATCTTCAACAG | |
| | CACCTACCTGAACCTGACAGGAGAAATCAATGACCTGGAGTT | |
| | CAGAAGTGAGAAGCTGCACAACACCACAGTGGAGCTGGCTG | |
| | TGCTGATCGACAACATCAACAACACCCTGGTCAACCTGGAGT | |
| | GGCTGAACAGAATTGAAACCTACGTGAAGTGGCCCTGGTATG | |
| | TTTGGCTGCTCATCGGCCTGGTGCTGGTGTTCTGCATCCCCCT | |
| | GCTCATGTTCTGCTGCCTGAGCACCGGCTGCTGCGGCTGCTT | |
| | CGGCTGCCTGGGCAGCTGCTGCCACAGCCTGTTCTCCAGAA | |
| | GACACTTTGAGAACTACGAGCCCATCGAGAAGGTGCACATC | |
| | CAC | |
| 72 | CTGGTCTTCCTGCATGCTGTGCTGGTGACTGTGCTCATCCTGC | 7a |
| | CCCTCATCGGCCGCATCCAGCTGCTGGAGAGACTTCTCCTGA | |
| | GCCACCTGCTGAACCTGACCACAGTCAGCAATGTCCTGGGG | |
| | GTCCCAGACAGCAGCCTGCGGGTCAACTGCCTGCAGCTGCT | |
| | GAAGCCAGACTGCCTGGACTTCAACATCCTGCACAAGGTGC | |
| | TGGCAGAAACACGGCTGCTGGTGGTGGTGCTGCGGGTCATC | |
| | TTCCTGGTGCTGCTGGGCTTCAGCTGCTACACCCTGCTGGGG | |
| | GCCCTCTTC | |
| 73 | GATGCTGTGAAGAGCATCGGCATCTCTGTGGATGCTGTGCTG | 3a |
| | GATGAGCTGGACAGCATTGCCTTTGCTGTCACCCTGAAGGTG | |
| | CTCTTCAACAGCGGGAAGCTGCTGGTGTGCATCGGCTTCGGG | |
| | GACACCTTTGAGGAGGCTGAGCAGAAGGCCTATGCCAAGAG | |
| | CAAGCTGGTG | |
| 74 | ATGTACCCCTACGATGTGCCAGACTACGCCTACCCCTACGATG | HA-M(M2) |
| | TGCCAGACTACGCCTACCCCTACGATGTGCCAGACTACGCCG | |
| | AGAGATACTGTGCCATGCAGAACACAGGCAGCCAGTGCATC | |
| | AATGGAACAGACAGCAGCTGCAGCACCTGCTTTGAAAGAGG | |
| | AGGCCTGATCTGGCACCTGGCCAACTGGAACTTCAGCTGGA | |
| | GTGTGATCCTGATTGTCTTCATTACAGTGCTGAAGTACGGCA | |
| | GGCCCCAGTTCAGCTGGCTGGTGTACGGCATCAAGATGCTCA | |
| | TCATGTGGCTGCTGTGGCCCATTGTGTTGGCCCTCACCATCTT | |
| | CAATGCCTACAGCGAGTACCAGGTGTCCAGATACGTGATGTT | |
| | CGGCTTCTCAGTGGCAGGAGCCGTGGTGACCTTTGCCCTCTG | |
| | GATGATGTACTTTGTGAGGTCCATCCAGCTCTACAGAAGAAC | |
| | AAAGAGCTGGTGGAGCTTCAACCCAGAAACCAACGCCATCC | |
| | TGTGTGTCAATGCCCTGGGCAGATCCTATGTGCTGCCCCTGG | |
| | ATGGCACCCCCACAGGCGTCACCCTCACCCTCCTGAGCGGC | |
| | AACCTGTACGCCGAGGGCTTCAAGATGGCCGGCGGCCTGAC | |
| | AATCGAGCACCTGCCCAAGTATGTGATGATTGCCACCCCCAG | |
| | CAGGACAATAGTCTACACCCTGGTGGGAAAACAGCTGAAGG | |
| | CTACCACAGCCACAGGCTGGGCCTACTACGTCAAGAGCAAG | |
| | GCCGGCGACTACAGCACAGAGGCCAGGACCGACAACCTGTC | |
| | CGAACATGAAAAACTCCTGCACATGGTC | |
| 75 | ATGTACCCCTACGACGTGCCCGACTACGCCTACCCCTACGAC | HA-M(M3) |
| | GTGCCAGACTACGCCTACCCCTACGACGTGCCAGACTACGCC | |
| | GAGAGGTACTGCGCCATGCAGAACACAGGCTCCCAGTGCAT | |
| | CAACGGAACAGACTCCTCCTGCAGCACCTGCTTCGAGAGAG | |
| | GAGGCCTCATTTGGCACCTGGCCAACTGGAACTTCAGCTGGT | |
| | CAGTCATTCTGATAGTCTTCATAACAGTGCTGAAGTACGGCA | |
| | GGCCCCAGTTCTCCTGGCTCGTGTATGGCATCAAGATGCTGAT | |
| | CATGTGGCTGCTGTGGCCCATCGTCCTGGCCCTGACCATCTTC | |
| | AACGCCTACTCAGAGTACCAGGTCAGCAGGTACGTGATGTTC | |
| | GGCTTCTCCGTGGCCGGAGCAGTGGTGACCTTCGCCCTGTGG | |
| | ATGATGTACTTCGTGAGGAGCATCCAACTGTACAGGAGGACC | |
| | AAAAGCTGGTGGTCCTTCAACCCAGAAACCAACGCCATCCT | |
| | CTGCGTGAACGCCCTGGGCAGGTCCTACGTCCTCCCCCTGGA | |
| | CGGCACCCCCACCGGGGTCACCCTCACCCTCCTGTCAGGGA | |
| | ACCTGTACGCTGAGGGCTTCAAGATGGCTGGAGGCCTGACA | |
| | ATTGAACACCTGCCCAAGTACGTCATGATCGCAACACCCTCC | |
| | AGAACCATCGTCTACACCCTGGTGGGCAAGCAGCTGAAGGC | |
| | CACCACCGCCACCGGCTGGGCCTACTACGTCAAGTCCAAGG | |
| | CCGGCGACTACAGCACCGAGGCCAGGACCGACAACCTCTCA | |
| | GAGCACGAGAAGCTGCTGCACATGGTG | |
| 76 | ATGTACCCATACGATGTTCCAGACTACGCTTACCCATATGACG | HA-M(M6) |
| | TGCCAGACTATGCCTACCCCTACGACGTGCCCGACTACGCAG | |
| | AGAGGTACTGTGCCATGCAGAACACCGGTTCCCAGTGCATCA | |
| | ACGGCACAGACTCCTCCTGCTCCACCTGCTTCGAGAGAGGA | |
| | GGCCTGATCTGGCACCTGGCAAACTGGAACTTCAGCTGGAG | |
| | CGTGATCCTGATAGTGTTCATAACCGTCCTGAAGTACGGCAG | |
| | ACCACAGTTCTCATGGCTTGTCTATGGCATCAAGATGCTGATT | |
| | ATGTGGCTGCTTTGGCCTATCGTCCTGGCCCTGACCATCTTCA | |
| | ACGCCTACTCTGAGTACCAGGTGTCAAGGTATGTCATGTTCG | |
| | GCTTCTCAGTGGCTGGAGCTGTGGTGACCTTTGCTCTGTGGA | |
| | TGATGTACTTCGTGAGGTCCATCCAGCTGTACAGGAGGACAA | |
| | AGTCATGGTGGTCCTTCAACCCAGAAACCAATGCCATCCTGT | |
| | GCGTCAACGCACTGGGCAGAAGCTACGTCCTACCACTGGAC | |
| | GGCACTCCTACAGGAGTGACCCTGACCCTGCTGTCAGGCAAT | |
| | CTGTACGCAGAGGGGTTCAAGATGGCCGGTGGCCTGACCATC | |
| | GAGCATCTGCCTAAGTACGTGATGATCGCCACCCCTAGCAGG | |
| | ACAATCGTGTACACCCTGGTGGGAAAGCAGCTAAAGGCGAC | |
| | CACAGCCACAGGCTGGGCCTACTACGTGAAGTCCAAGGCAG | |
| | GGGACTATTCAACCGAGGCCAGGACCGACAACCTGTCAGAG | |
| | CACGAGAAGCTGCTGCACATGGTC | |
| 77 | ATGTACCCCTACGATGTGCCAGACTACGCCTACCCCTACGATG | HA- |
| | TGCCAGACTACGCCTACCCCTACGATGTGCCAGACTACGCCG | M_d(M2) |
| | AGAGATACTGTGCCATGCAGAACACAGGCAGCCAGTGCATC | |
| | AATGGAACAGACAGCAGCTGCAGCACCTGCTTTGAAAGAGG | |
| | AGGCCTGATCTGGCACCTGGCCAACTGGAACTTCAGCTGGA | |
| | GTGTGATCCTGATTGTCTTCATTACAGTGCTGAAGTACGGCA | |
| | GGCCCCAGTTCAGCTGGCTGGTGTACGGCATCATTGTGTTGG | |
| | CCCTCACCATCTTCAATGCCTACAGCGAGTACCAGGTGTCCA | |
| | GATACGTGATGTTCGGCTTCTCAGTGGCAGGAGCCGTGGTGA | |
| | CCTTTGCCCTCTGGATGATGTACTTTGTGAGGTCCATCCAGCT | |
| | CTACAGAAGAACAAAGAGCTGGTGGAGCTTCAACCCAGAAA | |
| | CCAACGCCATCCTGTGTGTCAATGCCCTGGGCAGATCCTATGT | |
| | GCTGCCCCTGGATGGCACCCCCACAGGCGTCACCCTCACCCT | |
| | CCTGAGCGGCAACCTGTACGCCGAGGGCTTCAAGATGGCCG | |
| | GCGGCCTGACAATCGAGCACCTGCCCAAGTATGTGATGATTG | |
| | CCACCCCCAGCAGGACAATAGTCTACACCCTGGTGGGAAAA | |
| | CAGCTGAAGGCTACCACAGCCACAGGCTGGGCCTACTACGT | |
| | CAAGAGCAAGGCCGGCGACTACAGCACAGAGGCCAGGACC | |
| | GACAACCTGTCCGAACATGAAAAACTCCTGCACATGGTC | |
| 78 | ATGTACCCCTACGACGTGCCCGACTACGCCTACCCCTACGAC | HA- |
| | GTGCCAGACTACGCCTACCCCTACGACGTGCCAGACTACGCC | M_d(M3) |
| | GAGAGGTACTGCGCCATGCAGAACACAGGCTCCCAGTGCAT | |
| | CAACGGAACAGACTCCTCCTGCAGCACCTGCTTCGAGAGAG | |
| | GAGGCCTCATTTGGCACCTGGCCAACTGGAACTTCAGCTGGT | |
| | CAGTCATTCTGATAGTCTTCATAACAGTGCTGAAGTACGGCA | |
| | GGCCCCAGTTCTCCTGGCTCGTGTATGGCATCATCGTCCTGGC | |
| | CCTGACCATCTTCAACGCCTACTCAGAGTACCAGGTCAGCAG | |
| | GTACGTGATGTTCGGCTTCTCCGTGGCCGGAGCAGTGGTGAC | |
| | CTTCGCCCTGTGGATGATGTACTTCGTGAGGAGCATCCAACT | |
| | GTACAGGAGGACCAAAAGCTGGTGGTCCTTCAACCCAGAAA | |
| | CCAACGCCATCCTCTGCGTGAACGCCCTGGGCAGGTCCTACG | |
| | TCCTCCCCCTGGACGGCACCCCCACCGGGGTCACCCTCACCC | |
| | TCCTGTCAGGGAACCTGTACGCTGAGGGCTTCAAGATGGCTG | |
| | GAGGCCTGACAATTGAACACCTGCCCAAGTACGTCATGATCG | |
| | CAACACCCTCCAGAACCATCGTCTACACCCTGGTGGGCAAG | |
| | CAGCTGAAGGCCACCACCGCCACCGGCTGGGCCTACTACGT | |
| | CAAGTCCAAGGCCGGCGACTACAGCACCGAGGCCAGGACCG | |
| | ACAACCTCTCAGAGCACGAGAAGCTGCTGCACATGGTG | |
| 79 | ATGTACCCATACGATGTTCCAGACTACGCTTACCCATATGACG | HA- |
| | TGCCAGACTATGCCTACCCCTACGACGTGCCCGACTACGCAG | M_d(M6) |
| | AGAGGTACTGTGCCATGCAGAACACCGGTTCCCAGTGCATCA | |
| | ACGGCACAGACTCCTCCTGCTCCACCTGCTTCGAGAGAGGA | |
| | GGCCTGATCTGGCACCTGGCAAACTGGAACTTCAGCTGGAG | |
| | CGTGATCCTGATAGTGTTCATAACCGTCCTGAAGTACGGCAG | |
| | ACCACAGTTCTCATGGCTTGTCTATGGCATCATCGTCCTGGCC | |
| | CTGACCATCTTCAACGCCTACTCTGAGTACCAGGTGTCAAGG | |
| | TATGTCATGTTCGGCTTCTCAGTGGCTGGAGCTGTGGTGACC | |
| | TTTGCTCTGTGGATGATGTACTTCGTGAGGTCCATCCAGCTGT | |
| | ACAGGAGGACAAAGTCATGGTGGTCCTTCAACCCAGAAACC | |
| | AATGCCATCCTGTGCGTCAACGCACTGGGCAGAAGCTACGTC | |
| | CTACCACTGGACGGCACTCCTACAGGAGTGACCCTGACCCTG | |
| | CTGTCAGGCAATCTGTACGCAGAGGGGTTCAAGATGGCCGGT | |
| | GGCCTGACCATCGAGCATCTGCCTAAGTACGTGATGATCGCC | |
| | ACCCCTAGCAGGACAATCGTGTACACCCTGGTGGGAAAGCA | |
| | GCTAAAGGCGACCACAGCCACAGGCTGGGCCTACTACGTGA | |
| | AGTCCAAGGCAGGGGACTATTCAACCGAGGCCAGGACCGAC | |
| | AACCTGTCAGAGCACGAGAAGCTGCTGCACATGGTC | |
| 80 | ATGAGATTTGTGATGTCCCCCACTGTCCTGCTGCTGCTCCTGG | MHCIsp- |
| | GAGCCCTGGCAGCCCCCCAGACCTGGGCAGGCAGCTACCCC | HA-M(M2) |
| | TACGATGTGCCAGACTACGCCTACCCCTACGATGTGCCAGAC | |
| | TACGCCTACCCCTACGATGTGCCAGACTACGCCGAGAGATAC | |
| | TGTGCCATGCAGAACACAGGCAGCCAGTGCATCAATGGAAC | |
| | AGACAGCAGCTGCAGCACCTGCTTTGAAAGAGGAGGCCTGA | |
| | TCTGGCACCTGGCCAACTGGAACTTCAGCTGGAGTGTGATCC | |
| | TGATTGTCTTCATTACAGTGCTGAAGTACGGCAGGCCCCAGT | |
| | TCAGCTGGCTGGTGTACGGCATCAAGATGCTCATCATGTGGC | |
| | TGCTGTGGCCCATTGTGTTGGCCCTCACCATCTTCAATGCCTA | |
| | CAGCGAGTACCAGGTGTCCAGATACGTGATGTTCGGCTTCTC | |
| | AGTGGCAGGAGCCGTGGTGACCTTTGCCCTCTGGATGATGTA | |
| | CTTTGTGAGGTCCATCCAGCTCTACAGAAGAACAAAGAGCT | |
| | GGTGGAGCTTCAACCCAGAAACCAACGCCATCCTGTGTGTC | |
| | AATGCCCTGGGCAGATCCTATGTGCTGCCCCTGGATGGCACC | |
| | CCCACAGGCGTCACCCTCACCCTCCTGAGCGGCAACCTGTAC | |
| | GCCGAGGGCTTCAAGATGGCCGGCGGCCTGACAATCGAGCA | |
| | CCTGCCCAAGTATGTGATGATTGCCACCCCCAGCAGGACAAT | |
| | AGTCTACACCCTGGTGGGAAAACAGCTGAAGGCTACCACAG | |
| | CCACAGGCTGGGCCTACTACGTCAAGAGCAAGGCCGGCGAC | |
| | TACAGCACAGAGGCCAGGACCGACAACCTGTCCGAACATGA | |
| | AAAACTCCTGCACATGGTC | |
| 81 | ATGAGATTTGTGATGAGCCCCACTGTGCTGCTGCTGCTGCTG | MHCIsp- |
| | GGAGCCCTGGCAGCCCCCCAGACCTGGGCTGGCTCCTACCC | HA-M(M3) |
| | CTACGACGTGCCCGACTACGCCTACCCCTACGACGTGCCAGA | |
| | CTACGCCTACCCCTACGACGTGCCAGACTACGCCGAGAGGTA | |
| | CTGCGCCATGCAGAACACAGGCTCCCAGTGCATCAACGGAA | |
| | CAGACTCCTCCTGCAGCACCTGCTTCGAGAGAGGAGGCCTC | |
| | ATTTGGCACCTGGCCAACTGGAACTTCAGCTGGTCAGTCATT | |
| | CTGATAGTCTTCATAACAGTGCTGAAGTACGGCAGGCCCCAG | |
| | TTCTCCTGGCTCGTGTATGGCATCAAGATGCTGATCATGTGGC | |
| | TGCTGTGGCCCATCGTCCTGGCCCTGACCATCTTCAACGCCT | |
| | ACTCAGAGTACCAGGTCAGCAGGTACGTGATGTTCGGCTTCT | |
| | CCGTGGCCGGAGCAGTGGTGACCTTCGCCCTGTGGATGATGT | |
| | ACTTCGTGAGGAGCATCCAACTGTACAGGAGGACCAAAAGC | |
| | TGGTGGTCCTTCAACCCAGAAACCAACGCCATCCTCTGCGTG | |
| | AACGCCCTGGGCAGGTCCTACGTCCTCCCCCTGGACGGCAC | |
| | CCCCACCGGGGTCACCCTCACCCTCCTGTCAGGGAACCTGTA | |
| | CGCTGAGGGCTTCAAGATGGCTGGAGGCCTGACAATTGAAC | |
| | ACCTGCCCAAGTACGTCATGATCGCAACACCCTCCAGAACCA | |
| | TCGTCTACACCCTGGTGGGCAAGCAGCTGAAGGCCACCACC | |
| | GCCACCGGCTGGGCCTACTACGTCAAGTCCAAGGCCGGCGA | |
| | CTACAGCACCGAGGCCAGGACCGACAACCTCTCAGAGCACG | |
| | AGAAGCTGCTGCACATGGTG | |
| 82 | ATGAGATTCGTGATGTCCCCTACCGTACTACTACTCCTACTTG | MHCIsp- |
| | GCGCACTAGCAGCTCCTCAAACTTGGGCCGGATCCTACCCAT | HA-M(M6) |
| | ACGATGTTCCAGACTACGCTTACCCATATGACGTGCCAGACT | |
| | ATGCCTACCCCTACGACGTGCCCGACTACGCAGAGAGGTACT | |
| | GTGCCATGCAGAACACCGGTTCCCAGTGCATCAACGGCACA | |
| | GACTCCTCCTGCTCCACCTGCTTCGAGAGAGGAGGCCTGATC | |
| | TGGCACCTGGCAAACTGGAACTTCAGCTGGAGCGTGATCCT | |
| | GATAGTGTTCATAACCGTCCTGAAGTACGGCAGACCACAGTT | |
| | CTCATGGCTTGTCTATGGCATCAAGATGCTGATTATGTGGCTG | |
| | CTTTGGCCTATCGTCCTGGCCCTGACCATCTTCAACGCCTACT | |
| | CTGAGTACCAGGTGTCAAGGTATGTCATGTTCGGCTTCTCAG | |
| | TGGCTGGAGCTGTGGTGACCTTTGCTCTGTGGATGATGTACT | |
| | TCGTGAGGTCCATCCAGCTGTACAGGAGGACAAAGTCATGGT | |
| | GGTCCTTCAACCCAGAAACCAATGCCATCCTGTGCGTCAACG | |
| | CACTGGGCAGAAGCTACGTCCTACCACTGGACGGCACTCCTA | |
| | CAGGAGTGACCCTGACCCTGCTGTCAGGCAATCTGTACGCAG | |
| | AGGGGTTCAAGATGGCCGGTGGCCTGACCATCGAGCATCTGC | |
| | CTAAGTACGTGATGATCGCCACCCCTAGCAGGACAATCGTGT | |
| | ACACCCTGGTGGGAAAGCAGCTAAAGGCGACCACAGCCACA | |
| | GGCTGGGCCTACTACGTGAAGTCCAAGGCAGGGGACTATTC | |
| | AACCGAGGCCAGGACCGACAACCTGTCAGAGCACGAGAAG | |
| | CTGCTGCACATGGTC | |
| 83 | ATGAGATTTGTGATGTCCCCCACTGTCCTGCTGCTGCTCCTGG | MHCIsp- |
| | GAGCCCTGGCAGCCCCCCAGACCTGGGCAGGCAGCTACCCC | HA-M(M2)- |
| | TACGATGTGCCAGACTACGCCTACCCCTACGATGTGCCAGAC | MITD |
| | TACGCCTACCCCTACGATGTGCCAGACTACGCCGAGAGATAC | |
| | TGTGCCATGCAGAACACAGGCAGCCAGTGCATCAATGGAAC | |
| | AGACAGCAGCTGCAGCACCTGCTTTGAAAGAGGAGGCCTGA | |
| | TCTGGCACCTGGCCAACTGGAACTTCAGCTGGAGTGTGATCC | |
| | TGATTGTCTTCATTACAGTGCTGAAGTACGGCAGGCCCCAGT | |
| | TCAGCTGGCTGGTGTACGGCATCAAGATGCTCATCATGTGGC | |
| | TGCTGTGGCCCATTGTGTTGGCCCTCACCATCTTCAATGCCTA | |
| | CAGCGAGTACCAGGTGTCCAGATACGTGATGTTCGGCTTCTC | |
| | AGTGGCAGGAGCCGTGGTGACCTTTGCCCTCTGGATGATGTA | |
| | CTTTGTGAGGTCCATCCAGCTCTACAGAAGAACAAAGAGCT | |
| | GGTGGAGCTTCAACCCAGAAACCAACGCCATCCTGTGTGTC | |
| | AATGCCCTGGGCAGATCCTATGTGCTGCCCCTGGATGGCACC | |
| | CCCACAGGCGTCACCCTCACCCTCCTGAGCGGCAACCTGTAC | |
| | GCCGAGGGCTTCAAGATGGCCGGCGGCCTGACAATCGAGCA | |
| | CCTGCCCAAGTATGTGATGATTGCCACCCCCAGCAGGACAAT | |
| | AGTCTACACCCTGGTGGGAAAACAGCTGAAGGCTACCACAG | |
| | CCACAGGCTGGGCCTACTACGTCAAGAGCAAGGCCGGCGAC | |
| | TACAGCACAGAGGCCAGGACCGACAACCTGTCCGAACATGA | |
| | AAAACTCCTGCACATGGTCTTCCTGGGCATCATCGCTGGCGT | |
| | GGTGGTCCTGGTGGTGACTGTGGTGGTCGGAGCCGTCATCTG | |
| | GAGGAAGAAGTGCAGCGGCAGAAAGGGCCCAAGCTACAGC | |
| | CACGCCGCCAGAGATGACAGCACCCAGGGCAGCGACAGCA | |
| | GCCTCATGGCCCCCAAGGTC | |
| 84 | ATGAGATTTGTGATGAGCCCCACTGTGCTGCTGCTGCTGCTG | MHCIsp- |
| | GGAGCCCTGGCAGCCCCCCAGACCTGGGCTGGCTCCTACCC | HA-M(M3)- |
| | CTACGACGTGCCCGACTACGCCTACCCCTACGACGTGCCAGA | MITD |
| | CTACGCCTACCCCTACGACGTGCCAGACTACGCCGAGAGGTA | |
| | CTGCGCCATGCAGAACACAGGCTCCCAGTGCATCAACGGAA | |
| | CAGACTCCTCCTGCAGCACCTGCTTCGAGAGAGGAGGCCTC | |
| | ATTTGGCACCTGGCCAACTGGAACTTCAGCTGGTCAGTCATT | |
| | CTGATAGTCTTCATAACAGTGCTGAAGTACGGCAGGCCCCAG | |
| | TTCTCCTGGCTCGTGTATGGCATCAAGATGCTGATCATGTGGC | |
| | TGCTGTGGCCCATCGTCCTGGCCCTGACCATCTTCAACGCCT | |
| | ACTCAGAGTACCAGGTCAGCAGGTACGTGATGTTCGGCTTCT | |
| | CCGTGGCCGGAGCAGTGGTGACCTTCGCCCTGTGGATGATGT | |
| | ACTTCGTGAGGAGCATCCAACTGTACAGGAGGACCAAAAGC | |
| | TGGTGGTCCTTCAACCCAGAAACCAACGCCATCCTCTGCGTG | |
| | AACGCCCTGGGCAGGTCCTACGTCCTCCCCCTGGACGGCAC | |
| | CCCCACCGGGGTCACCCTCACCCTCCTGTCAGGGAACCTGTA | |
| | CGCTGAGGGCTTCAAGATGGCTGGAGGCCTGACAATTGAAC | |
| | ACCTGCCCAAGTACGTCATGATCGCAACACCCTCCAGAACCA | |
| | TCGTCTACACCCTGGTGGGCAAGCAGCTGAAGGCCACCACC | |
| | GCCACCGGCTGGGCCTACTACGTCAAGTCCAAGGCCGGCGA | |
| | CTACAGCACCGAGGCCAGGACCGACAACCTCTCAGAGCACG | |
| | AGAAGCTGCTGCACATGGTGTTCCTGGGCATCATCGCAGGAG | |
| | TGGTGGTCCTGGTGGTCACAGTCGTGGTGGGAGCAGTGATCT | |
| | GGAGGAAGAAGTGCTCAGGAAGGAAGGGCCCATCCTACTCC | |
| | CACGCCGCCAGGGACGACTCAACCCAGGGCTCAGACAGCTC | |
| | CCTGATGGCCCCCAAGGTG | |
| 85 | ATGAGATTCGTGATGTCCCCTACCGTACTACTACTCCTACTTG | MHCIsp- |
| | GCGCACTAGCAGCTCCTCAAACTTGGGCCGGATCCTACCCAT | HA-M(M6)- |
| | ACGATGTTCCAGACTACGCTTACCCATATGACGTGCCAGACT | MITD |
| | ATGCCTACCCCTACGACGTGCCCGACTACGCAGAGAGGTACT | |
| | GTGCCATGCAGAACACCGGTTCCCAGTGCATCAACGGCACA | |
| | GACTCCTCCTGCTCCACCTGCTTCGAGAGAGGAGGCCTGATC | |
| | TGGCACCTGGCAAACTGGAACTTCAGCTGGAGCGTGATCCT | |
| | GATAGTGTTCATAACCGTCCTGAAGTACGGCAGACCACAGTT | |
| | CTCATGGCTTGTCTATGGCATCAAGATGCTGATTATGTGGCTG | |
| | CTTTGGCCTATCGTCCTGGCCCTGACCATCTTCAACGCCTACT | |
| | CTGAGTACCAGGTGTCAAGGTATGTCATGTTCGGCTTCTCAG | |
| | TGGCTGGAGCTGTGGTGACCTTTGCTCTGTGGATGATGTACT | |
| | TCGTGAGGTCCATCCAGCTGTACAGGAGGACAAAGTCATGGT | |
| | GGTCCTTCAACCCAGAAACCAATGCCATCCTGTGCGTCAACG | |
| | CACTGGGCAGAAGCTACGTCCTACCACTGGACGGCACTCCTA | |
| | CAGGAGTGACCCTGACCCTGCTGTCAGGCAATCTGTACGCAG | |
| | AGGGGTTCAAGATGGCCGGTGGCCTGACCATCGAGCATCTGC | |
| | CTAAGTACGTGATGATCGCCACCCCTAGCAGGACAATCGTGT | |
| | ACACCCTGGTGGGAAAGCAGCTAAAGGCGACCACAGCCACA | |
| | GGCTGGGCCTACTACGTGAAGTCCAAGGCAGGGGACTATTC | |
| | AACCGAGGCCAGGACCGACAACCTGTCAGAGCACGAGAAG | |
| | CTGCTGCACATGGTCTTCCTGGGCATCATCGCAGGAGTGGTG | |
| | GTGCTGGTGGTGACCGTGGTGGTGGGGGCTGTAATCTGGAG | |
| | GAAGAAGTGCTCAGGGAGAAAGGGCCCAAGCTACTCTCACG | |
| | CCGCCAGGGATGACTCCACACAGGGCTCAGACTCCTCACTG | |
| | ATGGCTCCAAAGGTC | |
| 86 | ATGCACCACCACCACCACCACGCCACACAGGGCCAGAGGGT | His-N(N2) |
| | GAACTGGGGCGACGAGCCATCCAAGAGGAGGGGAAGGAGC | |
| | AACAGCAGAGGAAGGAAGAACAACACCATCCCCCTGTCCTT | |
| | CTTCAACCCAATTCAGCTAGAGCCAGGCAGCAAGTTCTGGTC | |
| | AGTGTGCCCCAGAGACTTCGTGCCCAAGGGCATCGGAAACA | |
| | AGGACCAGCAGATCGGCTACTGGAACAGACAGGAGAGATAC | |
| | AGAATTGTGAAAGGCCAGAGAAAGGAGCTGCCAGAGAGGT | |
| | GGTTCTTCTACTTCCTGGGCACCGGCCCACAGGCAGACGCCA | |
| | AGTTCAAGGACAAGATCGATGGAGTGTTCTGGGTGGCCAAG | |
| | GACGGCGCCATGAACAAGCCCACCACACTGGGCACAAGAGG | |
| | AACAAACAATGAGAGCAAGCCACTGAAGTTTGATGGCAAGA | |
| | TCCCACCCCAGTTCCAGCTGGAGGTGAACAGGAGCAGAAAC | |
| | AACAGCAGAAGCGGCAGCCAGAGCAGAAGTGTGAGCAGAA | |
| | ACAGAAGCCAGAGCAGAGGAAGACAGCAGAGCAACAACCA | |
| | GAACAACGTGGAGGACACCATCGTGGCCGTGCTGCAGAAGC | |
| | TGGGGGTCACAGAAAAGCAGAGGAGCAGAAGCAAGAGCAG | |
| | GGACAGAGGAGACAGCAAGCCAAGAGACACCACCCCCAAC | |
| | AACGCCAACAAGCACACCTGGAAGAAGACAGCCGGCAAGG | |
| | GAGATGTGACCAACTTCTACGGCGCCAGAAGCGCCAGCGCC | |
| | AACTTCGGAGACTCAGACCTGGTGGCCAATGGAAACGCAGC | |
| | CAAGAGCTACCCCCAGATCGCAGAGTGTGTGCCCTCTGTCTC | |
| | CAGCATGCTGTTTGGCAGCCAGTGGAGCGCCGAGGACGACG | |
| | GTGACCAGGTGAAGGTGACCCTGACACACACATACTACCTG | |
| | CCCAAAGATGACGCCAAGACCAGCCAGTTCCTGGAGCAGAT | |
| | TGATGCCTACAAGAGGCCCAGCCAGGTGGCCAAGGACCAGA | |
| | GACAGAGGAAGAGCAGGTCCAAGAGCGCCGAGAAGAAGCC | |
| | AGAAGAATTGAGTGTCACCCTGGTGGAGGCCTACACAGACG | |
| | TGTTTGATGACACCCAGGTGGAGATGATTGATGAGGTGACCA | |
| | AC | |
| 87 | ATGCACCACCACCACCACCACGCCACCCAGGGCCAGAGAGT | His-N(N3) |
| | GAACTGGGGCGACGAGCCCTCAAAAAGGAGGGGCAGATCC | |
| | AACAGCAGAGGCAGGAAGAACAACACCATCCCCCTGAGCTT | |
| | CTTCAACCCCATCCAGCTGGAGCCAGGCTCCAAGTTCTGGTC | |
| | AGTGTGCCCAAGGGACTTCGTGCCCAAGGGCATCGGCAACA | |
| | AGGACCAGCAGATCGGCTACTGGAACAGGCAGGAGAGATAC | |
| | AGAATCGTGAAGGGCCAGAGGAAGGAACTGCCAGAAAGGT | |
| | GGTTCTTCTACTTCCTGGGCACCGGCCCCCAGGCTGACGCCA | |
| | AGTTCAAAGACAAGATCGACGGGGTGTTCTGGGTGGCCAAG | |
| | GACGGCGCCATGAACAAGCCAACAACACTGGGCACCAGAG | |
| | GAACCAACAACGAGAGCAAGCCACTGAAGTTTGACGGCAA | |
| | GATCCCCCCCCAGTTCCAGCTGGAAGTCAACAGGAGCAGGA | |
| | ACAACAGCAGGTCCGGCTCACAAAGCAGGAGCGTGTCCAGA | |
| | AACAGATCCCAGTCAAGAGGAAGACAGCAGTCCAACAACCA | |
| | GAACAACGTGGAGGACACCATAGTGGCCGTGCTGCAGAAGC | |
| | TGGGAGTCACAGAGAAGCAGAGGAGCAGATCCAAGTCCAG | |
| | GGACAGGGGAGACAGCAAGCCCAGGGACACCACACCCAAC | |
| | AACGCCAACAAGCACACCTGGAAGAAGACAGCCGGCAAGG | |
| | GAGATGTGACCAACTTCTACGGCGCCAGAAGCGCCTCAGCC | |
| | AACTTCGGAGACTCAGACCTGGTGGCCAACGGAAACGCCGC | |
| | CAAGAGCTACCCCCAGATCGCCGAATGTGTCCCCTCAGTGTC | |
| | CTCCATGCTCTTCGGCTCACAGTGGTCAGCAGAGGACGACG | |
| | GCGACCAGGTGAAGGTGACCCTGACCCACACCTACTACCTG | |
| | CCCAAGGACGACGCCAAGACAAGCCAGTTCCTGGAGCAGAT | |
| | CGACGCCTACAAGAGGCCATCCCAGGTGGCCAAGGACCAGA | |
| | GGCAGAGGAAGAGCAGAAGCAAGTCAGCCGAGAAGAAACC | |
| | AGAGGAGCTGTCAGTCACCCTGGTGGAGGCCTACACCGACG | |
| | TGTTCGACGACACCCAGGTGGAGATGATCGACGAGGTGACC | |
| | AAC | |
| 88 | ATGCATCATCATCATCACCACGCAACACAAGGACAGAGAGTA | His-N(N6) |
| | AATTGGGGGGATGAGCCCAGCAAGAGGCGAGGCAGAAGCA | |
| | ACTCAAGAGGGAGAAAAAACAATACCATCCCACTGTCATTCT | |
| | TCAACCCCATTCAACTGGAGCCAGGCTCTAAATTCTGGAGTG | |
| | TATGCCCCAGGGACTTTGTGCCCAAGGGCATAGGGAACAAG | |
| | GACCAGCAAATAGGATACTGGAACCGGCAGGAGAGATACAG | |
| | AATTGTCAAGGGTCAGAGAAAGGAGCTGCCAGAGAGATGGT | |
| | TCTTCTACTTCCTAGGAACAGGCCCACAGGCAGACGCTAAGT | |
| | TCAAGGATAAGATCGATGGTGTCTTCTGGGTCGCCAAGGATG | |
| | GTGCAATGAATAAACCAACCACCCTGGGGACCAGGGGGACA | |
| | AACAATGAGTCCAAGCCCCTCAAGTTTGATGGCAAAATCCCC | |
| | CCACAGTTCCAGCTGGAGGTCAACAGGAGCAGGAACAATAG | |
| | CCGTTCAGGGTCCCAGTCCAGATCTGTGTCCAGAAACAGGTC | |
| | CCAGAGCAGGGGACGGCAGCAGAGTAACAACCAGAATAATG | |
| | TGGAAGACACCATAGTAGCAGTGCTCCAGAAACTGGGGGTC | |
| | ACAGAAAAACAGAGGAGCAGGTCCAAGTCTAGGGACCGTG | |
| | GGGACTCTAAGCCAAGGGACACCACACCCAACAACGCCAAC | |
| | AAGCACACATGGAAAAAAACAGCAGGGAAGGGTGATGTCA | |
| | CCAACTTTTACGGGGCCAGGTCAGCCTCTGCAAACTTCGGGG | |
| | ATAGTGACCTGGTGGCCAACGGCAATGCTGCTAAATCCTACC | |
| | CTCAGATTGCTGAGTGCGTACCCTCTGTATCCTCTATGCTCTT | |
| | TGGCTCACAATGGTCTGCTGAGGATGATGGTGACCAGGTCAA | |
| | GGTCACCTTGACCCATACCTACTATCTGCCCAAGGATGATGCA | |
| | AAAACCAGCCAGTTCCTAGAGCAGATAGATGCCTACAAGAG | |
| | GCCCAGCCAGGTGGCCAAGGATCAGAGGCAGAGAAAGAGC | |
| | AGATCCAAGAGCGCAGAAAAGAAACCAGAGGAGTTATCTGT | |
| | GACCCTGGTGGAGGCCTACACAGATGTCTTTGATGATACACA | |
| | GGTGGAAATGATAGATGAGGTGACTAAC | |
| 89 | ATGCACCACCACCACCACCACGCCACACAGGGCCAGAGGGT | His- |
| | GAACTGGGGCGACGAGCCATCCAAGAGGAGGGGAAGGAGC | N_d(N2) |
| | AACAGCAGAGGAAGGAAGAACAACACCATCCCCCTGTCCTT | |
| | CTTCAACCCAATTCAGCTAGAGCCAGGCAGCAAGTTCTGGTC | |
| | AGTGTGCCCCAGAGACTTCGTGCCCAAGGGCATCGGAAACA | |
| | AGGACCAGCAGATCGGCTACTGGAACAGACAGGAGAGATAC | |
| | AGAATTGTGAAAGGCCAGAGAAAGGAGCTGCCAGAGAGGT | |
| | GGTTCTTCTACTTCCTGGGCACCGGCCCACAGGCAGACGCCA | |
| | AGTTCAAGGACAAGATCGATGGAGTGTTCTGGGTGGCCAAG | |
| | GACGGCGCCATGAACAAGCCCACCACACTGGGCACAAGAGG | |
| | AACAAACAATGAGAGCAAGCCACTGAAGTTTGATGGCAAGA | |
| | TCCCACCCCAGTTCCAGCTGGAGGTGAACAGGAGCAGAAGT | |
| | GTGAGCAGAAACAGAAGCCAGAGCAGAGGAAGACAGCAGA | |
| | GCAACAACCAGAACAACGTGGAGGACACCATCGTGGCCGTG | |
| | CTGCAGAAGCTGGGGGTCACAGAAAAGCAGAGGAGCAGAA | |
| | GCAAGAGCAGGGACAGAGGAGACAGCAAGCCAAGAGACAC | |
| | CACCCCCAACAACGCCAACAAGCACACCTGGAAGAAGACA | |
| | GCCGGCAAGGGAGATGTGACCAACTTCTACGGCGCCAGAAG | |
| | CGCCAGCGCCAACTTCGGAGACTCAGACCTGGTGGCCAATG | |
| | GAAACGCAGCCAAGAGCTACCCCCAGATCGCAGAGTGTGTG | |
| | CCCTCTGTCTCCAGCATGCTGTTTGGCAGCCAGTGGAGCGCC | |
| | GAGGACGACGGTGACCAGGTGAAGGTGACCCTGACACACA | |
| | CATACTACCTGCCCAAAGATGACGCCAAGACCAGCCAGTTCC | |
| | TGGAGCAGATTGATGCCTACAAGAGGCCCAGCCAGGTGGCC | |
| | AAGGACCAGAGACAGAGGAAGAGCAGGTCCAAGAGCGCCG | |
| | AGAAGAAGCCAGAAGAATTGAGTGTCACCCTGGTGGAGGCC | |
| | TACACAGACGTGTTTGATGACACCCAGGTGGAGATGATTGAT | |
| | GAGGTGACCAAC | |
| 90 | ATGCACCACCACCACCACCACGCCACCCAGGGCCAGAGAGT | His- |
| | GAACTGGGGCGACGAGCCCTCAAAAAGGAGGGGCAGATCC | N_d(N3) |
| | AACAGCAGAGGCAGGAAGAACAACACCATCCCCCTGAGCTT | |
| | CTTCAACCCCATCCAGCTGGAGCCAGGCTCCAAGTTCTGGTC | |
| | AGTGTGCCCAAGGGACTTCGTGCCCAAGGGCATCGGCAACA | |
| | AGGACCAGCAGATCGGCTACTGGAACAGGCAGGAGAGATAC | |
| | AGAATCGTGAAGGGCCAGAGGAAGGAACTGCCAGAAAGGT | |
| | GGTTCTTCTACTTCCTGGGCACCGGCCCCCAGGCTGACGCCA | |
| | AGTTCAAAGACAAGATCGACGGGGTGTTCTGGGTGGCCAAG | |
| | GACGGCGCCATGAACAAGCCAACAACACTGGGCACCAGAG | |
| | GAACCAACAACGAGAGCAAGCCACTGAAGTTTGACGGCAA | |
| | GATCCCCCCCCAGTTCCAGCTGGAAGTCAACAGGAGCAGGA | |
| | GCGTGTCCAGAAACAGATCCCAGTCAAGAGGAAGACAGCAG | |
| | TCCAACAACCAGAACAACGTGGAGGACACCATAGTGGCCGT | |
| | GCTGCAGAAGCTGGGAGTCACAGAGAAGCAGAGGAGCAGA | |
| | TCCAAGTCCAGGGACAGGGGAGACAGCAAGCCCAGGGACA | |
| | CCACACCCAACAACGCCAACAAGCACACCTGGAAGAAGAC | |
| | AGCCGGCAAGGGAGATGTGACCAACTTCTACGGCGCCAGAA | |
| | GCGCCTCAGCCAACTTCGGAGACTCAGACCTGGTGGCCAAC | |
| | GGAAACGCCGCCAAGAGCTACCCCCAGATCGCCGAATGTGT | |
| | CCCCTCAGTGTCCTCCATGCTCTTCGGCTCACAGTGGTCAGC | |
| | AGAGGACGACGGCGACCAGGTGAAGGTGACCCTGACCCAC | |
| | ACCTACTACCTGCCCAAGGACGACGCCAAGACAAGCCAGTT | |
| | CCTGGAGCAGATCGACGCCTACAAGAGGCCATCCCAGGTGG | |
| | CCAAGGACCAGAGGCAGAGGAAGAGCAGAAGCAAGTCAGC | |
| | CGAGAAGAAACCAGAGGAGCTGTCAGTCACCCTGGTGGAG | |
| | GCCTACACCGACGTGTTCGACGACACCCAGGTGGAGATGATC | |
| | GACGAGGTGACCAAC | |
| 91 | ATGCATCATCATCATCACCACGCAACACAAGGACAGAGAGTA | His- |
| | AATTGGGGGGATGAGCCCAGCAAGAGGCGAGGCAGAAGCA | N_d(N6) |
| | ACTCAAGAGGGAGAAAAAACAATACCATCCCACTGTCATTCT | |
| | TCAACCCCATTCAACTGGAGCCAGGCTCTAAATTCTGGAGTG | |
| | TATGCCCCAGGGACTTTGTGCCCAAGGGCATAGGGAACAAG | |
| | GACCAGCAAATAGGATACTGGAACCGGCAGGAGAGATACAG | |
| | AATTGTCAAGGGTCAGAGAAAGGAGCTGCCAGAGAGATGGT | |
| | TCTTCTACTTCCTAGGAACAGGCCCACAGGCAGACGCTAAGT | |
| | TCAAGGATAAGATCGATGGTGTCTTCTGGGTCGCCAAGGATG | |
| | GTGCAATGAATAAACCAACCACCCTGGGGACCAGGGGGACA | |
| | AACAATGAGTCCAAGCCCCTCAAGTTTGATGGCAAAATCCCC | |
| | CCACAGTTCCAGCTGGAGGTCAACAGGAGCAGGTCTGTGTC | |
| | CAGAAACAGGTCCCAGAGCAGGGGACGGCAGCAGAGTAAC | |
| | AACCAGAATAATGTGGAAGACACCATAGTAGCAGTGCTCCAG | |
| | AAACTGGGGGTCACAGAAAAACAGAGGAGCAGGTCCAAGT | |
| | CTAGGGACCGTGGGGACTCTAAGCCAAGGGACACCACACCC | |
| | AACAACGCCAACAAGCACACATGGAAAAAAACAGCAGGGA | |
| | AGGGTGATGTCACCAACTTTTACGGGGCCAGGTCAGCCTCTG | |
| | CAAACTTCGGGGATAGTGACCTGGTGGCCAACGGCAATGCT | |
| | GCTAAATCCTACCCTCAGATTGCTGAGTGCGTACCCTCTGTAT | |
| | CCTCTATGCTCTTTGGCTCACAATGGTCTGCTGAGGATGATGG | |
| | TGACCAGGTCAAGGTCACCTTGACCCATACCTACTATCTGCC | |
| | CAAGGATGATGCAAAAACCAGCCAGTTCCTAGAGCAGATAG | |
| | ATGCCTACAAGAGGCCCAGCCAGGTGGCCAAGGATCAGAGG | |
| | CAGAGAAAGAGCAGATCCAAGAGCGCAGAAAAGAAACCAG | |
| | AGGAGTTATCTGTGACCCTGGTGGAGGCCTACACAGATGTCT | |
| | TTGATGATACACAGGTGGAAATGATAGATGAGGTGACTAAC | |
| | ATGAGATTTGTGATGTCCCCCACTGTCCTGCTGCTGCTCCTGG | |
| 92 | GAGCCCTGGCAGCCCCCCAGACCTGGGCAGGCAGCCACCAC | MHCIsp- |
| | CACCACCACCACGCCACACAGGGCCAGAGGGTGAACTGGG | His-N(N2) |
| | GCGACGAGCCATCCAAGAGGAGGGGAAGGAGCAACAGCAG | |
| | AGGAAGGAAGAACAACACCATCCCCCTGTCCTTCTTCAACC | |
| | CAATTCAGCTAGAGCCAGGCAGCAAGTTCTGGTCAGTGTGCC | |
| | CCAGAGACTTCGTGCCCAAGGGCATCGGAAACAAGGACCAG | |
| | CAGATCGGCTACTGGAACAGACAGGAGAGATACAGAATTGT | |
| | GAAAGGCCAGAGAAAGGAGCTGCCAGAGAGGTGGTTCTTCT | |
| | ACTTCCTGGGCACCGGCCCACAGGCAGACGCCAAGTTCAAG | |
| | GACAAGATCGATGGAGTGTTCTGGGTGGCCAAGGACGGCGC | |
| | CATGAACAAGCCCACCACACTGGGCACAAGAGGAACAAAC | |
| | AATGAGAGCAAGCCACTGAAGTTTGATGGCAAGATCCCACC | |
| | CCAGTTCCAGCTGGAGGTGAACAGGAGCAGAAACAACAGC | |
| | AGAAGCGGCAGCCAGAGCAGAAGTGTGAGCAGAAACAGAA | |
| | GCCAGAGCAGAGGAAGACAGCAGAGCAACAACCAGAACAA | |
| | CGTGGAGGACACCATCGTGGCCGTGCTGCAGAAGCTGGGGG | |
| | TCACAGAAAAGCAGAGGAGCAGAAGCAAGAGCAGGGACAG | |
| | AGGAGACAGCAAGCCAAGAGACACCACCCCCAACAACGCC | |
| | AACAAGCACACCTGGAAGAAGACAGCCGGCAAGGGAGATG | |
| | TGACCAACTTCTACGGCGCCAGAAGCGCCAGCGCCAACTTC | |
| | GGAGACTCAGACCTGGTGGCCAATGGAAACGCAGCCAAGAG | |
| | CTACCCCCAGATCGCAGAGTGTGTGCCCTCTGTCTCCAGCAT | |
| | GCTGTTTGGCAGCCAGTGGAGCGCCGAGGACGACGGTGACC | |
| | AGGTGAAGGTGACCCTGACACACACATACTACCTGCCCAAA | |
| | GATGACGCCAAGACCAGCCAGTTCCTGGAGCAGATTGATGC | |
| | CTACAAGAGGCCCAGCCAGGTGGCCAAGGACCAGAGACAG | |
| | AGGAAGAGCAGGTCCAAGAGCGCCGAGAAGAAGCCAGAAG | |
| | AATTGAGTGTCACCCTGGTGGAGGCCTACACAGACGTGTTTG | |
| | ATGACACCCAGGTGGAGATGATTGATGAGGTGACCAAC | |
| 93 | ATGAGATTTGTGATGAGCCCCACTGTGCTGCTGCTGCTGCTG | MHCIsp- |
| | GGAGCCCTGGCAGCCCCCCAGACCTGGGCTGGCTCCCACCA | His-N(N3) |
| | CCACCACCACCACGCCACCCAGGGCCAGAGAGTGAACTGGG | |
| | GCGACGAGCCCTCAAAAAGGAGGGGCAGATCCAACAGCAG | |
| | AGGCAGGAAGAACAACACCATCCCCCTGAGCTTCTTCAACC | |
| | CCATCCAGCTGGAGCCAGGCTCCAAGTTCTGGTCAGTGTGCC | |
| | CAAGGGACTTCGTGCCCAAGGGCATCGGCAACAAGGACCAG | |
| | CAGATCGGCTACTGGAACAGGCAGGAGAGATACAGAATCGT | |
| | GAAGGGCCAGAGGAAGGAACTGCCAGAAAGGTGGTTCTTCT | |
| | ACTTCCTGGGCACCGGCCCCCAGGCTGACGCCAAGTTCAAA | |
| | GACAAGATCGACGGGGTGTTCTGGGTGGCCAAGGACGGCGC | |
| | CATGAACAAGCCAACAACACTGGGCACCAGAGGAACCAAC | |
| | AACGAGAGCAAGCCACTGAAGTTTGACGGCAAGATCCCCCC | |
| | CCAGTTCCAGCTGGAAGTCAACAGGAGCAGGAACAACAGC | |
| | AGGTCCGGCTCACAAAGCAGGAGCGTGTCCAGAAACAGATC | |
| | CCAGTCAAGAGGAAGACAGCAGTCCAACAACCAGAACAAC | |
| | GTGGAGGACACCATAGTGGCCGTGCTGCAGAAGCTGGGAGT | |
| | CACAGAGAAGCAGAGGAGCAGATCCAAGTCCAGGGACAGG | |
| | GGAGACAGCAAGCCCAGGGACACCACACCCAACAACGCCA | |
| | ACAAGCACACCTGGAAGAAGACAGCCGGCAAGGGAGATGT | |
| | GACCAACTTCTACGGCGCCAGAAGCGCCTCAGCCAACTTCG | |
| | GAGACTCAGACCTGGTGGCCAACGGAAACGCCGCCAAGAG | |
| | CTACCCCCAGATCGCCGAATGTGTCCCCTCAGTGTCCTCCAT | |
| | GCTCTTCGGCTCACAGTGGTCAGCAGAGGACGACGGCGACC | |
| | AGGTGAAGGTGACCCTGACCCACACCTACTACCTGCCCAAG | |
| | GACGACGCCAAGACAAGCCAGTTCCTGGAGCAGATCGACGC | |
| | CTACAAGAGGCCATCCCAGGTGGCCAAGGACCAGAGGCAGA | |
| | GGAAGAGCAGAAGCAAGTCAGCCGAGAAGAAACCAGAGGA | |
| | GCTGTCAGTCACCCTGGTGGAGGCCTACACCGACGTGTTCGA | |
| | CGACACCCAGGTGGAGATGATCGACGAGGTGACCAAC | |
| 94 | ATGCGATTCGTCATGTCACCAACCGTTTTACTATTATTACTAGG | MHCIsp- |
| | AGCATTAGCAGCACCGCAAACATGGGCAGGAAGTCATCATCA | His-N(N6) |
| | TCATCACCACGCAACACAAGGACAGAGAGTAAATTGGGGGG | |
| | ATGAGCCCAGCAAGAGGCGAGGCAGAAGCAACTCAAGAGG | |
| | GAGAAAAAACAATACCATCCCACTGTCATTCTTCAACCCCAT | |
| | TCAACTGGAGCCAGGCTCTAAATTCTGGAGTGTATGCCCCAG | |
| | GGACTTTGTGCCCAAGGGCATAGGGAACAAGGACCAGCAAA | |
| | TAGGATACTGGAACCGGCAGGAGAGATACAGAATTGTCAAG | |
| | GGTCAGAGAAAGGAGCTGCCAGAGAGATGGTTCTTCTACTT | |
| | CCTAGGAACAGGCCCACAGGCAGACGCTAAGTTCAAGGATA | |
| | AGATCGATGGTGTCTTCTGGGTCGCCAAGGATGGTGCAATGA | |
| | ATAAACCAACCACCCTGGGGACCAGGGGGACAAACAATGAG | |
| | TCCAAGCCCCTCAAGTTTGATGGCAAAATCCCCCCACAGTTC | |
| | CAGCTGGAGGTCAACAGGAGCAGGAACAATAGCCGTTCAGG | |
| | GTCCCAGTCCAGATCTGTGTCCAGAAACAGGTCCCAGAGCA | |
| | GGGGACGGCAGCAGAGTAACAACCAGAATAATGTGGAAGAC | |
| | ACCATAGTAGCAGTGCTCCAGAAACTGGGGGTCACAGAAAA | |
| | ACAGAGGAGCAGGTCCAAGTCTAGGGACCGTGGGGACTCTA | |
| | AGCCAAGGGACACCACACCCAACAACGCCAACAAGCACAC | |
| | ATGGAAAAAAACAGCAGGGAAGGGTGATGTCACCAACTTTT | |
| | ACGGGGCCAGGTCAGCCTCTGCAAACTTCGGGGATAGTGAC | |
| | CTGGTGGCCAACGGCAATGCTGCTAAATCCTACCCTCAGATT | |
| | GCTGAGTGCGTACCCTCTGTATCCTCTATGCTCTTTGGCTCAC | |
| | AATGGTCTGCTGAGGATGATGGTGACCAGGTCAAGGTCACCT | |
| | TGACCCATACCTACTATCTGCCCAAGGATGATGCAAAAACCA | |
| | GCCAGTTCCTAGAGCAGATAGATGCCTACAAGAGGCCCAGCC | |
| | AGGTGGCCAAGGATCAGAGGCAGAGAAAGAGCAGATCCAA | |
| | GAGCGCAGAAAAGAAACCAGAGGAGTTATCTGTGACCCTGG | |
| | TGGAGGCCTACACAGATGTCTTTGATGATACACAGGTGGAAA | |
| | TGATAGATGAGGTGACTAAC | |
| 95 | ATGAGATTTGTGATGTCCCCCACTGTCCTGCTGCTGCTCCTGG | MHCIsp- |
| | GAGCCCTGGCAGCCCCCCAGACCTGGGCAGGCAGCCACCAC | His-N(N2)- |
| | CACCACCACCACGCCACACAGGGCCAGAGGGTGAACTGGG | MITD |
| | GCGACGAGCCATCCAAGAGGAGGGGAAGGAGCAACAGCAG | |
| | AGGAAGGAAGAACAACACCATCCCCCTGTCCTTCTTCAACC | |
| | CAATTCAGCTAGAGCCAGGCAGCAAGTTCTGGTCAGTGTGCC | |
| | CCAGAGACTTCGTGCCCAAGGGCATCGGAAACAAGGACCAG | |
| | CAGATCGGCTACTGGAACAGACAGGAGAGATACAGAATTGT | |
| | GAAAGGCCAGAGAAAGGAGCTGCCAGAGAGGTGGTTCTTCT | |
| | ACTTCCTGGGCACCGGCCCACAGGCAGACGCCAAGTTCAAG | |
| | GACAAGATCGATGGAGTGTTCTGGGTGGCCAAGGACGGCGC | |
| | CATGAACAAGCCCACCACACTGGGCACAAGAGGAACAAAC | |
| | AATGAGAGCAAGCCACTGAAGTTTGATGGCAAGATCCCACC | |
| | CCAGTTCCAGCTGGAGGTGAACAGGAGCAGAAACAACAGC | |
| | AGAAGCGGCAGCCAGAGCAGAAGTGTGAGCAGAAACAGAA | |
| | GCCAGAGCAGAGGAAGACAGCAGAGCAACAACCAGAACAA | |
| | CGTGGAGGACACCATCGTGGCCGTGCTGCAGAAGCTGGGGG | |
| | TCACAGAAAAGCAGAGGAGCAGAAGCAAGAGCAGGGACAG | |
| | AGGAGACAGCAAGCCAAGAGACACCACCCCCAACAACGCC | |
| | AACAAGCACACCTGGAAGAAGACAGCCGGCAAGGGAGATG | |
| | TGACCAACTTCTACGGCGCCAGAAGCGCCAGCGCCAACTTC | |
| | GGAGACTCAGACCTGGTGGCCAATGGAAACGCAGCCAAGAG | |
| | CTACCCCCAGATCGCAGAGTGTGTGCCCTCTGTCTCCAGCAT | |
| | GCTGTTTGGCAGCCAGTGGAGCGCCGAGGACGACGGTGACC | |
| | AGGTGAAGGTGACCCTGACACACACATACTACCTGCCCAAA | |
| | GATGACGCCAAGACCAGCCAGTTCCTGGAGCAGATTGATGC | |
| | CTACAAGAGGCCCAGCCAGGTGGCCAAGGACCAGAGACAG | |
| | AGGAAGAGCAGGTCCAAGAGCGCCGAGAAGAAGCCAGAAG | |
| | AATTGAGTGTCACCCTGGTGGAGGCCTACACAGACGTGTTTG | |
| | ATGACACCCAGGTGGAGATGATTGATGAGGTGACCAACTTCC | |
| | TGGGCATCATCGCAGGAGTGGTGGTGCTGGTGGTGACAGTG | |
| | GTGGTGGGAGCAGTGATCTGGAGAAAGAAATGCAGCGGGAG | |
| | AAAGGGCCCCAGCTACAGCCACGCCGCCAGGGACGACAGC | |
| | ACCCAGGGCAGCGACAGCAGCCTCATGGCCCCCAAGGTG | |
| 96 | ATGAGATTTGTGATGAGCCCCACTGTGCTGCTGCTGCTGCTG | MHCIsp- |
| | GGAGCCCTGGCAGCCCCCCAGACCTGGGCTGGCTCCCACCA | His-N(N3)- |
| | CCACCACCACCACGCCACCCAGGGCCAGAGAGTGAACTGGG | MITD |
| | GCGACGAGCCCTCAAAAAGGAGGGGCAGATCCAACAGCAG | |
| | AGGCAGGAAGAACAACACCATCCCCCTGAGCTTCTTCAACC | |
| | CCATCCAGCTGGAGCCAGGCTCCAAGTTCTGGTCAGTGTGCC | |
| | CAAGGGACTTCGTGCCCAAGGGCATCGGCAACAAGGACCAG | |
| | CAGATCGGCTACTGGAACAGGCAGGAGAGATACAGAATCGT | |
| | GAAGGGCCAGAGGAAGGAACTGCCAGAAAGGTGGTTCTTCT | |
| | ACTTCCTGGGCACCGGCCCCCAGGCTGACGCCAAGTTCAAA | |
| | GACAAGATCGACGGGGTGTTCTGGGTGGCCAAGGACGGCGC | |
| | CATGAACAAGCCAACAACACTGGGCACCAGAGGAACCAAC | |
| | AACGAGAGCAAGCCACTGAAGTTTGACGGCAAGATCCCCCC | |
| | CCAGTTCCAGCTGGAAGTCAACAGGAGCAGGAACAACAGC | |
| | AGGTCCGGCTCACAAAGCAGGAGCGTGTCCAGAAACAGATC | |
| | CCAGTCAAGAGGAAGACAGCAGTCCAACAACCAGAACAAC | |
| | GTGGAGGACACCATAGTGGCCGTGCTGCAGAAGCTGGGAGT | |
| | CACAGAGAAGCAGAGGAGCAGATCCAAGTCCAGGGACAGG | |
| | GGAGACAGCAAGCCCAGGGACACCACACCCAACAACGCCA | |
| | ACAAGCACACCTGGAAGAAGACAGCCGGCAAGGGAGATGT | |
| | GACCAACTTCTACGGCGCCAGAAGCGCCTCAGCCAACTTCG | |
| | GAGACTCAGACCTGGTGGCCAACGGAAACGCCGCCAAGAG | |
| | CTACCCCCAGATCGCCGAATGTGTCCCCTCAGTGTCCTCCAT | |
| | GCTCTTCGGCTCACAGTGGTCAGCAGAGGACGACGGCGACC | |
| | AGGTGAAGGTGACCCTGACCCACACCTACTACCTGCCCAAG | |
| | GACGACGCCAAGACAAGCCAGTTCCTGGAGCAGATCGACGC | |
| | CTACAAGAGGCCATCCCAGGTGGCCAAGGACCAGAGGCAGA | |
| | GGAAGAGCAGAAGCAAGTCAGCCGAGAAGAAACCAGAGGA | |
| | GCTGTCAGTCACCCTGGTGGAGGCCTACACCGACGTGTTCGA | |
| | CGACACCCAGGTGGAGATGATCGACGAGGTGACCAACTTCC | |
| | TGGGCATCATCGCCGGCGTGGTGGTCCTGGTGGTGACCGTGG | |
| | TGGTCGGAGCAGTGATCTGGAGGAAGAAGTGCTCAGGCAGG | |
| | AAGGGCCCATCCTACAGCCACGCCGCCAGAGATGACAGCAC | |
| | CCAAGGCTCAGACAGCTCCCTGATGGCCCCCAAGGTG | |
| 97 | ATGCGATTCGTCATGTCACCAACCGTTTTACTATTATTACTAGG | MHCIsp- |
| | AGCATTAGCAGCACCGCAAACATGGGCAGGAAGTCATCATCA | His-N(N6)- |
| | TCATCACCACGCAACACAAGGACAGAGAGTAAATTGGGGGG | MITD |
| | ATGAGCCCAGCAAGAGGCGAGGCAGAAGCAACTCAAGAGG | |
| | GAGAAAAAACAATACCATCCCACTGTCATTCTTCAACCCCAT | |
| | TCAACTGGAGCCAGGCTCTAAATTCTGGAGTGTATGCCCCAG | |
| | GGACTTTGTGCCCAAGGGCATAGGGAACAAGGACCAGCAAA | |
| | TAGGATACTGGAACCGGCAGGAGAGATACAGAATTGTCAAG | |
| | GGTCAGAGAAAGGAGCTGCCAGAGAGATGGTTCTTCTACTT | |
| | CCTAGGAACAGGCCCACAGGCAGACGCTAAGTTCAAGGATA | |
| | AGATCGATGGTGTCTTCTGGGTCGCCAAGGATGGTGCAATGA | |
| | ATAAACCAACCACCCTGGGGACCAGGGGGACAAACAATGAG | |
| | TCCAAGCCCCTCAAGTTTGATGGCAAAATCCCCCCACAGTTC | |
| | CAGCTGGAGGTCAACAGGAGCAGGAACAATAGCCGTTCAGG | |
| | GTCCCAGTCCAGATCTGTGTCCAGAAACAGGTCCCAGAGCA | |
| | GGGGACGGCAGCAGAGTAACAACCAGAATAATGTGGAAGAC | |
| | ACCATAGTAGCAGTGCTCCAGAAACTGGGGGTCACAGAAAA | |
| | ACAGAGGAGCAGGTCCAAGTCTAGGGACCGTGGGGACTCTA | |
| | AGCCAAGGGACACCACACCCAACAACGCCAACAAGCACAC | |
| | ATGGAAAAAAACAGCAGGGAAGGGTGATGTCACCAACTTTT | |
| | ACGGGGCCAGGTCAGCCTCTGCAAACTTCGGGGATAGTGAC | |
| | CTGGTGGCCAACGGCAATGCTGCTAAATCCTACCCTCAGATT | |
| | GCTGAGTGCGTACCCTCTGTATCCTCTATGCTCTTTGGCTCAC | |
| | AATGGTCTGCTGAGGATGATGGTGACCAGGTCAAGGTCACCT | |
| | TGACCCATACCTACTATCTGCCCAAGGATGATGCAAAAACCA | |
| | GCCAGTTCCTAGAGCAGATAGATGCCTACAAGAGGCCCAGCC | |
| | AGGTGGCCAAGGATCAGAGGCAGAGAAAGAGCAGATCCAA | |
| | GAGCGCAGAAAAGAAACCAGAGGAGTTATCTGTGACCCTGG | |
| | TGGAGGCCTACACAGATGTCTTTGATGATACACAGGTGGAAA | |
| | TGATAGATGAGGTGACTAACTTCCTTGGAATCATAGCTGGGG | |
| | TCGTTGTCCTCGTAGTGACTGTAGTGGTAGGCGCAGTTATCTG | |
| | GAGGAAGAAATGCTCGGGGAGGAAAGGGCCCTCTTACAGCC | |
| | ATGCTGCCAGGGATGACTCCACACAGGGGTCAGATAGCAGC | |
| | CTCATGGCCCCAAAGGTC | |
| 98 | ATGGACTACAAGGATGATGATGACAAGGACTACAAAGACGA | Flag-S(S2) |
| | CGACGACAAGGACTACAAGGATGACGATGACAAGACCACCA | |
| | ACAATGAATGCATCCAGGTGAACGTGACCCAGCTGGCAGGC | |
| | AATGAAAATTTGATCAGAGACTTCCTGTTCAGCAACTTCAAG | |
| | GAGGAGGGCAGTGTAGTGGTGGGAGGCTACTACCCAACAGA | |
| | GGTGTGGTACAACTGCAGCAGAACAGCCAGAACCACAGCCT | |
| | TCCAGTACTTCAACAACATCCACGCCTTCTACTTTGTGATGGA | |
| | GGCCATGGAAAACAGCACAGGAAATGCCAGAGGAAAACCC | |
| | CTGCTCTTCCACGTGCACGGAGAGCCCGTGTCAGTCATCATC | |
| | AGCGCCTACAGAGATGACGTCCAGCAGCGGCCCCTGCTGAA | |
| | GCATGGACTGGTCTGCATCACCAAGAACAGACACATCAACTA | |
| | CGAGCAGTTCACCAGCAACCAGTGGAACAGCACCTGCACAG | |
| | GAGCAGACAGAAAAATCCCCTTCAGCGTCATCCCCACAGAC | |
| | AACGGCACCAAAATCTATGGCCTGGAGTGGAATGATGACTTT | |
| | GTGACAGCCTATATCAGCGGCAGGAGCTACCACCTCAACATC | |
| | AACACCAACTGGTTCAACAACGTCACCCTGCTCTACTCCAGA | |
| | TCCAGCACAGCCACCTGGGAGTACAGCGCCGCCTATGCCTAC | |
| | CAGGGAGTCTCCAACTTCACCTACTACAAACTGAACAACAC | |
| | CAACGGCCTGAAAACCTACGAGCTGTGTGAGGACTACGAGC | |
| | ACTGCACAGGCTATGCCACAAATGTGTTTGCCCCAACCAGCG | |
| | GAGGCTACATCCCAGACGGCTTCTCCTTCAACAACTGGTTCC | |
| | TCCTCACCAACTCCTCCACATTTGTGAGCGGCAGATTTGTGA | |
| | CCAACCAGCCCCTGCTGATCAACTGCCTGTGGCCCGTGCCCA | |
| | GCTTTGGAGTGGCAGCCCAGGAGTTCTGCTTCGAGGGAGCC | |
| | CAGTTCAGCCAGTGCAACGGAGTCAGCCTGAACAACACAGT | |
| | GGACGTGATCAGATTCAACCTGAACTTCACAGCAGACGTGC | |
| | AGAGTGGAATGGGAGCCACCGTCTTCAGCCTGAACACCACA | |
| | GGAGGAGTGATCCTGGAGATCAGCTGCTACAGCGACACAGT | |
| | GAGCGAGAGCAGCAGCTACAGCTACGGAGAGATCCCATTTG | |
| | GCATCACAGATGGCCCCAGGTACTGCTACGTCCTGTACAATG | |
| | GAACAGCCCTGAAATACCTGGGCACCCTCCCACCCAGCGTG | |
| | AAGGAGATCGCCATCAGCAAGTGGGGCCACTTCTACATCAAT | |
| | GGCTACAACTTCTTCAGCACCTTCCCCATCGGCTGCATCTCCT | |
| | TCAACCTGACCACAGGAGTGAGCGGGGCCTTCTGGACAATC | |
| | GCCTACACATCCTACACAGAAGCCCTGGTGCAGGTGGAGAA | |
| | CACAGCCATCAAAAACGTCACCTACTGCAACAGCCACATCA | |
| | ACAACATCAAGTGCAGCCAGCTGACAGCCAACCTGAACAAC | |
| | GGCTTCTACCCAGTGGCCAGCTCAGAGGTGGGCTTCGTGAA | |
| | CAAGAGCGTGGTGCTCCTGCCCAGCTTCTTCACCTACACAGC | |
| | AGTGAACATCACAATTGACCTGGGCATGAAGCTGAGCGGCTA | |
| | CGGCCAGCCAATTGCCAGCACCCTCTCCAACATCACCCTCCC | |
| | CATGCAGGACAATAACACAGATGTGTACTGCATCAGATCCAA | |
| | CCAGTTCTCTGTCTACGTGCACAGCACCTGCAAAAGCAGCCT | |
| | GTGGGACAACATCTTCAACCAGGACTGCACAGATGTCCTGG | |
| | AGGCCACAGCCGTGATCAAAACAGGCACCTGCCCCTTCAGC | |
| | TTTGACAAACTCAACAACTACCTTACATTCAACAAATTCTGC | |
| | CTCTCCCTCAGCCCAGTGGGAGCCAACTGCAAGTTTGATGTG | |
| | GCCGCCAGGACCAGGACAAATGAACAAGTGGTCAGAAGCCT | |
| | CTACGTCATCTACGAGGAGGGAGACAACATCGTGGGGGTCC | |
| | CCAGCGACAACAGCGGCCTGCACGACCTGAGTGTGCTCCAC | |
| | CTGGACAGCTGCACAGACTACAACATCTACGGCAGGACTGG | |
| | GGTGGGCATCATCAGAAGAACCAACAGCACACTGCTGAGTG | |
| | GCCTGTACTACACCAGCCTGAGTGGAGACTTGCTGGGCTTCA | |
| | AGAATGTGTCAGATGGGGTGATCTACAGTGTGACCCCCTGTG | |
| | ACGTGTCTGCCCAGGCTGCAGTCATCGACGGAGCCATCGTGG | |
| | GAGCCATGACCAGCATTAACAGCGAGCTGCTGGGCCTGACC | |
| | CACTGGACCACCACCCCCAACTTCTACTACTACTCCATCTACA | |
| | ACTACACATCAGAAAGAACAAGAGACACAGCCATCGACAGC | |
| | AATGACGTGGACTGTGAGCCAGTCATCACCTACAGCAACATC | |
| | GGAGTGTGCAAGAACGGAGCCCTGGTGTTCATCAACGTGAC | |
| | CCACAGCGACGGAGATGTCCAGCCCATCAGCACAGGAAATG | |
| | TGACCATCCCAACCAACTTCACCATCAGCGTCCAGGTGGAAT | |
| | ACATGCAGGTGTACACCACCCCAGTGTCCATCGACTGTGCCA | |
| | GATACGTGTGCAATGGAAACCCCAGATGCAACAAGCTCCTCA | |
| | CCCAGTACGTGTCAGCCTGCCAGACAATCGAGCAGGCCCTG | |
| | GCCATGGGAGCCAGGCTCGAGAACATGGAAGTGGACAGCAT | |
| | GCTGTTTGTCTCAGAGAATGCCCTGAAACTGGCCAGCGTGG | |
| | AGGCCTTCAACAGCACAGAGAACCTGGACAGCATCTACAAG | |
| | GAGTGGCCATCAATCGGAGGCAGCTGGCTGGGAGGACTTAA | |
| | GGACATCCTGCCAAGCCACAACAGCAAAAGAAAGTACGGCA | |
| | GCGCCATTGAGGACCTGCTGTTTGACAAGGTGGTCACCTCCG | |
| | GCCTGGGCACAGTGGATGAGGACTACAAGAGATGCACCGGC | |
| | GGCTATGACATTGCCGACCTGGTGTGTGCCCAGTACTACAAT | |
| | GGCATCATGGTGCTGCCTGGAGTGGCCAACGCCGACAAAAT | |
| | GACCATGTACACCGCCTCCCTGGCTGGAGGCATCACACTGGG | |
| | AGCCCTGGGGGGAGGAGCAGTGGCCATCCCCTTTGCAGTGG | |
| | CTGTGCAGGCCAGACTCAACTACGTGGCCCTGCAGACAGAC | |
| | GTGCTCAACAAGAACCAGCAGATCCTGGCCAACGCTTTCAA | |
| | CCAGGCTATCGGAAACATCACCCAGGCCTTTGGAAAAGTGA | |
| | ATGATGCCATCCACCAGACCAGCCAGGGCCTGGCCACAGTG | |
| | GCCAAGGCCCTGGCCAAGGTGCAGGACGTGGTCAACACCCA | |
| | GGGCCAGGCCCTCAGTCACCTCACAGTACAGCTCCAGAACA | |
| | ACTTCCAGGCAATCTCCTCCTCCATCAGCGACATCTACAACA | |
| | GGCTGGACCCCCCAAGCGCTGATGCCCAGGTGGACAGACTG | |
| | ATCACAGGAAGACTCACAGCCCTCAACGCATTTGTGTCCCAG | |
| | ACACTGACCAGGCAGGCAGAGGTCAGGGCCAGCAGGCAGC | |
| | TGGCCAAGGACAAGGTGAATGAGTGCGTGAGGAGCCAGAG | |
| | CCAGAGATTTGGCTTCTGCGGAAACGGCACCCACCTGTTCAG | |
| | CCTGGCCAACGCCGCCCCCAACGGCATGATTTTCTTCCACAC | |
| | AGTCCTCCTCCCCACAGCCTACGAAACAGTGACAGCCTGGT | |
| | CAGGCATCTGTGCCAGCGACGGAGACAGAACCTTTGGCCTG | |
| | GTGGTGAAGGATGTGCAGCTCACCCTCTTCAGAAACCTGGAT | |
| | GACAAGTTCTACCTCACCCCAAGAACCATGTACCAGCCCAGA | |
| | GTGGCCACAAGCAGCGACTTTGTGCAGATTGAGGGCTGTGA | |
| | CGTGCTGTTTGTGAATGCAACAGTGATTGACCTCCCAAGCAT | |
| | CATCCCAGATTACATCGACATCAACCAGACAGTGCAGGACAT | |
| | CCTGGAGAACTACAGGCCCAACTGGACAGTGCCAGAGTTCA | |
| | CCCTGGACATCTTCAACGCCACCTACCTGAACCTGACAGGAG | |
| | AAATTGACGACCTGGAGTTCAGATCAGAAAAACTTCACAAC | |
| | ACCACCGTGGAGCTTGCCATCCTCATTGACAACATTAACAAC | |
| | ACACTGGTCAACCTGGAATGGCTGAACAGAATTGAAACCTA | |
| | CGTGAAGTGGCCCTGGTATGTGTGGCTGCTGATTGGACTGGT | |
| | GGTGGTGTTCTGCATCCCACTGCTGCTGTTCTGCTGCTTCAG | |
| | CACCGGCTGCTGTGGATGCATCGGCTGCTTGGGCAGCTGCTG | |
| | CCACAGCATCTGCAGCAGGAGGCAGTTTGAGAACTACGAAC | |
| | CAATTGAAAAAGTGCACGTCCAC | |
| 99 | ATGGACTACAAAGATGATGATGACAAGGACTACAAAGACGA | Flag-S(S3) |
| | CGACGACAAAGACTACAAGGACGATGACGACAAGACCACCA | |
| | ACAACGAGTGCATCCAGGTGAACGTGACCCAGCTGGCAGGC | |
| | AACGAGAACCTCATCAGAGACTTCCTCTTCTCCAACTTCAAG | |
| | GAGGAGGGCTCAGTGGTGGTCGGCGGCTACTACCCAACAGA | |
| | GGTGTGGTACAACTGCTCAAGGACCGCCAGAACCACAGCCT | |
| | TCCAGTACTTCAACAACATCCACGCCTTCTACTTCGTGATGG | |
| | AGGCCATGGAGAACTCCACCGGGAACGCCAGGGGCAAGCC | |
| | ACTACTCTTCCACGTGCACGGAGAGCCAGTGAGCGTGATCAT | |
| | CTCAGCCTACAGGGACGACGTGCAGCAGCGCCCCCTGCTGA | |
| | AGCATGGACTGGTGTGCATCACCAAGAACAGGCACATCAAC | |
| | TACGAGCAGTTCACCAGCAACCAGTGGAACAGCACCTGCAC | |
| | CGGCGCAGACAGGAAGATCCCCTTCTCAGTGATCCCAACAG | |
| | ACAACGGAACCAAAATCTACGGCCTGGAGTGGAACGACGAC | |
| | TTCGTGACCGCCTACATCAGCGGCAGGTCCTACCATCTCAAC | |
| | ATCAACACCAACTGGTTCAACAACGTCACCCTCCTCTACAGC | |
| | AGGTCATCCACAGCCACCTGGGAGTACTCAGCTGCCTATGCA | |
| | TACCAGGGAGTCTCCAACTTCACATACTACAAACTCAACAAC | |
| | ACCAACGGCCTCAAGACCTACGAGCTGTGTGAGGACTACGA | |
| | GCACTGCACCGGCTACGCAACAAACGTCTTCGCCCCAACCT | |
| | CCGGAGGCTACATCCCAGACGGCTTCTCCTTCAACAACTGGT | |
| | TCCTCCTCACAAACAGCTCCACCTTCGTGTCAGGAAGGTTCG | |
| | TGACCAACCAGCCCCTGCTCATCAACTGCCTCTGGCCCGTCC | |
| | CCTCCTTCGGAGTGGCCGCCCAGGAGTTCTGCTTCGAGGGA | |
| | GCCCAGTTCTCCCAGTGCAACGGAGTCTCCCTCAACAACAC | |
| | CGTGGACGTCATCAGATTCAACCTCAACTTCACAGCAGACGT | |
| | CCAGAGCGGCATGGGAGCCACCGTGTTCAGCCTGAACACCA | |
| | CAGGAGGAGTGATCCTGGAGATCTCCTGCTACTCAGACACAG | |
| | TGTCAGAGTCCTCCTCCTACAGCTACGGAGAGATCCCATTCG | |
| | GCATCACAGACGGCCCCAGATACTGCTACGTGCTGTACAACG | |
| | GCACAGCCCTGAAGTACCTGGGCACCCTCCCCCCATCAGTGA | |
| | AGGAGATCGCCATCAGCAAGTGGGGCCACTTCTACATCAACG | |
| | GCTACAACTTCTTCTCCACCTTCCCCATCGGCTGCATCAGCTT | |
| | CAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCCTGGTACGTCTGGCTCCTCATCGGCC | |
| | TGGTGGTGGTCTTCTGCATCCCACTGCTGCTGTTCTGCTGCTT | |
| | CTCCACCGGCTGCTGTGGATGCATCGGCTGCCTGGGCTCATG | |
| | CTGCCACTCAATCTGCTCAAGGAGGCAGTTTGAAAACTACGA | |
| | GCCAATAGAAAAAGTCCACGTCCAC | |
| 100 | ATGGACTATAAAGACGACGATGACAAAGATTATAAAGATGAT | Flag-S(S6) |
| | GACGACAAGGACTACAAGGATGATGATGACAAGACCACAAA | |
| | TAACGAGTGCATTCAGGTCAACGTCACCCAGCTGGCCGGTAA | |
| | CGAGAACCTAATTAGAGACTTCCTATTCTCGAACTTTAAAGA | |
| | GGAAGGCTCTGTGGTGGTCGGAGGTTACTACCCCACAGAAG | |
| | TGTGGTACAATTGCTCACGTACAGCCAGGACCACTGCCTTCC | |
| | AGTACTTCAACAACATTCATGCCTTCTACTTTGTCATGGAAGC | |
| | CATGGAGAACTCCACTGGGAATGCCAGAGGAAAGCCTCTCC | |
| | TCTTCCATGTCCATGGAGAGCCTGTCTCTGTGATTATCTCAGC | |
| | ATATAGGGATGATGTGCAGCAGCGGCCGCTGCTTAAGCATGG | |
| | CCTAGTGTGCATTACTAAGAACCGACATATCAATTATGAGCAG | |
| | TTCACCTCCAACCAGTGGAACTCCACATGCACTGGTGCTGAT | |
| | AGGAAGATCCCGTTCAGCGTTATCCCCACCGATAATGGCACA | |
| | AAGATTTATGGCCTAGAATGGAACGATGATTTTGTTACTGCCT | |
| | ACATATCAGGAAGAAGTTACCACTTAAACATTAACACCAATT | |
| | GGTTCAATAATGTTACACTTCTGTACTCTCGCAGCAGTACGGC | |
| | CACTTGGGAGTATTCGGCTGCATATGCCTACCAAGGTGTAAG | |
| | CAACTTCACCTACTACAAGCTGAACAATACGAACGGTCTGAA | |
| | GACTTATGAGCTGTGCGAAGACTACGAGCACTGTACGGGCTA | |
| | TGCGACAAATGTCTTCGCCCCGACGAGCGGCGGGTACATACC | |
| | GGATGGCTTCTCCTTCAACAACTGGTTCCTCCTTACCAATAGC | |
| | TCCACTTTCGTATCAGGAAGATTTGTTACGAACCAACCCCTTC | |
| | TCATTAACTGTCTGTGGCCAGTGCCCTCCTTCGGAGTAGCTG | |
| | CTCAAGAGTTCTGTTTCGAGGGTGCACAGTTCAGCCAGTGTA | |
| | ATGGAGTGTCGCTGAACAACACTGTGGACGTGATCAGGTTTA | |
| | ATTTGAACTTCACAGCTGATGTTCAGTCCGGCATGGGCGCGA | |
| | CTGTGTTCAGCCTAAACACCACGGGTGGCGTCATCTTGGAGA | |
| | TTAGTTGTTACTCTGACACTGTGTCAGAGAGCAGCAGTTACT | |
| | CCTACGGAGAAATTCCTTTCGGCATCACAGACGGTCCCCGGT | |
| | ACTGCTATGTGCTGTACAACGGAACTGCTTTGAAGTACCTGG | |
| | GGACATTGCCACCTTCTGTGAAGGAAATAGCCATCTCTAAGT | |
| | GGGGTCACTTTTACATTAACGGCTATAATTTCTTTTCCACTTTC | |
| | CCAATTGGATGCATTAGCTTCAACCTGACAACAGGTGTGTCT | |
| | GGAGCCTTCTGGACCATCGCCTATACCTCTTACACAGAGGCT | |
| | CTAGTACAGGTGGAGAACACAGCTATAAAGAACGTGACGTA | |
| | CTGTAACAGTCACATAAACAATATCAAGTGTTCTCAGTTGAC | |
| | TGCGAACTTAAACAATGGGTTTTATCCAGTGGCGAGCTCGGA | |
| | GGTGGGGTTTGTAAACAAATCTGTGGTGCTGTTGCCCTCCTT | |
| | CTTCACGTACACTGCAGTGAACATCACCATTGATTTGGGGAT | |
| | GAAACTGTCCGGCTACGGGCAGCCTATAGCATCTACACTGAG | |
| | CAATATCACACTGCCCATGCAGGATAACAATACAGATGTGTAC | |
| | TGTATCCGCTCAAACCAGTTCTCTGTATACGTGCACAGTACAT | |
| | GCAAGAGCTCGCTATGGGACAACATTTTCAACCAGGATTGTA | |
| | CTGATGTGCTTGAAGCAACTGCAGTGATCAAAACAGGCACAT | |
| | GCCCGTTCAGCTTTGATAAGCTCAACAACTACCTAACGTTCA | |
| | ACAAGTTCTGCTTGAGCCTGTCTCCAGTAGGCGCCAATTGCA | |
| | AGTTTGACGTTGCAGCGCGAACACGGACAAACGAACAGGTA | |
| | GTGCGGTCGCTCTATGTTATCTACGAGGAGGGGGACAACATA | |
| | GTCGGGGTTCCATCCGACAACTCAGGTTTGCACGACCTGAGT | |
| | GTGCTCCATTTGGACTCATGCACGGATTATAACATCTACGGGC | |
| | GCACAGGTGTGGGGATAATACGAAGAACAAACTCTACGCTAT | |
| | TGAGCGGGCTCTACTACACCTCATTGAGTGGGGACCTGCTAG | |
| | GGTTCAAGAACGTATCTGACGGTGTGATCTATAGCGTCACAC | |
| | CATGTGACGTATCAGCCCAAGCTGCTGTGATTGACGGGGCGA | |
| | TTGTGGGGGCTATGACTTCAATTAACAGCGAGCTCCTAGGCC | |
| | TGACCCACTGGACTACCACCCCAAACTTCTACTACTACAGCA | |
| | TTTATAACTATACCAGTGAGCGCACCAGGGACACTGCCATTG | |
| | ACAGCAATGACGTCGACTGCGAGCCTGTTATTACCTACAGCA | |
| | ACATCGGTGTTTGTAAGAATGGAGCTCTAGTCTTCATAAACG | |
| | TAACGCACTCTGATGGCGATGTTCAACCAATTTCCACTGGGA | |
| | ACGTAACCATACCCACCAACTTTACTATTTCCGTCCAGGTGGA | |
| | GTACATGCAAGTATATACCACGCCAGTGTCCATCGACTGCGCT | |
| | CGGTATGTGTGCAACGGTAACCCACGCTGCAATAAGCTGCTA | |
| | ACGCAGTACGTCAGCGCCTGCCAGACAATAGAGCAGGCATT | |
| | GGCAATGGGTGCAAGGCTTGAAAACATGGAGGTGGACTCCA | |
| | TGTTGTTCGTGTCTGAAAACGCTCTTAAACTAGCATCCGTGG | |
| | AGGCATTCAACAGTACTGAGAACTTGGACTCTATCTATAAGG | |
| | AGTGGCCCTCCATTGGGGGCAGCTGGCTTGGAGGTCTAAAA | |
| | GACATCCTGCCCAGCCACAACTCCAAGAGGAAGTACGGGTC | |
| | CGCTATAGAGGACCTCCTCTTTGACAAGGTTGTTACTTCTGGT | |
| | CTTGGCACAGTGGACGAAGACTACAAGAGGTGCACAGGAGG | |
| | CTATGATATAGCTGACCTGGTGTGTGCTCAATACTACAACGGT | |
| | ATAATGGTTCTCCCAGGTGTGGCCAACGCTGACAAGATGACA | |
| | ATGTACACAGCCTCTTTAGCTGGAGGCATTACCCTGGGAGCC | |
| | CTTGGGGGTGGCGCAGTGGCAATTCCATTTGCCGTTGCGGTG | |
| | CAGGCCCGACTAAACTATGTCGCACTTCAAACAGATGTGCTC | |
| | AACAAGAACCAACAAATACTGGCCAACGCTTTCAACCAGGC | |
| | CATTGGTAACATTACGCAGGCATTTGGCAAGGTGAATGACGC | |
| | CATCCACCAGACCAGCCAGGGACTTGCCACAGTGGCCAAGG | |
| | CCTTGGCAAAGGTGCAGGATGTCGTGAACACACAGGGTCAG | |
| | GCCCTCTCTCATTTGACAGTGCAGCTTCAGAATAACTTCCAA | |
| | GCAATCAGTTCAAGCATCAGCGACATCTACAACCGGCTGGAC | |
| | CCCCCATCTGCAGATGCGCAGGTGGACAGGCTAATCACTGGA | |
| | CGCTTGACGGCACTAAATGCCTTTGTCAGCCAAACTCTGACC | |
| | CGGCAAGCAGAGGTGCGGGCCAGTAGACAACTGGCCAAAG | |
| | ACAAGGTCAACGAGTGCGTCAGGTCCCAGTCCCAGCGTTTT | |
| | GGATTCTGTGGGAACGGGACGCACCTGTTCTCATTAGCCAAT | |
| | GCTGCACCCAATGGCATGATCTTTTTCCATACTGTTCTACTTC | |
| | CTACTGCCTATGAAACCGTGACCGCTTGGAGCGGCATCTGCG | |
| | CATCTGATGGCGATAGGACCTTCGGGCTGGTCGTTAAGGATG | |
| | TCCAGCTAACGCTGTTCCGGAACTTGGATGACAAGTTCTACC | |
| | TGACCCCCAGGACCATGTACCAGCCGAGAGTGGCAACGAGT | |
| | TCTGACTTCGTGCAAATTGAGGGCTGTGACGTCCTGTTTGTT | |
| | AATGCAACAGTGATCGATCTGCCCAGTATCATACCAGATTACA | |
| | TAGACATAAACCAGACAGTCCAGGACATACTGGAGAATTACA | |
| | GGCCAAACTGGACCGTACCAGAGTTCACGCTGGACATATTCA | |
| | ACGCTACGTACCTCAATTTGACTGGGGAAATTGATGACTTGG | |
| | AGTTCAGGTCGGAGAAGCTCCACAACACCACTGTGGAGCTG | |
| | GCCATCCTGATTGACAACATCAACAACACTCTGGTGAACCTG | |
| | GAGTGGCTAAATCGCATTGAAACCTATGTCAAGTGGCCTTGG | |
| | TACGTTTGGCTACTGATCGGACTCGTGGTAGTCTTCTGCATAC | |
| | CACTCCTGCTATTTTGCTGCTTCAGCACAGGGTGCTGTGGCT | |
| | GCATTGGATGCCTAGGTTCCTGCTGTCACAGTATCTGCAGCA | |
| | GAAGACAATTCGAGAACTACGAGCCCATAGAAAAGGTCCAC | |
| | GTACAT | |
| 101 | ATGGACTACAAGGATGATGATGACAAGGACTACAAAGACGA | Flag- |
| | CGACGACAAGGACTACAAGGATGACGATGACAAGACCACCA | S_ec(S2) |
| | ACAATGAATGCATCCAGGTGAACGTGACCCAGCTGGCAGGC | |
| | AATGAAAATTTGATCAGAGACTTCCTGTTCAGCAACTTCAAG | |
| | GAGGAGGGCAGTGTAGTGGTGGGAGGCTACTACCCAACAGA | |
| | GGTGTGGTACAACTGCAGCAGAACAGCCAGAACCACAGCCT | |
| | TCCAGTACTTCAACAACATCCACGCCTTCTACTTTGTGATGGA | |
| | GGCCATGGAAAACAGCACAGGAAATGCCAGAGGAAAACCC | |
| | CTGCTCTTCCACGTGCACGGAGAGCCCGTGTCAGTCATCATC | |
| | AGCGCCTACAGAGATGACGTCCAGCAGCGGCCCCTGCTGAA | |
| | GCATGGACTGGTCTGCATCACCAAGAACAGACACATCAACTA | |
| | CGAGCAGTTCACCAGCAACCAGTGGAACAGCACCTGCACAG | |
| | GAGCAGACAGAAAAATCCCCTTCAGCGTCATCCCCACAGAC | |
| | AACGGCACCAAAATCTATGGCCTGGAGTGGAATGATGACTTT | |
| | GTGACAGCCTATATCAGCGGCAGGAGCTACCACCTCAACATC | |
| | AACACCAACTGGTTCAACAACGTCACCCTGCTCTACTCCAGA | |
| | TCCAGCACAGCCACCTGGGAGTACAGCGCCGCCTATGCCTAC | |
| | CAGGGAGTCTCCAACTTCACCTACTACAAACTGAACAACAC | |
| | CAACGGCCTGAAAACCTACGAGCTGTGTGAGGACTACGAGC | |
| | ACTGCACAGGCTATGCCACAAATGTGTTTGCCCCAACCAGCG | |
| | GAGGCTACATCCCAGACGGCTTCTCCTTCAACAACTGGTTCC | |
| | TCCTCACCAACTCCTCCACATTTGTGAGCGGCAGATTTGTGA | |
| | CCAACCAGCCCCTGCTGATCAACTGCCTGTGGCCCGTGCCCA | |
| | GCTTTGGAGTGGCAGCCCAGGAGTTCTGCTTCGAGGGAGCC | |
| | CAGTTCAGCCAGTGCAACGGAGTCAGCCTGAACAACACAGT | |
| | GGACGTGATCAGATTCAACCTGAACTTCACAGCAGACGTGC | |
| | AGAGTGGAATGGGAGCCACCGTCTTCAGCCTGAACACCACA | |
| | GGAGGAGTGATCCTGGAGATCAGCTGCTACAGCGACACAGT | |
| | GAGCGAGAGCAGCAGCTACAGCTACGGAGAGATCCCATTTG | |
| | GCATCACAGATGGCCCCAGGTACTGCTACGTCCTGTACAATG | |
| | GAACAGCCCTGAAATACCTGGGCACCCTCCCACCCAGCGTG | |
| | AAGGAGATCGCCATCAGCAAGTGGGGCCACTTCTACATCAAT | |
| | GGCTACAACTTCTTCAGCACCTTCCCCATCGGCTGCATCTCCT | |
| | TCAACCTGACCACAGGAGTGAGCGGGGCCTTCTGGACAATC | |
| | GCCTACACATCCTACACAGAAGCCCTGGTGCAGGTGGAGAA | |
| | CACAGCCATCAAAAACGTCACCTACTGCAACAGCCACATCA | |
| | ACAACATCAAGTGCAGCCAGCTGACAGCCAACCTGAACAAC | |
| | GGCTTCTACCCAGTGGCCAGCTCAGAGGTGGGCTTCGTGAA | |
| | CAAGAGCGTGGTGCTCCTGCCCAGCTTCTTCACCTACACAGC | |
| | AGTGAACATCACAATTGACCTGGGCATGAAGCTGAGCGGCTA | |
| | CGGCCAGCCAATTGCCAGCACCCTCTCCAACATCACCCTCCC | |
| | CATGCAGGACAATAACACAGATGTGTACTGCATCAGATCCAA | |
| | CCAGTTCTCTGTCTACGTGCACAGCACCTGCAAAAGCAGCCT | |
| | GTGGGACAACATCTTCAACCAGGACTGCACAGATGTCCTGG | |
| | AGGCCACAGCCGTGATCAAAACAGGCACCTGCCCCTTCAGC | |
| | TTTGACAAACTCAACAACTACCTTACATTCAACAAATTCTGC | |
| | CTCTCCCTCAGCCCAGTGGGAGCCAACTGCAAGTTTGATGTG | |
| | GCCGCCAGGACCAGGACAAATGAACAAGTGGTCAGAAGCCT | |
| | CTACGTCATCTACGAGGAGGGAGACAACATCGTGGGGGTCC | |
| | CCAGCGACAACAGCGGCCTGCACGACCTGAGTGTGCTCCAC | |
| | CTGGACAGCTGCACAGACTACAACATCTACGGCAGGACTGG | |
| | GGTGGGCATCATCAGAAGAACCAACAGCACACTGCTGAGTG | |
| | GCCTGTACTACACCAGCCTGAGTGGAGACTTGCTGGGCTTCA | |
| | AGAATGTGTCAGATGGGGTGATCTACAGTGTGACCCCCTGTG | |
| | ACGTGTCTGCCCAGGCTGCAGTCATCGACGGAGCCATCGTGG | |
| | GAGCCATGACCAGCATTAACAGCGAGCTGCTGGGCCTGACC | |
| | CACTGGACCACCACCCCCAACTTCTACTACTACTCCATCTACA | |
| | ACTACACATCAGAAAGAACAAGAGACACAGCCATCGACAGC | |
| | AATGACGTGGACTGTGAGCCAGTCATCACCTACAGCAACATC | |
| | GGAGTGTGCAAGAACGGAGCCCTGGTGTTCATCAACGTGAC | |
| | CCACAGCGACGGAGATGTCCAGCCCATCAGCACAGGAAATG | |
| | TGACCATCCCAACCAACTTCACCATCAGCGTCCAGGTGGAAT | |
| | ACATGCAGGTGTACACCACCCCAGTGTCCATCGACTGTGCCA | |
| | GATACGTGTGCAATGGAAACCCCAGATGCAACAAGCTCCTCA | |
| | CCCAGTACGTGTCAGCCTGCCAGACAATCGAGCAGGCCCTG | |
| | GCCATGGGAGCCAGGCTCGAGAACATGGAAGTGGACAGCAT | |
| | GCTGTTTGTCTCAGAGAATGCCCTGAAACTGGCCAGCGTGG | |
| | AGGCCTTCAACAGCACAGAGAACCTGGACAGCATCTACAAG | |
| | GAGTGGCCATCAATCGGAGGCAGCTGGCTGGGAGGACTTAA | |
| | GGACATCCTGCCAAGCCACAACAGCAAAAGAAAGTACGGCA | |
| | GCGCCATTGAGGACCTGCTGTTTGACAAGGTGGTCACCTCCG | |
| | GCCTGGGCACAGTGGATGAGGACTACAAGAGATGCACCGGC | |
| | GGCTATGACATTGCCGACCTGGTGTGTGCCCAGTACTACAAT | |
| | GGCATCATGGTGCTGCCTGGAGTGGCCAACGCCGACAAAAT | |
| | GACCATGTACACCGCCTCCCTGGCTGGAGGCATCACACTGGG | |
| | AGCCCTGGGGGGAGGAGCAGTGGCCATCCCCTTTGCAGTGG | |
| | CTGTGCAGGCCAGACTCAACTACGTGGCCCTGCAGACAGAC | |
| | GTGCTCAACAAGAACCAGCAGATCCTGGCCAACGCTTTCAA | |
| | CCAGGCTATCGGAAACATCACCCAGGCCTTTGGAAAAGTGA | |
| | ATGATGCCATCCACCAGACCAGCCAGGGCCTGGCCACAGTG | |
| | GCCAAGGCCCTGGCCAAGGTGCAGGACGTGGTCAACACCCA | |
| | GGGCCAGGCCCTCAGTCACCTCACAGTACAGCTCCAGAACA | |
| | ACTTCCAGGCAATCTCCTCCTCCATCAGCGACATCTACAACA | |
| | GGCTGGACCCCCCAAGCGCTGATGCCCAGGTGGACAGACTG | |
| | ATCACAGGAAGACTCACAGCCCTCAACGCATTTGTGTCCCAG | |
| | ACACTGACCAGGCAGGCAGAGGTCAGGGCCAGCAGGCAGC | |
| | TGGCCAAGGACAAGGTGAATGAGTGCGTGAGGAGCCAGAG | |
| | CCAGAGATTTGGCTTCTGCGGAAACGGCACCCACCTGTTCAG | |
| | CCTGGCCAACGCCGCCCCCAACGGCATGATTTTCTTCCACAC | |
| | AGTCCTCCTCCCCACAGCCTACGAAACAGTGACAGCCTGGT | |
| | CAGGCATCTGTGCCAGCGACGGAGACAGAACCTTTGGCCTG | |
| | GTGGTGAAGGATGTGCAGCTCACCCTCTTCAGAAACCTGGAT | |
| | GACAAGTTCTACCTCACCCCAAGAACCATGTACCAGCCCAGA | |
| | GTGGCCACAAGCAGCGACTTTGTGCAGATTGAGGGCTGTGA | |
| | CGTGCTGTTTGTGAATGCAACAGTGATTGACCTCCCAAGCAT | |
| | CATCCCAGATTACATCGACATCAACCAGACAGTGCAGGACAT | |
| | CCTGGAGAACTACAGGCCCAACTGGACAGTGCCAGAGTTCA | |
| | CCCTGGACATCTTCAACGCCACCTACCTGAACCTGACAGGAG | |
| | AAATTGACGACCTGGAGTTCAGATCAGAAAAACTTCACAAC | |
| | ACCACCGTGGAGCTTGCCATCCTCATTGACAACATTAACAAC | |
| | ACACTGGTCAACCTGGAATGGCTGAACAGAATTGAAACCTA | |
| | CGTGAAGTGGCCC | |
| 102 | ATGGACTACAAAGATGATGATGACAAGGACTACAAAGACGA | Flag- |
| | CGACGACAAAGACTACAAGGACGATGACGACAAGACCACCA | S_ec(S3) |
| | ACAACGAGTGCATCCAGGTGAACGTGACCCAGCTGGCAGGC | |
| | AACGAGAACCTCATCAGAGACTTCCTCTTCTCCAACTTCAAG | |
| | GAGGAGGGCTCAGTGGTGGTCGGCGGCTACTACCCAACAGA | |
| | GGTGTGGTACAACTGCTCAAGGACCGCCAGAACCACAGCCT | |
| | TCCAGTACTTCAACAACATCCACGCCTTCTACTTCGTGATGG | |
| | AGGCCATGGAGAACTCCACCGGGAACGCCAGGGGCAAGCC | |
| | ACTACTCTTCCACGTGCACGGAGAGCCAGTGAGCGTGATCAT | |
| | CTCAGCCTACAGGGACGACGTGCAGCAGCGCCCCCTGCTGA | |
| | AGCATGGACTGGTGTGCATCACCAAGAACAGGCACATCAAC | |
| | TACGAGCAGTTCACCAGCAACCAGTGGAACAGCACCTGCAC | |
| | CGGCGCAGACAGGAAGATCCCCTTCTCAGTGATCCCAACAG | |
| | ACAACGGAACCAAAATCTACGGCCTGGAGTGGAACGACGAC | |
| | TTCGTGACCGCCTACATCAGCGGCAGGTCCTACCATCTCAAC | |
| | ATCAACACCAACTGGTTCAACAACGTCACCCTCCTCTACAGC | |
| | AGGTCATCCACAGCCACCTGGGAGTACTCAGCTGCCTATGCA | |
| | TACCAGGGAGTCTCCAACTTCACATACTACAAACTCAACAAC | |
| | ACCAACGGCCTCAAGACCTACGAGCTGTGTGAGGACTACGA | |
| | GCACTGCACCGGCTACGCAACAAACGTCTTCGCCCCAACCT | |
| | CCGGAGGCTACATCCCAGACGGCTTCTCCTTCAACAACTGGT | |
| | TCCTCCTCACAAACAGCTCCACCTTCGTGTCAGGAAGGTTCG | |
| | TGACCAACCAGCCCCTGCTCATCAACTGCCTCTGGCCCGTCC | |
| | CCTCCTTCGGAGTGGCCGCCCAGGAGTTCTGCTTCGAGGGA | |
| | GCCCAGTTCTCCCAGTGCAACGGAGTCTCCCTCAACAACAC | |
| | CGTGGACGTCATCAGATTCAACCTCAACTTCACAGCAGACGT | |
| | CCAGAGCGGCATGGGAGCCACCGTGTTCAGCCTGAACACCA | |
| | CAGGAGGAGTGATCCTGGAGATCTCCTGCTACTCAGACACAG | |
| | TGTCAGAGTCCTCCTCCTACAGCTACGGAGAGATCCCATTCG | |
| | GCATCACAGACGGCCCCAGATACTGCTACGTGCTGTACAACG | |
| | GCACAGCCCTGAAGTACCTGGGCACCCTCCCCCCATCAGTGA | |
| | AGGAGATCGCCATCAGCAAGTGGGGCCACTTCTACATCAACG | |
| | GCTACAACTTCTTCTCCACCTTCCCCATCGGCTGCATCAGCTT | |
| | CAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCC | |
| 103 | ATGGACTATAAAGACGACGATGACAAAGATTATAAAGATGAT | Flag- |
| | GACGACAAGGACTACAAGGATGATGATGACAAGACCACAAA | S_ec(S6) |
| | TAACGAGTGCATTCAGGTCAACGTCACCCAGCTGGCCGGTAA | |
| | CGAGAACCTAATTAGAGACTTCCTATTCTCGAACTTTAAAGA | |
| | GGAAGGCTCTGTGGTGGTCGGAGGTTACTACCCCACAGAAG | |
| | TGTGGTACAATTGCTCACGTACAGCCAGGACCACTGCCTTCC | |
| | AGTACTTCAACAACATTCATGCCTTCTACTTTGTCATGGAAGC | |
| | CATGGAGAACTCCACTGGGAATGCCAGAGGAAAGCCTCTCC | |
| | TCTTCCATGTCCATGGAGAGCCTGTCTCTGTGATTATCTCAGC | |
| | ATATAGGGATGATGTGCAGCAGCGGCCGCTGCTTAAGCATGG | |
| | CCTAGTGTGCATTACTAAGAACCGACATATCAATTATGAGCAG | |
| | TTCACCTCCAACCAGTGGAACTCCACATGCACTGGTGCTGAT | |
| | AGGAAGATCCCGTTCAGCGTTATCCCCACCGATAATGGCACA | |
| | AAGATTTATGGCCTAGAATGGAACGATGATTTTGTTACTGCCT | |
| | ACATATCAGGAAGAAGTTACCACTTAAACATTAACACCAATT | |
| | GGTTCAATAATGTTACACTTCTGTACTCTCGCAGCAGTACGGC | |
| | CACTTGGGAGTATTCGGCTGCATATGCCTACCAAGGTGTAAG | |
| | CAACTTCACCTACTACAAGCTGAACAATACGAACGGTCTGAA | |
| | GACTTATGAGCTGTGCGAAGACTACGAGCACTGTACGGGCTA | |
| | TGCGACAAATGTCTTCGCCCCGACGAGCGGCGGGTACATACC | |
| | GGATGGCTTCTCCTTCAACAACTGGTTCCTCCTTACCAATAGC | |
| | TCCACTTTCGTATCAGGAAGATTTGTTACGAACCAACCCCTTC | |
| | TCATTAACTGTCTGTGGCCAGTGCCCTCCTTCGGAGTAGCTG | |
| | CTCAAGAGTTCTGTTTCGAGGGTGCACAGTTCAGCCAGTGTA | |
| | ATGGAGTGTCGCTGAACAACACTGTGGACGTGATCAGGTTTA | |
| | ATTTGAACTTCACAGCTGATGTTCAGTCCGGCATGGGCGCGA | |
| | CTGTGTTCAGCCTAAACACCACGGGTGGCGTCATCTTGGAGA | |
| | TTAGTTGTTACTCTGACACTGTGTCAGAGAGCAGCAGTTACT | |
| | CCTACGGAGAAATTCCTTTCGGCATCACAGACGGTCCCCGGT | |
| | ACTGCTATGTGCTGTACAACGGAACTGCTTTGAAGTACCTGG | |
| | GGACATTGCCACCTTCTGTGAAGGAAATAGCCATCTCTAAGT | |
| | GGGGTCACTTTTACATTAACGGCTATAATTTCTTTTCCACTTTC | |
| | CCAATTGGATGCATTAGCTTCAACCTGACAACAGGTGTGTCT | |
| | GGAGCCTTCTGGACCATCGCCTATACCTCTTACACAGAGGCT | |
| | CTAGTACAGGTGGAGAACACAGCTATAAAGAACGTGACGTA | |
| | CTGTAACAGTCACATAAACAATATCAAGTGTTCTCAGTTGAC | |
| | TGCGAACTTAAACAATGGGTTTTATCCAGTGGCGAGCTCGGA | |
| | GGTGGGGTTTGTAAACAAATCTGTGGTGCTGTTGCCCTCCTT | |
| | CTTCACGTACACTGCAGTGAACATCACCATTGATTTGGGGAT | |
| | GAAACTGTCCGGCTACGGGCAGCCTATAGCATCTACACTGAG | |
| | CAATATCACACTGCCCATGCAGGATAACAATACAGATGTGTAC | |
| | TGTATCCGCTCAAACCAGTTCTCTGTATACGTGCACAGTACAT | |
| | GCAAGAGCTCGCTATGGGACAACATTTTCAACCAGGATTGTA | |
| | CTGATGTGCTTGAAGCAACTGCAGTGATCAAAACAGGCACAT | |
| | GCCCGTTCAGCTTTGATAAGCTCAACAACTACCTAACGTTCA | |
| | ACAAGTTCTGCTTGAGCCTGTCTCCAGTAGGCGCCAATTGCA | |
| | AGTTTGACGTTGCAGCGCGAACACGGACAAACGAACAGGTA | |
| | GTGCGGTCGCTCTATGTTATCTACGAGGAGGGGGACAACATA | |
| | GTCGGGGTTCCATCCGACAACTCAGGTTTGCACGACCTGAGT | |
| | GTGCTCCATTTGGACTCATGCACGGATTATAACATCTACGGGC | |
| | GCACAGGTGTGGGGATAATACGAAGAACAAACTCTACGCTAT | |
| | TGAGCGGGCTCTACTACACCTCATTGAGTGGGGACCTGCTAG | |
| | GGTTCAAGAACGTATCTGACGGTGTGATCTATAGCGTCACAC | |
| | CATGTGACGTATCAGCCCAAGCTGCTGTGATTGACGGGGCGA | |
| | TTGTGGGGGCTATGACTTCAATTAACAGCGAGCTCCTAGGCC | |
| | TGACCCACTGGACTACCACCCCAAACTTCTACTACTACAGCA | |
| | TTTATAACTATACCAGTGAGCGCACCAGGGACACTGCCATTG | |
| | ACAGCAATGACGTCGACTGCGAGCCTGTTATTACCTACAGCA | |
| | ACATCGGTGTTTGTAAGAATGGAGCTCTAGTCTTCATAAACG | |
| | TAACGCACTCTGATGGCGATGTTCAACCAATTTCCACTGGGA | |
| | ACGTAACCATACCCACCAACTTTACTATTTCCGTCCAGGTGGA | |
| | GTACATGCAAGTATATACCACGCCAGTGTCCATCGACTGCGCT | |
| | CGGTATGTGTGCAACGGTAACCCACGCTGCAATAAGCTGCTA | |
| | ACGCAGTACGTCAGCGCCTGCCAGACAATAGAGCAGGCATT | |
| | GGCAATGGGTGCAAGGCTTGAAAACATGGAGGTGGACTCCA | |
| | TGTTGTTCGTGTCTGAAAACGCTCTTAAACTAGCATCCGTGG | |
| | AGGCATTCAACAGTACTGAGAACTTGGACTCTATCTATAAGG | |
| | AGTGGCCCTCCATTGGGGGCAGCTGGCTTGGAGGTCTAAAA | |
| | GACATCCTGCCCAGCCACAACTCCAAGAGGAAGTACGGGTC | |
| | CGCTATAGAGGACCTCCTCTTTGACAAGGTTGTTACTTCTGGT | |
| | CTTGGCACAGTGGACGAAGACTACAAGAGGTGCACAGGAGG | |
| | CTATGATATAGCTGACCTGGTGTGTGCTCAATACTACAACGGT | |
| | ATAATGGTTCTCCCAGGTGTGGCCAACGCTGACAAGATGACA | |
| | ATGTACACAGCCTCTTTAGCTGGAGGCATTACCCTGGGAGCC | |
| | CTTGGGGGTGGCGCAGTGGCAATTCCATTTGCCGTTGCGGTG | |
| | CAGGCCCGACTAAACTATGTCGCACTTCAAACAGATGTGCTC | |
| | AACAAGAACCAACAAATACTGGCCAACGCTTTCAACCAGGC | |
| | CATTGGTAACATTACGCAGGCATTTGGCAAGGTGAATGACGC | |
| | CATCCACCAGACCAGCCAGGGACTTGCCACAGTGGCCAAGG | |
| | CCTTGGCAAAGGTGCAGGATGTCGTGAACACACAGGGTCAG | |
| | GCCCTCTCTCATTTGACAGTGCAGCTTCAGAATAACTTCCAA | |
| | GCAATCAGTTCAAGCATCAGCGACATCTACAACCGGCTGGAC | |
| | CCCCCATCTGCAGATGCGCAGGTGGACAGGCTAATCACTGGA | |
| | CGCTTGACGGCACTAAATGCCTTTGTCAGCCAAACTCTGACC | |
| | CGGCAAGCAGAGGTGCGGGCCAGTAGACAACTGGCCAAAG | |
| | ACAAGGTCAACGAGTGCGTCAGGTCCCAGTCCCAGCGTTTT | |
| | GGATTCTGTGGGAACGGGACGCACCTGTTCTCATTAGCCAAT | |
| | GCTGCACCCAATGGCATGATCTTTTTCCATACTGTTCTACTTC | |
| | CTACTGCCTATGAAACCGTGACCGCTTGGAGCGGCATCTGCG | |
| | CATCTGATGGCGATAGGACCTTCGGGCTGGTCGTTAAGGATG | |
| | TCCAGCTAACGCTGTTCCGGAACTTGGATGACAAGTTCTACC | |
| | TGACCCCCAGGACCATGTACCAGCCGAGAGTGGCAACGAGT | |
| | TCTGACTTCGTGCAAATTGAGGGCTGTGACGTCCTGTTTGTT | |
| | AATGCAACAGTGATCGATCTGCCCAGTATCATACCAGATTACA | |
| | TAGACATAAACCAGACAGTCCAGGACATACTGGAGAATTACA | |
| | GGCCAAACTGGACCGTACCAGAGTTCACGCTGGACATATTCA | |
| | ACGCTACGTACCTCAATTTGACTGGGGAAATTGATGACTTGG | |
| | AGTTCAGGTCGGAGAAGCTCCACAACACCACTGTGGAGCTG | |
| | GCCATCCTGATTGACAACATCAACAACACTCTGGTGAACCTG | |
| | GAGTGGCTAAATCGCATTGAAACCTATGTCAAGTGGCCT | |
| 104 | ATGGACTACAAGGATGATGATGACAAGGACTACAAAGACGA | Flag-SII(S2) |
| | CGACGACAAGGACTACAAGGATGACGATGACAAGTGTGAGC | |
| | CAGTCATCACCTACAGCAACATCGGAGTGTGCAAGAACGGA | |
| | GCCCTGGTGTTCATCAACGTGACCCACAGCGACGGAGATGTC | |
| | CAGCCCATCAGCACAGGAAATGTGACCATCCCAACCAACTTC | |
| | ACCATCAGCGTCCAGGTGGAATACATGCAGGTGTACACCACC | |
| | CCAGTGTCCATCGACTGTGCCAGATACGTGTGCAATGGAAAC | |
| | CCCAGATGCAACAAGCTCCTCACCCAGTACGTGTCAGCCTGC | |
| | CAGACAATCGAGCAGGCCCTGGCCATGGGAGCCAGGCTCGA | |
| | GAACATGGAAGTGGACAGCATGCTGTTTGTCTCAGAGAATGC | |
| | CCTGAAACTGGCCAGCGTGGAGGCCTTCAACAGCACAGAGA | |
| | ACCTGGACAGCATCTACAAGGAGTGGCCATCAATCGGAGGC | |
| | AGCTGGCTGGGAGGACTTAAGGACATCCTGCCAAGCCACAA | |
| | CAGCAAAAGAAAGTACGGCAGCGCCATTGAGGACCTGCTGT | |
| | TTGACAAGGTGGTCACCTCCGGCCTGGGCACAGTGGATGAG | |
| | GACTACAAGAGATGCACCGGCGGCTATGACATTGCCGACCTG | |
| | GTGTGTGCCCAGTACTACAATGGCATCATGGTGCTGCCTGGA | |
| | GTGGCCAACGCCGACAAAATGACCATGTACACCGCCTCCCTG | |
| | GCTGGAGGCATCACACTGGGAGCCCTGGGGGGAGGAGCAGT | |
| | GGCCATCCCCTTTGCAGTGGCTGTGCAGGCCAGACTCAACTA | |
| | CGTGGCCCTGCAGACAGACGTGCTCAACAAGAACCAGCAGA | |
| | TCCTGGCCAACGCTTTCAACCAGGCTATCGGAAACATCACCC | |
| | AGGCCTTTGGAAAAGTGAATGATGCCATCCACCAGACCAGC | |
| | CAGGGCCTGGCCACAGTGGCCAAGGCCCTGGCCAAGGTGCA | |
| | GGACGTGGTCAACACCCAGGGCCAGGCCCTCAGTCACCTCA | |
| | CAGTACAGCTCCAGAACAACTTCCAGGCAATCTCCTCCTCCA | |
| | TCAGCGACATCTACAACAGGCTGGACCCCCCAAGCGCTGAT | |
| | GCCCAGGTGGACAGACTGATCACAGGAAGACTCACAGCCCT | |
| | CAACGCATTTGTGTCCCAGACACTGACCAGGCAGGCAGAGG | |
| | TCAGGGCCAGCAGGCAGCTGGCCAAGGACAAGGTGAATGA | |
| | GTGCGTGAGGAGCCAGAGCCAGAGATTTGGCTTCTGCGGAA | |
| | ACGGCACCCACCTGTTCAGCCTGGCCAACGCCGCCCCCAAC | |
| | GGCATGATTTTCTTCCACACAGTCCTCCTCCCCACAGCCTAC | |
| | GAAACAGTGACAGCCTGGTCAGGCATCTGTGCCAGCGACGG | |
| | AGACAGAACCTTTGGCCTGGTGGTGAAGGATGTGCAGCTCA | |
| | CCCTCTTCAGAAACCTGGATGACAAGTTCTACCTCACCCCAA | |
| | GAACCATGTACCAGCCCAGAGTGGCCACAAGCAGCGACTTT | |
| | GTGCAGATTGAGGGCTGTGACGTGCTGTTTGTGAATGCAACA | |
| | GTGATTGACCTCCCAAGCATCATCCCAGATTACATCGACATCA | |
| | ACCAGACAGTGCAGGACATCCTGGAGAACTACAGGCCCAAC | |
| | TGGACAGTGCCAGAGTTCACCCTGGACATCTTCAACGCCACC | |
| | TACCTGAACCTGACAGGAGAAATTGACGACCTGGAGTTCAG | |
| | ATCAGAAAAACTTCACAACACCACCGTGGAGCTTGCCATCCT | |
| | CATTGACAACATTAACAACACACTGGTCAACCTGGAATGGCT | |
| | GAACAGAATTGAAACCTACGTGAAGTGGCCCTGGTATGTGTG | |
| | GCTGCTGATTGGACTGGTGGTGGTGTTCTGCATCCCACTGCT | |
| | GCTGTTCTGCTGCTTCAGCACCGGCTGCTGTGGATGCATCGG | |
| | CTGCTTGGGCAGCTGCTGCCACAGCATCTGCAGCAGGAGGC | |
| | AGTTTGAGAACTACGAACCAATTGAAAAAGTGCACGTCCAC | |
| 105 | ATGGACTACAAAGATGATGATGACAAGGACTACAAAGACGA | Flag-SII(S3) |
| | CGACGACAAAGACTACAAGGACGATGACGACAAGTGTGAGC | |
| | CAGTCATCACCTACTCCAACATCGGCGTGTGCAAGAACGGAG | |
| | CCCTGGTGTTCATCAACGTCACCCACTCAGACGGCGACGTCC | |
| | AGCCAATCTCCACAGGAAACGTCACCATCCCCACCAACTTCA | |
| | CCATCAGCGTGCAGGTGGAGTACATGCAGGTCTACACCACCC | |
| | CAGTCTCCATCGACTGTGCCAGGTACGTGTGCAACGGCAACC | |
| | CAAGATGCAACAAACTGCTGACCCAGTACGTGAGCGCCTGC | |
| | CAGACCATCGAGCAGGCCCTGGCCATGGGCGCCAGGCTGGA | |
| | GAACATGGAGGTGGACAGCATGCTCTTTGTGAGCGAGAACG | |
| | CCCTGAAGCTTGCCAGCGTGGAGGCCTTCAACAGCACCGAA | |
| | AACCTGGACTCCATCTACAAAGAGTGGCCCTCCATAGGAGGC | |
| | TCCTGGCTGGGAGGCCTGAAGGACATCCTCCCATCCCACAAC | |
| | AGCAAAAGAAAGTACGGCAGCGCCATCGAAGACCTGCTGTT | |
| | CGACAAGGTGGTCACCTCAGGACTGGGCACAGTGGACGAGG | |
| | ACTACAAGAGGTGCACCGGAGGCTACGACATCGCAGACCTG | |
| | GTCTGTGCCCAGTACTACAACGGCATCATGGTGCTCCCAGGC | |
| | GTGGCCAACGCCGACAAGATGACCATGTACACAGCAAGCCT | |
| | GGCTGGAGGAATCACACTGGGAGCCCTGGGAGGAGGGGCC | |
| | GTGGCCATTCCATTCGCCGTGGCCGTGCAGGCCAGACTGAAC | |
| | TACGTGGCCCTGCAGACAGACGTGCTAAACAAGAACCAGCA | |
| | GATCCTGGCCAACGCCTTCAACCAGGCCATCGGCAACATCAC | |
| | CCAGGCCTTCGGCAAGGTGAACGACGCAATCCACCAGACAT | |
| | CACAGGGCCTGGCAACAGTGGCCAAGGCCCTGGCCAAGGTC | |
| | CAGGACGTGGTGAACACCCAGGGCCAGGCCCTCTCACACCT | |
| | GACAGTCCAGCTGCAGAACAACTTCCAGGCAATCTCCTCCTC | |
| | CATCTCAGACATCTACAACAGACTGGACCCCCCCTCAGCCGA | |
| | CGCCCAGGTGGACAGACTCATCACAGGCAGGCTGACCGCCC | |
| | TCAACGCCTTCGTGTCCCAGACCCTCACCAGGCAGGCCGAG | |
| | GTGAGGGCCAGCAGGCAGCTCGCCAAGGACAAGGTGAACG | |
| | AGTGCGTCAGAAGCCAGAGCCAGAGGTTCGGCTTCTGTGGC | |
| | AACGGCACCCACCTGTTCTCCCTGGCCAACGCAGCCCCCAA | |
| | CGGCATGATCTTCTTCCACACAGTCCTCCTCCCAACAGCATAT | |
| | GAGACAGTCACCGCCTGGTCAGGAATCTGTGCCTCAGACGG | |
| | GGACAGAACCTTCGGCCTGGTGGTCAAGGACGTGCAGCTGA | |
| | CACTCTTCAGAAACCTGGACGACAAATTCTACCTGACCCCCA | |
| | GGACCATGTACCAGCCAAGGGTGGCCACCTCCTCAGACTTCG | |
| | TGCAGATCGAGGGCTGTGACGTGCTCTTCGTGAACGCCACC | |
| | GTCATCGACCTCCCATCCATCATCCCAGACTACATCGACATCA | |
| | ACCAGACAGTGCAGGACATCCTGGAGAACTACCGCCCCAAC | |
| | TGGACCGTGCCAGAGTTCACCCTAGACATATTCAACGCCACC | |
| | TACCTGAACCTGACAGGAGAAATTGACGACCTGGAGTTCAG | |
| | ATCAGAAAAGCTACACAACACCACCGTGGAGTTAGCCATCCT | |
| | CATAGACAACATTAACAACACCCTCGTCAACCTGGAGTGGCT | |
| | CAACAGGATTGAAACCTACGTGAAGTGGCCCTGGTACGTCTG | |
| | GCTCCTCATCGGCCTGGTGGTGGTCTTCTGCATCCCACTGCT | |
| | GCTGTTCTGCTGCTTCTCCACCGGCTGCTGTGGATGCATCGG | |
| | CTGCCTGGGCTCATGCTGCCACTCAATCTGCTCAAGGAGGCA | |
| | GTTTGAAAACTACGAGCCAATAGAAAAAGTCCACGTCCAC | |
| 106 | ATGGACTATAAAGACGACGATGACAAAGATTATAAAGATGAT | Flag-SII(S6) |
| | GACGACAAGGACTACAAGGATGATGATGACAAGTGCGAGCC | |
| | TGTTATTACCTACAGCAACATCGGTGTTTGTAAGAATGGAGCT | |
| | CTAGTCTTCATAAACGTAACGCACTCTGATGGCGATGTTCAA | |
| | CCAATTTCCACTGGGAACGTAACCATACCCACCAACTTTACT | |
| | ATTTCCGTCCAGGTGGAGTACATGCAAGTATATACCACGCCA | |
| | GTGTCCATCGACTGCGCTCGGTATGTGTGCAACGGTAACCCA | |
| | CGCTGCAATAAGCTGCTAACGCAGTACGTCAGCGCCTGCCAG | |
| | ACAATAGAGCAGGCATTGGCAATGGGTGCAAGGCTTGAAAA | |
| | CATGGAGGTGGACTCCATGTTGTTCGTGTCTGAAAACGCTCT | |
| | TAAACTAGCATCCGTGGAGGCATTCAACAGTACTGAGAACTT | |
| | GGACTCTATCTATAAGGAGTGGCCCTCCATTGGGGGCAGCTG | |
| | GCTTGGAGGTCTAAAAGACATCCTGCCCAGCCACAACTCCA | |
| | AGAGGAAGTACGGGTCCGCTATAGAGGACCTCCTCTTTGACA | |
| | AGGTTGTTACTTCTGGTCTTGGCACAGTGGACGAAGACTACA | |
| | AGAGGTGCACAGGAGGCTATGATATAGCTGACCTGGTGTGTG | |
| | CTCAATACTACAACGGTATAATGGTTCTCCCAGGTGTGGCCA | |
| | ACGCTGACAAGATGACAATGTACACAGCCTCTTTAGCTGGAG | |
| | GCATTACCCTGGGAGCCCTTGGGGGTGGCGCAGTGGCAATTC | |
| | CATTTGCCGTTGCGGTGCAGGCCCGACTAAACTATGTCGCAC | |
| | TTCAAACAGATGTGCTCAACAAGAACCAACAAATACTGGCC | |
| | AACGCTTTCAACCAGGCCATTGGTAACATTACGCAGGCATTT | |
| | GGCAAGGTGAATGACGCCATCCACCAGACCAGCCAGGGACT | |
| | TGCCACAGTGGCCAAGGCCTTGGCAAAGGTGCAGGATGTCG | |
| | TGAACACACAGGGTCAGGCCCTCTCTCATTTGACAGTGCAGC | |
| | TTCAGAATAACTTCCAAGCAATCAGTTCAAGCATCAGCGACA | |
| | TCTACAACCGGCTGGACCCCCCATCTGCAGATGCGCAGGTGG | |
| | ACAGGCTAATCACTGGACGCTTGACGGCACTAAATGCCTTTG | |
| | TCAGCCAAACTCTGACCCGGCAAGCAGAGGTGCGGGCCAGT | |
| | AGACAACTGGCCAAAGACAAGGTCAACGAGTGCGTCAGGTC | |
| | CCAGTCCCAGCGTTTTGGATTCTGTGGGAACGGGACGCACCT | |
| | GTTCTCATTAGCCAATGCTGCACCCAATGGCATGATCTTTTTC | |
| | CATACTGTTCTACTTCCTACTGCCTATGAAACCGTGACCGCTT | |
| | GGAGCGGCATCTGCGCATCTGATGGCGATAGGACCTTCGGGC | |
| | TGGTCGTTAAGGATGTCCAGCTAACGCTGTTCCGGAACTTGG | |
| | ATGACAAGTTCTACCTGACCCCCAGGACCATGTACCAGCCGA | |
| | GAGTGGCAACGAGTTCTGACTTCGTGCAAATTGAGGGCTGT | |
| | GACGTCCTGTTTGTTAATGCAACAGTGATCGATCTGCCCAGT | |
| | ATCATACCAGATTACATAGACATAAACCAGACAGTCCAGGAC | |
| | ATACTGGAGAATTACAGGCCAAACTGGACCGTACCAGAGTTC | |
| | ACGCTGGACATATTCAACGCTACGTACCTCAATTTGACTGGG | |
| | GAAATTGATGACTTGGAGTTCAGGTCGGAGAAGCTCCACAA | |
| | CACCACTGTGGAGCTGGCCATCCTGATTGACAACATCAACAA | |
| | CACTCTGGTGAACCTGGAGTGGCTAAATCGCATTGAAACCTA | |
| | TGTCAAGTGGCCTTGGTACGTTTGGCTACTGATCGGACTCGT | |
| | GGTAGTCTTCTGCATACCACTCCTGCTATTTTGCTGCTTCAGC | |
| | ACAGGGTGCTGTGGCTGCATTGGATGCCTAGGTTCCTGCTGT | |
| | CACAGTATCTGCAGCAGAAGACAATTCGAGAACTACGAGCC | |
| | CATAGAAAAGGTCCACGTACAT | |
| 107 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag-S(S2) |
| | AAGGATGATGATGACAAGGACTACAAAGACGACGACGACAA | |
| | GGACTACAAGGATGACGATGACAAGACCACCAACAATGAAT | |
| | GCATCCAGGTGAACGTGACCCAGCTGGCAGGCAATGAAAAT | |
| | TTGATCAGAGACTTCCTGTTCAGCAACTTCAAGGAGGAGGG | |
| | CAGTGTAGTGGTGGGAGGCTACTACCCAACAGAGGTGTGGT | |
| | ACAACTGCAGCAGAACAGCCAGAACCACAGCCTTCCAGTAC | |
| | TTCAACAACATCCACGCCTTCTACTTTGTGATGGAGGCCATG | |
| | GAAAACAGCACAGGAAATGCCAGAGGAAAACCCCTGCTCTT | |
| | CCACGTGCACGGAGAGCCCGTGTCAGTCATCATCAGCGCCTA | |
| | CAGAGATGACGTCCAGCAGCGGCCCCTGCTGAAGCATGGAC | |
| | TGGTCTGCATCACCAAGAACAGACACATCAACTACGAGCAG | |
| | TTCACCAGCAACCAGTGGAACAGCACCTGCACAGGAGCAGA | |
| | CAGAAAAATCCCCTTCAGCGTCATCCCCACAGACAACGGCA | |
| | CCAAAATCTATGGCCTGGAGTGGAATGATGACTTTGTGACAG | |
| | CCTATATCAGCGGCAGGAGCTACCACCTCAACATCAACACCA | |
| | ACTGGTTCAACAACGTCACCCTGCTCTACTCCAGATCCAGCA | |
| | CAGCCACCTGGGAGTACAGCGCCGCCTATGCCTACCAGGGA | |
| | GTCTCCAACTTCACCTACTACAAACTGAACAACACCAACGGC | |
| | CTGAAAACCTACGAGCTGTGTGAGGACTACGAGCACTGCAC | |
| | AGGCTATGCCACAAATGTGTTTGCCCCAACCAGCGGAGGCTA | |
| | CATCCCAGACGGCTTCTCCTTCAACAACTGGTTCCTCCTCAC | |
| | CAACTCCTCCACATTTGTGAGCGGCAGATTTGTGACCAACCA | |
| | GCCCCTGCTGATCAACTGCCTGTGGCCCGTGCCCAGCTTTGG | |
| | AGTGGCAGCCCAGGAGTTCTGCTTCGAGGGAGCCCAGTTCA | |
| | GCCAGTGCAACGGAGTCAGCCTGAACAACACAGTGGACGTG | |
| | ATCAGATTCAACCTGAACTTCACAGCAGACGTGCAGAGTGG | |
| | AATGGGAGCCACCGTCTTCAGCCTGAACACCACAGGAGGAG | |
| | TGATCCTGGAGATCAGCTGCTACAGCGACACAGTGAGCGAG | |
| | AGCAGCAGCTACAGCTACGGAGAGATCCCATTTGGCATCACA | |
| | GATGGCCCCAGGTACTGCTACGTCCTGTACAATGGAACAGCC | |
| | CTGAAATACCTGGGCACCCTCCCACCCAGCGTGAAGGAGAT | |
| | CGCCATCAGCAAGTGGGGCCACTTCTACATCAATGGCTACAA | |
| | CTTCTTCAGCACCTTCCCCATCGGCTGCATCTCCTTCAACCTG | |
| | ACCACAGGAGTGAGCGGGGCCTTCTGGACAATCGCCTACAC | |
| | ATCCTACACAGAAGCCCTGGTGCAGGTGGAGAACACAGCCA | |
| | TCAAAAACGTCACCTACTGCAACAGCCACATCAACAACATC | |
| | AAGTGCAGCCAGCTGACAGCCAACCTGAACAACGGCTTCTA | |
| | CCCAGTGGCCAGCTCAGAGGTGGGCTTCGTGAACAAGAGCG | |
| | TGGTGCTCCTGCCCAGCTTCTTCACCTACACAGCAGTGAACA | |
| | TCACAATTGACCTGGGCATGAAGCTGAGCGGCTACGGCCAG | |
| | CCAATTGCCAGCACCCTCTCCAACATCACCCTCCCCATGCAG | |
| | GACAATAACACAGATGTGTACTGCATCAGATCCAACCAGTTC | |
| | TCTGTCTACGTGCACAGCACCTGCAAAAGCAGCCTGTGGGA | |
| | CAACATCTTCAACCAGGACTGCACAGATGTCCTGGAGGCCA | |
| | CAGCCGTGATCAAAACAGGCACCTGCCCCTTCAGCTTTGACA | |
| | AACTCAACAACTACCTTACATTCAACAAATTCTGCCTCTCCCT | |
| | CAGCCCAGTGGGAGCCAACTGCAAGTTTGATGTGGCCGCCA | |
| | GGACCAGGACAAATGAACAAGTGGTCAGAAGCCTCTACGTC | |
| | ATCTACGAGGAGGGAGACAACATCGTGGGGGTCCCCAGCGA | |
| | CAACAGCGGCCTGCACGACCTGAGTGTGCTCCACCTGGACA | |
| | GCTGCACAGACTACAACATCTACGGCAGGACTGGGGTGGGC | |
| | ATCATCAGAAGAACCAACAGCACACTGCTGAGTGGCCTGTA | |
| | CTACACCAGCCTGAGTGGAGACTTGCTGGGCTTCAAGAATGT | |
| | GTCAGATGGGGTGATCTACAGTGTGACCCCCTGTGACGTGTC | |
| | TGCCCAGGCTGCAGTCATCGACGGAGCCATCGTGGGAGCCAT | |
| | GACCAGCATTAACAGCGAGCTGCTGGGCCTGACCCACTGGA | |
| | CCACCACCCCCAACTTCTACTACTACTCCATCTACAACTACAC | |
| | ATCAGAAAGAACAAGAGACACAGCCATCGACAGCAATGACG | |
| | TGGACTGTGAGCCAGTCATCACCTACAGCAACATCGGAGTGT | |
| | GCAAGAACGGAGCCCTGGTGTTCATCAACGTGACCCACAGC | |
| | GACGGAGATGTCCAGCCCATCAGCACAGGAAATGTGACCAT | |
| | CCCAACCAACTTCACCATCAGCGTCCAGGTGGAATACATGCA | |
| | GGTGTACACCACCCCAGTGTCCATCGACTGTGCCAGATACGT | |
| | GTGCAATGGAAACCCCAGATGCAACAAGCTCCTCACCCAGT | |
| | ACGTGTCAGCCTGCCAGACAATCGAGCAGGCCCTGGCCATG | |
| | GGAGCCAGGCTCGAGAACATGGAAGTGGACAGCATGCTGTT | |
| | TGTCTCAGAGAATGCCCTGAAACTGGCCAGCGTGGAGGCCT | |
| | TCAACAGCACAGAGAACCTGGACAGCATCTACAAGGAGTGG | |
| | CCATCAATCGGAGGCAGCTGGCTGGGAGGACTTAAGGACAT | |
| | CCTGCCAAGCCACAACAGCAAAAGAAAGTACGGCAGCGCC | |
| | ATTGAGGACCTGCTGTTTGACAAGGTGGTCACCTCCGGCCTG | |
| | GGCACAGTGGATGAGGACTACAAGAGATGCACCGGCGGCTA | |
| | TGACATTGCCGACCTGGTGTGTGCCCAGTACTACAATGGCAT | |
| | CATGGTGCTGCCTGGAGTGGCCAACGCCGACAAAATGACCA | |
| | TGTACACCGCCTCCCTGGCTGGAGGCATCACACTGGGAGCCC | |
| | TGGGGGGAGGAGCAGTGGCCATCCCCTTTGCAGTGGCTGTG | |
| | CAGGCCAGACTCAACTACGTGGCCCTGCAGACAGACGTGCT | |
| | CAACAAGAACCAGCAGATCCTGGCCAACGCTTTCAACCAGG | |
| | CTATCGGAAACATCACCCAGGCCTTTGGAAAAGTGAATGATG | |
| | CCATCCACCAGACCAGCCAGGGCCTGGCCACAGTGGCCAAG | |
| | GCCCTGGCCAAGGTGCAGGACGTGGTCAACACCCAGGGCCA | |
| | GGCCCTCAGTCACCTCACAGTACAGCTCCAGAACAACTTCC | |
| | AGGCAATCTCCTCCTCCATCAGCGACATCTACAACAGGCTGG | |
| | ACCCCCCAAGCGCTGATGCCCAGGTGGACAGACTGATCACA | |
| | GGAAGACTCACAGCCCTCAACGCATTTGTGTCCCAGACACT | |
| | GACCAGGCAGGCAGAGGTCAGGGCCAGCAGGCAGCTGGCC | |
| | AAGGACAAGGTGAATGAGTGCGTGAGGAGCCAGAGCCAGA | |
| | GATTTGGCTTCTGCGGAAACGGCACCCACCTGTTCAGCCTGG | |
| | CCAACGCCGCCCCCAACGGCATGATTTTCTTCCACACAGTCC | |
| | TCCTCCCCACAGCCTACGAAACAGTGACAGCCTGGTCAGGC | |
| | ATCTGTGCCAGCGACGGAGACAGAACCTTTGGCCTGGTGGT | |
| | GAAGGATGTGCAGCTCACCCTCTTCAGAAACCTGGATGACA | |
| | AGTTCTACCTCACCCCAAGAACCATGTACCAGCCCAGAGTGG | |
| | CCACAAGCAGCGACTTTGTGCAGATTGAGGGCTGTGACGTG | |
| | CTGTTTGTGAATGCAACAGTGATTGACCTCCCAAGCATCATC | |
| | CCAGATTACATCGACATCAACCAGACAGTGCAGGACATCCTG | |
| | GAGAACTACAGGCCCAACTGGACAGTGCCAGAGTTCACCCT | |
| | GGACATCTTCAACGCCACCTACCTGAACCTGACAGGAGAAAT | |
| | TGACGACCTGGAGTTCAGATCAGAAAAACTTCACAACACCA | |
| | CCGTGGAGCTTGCCATCCTCATTGACAACATTAACAACACAC | |
| | TGGTCAACCTGGAATGGCTGAACAGAATTGAAACCTACGTG | |
| | AAGTGGCCCTGGTATGTGTGGCTGCTGATTGGACTGGTGGTG | |
| | GTGTTCTGCATCCCACTGCTGCTGTTCTGCTGCTTCAGCACC | |
| | GGCTGCTGTGGATGCATCGGCTGCTTGGGCAGCTGCTGCCAC | |
| | AGCATCTGCAGCAGGAGGCAGTTTGAGAACTACGAACCAAT | |
| | TGAAAAAGTGCACGTCCAC | |
| 108 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag-S(S3) |
| | CAAAGATGATGATGACAAGGACTACAAAGACGACGACGACA | |
| | AAGACTACAAGGACGATGACGACAAGACCACCAACAACGA | |
| | GTGCATCCAGGTGAACGTGACCCAGCTGGCAGGCAACGAGA | |
| | ACCTCATCAGAGACTTCCTCTTCTCCAACTTCAAGGAGGAGG | |
| | GCTCAGTGGTGGTCGGCGGCTACTACCCAACAGAGGTGTGG | |
| | TACAACTGCTCAAGGACCGCCAGAACCACAGCCTTCCAGTA | |
| | CTTCAACAACATCCACGCCTTCTACTTCGTGATGGAGGCCAT | |
| | GGAGAACTCCACCGGGAACGCCAGGGGCAAGCCACTACTCT | |
| | TCCACGTGCACGGAGAGCCAGTGAGCGTGATCATCTCAGCCT | |
| | ACAGGGACGACGTGCAGCAGCGCCCCCTGCTGAAGCATGGA | |
| | CTGGTGTGCATCACCAAGAACAGGCACATCAACTACGAGCA | |
| | GTTCACCAGCAACCAGTGGAACAGCACCTGCACCGGCGCAG | |
| | ACAGGAAGATCCCCTTCTCAGTGATCCCAACAGACAACGGA | |
| | ACCAAAATCTACGGCCTGGAGTGGAACGACGACTTCGTGAC | |
| | CGCCTACATCAGCGGCAGGTCCTACCATCTCAACATCAACAC | |
| | CAACTGGTTCAACAACGTCACCCTCCTCTACAGCAGGTCATC | |
| | CACAGCCACCTGGGAGTACTCAGCTGCCTATGCATACCAGGG | |
| | AGTCTCCAACTTCACATACTACAAACTCAACAACACCAACGG | |
| | CCTCAAGACCTACGAGCTGTGTGAGGACTACGAGCACTGCA | |
| | CCGGCTACGCAACAAACGTCTTCGCCCCAACCTCCGGAGGC | |
| | TACATCCCAGACGGCTTCTCCTTCAACAACTGGTTCCTCCTC | |
| | ACAAACAGCTCCACCTTCGTGTCAGGAAGGTTCGTGACCAA | |
| | CCAGCCCCTGCTCATCAACTGCCTCTGGCCCGTCCCCTCCTT | |
| | CGGAGTGGCCGCCCAGGAGTTCTGCTTCGAGGGAGCCCAGT | |
| | TCTCCCAGTGCAACGGAGTCTCCCTCAACAACACCGTGGAC | |
| | GTCATCAGATTCAACCTCAACTTCACAGCAGACGTCCAGAGC | |
| | GGCATGGGAGCCACCGTGTTCAGCCTGAACACCACAGGAGG | |
| | AGTGATCCTGGAGATCTCCTGCTACTCAGACACAGTGTCAGA | |
| | GTCCTCCTCCTACAGCTACGGAGAGATCCCATTCGGCATCAC | |
| | AGACGGCCCCAGATACTGCTACGTGCTGTACAACGGCACAG | |
| | CCCTGAAGTACCTGGGCACCCTCCCCCCATCAGTGAAGGAG | |
| | ATCGCCATCAGCAAGTGGGGCCACTTCTACATCAACGGCTAC | |
| | AACTTCTTCTCCACCTTCCCCATCGGCTGCATCAGCTTCAACC | |
| | TGACCACCGGAGTGTCCGGAGCCTTCTGGACCATCGCCTACA | |
| | CATCATACACCGAGGCCCTGGTGCAGGTGGAGAACACAGCC | |
| | ATAAAGAACGTGACCTACTGCAACAGCCACATCAACAACATC | |
| | AAGTGCTCCCAGCTGACAGCCAACCTGAACAACGGCTTCTA | |
| | CCCAGTGGCCTCCAGCGAGGTGGGCTTCGTGAACAAGAGCG | |
| | TGGTCCTACTCCCCTCCTTCTTCACCTACACAGCAGTCAACAT | |
| | CACAATTGACCTGGGCATGAAGCTGTCCGGCTACGGCCAGCC | |
| | AATCGCCAGCACCCTGTCCAACATCACCCTGCCAATGCAGGA | |
| | CAACAACACCGACGTCTACTGCATCAGAAGCAACCAGTTCTC | |
| | CGTGTACGTCCACTCCACCTGCAAGTCCTCCCTCTGGGACAA | |
| | CATCTTCAACCAGGACTGCACAGACGTGCTGGAGGCCACAG | |
| | CTGTGATCAAGACAGGAACCTGCCCTTTCTCATTCGACAAGC | |
| | TCAACAACTACCTGACCTTCAACAAGTTCTGCCTGAGCCTGT | |
| | CCCCAGTGGGAGCCAACTGCAAGTTCGACGTGGCCGCCAGA | |
| | ACCAGGACCAACGAGCAGGTGGTCAGAAGCCTGTACGTCAT | |
| | CTACGAGGAGGGAGACAACATCGTGGGAGTGCCCAGCGACA | |
| | ACTCAGGCCTGCACGACCTGAGCGTGCTGCACCTGGACTCC | |
| | TGCACAGACTACAACATCTACGGCAGGACAGGAGTGGGCAT | |
| | CATCAGGAGGACCAACAGCACACTGCTGTCCGGCCTCTACTA | |
| | CACCTCCCTGTCCGGAGACTTGCTGGGATTCAAGAACGTGTC | |
| | AGACGGAGTCATCTACAGCGTCACCCCATGTGACGTGAGCGC | |
| | CCAGGCAGCAGTGATAGACGGAGCCATCGTGGGAGCCATGA | |
| | CCTCAATCAACTCAGAACTGCTGGGCCTCACCCACTGGACA | |
| | ACAACACCCAACTTCTACTACTACTCCATCTACAACTACACAT | |
| | CAGAAAGAACAAGGGACACAGCAATCGACTCCAACGACGT | |
| | GGACTGTGAGCCAGTCATCACCTACTCCAACATCGGCGTGTG | |
| | CAAGAACGGAGCCCTGGTGTTCATCAACGTCACCCACTCAG | |
| | ACGGCGACGTCCAGCCAATCTCCACAGGAAACGTCACCATC | |
| | CCCACCAACTTCACCATCAGCGTGCAGGTGGAGTACATGCAG | |
| | GTCTACACCACCCCAGTCTCCATCGACTGTGCCAGGTACGTG | |
| | TGCAACGGCAACCCAAGATGCAACAAACTGCTGACCCAGTA | |
| | CGTGAGCGCCTGCCAGACCATCGAGCAGGCCCTGGCCATGG | |
| | GCGCCAGGCTGGAGAACATGGAGGTGGACAGCATGCTCTTT | |
| | GTGAGCGAGAACGCCCTGAAGCTTGCCAGCGTGGAGGCCTT | |
| | CAACAGCACCGAAAACCTGGACTCCATCTACAAAGAGTGGC | |
| | CCTCCATAGGAGGCTCCTGGCTGGGAGGCCTGAAGGACATCC | |
| | TCCCATCCCACAACAGCAAAAGAAAGTACGGCAGCGCCATC | |
| | GAAGACCTGCTGTTCGACAAGGTGGTCACCTCAGGACTGGG | |
| | CACAGTGGACGAGGACTACAAGAGGTGCACCGGAGGCTACG | |
| | ACATCGCAGACCTGGTCTGTGCCCAGTACTACAACGGCATCA | |
| | TGGTGCTCCCAGGCGTGGCCAACGCCGACAAGATGACCATG | |
| | TACACAGCAAGCCTGGCTGGAGGAATCACACTGGGAGCCCT | |
| | GGGAGGAGGGGCCGTGGCCATTCCATTCGCCGTGGCCGTGC | |
| | AGGCCAGACTGAACTACGTGGCCCTGCAGACAGACGTGCTA | |
| | AACAAGAACCAGCAGATCCTGGCCAACGCCTTCAACCAGGC | |
| | CATCGGCAACATCACCCAGGCCTTCGGCAAGGTGAACGACG | |
| | CAATCCACCAGACATCACAGGGCCTGGCAACAGTGGCCAAG | |
| | GCCCTGGCCAAGGTCCAGGACGTGGTGAACACCCAGGGCCA | |
| | GGCCCTCTCACACCTGACAGTCCAGCTGCAGAACAACTTCC | |
| | AGGCAATCTCCTCCTCCATCTCAGACATCTACAACAGACTGG | |
| | ACCCCCCCTCAGCCGACGCCCAGGTGGACAGACTCATCACA | |
| | GGCAGGCTGACCGCCCTCAACGCCTTCGTGTCCCAGACCCTC | |
| | ACCAGGCAGGCCGAGGTGAGGGCCAGCAGGCAGCTCGCCA | |
| | AGGACAAGGTGAACGAGTGCGTCAGAAGCCAGAGCCAGAG | |
| | GTTCGGCTTCTGTGGCAACGGCACCCACCTGTTCTCCCTGGC | |
| | CAACGCAGCCCCCAACGGCATGATCTTCTTCCACACAGTCCT | |
| | CCTCCCAACAGCATATGAGACAGTCACCGCCTGGTCAGGAAT | |
| | CTGTGCCTCAGACGGGGACAGAACCTTCGGCCTGGTGGTCA | |
| | AGGACGTGCAGCTGACACTCTTCAGAAACCTGGACGACAAA | |
| | TTCTACCTGACCCCCAGGACCATGTACCAGCCAAGGGTGGCC | |
| | ACCTCCTCAGACTTCGTGCAGATCGAGGGCTGTGACGTGCTC | |
| | TTCGTGAACGCCACCGTCATCGACCTCCCATCCATCATCCCA | |
| | GACTACATCGACATCAACCAGACAGTGCAGGACATCCTGGA | |
| | GAACTACCGCCCCAACTGGACCGTGCCAGAGTTCACCCTAG | |
| | ACATATTCAACGCCACCTACCTGAACCTGACAGGAGAAATTG | |
| | ACGACCTGGAGTTCAGATCAGAAAAGCTACACAACACCACC | |
| | GTGGAGTTAGCCATCCTCATAGACAACATTAACAACACCCTC | |
| | GTCAACCTGGAGTGGCTCAACAGGATTGAAACCTACGTGAA | |
| | GTGGCCCTGGTACGTCTGGCTCCTCATCGGCCTGGTGGTGGT | |
| | CTTCTGCATCCCACTGCTGCTGTTCTGCTGCTTCTCCACCGGC | |
| | TGCTGTGGATGCATCGGCTGCCTGGGCTCATGCTGCCACTCA | |
| | ATCTGCTCAAGGAGGCAGTTTGAAAACTACGAGCCAATAGA | |
| | AAAAGTCCACGTCCAC | |
| 109 | ATGCGTTTTGTAATGTCACCTACTGTACTACTACTACTACTCG | MHCIsp- |
| | GAGCACTAGCAGCACCTCAGACTTGGGCCGGATCAGACTATA | Flag-S(S6) |
| | AAGACGACGATGACAAAGATTATAAAGATGATGACGACAAG | |
| | GACTACAAGGATGATGATGACAAGACCACAAATAACGAGTG | |
| | CATTCAGGTCAACGTCACCCAGCTGGCCGGTAACGAGAACC | |
| | TAATTAGAGACTTCCTATTCTCGAACTTTAAAGAGGAAGGCT | |
| | CTGTGGTGGTCGGAGGTTACTACCCCACAGAAGTGTGGTACA | |
| | ATTGCTCACGTACAGCCAGGACCACTGCCTTCCAGTACTTCA | |
| | ACAACATTCATGCCTTCTACTTTGTCATGGAAGCCATGGAGA | |
| | ACTCCACTGGGAATGCCAGAGGAAAGCCTCTCCTCTTCCATG | |
| | TCCATGGAGAGCCTGTCTCTGTGATTATCTCAGCATATAGGGA | |
| | TGATGTGCAGCAGCGGCCGCTGCTTAAGCATGGCCTAGTGTG | |
| | CATTACTAAGAACCGACATATCAATTATGAGCAGTTCACCTCC | |
| | AACCAGTGGAACTCCACATGCACTGGTGCTGATAGGAAGATC | |
| | CCGTTCAGCGTTATCCCCACCGATAATGGCACAAAGATTTATG | |
| | GCCTAGAATGGAACGATGATTTTGTTACTGCCTACATATCAGG | |
| | AAGAAGTTACCACTTAAACATTAACACCAATTGGTTCAATAAT | |
| | GTTACACTTCTGTACTCTCGCAGCAGTACGGCCACTTGGGAG | |
| | TATTCGGCTGCATATGCCTACCAAGGTGTAAGCAACTTCACCT | |
| | ACTACAAGCTGAACAATACGAACGGTCTGAAGACTTATGAGC | |
| | TGTGCGAAGACTACGAGCACTGTACGGGCTATGCGACAAATG | |
| | TCTTCGCCCCGACGAGCGGCGGGTACATACCGGATGGCTTCT | |
| | CCTTCAACAACTGGTTCCTCCTTACCAATAGCTCCACTTTCGT | |
| | ATCAGGAAGATTTGTTACGAACCAACCCCTTCTCATTAACTGT | |
| | CTGTGGCCAGTGCCCTCCTTCGGAGTAGCTGCTCAAGAGTTC | |
| | TGTTTCGAGGGTGCACAGTTCAGCCAGTGTAATGGAGTGTCG | |
| | CTGAACAACACTGTGGACGTGATCAGGTTTAATTTGAACTTC | |
| | ACAGCTGATGTTCAGTCCGGCATGGGCGCGACTGTGTTCAGC | |
| | CTAAACACCACGGGTGGCGTCATCTTGGAGATTAGTTGTTAC | |
| | TCTGACACTGTGTCAGAGAGCAGCAGTTACTCCTACGGAGA | |
| | AATTCCTTTCGGCATCACAGACGGTCCCCGGTACTGCTATGTG | |
| | CTGTACAACGGAACTGCTTTGAAGTACCTGGGGACATTGCCA | |
| | CCTTCTGTGAAGGAAATAGCCATCTCTAAGTGGGGTCACTTT | |
| | TACATTAACGGCTATAATTTCTTTTCCACTTTCCCAATTGGATG | |
| | CATTAGCTTCAACCTGACAACAGGTGTGTCTGGAGCCTTCTG | |
| | GACCATCGCCTATACCTCTTACACAGAGGCTCTAGTACAGGT | |
| | GGAGAACACAGCTATAAAGAACGTGACGTACTGTAACAGTC | |
| | ACATAAACAATATCAAGTGTTCTCAGTTGACTGCGAACTTAA | |
| | ACAATGGGTTTTATCCAGTGGCGAGCTCGGAGGTGGGGTTTG | |
| | TAAACAAATCTGTGGTGCTGTTGCCCTCCTTCTTCACGTACAC | |
| | TGCAGTGAACATCACCATTGATTTGGGGATGAAACTGTCCGG | |
| | CTACGGGCAGCCTATAGCATCTACACTGAGCAATATCACACTG | |
| | CCCATGCAGGATAACAATACAGATGTGTACTGTATCCGCTCAA | |
| | ACCAGTTCTCTGTATACGTGCACAGTACATGCAAGAGCTCGC | |
| | TATGGGACAACATTTTCAACCAGGATTGTACTGATGTGCTTGA | |
| | AGCAACTGCAGTGATCAAAACAGGCACATGCCCGTTCAGCT | |
| | TTGATAAGCTCAACAACTACCTAACGTTCAACAAGTTCTGCT | |
| | TGAGCCTGTCTCCAGTAGGCGCCAATTGCAAGTTTGACGTTG | |
| | CAGCGCGAACACGGACAAACGAACAGGTAGTGCGGTCGCTC | |
| | TATGTTATCTACGAGGAGGGGGACAACATAGTCGGGGTTCCA | |
| | TCCGACAACTCAGGTTTGCACGACCTGAGTGTGCTCCATTTG | |
| | GACTCATGCACGGATTATAACATCTACGGGCGCACAGGTGTG | |
| | GGGATAATACGAAGAACAAACTCTACGCTATTGAGCGGGCTC | |
| | TACTACACCTCATTGAGTGGGGACCTGCTAGGGTTCAAGAAC | |
| | GTATCTGACGGTGTGATCTATAGCGTCACACCATGTGACGTAT | |
| | CAGCCCAAGCTGCTGTGATTGACGGGGCGATTGTGGGGGCTA | |
| | TGACTTCAATTAACAGCGAGCTCCTAGGCCTGACCCACTGGA | |
| | CTACCACCCCAAACTTCTACTACTACAGCATTTATAACTATAC | |
| | CAGTGAGCGCACCAGGGACACTGCCATTGACAGCAATGACG | |
| | TCGACTGCGAGCCTGTTATTACCTACAGCAACATCGGTGTTT | |
| | GTAAGAATGGAGCTCTAGTCTTCATAAACGTAACGCACTCTG | |
| | ATGGCGATGTTCAACCAATTTCCACTGGGAACGTAACCATAC | |
| | CCACCAACTTTACTATTTCCGTCCAGGTGGAGTACATGCAAG | |
| | TATATACCACGCCAGTGTCCATCGACTGCGCTCGGTATGTGTG | |
| | CAACGGTAACCCACGCTGCAATAAGCTGCTAACGCAGTACGT | |
| | CAGCGCCTGCCAGACAATAGAGCAGGCATTGGCAATGGGTG | |
| | CAAGGCTTGAAAACATGGAGGTGGACTCCATGTTGTTCGTGT | |
| | CTGAAAACGCTCTTAAACTAGCATCCGTGGAGGCATTCAACA | |
| | GTACTGAGAACTTGGACTCTATCTATAAGGAGTGGCCCTCCAT | |
| | TGGGGGCAGCTGGCTTGGAGGTCTAAAAGACATCCTGCCCA | |
| | GCCACAACTCCAAGAGGAAGTACGGGTCCGCTATAGAGGAC | |
| | CTCCTCTTTGACAAGGTTGTTACTTCTGGTCTTGGCACAGTG | |
| | GACGAAGACTACAAGAGGTGCACAGGAGGCTATGATATAGCT | |
| | GACCTGGTGTGTGCTCAATACTACAACGGTATAATGGTTCTCC | |
| | CAGGTGTGGCCAACGCTGACAAGATGACAATGTACACAGCC | |
| | TCTTTAGCTGGAGGCATTACCCTGGGAGCCCTTGGGGGTGGC | |
| | GCAGTGGCAATTCCATTTGCCGTTGCGGTGCAGGCCCGACTA | |
| | AACTATGTCGCACTTCAAACAGATGTGCTCAACAAGAACCAA | |
| | CAAATACTGGCCAACGCTTTCAACCAGGCCATTGGTAACATT | |
| | ACGCAGGCATTTGGCAAGGTGAATGACGCCATCCACCAGAC | |
| | CAGCCAGGGACTTGCCACAGTGGCCAAGGCCTTGGCAAAGG | |
| | TGCAGGATGTCGTGAACACACAGGGTCAGGCCCTCTCTCATT | |
| | TGACAGTGCAGCTTCAGAATAACTTCCAAGCAATCAGTTCAA | |
| | GCATCAGCGACATCTACAACCGGCTGGACCCCCCATCTGCAG | |
| | ATGCGCAGGTGGACAGGCTAATCACTGGACGCTTGACGGCA | |
| | CTAAATGCCTTTGTCAGCCAAACTCTGACCCGGCAAGCAGAG | |
| | GTGCGGGCCAGTAGACAACTGGCCAAAGACAAGGTCAACGA | |
| | GTGCGTCAGGTCCCAGTCCCAGCGTTTTGGATTCTGTGGGAA | |
| | CGGGACGCACCTGTTCTCATTAGCCAATGCTGCACCCAATGG | |
| | CATGATCTTTTTCCATACTGTTCTACTTCCTACTGCCTATGAAA | |
| | CCGTGACCGCTTGGAGCGGCATCTGCGCATCTGATGGCGATA | |
| | GGACCTTCGGGCTGGTCGTTAAGGATGTCCAGCTAACGCTGT | |
| | TCCGGAACTTGGATGACAAGTTCTACCTGACCCCCAGGACCA | |
| | TGTACCAGCCGAGAGTGGCAACGAGTTCTGACTTCGTGCAA | |
| | ATTGAGGGCTGTGACGTCCTGTTTGTTAATGCAACAGTGATC | |
| | GATCTGCCCAGTATCATACCAGATTACATAGACATAAACCAGA | |
| | CAGTCCAGGACATACTGGAGAATTACAGGCCAAACTGGACC | |
| | GTACCAGAGTTCACGCTGGACATATTCAACGCTACGTACCTC | |
| | AATTTGACTGGGGAAATTGATGACTTGGAGTTCAGGTCGGAG | |
| | AAGCTCCACAACACCACTGTGGAGCTGGCCATCCTGATTGAC | |
| | AACATCAACAACACTCTGGTGAACCTGGAGTGGCTAAATCGC | |
| | ATTGAAACCTATGTCAAGTGGCCTTGGTACGTTTGGCTACTG | |
| | ATCGGACTCGTGGTAGTCTTCTGCATACCACTCCTGCTATTTT | |
| | GCTGCTTCAGCACAGGGTGCTGTGGCTGCATTGGATGCCTAG | |
| | GTTCCTGCTGTCACAGTATCTGCAGCAGAAGACAATTCGAGA | |
| | ACTACGAGCCCATAGAAAAGGTCCACGTACAT | |
| 110 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag- |
| | AAGGATGATGATGACAAGGACTACAAAGACGACGACGACAA | S_ec(S2) |
| | GGACTACAAGGATGACGATGACAAGACCACCAACAATGAAT | |
| | GCATCCAGGTGAACGTGACCCAGCTGGCAGGCAATGAAAAT | |
| | TTGATCAGAGACTTCCTGTTCAGCAACTTCAAGGAGGAGGG | |
| | CAGTGTAGTGGTGGGAGGCTACTACCCAACAGAGGTGTGGT | |
| | ACAACTGCAGCAGAACAGCCAGAACCACAGCCTTCCAGTAC | |
| | TTCAACAACATCCACGCCTTCTACTTTGTGATGGAGGCCATG | |
| | GAAAACAGCACAGGAAATGCCAGAGGAAAACCCCTGCTCTT | |
| | CCACGTGCACGGAGAGCCCGTGTCAGTCATCATCAGCGCCTA | |
| | CAGAGATGACGTCCAGCAGCGGCCCCTGCTGAAGCATGGAC | |
| | TGGTCTGCATCACCAAGAACAGACACATCAACTACGAGCAG | |
| | TTCACCAGCAACCAGTGGAACAGCACCTGCACAGGAGCAGA | |
| | CAGAAAAATCCCCTTCAGCGTCATCCCCACAGACAACGGCA | |
| | CCAAAATCTATGGCCTGGAGTGGAATGATGACTTTGTGACAG | |
| | CCTATATCAGCGGCAGGAGCTACCACCTCAACATCAACACCA | |
| | ACTGGTTCAACAACGTCACCCTGCTCTACTCCAGATCCAGCA | |
| | CAGCCACCTGGGAGTACAGCGCCGCCTATGCCTACCAGGGA | |
| | GTCTCCAACTTCACCTACTACAAACTGAACAACACCAACGGC | |
| | CTGAAAACCTACGAGCTGTGTGAGGACTACGAGCACTGCAC | |
| | AGGCTATGCCACAAATGTGTTTGCCCCAACCAGCGGAGGCTA | |
| | CATCCCAGACGGCTTCTCCTTCAACAACTGGTTCCTCCTCAC | |
| | CAACTCCTCCACATTTGTGAGCGGCAGATTTGTGACCAACCA | |
| | GCCCCTGCTGATCAACTGCCTGTGGCCCGTGCCCAGCTTTGG | |
| | AGTGGCAGCCCAGGAGTTCTGCTTCGAGGGAGCCCAGTTCA | |
| | GCCAGTGCAACGGAGTCAGCCTGAACAACACAGTGGACGTG | |
| | ATCAGATTCAACCTGAACTTCACAGCAGACGTGCAGAGTGG | |
| | AATGGGAGCCACCGTCTTCAGCCTGAACACCACAGGAGGAG | |
| | TGATCCTGGAGATCAGCTGCTACAGCGACACAGTGAGCGAG | |
| | AGCAGCAGCTACAGCTACGGAGAGATCCCATTTGGCATCACA | |
| | GATGGCCCCAGGTACTGCTACGTCCTGTACAATGGAACAGCC | |
| | CTGAAATACCTGGGCACCCTCCCACCCAGCGTGAAGGAGAT | |
| | CGCCATCAGCAAGTGGGGCCACTTCTACATCAATGGCTACAA | |
| | CTTCTTCAGCACCTTCCCCATCGGCTGCATCTCCTTCAACCTG | |
| | ACCACAGGAGTGAGCGGGGCCTTCTGGACAATCGCCTACAC | |
| | ATCCTACACAGAAGCCCTGGTGCAGGTGGAGAACACAGCCA | |
| | TCAAAAACGTCACCTACTGCAACAGCCACATCAACAACATC | |
| | AAGTGCAGCCAGCTGACAGCCAACCTGAACAACGGCTTCTA | |
| | CCCAGTGGCCAGCTCAGAGGTGGGCTTCGTGAACAAGAGCG | |
| | TGGTGCTCCTGCCCAGCTTCTTCACCTACACAGCAGTGAACA | |
| | TCACAATTGACCTGGGCATGAAGCTGAGCGGCTACGGCCAG | |
| | CCAATTGCCAGCACCCTCTCCAACATCACCCTCCCCATGCAG | |
| | GACAATAACACAGATGTGTACTGCATCAGATCCAACCAGTTC | |
| | TCTGTCTACGTGCACAGCACCTGCAAAAGCAGCCTGTGGGA | |
| | CAACATCTTCAACCAGGACTGCACAGATGTCCTGGAGGCCA | |
| | CAGCCGTGATCAAAACAGGCACCTGCCCCTTCAGCTTTGACA | |
| | AACTCAACAACTACCTTACATTCAACAAATTCTGCCTCTCCCT | |
| | CAGCCCAGTGGGAGCCAACTGCAAGTTTGATGTGGCCGCCA | |
| | GGACCAGGACAAATGAACAAGTGGTCAGAAGCCTCTACGTC | |
| | ATCTACGAGGAGGGAGACAACATCGTGGGGGTCCCCAGCGA | |
| | CAACAGCGGCCTGCACGACCTGAGTGTGCTCCACCTGGACA | |
| | GCTGCACAGACTACAACATCTACGGCAGGACTGGGGTGGGC | |
| | ATCATCAGAAGAACCAACAGCACACTGCTGAGTGGCCTGTA | |
| | CTACACCAGCCTGAGTGGAGACTTGCTGGGCTTCAAGAATGT | |
| | GTCAGATGGGGTGATCTACAGTGTGACCCCCTGTGACGTGTC | |
| | TGCCCAGGCTGCAGTCATCGACGGAGCCATCGTGGGAGCCAT | |
| | GACCAGCATTAACAGCGAGCTGCTGGGCCTGACCCACTGGA | |
| | CCACCACCCCCAACTTCTACTACTACTCCATCTACAACTACAC | |
| | ATCAGAAAGAACAAGAGACACAGCCATCGACAGCAATGACG | |
| | TGGACTGTGAGCCAGTCATCACCTACAGCAACATCGGAGTGT | |
| | GCAAGAACGGAGCCCTGGTGTTCATCAACGTGACCCACAGC | |
| | GACGGAGATGTCCAGCCCATCAGCACAGGAAATGTGACCAT | |
| | CCCAACCAACTTCACCATCAGCGTCCAGGTGGAATACATGCA | |
| | GGTGTACACCACCCCAGTGTCCATCGACTGTGCCAGATACGT | |
| | GTGCAATGGAAACCCCAGATGCAACAAGCTCCTCACCCAGT | |
| | ACGTGTCAGCCTGCCAGACAATCGAGCAGGCCCTGGCCATG | |
| | GGAGCCAGGCTCGAGAACATGGAAGTGGACAGCATGCTGTT | |
| | TGTCTCAGAGAATGCCCTGAAACTGGCCAGCGTGGAGGCCT | |
| | TCAACAGCACAGAGAACCTGGACAGCATCTACAAGGAGTGG | |
| | CCATCAATCGGAGGCAGCTGGCTGGGAGGACTTAAGGACAT | |
| | CCTGCCAAGCCACAACAGCAAAAGAAAGTACGGCAGCGCC | |
| | ATTGAGGACCTGCTGTTTGACAAGGTGGTCACCTCCGGCCTG | |
| | GGCACAGTGGATGAGGACTACAAGAGATGCACCGGCGGCTA | |
| | TGACATTGCCGACCTGGTGTGTGCCCAGTACTACAATGGCAT | |
| | CATGGTGCTGCCTGGAGTGGCCAACGCCGACAAAATGACCA | |
| | TGTACACCGCCTCCCTGGCTGGAGGCATCACACTGGGAGCCC | |
| | TGGGGGGAGGAGCAGTGGCCATCCCCTTTGCAGTGGCTGTG | |
| | CAGGCCAGACTCAACTACGTGGCCCTGCAGACAGACGTGCT | |
| | CAACAAGAACCAGCAGATCCTGGCCAACGCTTTCAACCAGG | |
| | CTATCGGAAACATCACCCAGGCCTTTGGAAAAGTGAATGATG | |
| | CCATCCACCAGACCAGCCAGGGCCTGGCCACAGTGGCCAAG | |
| | GCCCTGGCCAAGGTGCAGGACGTGGTCAACACCCAGGGCCA | |
| | GGCCCTCAGTCACCTCACAGTACAGCTCCAGAACAACTTCC | |
| | AGGCAATCTCCTCCTCCATCAGCGACATCTACAACAGGCTGG | |
| | ACCCCCCAAGCGCTGATGCCCAGGTGGACAGACTGATCACA | |
| | GGAAGACTCACAGCCCTCAACGCATTTGTGTCCCAGACACT | |
| | GACCAGGCAGGCAGAGGTCAGGGCCAGCAGGCAGCTGGCC | |
| | AAGGACAAGGTGAATGAGTGCGTGAGGAGCCAGAGCCAGA | |
| | GATTTGGCTTCTGCGGAAACGGCACCCACCTGTTCAGCCTGG | |
| | CCAACGCCGCCCCCAACGGCATGATTTTCTTCCACACAGTCC | |
| | TCCTCCCCACAGCCTACGAAACAGTGACAGCCTGGTCAGGC | |
| | ATCTGTGCCAGCGACGGAGACAGAACCTTTGGCCTGGTGGT | |
| | GAAGGATGTGCAGCTCACCCTCTTCAGAAACCTGGATGACA | |
| | AGTTCTACCTCACCCCAAGAACCATGTACCAGCCCAGAGTGG | |
| | CCACAAGCAGCGACTTTGTGCAGATTGAGGGCTGTGACGTG | |
| | CTGTTTGTGAATGCAACAGTGATTGACCTCCCAAGCATCATC | |
| | CCAGATTACATCGACATCAACCAGACAGTGCAGGACATCCTG | |
| | GAGAACTACAGGCCCAACTGGACAGTGCCAGAGTTCACCCT | |
| | GGACATCTTCAACGCCACCTACCTGAACCTGACAGGAGAAAT | |
| | TGACGACCTGGAGTTCAGATCAGAAAAACTTCACAACACCA | |
| | CCGTGGAGCTTGCCATCCTCATTGACAACATTAACAACACAC | |
| | TGGTCAACCTGGAATGGCTGAACAGAATTGAAACCTACGTG | |
| | AAGTGGCCC | |
| 111 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag- |
| | CAAAGATGATGATGACAAGGACTACAAAGACGACGACGACA | S_ec(S3) |
| | AAGACTACAAGGACGATGACGACAAGACCACCAACAACGA | |
| | GTGCATCCAGGTGAACGTGACCCAGCTGGCAGGCAACGAGA | |
| | ACCTCATCAGAGACTTCCTCTTCTCCAACTTCAAGGAGGAGG | |
| | GCTCAGTGGTGGTCGGCGGCTACTACCCAACAGAGGTGTGG | |
| | TACAACTGCTCAAGGACCGCCAGAACCACAGCCTTCCAGTA | |
| | CTTCAACAACATCCACGCCTTCTACTTCGTGATGGAGGCCAT | |
| | GGAGAACTCCACCGGGAACGCCAGGGGCAAGCCACTACTCT | |
| | TCCACGTGCACGGAGAGCCAGTGAGCGTGATCATCTCAGCCT | |
| | ACAGGGACGACGTGCAGCAGCGCCCCCTGCTGAAGCATGGA | |
| | CTGGTGTGCATCACCAAGAACAGGCACATCAACTACGAGCA | |
| | GTTCACCAGCAACCAGTGGAACAGCACCTGCACCGGCGCAG | |
| | ACAGGAAGATCCCCTTCTCAGTGATCCCAACAGACAACGGA | |
| | ACCAAAATCTACGGCCTGGAGTGGAACGACGACTTCGTGAC | |
| | CGCCTACATCAGCGGCAGGTCCTACCATCTCAACATCAACAC | |
| | CAACTGGTTCAACAACGTCACCCTCCTCTACAGCAGGTCATC | |
| | CACAGCCACCTGGGAGTACTCAGCTGCCTATGCATACCAGGG | |
| | AGTCTCCAACTTCACATACTACAAACTCAACAACACCAACGG | |
| | CCTCAAGACCTACGAGCTGTGTGAGGACTACGAGCACTGCA | |
| | CCGGCTACGCAACAAACGTCTTCGCCCCAACCTCCGGAGGC | |
| | TACATCCCAGACGGCTTCTCCTTCAACAACTGGTTCCTCCTC | |
| | ACAAACAGCTCCACCTTCGTGTCAGGAAGGTTCGTGACCAA | |
| | CCAGCCCCTGCTCATCAACTGCCTCTGGCCCGTCCCCTCCTT | |
| | CGGAGTGGCCGCCCAGGAGTTCTGCTTCGAGGGAGCCCAGT | |
| | TCTCCCAGTGCAACGGAGTCTCCCTCAACAACACCGTGGAC | |
| | GTCATCAGATTCAACCTCAACTTCACAGCAGACGTCCAGAGC | |
| | GGCATGGGAGCCACCGTGTTCAGCCTGAACACCACAGGAGG | |
| | AGTGATCCTGGAGATCTCCTGCTACTCAGACACAGTGTCAGA | |
| | GTCCTCCTCCTACAGCTACGGAGAGATCCCATTCGGCATCAC | |
| | AGACGGCCCCAGATACTGCTACGTGCTGTACAACGGCACAG | |
| | CCCTGAAGTACCTGGGCACCCTCCCCCCATCAGTGAAGGAG | |
| | ATCGCCATCAGCAAGTGGGGCCACTTCTACATCAACGGCTAC | |
| | AACTTCTTCTCCACCTTCCCCATCGGCTGCATCAGCTTCAACC | |
| | TGACCACCGGAGTGTCCGGAGCCTTCTGGACCATCGCCTACA | |
| | CATCATACACCGAGGCCCTGGTGCAGGTGGAGAACACAGCC | |
| | ATAAAGAACGTGACCTACTGCAACAGCCACATCAACAACATC | |
| | AAGTGCTCCCAGCTGACAGCCAACCTGAACAACGGCTTCTA | |
| | CCCAGTGGCCTCCAGCGAGGTGGGCTTCGTGAACAAGAGCG | |
| | TGGTCCTACTCCCCTCCTTCTTCACCTACACAGCAGTCAACAT | |
| | CACAATTGACCTGGGCATGAAGCTGTCCGGCTACGGCCAGCC | |
| | AATCGCCAGCACCCTGTCCAACATCACCCTGCCAATGCAGGA | |
| | CAACAACACCGACGTCTACTGCATCAGAAGCAACCAGTTCTC | |
| | CGTGTACGTCCACTCCACCTGCAAGTCCTCCCTCTGGGACAA | |
| | CATCTTCAACCAGGACTGCACAGACGTGCTGGAGGCCACAG | |
| | CTGTGATCAAGACAGGAACCTGCCCTTTCTCATTCGACAAGC | |
| | TCAACAACTACCTGACCTTCAACAAGTTCTGCCTGAGCCTGT | |
| | CCCCAGTGGGAGCCAACTGCAAGTTCGACGTGGCCGCCAGA | |
| | ACCAGGACCAACGAGCAGGTGGTCAGAAGCCTGTACGTCAT | |
| | CTACGAGGAGGGAGACAACATCGTGGGAGTGCCCAGCGACA | |
| | ACTCAGGCCTGCACGACCTGAGCGTGCTGCACCTGGACTCC | |
| | TGCACAGACTACAACATCTACGGCAGGACAGGAGTGGGCAT | |
| | CATCAGGAGGACCAACAGCACACTGCTGTCCGGCCTCTACTA | |
| | CACCTCCCTGTCCGGAGACTTGCTGGGATTCAAGAACGTGTC | |
| | AGACGGAGTCATCTACAGCGTCACCCCATGTGACGTGAGCGC | |
| | CCAGGCAGCAGTGATAGACGGAGCCATCGTGGGAGCCATGA | |
| | CCTCAATCAACTCAGAACTGCTGGGCCTCACCCACTGGACA | |
| | ACAACACCCAACTTCTACTACTACTCCATCTACAACTACACAT | |
| | CAGAAAGAACAAGGGACACAGCAATCGACTCCAACGACGT | |
| | GGACTGTGAGCCAGTCATCACCTACTCCAACATCGGCGTGTG | |
| | CAAGAACGGAGCCCTGGTGTTCATCAACGTCACCCACTCAG | |
| | ACGGCGACGTCCAGCCAATCTCCACAGGAAACGTCACCATC | |
| | CCCACCAACTTCACCATCAGCGTGCAGGTGGAGTACATGCAG | |
| | GTCTACACCACCCCAGTCTCCATCGACTGTGCCAGGTACGTG | |
| | TGCAACGGCAACCCAAGATGCAACAAACTGCTGACCCAGTA | |
| | CGTGAGCGCCTGCCAGACCATCGAGCAGGCCCTGGCCATGG | |
| | GCGCCAGGCTGGAGAACATGGAGGTGGACAGCATGCTCTTT | |
| | GTGAGCGAGAACGCCCTGAAGCTTGCCAGCGTGGAGGCCTT | |
| | CAACAGCACCGAAAACCTGGACTCCATCTACAAAGAGTGGC | |
| | CCTCCATAGGAGGCTCCTGGCTGGGAGGCCTGAAGGACATCC | |
| | TCCCATCCCACAACAGCAAAAGAAAGTACGGCAGCGCCATC | |
| | GAAGACCTGCTGTTCGACAAGGTGGTCACCTCAGGACTGGG | |
| | CACAGTGGACGAGGACTACAAGAGGTGCACCGGAGGCTACG | |
| | ACATCGCAGACCTGGTCTGTGCCCAGTACTACAACGGCATCA | |
| | TGGTGCTCCCAGGCGTGGCCAACGCCGACAAGATGACCATG | |
| | TACACAGCAAGCCTGGCTGGAGGAATCACACTGGGAGCCCT | |
| | GGGAGGAGGGGCCGTGGCCATTCCATTCGCCGTGGCCGTGC | |
| | AGGCCAGACTGAACTACGTGGCCCTGCAGACAGACGTGCTA | |
| | AACAAGAACCAGCAGATCCTGGCCAACGCCTTCAACCAGGC | |
| | CATCGGCAACATCACCCAGGCCTTCGGCAAGGTGAACGACG | |
| | CAATCCACCAGACATCACAGGGCCTGGCAACAGTGGCCAAG | |
| | GCCCTGGCCAAGGTCCAGGACGTGGTGAACACCCAGGGCCA | |
| | GGCCCTCTCACACCTGACAGTCCAGCTGCAGAACAACTTCC | |
| | AGGCAATCTCCTCCTCCATCTCAGACATCTACAACAGACTGG | |
| | ACCCCCCCTCAGCCGACGCCCAGGTGGACAGACTCATCACA | |
| | GGCAGGCTGACCGCCCTCAACGCCTTCGTGTCCCAGACCCTC | |
| | ACCAGGCAGGCCGAGGTGAGGGCCAGCAGGCAGCTCGCCA | |
| | AGGACAAGGTGAACGAGTGCGTCAGAAGCCAGAGCCAGAG | |
| | GTTCGGCTTCTGTGGCAACGGCACCCACCTGTTCTCCCTGGC | |
| | CAACGCAGCCCCCAACGGCATGATCTTCTTCCACACAGTCCT | |
| | CCTCCCAACAGCATATGAGACAGTCACCGCCTGGTCAGGAAT | |
| | CTGTGCCTCAGACGGGGACAGAACCTTCGGCCTGGTGGTCA | |
| | AGGACGTGCAGCTGACACTCTTCAGAAACCTGGACGACAAA | |
| | TTCTACCTGACCCCCAGGACCATGTACCAGCCAAGGGTGGCC | |
| | ACCTCCTCAGACTTCGTGCAGATCGAGGGCTGTGACGTGCTC | |
| | TTCGTGAACGCCACCGTCATCGACCTCCCATCCATCATCCCA | |
| | GACTACATCGACATCAACCAGACAGTGCAGGACATCCTGGA | |
| | GAACTACCGCCCCAACTGGACCGTGCCAGAGTTCACCCTAG | |
| | ACATATTCAACGCCACCTACCTGAACCTGACAGGAGAAATTG | |
| | ACGACCTGGAGTTCAGATCAGAAAAGCTACACAACACCACC | |
| | GTGGAGTTAGCCATCCTCATAGACAACATTAACAACACCCTC | |
| | GTCAACCTGGAGTGGCTCAACAGGATTGAAACCTACGTGAA | |
| | GTGGCCC | |
| 112 | ATGCGTTTTGTAATGTCACCTACTGTACTACTACTACTACTCG | MHCIsp- |
| | GAGCACTAGCAGCACCTCAGACTTGGGCCGGATCAGACTATA | Flag- |
| | AAGACGACGATGACAAAGATTATAAAGATGATGACGACAAG | S_ec(S6) |
| | GACTACAAGGATGATGATGACAAGACCACAAATAACGAGTG | |
| | CATTCAGGTCAACGTCACCCAGCTGGCCGGTAACGAGAACC | |
| | TAATTAGAGACTTCCTATTCTCGAACTTTAAAGAGGAAGGCT | |
| | CTGTGGTGGTCGGAGGTTACTACCCCACAGAAGTGTGGTACA | |
| | ATTGCTCACGTACAGCCAGGACCACTGCCTTCCAGTACTTCA | |
| | ACAACATTCATGCCTTCTACTTTGTCATGGAAGCCATGGAGA | |
| | ACTCCACTGGGAATGCCAGAGGAAAGCCTCTCCTCTTCCATG | |
| | TCCATGGAGAGCCTGTCTCTGTGATTATCTCAGCATATAGGGA | |
| | TGATGTGCAGCAGCGGCCGCTGCTTAAGCATGGCCTAGTGTG | |
| | CATTACTAAGAACCGACATATCAATTATGAGCAGTTCACCTCC | |
| | AACCAGTGGAACTCCACATGCACTGGTGCTGATAGGAAGATC | |
| | CCGTTCAGCGTTATCCCCACCGATAATGGCACAAAGATTTATG | |
| | GCCTAGAATGGAACGATGATTTTGTTACTGCCTACATATCAGG | |
| | AAGAAGTTACCACTTAAACATTAACACCAATTGGTTCAATAAT | |
| | GTTACACTTCTGTACTCTCGCAGCAGTACGGCCACTTGGGAG | |
| | TATTCGGCTGCATATGCCTACCAAGGTGTAAGCAACTTCACCT | |
| | ACTACAAGCTGAACAATACGAACGGTCTGAAGACTTATGAGC | |
| | TGTGCGAAGACTACGAGCACTGTACGGGCTATGCGACAAATG | |
| | TCTTCGCCCCGACGAGCGGCGGGTACATACCGGATGGCTTCT | |
| | CCTTCAACAACTGGTTCCTCCTTACCAATAGCTCCACTTTCGT | |
| | ATCAGGAAGATTTGTTACGAACCAACCCCTTCTCATTAACTGT | |
| | CTGTGGCCAGTGCCCTCCTTCGGAGTAGCTGCTCAAGAGTTC | |
| | TGTTTCGAGGGTGCACAGTTCAGCCAGTGTAATGGAGTGTCG | |
| | CTGAACAACACTGTGGACGTGATCAGGTTTAATTTGAACTTC | |
| | ACAGCTGATGTTCAGTCCGGCATGGGCGCGACTGTGTTCAGC | |
| | CTAAACACCACGGGTGGCGTCATCTTGGAGATTAGTTGTTAC | |
| | TCTGACACTGTGTCAGAGAGCAGCAGTTACTCCTACGGAGA | |
| | AATTCCTTTCGGCATCACAGACGGTCCCCGGTACTGCTATGTG | |
| | CTGTACAACGGAACTGCTTTGAAGTACCTGGGGACATTGCCA | |
| | CCTTCTGTGAAGGAAATAGCCATCTCTAAGTGGGGTCACTTT | |
| | TACATTAACGGCTATAATTTCTTTTCCACTTTCCCAATTGGATG | |
| | CATTAGCTTCAACCTGACAACAGGTGTGTCTGGAGCCTTCTG | |
| | GACCATCGCCTATACCTCTTACACAGAGGCTCTAGTACAGGT | |
| | GGAGAACACAGCTATAAAGAACGTGACGTACTGTAACAGTC | |
| | ACATAAACAATATCAAGTGTTCTCAGTTGACTGCGAACTTAA | |
| | ACAATGGGTTTTATCCAGTGGCGAGCTCGGAGGTGGGGTTTG | |
| | TAAACAAATCTGTGGTGCTGTTGCCCTCCTTCTTCACGTACAC | |
| | TGCAGTGAACATCACCATTGATTTGGGGATGAAACTGTCCGG | |
| | CTACGGGCAGCCTATAGCATCTACACTGAGCAATATCACACTG | |
| | CCCATGCAGGATAACAATACAGATGTGTACTGTATCCGCTCAA | |
| | ACCAGTTCTCTGTATACGTGCACAGTACATGCAAGAGCTCGC | |
| | TATGGGACAACATTTTCAACCAGGATTGTACTGATGTGCTTGA | |
| | AGCAACTGCAGTGATCAAAACAGGCACATGCCCGTTCAGCT | |
| | TTGATAAGCTCAACAACTACCTAACGTTCAACAAGTTCTGCT | |
| | TGAGCCTGTCTCCAGTAGGCGCCAATTGCAAGTTTGACGTTG | |
| | CAGCGCGAACACGGACAAACGAACAGGTAGTGCGGTCGCTC | |
| | TATGTTATCTACGAGGAGGGGGACAACATAGTCGGGGTTCCA | |
| | TCCGACAACTCAGGTTTGCACGACCTGAGTGTGCTCCATTTG | |
| | GACTCATGCACGGATTATAACATCTACGGGCGCACAGGTGTG | |
| | GGGATAATACGAAGAACAAACTCTACGCTATTGAGCGGGCTC | |
| | TACTACACCTCATTGAGTGGGGACCTGCTAGGGTTCAAGAAC | |
| | GTATCTGACGGTGTGATCTATAGCGTCACACCATGTGACGTAT | |
| | CAGCCCAAGCTGCTGTGATTGACGGGGCGATTGTGGGGGCTA | |
| | TGACTTCAATTAACAGCGAGCTCCTAGGCCTGACCCACTGGA | |
| | CTACCACCCCAAACTTCTACTACTACAGCATTTATAACTATAC | |
| | CAGTGAGCGCACCAGGGACACTGCCATTGACAGCAATGACG | |
| | TCGACTGCGAGCCTGTTATTACCTACAGCAACATCGGTGTTT | |
| | GTAAGAATGGAGCTCTAGTCTTCATAAACGTAACGCACTCTG | |
| | ATGGCGATGTTCAACCAATTTCCACTGGGAACGTAACCATAC | |
| | CCACCAACTTTACTATTTCCGTCCAGGTGGAGTACATGCAAG | |
| | TATATACCACGCCAGTGTCCATCGACTGCGCTCGGTATGTGTG | |
| | CAACGGTAACCCACGCTGCAATAAGCTGCTAACGCAGTACGT | |
| | CAGCGCCTGCCAGACAATAGAGCAGGCATTGGCAATGGGTG | |
| | CAAGGCTTGAAAACATGGAGGTGGACTCCATGTTGTTCGTGT | |
| | CTGAAAACGCTCTTAAACTAGCATCCGTGGAGGCATTCAACA | |
| | GTACTGAGAACTTGGACTCTATCTATAAGGAGTGGCCCTCCAT | |
| | TGGGGGCAGCTGGCTTGGAGGTCTAAAAGACATCCTGCCCA | |
| | GCCACAACTCCAAGAGGAAGTACGGGTCCGCTATAGAGGAC | |
| | CTCCTCTTTGACAAGGTTGTTACTTCTGGTCTTGGCACAGTG | |
| | GACGAAGACTACAAGAGGTGCACAGGAGGCTATGATATAGCT | |
| | GACCTGGTGTGTGCTCAATACTACAACGGTATAATGGTTCTCC | |
| | CAGGTGTGGCCAACGCTGACAAGATGACAATGTACACAGCC | |
| | TCTTTAGCTGGAGGCATTACCCTGGGAGCCCTTGGGGGTGGC | |
| | GCAGTGGCAATTCCATTTGCCGTTGCGGTGCAGGCCCGACTA | |
| | AACTATGTCGCACTTCAAACAGATGTGCTCAACAAGAACCAA | |
| | CAAATACTGGCCAACGCTTTCAACCAGGCCATTGGTAACATT | |
| | ACGCAGGCATTTGGCAAGGTGAATGACGCCATCCACCAGAC | |
| | CAGCCAGGGACTTGCCACAGTGGCCAAGGCCTTGGCAAAGG | |
| | TGCAGGATGTCGTGAACACACAGGGTCAGGCCCTCTCTCATT | |
| | TGACAGTGCAGCTTCAGAATAACTTCCAAGCAATCAGTTCAA | |
| | GCATCAGCGACATCTACAACCGGCTGGACCCCCCATCTGCAG | |
| | ATGCGCAGGTGGACAGGCTAATCACTGGACGCTTGACGGCA | |
| | CTAAATGCCTTTGTCAGCCAAACTCTGACCCGGCAAGCAGAG | |
| | GTGCGGGCCAGTAGACAACTGGCCAAAGACAAGGTCAACGA | |
| | GTGCGTCAGGTCCCAGTCCCAGCGTTTTGGATTCTGTGGGAA | |
| | CGGGACGCACCTGTTCTCATTAGCCAATGCTGCACCCAATGG | |
| | CATGATCTTTTTCCATACTGTTCTACTTCCTACTGCCTATGAAA | |
| | CCGTGACCGCTTGGAGCGGCATCTGCGCATCTGATGGCGATA | |
| | GGACCTTCGGGCTGGTCGTTAAGGATGTCCAGCTAACGCTGT | |
| | TCCGGAACTTGGATGACAAGTTCTACCTGACCCCCAGGACCA | |
| | TGTACCAGCCGAGAGTGGCAACGAGTTCTGACTTCGTGCAA | |
| | ATTGAGGGCTGTGACGTCCTGTTTGTTAATGCAACAGTGATC | |
| | GATCTGCCCAGTATCATACCAGATTACATAGACATAAACCAGA | |
| | CAGTCCAGGACATACTGGAGAATTACAGGCCAAACTGGACC | |
| | GTACCAGAGTTCACGCTGGACATATTCAACGCTACGTACCTC | |
| | AATTTGACTGGGGAAATTGATGACTTGGAGTTCAGGTCGGAG | |
| | AAGCTCCACAACACCACTGTGGAGCTGGCCATCCTGATTGAC | |
| | AACATCAACAACACTCTGGTGAACCTGGAGTGGCTAAATCGC | |
| | ATTGAAACCTATGTCAAGTGGCCT | |
| 113 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag-SII(S2) |
| | AAGGATGATGATGACAAGGACTACAAAGACGACGACGACAA | |
| | GGACTACAAGGATGACGATGACAAGTGTGAGCCAGTCATCA | |
| | CCTACAGCAACATCGGAGTGTGCAAGAACGGAGCCCTGGTG | |
| | TTCATCAACGTGACCCACAGCGACGGAGATGTCCAGCCCATC | |
| | AGCACAGGAAATGTGACCATCCCAACCAACTTCACCATCAGC | |
| | GTCCAGGTGGAATACATGCAGGTGTACACCACCCCAGTGTCC | |
| | ATCGACTGTGCCAGATACGTGTGCAATGGAAACCCCAGATGC | |
| | AACAAGCTCCTCACCCAGTACGTGTCAGCCTGCCAGACAATC | |
| | GAGCAGGCCCTGGCCATGGGAGCCAGGCTCGAGAACATGGA | |
| | AGTGGACAGCATGCTGTTTGTCTCAGAGAATGCCCTGAAACT | |
| | GGCCAGCGTGGAGGCCTTCAACAGCACAGAGAACCTGGACA | |
| | GCATCTACAAGGAGTGGCCATCAATCGGAGGCAGCTGGCTG | |
| | GGAGGACTTAAGGACATCCTGCCAAGCCACAACAGCAAAAG | |
| | AAAGTACGGCAGCGCCATTGAGGACCTGCTGTTTGACAAGG | |
| | TGGTCACCTCCGGCCTGGGCACAGTGGATGAGGACTACAAG | |
| | AGATGCACCGGCGGCTATGACATTGCCGACCTGGTGTGTGCC | |
| | CAGTACTACAATGGCATCATGGTGCTGCCTGGAGTGGCCAAC | |
| | GCCGACAAAATGACCATGTACACCGCCTCCCTGGCTGGAGG | |
| | CATCACACTGGGAGCCCTGGGGGGAGGAGCAGTGGCCATCC | |
| | CCTTTGCAGTGGCTGTGCAGGCCAGACTCAACTACGTGGCCC | |
| | TGCAGACAGACGTGCTCAACAAGAACCAGCAGATCCTGGCC | |
| | AACGCTTTCAACCAGGCTATCGGAAACATCACCCAGGCCTTT | |
| | GGAAAAGTGAATGATGCCATCCACCAGACCAGCCAGGGCCT | |
| | GGCCACAGTGGCCAAGGCCCTGGCCAAGGTGCAGGACGTGG | |
| | TCAACACCCAGGGCCAGGCCCTCAGTCACCTCACAGTACAG | |
| | CTCCAGAACAACTTCCAGGCAATCTCCTCCTCCATCAGCGAC | |
| | ATCTACAACAGGCTGGACCCCCCAAGCGCTGATGCCCAGGT | |
| | GGACAGACTGATCACAGGAAGACTCACAGCCCTCAACGCAT | |
| | TTGTGTCCCAGACACTGACCAGGCAGGCAGAGGTCAGGGCC | |
| | AGCAGGCAGCTGGCCAAGGACAAGGTGAATGAGTGCGTGA | |
| | GGAGCCAGAGCCAGAGATTTGGCTTCTGCGGAAACGGCACC | |
| | CACCTGTTCAGCCTGGCCAACGCCGCCCCCAACGGCATGATT | |
| | TTCTTCCACACAGTCCTCCTCCCCACAGCCTACGAAACAGTG | |
| | ACAGCCTGGTCAGGCATCTGTGCCAGCGACGGAGACAGAAC | |
| | CTTTGGCCTGGTGGTGAAGGATGTGCAGCTCACCCTCTTCAG | |
| | AAACCTGGATGACAAGTTCTACCTCACCCCAAGAACCATGTA | |
| | CCAGCCCAGAGTGGCCACAAGCAGCGACTTTGTGCAGATTG | |
| | AGGGCTGTGACGTGCTGTTTGTGAATGCAACAGTGATTGACC | |
| | TCCCAAGCATCATCCCAGATTACATCGACATCAACCAGACAG | |
| | TGCAGGACATCCTGGAGAACTACAGGCCCAACTGGACAGTG | |
| | CCAGAGTTCACCCTGGACATCTTCAACGCCACCTACCTGAAC | |
| | CTGACAGGAGAAATTGACGACCTGGAGTTCAGATCAGAAAA | |
| | ACTTCACAACACCACCGTGGAGCTTGCCATCCTCATTGACAA | |
| | CATTAACAACACACTGGTCAACCTGGAATGGCTGAACAGAAT | |
| | TGAAACCTACGTGAAGTGGCCCTGGTATGTGTGGCTGCTGAT | |
| | TGGACTGGTGGTGGTGTTCTGCATCCCACTGCTGCTGTTCTG | |
| | CTGCTTCAGCACCGGCTGCTGTGGATGCATCGGCTGCTTGGG | |
| | CAGCTGCTGCCACAGCATCTGCAGCAGGAGGCAGTTTGAGA | |
| | ACTACGAACCAATTGAAAAAGTGCACGTCCAC | |
| 114 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag-SII(S3) |
| | CAAAGATGATGATGACAAGGACTACAAAGACGACGACGACA | |
| | AAGACTACAAGGACGATGACGACAAGTGTGAGCCAGTCATC | |
| | ACCTACTCCAACATCGGCGTGTGCAAGAACGGAGCCCTGGT | |
| | GTTCATCAACGTCACCCACTCAGACGGCGACGTCCAGCCAAT | |
| | CTCCACAGGAAACGTCACCATCCCCACCAACTTCACCATCAG | |
| | CGTGCAGGTGGAGTACATGCAGGTCTACACCACCCCAGTCTC | |
| | CATCGACTGTGCCAGGTACGTGTGCAACGGCAACCCAAGAT | |
| | GCAACAAACTGCTGACCCAGTACGTGAGCGCCTGCCAGACC | |
| | ATCGAGCAGGCCCTGGCCATGGGCGCCAGGCTGGAGAACAT | |
| | GGAGGTGGACAGCATGCTCTTTGTGAGCGAGAACGCCCTGA | |
| | AGCTTGCCAGCGTGGAGGCCTTCAACAGCACCGAAAACCTG | |
| | GACTCCATCTACAAAGAGTGGCCCTCCATAGGAGGCTCCTGG | |
| | CTGGGAGGCCTGAAGGACATCCTCCCATCCCACAACAGCAA | |
| | AAGAAAGTACGGCAGCGCCATCGAAGACCTGCTGTTCGACA | |
| | AGGTGGTCACCTCAGGACTGGGCACAGTGGACGAGGACTAC | |
| | AAGAGGTGCACCGGAGGCTACGACATCGCAGACCTGGTCTG | |
| | TGCCCAGTACTACAACGGCATCATGGTGCTCCCAGGCGTGGC | |
| | CAACGCCGACAAGATGACCATGTACACAGCAAGCCTGGCTG | |
| | GAGGAATCACACTGGGAGCCCTGGGAGGAGGGGCCGTGGCC | |
| | ATTCCATTCGCCGTGGCCGTGCAGGCCAGACTGAACTACGTG | |
| | GCCCTGCAGACAGACGTGCTAAACAAGAACCAGCAGATCCT | |
| | GGCCAACGCCTTCAACCAGGCCATCGGCAACATCACCCAGG | |
| | CCTTCGGCAAGGTGAACGACGCAATCCACCAGACATCACAG | |
| | GGCCTGGCAACAGTGGCCAAGGCCCTGGCCAAGGTCCAGGA | |
| | CGTGGTGAACACCCAGGGCCAGGCCCTCTCACACCTGACAG | |
| | TCCAGCTGCAGAACAACTTCCAGGCAATCTCCTCCTCCATCT | |
| | CAGACATCTACAACAGACTGGACCCCCCCTCAGCCGACGCC | |
| | CAGGTGGACAGACTCATCACAGGCAGGCTGACCGCCCTCAA | |
| | CGCCTTCGTGTCCCAGACCCTCACCAGGCAGGCCGAGGTGA | |
| | GGGCCAGCAGGCAGCTCGCCAAGGACAAGGTGAACGAGTG | |
| | CGTCAGAAGCCAGAGCCAGAGGTTCGGCTTCTGTGGCAACG | |
| | GCACCCACCTGTTCTCCCTGGCCAACGCAGCCCCCAACGGC | |
| | ATGATCTTCTTCCACACAGTCCTCCTCCCAACAGCATATGAGA | |
| | CAGTCACCGCCTGGTCAGGAATCTGTGCCTCAGACGGGGAC | |
| | AGAACCTTCGGCCTGGTGGTCAAGGACGTGCAGCTGACACT | |
| | CTTCAGAAACCTGGACGACAAATTCTACCTGACCCCCAGGAC | |
| | CATGTACCAGCCAAGGGTGGCCACCTCCTCAGACTTCGTGCA | |
| | GATCGAGGGCTGTGACGTGCTCTTCGTGAACGCCACCGTCAT | |
| | CGACCTCCCATCCATCATCCCAGACTACATCGACATCAACCA | |
| | GACAGTGCAGGACATCCTGGAGAACTACCGCCCCAACTGGA | |
| | CCGTGCCAGAGTTCACCCTAGACATATTCAACGCCACCTACC | |
| | TGAACCTGACAGGAGAAATTGACGACCTGGAGTTCAGATCA | |
| | GAAAAGCTACACAACACCACCGTGGAGTTAGCCATCCTCATA | |
| | GACAACATTAACAACACCCTCGTCAACCTGGAGTGGCTCAA | |
| | CAGGATTGAAACCTACGTGAAGTGGCCCTGGTACGTCTGGCT | |
| | CCTCATCGGCCTGGTGGTGGTCTTCTGCATCCCACTGCTGCT | |
| | GTTCTGCTGCTTCTCCACCGGCTGCTGTGGATGCATCGGCTG | |
| | CCTGGGCTCATGCTGCCACTCAATCTGCTCAAGGAGGCAGTT | |
| | TGAAAACTACGAGCCAATAGAAAAAGTCCACGTCCAC | |
| 115 | ATGCGTTTTGTAATGTCACCTACTGTACTACTACTACTACTCG | MHCIsp- |
| | GAGCACTAGCAGCACCTCAGACTTGGGCCGGATCAGACTATA | Flag-SII(S6) |
| | AAGACGACGATGACAAAGATTATAAAGATGATGACGACAAG | |
| | GACTACAAGGATGATGATGACAAGTGCGAGCCTGTTATTACC | |
| | TACAGCAACATCGGTGTTTGTAAGAATGGAGCTCTAGTCTTC | |
| | ATAAACGTAACGCACTCTGATGGCGATGTTCAACCAATTTCC | |
| | ACTGGGAACGTAACCATACCCACCAACTTTACTATTTCCGTCC | |
| | AGGTGGAGTACATGCAAGTATATACCACGCCAGTGTCCATCG | |
| | ACTGCGCTCGGTATGTGTGCAACGGTAACCCACGCTGCAATA | |
| | AGCTGCTAACGCAGTACGTCAGCGCCTGCCAGACAATAGAG | |
| | CAGGCATTGGCAATGGGTGCAAGGCTTGAAAACATGGAGGT | |
| | GGACTCCATGTTGTTCGTGTCTGAAAACGCTCTTAAACTAGC | |
| | ATCCGTGGAGGCATTCAACAGTACTGAGAACTTGGACTCTAT | |
| | CTATAAGGAGTGGCCCTCCATTGGGGGCAGCTGGCTTGGAGG | |
| | TCTAAAAGACATCCTGCCCAGCCACAACTCCAAGAGGAAGT | |
| | ACGGGTCCGCTATAGAGGACCTCCTCTTTGACAAGGTTGTTA | |
| | CTTCTGGTCTTGGCACAGTGGACGAAGACTACAAGAGGTGC | |
| | ACAGGAGGCTATGATATAGCTGACCTGGTGTGTGCTCAATACT | |
| | ACAACGGTATAATGGTTCTCCCAGGTGTGGCCAACGCTGACA | |
| | AGATGACAATGTACACAGCCTCTTTAGCTGGAGGCATTACCC | |
| | TGGGAGCCCTTGGGGGTGGCGCAGTGGCAATTCCATTTGCCG | |
| | TTGCGGTGCAGGCCCGACTAAACTATGTCGCACTTCAAACAG | |
| | ATGTGCTCAACAAGAACCAACAAATACTGGCCAACGCTTTCA | |
| | ACCAGGCCATTGGTAACATTACGCAGGCATTTGGCAAGGTGA | |
| | ATGACGCCATCCACCAGACCAGCCAGGGACTTGCCACAGTG | |
| | GCCAAGGCCTTGGCAAAGGTGCAGGATGTCGTGAACACACA | |
| | GGGTCAGGCCCTCTCTCATTTGACAGTGCAGCTTCAGAATAA | |
| | CTTCCAAGCAATCAGTTCAAGCATCAGCGACATCTACAACCG | |
| | GCTGGACCCCCCATCTGCAGATGCGCAGGTGGACAGGCTAAT | |
| | CACTGGACGCTTGACGGCACTAAATGCCTTTGTCAGCCAAAC | |
| | TCTGACCCGGCAAGCAGAGGTGCGGGCCAGTAGACAACTGG | |
| | CCAAAGACAAGGTCAACGAGTGCGTCAGGTCCCAGTCCCAG | |
| | CGTTTTGGATTCTGTGGGAACGGGACGCACCTGTTCTCATTA | |
| | GCCAATGCTGCACCCAATGGCATGATCTTTTTCCATACTGTTC | |
| | TACTTCCTACTGCCTATGAAACCGTGACCGCTTGGAGCGGCA | |
| | TCTGCGCATCTGATGGCGATAGGACCTTCGGGCTGGTCGTTA | |
| | AGGATGTCCAGCTAACGCTGTTCCGGAACTTGGATGACAAGT | |
| | TCTACCTGACCCCCAGGACCATGTACCAGCCGAGAGTGGCA | |
| | ACGAGTTCTGACTTCGTGCAAATTGAGGGCTGTGACGTCCTG | |
| | TTTGTTAATGCAACAGTGATCGATCTGCCCAGTATCATACCAG | |
| | ATTACATAGACATAAACCAGACAGTCCAGGACATACTGGAGA | |
| | ATTACAGGCCAAACTGGACCGTACCAGAGTTCACGCTGGAC | |
| | ATATTCAACGCTACGTACCTCAATTTGACTGGGGAAATTGATG | |
| | ACTTGGAGTTCAGGTCGGAGAAGCTCCACAACACCACTGTG | |
| | GAGCTGGCCATCCTGATTGACAACATCAACAACACTCTGGTG | |
| | AACCTGGAGTGGCTAAATCGCATTGAAACCTATGTCAAGTGG | |
| | CCTTGGTACGTTTGGCTACTGATCGGACTCGTGGTAGTCTTCT | |
| | GCATACCACTCCTGCTATTTTGCTGCTTCAGCACAGGGTGCTG | |
| | TGGCTGCATTGGATGCCTAGGTTCCTGCTGTCACAGTATCTGC | |
| | AGCAGAAGACAATTCGAGAACTACGAGCCCATAGAAAAGGT | |
| | CCACGTACAT | |
| 116 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag-S(S2)- |
| | AAGGATGATGATGACAAGGACTACAAAGACGACGACGACAA | MITD |
| | GGACTACAAGGATGACGATGACAAGACCACCAACAATGAAT | |
| | GCATCCAGGTGAACGTGACCCAGCTGGCAGGCAATGAAAAT | |
| | TTGATCAGAGACTTCCTGTTCAGCAACTTCAAGGAGGAGGG | |
| | CAGTGTAGTGGTGGGAGGCTACTACCCAACAGAGGTGTGGT | |
| | ACAACTGCAGCAGAACAGCCAGAACCACAGCCTTCCAGTAC | |
| | TTCAACAACATCCACGCCTTCTACTTTGTGATGGAGGCCATG | |
| | GAAAACAGCACAGGAAATGCCAGAGGAAAACCCCTGCTCTT | |
| | CCACGTGCACGGAGAGCCCGTGTCAGTCATCATCAGCGCCTA | |
| | CAGAGATGACGTCCAGCAGCGGCCCCTGCTGAAGCATGGAC | |
| | TGGTCTGCATCACCAAGAACAGACACATCAACTACGAGCAG | |
| | TTCACCAGCAACCAGTGGAACAGCACCTGCACAGGAGCAGA | |
| | CAGAAAAATCCCCTTCAGCGTCATCCCCACAGACAACGGCA | |
| | CCAAAATCTATGGCCTGGAGTGGAATGATGACTTTGTGACAG | |
| | CCTATATCAGCGGCAGGAGCTACCACCTCAACATCAACACCA | |
| | ACTGGTTCAACAACGTCACCCTGCTCTACTCCAGATCCAGCA | |
| | CAGCCACCTGGGAGTACAGCGCCGCCTATGCCTACCAGGGA | |
| | GTCTCCAACTTCACCTACTACAAACTGAACAACACCAACGGC | |
| | CTGAAAACCTACGAGCTGTGTGAGGACTACGAGCACTGCAC | |
| | AGGCTATGCCACAAATGTGTTTGCCCCAACCAGCGGAGGCTA | |
| | CATCCCAGACGGCTTCTCCTTCAACAACTGGTTCCTCCTCAC | |
| | CAACTCCTCCACATTTGTGAGCGGCAGATTTGTGACCAACCA | |
| | GCCCCTGCTGATCAACTGCCTGTGGCCCGTGCCCAGCTTTGG | |
| | AGTGGCAGCCCAGGAGTTCTGCTTCGAGGGAGCCCAGTTCA | |
| | GCCAGTGCAACGGAGTCAGCCTGAACAACACAGTGGACGTG | |
| | ATCAGATTCAACCTGAACTTCACAGCAGACGTGCAGAGTGG | |
| | AATGGGAGCCACCGTCTTCAGCCTGAACACCACAGGAGGAG | |
| | TGATCCTGGAGATCAGCTGCTACAGCGACACAGTGAGCGAG | |
| | AGCAGCAGCTACAGCTACGGAGAGATCCCATTTGGCATCACA | |
| | GATGGCCCCAGGTACTGCTACGTCCTGTACAATGGAACAGCC | |
| | CTGAAATACCTGGGCACCCTCCCACCCAGCGTGAAGGAGAT | |
| | CGCCATCAGCAAGTGGGGCCACTTCTACATCAATGGCTACAA | |
| | CTTCTTCAGCACCTTCCCCATCGGCTGCATCTCCTTCAACCTG | |
| | ACCACAGGAGTGAGCGGGGCCTTCTGGACAATCGCCTACAC | |
| | ATCCTACACAGAAGCCCTGGTGCAGGTGGAGAACACAGCCA | |
| | TCAAAAACGTCACCTACTGCAACAGCCACATCAACAACATC | |
| | AAGTGCAGCCAGCTGACAGCCAACCTGAACAACGGCTTCTA | |
| | CCCAGTGGCCAGCTCAGAGGTGGGCTTCGTGAACAAGAGCG | |
| | TGGTGCTCCTGCCCAGCTTCTTCACCTACACAGCAGTGAACA | |
| | TCACAATTGACCTGGGCATGAAGCTGAGCGGCTACGGCCAG | |
| | CCAATTGCCAGCACCCTCTCCAACATCACCCTCCCCATGCAG | |
| | GACAATAACACAGATGTGTACTGCATCAGATCCAACCAGTTC | |
| | TCTGTCTACGTGCACAGCACCTGCAAAAGCAGCCTGTGGGA | |
| | CAACATCTTCAACCAGGACTGCACAGATGTCCTGGAGGCCA | |
| | CAGCCGTGATCAAAACAGGCACCTGCCCCTTCAGCTTTGACA | |
| | AACTCAACAACTACCTTACATTCAACAAATTCTGCCTCTCCCT | |
| | CAGCCCAGTGGGAGCCAACTGCAAGTTTGATGTGGCCGCCA | |
| | GGACCAGGACAAATGAACAAGTGGTCAGAAGCCTCTACGTC | |
| | ATCTACGAGGAGGGAGACAACATCGTGGGGGTCCCCAGCGA | |
| | CAACAGCGGCCTGCACGACCTGAGTGTGCTCCACCTGGACA | |
| | GCTGCACAGACTACAACATCTACGGCAGGACTGGGGTGGGC | |
| | ATCATCAGAAGAACCAACAGCACACTGCTGAGTGGCCTGTA | |
| | CTACACCAGCCTGAGTGGAGACTTGCTGGGCTTCAAGAATGT | |
| | GTCAGATGGGGTGATCTACAGTGTGACCCCCTGTGACGTGTC | |
| | TGCCCAGGCTGCAGTCATCGACGGAGCCATCGTGGGAGCCAT | |
| | GACCAGCATTAACAGCGAGCTGCTGGGCCTGACCCACTGGA | |
| | CCACCACCCCCAACTTCTACTACTACTCCATCTACAACTACAC | |
| | ATCAGAAAGAACAAGAGACACAGCCATCGACAGCAATGACG | |
| | TGGACTGTGAGCCAGTCATCACCTACAGCAACATCGGAGTGT | |
| | GCAAGAACGGAGCCCTGGTGTTCATCAACGTGACCCACAGC | |
| | GACGGAGATGTCCAGCCCATCAGCACAGGAAATGTGACCAT | |
| | CCCAACCAACTTCACCATCAGCGTCCAGGTGGAATACATGCA | |
| | GGTGTACACCACCCCAGTGTCCATCGACTGTGCCAGATACGT | |
| | GTGCAATGGAAACCCCAGATGCAACAAGCTCCTCACCCAGT | |
| | ACGTGTCAGCCTGCCAGACAATCGAGCAGGCCCTGGCCATG | |
| | GGAGCCAGGCTCGAGAACATGGAAGTGGACAGCATGCTGTT | |
| | TGTCTCAGAGAATGCCCTGAAACTGGCCAGCGTGGAGGCCT | |
| | TCAACAGCACAGAGAACCTGGACAGCATCTACAAGGAGTGG | |
| | CCATCAATCGGAGGCAGCTGGCTGGGAGGACTTAAGGACAT | |
| | CCTGCCAAGCCACAACAGCAAAAGAAAGTACGGCAGCGCC | |
| | ATTGAGGACCTGCTGTTTGACAAGGTGGTCACCTCCGGCCTG | |
| | GGCACAGTGGATGAGGACTACAAGAGATGCACCGGCGGCTA | |
| | TGACATTGCCGACCTGGTGTGTGCCCAGTACTACAATGGCAT | |
| | CATGGTGCTGCCTGGAGTGGCCAACGCCGACAAAATGACCA | |
| | TGTACACCGCCTCCCTGGCTGGAGGCATCACACTGGGAGCCC | |
| | TGGGGGGAGGAGCAGTGGCCATCCCCTTTGCAGTGGCTGTG | |
| | CAGGCCAGACTCAACTACGTGGCCCTGCAGACAGACGTGCT | |
| | CAACAAGAACCAGCAGATCCTGGCCAACGCTTTCAACCAGG | |
| | CTATCGGAAACATCACCCAGGCCTTTGGAAAAGTGAATGATG | |
| | CCATCCACCAGACCAGCCAGGGCCTGGCCACAGTGGCCAAG | |
| | GCCCTGGCCAAGGTGCAGGACGTGGTCAACACCCAGGGCCA | |
| | GGCCCTCAGTCACCTCACAGTACAGCTCCAGAACAACTTCC | |
| | AGGCAATCTCCTCCTCCATCAGCGACATCTACAACAGGCTGG | |
| | ACCCCCCAAGCGCTGATGCCCAGGTGGACAGACTGATCACA | |
| | GGAAGACTCACAGCCCTCAACGCATTTGTGTCCCAGACACT | |
| | GACCAGGCAGGCAGAGGTCAGGGCCAGCAGGCAGCTGGCC | |
| | AAGGACAAGGTGAATGAGTGCGTGAGGAGCCAGAGCCAGA | |
| | GATTTGGCTTCTGCGGAAACGGCACCCACCTGTTCAGCCTGG | |
| | CCAACGCCGCCCCCAACGGCATGATTTTCTTCCACACAGTCC | |
| | TCCTCCCCACAGCCTACGAAACAGTGACAGCCTGGTCAGGC | |
| | ATCTGTGCCAGCGACGGAGACAGAACCTTTGGCCTGGTGGT | |
| | GAAGGATGTGCAGCTCACCCTCTTCAGAAACCTGGATGACA | |
| | AGTTCTACCTCACCCCAAGAACCATGTACCAGCCCAGAGTGG | |
| | CCACAAGCAGCGACTTTGTGCAGATTGAGGGCTGTGACGTG | |
| | CTGTTTGTGAATGCAACAGTGATTGACCTCCCAAGCATCATC | |
| | CCAGATTACATCGACATCAACCAGACAGTGCAGGACATCCTG | |
| | GAGAACTACAGGCCCAACTGGACAGTGCCAGAGTTCACCCT | |
| | GGACATCTTCAACGCCACCTACCTGAACCTGACAGGAGAAAT | |
| | TGACGACCTGGAGTTCAGATCAGAAAAACTTCACAACACCA | |
| | CCGTGGAGCTTGCCATCCTCATTGACAACATTAACAACACAC | |
| | TGGTCAACCTGGAATGGCTGAACAGAATTGAAACCTACGTG | |
| | AAGTGGCCCTGGTATGTGTGGCTGCTGATTGGACTGGTGGTG | |
| | GTGTTCTGCATCCCACTGCTGCTGTTCTGCTGCTTCAGCACC | |
| | GGCTGCTGTGGATGCATCGGCTGCTTGGGCAGCTGCTGCCAC | |
| | AGCATCTGCAGCAGGAGGCAGTTTGAGAACTACGAACCAAT | |
| | TGAAAAAGTGCACGTCCACTTCCTGGGCATCATCGCCGGCGT | |
| | GGTGGTCCTGGTGGTCACAGTGGTGGTGGGAGCTGTGATCT | |
| | GGAGAAAGAAGTGCAGCGGCAGGAAGGGCCCAAGCTACAG | |
| | CCACGCTGCCAGAGATGACTCCACCCAGGGCAGCGACAGCA | |
| | GCCTGATGGCCCCCAAGGTG | |
| 117 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag-S(S3)- |
| | CAAAGATGATGATGACAAGGACTACAAAGACGACGACGACA | MITD |
| | AAGACTACAAGGACGATGACGACAAGACCACCAACAACGA | |
| | GTGCATCCAGGTGAACGTGACCCAGCTGGCAGGCAACGAGA | |
| | ACCTCATCAGAGACTTCCTCTTCTCCAACTTCAAGGAGGAGG | |
| | GCTCAGTGGTGGTCGGCGGCTACTACCCAACAGAGGTGTGG | |
| | TACAACTGCTCAAGGACCGCCAGAACCACAGCCTTCCAGTA | |
| | CTTCAACAACATCCACGCCTTCTACTTCGTGATGGAGGCCAT | |
| | GGAGAACTCCACCGGGAACGCCAGGGGCAAGCCACTACTCT | |
| | TCCACGTGCACGGAGAGCCAGTGAGCGTGATCATCTCAGCCT | |
| | ACAGGGACGACGTGCAGCAGCGCCCCCTGCTGAAGCATGGA | |
| | CTGGTGTGCATCACCAAGAACAGGCACATCAACTACGAGCA | |
| | GTTCACCAGCAACCAGTGGAACAGCACCTGCACCGGCGCAG | |
| | ACAGGAAGATCCCCTTCTCAGTGATCCCAACAGACAACGGA | |
| | ACCAAAATCTACGGCCTGGAGTGGAACGACGACTTCGTGAC | |
| | CGCCTACATCAGCGGCAGGTCCTACCATCTCAACATCAACAC | |
| | CAACTGGTTCAACAACGTCACCCTCCTCTACAGCAGGTCATC | |
| | CACAGCCACCTGGGAGTACTCAGCTGCCTATGCATACCAGGG | |
| | AGTCTCCAACTTCACATACTACAAACTCAACAACACCAACGG | |
| | CCTCAAGACCTACGAGCTGTGTGAGGACTACGAGCACTGCA | |
| | CCGGCTACGCAACAAACGTCTTCGCCCCAACCTCCGGAGGC | |
| | TACATCCCAGACGGCTTCTCCTTCAACAACTGGTTCCTCCTC | |
| | ACAAACAGCTCCACCTTCGTGTCAGGAAGGTTCGTGACCAA | |
| | CCAGCCCCTGCTCATCAACTGCCTCTGGCCCGTCCCCTCCTT | |
| | CGGAGTGGCCGCCCAGGAGTTCTGCTTCGAGGGAGCCCAGT | |
| | TCTCCCAGTGCAACGGAGTCTCCCTCAACAACACCGTGGAC | |
| | GTCATCAGATTCAACCTCAACTTCACAGCAGACGTCCAGAGC | |
| | GGCATGGGAGCCACCGTGTTCAGCCTGAACACCACAGGAGG | |
| | AGTGATCCTGGAGATCTCCTGCTACTCAGACACAGTGTCAGA | |
| | GTCCTCCTCCTACAGCTACGGAGAGATCCCATTCGGCATCAC | |
| | AGACGGCCCCAGATACTGCTACGTGCTGTACAACGGCACAG | |
| | CCCTGAAGTACCTGGGCACCCTCCCCCCATCAGTGAAGGAG | |
| | ATCGCCATCAGCAAGTGGGGCCACTTCTACATCAACGGCTAC | |
| | AACTTCTTCTCCACCTTCCCCATCGGCTGCATCAGCTTCAACC | |
| | TGACCACCGGAGTGTCCGGAGCCTTCTGGACCATCGCCTACA | |
| | CATCATACACCGAGGCCCTGGTGCAGGTGGAGAACACAGCC | |
| | ATAAAGAACGTGACCTACTGCAACAGCCACATCAACAACATC | |
| | AAGTGCTCCCAGCTGACAGCCAACCTGAACAACGGCTTCTA | |
| | CCCAGTGGCCTCCAGCGAGGTGGGCTTCGTGAACAAGAGCG | |
| | TGGTCCTACTCCCCTCCTTCTTCACCTACACAGCAGTCAACAT | |
| | CACAATTGACCTGGGCATGAAGCTGTCCGGCTACGGCCAGCC | |
| | AATCGCCAGCACCCTGTCCAACATCACCCTGCCAATGCAGGA | |
| | CAACAACACCGACGTCTACTGCATCAGAAGCAACCAGTTCTC | |
| | CGTGTACGTCCACTCCACCTGCAAGTCCTCCCTCTGGGACAA | |
| | CATCTTCAACCAGGACTGCACAGACGTGCTGGAGGCCACAG | |
| | CTGTGATCAAGACAGGAACCTGCCCTTTCTCATTCGACAAGC | |
| | TCAACAACTACCTGACCTTCAACAAGTTCTGCCTGAGCCTGT | |
| | CCCCAGTGGGAGCCAACTGCAAGTTCGACGTGGCCGCCAGA | |
| | ACCAGGACCAACGAGCAGGTGGTCAGAAGCCTGTACGTCAT | |
| | CTACGAGGAGGGAGACAACATCGTGGGAGTGCCCAGCGACA | |
| | ACTCAGGCCTGCACGACCTGAGCGTGCTGCACCTGGACTCC | |
| | TGCACAGACTACAACATCTACGGCAGGACAGGAGTGGGCAT | |
| | CATCAGGAGGACCAACAGCACACTGCTGTCCGGCCTCTACTA | |
| | CACCTCCCTGTCCGGAGACTTGCTGGGATTCAAGAACGTGTC | |
| | AGACGGAGTCATCTACAGCGTCACCCCATGTGACGTGAGCGC | |
| | CCAGGCAGCAGTGATAGACGGAGCCATCGTGGGAGCCATGA | |
| | CCTCAATCAACTCAGAACTGCTGGGCCTCACCCACTGGACA | |
| | ACAACACCCAACTTCTACTACTACTCCATCTACAACTACACAT | |
| | CAGAAAGAACAAGGGACACAGCAATCGACTCCAACGACGT | |
| | GGACTGTGAGCCAGTCATCACCTACTCCAACATCGGCGTGTG | |
| | CAAGAACGGAGCCCTGGTGTTCATCAACGTCACCCACTCAG | |
| | ACGGCGACGTCCAGCCAATCTCCACAGGAAACGTCACCATC | |
| | CCCACCAACTTCACCATCAGCGTGCAGGTGGAGTACATGCAG | |
| | GTCTACACCACCCCAGTCTCCATCGACTGTGCCAGGTACGTG | |
| | TGCAACGGCAACCCAAGATGCAACAAACTGCTGACCCAGTA | |
| | CGTGAGCGCCTGCCAGACCATCGAGCAGGCCCTGGCCATGG | |
| | GCGCCAGGCTGGAGAACATGGAGGTGGACAGCATGCTCTTT | |
| | GTGAGCGAGAACGCCCTGAAGCTTGCCAGCGTGGAGGCCTT | |
| | CAACAGCACCGAAAACCTGGACTCCATCTACAAAGAGTGGC | |
| | CCTCCATAGGAGGCTCCTGGCTGGGAGGCCTGAAGGACATCC | |
| | TCCCATCCCACAACAGCAAAAGAAAGTACGGCAGCGCCATC | |
| | GAAGACCTGCTGTTCGACAAGGTGGTCACCTCAGGACTGGG | |
| | CACAGTGGACGAGGACTACAAGAGGTGCACCGGAGGCTACG | |
| | ACATCGCAGACCTGGTCTGTGCCCAGTACTACAACGGCATCA | |
| | TGGTGCTCCCAGGCGTGGCCAACGCCGACAAGATGACCATG | |
| | TACACAGCAAGCCTGGCTGGAGGAATCACACTGGGAGCCCT | |
| | GGGAGGAGGGGCCGTGGCCATTCCATTCGCCGTGGCCGTGC | |
| | AGGCCAGACTGAACTACGTGGCCCTGCAGACAGACGTGCTA | |
| | AACAAGAACCAGCAGATCCTGGCCAACGCCTTCAACCAGGC | |
| | CATCGGCAACATCACCCAGGCCTTCGGCAAGGTGAACGACG | |
| | CAATCCACCAGACATCACAGGGCCTGGCAACAGTGGCCAAG | |
| | GCCCTGGCCAAGGTCCAGGACGTGGTGAACACCCAGGGCCA | |
| | GGCCCTCTCACACCTGACAGTCCAGCTGCAGAACAACTTCC | |
| | AGGCAATCTCCTCCTCCATCTCAGACATCTACAACAGACTGG | |
| | ACCCCCCCTCAGCCGACGCCCAGGTGGACAGACTCATCACA | |
| | GGCAGGCTGACCGCCCTCAACGCCTTCGTGTCCCAGACCCTC | |
| | ACCAGGCAGGCCGAGGTGAGGGCCAGCAGGCAGCTCGCCA | |
| | AGGACAAGGTGAACGAGTGCGTCAGAAGCCAGAGCCAGAG | |
| | GTTCGGCTTCTGTGGCAACGGCACCCACCTGTTCTCCCTGGC | |
| | CAACGCAGCCCCCAACGGCATGATCTTCTTCCACACAGTCCT | |
| | CCTCCCAACAGCATATGAGACAGTCACCGCCTGGTCAGGAAT | |
| | CTGTGCCTCAGACGGGGACAGAACCTTCGGCCTGGTGGTCA | |
| | AGGACGTGCAGCTGACACTCTTCAGAAACCTGGACGACAAA | |
| | TTCTACCTGACCCCCAGGACCATGTACCAGCCAAGGGTGGCC | |
| | ACCTCCTCAGACTTCGTGCAGATCGAGGGCTGTGACGTGCTC | |
| | TTCGTGAACGCCACCGTCATCGACCTCCCATCCATCATCCCA | |
| | GACTACATCGACATCAACCAGACAGTGCAGGACATCCTGGA | |
| | GAACTACCGCCCCAACTGGACCGTGCCAGAGTTCACCCTAG | |
| | ACATATTCAACGCCACCTACCTGAACCTGACAGGAGAAATTG | |
| | ACGACCTGGAGTTCAGATCAGAAAAGCTACACAACACCACC | |
| | GTGGAGTTAGCCATCCTCATAGACAACATTAACAACACCCTC | |
| | GTCAACCTGGAGTGGCTCAACAGGATTGAAACCTACGTGAA | |
| | GTGGCCCTGGTACGTCTGGCTCCTCATCGGCCTGGTGGTGGT | |
| | CTTCTGCATCCCACTGCTGCTGTTCTGCTGCTTCTCCACCGGC | |
| | TGCTGTGGATGCATCGGCTGCCTGGGCTCATGCTGCCACTCA | |
| | ATCTGCTCAAGGAGGCAGTTTGAAAACTACGAGCCAATAGA | |
| | AAAAGTCCACGTCCACTTCCTGGGCATAATCGCCGGCGTGGT | |
| | GGTGCTGGTGGTCACAGTGGTGGTCGGAGCAGTGATCTGGA | |
| | GGAAGAAGTGCTCAGGGAGGAAGGGCCCATCCTACTCCCAC | |
| | GCCGCCAGGGACGACAGCACCCAGGGCTCAGACTCATCCCT | |
| | GATGGCCCCCAAGGTG | |
| 118 | ATGCGTTTTGTAATGTCACCTACTGTACTACTACTACTACTCG | MHCIsp- |
| | GAGCACTAGCAGCACCTCAGACTTGGGCCGGATCAGACTATA | Flag-S(S6)- |
| | AAGACGACGATGACAAAGATTATAAAGATGATGACGACAAG | MITD |
| | GACTACAAGGATGATGATGACAAGACCACAAATAACGAGTG | |
| | CATTCAGGTCAACGTCACCCAGCTGGCCGGTAACGAGAACC | |
| | TAATTAGAGACTTCCTATTCTCGAACTTTAAAGAGGAAGGCT | |
| | CTGTGGTGGTCGGAGGTTACTACCCCACAGAAGTGTGGTACA | |
| | ATTGCTCACGTACAGCCAGGACCACTGCCTTCCAGTACTTCA | |
| | ACAACATTCATGCCTTCTACTTTGTCATGGAAGCCATGGAGA | |
| | ACTCCACTGGGAATGCCAGAGGAAAGCCTCTCCTCTTCCATG | |
| | TCCATGGAGAGCCTGTCTCTGTGATTATCTCAGCATATAGGGA | |
| | TGATGTGCAGCAGCGGCCGCTGCTTAAGCATGGCCTAGTGTG | |
| | CATTACTAAGAACCGACATATCAATTATGAGCAGTTCACCTCC | |
| | AACCAGTGGAACTCCACATGCACTGGTGCTGATAGGAAGATC | |
| | CCGTTCAGCGTTATCCCCACCGATAATGGCACAAAGATTTATG | |
| | GCCTAGAATGGAACGATGATTTTGTTACTGCCTACATATCAGG | |
| | AAGAAGTTACCACTTAAACATTAACACCAATTGGTTCAATAAT | |
| | GTTACACTTCTGTACTCTCGCAGCAGTACGGCCACTTGGGAG | |
| | TATTCGGCTGCATATGCCTACCAAGGTGTAAGCAACTTCACCT | |
| | ACTACAAGCTGAACAATACGAACGGTCTGAAGACTTATGAGC | |
| | TGTGCGAAGACTACGAGCACTGTACGGGCTATGCGACAAATG | |
| | TCTTCGCCCCGACGAGCGGCGGGTACATACCGGATGGCTTCT | |
| | CCTTCAACAACTGGTTCCTCCTTACCAATAGCTCCACTTTCGT | |
| | ATCAGGAAGATTTGTTACGAACCAACCCCTTCTCATTAACTGT | |
| | CTGTGGCCAGTGCCCTCCTTCGGAGTAGCTGCTCAAGAGTTC | |
| | TGTTTCGAGGGTGCACAGTTCAGCCAGTGTAATGGAGTGTCG | |
| | CTGAACAACACTGTGGACGTGATCAGGTTTAATTTGAACTTC | |
| | ACAGCTGATGTTCAGTCCGGCATGGGCGCGACTGTGTTCAGC | |
| | CTAAACACCACGGGTGGCGTCATCTTGGAGATTAGTTGTTAC | |
| | TCTGACACTGTGTCAGAGAGCAGCAGTTACTCCTACGGAGA | |
| | AATTCCTTTCGGCATCACAGACGGTCCCCGGTACTGCTATGTG | |
| | CTGTACAACGGAACTGCTTTGAAGTACCTGGGGACATTGCCA | |
| | CCTTCTGTGAAGGAAATAGCCATCTCTAAGTGGGGTCACTTT | |
| | TACATTAACGGCTATAATTTCTTTTCCACTTTCCCAATTGGATG | |
| | CATTAGCTTCAACCTGACAACAGGTGTGTCTGGAGCCTTCTG | |
| | GACCATCGCCTATACCTCTTACACAGAGGCTCTAGTACAGGT | |
| | GGAGAACACAGCTATAAAGAACGTGACGTACTGTAACAGTC | |
| | ACATAAACAATATCAAGTGTTCTCAGTTGACTGCGAACTTAA | |
| | ACAATGGGTTTTATCCAGTGGCGAGCTCGGAGGTGGGGTTTG | |
| | TAAACAAATCTGTGGTGCTGTTGCCCTCCTTCTTCACGTACAC | |
| | TGCAGTGAACATCACCATTGATTTGGGGATGAAACTGTCCGG | |
| | CTACGGGCAGCCTATAGCATCTACACTGAGCAATATCACACTG | |
| | CCCATGCAGGATAACAATACAGATGTGTACTGTATCCGCTCAA | |
| | ACCAGTTCTCTGTATACGTGCACAGTACATGCAAGAGCTCGC | |
| | TATGGGACAACATTTTCAACCAGGATTGTACTGATGTGCTTGA | |
| | AGCAACTGCAGTGATCAAAACAGGCACATGCCCGTTCAGCT | |
| | TTGATAAGCTCAACAACTACCTAACGTTCAACAAGTTCTGCT | |
| | TGAGCCTGTCTCCAGTAGGCGCCAATTGCAAGTTTGACGTTG | |
| | CAGCGCGAACACGGACAAACGAACAGGTAGTGCGGTCGCTC | |
| | TATGTTATCTACGAGGAGGGGGACAACATAGTCGGGGTTCCA | |
| | TCCGACAACTCAGGTTTGCACGACCTGAGTGTGCTCCATTTG | |
| | GACTCATGCACGGATTATAACATCTACGGGCGCACAGGTGTG | |
| | GGGATAATACGAAGAACAAACTCTACGCTATTGAGCGGGCTC | |
| | TACTACACCTCATTGAGTGGGGACCTGCTAGGGTTCAAGAAC | |
| | GTATCTGACGGTGTGATCTATAGCGTCACACCATGTGACGTAT | |
| | CAGCCCAAGCTGCTGTGATTGACGGGGCGATTGTGGGGGCTA | |
| | TGACTTCAATTAACAGCGAGCTCCTAGGCCTGACCCACTGGA | |
| | CTACCACCCCAAACTTCTACTACTACAGCATTTATAACTATAC | |
| | CAGTGAGCGCACCAGGGACACTGCCATTGACAGCAATGACG | |
| | TCGACTGCGAGCCTGTTATTACCTACAGCAACATCGGTGTTT | |
| | GTAAGAATGGAGCTCTAGTCTTCATAAACGTAACGCACTCTG | |
| | ATGGCGATGTTCAACCAATTTCCACTGGGAACGTAACCATAC | |
| | CCACCAACTTTACTATTTCCGTCCAGGTGGAGTACATGCAAG | |
| | TATATACCACGCCAGTGTCCATCGACTGCGCTCGGTATGTGTG | |
| | CAACGGTAACCCACGCTGCAATAAGCTGCTAACGCAGTACGT | |
| | CAGCGCCTGCCAGACAATAGAGCAGGCATTGGCAATGGGTG | |
| | CAAGGCTTGAAAACATGGAGGTGGACTCCATGTTGTTCGTGT | |
| | CTGAAAACGCTCTTAAACTAGCATCCGTGGAGGCATTCAACA | |
| | GTACTGAGAACTTGGACTCTATCTATAAGGAGTGGCCCTCCAT | |
| | TGGGGGCAGCTGGCTTGGAGGTCTAAAAGACATCCTGCCCA | |
| | GCCACAACTCCAAGAGGAAGTACGGGTCCGCTATAGAGGAC | |
| | CTCCTCTTTGACAAGGTTGTTACTTCTGGTCTTGGCACAGTG | |
| | GACGAAGACTACAAGAGGTGCACAGGAGGCTATGATATAGCT | |
| | GACCTGGTGTGTGCTCAATACTACAACGGTATAATGGTTCTCC | |
| | CAGGTGTGGCCAACGCTGACAAGATGACAATGTACACAGCC | |
| | TCTTTAGCTGGAGGCATTACCCTGGGAGCCCTTGGGGGTGGC | |
| | GCAGTGGCAATTCCATTTGCCGTTGCGGTGCAGGCCCGACTA | |
| | AACTATGTCGCACTTCAAACAGATGTGCTCAACAAGAACCAA | |
| | CAAATACTGGCCAACGCTTTCAACCAGGCCATTGGTAACATT | |
| | ACGCAGGCATTTGGCAAGGTGAATGACGCCATCCACCAGAC | |
| | CAGCCAGGGACTTGCCACAGTGGCCAAGGCCTTGGCAAAGG | |
| | TGCAGGATGTCGTGAACACACAGGGTCAGGCCCTCTCTCATT | |
| | TGACAGTGCAGCTTCAGAATAACTTCCAAGCAATCAGTTCAA | |
| | GCATCAGCGACATCTACAACCGGCTGGACCCCCCATCTGCAG | |
| | ATGCGCAGGTGGACAGGCTAATCACTGGACGCTTGACGGCA | |
| | CTAAATGCCTTTGTCAGCCAAACTCTGACCCGGCAAGCAGAG | |
| | GTGCGGGCCAGTAGACAACTGGCCAAAGACAAGGTCAACGA | |
| | GTGCGTCAGGTCCCAGTCCCAGCGTTTTGGATTCTGTGGGAA | |
| | CGGGACGCACCTGTTCTCATTAGCCAATGCTGCACCCAATGG | |
| | CATGATCTTTTTCCATACTGTTCTACTTCCTACTGCCTATGAAA | |
| | CCGTGACCGCTTGGAGCGGCATCTGCGCATCTGATGGCGATA | |
| | GGACCTTCGGGCTGGTCGTTAAGGATGTCCAGCTAACGCTGT | |
| | TCCGGAACTTGGATGACAAGTTCTACCTGACCCCCAGGACCA | |
| | TGTACCAGCCGAGAGTGGCAACGAGTTCTGACTTCGTGCAA | |
| | ATTGAGGGCTGTGACGTCCTGTTTGTTAATGCAACAGTGATC | |
| | GATCTGCCCAGTATCATACCAGATTACATAGACATAAACCAGA | |
| | CAGTCCAGGACATACTGGAGAATTACAGGCCAAACTGGACC | |
| | GTACCAGAGTTCACGCTGGACATATTCAACGCTACGTACCTC | |
| | AATTTGACTGGGGAAATTGATGACTTGGAGTTCAGGTCGGAG | |
| | AAGCTCCACAACACCACTGTGGAGCTGGCCATCCTGATTGAC | |
| | AACATCAACAACACTCTGGTGAACCTGGAGTGGCTAAATCGC | |
| | ATTGAAACCTATGTCAAGTGGCCTTGGTACGTTTGGCTACTG | |
| | ATCGGACTCGTGGTAGTCTTCTGCATACCACTCCTGCTATTTT | |
| | GCTGCTTCAGCACAGGGTGCTGTGGCTGCATTGGATGCCTAG | |
| | GTTCCTGCTGTCACAGTATCTGCAGCAGAAGACAATTCGAGA | |
| | ACTACGAGCCCATAGAAAAGGTCCACGTACATTTCCTGGGGA | |
| | TAATCGCAGGAGTGGTTGTTCTAGTGGTGACCGTGGTAGTTG | |
| | GGGCAGTGATCTGGAGAAAGAAATGCTCTGGCCGTAAGGGA | |
| | CCATCCTACTCCCATGCAGCACGTGATGATTCTACCCAGGGC | |
| | AGCGACAGTTCATTGATGGCCCCTAAAGTC | |
| 119 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag- |
| | AAGGATGATGATGACAAGGACTACAAAGACGACGACGACAA | S_ec(S2)- |
| | GGACTACAAGGATGACGATGACAAGACCACCAACAATGAAT | MITD |
| | GCATCCAGGTGAACGTGACCCAGCTGGCAGGCAATGAAAAT | |
| | TTGATCAGAGACTTCCTGTTCAGCAACTTCAAGGAGGAGGG | |
| | CAGTGTAGTGGTGGGAGGCTACTACCCAACAGAGGTGTGGT | |
| | ACAACTGCAGCAGAACAGCCAGAACCACAGCCTTCCAGTAC | |
| | TTCAACAACATCCACGCCTTCTACTTTGTGATGGAGGCCATG | |
| | GAAAACAGCACAGGAAATGCCAGAGGAAAACCCCTGCTCTT | |
| | CCACGTGCACGGAGAGCCCGTGTCAGTCATCATCAGCGCCTA | |
| | CAGAGATGACGTCCAGCAGCGGCCCCTGCTGAAGCATGGAC | |
| | TGGTCTGCATCACCAAGAACAGACACATCAACTACGAGCAG | |
| | TTCACCAGCAACCAGTGGAACAGCACCTGCACAGGAGCAGA | |
| | CAGAAAAATCCCCTTCAGCGTCATCCCCACAGACAACGGCA | |
| | CCAAAATCTATGGCCTGGAGTGGAATGATGACTTTGTGACAG | |
| | CCTATATCAGCGGCAGGAGCTACCACCTCAACATCAACACCA | |
| | ACTGGTTCAACAACGTCACCCTGCTCTACTCCAGATCCAGCA | |
| | CAGCCACCTGGGAGTACAGCGCCGCCTATGCCTACCAGGGA | |
| | GTCTCCAACTTCACCTACTACAAACTGAACAACACCAACGGC | |
| | CTGAAAACCTACGAGCTGTGTGAGGACTACGAGCACTGCAC | |
| | AGGCTATGCCACAAATGTGTTTGCCCCAACCAGCGGAGGCTA | |
| | CATCCCAGACGGCTTCTCCTTCAACAACTGGTTCCTCCTCAC | |
| | CAACTCCTCCACATTTGTGAGCGGCAGATTTGTGACCAACCA | |
| | GCCCCTGCTGATCAACTGCCTGTGGCCCGTGCCCAGCTTTGG | |
| | AGTGGCAGCCCAGGAGTTCTGCTTCGAGGGAGCCCAGTTCA | |
| | GCCAGTGCAACGGAGTCAGCCTGAACAACACAGTGGACGTG | |
| | ATCAGATTCAACCTGAACTTCACAGCAGACGTGCAGAGTGG | |
| | AATGGGAGCCACCGTCTTCAGCCTGAACACCACAGGAGGAG | |
| | TGATCCTGGAGATCAGCTGCTACAGCGACACAGTGAGCGAG | |
| | AGCAGCAGCTACAGCTACGGAGAGATCCCATTTGGCATCACA | |
| | GATGGCCCCAGGTACTGCTACGTCCTGTACAATGGAACAGCC | |
| | CTGAAATACCTGGGCACCCTCCCACCCAGCGTGAAGGAGAT | |
| | CGCCATCAGCAAGTGGGGCCACTTCTACATCAATGGCTACAA | |
| | CTTCTTCAGCACCTTCCCCATCGGCTGCATCTCCTTCAACCTG | |
| | ACCACAGGAGTGAGCGGGGCCTTCTGGACAATCGCCTACAC | |
| | ATCCTACACAGAAGCCCTGGTGCAGGTGGAGAACACAGCCA | |
| | TCAAAAACGTCACCTACTGCAACAGCCACATCAACAACATC | |
| | AAGTGCAGCCAGCTGACAGCCAACCTGAACAACGGCTTCTA | |
| | CCCAGTGGCCAGCTCAGAGGTGGGCTTCGTGAACAAGAGCG | |
| | TGGTGCTCCTGCCCAGCTTCTTCACCTACACAGCAGTGAACA | |
| | TCACAATTGACCTGGGCATGAAGCTGAGCGGCTACGGCCAG | |
| | CCAATTGCCAGCACCCTCTCCAACATCACCCTCCCCATGCAG | |
| | GACAATAACACAGATGTGTACTGCATCAGATCCAACCAGTTC | |
| | TCTGTCTACGTGCACAGCACCTGCAAAAGCAGCCTGTGGGA | |
| | CAACATCTTCAACCAGGACTGCACAGATGTCCTGGAGGCCA | |
| | CAGCCGTGATCAAAACAGGCACCTGCCCCTTCAGCTTTGACA | |
| | AACTCAACAACTACCTTACATTCAACAAATTCTGCCTCTCCCT | |
| | CAGCCCAGTGGGAGCCAACTGCAAGTTTGATGTGGCCGCCA | |
| | GGACCAGGACAAATGAACAAGTGGTCAGAAGCCTCTACGTC | |
| | ATCTACGAGGAGGGAGACAACATCGTGGGGGTCCCCAGCGA | |
| | CAACAGCGGCCTGCACGACCTGAGTGTGCTCCACCTGGACA | |
| | GCTGCACAGACTACAACATCTACGGCAGGACTGGGGTGGGC | |
| | ATCATCAGAAGAACCAACAGCACACTGCTGAGTGGCCTGTA | |
| | CTACACCAGCCTGAGTGGAGACTTGCTGGGCTTCAAGAATGT | |
| | GTCAGATGGGGTGATCTACAGTGTGACCCCCTGTGACGTGTC | |
| | TGCCCAGGCTGCAGTCATCGACGGAGCCATCGTGGGAGCCAT | |
| | GACCAGCATTAACAGCGAGCTGCTGGGCCTGACCCACTGGA | |
| | CCACCACCCCCAACTTCTACTACTACTCCATCTACAACTACAC | |
| | ATCAGAAAGAACAAGAGACACAGCCATCGACAGCAATGACG | |
| | TGGACTGTGAGCCAGTCATCACCTACAGCAACATCGGAGTGT | |
| | GCAAGAACGGAGCCCTGGTGTTCATCAACGTGACCCACAGC | |
| | GACGGAGATGTCCAGCCCATCAGCACAGGAAATGTGACCAT | |
| | CCCAACCAACTTCACCATCAGCGTCCAGGTGGAATACATGCA | |
| | GGTGTACACCACCCCAGTGTCCATCGACTGTGCCAGATACGT | |
| | GTGCAATGGAAACCCCAGATGCAACAAGCTCCTCACCCAGT | |
| | ACGTGTCAGCCTGCCAGACAATCGAGCAGGCCCTGGCCATG | |
| | GGAGCCAGGCTCGAGAACATGGAAGTGGACAGCATGCTGTT | |
| | TGTCTCAGAGAATGCCCTGAAACTGGCCAGCGTGGAGGCCT | |
| | TCAACAGCACAGAGAACCTGGACAGCATCTACAAGGAGTGG | |
| | CCATCAATCGGAGGCAGCTGGCTGGGAGGACTTAAGGACAT | |
| | CCTGCCAAGCCACAACAGCAAAAGAAAGTACGGCAGCGCC | |
| | ATTGAGGACCTGCTGTTTGACAAGGTGGTCACCTCCGGCCTG | |
| | GGCACAGTGGATGAGGACTACAAGAGATGCACCGGCGGCTA | |
| | TGACATTGCCGACCTGGTGTGTGCCCAGTACTACAATGGCAT | |
| | CATGGTGCTGCCTGGAGTGGCCAACGCCGACAAAATGACCA | |
| | TGTACACCGCCTCCCTGGCTGGAGGCATCACACTGGGAGCCC | |
| | TGGGGGGAGGAGCAGTGGCCATCCCCTTTGCAGTGGCTGTG | |
| | CAGGCCAGACTCAACTACGTGGCCCTGCAGACAGACGTGCT | |
| | CAACAAGAACCAGCAGATCCTGGCCAACGCTTTCAACCAGG | |
| | CTATCGGAAACATCACCCAGGCCTTTGGAAAAGTGAATGATG | |
| | CCATCCACCAGACCAGCCAGGGCCTGGCCACAGTGGCCAAG | |
| | GCCCTGGCCAAGGTGCAGGACGTGGTCAACACCCAGGGCCA | |
| | GGCCCTCAGTCACCTCACAGTACAGCTCCAGAACAACTTCC | |
| | AGGCAATCTCCTCCTCCATCAGCGACATCTACAACAGGCTGG | |
| | ACCCCCCAAGCGCTGATGCCCAGGTGGACAGACTGATCACA | |
| | GGAAGACTCACAGCCCTCAACGCATTTGTGTCCCAGACACT | |
| | GACCAGGCAGGCAGAGGTCAGGGCCAGCAGGCAGCTGGCC | |
| | AAGGACAAGGTGAATGAGTGCGTGAGGAGCCAGAGCCAGA | |
| | GATTTGGCTTCTGCGGAAACGGCACCCACCTGTTCAGCCTGG | |
| | CCAACGCCGCCCCCAACGGCATGATTTTCTTCCACACAGTCC | |
| | TCCTCCCCACAGCCTACGAAACAGTGACAGCCTGGTCAGGC | |
| | ATCTGTGCCAGCGACGGAGACAGAACCTTTGGCCTGGTGGT | |
| | GAAGGATGTGCAGCTCACCCTCTTCAGAAACCTGGATGACA | |
| | AGTTCTACCTCACCCCAAGAACCATGTACCAGCCCAGAGTGG | |
| | CCACAAGCAGCGACTTTGTGCAGATTGAGGGCTGTGACGTG | |
| | CTGTTTGTGAATGCAACAGTGATTGACCTCCCAAGCATCATC | |
| | CCAGATTACATCGACATCAACCAGACAGTGCAGGACATCCTG | |
| | GAGAACTACAGGCCCAACTGGACAGTGCCAGAGTTCACCCT | |
| | GGACATCTTCAACGCCACCTACCTGAACCTGACAGGAGAAAT | |
| | TGACGACCTGGAGTTCAGATCAGAAAAACTTCACAACACCA | |
| | CCGTGGAGCTTGCCATCCTCATTGACAACATTAACAACACAC | |
| | TGGTCAACCTGGAATGGCTGAACAGAATTGAAACCTACGTG | |
| | AAGTGGCCCTTCCTGGGCATCATCGCCGGCGTGGTGGTCCTG | |
| | GTGGTCACAGTGGTGGTGGGAGCTGTGATCTGGAGAAAGAA | |
| | GTGCAGCGGCAGGAAGGGCCCAAGCTACAGCCACGCTGCCA | |
| | GAGATGACTCCACCCAGGGCAGCGACAGCAGCCTGATGGCC | |
| | CCCAAGGTG | |
| 120 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag- |
| | CAAAGATGATGATGACAAGGACTACAAAGACGACGACGACA | S_ec(S3)- |
| | AAGACTACAAGGACGATGACGACAAGACCACCAACAACGA | MITD |
| | GTGCATCCAGGTGAACGTGACCCAGCTGGCAGGCAACGAGA | |
| | ACCTCATCAGAGACTTCCTCTTCTCCAACTTCAAGGAGGAGG | |
| | GCTCAGTGGTGGTCGGCGGCTACTACCCAACAGAGGTGTGG | |
| | TACAACTGCTCAAGGACCGCCAGAACCACAGCCTTCCAGTA | |
| | CTTCAACAACATCCACGCCTTCTACTTCGTGATGGAGGCCAT | |
| | GGAGAACTCCACCGGGAACGCCAGGGGCAAGCCACTACTCT | |
| | TCCACGTGCACGGAGAGCCAGTGAGCGTGATCATCTCAGCCT | |
| | ACAGGGACGACGTGCAGCAGCGCCCCCTGCTGAAGCATGGA | |
| | CTGGTGTGCATCACCAAGAACAGGCACATCAACTACGAGCA | |
| | GTTCACCAGCAACCAGTGGAACAGCACCTGCACCGGCGCAG | |
| | ACAGGAAGATCCCCTTCTCAGTGATCCCAACAGACAACGGA | |
| | ACCAAAATCTACGGCCTGGAGTGGAACGACGACTTCGTGAC | |
| | CGCCTACATCAGCGGCAGGTCCTACCATCTCAACATCAACAC | |
| | CAACTGGTTCAACAACGTCACCCTCCTCTACAGCAGGTCATC | |
| | CACAGCCACCTGGGAGTACTCAGCTGCCTATGCATACCAGGG | |
| | AGTCTCCAACTTCACATACTACAAACTCAACAACACCAACGG | |
| | CCTCAAGACCTACGAGCTGTGTGAGGACTACGAGCACTGCA | |
| | CCGGCTACGCAACAAACGTCTTCGCCCCAACCTCCGGAGGC | |
| | TACATCCCAGACGGCTTCTCCTTCAACAACTGGTTCCTCCTC | |
| | ACAAACAGCTCCACCTTCGTGTCAGGAAGGTTCGTGACCAA | |
| | CCAGCCCCTGCTCATCAACTGCCTCTGGCCCGTCCCCTCCTT | |
| | CGGAGTGGCCGCCCAGGAGTTCTGCTTCGAGGGAGCCCAGT | |
| | TCTCCCAGTGCAACGGAGTCTCCCTCAACAACACCGTGGAC | |
| | GTCATCAGATTCAACCTCAACTTCACAGCAGACGTCCAGAGC | |
| | GGCATGGGAGCCACCGTGTTCAGCCTGAACACCACAGGAGG | |
| | AGTGATCCTGGAGATCTCCTGCTACTCAGACACAGTGTCAGA | |
| | GTCCTCCTCCTACAGCTACGGAGAGATCCCATTCGGCATCAC | |
| | AGACGGCCCCAGATACTGCTACGTGCTGTACAACGGCACAG | |
| | CCCTGAAGTACCTGGGCACCCTCCCCCCATCAGTGAAGGAG | |
| | ATCGCCATCAGCAAGTGGGGCCACTTCTACATCAACGGCTAC | |
| | AACTTCTTCTCCACCTTCCCCATCGGCTGCATCAGCTTCAACC | |
| | TGACCACCGGAGTGTCCGGAGCCTTCTGGACCATCGCCTACA | |
| | CATCATACACCGAGGCCCTGGTGCAGGTGGAGAACACAGCC | |
| | ATAAAGAACGTGACCTACTGCAACAGCCACATCAACAACATC | |
| | AAGTGCTCCCAGCTGACAGCCAACCTGAACAACGGCTTCTA | |
| | CCCAGTGGCCTCCAGCGAGGTGGGCTTCGTGAACAAGAGCG | |
| | TGGTCCTACTCCCCTCCTTCTTCACCTACACAGCAGTCAACAT | |
| | CACAATTGACCTGGGCATGAAGCTGTCCGGCTACGGCCAGCC | |
| | AATCGCCAGCACCCTGTCCAACATCACCCTGCCAATGCAGGA | |
| | CAACAACACCGACGTCTACTGCATCAGAAGCAACCAGTTCTC | |
| | CGTGTACGTCCACTCCACCTGCAAGTCCTCCCTCTGGGACAA | |
| | CATCTTCAACCAGGACTGCACAGACGTGCTGGAGGCCACAG | |
| | CTGTGATCAAGACAGGAACCTGCCCTTTCTCATTCGACAAGC | |
| | TCAACAACTACCTGACCTTCAACAAGTTCTGCCTGAGCCTGT | |
| | CCCCAGTGGGAGCCAACTGCAAGTTCGACGTGGCCGCCAGA | |
| | ACCAGGACCAACGAGCAGGTGGTCAGAAGCCTGTACGTCAT | |
| | CTACGAGGAGGGAGACAACATCGTGGGAGTGCCCAGCGACA | |
| | ACTCAGGCCTGCACGACCTGAGCGTGCTGCACCTGGACTCC | |
| | TGCACAGACTACAACATCTACGGCAGGACAGGAGTGGGCAT | |
| | CATCAGGAGGACCAACAGCACACTGCTGTCCGGCCTCTACTA | |
| | CACCTCCCTGTCCGGAGACTTGCTGGGATTCAAGAACGTGTC | |
| | AGACGGAGTCATCTACAGCGTCACCCCATGTGACGTGAGCGC | |
| | CCAGGCAGCAGTGATAGACGGAGCCATCGTGGGAGCCATGA | |
| | CCTCAATCAACTCAGAACTGCTGGGCCTCACCCACTGGACA | |
| | ACAACACCCAACTTCTACTACTACTCCATCTACAACTACACAT | |
| | CAGAAAGAACAAGGGACACAGCAATCGACTCCAACGACGT | |
| | GGACTGTGAGCCAGTCATCACCTACTCCAACATCGGCGTGTG | |
| | CAAGAACGGAGCCCTGGTGTTCATCAACGTCACCCACTCAG | |
| | ACGGCGACGTCCAGCCAATCTCCACAGGAAACGTCACCATC | |
| | CCCACCAACTTCACCATCAGCGTGCAGGTGGAGTACATGCAG | |
| | GTCTACACCACCCCAGTCTCCATCGACTGTGCCAGGTACGTG | |
| | TGCAACGGCAACCCAAGATGCAACAAACTGCTGACCCAGTA | |
| | CGTGAGCGCCTGCCAGACCATCGAGCAGGCCCTGGCCATGG | |
| | GCGCCAGGCTGGAGAACATGGAGGTGGACAGCATGCTCTTT | |
| | GTGAGCGAGAACGCCCTGAAGCTTGCCAGCGTGGAGGCCTT | |
| | CAACAGCACCGAAAACCTGGACTCCATCTACAAAGAGTGGC | |
| | CCTCCATAGGAGGCTCCTGGCTGGGAGGCCTGAAGGACATCC | |
| | TCCCATCCCACAACAGCAAAAGAAAGTACGGCAGCGCCATC | |
| | GAAGACCTGCTGTTCGACAAGGTGGTCACCTCAGGACTGGG | |
| | CACAGTGGACGAGGACTACAAGAGGTGCACCGGAGGCTACG | |
| | ACATCGCAGACCTGGTCTGTGCCCAGTACTACAACGGCATCA | |
| | TGGTGCTCCCAGGCGTGGCCAACGCCGACAAGATGACCATG | |
| | TACACAGCAAGCCTGGCTGGAGGAATCACACTGGGAGCCCT | |
| | GGGAGGAGGGGCCGTGGCCATTCCATTCGCCGTGGCCGTGC | |
| | AGGCCAGACTGAACTACGTGGCCCTGCAGACAGACGTGCTA | |
| | AACAAGAACCAGCAGATCCTGGCCAACGCCTTCAACCAGGC | |
| | CATCGGCAACATCACCCAGGCCTTCGGCAAGGTGAACGACG | |
| | CAATCCACCAGACATCACAGGGCCTGGCAACAGTGGCCAAG | |
| | GCCCTGGCCAAGGTCCAGGACGTGGTGAACACCCAGGGCCA | |
| | GGCCCTCTCACACCTGACAGTCCAGCTGCAGAACAACTTCC | |
| | AGGCAATCTCCTCCTCCATCTCAGACATCTACAACAGACTGG | |
| | ACCCCCCCTCAGCCGACGCCCAGGTGGACAGACTCATCACA | |
| | GGCAGGCTGACCGCCCTCAACGCCTTCGTGTCCCAGACCCTC | |
| | ACCAGGCAGGCCGAGGTGAGGGCCAGCAGGCAGCTCGCCA | |
| | AGGACAAGGTGAACGAGTGCGTCAGAAGCCAGAGCCAGAG | |
| | GTTCGGCTTCTGTGGCAACGGCACCCACCTGTTCTCCCTGGC | |
| | CAACGCAGCCCCCAACGGCATGATCTTCTTCCACACAGTCCT | |
| | CCTCCCAACAGCATATGAGACAGTCACCGCCTGGTCAGGAAT | |
| | CTGTGCCTCAGACGGGGACAGAACCTTCGGCCTGGTGGTCA | |
| | AGGACGTGCAGCTGACACTCTTCAGAAACCTGGACGACAAA | |
| | TTCTACCTGACCCCCAGGACCATGTACCAGCCAAGGGTGGCC | |
| | ACCTCCTCAGACTTCGTGCAGATCGAGGGCTGTGACGTGCTC | |
| | TTCGTGAACGCCACCGTCATCGACCTCCCATCCATCATCCCA | |
| | GACTACATCGACATCAACCAGACAGTGCAGGACATCCTGGA | |
| | GAACTACCGCCCCAACTGGACCGTGCCAGAGTTCACCCTAG | |
| | ACATATTCAACGCCACCTACCTGAACCTGACAGGAGAAATTG | |
| | ACGACCTGGAGTTCAGATCAGAAAAGCTACACAACACCACC | |
| | GTGGAGTTAGCCATCCTCATAGACAACATTAACAACACCCTC | |
| | GTCAACCTGGAGTGGCTCAACAGGATTGAAACCTACGTGAA | |
| | GTGGCCCTTCCTGGGCATAATCGCCGGCGTGGTGGTGCTGGT | |
| | GGTCACAGTGGTGGTCGGAGCAGTGATCTGGAGGAAGAAGT | |
| | GCTCAGGGAGGAAGGGCCCATCCTACTCCCACGCCGCCAGG | |
| | GACGACAGCACCCAGGGCTCAGACTCATCCCTGATGGCCCC | |
| | CAAGGTG | |
| 121 | ATGCGTTTTGTAATGTCACCTACTGTACTACTACTACTACTCG | MHCIsp- |
| | GAGCACTAGCAGCACCTCAGACTTGGGCCGGATCAGACTATA | Flag- |
| | AAGACGACGATGACAAAGATTATAAAGATGATGACGACAAG | S_ec(S6)- |
| | GACTACAAGGATGATGATGACAAGACCACAAATAACGAGTG | MITD |
| | CATTCAGGTCAACGTCACCCAGCTGGCCGGTAACGAGAACC | |
| | TAATTAGAGACTTCCTATTCTCGAACTTTAAAGAGGAAGGCT | |
| | CTGTGGTGGTCGGAGGTTACTACCCCACAGAAGTGTGGTACA | |
| | ATTGCTCACGTACAGCCAGGACCACTGCCTTCCAGTACTTCA | |
| | ACAACATTCATGCCTTCTACTTTGTCATGGAAGCCATGGAGA | |
| | ACTCCACTGGGAATGCCAGAGGAAAGCCTCTCCTCTTCCATG | |
| | TCCATGGAGAGCCTGTCTCTGTGATTATCTCAGCATATAGGGA | |
| | TGATGTGCAGCAGCGGCCGCTGCTTAAGCATGGCCTAGTGTG | |
| | CATTACTAAGAACCGACATATCAATTATGAGCAGTTCACCTCC | |
| | AACCAGTGGAACTCCACATGCACTGGTGCTGATAGGAAGATC | |
| | CCGTTCAGCGTTATCCCCACCGATAATGGCACAAAGATTTATG | |
| | GCCTAGAATGGAACGATGATTTTGTTACTGCCTACATATCAGG | |
| | AAGAAGTTACCACTTAAACATTAACACCAATTGGTTCAATAAT | |
| | GTTACACTTCTGTACTCTCGCAGCAGTACGGCCACTTGGGAG | |
| | TATTCGGCTGCATATGCCTACCAAGGTGTAAGCAACTTCACCT | |
| | ACTACAAGCTGAACAATACGAACGGTCTGAAGACTTATGAGC | |
| | TGTGCGAAGACTACGAGCACTGTACGGGCTATGCGACAAATG | |
| | TCTTCGCCCCGACGAGCGGCGGGTACATACCGGATGGCTTCT | |
| | CCTTCAACAACTGGTTCCTCCTTACCAATAGCTCCACTTTCGT | |
| | ATCAGGAAGATTTGTTACGAACCAACCCCTTCTCATTAACTGT | |
| | CTGTGGCCAGTGCCCTCCTTCGGAGTAGCTGCTCAAGAGTTC | |
| | TGTTTCGAGGGTGCACAGTTCAGCCAGTGTAATGGAGTGTCG | |
| | CTGAACAACACTGTGGACGTGATCAGGTTTAATTTGAACTTC | |
| | ACAGCTGATGTTCAGTCCGGCATGGGCGCGACTGTGTTCAGC | |
| | CTAAACACCACGGGTGGCGTCATCTTGGAGATTAGTTGTTAC | |
| | TCTGACACTGTGTCAGAGAGCAGCAGTTACTCCTACGGAGA | |
| | AATTCCTTTCGGCATCACAGACGGTCCCCGGTACTGCTATGTG | |
| | CTGTACAACGGAACTGCTTTGAAGTACCTGGGGACATTGCCA | |
| | CCTTCTGTGAAGGAAATAGCCATCTCTAAGTGGGGTCACTTT | |
| | TACATTAACGGCTATAATTTCTTTTCCACTTTCCCAATTGGATG | |
| | CATTAGCTTCAACCTGACAACAGGTGTGTCTGGAGCCTTCTG | |
| | GACCATCGCCTATACCTCTTACACAGAGGCTCTAGTACAGGT | |
| | GGAGAACACAGCTATAAAGAACGTGACGTACTGTAACAGTC | |
| | ACATAAACAATATCAAGTGTTCTCAGTTGACTGCGAACTTAA | |
| | ACAATGGGTTTTATCCAGTGGCGAGCTCGGAGGTGGGGTTTG | |
| | TAAACAAATCTGTGGTGCTGTTGCCCTCCTTCTTCACGTACAC | |
| | TGCAGTGAACATCACCATTGATTTGGGGATGAAACTGTCCGG | |
| | CTACGGGCAGCCTATAGCATCTACACTGAGCAATATCACACTG | |
| | CCCATGCAGGATAACAATACAGATGTGTACTGTATCCGCTCAA | |
| | ACCAGTTCTCTGTATACGTGCACAGTACATGCAAGAGCTCGC | |
| | TATGGGACAACATTTTCAACCAGGATTGTACTGATGTGCTTGA | |
| | AGCAACTGCAGTGATCAAAACAGGCACATGCCCGTTCAGCT | |
| | TTGATAAGCTCAACAACTACCTAACGTTCAACAAGTTCTGCT | |
| | TGAGCCTGTCTCCAGTAGGCGCCAATTGCAAGTTTGACGTTG | |
| | CAGCGCGAACACGGACAAACGAACAGGTAGTGCGGTCGCTC | |
| | TATGTTATCTACGAGGAGGGGGACAACATAGTCGGGGTTCCA | |
| | TCCGACAACTCAGGTTTGCACGACCTGAGTGTGCTCCATTTG | |
| | GACTCATGCACGGATTATAACATCTACGGGCGCACAGGTGTG | |
| | GGGATAATACGAAGAACAAACTCTACGCTATTGAGCGGGCTC | |
| | TACTACACCTCATTGAGTGGGGACCTGCTAGGGTTCAAGAAC | |
| | GTATCTGACGGTGTGATCTATAGCGTCACACCATGTGACGTAT | |
| | CAGCCCAAGCTGCTGTGATTGACGGGGCGATTGTGGGGGCTA | |
| | TGACTTCAATTAACAGCGAGCTCCTAGGCCTGACCCACTGGA | |
| | CTACCACCCCAAACTTCTACTACTACAGCATTTATAACTATAC | |
| | CAGTGAGCGCACCAGGGACACTGCCATTGACAGCAATGACG | |
| | TCGACTGCGAGCCTGTTATTACCTACAGCAACATCGGTGTTT | |
| | GTAAGAATGGAGCTCTAGTCTTCATAAACGTAACGCACTCTG | |
| | ATGGCGATGTTCAACCAATTTCCACTGGGAACGTAACCATAC | |
| | CCACCAACTTTACTATTTCCGTCCAGGTGGAGTACATGCAAG | |
| | TATATACCACGCCAGTGTCCATCGACTGCGCTCGGTATGTGTG | |
| | CAACGGTAACCCACGCTGCAATAAGCTGCTAACGCAGTACGT | |
| | CAGCGCCTGCCAGACAATAGAGCAGGCATTGGCAATGGGTG | |
| | CAAGGCTTGAAAACATGGAGGTGGACTCCATGTTGTTCGTGT | |
| | CTGAAAACGCTCTTAAACTAGCATCCGTGGAGGCATTCAACA | |
| | GTACTGAGAACTTGGACTCTATCTATAAGGAGTGGCCCTCCAT | |
| | TGGGGGCAGCTGGCTTGGAGGTCTAAAAGACATCCTGCCCA | |
| | GCCACAACTCCAAGAGGAAGTACGGGTCCGCTATAGAGGAC | |
| | CTCCTCTTTGACAAGGTTGTTACTTCTGGTCTTGGCACAGTG | |
| | GACGAAGACTACAAGAGGTGCACAGGAGGCTATGATATAGCT | |
| | GACCTGGTGTGTGCTCAATACTACAACGGTATAATGGTTCTCC | |
| | CAGGTGTGGCCAACGCTGACAAGATGACAATGTACACAGCC | |
| | TCTTTAGCTGGAGGCATTACCCTGGGAGCCCTTGGGGGTGGC | |
| | GCAGTGGCAATTCCATTTGCCGTTGCGGTGCAGGCCCGACTA | |
| | AACTATGTCGCACTTCAAACAGATGTGCTCAACAAGAACCAA | |
| | CAAATACTGGCCAACGCTTTCAACCAGGCCATTGGTAACATT | |
| | ACGCAGGCATTTGGCAAGGTGAATGACGCCATCCACCAGAC | |
| | CAGCCAGGGACTTGCCACAGTGGCCAAGGCCTTGGCAAAGG | |
| | TGCAGGATGTCGTGAACACACAGGGTCAGGCCCTCTCTCATT | |
| | TGACAGTGCAGCTTCAGAATAACTTCCAAGCAATCAGTTCAA | |
| | GCATCAGCGACATCTACAACCGGCTGGACCCCCCATCTGCAG | |
| | ATGCGCAGGTGGACAGGCTAATCACTGGACGCTTGACGGCA | |
| | CTAAATGCCTTTGTCAGCCAAACTCTGACCCGGCAAGCAGAG | |
| | GTGCGGGCCAGTAGACAACTGGCCAAAGACAAGGTCAACGA | |
| | GTGCGTCAGGTCCCAGTCCCAGCGTTTTGGATTCTGTGGGAA | |
| | CGGGACGCACCTGTTCTCATTAGCCAATGCTGCACCCAATGG | |
| | CATGATCTTTTTCCATACTGTTCTACTTCCTACTGCCTATGAAA | |
| | CCGTGACCGCTTGGAGCGGCATCTGCGCATCTGATGGCGATA | |
| | GGACCTTCGGGCTGGTCGTTAAGGATGTCCAGCTAACGCTGT | |
| | TCCGGAACTTGGATGACAAGTTCTACCTGACCCCCAGGACCA | |
| | TGTACCAGCCGAGAGTGGCAACGAGTTCTGACTTCGTGCAA | |
| | ATTGAGGGCTGTGACGTCCTGTTTGTTAATGCAACAGTGATC | |
| | GATCTGCCCAGTATCATACCAGATTACATAGACATAAACCAGA | |
| | CAGTCCAGGACATACTGGAGAATTACAGGCCAAACTGGACC | |
| | GTACCAGAGTTCACGCTGGACATATTCAACGCTACGTACCTC | |
| | AATTTGACTGGGGAAATTGATGACTTGGAGTTCAGGTCGGAG | |
| | AAGCTCCACAACACCACTGTGGAGCTGGCCATCCTGATTGAC | |
| | AACATCAACAACACTCTGGTGAACCTGGAGTGGCTAAATCGC | |
| | ATTGAAACCTATGTCAAGTGGCCTTTCCTGGGGATAATCGCA | |
| | GGAGTGGTTGTTCTAGTGGTGACCGTGGTAGTTGGGGCAGTG | |
| | ATCTGGAGAAAGAAATGCTCTGGCCGTAAGGGACCATCCTAC | |
| | TCCCATGCAGCACGTGATGATTCTACCCAGGGCAGCGACAGT | |
| | TCATTGATGGCCCCTAAAGTC | |
| 122 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag- |
| | AAGGATGATGATGACAAGGACTACAAAGACGACGACGACAA | SII(S2)- |
| | GGACTACAAGGATGACGATGACAAGTGTGAGCCAGTCATCA | MITD |
| | CCTACAGCAACATCGGAGTGTGCAAGAACGGAGCCCTGGTG | |
| | TTCATCAACGTGACCCACAGCGACGGAGATGTCCAGCCCATC | |
| | AGCACAGGAAATGTGACCATCCCAACCAACTTCACCATCAGC | |
| | GTCCAGGTGGAATACATGCAGGTGTACACCACCCCAGTGTCC | |
| | ATCGACTGTGCCAGATACGTGTGCAATGGAAACCCCAGATGC | |
| | AACAAGCTCCTCACCCAGTACGTGTCAGCCTGCCAGACAATC | |
| | GAGCAGGCCCTGGCCATGGGAGCCAGGCTCGAGAACATGGA | |
| | AGTGGACAGCATGCTGTTTGTCTCAGAGAATGCCCTGAAACT | |
| | GGCCAGCGTGGAGGCCTTCAACAGCACAGAGAACCTGGACA | |
| | GCATCTACAAGGAGTGGCCATCAATCGGAGGCAGCTGGCTG | |
| | GGAGGACTTAAGGACATCCTGCCAAGCCACAACAGCAAAAG | |
| | AAAGTACGGCAGCGCCATTGAGGACCTGCTGTTTGACAAGG | |
| | TGGTCACCTCCGGCCTGGGCACAGTGGATGAGGACTACAAG | |
| | AGATGCACCGGCGGCTATGACATTGCCGACCTGGTGTGTGCC | |
| | CAGTACTACAATGGCATCATGGTGCTGCCTGGAGTGGCCAAC | |
| | GCCGACAAAATGACCATGTACACCGCCTCCCTGGCTGGAGG | |
| | CATCACACTGGGAGCCCTGGGGGGAGGAGCAGTGGCCATCC | |
| | CCTTTGCAGTGGCTGTGCAGGCCAGACTCAACTACGTGGCCC | |
| | TGCAGACAGACGTGCTCAACAAGAACCAGCAGATCCTGGCC | |
| | AACGCTTTCAACCAGGCTATCGGAAACATCACCCAGGCCTTT | |
| | GGAAAAGTGAATGATGCCATCCACCAGACCAGCCAGGGCCT | |
| | GGCCACAGTGGCCAAGGCCCTGGCCAAGGTGCAGGACGTGG | |
| | TCAACACCCAGGGCCAGGCCCTCAGTCACCTCACAGTACAG | |
| | CTCCAGAACAACTTCCAGGCAATCTCCTCCTCCATCAGCGAC | |
| | ATCTACAACAGGCTGGACCCCCCAAGCGCTGATGCCCAGGT | |
| | GGACAGACTGATCACAGGAAGACTCACAGCCCTCAACGCAT | |
| | TTGTGTCCCAGACACTGACCAGGCAGGCAGAGGTCAGGGCC | |
| | AGCAGGCAGCTGGCCAAGGACAAGGTGAATGAGTGCGTGA | |
| | GGAGCCAGAGCCAGAGATTTGGCTTCTGCGGAAACGGCACC | |
| | CACCTGTTCAGCCTGGCCAACGCCGCCCCCAACGGCATGATT | |
| | TTCTTCCACACAGTCCTCCTCCCCACAGCCTACGAAACAGTG | |
| | ACAGCCTGGTCAGGCATCTGTGCCAGCGACGGAGACAGAAC | |
| | CTTTGGCCTGGTGGTGAAGGATGTGCAGCTCACCCTCTTCAG | |
| | AAACCTGGATGACAAGTTCTACCTCACCCCAAGAACCATGTA | |
| | CCAGCCCAGAGTGGCCACAAGCAGCGACTTTGTGCAGATTG | |
| | AGGGCTGTGACGTGCTGTTTGTGAATGCAACAGTGATTGACC | |
| | TCCCAAGCATCATCCCAGATTACATCGACATCAACCAGACAG | |
| | TGCAGGACATCCTGGAGAACTACAGGCCCAACTGGACAGTG | |
| | CCAGAGTTCACCCTGGACATCTTCAACGCCACCTACCTGAAC | |
| | CTGACAGGAGAAATTGACGACCTGGAGTTCAGATCAGAAAA | |
| | ACTTCACAACACCACCGTGGAGCTTGCCATCCTCATTGACAA | |
| | CATTAACAACACACTGGTCAACCTGGAATGGCTGAACAGAAT | |
| | TGAAACCTACGTGAAGTGGCCCTGGTATGTGTGGCTGCTGAT | |
| | TGGACTGGTGGTGGTGTTCTGCATCCCACTGCTGCTGTTCTG | |
| | CTGCTTCAGCACCGGCTGCTGTGGATGCATCGGCTGCTTGGG | |
| | CAGCTGCTGCCACAGCATCTGCAGCAGGAGGCAGTTTGAGA | |
| | ACTACGAACCAATTGAAAAAGTGCACGTCCACTTCCTGGGC | |
| | ATCATCGCCGGCGTGGTGGTCCTGGTGGTCACAGTGGTGGTG | |
| | GGAGCTGTGATCTGGAGAAAGAAGTGCAGCGGCAGGAAGG | |
| | GCCCAAGCTACAGCCACGCTGCCAGAGATGACTCCACCCAG | |
| | GGCAGCGACAGCAGCCTGATGGCCCCCAAGGTG | |
| 123 | ATGAGATTTGTGATGAGCCCCACTGTGCTGCTGCTGCTGCTG | MHCIsp- |
| | GGAGCCCTGGCAGCCCCCCAGACCTGGGCTGGCTCAGACTA | Flag- |
| | CAAAGATGATGATGACAAGGACTACAAAGACGACGACGACA | SII(S3)- |
| | AAGACTACAAGGACGATGACGACAAGTGTGAGCCAGTCATC | MITD |
| | ACCTACTCCAACATCGGCGTGTGCAAGAACGGAGCCCTGGT | |
| | GTTCATCAACGTCACCCACTCAGACGGCGACGTCCAGCCAAT | |
| | CTCCACAGGAAACGTCACCATCCCCACCAACTTCACCATCAG | |
| | CGTGCAGGTGGAGTACATGCAGGTCTACACCACCCCAGTCTC | |
| | CATCGACTGTGCCAGGTACGTGTGCAACGGCAACCCAAGAT | |
| | GCAACAAACTGCTGACCCAGTACGTGAGCGCCTGCCAGACC | |
| | ATCGAGCAGGCCCTGGCCATGGGCGCCAGGCTGGAGAACAT | |
| | GGAGGTGGACAGCATGCTCTTTGTGAGCGAGAACGCCCTGA | |
| | AGCTTGCCAGCGTGGAGGCCTTCAACAGCACCGAAAACCTG | |
| | GACTCCATCTACAAAGAGTGGCCCTCCATAGGAGGCTCCTGG | |
| | CTGGGAGGCCTGAAGGACATCCTCCCATCCCACAACAGCAA | |
| | AAGAAAGTACGGCAGCGCCATCGAAGACCTGCTGTTCGACA | |
| | AGGTGGTCACCTCAGGACTGGGCACAGTGGACGAGGACTAC | |
| | AAGAGGTGCACCGGAGGCTACGACATCGCAGACCTGGTCTG | |
| | TGCCCAGTACTACAACGGCATCATGGTGCTCCCAGGCGTGGC | |
| | CAACGCCGACAAGATGACCATGTACACAGCAAGCCTGGCTG | |
| | GAGGAATCACACTGGGAGCCCTGGGAGGAGGGGCCGTGGCC | |
| | ATTCCATTCGCCGTGGCCGTGCAGGCCAGACTGAACTACGTG | |
| | GCCCTGCAGACAGACGTGCTAAACAAGAACCAGCAGATCCT | |
| | GGCCAACGCCTTCAACCAGGCCATCGGCAACATCACCCAGG | |
| | CCTTCGGCAAGGTGAACGACGCAATCCACCAGACATCACAG | |
| | GGCCTGGCAACAGTGGCCAAGGCCCTGGCCAAGGTCCAGGA | |
| | CGTGGTGAACACCCAGGGCCAGGCCCTCTCACACCTGACAG | |
| | TCCAGCTGCAGAACAACTTCCAGGCAATCTCCTCCTCCATCT | |
| | CAGACATCTACAACAGACTGGACCCCCCCTCAGCCGACGCC | |
| | CAGGTGGACAGACTCATCACAGGCAGGCTGACCGCCCTCAA | |
| | CGCCTTCGTGTCCCAGACCCTCACCAGGCAGGCCGAGGTGA | |
| | GGGCCAGCAGGCAGCTCGCCAAGGACAAGGTGAACGAGTG | |
| | CGTCAGAAGCCAGAGCCAGAGGTTCGGCTTCTGTGGCAACG | |
| | GCACCCACCTGTTCTCCCTGGCCAACGCAGCCCCCAACGGC | |
| | ATGATCTTCTTCCACACAGTCCTCCTCCCAACAGCATATGAGA | |
| | CAGTCACCGCCTGGTCAGGAATCTGTGCCTCAGACGGGGAC | |
| | AGAACCTTCGGCCTGGTGGTCAAGGACGTGCAGCTGACACT | |
| | CTTCAGAAACCTGGACGACAAATTCTACCTGACCCCCAGGAC | |
| | CATGTACCAGCCAAGGGTGGCCACCTCCTCAGACTTCGTGCA | |
| | GATCGAGGGCTGTGACGTGCTCTTCGTGAACGCCACCGTCAT | |
| | CGACCTCCCATCCATCATCCCAGACTACATCGACATCAACCA | |
| | GACAGTGCAGGACATCCTGGAGAACTACCGCCCCAACTGGA | |
| | CCGTGCCAGAGTTCACCCTAGACATATTCAACGCCACCTACC | |
| | TGAACCTGACAGGAGAAATTGACGACCTGGAGTTCAGATCA | |
| | GAAAAGCTACACAACACCACCGTGGAGTTAGCCATCCTCATA | |
| | GACAACATTAACAACACCCTCGTCAACCTGGAGTGGCTCAA | |
| | CAGGATTGAAACCTACGTGAAGTGGCCCTGGTACGTCTGGCT | |
| | CCTCATCGGCCTGGTGGTGGTCTTCTGCATCCCACTGCTGCT | |
| | GTTCTGCTGCTTCTCCACCGGCTGCTGTGGATGCATCGGCTG | |
| | CCTGGGCTCATGCTGCCACTCAATCTGCTCAAGGAGGCAGTT | |
| | TGAAAACTACGAGCCAATAGAAAAAGTCCACGTCCACTTCCT | |
| | GGGCATAATCGCCGGCGTGGTGGTGCTGGTGGTCACAGTGGT | |
| | GGTCGGAGCAGTGATCTGGAGGAAGAAGTGCTCAGGGAGG | |
| | AAGGGCCCATCCTACTCCCACGCCGCCAGGGACGACAGCAC | |
| | CCAGGGCTCAGACTCATCCCTGATGGCCCCCAAGGTG | |
| 124 | ATGCGTTTTGTAATGTCACCTACTGTACTACTACTACTACTCG | MHCIsp- |
| | GAGCACTAGCAGCACCTCAGACTTGGGCCGGATCAGACTATA | Flag- |
| | AAGACGACGATGACAAAGATTATAAAGATGATGACGACAAG | SII(S6)- |
| | GACTACAAGGATGATGATGACAAGTGCGAGCCTGTTATTACC | MITD |
| | TACAGCAACATCGGTGTTTGTAAGAATGGAGCTCTAGTCTTC | |
| | ATAAACGTAACGCACTCTGATGGCGATGTTCAACCAATTTCC | |
| | ACTGGGAACGTAACCATACCCACCAACTTTACTATTTCCGTCC | |
| | AGGTGGAGTACATGCAAGTATATACCACGCCAGTGTCCATCG | |
| | ACTGCGCTCGGTATGTGTGCAACGGTAACCCACGCTGCAATA | |
| | AGCTGCTAACGCAGTACGTCAGCGCCTGCCAGACAATAGAG | |
| | CAGGCATTGGCAATGGGTGCAAGGCTTGAAAACATGGAGGT | |
| | GGACTCCATGTTGTTCGTGTCTGAAAACGCTCTTAAACTAGC | |
| | ATCCGTGGAGGCATTCAACAGTACTGAGAACTTGGACTCTAT | |
| | CTATAAGGAGTGGCCCTCCATTGGGGGCAGCTGGCTTGGAGG | |
| | TCTAAAAGACATCCTGCCCAGCCACAACTCCAAGAGGAAGT | |
| | ACGGGTCCGCTATAGAGGACCTCCTCTTTGACAAGGTTGTTA | |
| | CTTCTGGTCTTGGCACAGTGGACGAAGACTACAAGAGGTGC | |
| | ACAGGAGGCTATGATATAGCTGACCTGGTGTGTGCTCAATACT | |
| | ACAACGGTATAATGGTTCTCCCAGGTGTGGCCAACGCTGACA | |
| | AGATGACAATGTACACAGCCTCTTTAGCTGGAGGCATTACCC | |
| | TGGGAGCCCTTGGGGGTGGCGCAGTGGCAATTCCATTTGCCG | |
| | TTGCGGTGCAGGCCCGACTAAACTATGTCGCACTTCAAACAG | |
| | ATGTGCTCAACAAGAACCAACAAATACTGGCCAACGCTTTCA | |
| | ACCAGGCCATTGGTAACATTACGCAGGCATTTGGCAAGGTGA | |
| | ATGACGCCATCCACCAGACCAGCCAGGGACTTGCCACAGTG | |
| | GCCAAGGCCTTGGCAAAGGTGCAGGATGTCGTGAACACACA | |
| | GGGTCAGGCCCTCTCTCATTTGACAGTGCAGCTTCAGAATAA | |
| | CTTCCAAGCAATCAGTTCAAGCATCAGCGACATCTACAACCG | |
| | GCTGGACCCCCCATCTGCAGATGCGCAGGTGGACAGGCTAAT | |
| | CACTGGACGCTTGACGGCACTAAATGCCTTTGTCAGCCAAAC | |
| | TCTGACCCGGCAAGCAGAGGTGCGGGCCAGTAGACAACTGG | |
| | CCAAAGACAAGGTCAACGAGTGCGTCAGGTCCCAGTCCCAG | |
| | CGTTTTGGATTCTGTGGGAACGGGACGCACCTGTTCTCATTA | |
| | GCCAATGCTGCACCCAATGGCATGATCTTTTTCCATACTGTTC | |
| | TACTTCCTACTGCCTATGAAACCGTGACCGCTTGGAGCGGCA | |
| | TCTGCGCATCTGATGGCGATAGGACCTTCGGGCTGGTCGTTA | |
| | AGGATGTCCAGCTAACGCTGTTCCGGAACTTGGATGACAAGT | |
| | TCTACCTGACCCCCAGGACCATGTACCAGCCGAGAGTGGCA | |
| | ACGAGTTCTGACTTCGTGCAAATTGAGGGCTGTGACGTCCTG | |
| | TTTGTTAATGCAACAGTGATCGATCTGCCCAGTATCATACCAG | |
| | ATTACATAGACATAAACCAGACAGTCCAGGACATACTGGAGA | |
| | ATTACAGGCCAAACTGGACCGTACCAGAGTTCACGCTGGAC | |
| | ATATTCAACGCTACGTACCTCAATTTGACTGGGGAAATTGATG | |
| | ACTTGGAGTTCAGGTCGGAGAAGCTCCACAACACCACTGTG | |
| | GAGCTGGCCATCCTGATTGACAACATCAACAACACTCTGGTG | |
| | AACCTGGAGTGGCTAAATCGCATTGAAACCTATGTCAAGTGG | |
| | CCTTGGTACGTTTGGCTACTGATCGGACTCGTGGTAGTCTTCT | |
| | GCATACCACTCCTGCTATTTTGCTGCTTCAGCACAGGGTGCTG | |
| | TGGCTGCATTGGATGCCTAGGTTCCTGCTGTCACAGTATCTGC | |
| | AGCAGAAGACAATTCGAGAACTACGAGCCCATAGAAAAGGT | |
| | CCACGTACATTTCCTGGGGATAATCGCAGGAGTGGTTGTTCT | |
| | AGTGGTGACCGTGGTAGTTGGGGCAGTGATCTGGAGAAAGA | |
| | AATGCTCTGGCCGTAAGGGACCATCCTACTCCCATGCAGCAC | |
| | GTGATGATTCTACCCAGGGCAGCGACAGTTCATTGATGGCCC | |
| | CTAAAGTC | |
| 125 | ATGCACCACCACCACCACCACCTGGTCTTCCTGCATGCTGTG | His-7a |
| | CTGGTGACTGTGCTCATCCTGCCCCTCATCGGCCGCATCCAG | |
| | CTGCTGGAGAGACTTCTCCTGAGCCACCTGCTGAACCTGACC | |
| | ACAGTCAGCAATGTCCTGGGGGTCCCAGACAGCAGCCTGCG | |
| | GGTCAACTGCCTGCAGCTGCTGAAGCCAGACTGCCTGGACT | |
| | TCAACATCCTGCACAAGGTGCTGGCAGAAACACGGCTGCTG | |
| | GTGGTGGTGCTGCGGGTCATCTTCCTGGTGCTGCTGGGCTTC | |
| | AGCTGCTACACCCTGCTGGGGGCCCTCTTC | |
| 126 | ATGGACTACAAGGATGATGATGACAAGGACTACAAAGACGA | Flag-3a |
| | CGACGACAAGGACTACAAGGATGACGATGACAAGGATGCTG | |
| | TGAAGAGCATCGGCATCTCTGTGGATGCTGTGCTGGATGAGC | |
| | TGGACAGCATTGCCTTTGCTGTCACCCTGAAGGTGCTCTTCA | |
| | ACAGCGGGAAGCTGCTGGTGTGCATCGGCTTCGGGGACACC | |
| | TTTGAGGAGGCTGAGCAGAAGGCCTATGCCAAGAGCAAGCT | |
| | GGTG | |
| 127 | ATGAGATTTGTGATGTCCCCCACTGTCCTGCTGCTGCTCCTGG | MHCIsp- |
| | GAGCCCTGGCAGCCCCCCAGACCTGGGCAGGCAGCTACCCA | HA- |
| | TACGATGTTCCAGACTACGCTTACCCATATGACGTGCCAGACT | M(HF1902)- |
| | ATGCCTACCCCTACGACGTGCCCGACTACGCAGAGAGATACT | MITD |
| | GTGCCATGAAGGACGACAGCAGCAACACCTGCATCAACGGC | |
| | ACCAACAGCAGCTGCCAGACCTGCTTTGAGAGAGGGGACCT | |
| | GATCTGGCACCTGGCCAACTGGAACTTCAGCTGGAGCGTGAT | |
| | CCTGATCGTGTTCATCACCGTGCTGCAGTATGGAAGACCCCA | |
| | GTTCAGCTGGCTGGTGTACGGCATCAAGATGCTGATCATGTG | |
| | GCTGCTGTGGCCCATCGTGCTGGCCCTGACCATCTTCAACGC | |
| | CTACAGCGAGTACCAGGTGTCCAGATACGTGATGTTTGGCTT | |
| | CAGCATCGCCGGGGCCGTGGTGACCTTCGCCCTGTGCATGAT | |
| | GTACTTCGTGAGGTCCATCCAGCTGTACAGGAGGACAAAGTC | |
| | ATGGTGGTCCTTCAACCCAGAAACCAATGCCATCCTGTGCGT | |
| | CAACGCACTGGGCAGAAGCTACGTCCTACCACTGGACGGCA | |
| | CTCCTACAGGAGTGACCCTGACCCTGCTGTCAGGCAATCTGT | |
| | ACGCAGAGGGGTTCAAGATGGCCGGTGGCCTGACCATCGAG | |
| | CATCTGCCTAAGTACGTGATGATCGCCACCCCTAGCAGGACA | |
| | ATCGTGTACACCCTGGTGGGAAAGCAGCTAAAGGCGACCAC | |
| | AGCCACAGGCTGGGCCTACTACGTGAAGTCCAAGGCAGGGG | |
| | ACTATTCAACCGAGGCCAGGACCGACAACCTGTCAGAGCAC | |
| | GAGAAGCTGCTGCACATGGTCTTCCTGGGCATCATCGCCGGC | |
| | GTGGTGGTCCTGGTGGTCACAGTGGTGGTGGGAGCTGTGAT | |
| | CTGGAGAAAGAAGTGCAGCGGCAGGAAGGGCCCAAGCTAC | |
| | AGCCACGCTGCCAGAGATGACTCCACCCAGGGCAGCGACAG | |
| | CAGCCTGATGGCCCCCAAGGTG | |
| 128 | ATGCACCACCACCACCACCACGCTACACAAGGACAGAGAGT | His- |
| | TAACTGGGGAGATGAACCAAGCAAGAGAAGAGACAGAAGC | N(HF1902) |
| | AACAGCAGAGGAAGAAAAAATGGAAACATCCCCCTGTCCTA | |
| | CTTCAACCCCATCACCCTGGAGAGCGGCAGCAAGTTCTGGA | |
| | ACATCTGCCCCAGGGACTTTGTGCCCAAGGGCATTGGAAAC | |
| | AAGGACCAGCAGATCGGCTACTGGAACAGACAGGTGCGCTA | |
| | CAGAATTGTGCGGGGCCAGAGGAAGGAGCTGCCCGAGAGAT | |
| | GGTTCTTCTACTTCTCTGGAACAGGCCCCCACGCTGATGCCA | |
| | AGTTCAAGGACAAGATTGATGGAGTGTTCTGGGTGGCCAGA | |
| | GATGGAGCCATGAACAAGCCCACCACCCTGGGCACCAGAGG | |
| | CACCAACAACGAGAGCAAGCCCCTGAAGTTTGATGGCAAGA | |
| | TCCCCCCCCAGTTCCAGCTGGAGGTGAACAGAAGCAGAAAC | |
| | AACAGCAGAAGCGGCAGCCAGCCCAGGAGCGTGTCCAGAA | |
| | GCAGAAGCCAGAGCAGAGGAAGACAGCAGAGCAACAACCA | |
| | GAACACCAACGTGGAGGACACCATCGTGGCCGTGCTGAGCA | |
| | AGCTGGGCGTGACAGACAAGCAGAGGAGCAGAAGCAAGTC | |
| | TGGAGAAAGAAACCAGAGCAAGCCCAGAGACACCACCCCC | |
| | AAGAATGCCAACAAGCACACCTGGAAGAAGACAGCTGGCA | |
| | AGGGGGACGTGACCAACTTCTATGGAGCCAGGAGCAGCAGC | |
| | GCCAACTTTGGAGACAGCGACCTGGTGGCCAATGGAAATGC | |
| | CGCCAAGTGCTACCCCCAGATCGCCGAGTGTGTGCCCAGCG | |
| | CCAGCAGCATCCTGTTTGGCAGCCAGTGGAGCGCCGAGGAG | |
| | GCCGGGGACCAGGTGAAGGTGACCCTGACCCACACCTACTA | |
| | CCTGCCCAAGGATGATGCCAAGACCAGCCAGTTCCTGGAGC | |
| | AGATTGATGCCTACAAGAGGCCCAGCGAGGTGGCCAAGGAC | |
| | CAGAGGCAGAGGAAGAGCAGAAGCAAGTCTGCTGACAAGA | |
| | AGCCAGAGGAGCTGTCGGTGACCCTCGTCGAGGCATACACC | |
| | GATGTCTTCGACGACACTCAGGTGGAGATGATCGACGAGGTC | |
| | ACCAAC | |
| 129 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag- |
| | AAGGATGATGATGACAAGGACTACAAAGACGACGACGACAA | SII(HF1902)- |
| | GGACTACAAGGATGACGATGACAAGTGCAGCAGCGTGATCA | MITD |
| | CCTACAGCAGCTTCGCCATCTGCAACACAGGAGAGATCAAGT | |
| | ACGTGAACGTGACCCACGTGGAGACTGTGGACGACAACATC | |
| | GGGGTGATCAAGCCCATCAGCACCGGCAACATCACCATCCCC | |
| | AAGAACTTCACAGTGGCCGTGCAGGCCGAGTACATCCAGAT | |
| | CCAGGTGAAGCCTGTGGTGGTGGACTGCGCCAAGTACGTGT | |
| | GCAATGGAAATGGCCACTGCCTGAACCTGCTGACCCAGTACA | |
| | CCTCTGCCTGCCAGACCATCGAGAACGCCCTGAACCTGGGC | |
| | GCCAGACTGGAGTCCCTGATGCTGTCTGAGATGGTGACCGTG | |
| | AGCGAGAGAAACCTGGACCTGGCCACCGTGGAGAAGTTCAA | |
| | CAGCACAGTGCTGGGCGGGGAGAAGCTGGGAGGCTTCTACT | |
| | TTGACGGCCTGAAGAGCCTGCTGCCTCCCACCATCGGCAAG | |
| | AGAAGCGCCGTGGAGGACCTGCTGTTCAACAAGGTGGTGAC | |
| | CAGCGGCCTGGGGACCGTGGATGATGACTACAAGAAGTGCA | |
| | GCGCCGGCACAGATGTGGCCGACCTGGCCTGTGCCCAGTACT | |
| | ACAACGGCATCATGGTGCTGCCTGGAGTGGTGGACCAGAAC | |
| | AAGATGGCCATGTACACCGCCAGCCTGATTGGAGGCATGGCC | |
| | CTGGGCAGCATCACCAGCGCCGTGGCCGTGCCCTTCGCCATG | |
| | CAGGTGCAGGCCAGACTGAACTACGTGGCCCTGCAGACAGA | |
| | TGTGCTGCAGGAGAACCAGAAGATCCTGGCCAACGCCTTCA | |
| | ACAACGCCATCGGCAACATCACCCTGGCCCTGGGGAAGGTG | |
| | AGCAACAGCATCACCACCATCTCTGGAGGCTTCCACACCATG | |
| | GCCAGCGCCCTGACCAAGATCCAGAGCGTGGTGAACCAGCA | |
| | GGGGGAGGCCCTGTCCCAGCTGACCAGCCAGCTGCAGAAGA | |
| | ACTTCCAGGCCATCTCCTCTTCCATTGCCGAGATCTACAACAG | |
| | ACTGGAGAAGGCTGAGGCCGACGCCCAGGTGGACAGGCTG | |
| | ATCACAGGAAGACTGGCCGCCCTGAACGCCTACGTGTCCCA | |
| | GACCCTGACCCAGTATGCCGAGGTGAAGGCCAGCAGACAGC | |
| | TGGCCATGGAGAAGGTGAATGAGTGTGTGAAGAGCCAGTCT | |
| | GACAGATACGGCTTCTGTGGAAATGGAACCCACCTGTTCAGC | |
| | CTGGTGAACTCTGCCCCTGACGGCCTGCTGTTCTTCCACACC | |
| | GTGCTGCTGCCCACAGAGTGGGAGGAGGTGACAGCCTGGAG | |
| | TGGCATCTGTGTGAATGACACCTACGCCTACGTGCTGAAAGA | |
| | CTTTGACTACAGCATCTTCAGCTACAACGGCACCTACATGGT | |
| | GACCCCCAGAAACATGTTCCAGCCCAGAAAGCCCCAGATGT | |
| | CAGACTTCGTGCAGATCACCAGATGCGAGGTGACCTTCCTGA | |
| | ACACAACCTACACCACCTTCCAGGAGATCGTGATCGACTACA | |
| | TCGACATCAACAAGACCATCGCCGACATGCTGGAGCAGTACA | |
| | ACCTGAACTACACAACCCCTGAGCTGAACCTGCAGCTGGAG | |
| | ATCTTCAACCAGACCAAGCTGAACCTGACCGCCGAGATCGA | |
| | CCAGCTGGAGCAGAGAGCCGACAACCTGACCAACATCGCCC | |
| | ACGAGCTGCAGCAGTACATCGACAACCTGAACAAGACCCTG | |
| | GTGGACCTGGAGTGGCTGAACAGAATTGAAACCTACGTGAA | |
| | GTGGCCCTGGTACGTGTGGCTGCTGATCGGCCTGGTGATCGT | |
| | GTTCTGCATCCCTCTGCTGCTGTTCTGCTGCCTGAGCACCGG | |
| | CTGCTGTGGCTGCTTCGGCTTCCTGGGCTCCTGCTGCCACTC | |
| | CCTGTGCAGCAGGAGGCAGTTTGAGTCCTACGAGCCCATCG | |
| | AGAAGGTGCACATCCACTTCCTGGGCATCATCGCCGGCGTGG | |
| | TGGTCCTGGTGGTCACAGTGGTGGTGGGAGCTGTGATCTGG | |
| | AGAAAGAAGTGCAGCGGCAGGAAGGGCCCAAGCTACAGCC | |
| | ACGCTGCCAGAGATGACTCCACCCAGGGCAGCGACAGCAGC | |
| | CTGATGGCCCCCAAGGTG | |
| 130 | ATGAGATTTGTGATGTCCCCCACTGTCCTGCTGCTGCTCCTGG | MHCIsp- |
| | GAGCCCTGGCAGCCCCCCAGACCTGGGCAGGCAGCTACCCA | HA- |
| | TACGATGTTCCAGACTACGCTTACCCATATGACGTGCCAGACT | M(SH2211)- |
| | ATGCCTACCCCTACGACGTGCCCGACTACGCAGAGAGATACT | MITD |
| | GTGCCATGCAGGACAGCGGCCTGCAGTGCATCAACGGCACC | |
| | AACAGCAGATGCCAGACCTGCTTTGAAAGAGGGGACCTGAT | |
| | CTGGCACCTGGCCAACTGGAACTTCTCCTGGAGCGTGATCCT | |
| | GATCGTGTTCATCACCGTGCTGCAGTACGGCAGACCACAGTT | |
| | CTCATGGCTTGTCTATGGCATCAAGATGCTGATTATGTGGCTG | |
| | CTTTGGCCTATCGTCCTGGCCCTGACCATCTTCAACGCCTACT | |
| | CTGAGTACCAGGTGTCAAGGTATGTCATGTTCGGCTTCTCAG | |
| | TGGCTGGAGCTGTGGTGACCTTTGCTCTGTGGATGATGTACT | |
| | TCGTGAGGTCCGTGCAGCTGTACAGGAGGACAAAGTCATGG | |
| | TGGTCCTTCAACCCAGAAACCAATGCCATCCTGTGCGTCAAC | |
| | GCACTGGGCAGAAGCTACGTCCTACCACTGGACGGCACTCC | |
| | TACAGGAGTGACCCTGACCCTGCTGTCAGGCAATCTGTACGC | |
| | AGAGGGGTTCAAGATGGCCGGTGGCCTGACCATCGAGCATC | |
| | TGCCTAAGTACGTGATGATCGCCACCCCTAGCAGGACAATCG | |
| | TGTACACCCTGGTGGGAAAGCAGCTAAAGGCGACCACAGCC | |
| | ACAGGCTGGGCCTACTACGTGAAGTCCAAGGCAGGGGACTA | |
| | TTCAACCGAGGCCAGGACAGACAACCTGAGCGAGCACGAG | |
| | AAGCTGCTGCACATGGTGTTCCTGGGCATCATCGCCGGCGTG | |
| | GTGGTCCTGGTGGTCACAGTGGTGGTGGGAGCTGTGATCTG | |
| | GAGAAAGAAGTGCAGCGGCAGGAAGGGCCCAAGCTACAGC | |
| | CACGCTGCCAGAGATGACTCCACCCAGGGCAGCGACAGCAG | |
| | CCTGATGGCCCCCAAGGTG | |
| 131 | ATGCACCACCACCACCACCACGCTACACAAGGACAGAGAGT | His- |
| | TAACTGGGGAGATGAACCAAGCAAGAGAAGAGGAAGAAGC | N(SH2211) |
| | AACAGCAGAGGAAGAAAGAACAATGACATCCCCCTGTCCTT | |
| | CTACAACCCCATCACCCTGGAGCAGGGCAGCAAGTTCTGGA | |
| | ACCTGTGCCCCAGGGACCTGGTGCCCAAGGGCATCGGCAAC | |
| | AAGGACCAGCAGATTGGCTACTGGAACAGACAGATCAGATA | |
| | CAGAATTGTGAAGGGCCAGAGGAAGGAGCTGGCCGAGAGG | |
| | TGGTTCTTCTACTTCCTGGGCACCGGCCCCCATGCTGATGCCA | |
| | AGTTCAAGGACAAGATTGATGGAGTGTTCTGGGTGGCCAGA | |
| | GATGGGGCCATGAACAAGCCCACCACCCTGGGCACCAGGGG | |
| | CACCAACAATGAGAGCAAGCCCCTGAGGTTTGATGGCAAGA | |
| | TCCCCCCCCAGTTCCAGCTGGAGGTGAACAGAAGCAGAAAC | |
| | AACAGCAGAAGCGGCAGCCAGAGCAGAAGTGTGTCCAGAA | |
| | ACAGAAGCCAGAGCAGAGGAAGACACCACAGCAACAACCA | |
| | GAACAACAATGTGGAGGACACCATCGTGGCCGTGCTGGAGA | |
| | AGCTGGGGGTGACAGACAAGCAGAGGAGCAGAAGCAAGCC | |
| | CAGAGAAAGAAGTGACAGCAAGCCCAGGGACACCACCCCC | |
| | AAGAATGCCAACAAGCACACCTGGAAGAAGACAGCTGGAA | |
| | AAGGAGATGTGACCACCTTCTATGGAGCCAGGAGCAGCAGC | |
| | GCCAACTTTGGAGACAGTGACCTGGTGGCCAATGGAAATGC | |
| | TGCCAAGTGCTACCCCCAGATTGCTGAGTGTGTGCCCTCTGT | |
| | GAGCAGCATCATCTTTGGCAGCCAGTGGTCAGCAGAGGAGG | |
| | CTGGGGACCAGGTGAAGGTGACCCTGACCCACACCTACTAC | |
| | CTGCCCAAGGATGATGCCAAGACCAGCCAGTTCCTGGAGCA | |
| | GATTGATGCCTACAAGAGGCCCAGCGAGGTGGCCAAGGACC | |
| | AGAGGCAGAGGAGGAGCCTGAGCAAGTCTGCTGACAAGAA | |
| | GCCAGAGGAGCTGTCGGTGACCCTCGTCGAGGCGTACACCG | |
| | ACGTCTTCGACGACACTCAGGTGGAGATGATCGACGAGGTC | |
| | ACCAAC | |
| 132 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag- |
| | AAGGATGATGATGACAAGGACTACAAAGACGACGACGACAA | SII(SH2211)- |
| | GGACTACAAGGATGACGATGACAAGTGTGAGCCAGTCATCA | MITD |
| | CCTACAGCAACATCGGAGTGTGCAAGAACGGAGCCCTGGTG | |
| | TTCATCAACGTGACCCACAGCGACGGAGATGTCCAGCCCATC | |
| | AGCACAGGAAATGTGACCATCCCAACCAACTTCACCATCAGC | |
| | GTCCAGGTGGAATACATGCAGGTGTACACCACCCCAGTGTCC | |
| | ATCGACTGTGCCAGATACGTGTGCAATGGAAACCCCAGATGC | |
| | AACAAGCTCCTCACCCAGTACGTGTCAGCCTGCCAGACAATC | |
| | GAGCAGGCCCTGGCCATGGGAGCCAGGCTCGAGAACATGGA | |
| | AGTGGACAGCATGCTGTTTGTCTCAGAGAATGCCCTGAAACT | |
| | GGCCAGCGTGGAGGCCTTCAACAGCACAGAGAACCTGGACC | |
| | CCATCTACAAGGAGTGGCCATCAATCGGAGGCAGCTGGCTGG | |
| | GAGGACTTAAGGACATCCTGCCAAGCCACAACAGCAAAAGA | |
| | AAGTACGGCAGCGCCATTGAGGACCTGCTGTTTGACAAGGT | |
| | GGTCACCTCCGGCCTGGGCACAGTGGATGAGGACTACAAGA | |
| | GATGCACCGGCGGCTATGACATTGCCGACCTGGTGTGTGCCC | |
| | AGTACTACAATGGCATCATGGTGCTGCCTGGAGTGGCCAACG | |
| | CCGACAAAATGACCATGTACACCGCCTCCCTGGCTGGAGGC | |
| | ATCACACTGGGAGCCCTGGGGGGAGGAGCAGTGGCCATCCC | |
| | CTTTGCAGTGGCTGTGCAGGCCAGACTCAACTACGTGGCCCT | |
| | GCAGACAGACGTGCTCAACAAGAACCAGCAGAACCTGGCC | |
| | AATGCCTTCATCCAGGCTATCGGAAACATCACCCAGGCCTTT | |
| | GGAAAAGTGAATGATGCCATCCACCAGACCAGCCAGGGCCT | |
| | GGCCACAGTGGCCAAGGCCCTGGCCAAGGTGCAGGACGTGG | |
| | TCAACACCCAGGGCCAGGCCCTCAGTCACCTCACAGTACAG | |
| | CTCCAGAACAACTTCCAGGCAATCTCCTCCTCCATCAGCGAC | |
| | ATCTACAACAGGCTGGACCCCCCAAGCGCTGATGCCCAGGT | |
| | GGACAGACTGATCACAGGAAGACTCACAGCCCTCAACGCAT | |
| | TTGTGTCCCAGACACTGACCAGGCAGGCAGAGGTCAGGGCC | |
| | AGCAGGCAGCTGGCCAAGGACAAGGTGAATGAGTGCGTGA | |
| | GGAGCCAGAGCCAGAGATTTGGCTTCTGCGGAAACGGCACC | |
| | CACCTGTTCAGCCTGGCCAACGCCGCCCCCAACGGCATGATT | |
| | TTCTTCCACACAGTCCTCCTCCCCACAGCCTACGAAACAGTG | |
| | ACAGCCTGGTCAGGCATCTGTGCCAGCGACGGAGACAGAAC | |
| | CTTTGGCCTGGTGGTGAAGGATGTGCAGCTCACCCTCTTCAG | |
| | AAACCTGGATGACAAGTTCTACCTCACCCCAAGAACCATGTA | |
| | CCAGCCCAGAGTGGCCACAAGCAGCGACTTTGTGCAGATTG | |
| | AGGGCTGTGACGTGCTGTTTGTGAATGCAACAGTGATTGACC | |
| | TCCCAAGCATCATCCCAGATTACATCGACATCAACCAGACAG | |
| | TGCAGGACATCCTGGAGAACTACAGGCCCAACTGGACAGTG | |
| | CCAGAGTTCACCCTGGACATCTTCAACGCCACCTACCTGAAC | |
| | CTGACAGGAGAAATTGACGACCTGGAGTTCAGATCAGAAAA | |
| | ACTTCACAACACCACCGTGGAGCTTGCCATCCTCATTGACAC | |
| | CATTAACAACACACTGGTCAACCTGGAATGGCTGAACAGAAT | |
| | TGAAACCTACGTGAAGTGGCCCTGGTATGTGTGGCTGCTGAT | |
| | TGGACTGGTGGTGGTGTTCTGCATCCCACTGCTGCTGTTCTG | |
| | CTGCTTCAGCACCGGCTGCTGTGGATGCATCGGCTGCTTGGG | |
| | CAGCTGCTGCCACAGCATCTGCAGCAGGAGGCAGTTTGAGT | |
| | ACTACGAGCCCATCGAGAAGGTGCACGTGCACTTCCTGGGC | |
| | ATCATCGCCGGCGTGGTGGTCCTGGTGGTCACAGTGGTGGTG | |
| | GGAGCTGTGATCTGGAGAAAGAAGTGCAGCGGCAGGAAGG | |
| | GCCCAAGCTACAGCCACGCTGCCAGAGATGACTCCACCCAG | |
| | GGCAGCGACAGCAGCCTGATGGCCCCCAAGGTG | |
| 133 | ATGAGATTTGTGATGTCCCCCACTGTCCTGCTGCTGCTCCTGG | MHCIsp- |
| | GAGCCCTGGCAGCCCCCCAGACCTGGGCAGGCAGCTACCCA | HA-M |
| | TACGATGTTCCAGACTACGCTTACCCATATGACGTGCCAGACT | (2-C11 Re |
| | ATGCCTACCCCTACGACGTGCCCGACTACGCAGACCGCTACT | 10276)- |
| | GTGCCATGCAGCACGCCAGCAGCACCAGCTGCATCAATGGC | MITD |
| | ACCAGCACCAACAGCTGCCAGACCTGCTTTGAAAGAGGAGA | |
| | CTTGATTTGGCACCTGGCCAACTGGAACTTCAGCTGGAGCGT | |
| | CATCCTCATCGTGTTCATCACCGTGCTGCAGTATGGAAGACCC | |
| | CAGCTGAGCTGGTTTGTGTACGGCATCAAGATGCTCATCATG | |
| | TGGCTGCTGTGGCCCATCGTGCTGGCCCTGACCATCTTCAAC | |
| | GCCTACAGCGAGTACCAGGTGTCCAGATACGTGATGTTTGGC | |
| | TTCTCTGTGGCTGGAGCTGTCATCACCTTTGCCCTGTGGATG | |
| | ATGTACTTTGTGAGGAGCATCCAGCTGTACAGGAGGACCAAG | |
| | AGCTGGTGGAGCTTCAACCCAGAAACCAATGCCATCCTGTGT | |
| | GTGAATGCCCTGGGCAGGAGCTACGTGCTGCCCCTGGATGGC | |
| | ATCCCCACTGGAGTCACCCTGACCCTGCTGTCTGGAAACCTG | |
| | TATGCTGAGTGCTTCAAGATGGTGGGCGGCCTGACCATCGAG | |
| | CACCTGCCCAAGTACGTGATGATTGCCACCCCCAGCAGCACC | |
| | ATCGTGTACACCCTGGTGGGCAAGCAGCTGAAGGCCACCAC | |
| | AGCCACCGGCTGGGCCTACTATGTGAAGAGCAAGGCTGGAG | |
| | ACTACAGCACAGAGGCCAGGACAGACAACCTGTCTGAACAT | |
| | GAGAAGCTGCTGCACATGGTGTTCCTGGGCATCATCGCCGGC | |
| | GTGGTGGTCCTGGTGGTCACAGTGGTGGTGGGAGCTGTGAT | |
| | CTGGAGAAAGAAGTGCAGCGGCAGGAAGGGCCCAAGCTAC | |
| | AGCCACGCTGCCAGAGATGACTCCACCCAGGGCAGCGACAG | |
| | CAGCCTGATGGCCCCCAAGGTG | |
| 134 | ATGCACCACCACCACCACCACGCCACCCAGGGCCAGAGGGT | His-N |
| | CAACTGGGGCGACGAGCCCAGCAAGAGGAGAGGAAGAAGC | (2-C11 Re |
| | AACAGCAGAGGAAGGAAGAACAATGACATCCCCCTGAGCTT | 10276) |
| | CTACAACCCCATCACCCTGGAGACTGGCAGCAAGTTCTGGA | |
| | ATGTCTGCCCCAGGGACTTTGTGCCCAAGGGCATCGGCAACA | |
| | AGGACCAGCAGATTGGCTACTGGAACAAGCAGGCCCGCTAC | |
| | AGAATTGTGAAGGGCCAGAGGAAGGACCTGCCTGAGAGGTG | |
| | GTTCTTCTACTTCCTGGGCACAGGCCCCCACGCTGATGCCAA | |
| | GTTCAAGGACAAGATTGATGGAGTCTTCTGGGTGGCCAAGG | |
| | ATGGAGCCATGAACAAGCCCACCACCCTGGGCACCAGAGGC | |
| | ACCAACAATGAGAGCAAGCCCCTGAGATTTGATGGGAAGAT | |
| | CCCCCCCCAGTTCCAGCTGGAGGTGAACCAGAGCAGAAACA | |
| | ACAGCAGAAGCGGCAGCCAGAGCAGAAGTGCCTCCAGAAA | |
| | CAGAAGCCAGAGCAGAGGAAGACAGCAGAGCAACAACCAG | |
| | AACACCAACGTGGAGGACACCATCGTGGCTGTGCTGCAGAA | |
| | GCTGGGCGTGACAGACAAGCAGAGGAGCAGAAGCAAGAGC | |
| | AGAGAAAGAAGCGGCAGCAACAGCAGGGACACCACCCCCA | |
| | AGAATGCCAACAAGCACAGCTGGAAGAAGACAGCTGGCAA | |
| | GGGAGATGTGACCAACTTCTATGGAGCCAGGAGTGCCAGCG | |
| | CCAACTTCGGGGACAGTGACCTGGTGGCCAATGGAAATGCC | |
| | GCCAAGTGCTACCCCCAGATTGCTGAGTGTGTGCCCAGCGTG | |
| | TCCTCCATGCTGTTTGGAAGCCAGTGGTCAGCAGAGGATGCT | |
| | GGGGACCAGGTGAAGGTGACCCTGACCCACACCTACTACCT | |
| | GCCCAAGGATGATGCCAAGACCAGCCAGTTCCTGGGCCAGA | |
| | TTGATGCCTACAAGAGGCCCAGCCAGGTGGTGAAGGAGCAG | |
| | AGGCAGAGGAAGAGCAGAAGCAAGTCTGCTGACAAGAAGC | |
| | CAGAGGAGCTGTCTGTGACCCTGGTGGAGGCCTACACAGAC | |
| | GTGTTTGATGACACCCAGGTGGAGATGATTGATGAGGTGACC | |
| | AAC | |
| 135 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACTAC | Flag-SII |
| | AAGGATGATGATGACAAGGACTACAAAGACGACGACGACAA | (2-C11 Re |
| | GGACTACAAGGATGACGATGACAAGTGTGAGCCCATCATCAC | 10276)- |
| | CTACTTCAACATCGGGGTGTGCAAGAATGGGGCCCTGGTCTT | MITD |
| | CATCAATGTGACCCACAGCGATGGAGATGTGCAGCCCATCAG | |
| | CACAGGAAATGTGACCATCCCCACCAACTTCACCATCTCTGT | |
| | GCAGGTGGAGTACATCCAGGTGTACACCACCCCTGTGTCCAT | |
| | CGACTGCAGCCGCTACGTGTGCAATGGAAACCCCAGGTGCA | |
| | ACAAGCTGCTGACCCAGTACTTCTCTGCCTGCCAGACCATCG | |
| | AGCAGGCCCTGGCCATGGGAGCCCGGCTGGAGAACATGGAG | |
| | GTGGACAGCATGCTGTTTGTGTCTGAAAATGCCCTGAAGCTG | |
| | GCCTCTGTGGAGGCCTTCAACAGCAGTGAGCACCTGGACCC | |
| | CATCTACAAGGAGTGGCCCAACATTGGAGGCAGCTGGCTGG | |
| | GGGGCCTGAAGGACATCCTGCCCAGCCACAACAGCAAGAGG | |
| | AACTACAGAAGTGCCATCGAGGACCTGCTCTTTGACAAGGT | |
| | GGTGACCTCTGGGCTGGGCACCGTGGATGATGACTACAAGA | |
| | GGTGCACAGGAGGCTATGACATTGCAGACCTGGTGTGTGCCC | |
| | AGTACTACCATGGCATCATGGTGCTGCCTGGAGTGGCCAATG | |
| | ATGACAAGATGACCATGTACACAGCCTCCCTGGCTGGAGGCA | |
| | TCACCCTGGGGGCCCTGGGCGGAGGAGCCGTGGCCATCCCC | |
| | TTTGCTGTGGCTGTGCAGGCCAGGCTCAACTACGTGGCCCTG | |
| | CAGACAGATGTGCTCAACAAGAACCAGCAGATCCTGGCCAA | |
| | CGCCTTCAACCAGGCCATTGGAAACATCACCCAGGCCTTTGG | |
| | GAAGGTGAATGATGCCATCCACCAGACCAGCAAGGGCCTGG | |
| | CCACCGTGGCCAAGGCCCTGGCCAAGGTGCAGGATGTGGTC | |
| | AACACCCAGGGCCAGGCCCTGAGCCACCTCACTGTCCAGCT | |
| | GCAGAACAACTTCCAGGCCATCAGCAGCAGCATCTCTGACAT | |
| | CTACAACAGGCTGGATGAGCTGTCTGCTGATGCCCAGGTGGA | |
| | CAGACTCATCACCGGGAGGCTGACAGCCCTGAATGCCTTTGT | |
| | CAGCCAGACCCTGACCAGGCAGGCAGAGGTGCGGGCCTCCC | |
| | GGCAGCTGGCCAAGGACAAGGTGAATGAGTGTGTGAGGAGC | |
| | CAGAGCCAGAGGTTTGGCTTCTGTGGAAATGGCACCCACCT | |
| | CTTCTCCCTGGCCAATGCTGCCCCCAATGGCATGATCTTCTTC | |
| | CACACCGTGCTGCTGCCCACCGCCTATGAAACAGTGACAGC | |
| | CTGGAGCGGCATCTGTGCCTCTGATGGGGACCACACCTTCGG | |
| | CCTGGTGGTGAAGGATGTCCAGCTGACCCTCTTCAGAAACCT | |
| | GGATGACAAGTTCTACCTGACCCCCAGGACCATGTACCAGCC | |
| | CCGGGTGGCCACCAGCAGCGACTTTGTGCAGATTGAGGGCT | |
| | GTGATGCCCTGTTTGTGAATGCCACTGTCATCGAGCTGCCCA | |
| | GCATCATCCCAGACTACATTGACATCAACCAGACCGTGCAGG | |
| | ACATCCTGAAGAACTACAGGCCCAACTGGACAGTTCCTGAG | |
| | CTGACCCTGGACATCTTCAACAGCACCTACCTGAACCTGACA | |
| | GGAGAAATCAATGACCTGGAGTTCAGAAGTGAGAAGCTGCA | |
| | CAACACCACAGTGGAGCTGGCTGTGCTGATCGACAACATCA | |
| | ACAACACCCTGGTCAACCTGGAGTGGCTGAACAGAATTGAA | |
| | ACCTACGTGAAGTGGCCCTGGTATGTTTGGCTGCTCATCGGC | |
| | CTGGTGCTGGTGTTCTGCATCCCCCTGCTCATGTTCTGCTGCC | |
| | TGAGCACCGGCTGCTGCGGCTGCTTCGGCTGCCTGGGCAGC | |
| | TGCTGCCACAGCCTGTTCTCCAGAAGACACTTTGAGAACTAC | |
| | GAGCCCATCGAGAAGGTGCACATCCACTTCCTGGGCATCATC | |
| | GCCGGCGTGGTGGTCCTGGTGGTCACAGTGGTGGTGGGAGC | |
| | TGTGATCTGGAGAAAGAAGTGCAGCGGCAGGAAGGGCCCA | |
| | AGCTACAGCCACGCTGCCAGAGATGACTCCACCCAGGGCAG | |
| | CGACAGCAGCCTGATGGCCCCCAAGGTG | |
| 136 | ATGGAGAGATACTGTGCCATGCAGAACACAGGCAGCCAGTG | M(M2) |
| | CATCAATGGAACAGACAGCAGCTGCAGCACCTGCTTTGAAA | |
| | GAGGAGGCCTGATCTGGCACCTGGCCAACTGGAACTTCAGC | |
| | TGGAGTGTGATCCTGATTGTCTTCATTACAGTGCTGAAGTACG | |
| | GCAGGCCCCAGTTCAGCTGGCTGGTGTACGGCATCAAGATGC | |
| | TCATCATGTGGCTGCTGTGGCCCATTGTGTTGGCCCTCACCAT | |
| | CTTCAATGCCTACAGCGAGTACCAGGTGTCCAGATACGTGAT | |
| | GTTCGGCTTCTCAGTGGCAGGAGCCGTGGTGACCTTTGCCCT | |
| | CTGGATGATGTACTTTGTGAGGTCCATCCAGCTCTACAGAAG | |
| | AACAAAGAGCTGGTGGAGCTTCAACCCAGAAACCAACGCCA | |
| | TCCTGTGTGTCAATGCCCTGGGCAGATCCTATGTGCTGCCCCT | |
| | GGATGGCACCCCCACAGGCGTCACCCTCACCCTCCTGAGCG | |
| | GCAACCTGTACGCCGAGGGCTTCAAGATGGCCGGCGGCCTG | |
| | ACAATCGAGCACCTGCCCAAGTATGTGATGATTGCCACCCCC | |
| | AGCAGGACAATAGTCTACACCCTGGTGGGAAAACAGCTGAA | |
| | GGCTACCACAGCCACAGGCTGGGCCTACTACGTCAAGAGCA | |
| | AGGCCGGCGACTACAGCACAGAGGCCAGGACCGACAACCT | |
| | GTCCGAACATGAAAAACTCCTGCACATGGTC | |
| 137 | ATGGAGAGGTACTGCGCCATGCAGAACACAGGCTCCCAGTG | M(M3) |
| | CATCAACGGAACAGACTCCTCCTGCAGCACCTGCTTCGAGA | |
| | GAGGAGGCCTCATTTGGCACCTGGCCAACTGGAACTTCAGC | |
| | TGGTCAGTCATTCTGATAGTCTTCATAACAGTGCTGAAGTACG | |
| | GCAGGCCCCAGTTCTCCTGGCTCGTGTATGGCATCAAGATGC | |
| | TGATCATGTGGCTGCTGTGGCCCATCGTCCTGGCCCTGACCAT | |
| | CTTCAACGCCTACTCAGAGTACCAGGTCAGCAGGTACGTGAT | |
| | GTTCGGCTTCTCCGTGGCCGGAGCAGTGGTGACCTTCGCCCT | |
| | GTGGATGATGTACTTCGTGAGGAGCATCCAACTGTACAGGAG | |
| | GACCAAAAGCTGGTGGTCCTTCAACCCAGAAACCAACGCCA | |
| | TCCTCTGCGTGAACGCCCTGGGCAGGTCCTACGTCCTCCCCC | |
| | TGGACGGCACCCCCACCGGGGTCACCCTCACCCTCCTGTCA | |
| | GGGAACCTGTACGCTGAGGGCTTCAAGATGGCTGGAGGCCT | |
| | GACAATTGAACACCTGCCCAAGTACGTCATGATCGCAACACC | |
| | CTCCAGAACCATCGTCTACACCCTGGTGGGCAAGCAGCTGA | |
| | AGGCCACCACCGCCACCGGCTGGGCCTACTACGTCAAGTCC | |
| | AAGGCCGGCGACTACAGCACCGAGGCCAGGACCGACAACCT | |
| | CTCAGAGCACGAGAAGCTGCTGCACATGGTG | |
| 138 | ATGGAGAGGTACTGTGCCATGCAGAACACCGGTTCCCAGTGC | M(M6) |
| | ATCAACGGCACAGACTCCTCCTGCTCCACCTGCTTCGAGAGA | |
| | GGAGGCCTGATCTGGCACCTGGCAAACTGGAACTTCAGCTG | |
| | GAGCGTGATCCTGATAGTGTTCATAACCGTCCTGAAGTACGG | |
| | CAGACCACAGTTCTCATGGCTTGTCTATGGCATCAAGATGCT | |
| | GATTATGTGGCTGCTTTGGCCTATCGTCCTGGCCCTGACCATC | |
| | TTCAACGCCTACTCTGAGTACCAGGTGTCAAGGTATGTCATG | |
| | TTCGGCTTCTCAGTGGCTGGAGCTGTGGTGACCTTTGCTCTG | |
| | TGGATGATGTACTTCGTGAGGTCCATCCAGCTGTACAGGAGG | |
| | ACAAAGTCATGGTGGTCCTTCAACCCAGAAACCAATGCCATC | |
| | CTGTGCGTCAACGCACTGGGCAGAAGCTACGTCCTACCACTG | |
| | GACGGCACTCCTACAGGAGTGACCCTGACCCTGCTGTCAGG | |
| | CAATCTGTACGCAGAGGGGTTCAAGATGGCCGGTGGCCTGA | |
| | CCATCGAGCATCTGCCTAAGTACGTGATGATCGCCACCCCTA | |
| | GCAGGACAATCGTGTACACCCTGGTGGGAAAGCAGCTAAAG | |
| | GCGACCACAGCCACAGGCTGGGCCTACTACGTGAAGTCCAA | |
| | GGCAGGGGACTATTCAACCGAGGCCAGGACCGACAACCTGT | |
| | CAGAGCACGAGAAGCTGCTGCACATGGTC | |
| 139 | ATGGAGAGATACTGTGCCATGCAGAACACAGGCAGCCAGTG | M_d(M2) |
| | CATCAATGGAACAGACAGCAGCTGCAGCACCTGCTTTGAAA | |
| | GAGGAGGCCTGATCTGGCACCTGGCCAACTGGAACTTCAGC | |
| | TGGAGTGTGATCCTGATTGTCTTCATTACAGTGCTGAAGTACG | |
| | GCAGGCCCCAGTTCAGCTGGCTGGTGTACGGCATCATTGTGT | |
| | TGGCCCTCACCATCTTCAATGCCTACAGCGAGTACCAGGTGT | |
| | CCAGATACGTGATGTTCGGCTTCTCAGTGGCAGGAGCCGTGG | |
| | TGACCTTTGCCCTCTGGATGATGTACTTTGTGAGGTCCATCCA | |
| | GCTCTACAGAAGAACAAAGAGCTGGTGGAGCTTCAACCCAG | |
| | AAACCAACGCCATCCTGTGTGTCAATGCCCTGGGCAGATCCT | |
| | ATGTGCTGCCCCTGGATGGCACCCCCACAGGCGTCACCCTCA | |
| | CCCTCCTGAGCGGCAACCTGTACGCCGAGGGCTTCAAGATG | |
| | GCCGGCGGCCTGACAATCGAGCACCTGCCCAAGTATGTGATG | |
| | ATTGCCACCCCCAGCAGGACAATAGTCTACACCCTGGTGGGA | |
| | AAACAGCTGAAGGCTACCACAGCCACAGGCTGGGCCTACTA | |
| | CGTCAAGAGCAAGGCCGGCGACTACAGCACAGAGGCCAGG | |
| | ACCGACAACCTGTCCGAACATGAAAAACTCCTGCACATGGT | |
| | C | |
| 140 | ATGGAGAGGTACTGCGCCATGCAGAACACAGGCTCCCAGTG | M_d(M3) |
| | CATCAACGGAACAGACTCCTCCTGCAGCACCTGCTTCGAGA | |
| | GAGGAGGCCTCATTTGGCACCTGGCCAACTGGAACTTCAGC | |
| | TGGTCAGTCATTCTGATAGTCTTCATAACAGTGCTGAAGTACG | |
| | GCAGGCCCCAGTTCTCCTGGCTCGTGTATGGCATCATCGTCCT | |
| | GGCCCTGACCATCTTCAACGCCTACTCAGAGTACCAGGTCAG | |
| | CAGGTACGTGATGTTCGGCTTCTCCGTGGCCGGAGCAGTGGT | |
| | GACCTTCGCCCTGTGGATGATGTACTTCGTGAGGAGCATCCA | |
| | ACTGTACAGGAGGACCAAAAGCTGGTGGTCCTTCAACCCAG | |
| | AAACCAACGCCATCCTCTGCGTGAACGCCCTGGGCAGGTCC | |
| | TACGTCCTCCCCCTGGACGGCACCCCCACCGGGGTCACCCTC | |
| | ACCCTCCTGTCAGGGAACCTGTACGCTGAGGGCTTCAAGATG | |
| | GCTGGAGGCCTGACAATTGAACACCTGCCCAAGTACGTCATG | |
| | ATCGCAACACCCTCCAGAACCATCGTCTACACCCTGGTGGGC | |
| | AAGCAGCTGAAGGCCACCACCGCCACCGGCTGGGCCTACTA | |
| | CGTCAAGTCCAAGGCCGGCGACTACAGCACCGAGGCCAGGA | |
| | CCGACAACCTCTCAGAGCACGAGAAGCTGCTGCACATGGTG | |
| 141 | ATGGAGAGGTACTGTGCCATGCAGAACACCGGTTCCCAGTGC | M_d(M6) |
| | ATCAACGGCACAGACTCCTCCTGCTCCACCTGCTTCGAGAGA | |
| | GGAGGCCTGATCTGGCACCTGGCAAACTGGAACTTCAGCTG | |
| | GAGCGTGATCCTGATAGTGTTCATAACCGTCCTGAAGTACGG | |
| | CAGACCACAGTTCTCATGGCTTGTCTATGGCATCATCGTCCTG | |
| | GCCCTGACCATCTTCAACGCCTACTCTGAGTACCAGGTGTCA | |
| | AGGTATGTCATGTTCGGCTTCTCAGTGGCTGGAGCTGTGGTG | |
| | ACCTTTGCTCTGTGGATGATGTACTTCGTGAGGTCCATCCAGC | |
| | TGTACAGGAGGACAAAGTCATGGTGGTCCTTCAACCCAGAA | |
| | ACCAATGCCATCCTGTGCGTCAACGCACTGGGCAGAAGCTAC | |
| | GTCCTACCACTGGACGGCACTCCTACAGGAGTGACCCTGACC | |
| | CTGCTGTCAGGCAATCTGTACGCAGAGGGGTTCAAGATGGCC | |
| | GGTGGCCTGACCATCGAGCATCTGCCTAAGTACGTGATGATC | |
| | GCCACCCCTAGCAGGACAATCGTGTACACCCTGGTGGGAAA | |
| | GCAGCTAAAGGCGACCACAGCCACAGGCTGGGCCTACTACG | |
| | TGAAGTCCAAGGCAGGGGACTATTCAACCGAGGCCAGGACC | |
| | GACAACCTGTCAGAGCACGAGAAGCTGCTGCACATGGTC | |
| 142 | ATGAGATTTGTGATGTCCCCCACTGTCCTGCTGCTGCTCCTGG | MHCIsp- |
| | GAGCCCTGGCAGCCCCCCAGACCTGGGCAGGCAGCGAGAG | M(M2) |
| | ATACTGTGCCATGCAGAACACAGGCAGCCAGTGCATCAATGG | |
| | AACAGACAGCAGCTGCAGCACCTGCTTTGAAAGAGGAGGCC | |
| | TGATCTGGCACCTGGCCAACTGGAACTTCAGCTGGAGTGTG | |
| | ATCCTGATTGTCTTCATTACAGTGCTGAAGTACGGCAGGCCC | |
| | CAGTTCAGCTGGCTGGTGTACGGCATCAAGATGCTCATCATG | |
| | TGGCTGCTGTGGCCCATTGTGTTGGCCCTCACCATCTTCAATG | |
| | CCTACAGCGAGTACCAGGTGTCCAGATACGTGATGTTCGGCT | |
| | TCTCAGTGGCAGGAGCCGTGGTGACCTTTGCCCTCTGGATGA | |
| | TGTACTTTGTGAGGTCCATCCAGCTCTACAGAAGAACAAAGA | |
| | GCTGGTGGAGCTTCAACCCAGAAACCAACGCCATCCTGTGT | |
| | GTCAATGCCCTGGGCAGATCCTATGTGCTGCCCCTGGATGGC | |
| | ACCCCCACAGGCGTCACCCTCACCCTCCTGAGCGGCAACCT | |
| | GTACGCCGAGGGCTTCAAGATGGCCGGCGGCCTGACAATCG | |
| | AGCACCTGCCCAAGTATGTGATGATTGCCACCCCCAGCAGGA | |
| | CAATAGTCTACACCCTGGTGGGAAAACAGCTGAAGGCTACC | |
| | ACAGCCACAGGCTGGGCCTACTACGTCAAGAGCAAGGCCGG | |
| | CGACTACAGCACAGAGGCCAGGACCGACAACCTGTCCGAAC | |
| | ATGAAAAACTCCTGCACATGGTC | |
| 143 | ATGAGATTTGTGATGAGCCCCACTGTGCTGCTGCTGCTGCTG | MHCIsp- |
| | GGAGCCCTGGCAGCCCCCCAGACCTGGGCTGGCTCCGAGAG | M(M3) |
| | GTACTGCGCCATGCAGAACACAGGCTCCCAGTGCATCAACG | |
| | GAACAGACTCCTCCTGCAGCACCTGCTTCGAGAGAGGAGGC | |
| | CTCATTTGGCACCTGGCCAACTGGAACTTCAGCTGGTCAGTC | |
| | ATTCTGATAGTCTTCATAACAGTGCTGAAGTACGGCAGGCCC | |
| | CAGTTCTCCTGGCTCGTGTATGGCATCAAGATGCTGATCATGT | |
| | GGCTGCTGTGGCCCATCGTCCTGGCCCTGACCATCTTCAACG | |
| | CCTACTCAGAGTACCAGGTCAGCAGGTACGTGATGTTCGGCT | |
| | TCTCCGTGGCCGGAGCAGTGGTGACCTTCGCCCTGTGGATGA | |
| | TGTACTTCGTGAGGAGCATCCAACTGTACAGGAGGACCAAA | |
| | AGCTGGTGGTCCTTCAACCCAGAAACCAACGCCATCCTCTGC | |
| | GTGAACGCCCTGGGCAGGTCCTACGTCCTCCCCCTGGACGG | |
| | CACCCCCACCGGGGTCACCCTCACCCTCCTGTCAGGGAACCT | |
| | GTACGCTGAGGGCTTCAAGATGGCTGGAGGCCTGACAATTG | |
| | AACACCTGCCCAAGTACGTCATGATCGCAACACCCTCCAGAA | |
| | CCATCGTCTACACCCTGGTGGGCAAGCAGCTGAAGGCCACC | |
| | ACCGCCACCGGCTGGGCCTACTACGTCAAGTCCAAGGCCGG | |
| | CGACTACAGCACCGAGGCCAGGACCGACAACCTCTCAGAGC | |
| | ACGAGAAGCTGCTGCACATGGTG | |
| 144 | ATGAGATTCGTGATGTCCCCTACCGTACTACTACTCCTACTTG | MHCIsp- |
| | GCGCACTAGCAGCTCCTCAAACTTGGGCCGGATCCGAGAGG | M(M6) |
| | TACTGTGCCATGCAGAACACCGGTTCCCAGTGCATCAACGGC | |
| | ACAGACTCCTCCTGCTCCACCTGCTTCGAGAGAGGAGGCCT | |
| | GATCTGGCACCTGGCAAACTGGAACTTCAGCTGGAGCGTGA | |
| | TCCTGATAGTGTTCATAACCGTCCTGAAGTACGGCAGACCAC | |
| | AGTTCTCATGGCTTGTCTATGGCATCAAGATGCTGATTATGTG | |
| | GCTGCTTTGGCCTATCGTCCTGGCCCTGACCATCTTCAACGCC | |
| | TACTCTGAGTACCAGGTGTCAAGGTATGTCATGTTCGGCTTCT | |
| | CAGTGGCTGGAGCTGTGGTGACCTTTGCTCTGTGGATGATGT | |
| | ACTTCGTGAGGTCCATCCAGCTGTACAGGAGGACAAAGTCAT | |
| | GGTGGTCCTTCAACCCAGAAACCAATGCCATCCTGTGCGTCA | |
| | ACGCACTGGGCAGAAGCTACGTCCTACCACTGGACGGCACT | |
| | CCTACAGGAGTGACCCTGACCCTGCTGTCAGGCAATCTGTAC | |
| | GCAGAGGGGTTCAAGATGGCCGGTGGCCTGACCATCGAGCA | |
| | TCTGCCTAAGTACGTGATGATCGCCACCCCTAGCAGGACAAT | |
| | CGTGTACACCCTGGTGGGAAAGCAGCTAAAGGCGACCACAG | |
| | CCACAGGCTGGGCCTACTACGTGAAGTCCAAGGCAGGGGAC | |
| | TATTCAACCGAGGCCAGGACCGACAACCTGTCAGAGCACGA | |
| | GAAGCTGCTGCACATGGTC | |
| 145 | ATGAGATTTGTGATGTCCCCCACTGTCCTGCTGCTGCTCCTGG | MHCIsp- |
| | GAGCCCTGGCAGCCCCCCAGACCTGGGCAGGCAGCGAGAG | M(M2)- |
| | ATACTGTGCCATGCAGAACACAGGCAGCCAGTGCATCAATGG | MITD |
| | AACAGACAGCAGCTGCAGCACCTGCTTTGAAAGAGGAGGCC | |
| | TGATCTGGCACCTGGCCAACTGGAACTTCAGCTGGAGTGTG | |
| | ATCCTGATTGTCTTCATTACAGTGCTGAAGTACGGCAGGCCC | |
| | CAGTTCAGCTGGCTGGTGTACGGCATCAAGATGCTCATCATG | |
| | TGGCTGCTGTGGCCCATTGTGTTGGCCCTCACCATCTTCAATG | |
| | CCTACAGCGAGTACCAGGTGTCCAGATACGTGATGTTCGGCT | |
| | TCTCAGTGGCAGGAGCCGTGGTGACCTTTGCCCTCTGGATGA | |
| | TGTACTTTGTGAGGTCCATCCAGCTCTACAGAAGAACAAAGA | |
| | GCTGGTGGAGCTTCAACCCAGAAACCAACGCCATCCTGTGT | |
| | GTCAATGCCCTGGGCAGATCCTATGTGCTGCCCCTGGATGGC | |
| | ACCCCCACAGGCGTCACCCTCACCCTCCTGAGCGGCAACCT | |
| | GTACGCCGAGGGCTTCAAGATGGCCGGCGGCCTGACAATCG | |
| | AGCACCTGCCCAAGTATGTGATGATTGCCACCCCCAGCAGGA | |
| | CAATAGTCTACACCCTGGTGGGAAAACAGCTGAAGGCTACC | |
| | ACAGCCACAGGCTGGGCCTACTACGTCAAGAGCAAGGCCGG | |
| | CGACTACAGCACAGAGGCCAGGACCGACAACCTGTCCGAAC | |
| | ATGAAAAACTCCTGCACATGGTCTTCCTGGGCATCATCGCTG | |
| | GCGTGGTGGTCCTGGTGGTGACTGTGGTGGTCGGAGCCGTC | |
| | ATCTGGAGGAAGAAGTGCAGCGGCAGAAAGGGCCCAAGCTA | |
| | CAGCCACGCCGCCAGAGATGACAGCACCCAGGGCAGCGACA | |
| | GCAGCCTCATGGCCCCCAAGGTC | |
| 146 | ATGAGATTTGTGATGAGCCCCACTGTGCTGCTGCTGCTGCTG | MHCIsp- |
| | GGAGCCCTGGCAGCCCCCCAGACCTGGGCTGGCTCCGAGAG | M(M3)- |
| | GTACTGCGCCATGCAGAACACAGGCTCCCAGTGCATCAACG | MITD |
| | GAACAGACTCCTCCTGCAGCACCTGCTTCGAGAGAGGAGGC | |
| | CTCATTTGGCACCTGGCCAACTGGAACTTCAGCTGGTCAGTC | |
| | ATTCTGATAGTCTTCATAACAGTGCTGAAGTACGGCAGGCCC | |
| | CAGTTCTCCTGGCTCGTGTATGGCATCAAGATGCTGATCATGT | |
| | GGCTGCTGTGGCCCATCGTCCTGGCCCTGACCATCTTCAACG | |
| | CCTACTCAGAGTACCAGGTCAGCAGGTACGTGATGTTCGGCT | |
| | TCTCCGTGGCCGGAGCAGTGGTGACCTTCGCCCTGTGGATGA | |
| | TGTACTTCGTGAGGAGCATCCAACTGTACAGGAGGACCAAA | |
| | AGCTGGTGGTCCTTCAACCCAGAAACCAACGCCATCCTCTGC | |
| | GTGAACGCCCTGGGCAGGTCCTACGTCCTCCCCCTGGACGG | |
| | CACCCCCACCGGGGTCACCCTCACCCTCCTGTCAGGGAACCT | |
| | GTACGCTGAGGGCTTCAAGATGGCTGGAGGCCTGACAATTG | |
| | AACACCTGCCCAAGTACGTCATGATCGCAACACCCTCCAGAA | |
| | CCATCGTCTACACCCTGGTGGGCAAGCAGCTGAAGGCCACC | |
| | ACCGCCACCGGCTGGGCCTACTACGTCAAGTCCAAGGCCGG | |
| | CGACTACAGCACCGAGGCCAGGACCGACAACCTCTCAGAGC | |
| | ACGAGAAGCTGCTGCACATGGTGTTCCTGGGCATCATCGCAG | |
| | GAGTGGTGGTCCTGGTGGTCACAGTCGTGGTGGGAGCAGTG | |
| | ATCTGGAGGAAGAAGTGCTCAGGAAGGAAGGGCCCATCCTA | |
| | CTCCCACGCCGCCAGGGACGACTCAACCCAGGGCTCAGACA | |
| | GCTCCCTGATGGCCCCCAAGGTG | |
| 147 | ATGAGATTCGTGATGTCCCCTACCGTACTACTACTCCTACTTG | MHCIsp- |
| | GCGCACTAGCAGCTCCTCAAACTTGGGCCGGATCCGAGAGG | M(M6)- |
| | TACTGTGCCATGCAGAACACCGGTTCCCAGTGCATCAACGGC | MITD |
| | ACAGACTCCTCCTGCTCCACCTGCTTCGAGAGAGGAGGCCT | |
| | GATCTGGCACCTGGCAAACTGGAACTTCAGCTGGAGCGTGA | |
| | TCCTGATAGTGTTCATAACCGTCCTGAAGTACGGCAGACCAC | |
| | AGTTCTCATGGCTTGTCTATGGCATCAAGATGCTGATTATGTG | |
| | GCTGCTTTGGCCTATCGTCCTGGCCCTGACCATCTTCAACGCC | |
| | TACTCTGAGTACCAGGTGTCAAGGTATGTCATGTTCGGCTTCT | |
| | CAGTGGCTGGAGCTGTGGTGACCTTTGCTCTGTGGATGATGT | |
| | ACTTCGTGAGGTCCATCCAGCTGTACAGGAGGACAAAGTCAT | |
| | GGTGGTCCTTCAACCCAGAAACCAATGCCATCCTGTGCGTCA | |
| | ACGCACTGGGCAGAAGCTACGTCCTACCACTGGACGGCACT | |
| | CCTACAGGAGTGACCCTGACCCTGCTGTCAGGCAATCTGTAC | |
| | GCAGAGGGGTTCAAGATGGCCGGTGGCCTGACCATCGAGCA | |
| | TCTGCCTAAGTACGTGATGATCGCCACCCCTAGCAGGACAAT | |
| | CGTGTACACCCTGGTGGGAAAGCAGCTAAAGGCGACCACAG | |
| | CCACAGGCTGGGCCTACTACGTGAAGTCCAAGGCAGGGGAC | |
| | TATTCAACCGAGGCCAGGACCGACAACCTGTCAGAGCACGA | |
| | GAAGCTGCTGCACATGGTCTTCCTGGGCATCATCGCAGGAGT | |
| | GGTGGTGCTGGTGGTGACCGTGGTGGTGGGGGCTGTAATCTG | |
| | GAGGAAGAAGTGCTCAGGGAGAAAGGGCCCAAGCTACTCTC | |
| | ACGCCGCCAGGGATGACTCCACACAGGGCTCAGACTCCTCA | |
| | CTGATGGCTCCAAAGGTC | |
| 148 | ATGGCCACACAGGGCCAGAGGGTGAACTGGGGCGACGAGC | N(N2) |
| | CATCCAAGAGGAGGGGAAGGAGCAACAGCAGAGGAAGGAA | |
| | GAACAACACCATCCCCCTGTCCTTCTTCAACCCAATTCAGCT | |
| | AGAGCCAGGCAGCAAGTTCTGGTCAGTGTGCCCCAGAGACT | |
| | TCGTGCCCAAGGGCATCGGAAACAAGGACCAGCAGATCGGC | |
| | TACTGGAACAGACAGGAGAGATACAGAATTGTGAAAGGCCA | |
| | GAGAAAGGAGCTGCCAGAGAGGTGGTTCTTCTACTTCCTGG | |
| | GCACCGGCCCACAGGCAGACGCCAAGTTCAAGGACAAGATC | |
| | GATGGAGTGTTCTGGGTGGCCAAGGACGGCGCCATGAACAA | |
| | GCCCACCACACTGGGCACAAGAGGAACAAACAATGAGAGC | |
| | AAGCCACTGAAGTTTGATGGCAAGATCCCACCCCAGTTCCAG | |
| | CTGGAGGTGAACAGGAGCAGAAACAACAGCAGAAGCGGCA | |
| | GCCAGAGCAGAAGTGTGAGCAGAAACAGAAGCCAGAGCAG | |
| | AGGAAGACAGCAGAGCAACAACCAGAACAACGTGGAGGAC | |
| | ACCATCGTGGCCGTGCTGCAGAAGCTGGGGGTCACAGAAAA | |
| | GCAGAGGAGCAGAAGCAAGAGCAGGGACAGAGGAGACAGC | |
| | AAGCCAAGAGACACCACCCCCAACAACGCCAACAAGCACA | |
| | CCTGGAAGAAGACAGCCGGCAAGGGAGATGTGACCAACTTC | |
| | TACGGCGCCAGAAGCGCCAGCGCCAACTTCGGAGACTCAGA | |
| | CCTGGTGGCCAATGGAAACGCAGCCAAGAGCTACCCCCAGA | |
| | TCGCAGAGTGTGTGCCCTCTGTCTCCAGCATGCTGTTTGGCA | |
| | GCCAGTGGAGCGCCGAGGACGACGGTGACCAGGTGAAGGT | |
| | GACCCTGACACACACATACTACCTGCCCAAAGATGACGCCAA | |
| | GACCAGCCAGTTCCTGGAGCAGATTGATGCCTACAAGAGGC | |
| | CCAGCCAGGTGGCCAAGGACCAGAGACAGAGGAAGAGCAG | |
| | GTCCAAGAGCGCCGAGAAGAAGCCAGAAGAATTGAGTGTCA | |
| | CCCTGGTGGAGGCCTACACAGACGTGTTTGATGACACCCAG | |
| | GTGGAGATGATTGATGAGGTGACCAAC | |
| 149 | ATGGCCACCCAGGGCCAGAGAGTGAACTGGGGCGACGAGCC | N(N3) |
| | CTCAAAAAGGAGGGGCAGATCCAACAGCAGAGGCAGGAAG | |
| | AACAACACCATCCCCCTGAGCTTCTTCAACCCCATCCAGCTG | |
| | GAGCCAGGCTCCAAGTTCTGGTCAGTGTGCCCAAGGGACTT | |
| | CGTGCCCAAGGGCATCGGCAACAAGGACCAGCAGATCGGCT | |
| | ACTGGAACAGGCAGGAGAGATACAGAATCGTGAAGGGCCAG | |
| | AGGAAGGAACTGCCAGAAAGGTGGTTCTTCTACTTCCTGGG | |
| | CACCGGCCCCCAGGCTGACGCCAAGTTCAAAGACAAGATCG | |
| | ACGGGGTGTTCTGGGTGGCCAAGGACGGCGCCATGAACAAG | |
| | CCAACAACACTGGGCACCAGAGGAACCAACAACGAGAGCA | |
| | AGCCACTGAAGTTTGACGGCAAGATCCCCCCCCAGTTCCAG | |
| | CTGGAAGTCAACAGGAGCAGGAACAACAGCAGGTCCGGCT | |
| | CACAAAGCAGGAGCGTGTCCAGAAACAGATCCCAGTCAAGA | |
| | GGAAGACAGCAGTCCAACAACCAGAACAACGTGGAGGACA | |
| | CCATAGTGGCCGTGCTGCAGAAGCTGGGAGTCACAGAGAAG | |
| | CAGAGGAGCAGATCCAAGTCCAGGGACAGGGGAGACAGCA | |
| | AGCCCAGGGACACCACACCCAACAACGCCAACAAGCACAC | |
| | CTGGAAGAAGACAGCCGGCAAGGGAGATGTGACCAACTTCT | |
| | ACGGCGCCAGAAGCGCCTCAGCCAACTTCGGAGACTCAGAC | |
| | CTGGTGGCCAACGGAAACGCCGCCAAGAGCTACCCCCAGAT | |
| | CGCCGAATGTGTCCCCTCAGTGTCCTCCATGCTCTTCGGCTC | |
| | ACAGTGGTCAGCAGAGGACGACGGCGACCAGGTGAAGGTG | |
| | ACCCTGACCCACACCTACTACCTGCCCAAGGACGACGCCAA | |
| | GACAAGCCAGTTCCTGGAGCAGATCGACGCCTACAAGAGGC | |
| | CATCCCAGGTGGCCAAGGACCAGAGGCAGAGGAAGAGCAG | |
| | AAGCAAGTCAGCCGAGAAGAAACCAGAGGAGCTGTCAGTC | |
| | ACCCTGGTGGAGGCCTACACCGACGTGTTCGACGACACCCA | |
| | GGTGGAGATGATCGACGAGGTGACCAAC | |
| 150 | ATGGCAACACAAGGACAGAGAGTAAATTGGGGGGATGAGCC | N(N6) |
| | CAGCAAGAGGCGAGGCAGAAGCAACTCAAGAGGGAGAAAA | |
| | AACAATACCATCCCACTGTCATTCTTCAACCCCATTCAACTGG | |
| | AGCCAGGCTCTAAATTCTGGAGTGTATGCCCCAGGGACTTTG | |
| | TGCCCAAGGGCATAGGGAACAAGGACCAGCAAATAGGATAC | |
| | TGGAACCGGCAGGAGAGATACAGAATTGTCAAGGGTCAGAG | |
| | AAAGGAGCTGCCAGAGAGATGGTTCTTCTACTTCCTAGGAAC | |
| | AGGCCCACAGGCAGACGCTAAGTTCAAGGATAAGATCGATG | |
| | GTGTCTTCTGGGTCGCCAAGGATGGTGCAATGAATAAACCAA | |
| | CCACCCTGGGGACCAGGGGGACAAACAATGAGTCCAAGCCC | |
| | CTCAAGTTTGATGGCAAAATCCCCCCACAGTTCCAGCTGGAG | |
| | GTCAACAGGAGCAGGAACAATAGCCGTTCAGGGTCCCAGTC | |
| | CAGATCTGTGTCCAGAAACAGGTCCCAGAGCAGGGGACGGC | |
| | AGCAGAGTAACAACCAGAATAATGTGGAAGACACCATAGTA | |
| | GCAGTGCTCCAGAAACTGGGGGTCACAGAAAAACAGAGGA | |
| | GCAGGTCCAAGTCTAGGGACCGTGGGGACTCTAAGCCAAGG | |
| | GACACCACACCCAACAACGCCAACAAGCACACATGGAAAA | |
| | AAACAGCAGGGAAGGGTGATGTCACCAACTTTTACGGGGCC | |
| | AGGTCAGCCTCTGCAAACTTCGGGGATAGTGACCTGGTGGCC | |
| | AACGGCAATGCTGCTAAATCCTACCCTCAGATTGCTGAGTGC | |
| | GTACCCTCTGTATCCTCTATGCTCTTTGGCTCACAATGGTCTG | |
| | CTGAGGATGATGGTGACCAGGTCAAGGTCACCTTGACCCATA | |
| | CCTACTATCTGCCCAAGGATGATGCAAAAACCAGCCAGTTCC | |
| | TAGAGCAGATAGATGCCTACAAGAGGCCCAGCCAGGTGGCC | |
| | AAGGATCAGAGGCAGAGAAAGAGCAGATCCAAGAGCGCAG | |
| | AAAAGAAACCAGAGGAGTTATCTGTGACCCTGGTGGAGGCC | |
| | TACACAGATGTCTTTGATGATACACAGGTGGAAATGATAGAT | |
| | GAGGTGACTAAC | |
| 151 | ATGGCCACACAGGGCCAGAGGGTGAACTGGGGCGACGAGC | N_d(N2) |
| | CATCCAAGAGGAGGGGAAGGAGCAACAGCAGAGGAAGGAA | |
| | GAACAACACCATCCCCCTGTCCTTCTTCAACCCAATTCAGCT | |
| | AGAGCCAGGCAGCAAGTTCTGGTCAGTGTGCCCCAGAGACT | |
| | TCGTGCCCAAGGGCATCGGAAACAAGGACCAGCAGATCGGC | |
| | TACTGGAACAGACAGGAGAGATACAGAATTGTGAAAGGCCA | |
| | GAGAAAGGAGCTGCCAGAGAGGTGGTTCTTCTACTTCCTGG | |
| | GCACCGGCCCACAGGCAGACGCCAAGTTCAAGGACAAGATC | |
| | GATGGAGTGTTCTGGGTGGCCAAGGACGGCGCCATGAACAA | |
| | GCCCACCACACTGGGCACAAGAGGAACAAACAATGAGAGC | |
| | AAGCCACTGAAGTTTGATGGCAAGATCCCACCCCAGTTCCAG | |
| | CTGGAGGTGAACAGGAGCAGAAGTGTGAGCAGAAACAGAA | |
| | GCCAGAGCAGAGGAAGACAGCAGAGCAACAACCAGAACAA | |
| | CGTGGAGGACACCATCGTGGCCGTGCTGCAGAAGCTGGGGG | |
| | TCACAGAAAAGCAGAGGAGCAGAAGCAAGAGCAGGGACAG | |
| | AGGAGACAGCAAGCCAAGAGACACCACCCCCAACAACGCC | |
| | AACAAGCACACCTGGAAGAAGACAGCCGGCAAGGGAGATG | |
| | TGACCAACTTCTACGGCGCCAGAAGCGCCAGCGCCAACTTC | |
| | GGAGACTCAGACCTGGTGGCCAATGGAAACGCAGCCAAGAG | |
| | CTACCCCCAGATCGCAGAGTGTGTGCCCTCTGTCTCCAGCAT | |
| | GCTGTTTGGCAGCCAGTGGAGCGCCGAGGACGACGGTGACC | |
| | AGGTGAAGGTGACCCTGACACACACATACTACCTGCCCAAA | |
| | GATGACGCCAAGACCAGCCAGTTCCTGGAGCAGATTGATGC | |
| | CTACAAGAGGCCCAGCCAGGTGGCCAAGGACCAGAGACAG | |
| | AGGAAGAGCAGGTCCAAGAGCGCCGAGAAGAAGCCAGAAG | |
| | AATTGAGTGTCACCCTGGTGGAGGCCTACACAGACGTGTTTG | |
| | ATGACACCCAGGTGGAGATGATTGATGAGGTGACCAAC | |
| 152 | ATGGCCACCCAGGGCCAGAGAGTGAACTGGGGCGACGAGCC | N_d(N3) |
| | CTCAAAAAGGAGGGGCAGATCCAACAGCAGAGGCAGGAAG | |
| | AACAACACCATCCCCCTGAGCTTCTTCAACCCCATCCAGCTG | |
| | GAGCCAGGCTCCAAGTTCTGGTCAGTGTGCCCAAGGGACTT | |
| | CGTGCCCAAGGGCATCGGCAACAAGGACCAGCAGATCGGCT | |
| | ACTGGAACAGGCAGGAGAGATACAGAATCGTGAAGGGCCAG | |
| | AGGAAGGAACTGCCAGAAAGGTGGTTCTTCTACTTCCTGGG | |
| | CACCGGCCCCCAGGCTGACGCCAAGTTCAAAGACAAGATCG | |
| | ACGGGGTGTTCTGGGTGGCCAAGGACGGCGCCATGAACAAG | |
| | CCAACAACACTGGGCACCAGAGGAACCAACAACGAGAGCA | |
| | AGCCACTGAAGTTTGACGGCAAGATCCCCCCCCAGTTCCAG | |
| | CTGGAAGTCAACAGGAGCAGGAGCGTGTCCAGAAACAGATC | |
| | CCAGTCAAGAGGAAGACAGCAGTCCAACAACCAGAACAAC | |
| | GTGGAGGACACCATAGTGGCCGTGCTGCAGAAGCTGGGAGT | |
| | CACAGAGAAGCAGAGGAGCAGATCCAAGTCCAGGGACAGG | |
| | GGAGACAGCAAGCCCAGGGACACCACACCCAACAACGCCA | |
| | ACAAGCACACCTGGAAGAAGACAGCCGGCAAGGGAGATGT | |
| | GACCAACTTCTACGGCGCCAGAAGCGCCTCAGCCAACTTCG | |
| | GAGACTCAGACCTGGTGGCCAACGGAAACGCCGCCAAGAG | |
| | CTACCCCCAGATCGCCGAATGTGTCCCCTCAGTGTCCTCCAT | |
| | GCTCTTCGGCTCACAGTGGTCAGCAGAGGACGACGGCGACC | |
| | AGGTGAAGGTGACCCTGACCCACACCTACTACCTGCCCAAG | |
| | GACGACGCCAAGACAAGCCAGTTCCTGGAGCAGATCGACGC | |
| | CTACAAGAGGCCATCCCAGGTGGCCAAGGACCAGAGGCAGA | |
| | GGAAGAGCAGAAGCAAGTCAGCCGAGAAGAAACCAGAGGA | |
| | GCTGTCAGTCACCCTGGTGGAGGCCTACACCGACGTGTTCGA | |
| | CGACACCCAGGTGGAGATGATCGACGAGGTGACCAAC | |
| 153 | ATGGCAACACAAGGACAGAGAGTAAATTGGGGGGATGAGCC | N_d(N6) |
| | CAGCAAGAGGCGAGGCAGAAGCAACTCAAGAGGGAGAAAA | |
| | AACAATACCATCCCACTGTCATTCTTCAACCCCATTCAACTGG | |
| | AGCCAGGCTCTAAATTCTGGAGTGTATGCCCCAGGGACTTTG | |
| | TGCCCAAGGGCATAGGGAACAAGGACCAGCAAATAGGATAC | |
| | TGGAACCGGCAGGAGAGATACAGAATTGTCAAGGGTCAGAG | |
| | AAAGGAGCTGCCAGAGAGATGGTTCTTCTACTTCCTAGGAAC | |
| | AGGCCCACAGGCAGACGCTAAGTTCAAGGATAAGATCGATG | |
| | GTGTCTTCTGGGTCGCCAAGGATGGTGCAATGAATAAACCAA | |
| | CCACCCTGGGGACCAGGGGGACAAACAATGAGTCCAAGCCC | |
| | CTCAAGTTTGATGGCAAAATCCCCCCACAGTTCCAGCTGGAG | |
| | GTCAACAGGAGCAGGTCTGTGTCCAGAAACAGGTCCCAGAG | |
| | CAGGGGACGGCAGCAGAGTAACAACCAGAATAATGTGGAAG | |
| | ACACCATAGTAGCAGTGCTCCAGAAACTGGGGGTCACAGAA | |
| | AAACAGAGGAGCAGGTCCAAGTCTAGGGACCGTGGGGACTC | |
| | TAAGCCAAGGGACACCACACCCAACAACGCCAACAAGCAC | |
| | ACATGGAAAAAAACAGCAGGGAAGGGTGATGTCACCAACTT | |
| | TTACGGGGCCAGGTCAGCCTCTGCAAACTTCGGGGATAGTGA | |
| | CCTGGTGGCCAACGGCAATGCTGCTAAATCCTACCCTCAGAT | |
| | TGCTGAGTGCGTACCCTCTGTATCCTCTATGCTCTTTGGCTCA | |
| | CAATGGTCTGCTGAGGATGATGGTGACCAGGTCAAGGTCACC | |
| | TTGACCCATACCTACTATCTGCCCAAGGATGATGCAAAAACC | |
| | AGCCAGTTCCTAGAGCAGATAGATGCCTACAAGAGGCCCAG | |
| | CCAGGTGGCCAAGGATCAGAGGCAGAGAAAGAGCAGATCC | |
| | AAGAGCGCAGAAAAGAAACCAGAGGAGTTATCTGTGACCCT | |
| | GGTGGAGGCCTACACAGATGTCTTTGATGATACACAGGTGGA | |
| | AATGATAGATGAGGTGACTAAC | |
| 154 | ATGAGATTTGTGATGTCCCCCACTGTCCTGCTGCTGCTCCTGG | MHCIsp- |
| | GAGCCCTGGCAGCCCCCCAGACCTGGGCAGGCAGCGCCACA | N(N2) |
| | CAGGGCCAGAGGGTGAACTGGGGCGACGAGCCATCCAAGA | |
| | GGAGGGGAAGGAGCAACAGCAGAGGAAGGAAGAACAACA | |
| | CCATCCCCCTGTCCTTCTTCAACCCAATTCAGCTAGAGCCAG | |
| | GCAGCAAGTTCTGGTCAGTGTGCCCCAGAGACTTCGTGCCC | |
| | AAGGGCATCGGAAACAAGGACCAGCAGATCGGCTACTGGAA | |
| | CAGACAGGAGAGATACAGAATTGTGAAAGGCCAGAGAAAG | |
| | GAGCTGCCAGAGAGGTGGTTCTTCTACTTCCTGGGCACCGGC | |
| | CCACAGGCAGACGCCAAGTTCAAGGACAAGATCGATGGAGT | |
| | GTTCTGGGTGGCCAAGGACGGCGCCATGAACAAGCCCACCA | |
| | CACTGGGCACAAGAGGAACAAACAATGAGAGCAAGCCACT | |
| | GAAGTTTGATGGCAAGATCCCACCCCAGTTCCAGCTGGAGGT | |
| | GAACAGGAGCAGAAACAACAGCAGAAGCGGCAGCCAGAGC | |
| | AGAAGTGTGAGCAGAAACAGAAGCCAGAGCAGAGGAAGAC | |
| | AGCAGAGCAACAACCAGAACAACGTGGAGGACACCATCGT | |
| | GGCCGTGCTGCAGAAGCTGGGGGTCACAGAAAAGCAGAGG | |
| | AGCAGAAGCAAGAGCAGGGACAGAGGAGACAGCAAGCCAA | |
| | GAGACACCACCCCCAACAACGCCAACAAGCACACCTGGAA | |
| | GAAGACAGCCGGCAAGGGAGATGTGACCAACTTCTACGGCG | |
| | CCAGAAGCGCCAGCGCCAACTTCGGAGACTCAGACCTGGTG | |
| | GCCAATGGAAACGCAGCCAAGAGCTACCCCCAGATCGCAGA | |
| | GTGTGTGCCCTCTGTCTCCAGCATGCTGTTTGGCAGCCAGTG | |
| | GAGCGCCGAGGACGACGGTGACCAGGTGAAGGTGACCCTG | |
| | ACACACACATACTACCTGCCCAAAGATGACGCCAAGACCAG | |
| | CCAGTTCCTGGAGCAGATTGATGCCTACAAGAGGCCCAGCCA | |
| | GGTGGCCAAGGACCAGAGACAGAGGAAGAGCAGGTCCAAG | |
| | AGCGCCGAGAAGAAGCCAGAAGAATTGAGTGTCACCCTGGT | |
| | GGAGGCCTACACAGACGTGTTTGATGACACCCAGGTGGAGA | |
| | TGATTGATGAGGTGACCAAC | |
| 155 | ATGAGATTTGTGATGAGCCCCACTGTGCTGCTGCTGCTGCTG | MHCIsp- |
| | GGAGCCCTGGCAGCCCCCCAGACCTGGGCTGGCTCCGCCAC | N(N3) |
| | CCAGGGCCAGAGAGTGAACTGGGGCGACGAGCCCTCAAAA | |
| | AGGAGGGGCAGATCCAACAGCAGAGGCAGGAAGAACAACA | |
| | CCATCCCCCTGAGCTTCTTCAACCCCATCCAGCTGGAGCCAG | |
| | GCTCCAAGTTCTGGTCAGTGTGCCCAAGGGACTTCGTGCCCA | |
| | AGGGCATCGGCAACAAGGACCAGCAGATCGGCTACTGGAAC | |
| | AGGCAGGAGAGATACAGAATCGTGAAGGGCCAGAGGAAGG | |
| | AACTGCCAGAAAGGTGGTTCTTCTACTTCCTGGGCACCGGCC | |
| | CCCAGGCTGACGCCAAGTTCAAAGACAAGATCGACGGGGTG | |
| | TTCTGGGTGGCCAAGGACGGCGCCATGAACAAGCCAACAAC | |
| | ACTGGGCACCAGAGGAACCAACAACGAGAGCAAGCCACTG | |
| | AAGTTTGACGGCAAGATCCCCCCCCAGTTCCAGCTGGAAGT | |
| | CAACAGGAGCAGGAACAACAGCAGGTCCGGCTCACAAAGC | |
| | AGGAGCGTGTCCAGAAACAGATCCCAGTCAAGAGGAAGAC | |
| | AGCAGTCCAACAACCAGAACAACGTGGAGGACACCATAGTG | |
| | GCCGTGCTGCAGAAGCTGGGAGTCACAGAGAAGCAGAGGA | |
| | GCAGATCCAAGTCCAGGGACAGGGGAGACAGCAAGCCCAG | |
| | GGACACCACACCCAACAACGCCAACAAGCACACCTGGAAG | |
| | AAGACAGCCGGCAAGGGAGATGTGACCAACTTCTACGGCGC | |
| | CAGAAGCGCCTCAGCCAACTTCGGAGACTCAGACCTGGTGG | |
| | CCAACGGAAACGCCGCCAAGAGCTACCCCCAGATCGCCGAA | |
| | TGTGTCCCCTCAGTGTCCTCCATGCTCTTCGGCTCACAGTGG | |
| | TCAGCAGAGGACGACGGCGACCAGGTGAAGGTGACCCTGA | |
| | CCCACACCTACTACCTGCCCAAGGACGACGCCAAGACAAGC | |
| | CAGTTCCTGGAGCAGATCGACGCCTACAAGAGGCCATCCCA | |
| | GGTGGCCAAGGACCAGAGGCAGAGGAAGAGCAGAAGCAAG | |
| | TCAGCCGAGAAGAAACCAGAGGAGCTGTCAGTCACCCTGGT | |
| | GGAGGCCTACACCGACGTGTTCGACGACACCCAGGTGGAGA | |
| | TGATCGACGAGGTGACCAAC | |
| 156 | ATGCGATTCGTCATGTCACCAACCGTTTTACTATTATTACTAGG | MHCIsp- |
| | AGCATTAGCAGCACCGCAAACATGGGCAGGAAGTGCAACAC | N(N6) |
| | AAGGACAGAGAGTAAATTGGGGGGATGAGCCCAGCAAGAG | |
| | GCGAGGCAGAAGCAACTCAAGAGGGAGAAAAAACAATACC | |
| | ATCCCACTGTCATTCTTCAACCCCATTCAACTGGAGCCAGGC | |
| | TCTAAATTCTGGAGTGTATGCCCCAGGGACTTTGTGCCCAAG | |
| | GGCATAGGGAACAAGGACCAGCAAATAGGATACTGGAACCG | |
| | GCAGGAGAGATACAGAATTGTCAAGGGTCAGAGAAAGGAGC | |
| | TGCCAGAGAGATGGTTCTTCTACTTCCTAGGAACAGGCCCAC | |
| | AGGCAGACGCTAAGTTCAAGGATAAGATCGATGGTGTCTTCT | |
| | GGGTCGCCAAGGATGGTGCAATGAATAAACCAACCACCCTG | |
| | GGGACCAGGGGGACAAACAATGAGTCCAAGCCCCTCAAGTT | |
| | TGATGGCAAAATCCCCCCACAGTTCCAGCTGGAGGTCAACA | |
| | GGAGCAGGAACAATAGCCGTTCAGGGTCCCAGTCCAGATCT | |
| | GTGTCCAGAAACAGGTCCCAGAGCAGGGGACGGCAGCAGA | |
| | GTAACAACCAGAATAATGTGGAAGACACCATAGTAGCAGTGC | |
| | TCCAGAAACTGGGGGTCACAGAAAAACAGAGGAGCAGGTC | |
| | CAAGTCTAGGGACCGTGGGGACTCTAAGCCAAGGGACACCA | |
| | CACCCAACAACGCCAACAAGCACACATGGAAAAAAACAGC | |
| | AGGGAAGGGTGATGTCACCAACTTTTACGGGGCCAGGTCAG | |
| | CCTCTGCAAACTTCGGGGATAGTGACCTGGTGGCCAACGGC | |
| | AATGCTGCTAAATCCTACCCTCAGATTGCTGAGTGCGTACCCT | |
| | CTGTATCCTCTATGCTCTTTGGCTCACAATGGTCTGCTGAGGA | |
| | TGATGGTGACCAGGTCAAGGTCACCTTGACCCATACCTACTA | |
| | TCTGCCCAAGGATGATGCAAAAACCAGCCAGTTCCTAGAGC | |
| | AGATAGATGCCTACAAGAGGCCCAGCCAGGTGGCCAAGGAT | |
| | CAGAGGCAGAGAAAGAGCAGATCCAAGAGCGCAGAAAAGA | |
| | AACCAGAGGAGTTATCTGTGACCCTGGTGGAGGCCTACACA | |
| | GATGTCTTTGATGATACACAGGTGGAAATGATAGATGAGGTG | |
| | ACTAAC | |
| 157 | ATGAGATTTGTGATGTCCCCCACTGTCCTGCTGCTGCTCCTGG | MHCIsp- |
| | GAGCCCTGGCAGCCCCCCAGACCTGGGCAGGCAGCGCCACA | N(N2)- |
| | CAGGGCCAGAGGGTGAACTGGGGCGACGAGCCATCCAAGA | MITD |
| | GGAGGGGAAGGAGCAACAGCAGAGGAAGGAAGAACAACA | |
| | CCATCCCCCTGTCCTTCTTCAACCCAATTCAGCTAGAGCCAG | |
| | GCAGCAAGTTCTGGTCAGTGTGCCCCAGAGACTTCGTGCCC | |
| | AAGGGCATCGGAAACAAGGACCAGCAGATCGGCTACTGGAA | |
| | CAGACAGGAGAGATACAGAATTGTGAAAGGCCAGAGAAAG | |
| | GAGCTGCCAGAGAGGTGGTTCTTCTACTTCCTGGGCACCGGC | |
| | CCACAGGCAGACGCCAAGTTCAAGGACAAGATCGATGGAGT | |
| | GTTCTGGGTGGCCAAGGACGGCGCCATGAACAAGCCCACCA | |
| | CACTGGGCACAAGAGGAACAAACAATGAGAGCAAGCCACT | |
| | GAAGTTTGATGGCAAGATCCCACCCCAGTTCCAGCTGGAGGT | |
| | GAACAGGAGCAGAAACAACAGCAGAAGCGGCAGCCAGAGC | |
| | AGAAGTGTGAGCAGAAACAGAAGCCAGAGCAGAGGAAGAC | |
| | AGCAGAGCAACAACCAGAACAACGTGGAGGACACCATCGT | |
| | GGCCGTGCTGCAGAAGCTGGGGGTCACAGAAAAGCAGAGG | |
| | AGCAGAAGCAAGAGCAGGGACAGAGGAGACAGCAAGCCAA | |
| | GAGACACCACCCCCAACAACGCCAACAAGCACACCTGGAA | |
| | GAAGACAGCCGGCAAGGGAGATGTGACCAACTTCTACGGCG | |
| | CCAGAAGCGCCAGCGCCAACTTCGGAGACTCAGACCTGGTG | |
| | GCCAATGGAAACGCAGCCAAGAGCTACCCCCAGATCGCAGA | |
| | GTGTGTGCCCTCTGTCTCCAGCATGCTGTTTGGCAGCCAGTG | |
| | GAGCGCCGAGGACGACGGTGACCAGGTGAAGGTGACCCTG | |
| | ACACACACATACTACCTGCCCAAAGATGACGCCAAGACCAG | |
| | CCAGTTCCTGGAGCAGATTGATGCCTACAAGAGGCCCAGCCA | |
| | GGTGGCCAAGGACCAGAGACAGAGGAAGAGCAGGTCCAAG | |
| | AGCGCCGAGAAGAAGCCAGAAGAATTGAGTGTCACCCTGGT | |
| | GGAGGCCTACACAGACGTGTTTGATGACACCCAGGTGGAGA | |
| | TGATTGATGAGGTGACCAACTTCCTGGGCATCATCGCAGGAG | |
| | TGGTGGTGCTGGTGGTGACAGTGGTGGTGGGAGCAGTGATC | |
| | TGGAGAAAGAAATGCAGCGGGAGAAAGGGCCCCAGCTACA | |
| | GCCACGCCGCCAGGGACGACAGCACCCAGGGCAGCGACAG | |
| | CAGCCTCATGGCCCCCAAGGTG | |
| 158 | ATGAGATTTGTGATGAGCCCCACTGTGCTGCTGCTGCTGCTG | MHCIsp- |
| | GGAGCCCTGGCAGCCCCCCAGACCTGGGCTGGCTCCGCCAC | N(N3)- |
| | CCAGGGCCAGAGAGTGAACTGGGGCGACGAGCCCTCAAAA | MITD |
| | AGGAGGGGCAGATCCAACAGCAGAGGCAGGAAGAACAACA | |
| | CCATCCCCCTGAGCTTCTTCAACCCCATCCAGCTGGAGCCAG | |
| | GCTCCAAGTTCTGGTCAGTGTGCCCAAGGGACTTCGTGCCCA | |
| | AGGGCATCGGCAACAAGGACCAGCAGATCGGCTACTGGAAC | |
| | AGGCAGGAGAGATACAGAATCGTGAAGGGCCAGAGGAAGG | |
| | AACTGCCAGAAAGGTGGTTCTTCTACTTCCTGGGCACCGGCC | |
| | CCCAGGCTGACGCCAAGTTCAAAGACAAGATCGACGGGGTG | |
| | TTCTGGGTGGCCAAGGACGGCGCCATGAACAAGCCAACAAC | |
| | ACTGGGCACCAGAGGAACCAACAACGAGAGCAAGCCACTG | |
| | AAGTTTGACGGCAAGATCCCCCCCCAGTTCCAGCTGGAAGT | |
| | CAACAGGAGCAGGAACAACAGCAGGTCCGGCTCACAAAGC | |
| | AGGAGCGTGTCCAGAAACAGATCCCAGTCAAGAGGAAGAC | |
| | AGCAGTCCAACAACCAGAACAACGTGGAGGACACCATAGTG | |
| | GCCGTGCTGCAGAAGCTGGGAGTCACAGAGAAGCAGAGGA | |
| | GCAGATCCAAGTCCAGGGACAGGGGAGACAGCAAGCCCAG | |
| | GGACACCACACCCAACAACGCCAACAAGCACACCTGGAAG | |
| | AAGACAGCCGGCAAGGGAGATGTGACCAACTTCTACGGCGC | |
| | CAGAAGCGCCTCAGCCAACTTCGGAGACTCAGACCTGGTGG | |
| | CCAACGGAAACGCCGCCAAGAGCTACCCCCAGATCGCCGAA | |
| | TGTGTCCCCTCAGTGTCCTCCATGCTCTTCGGCTCACAGTGG | |
| | TCAGCAGAGGACGACGGCGACCAGGTGAAGGTGACCCTGA | |
| | CCCACACCTACTACCTGCCCAAGGACGACGCCAAGACAAGC | |
| | CAGTTCCTGGAGCAGATCGACGCCTACAAGAGGCCATCCCA | |
| | GGTGGCCAAGGACCAGAGGCAGAGGAAGAGCAGAAGCAAG | |
| | TCAGCCGAGAAGAAACCAGAGGAGCTGTCAGTCACCCTGGT | |
| | GGAGGCCTACACCGACGTGTTCGACGACACCCAGGTGGAGA | |
| | TGATCGACGAGGTGACCAACTTCCTGGGCATCATCGCCGGCG | |
| | TGGTGGTCCTGGTGGTGACCGTGGTGGTCGGAGCAGTGATCT | |
| | GGAGGAAGAAGTGCTCAGGCAGGAAGGGCCCATCCTACAGC | |
| | CACGCCGCCAGAGATGACAGCACCCAAGGCTCAGACAGCTC | |
| | CCTGATGGCCCCCAAGGTG | |
| 159 | ATGCGATTCGTCATGTCACCAACCGTTTTACTATTATTACTAGG | MHCIsp- |
| | AGCATTAGCAGCACCGCAAACATGGGCAGGAAGTGCAACAC | N(N6)- |
| | AAGGACAGAGAGTAAATTGGGGGGATGAGCCCAGCAAGAG | MITD |
| | GCGAGGCAGAAGCAACTCAAGAGGGAGAAAAAACAATACC | |
| | ATCCCACTGTCATTCTTCAACCCCATTCAACTGGAGCCAGGC | |
| | TCTAAATTCTGGAGTGTATGCCCCAGGGACTTTGTGCCCAAG | |
| | GGCATAGGGAACAAGGACCAGCAAATAGGATACTGGAACCG | |
| | GCAGGAGAGATACAGAATTGTCAAGGGTCAGAGAAAGGAGC | |
| | TGCCAGAGAGATGGTTCTTCTACTTCCTAGGAACAGGCCCAC | |
| | AGGCAGACGCTAAGTTCAAGGATAAGATCGATGGTGTCTTCT | |
| | GGGTCGCCAAGGATGGTGCAATGAATAAACCAACCACCCTG | |
| | GGGACCAGGGGGACAAACAATGAGTCCAAGCCCCTCAAGTT | |
| | TGATGGCAAAATCCCCCCACAGTTCCAGCTGGAGGTCAACA | |
| | GGAGCAGGAACAATAGCCGTTCAGGGTCCCAGTCCAGATCT | |
| | GTGTCCAGAAACAGGTCCCAGAGCAGGGGACGGCAGCAGA | |
| | GTAACAACCAGAATAATGTGGAAGACACCATAGTAGCAGTGC | |
| | TCCAGAAACTGGGGGTCACAGAAAAACAGAGGAGCAGGTC | |
| | CAAGTCTAGGGACCGTGGGGACTCTAAGCCAAGGGACACCA | |
| | CACCCAACAACGCCAACAAGCACACATGGAAAAAAACAGC | |
| | AGGGAAGGGTGATGTCACCAACTTTTACGGGGCCAGGTCAG | |
| | CCTCTGCAAACTTCGGGGATAGTGACCTGGTGGCCAACGGC | |
| | AATGCTGCTAAATCCTACCCTCAGATTGCTGAGTGCGTACCCT | |
| | CTGTATCCTCTATGCTCTTTGGCTCACAATGGTCTGCTGAGGA | |
| | TGATGGTGACCAGGTCAAGGTCACCTTGACCCATACCTACTA | |
| | TCTGCCCAAGGATGATGCAAAAACCAGCCAGTTCCTAGAGC | |
| | AGATAGATGCCTACAAGAGGCCCAGCCAGGTGGCCAAGGAT | |
| | CAGAGGCAGAGAAAGAGCAGATCCAAGAGCGCAGAAAAGA | |
| | AACCAGAGGAGTTATCTGTGACCCTGGTGGAGGCCTACACA | |
| | GATGTCTTTGATGATACACAGGTGGAAATGATAGATGAGGTG | |
| | ACTAACTTCCTTGGAATCATAGCTGGGGTCGTTGTCCTCGTAG | |
| | TGACTGTAGTGGTAGGCGCAGTTATCTGGAGGAAGAAATGCT | |
| | CGGGGAGGAAAGGGCCCTCTTACAGCCATGCTGCCAGGGAT | |
| | GACTCCACACAGGGGTCAGATAGCAGCCTCATGGCCCCAAA | |
| | GGTC | |
| 160 | ATGACCACCAACAATGAATGCATCCAGGTGAACGTGACCCA | S(S2) |
| | GCTGGCAGGCAATGAAAATTTGATCAGAGACTTCCTGTTCAG | |
| | CAACTTCAAGGAGGAGGGCAGTGTAGTGGTGGGAGGCTACT | |
| | ACCCAACAGAGGTGTGGTACAACTGCAGCAGAACAGCCAGA | |
| | ACCACAGCCTTCCAGTACTTCAACAACATCCACGCCTTCTAC | |
| | TTTGTGATGGAGGCCATGGAAAACAGCACAGGAAATGCCAG | |
| | AGGAAAACCCCTGCTCTTCCACGTGCACGGAGAGCCCGTGT | |
| | CAGTCATCATCAGCGCCTACAGAGATGACGTCCAGCAGCGGC | |
| | CCCTGCTGAAGCATGGACTGGTCTGCATCACCAAGAACAGA | |
| | CACATCAACTACGAGCAGTTCACCAGCAACCAGTGGAACAG | |
| | CACCTGCACAGGAGCAGACAGAAAAATCCCCTTCAGCGTCA | |
| | TCCCCACAGACAACGGCACCAAAATCTATGGCCTGGAGTGG | |
| | AATGATGACTTTGTGACAGCCTATATCAGCGGCAGGAGCTAC | |
| | CACCTCAACATCAACACCAACTGGTTCAACAACGTCACCCTG | |
| | CTCTACTCCAGATCCAGCACAGCCACCTGGGAGTACAGCGCC | |
| | GCCTATGCCTACCAGGGAGTCTCCAACTTCACCTACTACAAA | |
| | CTGAACAACACCAACGGCCTGAAAACCTACGAGCTGTGTGA | |
| | GGACTACGAGCACTGCACAGGCTATGCCACAAATGTGTTTGC | |
| | CCCAACCAGCGGAGGCTACATCCCAGACGGCTTCTCCTTCAA | |
| | CAACTGGTTCCTCCTCACCAACTCCTCCACATTTGTGAGCGG | |
| | CAGATTTGTGACCAACCAGCCCCTGCTGATCAACTGCCTGTG | |
| | GCCCGTGCCCAGCTTTGGAGTGGCAGCCCAGGAGTTCTGCTT | |
| | CGAGGGAGCCCAGTTCAGCCAGTGCAACGGAGTCAGCCTGA | |
| | ACAACACAGTGGACGTGATCAGATTCAACCTGAACTTCACA | |
| | GCAGACGTGCAGAGTGGAATGGGAGCCACCGTCTTCAGCCT | |
| | GAACACCACAGGAGGAGTGATCCTGGAGATCAGCTGCTACA | |
| | GCGACACAGTGAGCGAGAGCAGCAGCTACAGCTACGGAGA | |
| | GATCCCATTTGGCATCACAGATGGCCCCAGGTACTGCTACGT | |
| | CCTGTACAATGGAACAGCCCTGAAATACCTGGGCACCCTCCC | |
| | ACCCAGCGTGAAGGAGATCGCCATCAGCAAGTGGGGCCACT | |
| | TCTACATCAATGGCTACAACTTCTTCAGCACCTTCCCCATCGG | |
| | CTGCATCTCCTTCAACCTGACCACAGGAGTGAGCGGGGCCTT | |
| | CTGGACAATCGCCTACACATCCTACACAGAAGCCCTGGTGCA | |
| | GGTGGAGAACACAGCCATCAAAAACGTCACCTACTGCAACA | |
| | GCCACATCAACAACATCAAGTGCAGCCAGCTGACAGCCAAC | |
| | CTGAACAACGGCTTCTACCCAGTGGCCAGCTCAGAGGTGGG | |
| | CTTCGTGAACAAGAGCGTGGTGCTCCTGCCCAGCTTCTTCAC | |
| | CTACACAGCAGTGAACATCACAATTGACCTGGGCATGAAGCT | |
| | GAGCGGCTACGGCCAGCCAATTGCCAGCACCCTCTCCAACAT | |
| | CACCCTCCCCATGCAGGACAATAACACAGATGTGTACTGCAT | |
| | CAGATCCAACCAGTTCTCTGTCTACGTGCACAGCACCTGCAA | |
| | AAGCAGCCTGTGGGACAACATCTTCAACCAGGACTGCACAG | |
| | ATGTCCTGGAGGCCACAGCCGTGATCAAAACAGGCACCTGC | |
| | CCCTTCAGCTTTGACAAACTCAACAACTACCTTACATTCAAC | |
| | AAATTCTGCCTCTCCCTCAGCCCAGTGGGAGCCAACTGCAAG | |
| | TTTGATGTGGCCGCCAGGACCAGGACAAATGAACAAGTGGT | |
| | CAGAAGCCTCTACGTCATCTACGAGGAGGGAGACAACATCG | |
| | TGGGGGTCCCCAGCGACAACAGCGGCCTGCACGACCTGAGT | |
| | GTGCTCCACCTGGACAGCTGCACAGACTACAACATCTACGGC | |
| | AGGACTGGGGTGGGCATCATCAGAAGAACCAACAGCACACT | |
| | GCTGAGTGGCCTGTACTACACCAGCCTGAGTGGAGACTTGCT | |
| | GGGCTTCAAGAATGTGTCAGATGGGGTGATCTACAGTGTGAC | |
| | CCCCTGTGACGTGTCTGCCCAGGCTGCAGTCATCGACGGAGC | |
| | CATCGTGGGAGCCATGACCAGCATTAACAGCGAGCTGCTGGG | |
| | CCTGACCCACTGGACCACCACCCCCAACTTCTACTACTACTC | |
| | CATCTACAACTACACATCAGAAAGAACAAGAGACACAGCCA | |
| | TCGACAGCAATGACGTGGACTGTGAGCCAGTCATCACCTACA | |
| | GCAACATCGGAGTGTGCAAGAACGGAGCCCTGGTGTTCATC | |
| | AACGTGACCCACAGCGACGGAGATGTCCAGCCCATCAGCAC | |
| | AGGAAATGTGACCATCCCAACCAACTTCACCATCAGCGTCCA | |
| | GGTGGAATACATGCAGGTGTACACCACCCCAGTGTCCATCGA | |
| | CTGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCCTGGTATGTGTGGCTGCTGATTGG | |
| | ACTGGTGGTGGTGTTCTGCATCCCACTGCTGCTGTTCTGCTG | |
| | CTTCAGCACCGGCTGCTGTGGATGCATCGGCTGCTTGGGCAG | |
| | CTGCTGCCACAGCATCTGCAGCAGGAGGCAGTTTGAGAACT | |
| | ACGAACCAATTGAAAAAGTGCACGTCCAC | |
| 161 | ATGACCACCAACAACGAGTGCATCCAGGTGAACGTGACCCA | S(S3) |
| | GCTGGCAGGCAACGAGAACCTCATCAGAGACTTCCTCTTCTC | |
| | CAACTTCAAGGAGGAGGGCTCAGTGGTGGTCGGCGGCTACT | |
| | ACCCAACAGAGGTGTGGTACAACTGCTCAAGGACCGCCAGA | |
| | ACCACAGCCTTCCAGTACTTCAACAACATCCACGCCTTCTAC | |
| | TTCGTGATGGAGGCCATGGAGAACTCCACCGGGAACGCCAG | |
| | GGGCAAGCCACTACTCTTCCACGTGCACGGAGAGCCAGTGA | |
| | GCGTGATCATCTCAGCCTACAGGGACGACGTGCAGCAGCGC | |
| | CCCCTGCTGAAGCATGGACTGGTGTGCATCACCAAGAACAG | |
| | GCACATCAACTACGAGCAGTTCACCAGCAACCAGTGGAACA | |
| | GCACCTGCACCGGCGCAGACAGGAAGATCCCCTTCTCAGTG | |
| | ATCCCAACAGACAACGGAACCAAAATCTACGGCCTGGAGTG | |
| | GAACGACGACTTCGTGACCGCCTACATCAGCGGCAGGTCCTA | |
| | CCATCTCAACATCAACACCAACTGGTTCAACAACGTCACCCT | |
| | CCTCTACAGCAGGTCATCCACAGCCACCTGGGAGTACTCAGC | |
| | TGCCTATGCATACCAGGGAGTCTCCAACTTCACATACTACAA | |
| | ACTCAACAACACCAACGGCCTCAAGACCTACGAGCTGTGTG | |
| | AGGACTACGAGCACTGCACCGGCTACGCAACAAACGTCTTC | |
| | GCCCCAACCTCCGGAGGCTACATCCCAGACGGCTTCTCCTTC | |
| | AACAACTGGTTCCTCCTCACAAACAGCTCCACCTTCGTGTCA | |
| | GGAAGGTTCGTGACCAACCAGCCCCTGCTCATCAACTGCCTC | |
| | TGGCCCGTCCCCTCCTTCGGAGTGGCCGCCCAGGAGTTCTGC | |
| | TTCGAGGGAGCCCAGTTCTCCCAGTGCAACGGAGTCTCCCTC | |
| | AACAACACCGTGGACGTCATCAGATTCAACCTCAACTTCACA | |
| | GCAGACGTCCAGAGCGGCATGGGAGCCACCGTGTTCAGCCT | |
| | GAACACCACAGGAGGAGTGATCCTGGAGATCTCCTGCTACTC | |
| | AGACACAGTGTCAGAGTCCTCCTCCTACAGCTACGGAGAGAT | |
| | CCCATTCGGCATCACAGACGGCCCCAGATACTGCTACGTGCT | |
| | GTACAACGGCACAGCCCTGAAGTACCTGGGCACCCTCCCCC | |
| | CATCAGTGAAGGAGATCGCCATCAGCAAGTGGGGCCACTTCT | |
| | ACATCAACGGCTACAACTTCTTCTCCACCTTCCCCATCGGCT | |
| | GCATCAGCTTCAACCTGACCACCGGAGTGTCCGGAGCCTTCT | |
| | GGACCATCGCCTACACATCATACACCGAGGCCCTGGTGCAGG | |
| | TGGAGAACACAGCCATAAAGAACGTGACCTACTGCAACAGC | |
| | CACATCAACAACATCAAGTGCTCCCAGCTGACAGCCAACCT | |
| | GAACAACGGCTTCTACCCAGTGGCCTCCAGCGAGGTGGGCT | |
| | TCGTGAACAAGAGCGTGGTCCTACTCCCCTCCTTCTTCACCT | |
| | ACACAGCAGTCAACATCACAATTGACCTGGGCATGAAGCTGT | |
| | CCGGCTACGGCCAGCCAATCGCCAGCACCCTGTCCAACATCA | |
| | CCCTGCCAATGCAGGACAACAACACCGACGTCTACTGCATCA | |
| | GAAGCAACCAGTTCTCCGTGTACGTCCACTCCACCTGCAAGT | |
| | CCTCCCTCTGGGACAACATCTTCAACCAGGACTGCACAGAC | |
| | GTGCTGGAGGCCACAGCTGTGATCAAGACAGGAACCTGCCC | |
| | TTTCTCATTCGACAAGCTCAACAACTACCTGACCTTCAACAA | |
| | GTTCTGCCTGAGCCTGTCCCCAGTGGGAGCCAACTGCAAGTT | |
| | CGACGTGGCCGCCAGAACCAGGACCAACGAGCAGGTGGTC | |
| | AGAAGCCTGTACGTCATCTACGAGGAGGGAGACAACATCGT | |
| | GGGAGTGCCCAGCGACAACTCAGGCCTGCACGACCTGAGCG | |
| | TGCTGCACCTGGACTCCTGCACAGACTACAACATCTACGGCA | |
| | GGACAGGAGTGGGCATCATCAGGAGGACCAACAGCACACTG | |
| | CTGTCCGGCCTCTACTACACCTCCCTGTCCGGAGACTTGCTG | |
| | GGATTCAAGAACGTGTCAGACGGAGTCATCTACAGCGTCACC | |
| | CCATGTGACGTGAGCGCCCAGGCAGCAGTGATAGACGGAGC | |
| | CATCGTGGGAGCCATGACCTCAATCAACTCAGAACTGCTGGG | |
| | CCTCACCCACTGGACAACAACACCCAACTTCTACTACTACTC | |
| | CATCTACAACTACACATCAGAAAGAACAAGGGACACAGCAA | |
| | TCGACTCCAACGACGTGGACTGTGAGCCAGTCATCACCTACT | |
| | CCAACATCGGCGTGTGCAAGAACGGAGCCCTGGTGTTCATC | |
| | AACGTCACCCACTCAGACGGCGACGTCCAGCCAATCTCCAC | |
| | AGGAAACGTCACCATCCCCACCAACTTCACCATCAGCGTGCA | |
| | GGTGGAGTACATGCAGGTCTACACCACCCCAGTCTCCATCGA | |
| | CTGTGCCAGGTACGTGTGCAACGGCAACCCAAGATGCAACA | |
| | AACTGCTGACCCAGTACGTGAGCGCCTGCCAGACCATCGAG | |
| | CAGGCCCTGGCCATGGGCGCCAGGCTGGAGAACATGGAGGT | |
| | GGACAGCATGCTCTTTGTGAGCGAGAACGCCCTGAAGCTTG | |
| | CCAGCGTGGAGGCCTTCAACAGCACCGAAAACCTGGACTCC | |
| | ATCTACAAAGAGTGGCCCTCCATAGGAGGCTCCTGGCTGGGA | |
| | GGCCTGAAGGACATCCTCCCATCCCACAACAGCAAAAGAAA | |
| | GTACGGCAGCGCCATCGAAGACCTGCTGTTCGACAAGGTGG | |
| | TCACCTCAGGACTGGGCACAGTGGACGAGGACTACAAGAGG | |
| | TGCACCGGAGGCTACGACATCGCAGACCTGGTCTGTGCCCA | |
| | GTACTACAACGGCATCATGGTGCTCCCAGGCGTGGCCAACGC | |
| | CGACAAGATGACCATGTACACAGCAAGCCTGGCTGGAGGAA | |
| | TCACACTGGGAGCCCTGGGAGGAGGGGCCGTGGCCATTCCA | |
| | TTCGCCGTGGCCGTGCAGGCCAGACTGAACTACGTGGCCCT | |
| | GCAGACAGACGTGCTAAACAAGAACCAGCAGATCCTGGCCA | |
| | ACGCCTTCAACCAGGCCATCGGCAACATCACCCAGGCCTTCG | |
| | GCAAGGTGAACGACGCAATCCACCAGACATCACAGGGCCTG | |
| | GCAACAGTGGCCAAGGCCCTGGCCAAGGTCCAGGACGTGGT | |
| | GAACACCCAGGGCCAGGCCCTCTCACACCTGACAGTCCAGC | |
| | TGCAGAACAACTTCCAGGCAATCTCCTCCTCCATCTCAGACA | |
| | TCTACAACAGACTGGACCCCCCCTCAGCCGACGCCCAGGTG | |
| | GACAGACTCATCACAGGCAGGCTGACCGCCCTCAACGCCTT | |
| | CGTGTCCCAGACCCTCACCAGGCAGGCCGAGGTGAGGGCCA | |
| | GCAGGCAGCTCGCCAAGGACAAGGTGAACGAGTGCGTCAG | |
| | AAGCCAGAGCCAGAGGTTCGGCTTCTGTGGCAACGGCACCC | |
| | ACCTGTTCTCCCTGGCCAACGCAGCCCCCAACGGCATGATCT | |
| | TCTTCCACACAGTCCTCCTCCCAACAGCATATGAGACAGTCA | |
| | CCGCCTGGTCAGGAATCTGTGCCTCAGACGGGGACAGAACC | |
| | TTCGGCCTGGTGGTCAAGGACGTGCAGCTGACACTCTTCAG | |
| | AAACCTGGACGACAAATTCTACCTGACCCCCAGGACCATGTA | |
| | CCAGCCAAGGGTGGCCACCTCCTCAGACTTCGTGCAGATCG | |
| | AGGGCTGTGACGTGCTCTTCGTGAACGCCACCGTCATCGACC | |
| | TCCCATCCATCATCCCAGACTACATCGACATCAACCAGACAG | |
| | TGCAGGACATCCTGGAGAACTACCGCCCCAACTGGACCGTG | |
| | CCAGAGTTCACCCTAGACATATTCAACGCCACCTACCTGAAC | |
| | CTGACAGGAGAAATTGACGACCTGGAGTTCAGATCAGAAAA | |
| | GCTACACAACACCACCGTGGAGTTAGCCATCCTCATAGACAA | |
| | CATTAACAACACCCTCGTCAACCTGGAGTGGCTCAACAGGAT | |
| | TGAAACCTACGTGAAGTGGCCCTGGTACGTCTGGCTCCTCAT | |
| | CGGCCTGGTGGTGGTCTTCTGCATCCCACTGCTGCTGTTCTG | |
| | CTGCTTCTCCACCGGCTGCTGTGGATGCATCGGCTGCCTGGG | |
| | CTCATGCTGCCACTCAATCTGCTCAAGGAGGCAGTTTGAAAA | |
| | CTACGAGCCAATAGAAAAAGTCCACGTCCAC | |
| 162 | ATGACCACAAATAACGAGTGCATTCAGGTCAACGTCACCCAG | S(S6) |
| | CTGGCCGGTAACGAGAACCTAATTAGAGACTTCCTATTCTCG | |
| | AACTTTAAAGAGGAAGGCTCTGTGGTGGTCGGAGGTTACTA | |
| | CCCCACAGAAGTGTGGTACAATTGCTCACGTACAGCCAGGA | |
| | CCACTGCCTTCCAGTACTTCAACAACATTCATGCCTTCTACTT | |
| | TGTCATGGAAGCCATGGAGAACTCCACTGGGAATGCCAGAG | |
| | GAAAGCCTCTCCTCTTCCATGTCCATGGAGAGCCTGTCTCTG | |
| | TGATTATCTCAGCATATAGGGATGATGTGCAGCAGCGGCCGCT | |
| | GCTTAAGCATGGCCTAGTGTGCATTACTAAGAACCGACATATC | |
| | AATTATGAGCAGTTCACCTCCAACCAGTGGAACTCCACATGC | |
| | ACTGGTGCTGATAGGAAGATCCCGTTCAGCGTTATCCCCACC | |
| | GATAATGGCACAAAGATTTATGGCCTAGAATGGAACGATGAT | |
| | TTTGTTACTGCCTACATATCAGGAAGAAGTTACCACTTAAACA | |
| | TTAACACCAATTGGTTCAATAATGTTACACTTCTGTACTCTCG | |
| | CAGCAGTACGGCCACTTGGGAGTATTCGGCTGCATATGCCTA | |
| | CCAAGGTGTAAGCAACTTCACCTACTACAAGCTGAACAATAC | |
| | GAACGGTCTGAAGACTTATGAGCTGTGCGAAGACTACGAGC | |
| | ACTGTACGGGCTATGCGACAAATGTCTTCGCCCCGACGAGCG | |
| | GCGGGTACATACCGGATGGCTTCTCCTTCAACAACTGGTTCC | |
| | TCCTTACCAATAGCTCCACTTTCGTATCAGGAAGATTTGTTAC | |
| | GAACCAACCCCTTCTCATTAACTGTCTGTGGCCAGTGCCCTC | |
| | CTTCGGAGTAGCTGCTCAAGAGTTCTGTTTCGAGGGTGCACA | |
| | GTTCAGCCAGTGTAATGGAGTGTCGCTGAACAACACTGTGG | |
| | ACGTGATCAGGTTTAATTTGAACTTCACAGCTGATGTTCAGTC | |
| | CGGCATGGGCGCGACTGTGTTCAGCCTAAACACCACGGGTG | |
| | GCGTCATCTTGGAGATTAGTTGTTACTCTGACACTGTGTCAG | |
| | AGAGCAGCAGTTACTCCTACGGAGAAATTCCTTTCGGCATCA | |
| | CAGACGGTCCCCGGTACTGCTATGTGCTGTACAACGGAACTG | |
| | CTTTGAAGTACCTGGGGACATTGCCACCTTCTGTGAAGGAAA | |
| | TAGCCATCTCTAAGTGGGGTCACTTTTACATTAACGGCTATAA | |
| | TTTCTTTTCCACTTTCCCAATTGGATGCATTAGCTTCAACCTG | |
| | ACAACAGGTGTGTCTGGAGCCTTCTGGACCATCGCCTATACC | |
| | TCTTACACAGAGGCTCTAGTACAGGTGGAGAACACAGCTATA | |
| | AAGAACGTGACGTACTGTAACAGTCACATAAACAATATCAAG | |
| | TGTTCTCAGTTGACTGCGAACTTAAACAATGGGTTTTATCCA | |
| | GTGGCGAGCTCGGAGGTGGGGTTTGTAAACAAATCTGTGGT | |
| | GCTGTTGCCCTCCTTCTTCACGTACACTGCAGTGAACATCAC | |
| | CATTGATTTGGGGATGAAACTGTCCGGCTACGGGCAGCCTAT | |
| | AGCATCTACACTGAGCAATATCACACTGCCCATGCAGGATAA | |
| | CAATACAGATGTGTACTGTATCCGCTCAAACCAGTTCTCTGTA | |
| | TACGTGCACAGTACATGCAAGAGCTCGCTATGGGACAACATT | |
| | TTCAACCAGGATTGTACTGATGTGCTTGAAGCAACTGCAGTG | |
| | ATCAAAACAGGCACATGCCCGTTCAGCTTTGATAAGCTCAAC | |
| | AACTACCTAACGTTCAACAAGTTCTGCTTGAGCCTGTCTCCA | |
| | GTAGGCGCCAATTGCAAGTTTGACGTTGCAGCGCGAACACG | |
| | GACAAACGAACAGGTAGTGCGGTCGCTCTATGTTATCTACGA | |
| | GGAGGGGGACAACATAGTCGGGGTTCCATCCGACAACTCAG | |
| | GTTTGCACGACCTGAGTGTGCTCCATTTGGACTCATGCACGG | |
| | ATTATAACATCTACGGGCGCACAGGTGTGGGGATAATACGAA | |
| | GAACAAACTCTACGCTATTGAGCGGGCTCTACTACACCTCAT | |
| | TGAGTGGGGACCTGCTAGGGTTCAAGAACGTATCTGACGGT | |
| | GTGATCTATAGCGTCACACCATGTGACGTATCAGCCCAAGCT | |
| | GCTGTGATTGACGGGGCGATTGTGGGGGCTATGACTTCAATT | |
| | AACAGCGAGCTCCTAGGCCTGACCCACTGGACTACCACCCC | |
| | AAACTTCTACTACTACAGCATTTATAACTATACCAGTGAGCGC | |
| | ACCAGGGACACTGCCATTGACAGCAATGACGTCGACTGCGA | |
| | GCCTGTTATTACCTACAGCAACATCGGTGTTTGTAAGAATGGA | |
| | GCTCTAGTCTTCATAAACGTAACGCACTCTGATGGCGATGTTC | |
| | AACCAATTTCCACTGGGAACGTAACCATACCCACCAACTTTA | |
| | CTATTTCCGTCCAGGTGGAGTACATGCAAGTATATACCACGCC | |
| | AGTGTCCATCGACTGCGCTCGGTATGTGTGCAACGGTAACCC | |
| | ACGCTGCAATAAGCTGCTAACGCAGTACGTCAGCGCCTGCCA | |
| | GACAATAGAGCAGGCATTGGCAATGGGTGCAAGGCTTGAAA | |
| | ACATGGAGGTGGACTCCATGTTGTTCGTGTCTGAAAACGCTC | |
| | TTAAACTAGCATCCGTGGAGGCATTCAACAGTACTGAGAACT | |
| | TGGACTCTATCTATAAGGAGTGGCCCTCCATTGGGGGCAGCT | |
| | GGCTTGGAGGTCTAAAAGACATCCTGCCCAGCCACAACTCC | |
| | AAGAGGAAGTACGGGTCCGCTATAGAGGACCTCCTCTTTGAC | |
| | AAGGTTGTTACTTCTGGTCTTGGCACAGTGGACGAAGACTAC | |
| | AAGAGGTGCACAGGAGGCTATGATATAGCTGACCTGGTGTGT | |
| | GCTCAATACTACAACGGTATAATGGTTCTCCCAGGTGTGGCC | |
| | AACGCTGACAAGATGACAATGTACACAGCCTCTTTAGCTGGA | |
| | GGCATTACCCTGGGAGCCCTTGGGGGTGGCGCAGTGGCAATT | |
| | CCATTTGCCGTTGCGGTGCAGGCCCGACTAAACTATGTCGCA | |
| | CTTCAAACAGATGTGCTCAACAAGAACCAACAAATACTGGC | |
| | CAACGCTTTCAACCAGGCCATTGGTAACATTACGCAGGCATT | |
| | TGGCAAGGTGAATGACGCCATCCACCAGACCAGCCAGGGAC | |
| | TTGCCACAGTGGCCAAGGCCTTGGCAAAGGTGCAGGATGTC | |
| | GTGAACACACAGGGTCAGGCCCTCTCTCATTTGACAGTGCA | |
| | GCTTCAGAATAACTTCCAAGCAATCAGTTCAAGCATCAGCGA | |
| | CATCTACAACCGGCTGGACCCCCCATCTGCAGATGCGCAGGT | |
| | GGACAGGCTAATCACTGGACGCTTGACGGCACTAAATGCCTT | |
| | TGTCAGCCAAACTCTGACCCGGCAAGCAGAGGTGCGGGCCA | |
| | GTAGACAACTGGCCAAAGACAAGGTCAACGAGTGCGTCAGG | |
| | TCCCAGTCCCAGCGTTTTGGATTCTGTGGGAACGGGACGCAC | |
| | CTGTTCTCATTAGCCAATGCTGCACCCAATGGCATGATCTTTT | |
| | TCCATACTGTTCTACTTCCTACTGCCTATGAAACCGTGACCGC | |
| | TTGGAGCGGCATCTGCGCATCTGATGGCGATAGGACCTTCGG | |
| | GCTGGTCGTTAAGGATGTCCAGCTAACGCTGTTCCGGAACTT | |
| | GGATGACAAGTTCTACCTGACCCCCAGGACCATGTACCAGCC | |
| | GAGAGTGGCAACGAGTTCTGACTTCGTGCAAATTGAGGGCT | |
| | GTGACGTCCTGTTTGTTAATGCAACAGTGATCGATCTGCCCA | |
| | GTATCATACCAGATTACATAGACATAAACCAGACAGTCCAGG | |
| | ACATACTGGAGAATTACAGGCCAAACTGGACCGTACCAGAG | |
| | TTCACGCTGGACATATTCAACGCTACGTACCTCAATTTGACTG | |
| | GGGAAATTGATGACTTGGAGTTCAGGTCGGAGAAGCTCCAC | |
| | AACACCACTGTGGAGCTGGCCATCCTGATTGACAACATCAAC | |
| | AACACTCTGGTGAACCTGGAGTGGCTAAATCGCATTGAAACC | |
| | TATGTCAAGTGGCCTTGGTACGTTTGGCTACTGATCGGACTC | |
| | GTGGTAGTCTTCTGCATACCACTCCTGCTATTTTGCTGCTTCA | |
| | GCACAGGGTGCTGTGGCTGCATTGGATGCCTAGGTTCCTGCT | |
| | GTCACAGTATCTGCAGCAGAAGACAATTCGAGAACTACGAG | |
| | CCCATAGAAAAGGTCCACGTACAT | |
| 163 | ATGACCACCAACAATGAATGCATCCAGGTGAACGTGACCCA | S_ec(S2) |
| | GCTGGCAGGCAATGAAAATTTGATCAGAGACTTCCTGTTCAG | |
| | CAACTTCAAGGAGGAGGGCAGTGTAGTGGTGGGAGGCTACT | |
| | ACCCAACAGAGGTGTGGTACAACTGCAGCAGAACAGCCAGA | |
| | ACCACAGCCTTCCAGTACTTCAACAACATCCACGCCTTCTAC | |
| | TTTGTGATGGAGGCCATGGAAAACAGCACAGGAAATGCCAG | |
| | AGGAAAACCCCTGCTCTTCCACGTGCACGGAGAGCCCGTGT | |
| | CAGTCATCATCAGCGCCTACAGAGATGACGTCCAGCAGCGGC | |
| | CCCTGCTGAAGCATGGACTGGTCTGCATCACCAAGAACAGA | |
| | CACATCAACTACGAGCAGTTCACCAGCAACCAGTGGAACAG | |
| | CACCTGCACAGGAGCAGACAGAAAAATCCCCTTCAGCGTCA | |
| | TCCCCACAGACAACGGCACCAAAATCTATGGCCTGGAGTGG | |
| | AATGATGACTTTGTGACAGCCTATATCAGCGGCAGGAGCTAC | |
| | CACCTCAACATCAACACCAACTGGTTCAACAACGTCACCCTG | |
| | CTCTACTCCAGATCCAGCACAGCCACCTGGGAGTACAGCGCC | |
| | GCCTATGCCTACCAGGGAGTCTCCAACTTCACCTACTACAAA | |
| | CTGAACAACACCAACGGCCTGAAAACCTACGAGCTGTGTGA | |
| | GGACTACGAGCACTGCACAGGCTATGCCACAAATGTGTTTGC | |
| | CCCAACCAGCGGAGGCTACATCCCAGACGGCTTCTCCTTCAA | |
| | CAACTGGTTCCTCCTCACCAACTCCTCCACATTTGTGAGCGG | |
| | CAGATTTGTGACCAACCAGCCCCTGCTGATCAACTGCCTGTG | |
| | GCCCGTGCCCAGCTTTGGAGTGGCAGCCCAGGAGTTCTGCTT | |
| | CGAGGGAGCCCAGTTCAGCCAGTGCAACGGAGTCAGCCTGA | |
| | ACAACACAGTGGACGTGATCAGATTCAACCTGAACTTCACA | |
| | GCAGACGTGCAGAGTGGAATGGGAGCCACCGTCTTCAGCCT | |
| | GAACACCACAGGAGGAGTGATCCTGGAGATCAGCTGCTACA | |
| | GCGACACAGTGAGCGAGAGCAGCAGCTACAGCTACGGAGA | |
| | GATCCCATTTGGCATCACAGATGGCCCCAGGTACTGCTACGT | |
| | CCTGTACAATGGAACAGCCCTGAAATACCTGGGCACCCTCCC | |
| | ACCCAGCGTGAAGGAGATCGCCATCAGCAAGTGGGGCCACT | |
| | TCTACATCAATGGCTACAACTTCTTCAGCACCTTCCCCATCGG | |
| | CTGCATCTCCTTCAACCTGACCACAGGAGTGAGCGGGGCCTT | |
| | CTGGACAATCGCCTACACATCCTACACAGAAGCCCTGGTGCA | |
| | GGTGGAGAACACAGCCATCAAAAACGTCACCTACTGCAACA | |
| | GCCACATCAACAACATCAAGTGCAGCCAGCTGACAGCCAAC | |
| | CTGAACAACGGCTTCTACCCAGTGGCCAGCTCAGAGGTGGG | |
| | CTTCGTGAACAAGAGCGTGGTGCTCCTGCCCAGCTTCTTCAC | |
| | CTACACAGCAGTGAACATCACAATTGACCTGGGCATGAAGCT | |
| | GAGCGGCTACGGCCAGCCAATTGCCAGCACCCTCTCCAACAT | |
| | CACCCTCCCCATGCAGGACAATAACACAGATGTGTACTGCAT | |
| | CAGATCCAACCAGTTCTCTGTCTACGTGCACAGCACCTGCAA | |
| | AAGCAGCCTGTGGGACAACATCTTCAACCAGGACTGCACAG | |
| | ATGTCCTGGAGGCCACAGCCGTGATCAAAACAGGCACCTGC | |
| | CCCTTCAGCTTTGACAAACTCAACAACTACCTTACATTCAAC | |
| | AAATTCTGCCTCTCCCTCAGCCCAGTGGGAGCCAACTGCAAG | |
| | TTTGATGTGGCCGCCAGGACCAGGACAAATGAACAAGTGGT | |
| | CAGAAGCCTCTACGTCATCTACGAGGAGGGAGACAACATCG | |
| | TGGGGGTCCCCAGCGACAACAGCGGCCTGCACGACCTGAGT | |
| | GTGCTCCACCTGGACAGCTGCACAGACTACAACATCTACGGC | |
| | AGGACTGGGGTGGGCATCATCAGAAGAACCAACAGCACACT | |
| | GCTGAGTGGCCTGTACTACACCAGCCTGAGTGGAGACTTGCT | |
| | GGGCTTCAAGAATGTGTCAGATGGGGTGATCTACAGTGTGAC | |
| | CCCCTGTGACGTGTCTGCCCAGGCTGCAGTCATCGACGGAGC | |
| | CATCGTGGGAGCCATGACCAGCATTAACAGCGAGCTGCTGGG | |
| | CCTGACCCACTGGACCACCACCCCCAACTTCTACTACTACTC | |
| | CATCTACAACTACACATCAGAAAGAACAAGAGACACAGCCA | |
| | TCGACAGCAATGACGTGGACTGTGAGCCAGTCATCACCTACA | |
| | GCAACATCGGAGTGTGCAAGAACGGAGCCCTGGTGTTCATC | |
| | AACGTGACCCACAGCGACGGAGATGTCCAGCCCATCAGCAC | |
| | AGGAAATGTGACCATCCCAACCAACTTCACCATCAGCGTCCA | |
| | GGTGGAATACATGCAGGTGTACACCACCCCAGTGTCCATCGA | |
| | CTGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCC | |
| 164 | ATGACCACCAACAACGAGTGCATCCAGGTGAACGTGACCCA | S_ec(S3) |
| | GCTGGCAGGCAACGAGAACCTCATCAGAGACTTCCTCTTCTC | |
| | CAACTTCAAGGAGGAGGGCTCAGTGGTGGTCGGCGGCTACT | |
| | ACCCAACAGAGGTGTGGTACAACTGCTCAAGGACCGCCAGA | |
| | ACCACAGCCTTCCAGTACTTCAACAACATCCACGCCTTCTAC | |
| | TTCGTGATGGAGGCCATGGAGAACTCCACCGGGAACGCCAG | |
| | GGGCAAGCCACTACTCTTCCACGTGCACGGAGAGCCAGTGA | |
| | GCGTGATCATCTCAGCCTACAGGGACGACGTGCAGCAGCGC | |
| | CCCCTGCTGAAGCATGGACTGGTGTGCATCACCAAGAACAG | |
| | GCACATCAACTACGAGCAGTTCACCAGCAACCAGTGGAACA | |
| | GCACCTGCACCGGCGCAGACAGGAAGATCCCCTTCTCAGTG | |
| | ATCCCAACAGACAACGGAACCAAAATCTACGGCCTGGAGTG | |
| | GAACGACGACTTCGTGACCGCCTACATCAGCGGCAGGTCCTA | |
| | CCATCTCAACATCAACACCAACTGGTTCAACAACGTCACCCT | |
| | CCTCTACAGCAGGTCATCCACAGCCACCTGGGAGTACTCAGC | |
| | TGCCTATGCATACCAGGGAGTCTCCAACTTCACATACTACAA | |
| | ACTCAACAACACCAACGGCCTCAAGACCTACGAGCTGTGTG | |
| | AGGACTACGAGCACTGCACCGGCTACGCAACAAACGTCTTC | |
| | GCCCCAACCTCCGGAGGCTACATCCCAGACGGCTTCTCCTTC | |
| | AACAACTGGTTCCTCCTCACAAACAGCTCCACCTTCGTGTCA | |
| | GGAAGGTTCGTGACCAACCAGCCCCTGCTCATCAACTGCCTC | |
| | TGGCCCGTCCCCTCCTTCGGAGTGGCCGCCCAGGAGTTCTGC | |
| | TTCGAGGGAGCCCAGTTCTCCCAGTGCAACGGAGTCTCCCTC | |
| | AACAACACCGTGGACGTCATCAGATTCAACCTCAACTTCACA | |
| | GCAGACGTCCAGAGCGGCATGGGAGCCACCGTGTTCAGCCT | |
| | GAACACCACAGGAGGAGTGATCCTGGAGATCTCCTGCTACTC | |
| | AGACACAGTGTCAGAGTCCTCCTCCTACAGCTACGGAGAGAT | |
| | CCCATTCGGCATCACAGACGGCCCCAGATACTGCTACGTGCT | |
| | GTACAACGGCACAGCCCTGAAGTACCTGGGCACCCTCCCCC | |
| | CATCAGTGAAGGAGATCGCCATCAGCAAGTGGGGCCACTTCT | |
| | ACATCAACGGCTACAACTTCTTCTCCACCTTCCCCATCGGCT | |
| | GCATCAGCTTCAACCTGACCACCGGAGTGTCCGGAGCCTTCT | |
| | GGACCATCGCCTACACATCATACACCGAGGCCCTGGTGCAGG | |
| | TGGAGAACACAGCCATAAAGAACGTGACCTACTGCAACAGC | |
| | CACATCAACAACATCAAGTGCTCCCAGCTGACAGCCAACCT | |
| | GAACAACGGCTTCTACCCAGTGGCCTCCAGCGAGGTGGGCT | |
| | TCGTGAACAAGAGCGTGGTCCTACTCCCCTCCTTCTTCACCT | |
| | ACACAGCAGTCAACATCACAATTGACCTGGGCATGAAGCTGT | |
| | CCGGCTACGGCCAGCCAATCGCCAGCACCCTGTCCAACATCA | |
| | CCCTGCCAATGCAGGACAACAACACCGACGTCTACTGCATCA | |
| | GAAGCAACCAGTTCTCCGTGTACGTCCACTCCACCTGCAAGT | |
| | CCTCCCTCTGGGACAACATCTTCAACCAGGACTGCACAGAC | |
| | GTGCTGGAGGCCACAGCTGTGATCAAGACAGGAACCTGCCC | |
| | TTTCTCATTCGACAAGCTCAACAACTACCTGACCTTCAACAA | |
| | GTTCTGCCTGAGCCTGTCCCCAGTGGGAGCCAACTGCAAGTT | |
| | CGACGTGGCCGCCAGAACCAGGACCAACGAGCAGGTGGTC | |
| | AGAAGCCTGTACGTCATCTACGAGGAGGGAGACAACATCGT | |
| | GGGAGTGCCCAGCGACAACTCAGGCCTGCACGACCTGAGCG | |
| | TGCTGCACCTGGACTCCTGCACAGACTACAACATCTACGGCA | |
| | GGACAGGAGTGGGCATCATCAGGAGGACCAACAGCACACTG | |
| | CTGTCCGGCCTCTACTACACCTCCCTGTCCGGAGACTTGCTG | |
| | GGATTCAAGAACGTGTCAGACGGAGTCATCTACAGCGTCACC | |
| | CCATGTGACGTGAGCGCCCAGGCAGCAGTGATAGACGGAGC | |
| | CATCGTGGGAGCCATGACCTCAATCAACTCAGAACTGCTGGG | |
| | CCTCACCCACTGGACAACAACACCCAACTTCTACTACTACTC | |
| | CATCTACAACTACACATCAGAAAGAACAAGGGACACAGCAA | |
| | TCGACTCCAACGACGTGGACTGTGAGCCAGTCATCACCTACT | |
| | CCAACATCGGCGTGTGCAAGAACGGAGCCCTGGTGTTCATC | |
| | AACGTCACCCACTCAGACGGCGACGTCCAGCCAATCTCCAC | |
| | AGGAAACGTCACCATCCCCACCAACTTCACCATCAGCGTGCA | |
| | GGTGGAGTACATGCAGGTCTACACCACCCCAGTCTCCATCGA | |
| | CTGTGCCAGGTACGTGTGCAACGGCAACCCAAGATGCAACA | |
| | AACTGCTGACCCAGTACGTGAGCGCCTGCCAGACCATCGAG | |
| | CAGGCCCTGGCCATGGGCGCCAGGCTGGAGAACATGGAGGT | |
| | GGACAGCATGCTCTTTGTGAGCGAGAACGCCCTGAAGCTTG | |
| | CCAGCGTGGAGGCCTTCAACAGCACCGAAAACCTGGACTCC | |
| | ATCTACAAAGAGTGGCCCTCCATAGGAGGCTCCTGGCTGGGA | |
| | GGCCTGAAGGACATCCTCCCATCCCACAACAGCAAAAGAAA | |
| | GTACGGCAGCGCCATCGAAGACCTGCTGTTCGACAAGGTGG | |
| | TCACCTCAGGACTGGGCACAGTGGACGAGGACTACAAGAGG | |
| | TGCACCGGAGGCTACGACATCGCAGACCTGGTCTGTGCCCA | |
| | GTACTACAACGGCATCATGGTGCTCCCAGGCGTGGCCAACGC | |
| | CGACAAGATGACCATGTACACAGCAAGCCTGGCTGGAGGAA | |
| | TCACACTGGGAGCCCTGGGAGGAGGGGCCGTGGCCATTCCA | |
| | TTCGCCGTGGCCGTGCAGGCCAGACTGAACTACGTGGCCCT | |
| | GCAGACAGACGTGCTAAACAAGAACCAGCAGATCCTGGCCA | |
| | ACGCCTTCAACCAGGCCATCGGCAACATCACCCAGGCCTTCG | |
| | GCAAGGTGAACGACGCAATCCACCAGACATCACAGGGCCTG | |
| | GCAACAGTGGCCAAGGCCCTGGCCAAGGTCCAGGACGTGGT | |
| | GAACACCCAGGGCCAGGCCCTCTCACACCTGACAGTCCAGC | |
| | TGCAGAACAACTTCCAGGCAATCTCCTCCTCCATCTCAGACA | |
| | TCTACAACAGACTGGACCCCCCCTCAGCCGACGCCCAGGTG | |
| | GACAGACTCATCACAGGCAGGCTGACCGCCCTCAACGCCTT | |
| | CGTGTCCCAGACCCTCACCAGGCAGGCCGAGGTGAGGGCCA | |
| | GCAGGCAGCTCGCCAAGGACAAGGTGAACGAGTGCGTCAG | |
| | AAGCCAGAGCCAGAGGTTCGGCTTCTGTGGCAACGGCACCC | |
| | ACCTGTTCTCCCTGGCCAACGCAGCCCCCAACGGCATGATCT | |
| | TCTTCCACACAGTCCTCCTCCCAACAGCATATGAGACAGTCA | |
| | CCGCCTGGTCAGGAATCTGTGCCTCAGACGGGGACAGAACC | |
| | TTCGGCCTGGTGGTCAAGGACGTGCAGCTGACACTCTTCAG | |
| | AAACCTGGACGACAAATTCTACCTGACCCCCAGGACCATGTA | |
| | CCAGCCAAGGGTGGCCACCTCCTCAGACTTCGTGCAGATCG | |
| | AGGGCTGTGACGTGCTCTTCGTGAACGCCACCGTCATCGACC | |
| | TCCCATCCATCATCCCAGACTACATCGACATCAACCAGACAG | |
| | TGCAGGACATCCTGGAGAACTACCGCCCCAACTGGACCGTG | |
| | CCAGAGTTCACCCTAGACATATTCAACGCCACCTACCTGAAC | |
| | CTGACAGGAGAAATTGACGACCTGGAGTTCAGATCAGAAAA | |
| | GCTACACAACACCACCGTGGAGTTAGCCATCCTCATAGACAA | |
| | CATTAACAACACCCTCGTCAACCTGGAGTGGCTCAACAGGAT | |
| | TGAAACCTACGTGAAGTGGCCC | |
| 165 | ATGACCACAAATAACGAGTGCATTCAGGTCAACGTCACCCAG | S_ec(S6) |
| | CTGGCCGGTAACGAGAACCTAATTAGAGACTTCCTATTCTCG | |
| | AACTTTAAAGAGGAAGGCTCTGTGGTGGTCGGAGGTTACTA | |
| | CCCCACAGAAGTGTGGTACAATTGCTCACGTACAGCCAGGA | |
| | CCACTGCCTTCCAGTACTTCAACAACATTCATGCCTTCTACTT | |
| | TGTCATGGAAGCCATGGAGAACTCCACTGGGAATGCCAGAG | |
| | GAAAGCCTCTCCTCTTCCATGTCCATGGAGAGCCTGTCTCTG | |
| | TGATTATCTCAGCATATAGGGATGATGTGCAGCAGCGGCCGCT | |
| | GCTTAAGCATGGCCTAGTGTGCATTACTAAGAACCGACATATC | |
| | AATTATGAGCAGTTCACCTCCAACCAGTGGAACTCCACATGC | |
| | ACTGGTGCTGATAGGAAGATCCCGTTCAGCGTTATCCCCACC | |
| | GATAATGGCACAAAGATTTATGGCCTAGAATGGAACGATGAT | |
| | TTTGTTACTGCCTACATATCAGGAAGAAGTTACCACTTAAACA | |
| | TTAACACCAATTGGTTCAATAATGTTACACTTCTGTACTCTCG | |
| | CAGCAGTACGGCCACTTGGGAGTATTCGGCTGCATATGCCTA | |
| | CCAAGGTGTAAGCAACTTCACCTACTACAAGCTGAACAATAC | |
| | GAACGGTCTGAAGACTTATGAGCTGTGCGAAGACTACGAGC | |
| | ACTGTACGGGCTATGCGACAAATGTCTTCGCCCCGACGAGCG | |
| | GCGGGTACATACCGGATGGCTTCTCCTTCAACAACTGGTTCC | |
| | TCCTTACCAATAGCTCCACTTTCGTATCAGGAAGATTTGTTAC | |
| | GAACCAACCCCTTCTCATTAACTGTCTGTGGCCAGTGCCCTC | |
| | CTTCGGAGTAGCTGCTCAAGAGTTCTGTTTCGAGGGTGCACA | |
| | GTTCAGCCAGTGTAATGGAGTGTCGCTGAACAACACTGTGG | |
| | ACGTGATCAGGTTTAATTTGAACTTCACAGCTGATGTTCAGTC | |
| | CGGCATGGGCGCGACTGTGTTCAGCCTAAACACCACGGGTG | |
| | GCGTCATCTTGGAGATTAGTTGTTACTCTGACACTGTGTCAG | |
| | AGAGCAGCAGTTACTCCTACGGAGAAATTCCTTTCGGCATCA | |
| | CAGACGGTCCCCGGTACTGCTATGTGCTGTACAACGGAACTG | |
| | CTTTGAAGTACCTGGGGACATTGCCACCTTCTGTGAAGGAAA | |
| | TAGCCATCTCTAAGTGGGGTCACTTTTACATTAACGGCTATAA | |
| | TTTCTTTTCCACTTTCCCAATTGGATGCATTAGCTTCAACCTG | |
| | ACAACAGGTGTGTCTGGAGCCTTCTGGACCATCGCCTATACC | |
| | TCTTACACAGAGGCTCTAGTACAGGTGGAGAACACAGCTATA | |
| | AAGAACGTGACGTACTGTAACAGTCACATAAACAATATCAAG | |
| | TGTTCTCAGTTGACTGCGAACTTAAACAATGGGTTTTATCCA | |
| | GTGGCGAGCTCGGAGGTGGGGTTTGTAAACAAATCTGTGGT | |
| | GCTGTTGCCCTCCTTCTTCACGTACACTGCAGTGAACATCAC | |
| | CATTGATTTGGGGATGAAACTGTCCGGCTACGGGCAGCCTAT | |
| | AGCATCTACACTGAGCAATATCACACTGCCCATGCAGGATAA | |
| | CAATACAGATGTGTACTGTATCCGCTCAAACCAGTTCTCTGTA | |
| | TACGTGCACAGTACATGCAAGAGCTCGCTATGGGACAACATT | |
| | TTCAACCAGGATTGTACTGATGTGCTTGAAGCAACTGCAGTG | |
| | ATCAAAACAGGCACATGCCCGTTCAGCTTTGATAAGCTCAAC | |
| | AACTACCTAACGTTCAACAAGTTCTGCTTGAGCCTGTCTCCA | |
| | GTAGGCGCCAATTGCAAGTTTGACGTTGCAGCGCGAACACG | |
| | GACAAACGAACAGGTAGTGCGGTCGCTCTATGTTATCTACGA | |
| | GGAGGGGGACAACATAGTCGGGGTTCCATCCGACAACTCAG | |
| | GTTTGCACGACCTGAGTGTGCTCCATTTGGACTCATGCACGG | |
| | ATTATAACATCTACGGGCGCACAGGTGTGGGGATAATACGAA | |
| | GAACAAACTCTACGCTATTGAGCGGGCTCTACTACACCTCAT | |
| | TGAGTGGGGACCTGCTAGGGTTCAAGAACGTATCTGACGGT | |
| | GTGATCTATAGCGTCACACCATGTGACGTATCAGCCCAAGCT | |
| | GCTGTGATTGACGGGGCGATTGTGGGGGCTATGACTTCAATT | |
| | AACAGCGAGCTCCTAGGCCTGACCCACTGGACTACCACCCC | |
| | AAACTTCTACTACTACAGCATTTATAACTATACCAGTGAGCGC | |
| | ACCAGGGACACTGCCATTGACAGCAATGACGTCGACTGCGA | |
| | GCCTGTTATTACCTACAGCAACATCGGTGTTTGTAAGAATGGA | |
| | GCTCTAGTCTTCATAAACGTAACGCACTCTGATGGCGATGTTC | |
| | AACCAATTTCCACTGGGAACGTAACCATACCCACCAACTTTA | |
| | CTATTTCCGTCCAGGTGGAGTACATGCAAGTATATACCACGCC | |
| | AGTGTCCATCGACTGCGCTCGGTATGTGTGCAACGGTAACCC | |
| | ACGCTGCAATAAGCTGCTAACGCAGTACGTCAGCGCCTGCCA | |
| | GACAATAGAGCAGGCATTGGCAATGGGTGCAAGGCTTGAAA | |
| | ACATGGAGGTGGACTCCATGTTGTTCGTGTCTGAAAACGCTC | |
| | TTAAACTAGCATCCGTGGAGGCATTCAACAGTACTGAGAACT | |
| | TGGACTCTATCTATAAGGAGTGGCCCTCCATTGGGGGCAGCT | |
| | GGCTTGGAGGTCTAAAAGACATCCTGCCCAGCCACAACTCC | |
| | AAGAGGAAGTACGGGTCCGCTATAGAGGACCTCCTCTTTGAC | |
| | AAGGTTGTTACTTCTGGTCTTGGCACAGTGGACGAAGACTAC | |
| | AAGAGGTGCACAGGAGGCTATGATATAGCTGACCTGGTGTGT | |
| | GCTCAATACTACAACGGTATAATGGTTCTCCCAGGTGTGGCC | |
| | AACGCTGACAAGATGACAATGTACACAGCCTCTTTAGCTGGA | |
| | GGCATTACCCTGGGAGCCCTTGGGGGTGGCGCAGTGGCAATT | |
| | CCATTTGCCGTTGCGGTGCAGGCCCGACTAAACTATGTCGCA | |
| | CTTCAAACAGATGTGCTCAACAAGAACCAACAAATACTGGC | |
| | CAACGCTTTCAACCAGGCCATTGGTAACATTACGCAGGCATT | |
| | TGGCAAGGTGAATGACGCCATCCACCAGACCAGCCAGGGAC | |
| | TTGCCACAGTGGCCAAGGCCTTGGCAAAGGTGCAGGATGTC | |
| | GTGAACACACAGGGTCAGGCCCTCTCTCATTTGACAGTGCA | |
| | GCTTCAGAATAACTTCCAAGCAATCAGTTCAAGCATCAGCGA | |
| | CATCTACAACCGGCTGGACCCCCCATCTGCAGATGCGCAGGT | |
| | GGACAGGCTAATCACTGGACGCTTGACGGCACTAAATGCCTT | |
| | TGTCAGCCAAACTCTGACCCGGCAAGCAGAGGTGCGGGCCA | |
| | GTAGACAACTGGCCAAAGACAAGGTCAACGAGTGCGTCAGG | |
| | TCCCAGTCCCAGCGTTTTGGATTCTGTGGGAACGGGACGCAC | |
| | CTGTTCTCATTAGCCAATGCTGCACCCAATGGCATGATCTTTT | |
| | TCCATACTGTTCTACTTCCTACTGCCTATGAAACCGTGACCGC | |
| | TTGGAGCGGCATCTGCGCATCTGATGGCGATAGGACCTTCGG | |
| | GCTGGTCGTTAAGGATGTCCAGCTAACGCTGTTCCGGAACTT | |
| | GGATGACAAGTTCTACCTGACCCCCAGGACCATGTACCAGCC | |
| | GAGAGTGGCAACGAGTTCTGACTTCGTGCAAATTGAGGGCT | |
| | GTGACGTCCTGTTTGTTAATGCAACAGTGATCGATCTGCCCA | |
| | GTATCATACCAGATTACATAGACATAAACCAGACAGTCCAGG | |
| | ACATACTGGAGAATTACAGGCCAAACTGGACCGTACCAGAG | |
| | TTCACGCTGGACATATTCAACGCTACGTACCTCAATTTGACTG | |
| | GGGAAATTGATGACTTGGAGTTCAGGTCGGAGAAGCTCCAC | |
| | AACACCACTGTGGAGCTGGCCATCCTGATTGACAACATCAAC | |
| | AACACTCTGGTGAACCTGGAGTGGCTAAATCGCATTGAAACC | |
| | TATGTCAAGTGGCCT | |
| 166 | ATGTGTGAGCCAGTCATCACCTACAGCAACATCGGAGTGTGC | SII(S2) |
| | AAGAACGGAGCCCTGGTGTTCATCAACGTGACCCACAGCGA | |
| | CGGAGATGTCCAGCCCATCAGCACAGGAAATGTGACCATCCC | |
| | AACCAACTTCACCATCAGCGTCCAGGTGGAATACATGCAGGT | |
| | GTACACCACCCCAGTGTCCATCGACTGTGCCAGATACGTGTG | |
| | CAATGGAAACCCCAGATGCAACAAGCTCCTCACCCAGTACG | |
| | TGTCAGCCTGCCAGACAATCGAGCAGGCCCTGGCCATGGGA | |
| | GCCAGGCTCGAGAACATGGAAGTGGACAGCATGCTGTTTGT | |
| | CTCAGAGAATGCCCTGAAACTGGCCAGCGTGGAGGCCTTCA | |
| | ACAGCACAGAGAACCTGGACAGCATCTACAAGGAGTGGCCA | |
| | TCAATCGGAGGCAGCTGGCTGGGAGGACTTAAGGACATCCT | |
| | GCCAAGCCACAACAGCAAAAGAAAGTACGGCAGCGCCATTG | |
| | AGGACCTGCTGTTTGACAAGGTGGTCACCTCCGGCCTGGGC | |
| | ACAGTGGATGAGGACTACAAGAGATGCACCGGCGGCTATGA | |
| | CATTGCCGACCTGGTGTGTGCCCAGTACTACAATGGCATCAT | |
| | GGTGCTGCCTGGAGTGGCCAACGCCGACAAAATGACCATGT | |
| | ACACCGCCTCCCTGGCTGGAGGCATCACACTGGGAGCCCTG | |
| | GGGGGAGGAGCAGTGGCCATCCCCTTTGCAGTGGCTGTGCA | |
| | GGCCAGACTCAACTACGTGGCCCTGCAGACAGACGTGCTCA | |
| | ACAAGAACCAGCAGATCCTGGCCAACGCTTTCAACCAGGCT | |
| | ATCGGAAACATCACCCAGGCCTTTGGAAAAGTGAATGATGCC | |
| | ATCCACCAGACCAGCCAGGGCCTGGCCACAGTGGCCAAGGC | |
| | CCTGGCCAAGGTGCAGGACGTGGTCAACACCCAGGGCCAGG | |
| | CCCTCAGTCACCTCACAGTACAGCTCCAGAACAACTTCCAG | |
| | GCAATCTCCTCCTCCATCAGCGACATCTACAACAGGCTGGAC | |
| | CCCCCAAGCGCTGATGCCCAGGTGGACAGACTGATCACAGG | |
| | AAGACTCACAGCCCTCAACGCATTTGTGTCCCAGACACTGAC | |
| | CAGGCAGGCAGAGGTCAGGGCCAGCAGGCAGCTGGCCAAG | |
| | GACAAGGTGAATGAGTGCGTGAGGAGCCAGAGCCAGAGATT | |
| | TGGCTTCTGCGGAAACGGCACCCACCTGTTCAGCCTGGCCA | |
| | ACGCCGCCCCCAACGGCATGATTTTCTTCCACACAGTCCTCC | |
| | TCCCCACAGCCTACGAAACAGTGACAGCCTGGTCAGGCATCT | |
| | GTGCCAGCGACGGAGACAGAACCTTTGGCCTGGTGGTGAAG | |
| | GATGTGCAGCTCACCCTCTTCAGAAACCTGGATGACAAGTTC | |
| | TACCTCACCCCAAGAACCATGTACCAGCCCAGAGTGGCCAC | |
| | AAGCAGCGACTTTGTGCAGATTGAGGGCTGTGACGTGCTGTT | |
| | TGTGAATGCAACAGTGATTGACCTCCCAAGCATCATCCCAGA | |
| | TTACATCGACATCAACCAGACAGTGCAGGACATCCTGGAGAA | |
| | CTACAGGCCCAACTGGACAGTGCCAGAGTTCACCCTGGACA | |
| | TCTTCAACGCCACCTACCTGAACCTGACAGGAGAAATTGACG | |
| | ACCTGGAGTTCAGATCAGAAAAACTTCACAACACCACCGTG | |
| | GAGCTTGCCATCCTCATTGACAACATTAACAACACACTGGTC | |
| | AACCTGGAATGGCTGAACAGAATTGAAACCTACGTGAAGTG | |
| | GCCCTGGTATGTGTGGCTGCTGATTGGACTGGTGGTGGTGTT | |
| | CTGCATCCCACTGCTGCTGTTCTGCTGCTTCAGCACCGGCTG | |
| | CTGTGGATGCATCGGCTGCTTGGGCAGCTGCTGCCACAGCAT | |
| | CTGCAGCAGGAGGCAGTTTGAGAACTACGAACCAATTGAAA | |
| | AAGTGCACGTCCAC | |
| 167 | ATGTGTGAGCCAGTCATCACCTACTCCAACATCGGCGTGTGC | SII(S3) |
| | AAGAACGGAGCCCTGGTGTTCATCAACGTCACCCACTCAGA | |
| | CGGCGACGTCCAGCCAATCTCCACAGGAAACGTCACCATCC | |
| | CCACCAACTTCACCATCAGCGTGCAGGTGGAGTACATGCAGG | |
| | TCTACACCACCCCAGTCTCCATCGACTGTGCCAGGTACGTGT | |
| | GCAACGGCAACCCAAGATGCAACAAACTGCTGACCCAGTAC | |
| | GTGAGCGCCTGCCAGACCATCGAGCAGGCCCTGGCCATGGG | |
| | CGCCAGGCTGGAGAACATGGAGGTGGACAGCATGCTCTTTG | |
| | TGAGCGAGAACGCCCTGAAGCTTGCCAGCGTGGAGGCCTTC | |
| | AACAGCACCGAAAACCTGGACTCCATCTACAAAGAGTGGCC | |
| | CTCCATAGGAGGCTCCTGGCTGGGAGGCCTGAAGGACATCCT | |
| | CCCATCCCACAACAGCAAAAGAAAGTACGGCAGCGCCATCG | |
| | AAGACCTGCTGTTCGACAAGGTGGTCACCTCAGGACTGGGC | |
| | ACAGTGGACGAGGACTACAAGAGGTGCACCGGAGGCTACGA | |
| | CATCGCAGACCTGGTCTGTGCCCAGTACTACAACGGCATCAT | |
| | GGTGCTCCCAGGCGTGGCCAACGCCGACAAGATGACCATGT | |
| | ACACAGCAAGCCTGGCTGGAGGAATCACACTGGGAGCCCTG | |
| | GGAGGAGGGGCCGTGGCCATTCCATTCGCCGTGGCCGTGCA | |
| | GGCCAGACTGAACTACGTGGCCCTGCAGACAGACGTGCTAA | |
| | ACAAGAACCAGCAGATCCTGGCCAACGCCTTCAACCAGGCC | |
| | ATCGGCAACATCACCCAGGCCTTCGGCAAGGTGAACGACGC | |
| | AATCCACCAGACATCACAGGGCCTGGCAACAGTGGCCAAGG | |
| | CCCTGGCCAAGGTCCAGGACGTGGTGAACACCCAGGGCCAG | |
| | GCCCTCTCACACCTGACAGTCCAGCTGCAGAACAACTTCCA | |
| | GGCAATCTCCTCCTCCATCTCAGACATCTACAACAGACTGGA | |
| | CCCCCCCTCAGCCGACGCCCAGGTGGACAGACTCATCACAG | |
| | GCAGGCTGACCGCCCTCAACGCCTTCGTGTCCCAGACCCTCA | |
| | CCAGGCAGGCCGAGGTGAGGGCCAGCAGGCAGCTCGCCAA | |
| | GGACAAGGTGAACGAGTGCGTCAGAAGCCAGAGCCAGAGG | |
| | TTCGGCTTCTGTGGCAACGGCACCCACCTGTTCTCCCTGGCC | |
| | AACGCAGCCCCCAACGGCATGATCTTCTTCCACACAGTCCTC | |
| | CTCCCAACAGCATATGAGACAGTCACCGCCTGGTCAGGAATC | |
| | TGTGCCTCAGACGGGGACAGAACCTTCGGCCTGGTGGTCAA | |
| | GGACGTGCAGCTGACACTCTTCAGAAACCTGGACGACAAAT | |
| | TCTACCTGACCCCCAGGACCATGTACCAGCCAAGGGTGGCCA | |
| | CCTCCTCAGACTTCGTGCAGATCGAGGGCTGTGACGTGCTCT | |
| | TCGTGAACGCCACCGTCATCGACCTCCCATCCATCATCCCAG | |
| | ACTACATCGACATCAACCAGACAGTGCAGGACATCCTGGAG | |
| | AACTACCGCCCCAACTGGACCGTGCCAGAGTTCACCCTAGA | |
| | CATATTCAACGCCACCTACCTGAACCTGACAGGAGAAATTGA | |
| | CGACCTGGAGTTCAGATCAGAAAAGCTACACAACACCACCG | |
| | TGGAGTTAGCCATCCTCATAGACAACATTAACAACACCCTCG | |
| | TCAACCTGGAGTGGCTCAACAGGATTGAAACCTACGTGAAG | |
| | TGGCCCTGGTACGTCTGGCTCCTCATCGGCCTGGTGGTGGTC | |
| | TTCTGCATCCCACTGCTGCTGTTCTGCTGCTTCTCCACCGGCT | |
| | GCTGTGGATGCATCGGCTGCCTGGGCTCATGCTGCCACTCAA | |
| | TCTGCTCAAGGAGGCAGTTTGAAAACTACGAGCCAATAGAA | |
| | AAAGTCCACGTCCAC | |
| 168 | ATGTGCGAGCCTGTTATTACCTACAGCAACATCGGTGTTTGTA | SII(S6) |
| | AGAATGGAGCTCTAGTCTTCATAAACGTAACGCACTCTGATG | |
| | GCGATGTTCAACCAATTTCCACTGGGAACGTAACCATACCCA | |
| | CCAACTTTACTATTTCCGTCCAGGTGGAGTACATGCAAGTATA | |
| | TACCACGCCAGTGTCCATCGACTGCGCTCGGTATGTGTGCAA | |
| | CGGTAACCCACGCTGCAATAAGCTGCTAACGCAGTACGTCAG | |
| | CGCCTGCCAGACAATAGAGCAGGCATTGGCAATGGGTGCAA | |
| | GGCTTGAAAACATGGAGGTGGACTCCATGTTGTTCGTGTCTG | |
| | AAAACGCTCTTAAACTAGCATCCGTGGAGGCATTCAACAGTA | |
| | CTGAGAACTTGGACTCTATCTATAAGGAGTGGCCCTCCATTG | |
| | GGGGCAGCTGGCTTGGAGGTCTAAAAGACATCCTGCCCAGC | |
| | CACAACTCCAAGAGGAAGTACGGGTCCGCTATAGAGGACCT | |
| | CCTCTTTGACAAGGTTGTTACTTCTGGTCTTGGCACAGTGGA | |
| | CGAAGACTACAAGAGGTGCACAGGAGGCTATGATATAGCTGA | |
| | CCTGGTGTGTGCTCAATACTACAACGGTATAATGGTTCTCCCA | |
| | GGTGTGGCCAACGCTGACAAGATGACAATGTACACAGCCTCT | |
| | TTAGCTGGAGGCATTACCCTGGGAGCCCTTGGGGGTGGCGCA | |
| | GTGGCAATTCCATTTGCCGTTGCGGTGCAGGCCCGACTAAAC | |
| | TATGTCGCACTTCAAACAGATGTGCTCAACAAGAACCAACAA | |
| | ATACTGGCCAACGCTTTCAACCAGGCCATTGGTAACATTACG | |
| | CAGGCATTTGGCAAGGTGAATGACGCCATCCACCAGACCAG | |
| | CCAGGGACTTGCCACAGTGGCCAAGGCCTTGGCAAAGGTGC | |
| | AGGATGTCGTGAACACACAGGGTCAGGCCCTCTCTCATTTGA | |
| | CAGTGCAGCTTCAGAATAACTTCCAAGCAATCAGTTCAAGCA | |
| | TCAGCGACATCTACAACCGGCTGGACCCCCCATCTGCAGATG | |
| | CGCAGGTGGACAGGCTAATCACTGGACGCTTGACGGCACTA | |
| | AATGCCTTTGTCAGCCAAACTCTGACCCGGCAAGCAGAGGT | |
| | GCGGGCCAGTAGACAACTGGCCAAAGACAAGGTCAACGAGT | |
| | GCGTCAGGTCCCAGTCCCAGCGTTTTGGATTCTGTGGGAACG | |
| | GGACGCACCTGTTCTCATTAGCCAATGCTGCACCCAATGGCA | |
| | TGATCTTTTTCCATACTGTTCTACTTCCTACTGCCTATGAAACC | |
| | GTGACCGCTTGGAGCGGCATCTGCGCATCTGATGGCGATAGG | |
| | ACCTTCGGGCTGGTCGTTAAGGATGTCCAGCTAACGCTGTTC | |
| | CGGAACTTGGATGACAAGTTCTACCTGACCCCCAGGACCATG | |
| | TACCAGCCGAGAGTGGCAACGAGTTCTGACTTCGTGCAAATT | |
| | GAGGGCTGTGACGTCCTGTTTGTTAATGCAACAGTGATCGAT | |
| | CTGCCCAGTATCATACCAGATTACATAGACATAAACCAGACA | |
| | GTCCAGGACATACTGGAGAATTACAGGCCAAACTGGACCGT | |
| | ACCAGAGTTCACGCTGGACATATTCAACGCTACGTACCTCAA | |
| | TTTGACTGGGGAAATTGATGACTTGGAGTTCAGGTCGGAGA | |
| | AGCTCCACAACACCACTGTGGAGCTGGCCATCCTGATTGACA | |
| | ACATCAACAACACTCTGGTGAACCTGGAGTGGCTAAATCGCA | |
| | TTGAAACCTATGTCAAGTGGCCTTGGTACGTTTGGCTACTGAT | |
| | CGGACTCGTGGTAGTCTTCTGCATACCACTCCTGCTATTTTGC | |
| | TGCTTCAGCACAGGGTGCTGTGGCTGCATTGGATGCCTAGGT | |
| | TCCTGCTGTCACAGTATCTGCAGCAGAAGACAATTCGAGAAC | |
| | TACGAGCCCATAGAAAAGGTCCACGTACAT | |
| 169 | ATGAGATTTGTGATGTCCCCCACTGTCCTGCTGCTGCTCCTGG | MHCIsp- |
| | GAGCCCTGGCAGCCCCCCAGACCTGGGCAGGCAGCACCACC | S(S2) |
| | AACAATGAATGCATCCAGGTGAACGTGACCCAGCTGGCAGG | |
| | CAATGAAAATTTGATCAGAGACTTCCTGTTCAGCAACTTCAA | |
| | GGAGGAGGGCAGTGTAGTGGTGGGAGGCTACTACCCAACAG | |
| | AGGTGTGGTACAACTGCAGCAGAACAGCCAGAACCACAGCC | |
| | TTCCAGTACTTCAACAACATCCACGCCTTCTACTTTGTGATGG | |
| | AGGCCATGGAAAACAGCACAGGAAATGCCAGAGGAAAACC | |
| | CCTGCTCTTCCACGTGCACGGAGAGCCCGTGTCAGTCATCAT | |
| | CAGCGCCTACAGAGATGACGTCCAGCAGCGGCCCCTGCTGA | |
| | AGCATGGACTGGTCTGCATCACCAAGAACAGACACATCAAC | |
| | TACGAGCAGTTCACCAGCAACCAGTGGAACAGCACCTGCAC | |
| | AGGAGCAGACAGAAAAATCCCCTTCAGCGTCATCCCCACAG | |
| | ACAACGGCACCAAAATCTATGGCCTGGAGTGGAATGATGACT | |
| | TTGTGACAGCCTATATCAGCGGCAGGAGCTACCACCTCAACA | |
| | TCAACACCAACTGGTTCAACAACGTCACCCTGCTCTACTCCA | |
| | GATCCAGCACAGCCACCTGGGAGTACAGCGCCGCCTATGCCT | |
| | ACCAGGGAGTCTCCAACTTCACCTACTACAAACTGAACAAC | |
| | ACCAACGGCCTGAAAACCTACGAGCTGTGTGAGGACTACGA | |
| | GCACTGCACAGGCTATGCCACAAATGTGTTTGCCCCAACCAG | |
| | CGGAGGCTACATCCCAGACGGCTTCTCCTTCAACAACTGGTT | |
| | CCTCCTCACCAACTCCTCCACATTTGTGAGCGGCAGATTTGT | |
| | GACCAACCAGCCCCTGCTGATCAACTGCCTGTGGCCCGTGCC | |
| | CAGCTTTGGAGTGGCAGCCCAGGAGTTCTGCTTCGAGGGAG | |
| | CCCAGTTCAGCCAGTGCAACGGAGTCAGCCTGAACAACACA | |
| | GTGGACGTGATCAGATTCAACCTGAACTTCACAGCAGACGT | |
| | GCAGAGTGGAATGGGAGCCACCGTCTTCAGCCTGAACACCA | |
| | CAGGAGGAGTGATCCTGGAGATCAGCTGCTACAGCGACACA | |
| | GTGAGCGAGAGCAGCAGCTACAGCTACGGAGAGATCCCATT | |
| | TGGCATCACAGATGGCCCCAGGTACTGCTACGTCCTGTACAA | |
| | TGGAACAGCCCTGAAATACCTGGGCACCCTCCCACCCAGCG | |
| | TGAAGGAGATCGCCATCAGCAAGTGGGGCCACTTCTACATCA | |
| | ATGGCTACAACTTCTTCAGCACCTTCCCCATCGGCTGCATCTC | |
| | CTTCAACCTGACCACAGGAGTGAGCGGGGCCTTCTGGACAA | |
| | TCGCCTACACATCCTACACAGAAGCCCTGGTGCAGGTGGAG | |
| | AACACAGCCATCAAAAACGTCACCTACTGCAACAGCCACAT | |
| | CAACAACATCAAGTGCAGCCAGCTGACAGCCAACCTGAACA | |
| | ACGGCTTCTACCCAGTGGCCAGCTCAGAGGTGGGCTTCGTG | |
| | AACAAGAGCGTGGTGCTCCTGCCCAGCTTCTTCACCTACACA | |
| | GCAGTGAACATCACAATTGACCTGGGCATGAAGCTGAGCGG | |
| | CTACGGCCAGCCAATTGCCAGCACCCTCTCCAACATCACCCT | |
| | CCCCATGCAGGACAATAACACAGATGTGTACTGCATCAGATC | |
| | CAACCAGTTCTCTGTCTACGTGCACAGCACCTGCAAAAGCA | |
| | GCCTGTGGGACAACATCTTCAACCAGGACTGCACAGATGTCC | |
| | TGGAGGCCACAGCCGTGATCAAAACAGGCACCTGCCCCTTC | |
| | AGCTTTGACAAACTCAACAACTACCTTACATTCAACAAATTC | |
| | TGCCTCTCCCTCAGCCCAGTGGGAGCCAACTGCAAGTTTGAT | |
| | GTGGCCGCCAGGACCAGGACAAATGAACAAGTGGTCAGAA | |
| | GCCTCTACGTCATCTACGAGGAGGGAGACAACATCGTGGGG | |
| | GTCCCCAGCGACAACAGCGGCCTGCACGACCTGAGTGTGCT | |
| | CCACCTGGACAGCTGCACAGACTACAACATCTACGGCAGGA | |
| | CTGGGGTGGGCATCATCAGAAGAACCAACAGCACACTGCTG | |
| | AGTGGCCTGTACTACACCAGCCTGAGTGGAGACTTGCTGGG | |
| | CTTCAAGAATGTGTCAGATGGGGTGATCTACAGTGTGACCCC | |
| | CTGTGACGTGTCTGCCCAGGCTGCAGTCATCGACGGAGCCAT | |
| | CGTGGGAGCCATGACCAGCATTAACAGCGAGCTGCTGGGCC | |
| | TGACCCACTGGACCACCACCCCCAACTTCTACTACTACTCCA | |
| | TCTACAACTACACATCAGAAAGAACAAGAGACACAGCCATC | |
| | GACAGCAATGACGTGGACTGTGAGCCAGTCATCACCTACAG | |
| | CAACATCGGAGTGTGCAAGAACGGAGCCCTGGTGTTCATCA | |
| | ACGTGACCCACAGCGACGGAGATGTCCAGCCCATCAGCACA | |
| | GGAAATGTGACCATCCCAACCAACTTCACCATCAGCGTCCAG | |
| | GTGGAATACATGCAGGTGTACACCACCCCAGTGTCCATCGAC | |
| | TGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCCTGGTATGTGTGGCTGCTGATTGG | |
| | ACTGGTGGTGGTGTTCTGCATCCCACTGCTGCTGTTCTGCTG | |
| | CTTCAGCACCGGCTGCTGTGGATGCATCGGCTGCTTGGGCAG | |
| | CTGCTGCCACAGCATCTGCAGCAGGAGGCAGTTTGAGAACT | |
| | ACGAACCAATTGAAAAAGTGCACGTCCAC | |
| 170 | ATGAGATTTGTGATGAGCCCCACTGTGCTGCTGCTGCTGCTG | MHCIsp- |
| | GGAGCCCTGGCAGCCCCCCAGACCTGGGCTGGCTCAACCAC | S(S3) |
| | CAACAACGAGTGCATCCAGGTGAACGTGACCCAGCTGGCAG | |
| | GCAACGAGAACCTCATCAGAGACTTCCTCTTCTCCAACTTCA | |
| | AGGAGGAGGGCTCAGTGGTGGTCGGCGGCTACTACCCAACA | |
| | GAGGTGTGGTACAACTGCTCAAGGACCGCCAGAACCACAGC | |
| | CTTCCAGTACTTCAACAACATCCACGCCTTCTACTTCGTGATG | |
| | GAGGCCATGGAGAACTCCACCGGGAACGCCAGGGGCAAGC | |
| | CACTACTCTTCCACGTGCACGGAGAGCCAGTGAGCGTGATCA | |
| | TCTCAGCCTACAGGGACGACGTGCAGCAGCGCCCCCTGCTG | |
| | AAGCATGGACTGGTGTGCATCACCAAGAACAGGCACATCAA | |
| | CTACGAGCAGTTCACCAGCAACCAGTGGAACAGCACCTGCA | |
| | CCGGCGCAGACAGGAAGATCCCCTTCTCAGTGATCCCAACA | |
| | GACAACGGAACCAAAATCTACGGCCTGGAGTGGAACGACGA | |
| | CTTCGTGACCGCCTACATCAGCGGCAGGTCCTACCATCTCAA | |
| | CATCAACACCAACTGGTTCAACAACGTCACCCTCCTCTACAG | |
| | CAGGTCATCCACAGCCACCTGGGAGTACTCAGCTGCCTATGC | |
| | ATACCAGGGAGTCTCCAACTTCACATACTACAAACTCAACAA | |
| | CACCAACGGCCTCAAGACCTACGAGCTGTGTGAGGACTACG | |
| | AGCACTGCACCGGCTACGCAACAAACGTCTTCGCCCCAACC | |
| | TCCGGAGGCTACATCCCAGACGGCTTCTCCTTCAACAACTGG | |
| | TTCCTCCTCACAAACAGCTCCACCTTCGTGTCAGGAAGGTTC | |
| | GTGACCAACCAGCCCCTGCTCATCAACTGCCTCTGGCCCGTC | |
| | CCCTCCTTCGGAGTGGCCGCCCAGGAGTTCTGCTTCGAGGG | |
| | AGCCCAGTTCTCCCAGTGCAACGGAGTCTCCCTCAACAACA | |
| | CCGTGGACGTCATCAGATTCAACCTCAACTTCACAGCAGACG | |
| | TCCAGAGCGGCATGGGAGCCACCGTGTTCAGCCTGAACACC | |
| | ACAGGAGGAGTGATCCTGGAGATCTCCTGCTACTCAGACACA | |
| | GTGTCAGAGTCCTCCTCCTACAGCTACGGAGAGATCCCATTC | |
| | GGCATCACAGACGGCCCCAGATACTGCTACGTGCTGTACAAC | |
| | GGCACAGCCCTGAAGTACCTGGGCACCCTCCCCCCATCAGTG | |
| | AAGGAGATCGCCATCAGCAAGTGGGGCCACTTCTACATCAAC | |
| | GGCTACAACTTCTTCTCCACCTTCCCCATCGGCTGCATCAGCT | |
| | TCAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCCTGGTACGTCTGGCTCCTCATCGGCC | |
| | TGGTGGTGGTCTTCTGCATCCCACTGCTGCTGTTCTGCTGCTT | |
| | CTCCACCGGCTGCTGTGGATGCATCGGCTGCCTGGGCTCATG | |
| | CTGCCACTCAATCTGCTCAAGGAGGCAGTTTGAAAACTACGA | |
| | GCCAATAGAAAAAGTCCACGTCCAC | |
| 171 | ATGCGTTTTGTAATGTCACCTACTGTACTACTACTACTACTCG | MHCIsp- |
| | GAGCACTAGCAGCACCTCAGACTTGGGCCGGATCAACCACA | S(S6) |
| | AATAACGAGTGCATTCAGGTCAACGTCACCCAGCTGGCCGGT | |
| | AACGAGAACCTAATTAGAGACTTCCTATTCTCGAACTTTAAA | |
| | GAGGAAGGCTCTGTGGTGGTCGGAGGTTACTACCCCACAGA | |
| | AGTGTGGTACAATTGCTCACGTACAGCCAGGACCACTGCCTT | |
| | CCAGTACTTCAACAACATTCATGCCTTCTACTTTGTCATGGAA | |
| | GCCATGGAGAACTCCACTGGGAATGCCAGAGGAAAGCCTCT | |
| | CCTCTTCCATGTCCATGGAGAGCCTGTCTCTGTGATTATCTCA | |
| | GCATATAGGGATGATGTGCAGCAGCGGCCGCTGCTTAAGCAT | |
| | GGCCTAGTGTGCATTACTAAGAACCGACATATCAATTATGAGC | |
| | AGTTCACCTCCAACCAGTGGAACTCCACATGCACTGGTGCTG | |
| | ATAGGAAGATCCCGTTCAGCGTTATCCCCACCGATAATGGCA | |
| | CAAAGATTTATGGCCTAGAATGGAACGATGATTTTGTTACTGC | |
| | CTACATATCAGGAAGAAGTTACCACTTAAACATTAACACCAA | |
| | TTGGTTCAATAATGTTACACTTCTGTACTCTCGCAGCAGTACG | |
| | GCCACTTGGGAGTATTCGGCTGCATATGCCTACCAAGGTGTA | |
| | AGCAACTTCACCTACTACAAGCTGAACAATACGAACGGTCTG | |
| | AAGACTTATGAGCTGTGCGAAGACTACGAGCACTGTACGGG | |
| | CTATGCGACAAATGTCTTCGCCCCGACGAGCGGCGGGTACAT | |
| | ACCGGATGGCTTCTCCTTCAACAACTGGTTCCTCCTTACCAAT | |
| | AGCTCCACTTTCGTATCAGGAAGATTTGTTACGAACCAACCC | |
| | CTTCTCATTAACTGTCTGTGGCCAGTGCCCTCCTTCGGAGTAG | |
| | CTGCTCAAGAGTTCTGTTTCGAGGGTGCACAGTTCAGCCAGT | |
| | GTAATGGAGTGTCGCTGAACAACACTGTGGACGTGATCAGGT | |
| | TTAATTTGAACTTCACAGCTGATGTTCAGTCCGGCATGGGCG | |
| | CGACTGTGTTCAGCCTAAACACCACGGGTGGCGTCATCTTGG | |
| | AGATTAGTTGTTACTCTGACACTGTGTCAGAGAGCAGCAGTT | |
| | ACTCCTACGGAGAAATTCCTTTCGGCATCACAGACGGTCCCC | |
| | GGTACTGCTATGTGCTGTACAACGGAACTGCTTTGAAGTACC | |
| | TGGGGACATTGCCACCTTCTGTGAAGGAAATAGCCATCTCTA | |
| | AGTGGGGTCACTTTTACATTAACGGCTATAATTTCTTTTCCAC | |
| | TTTCCCAATTGGATGCATTAGCTTCAACCTGACAACAGGTGT | |
| | GTCTGGAGCCTTCTGGACCATCGCCTATACCTCTTACACAGA | |
| | GGCTCTAGTACAGGTGGAGAACACAGCTATAAAGAACGTGA | |
| | CGTACTGTAACAGTCACATAAACAATATCAAGTGTTCTCAGTT | |
| | GACTGCGAACTTAAACAATGGGTTTTATCCAGTGGCGAGCTC | |
| | GGAGGTGGGGTTTGTAAACAAATCTGTGGTGCTGTTGCCCTC | |
| | CTTCTTCACGTACACTGCAGTGAACATCACCATTGATTTGGG | |
| | GATGAAACTGTCCGGCTACGGGCAGCCTATAGCATCTACACT | |
| | GAGCAATATCACACTGCCCATGCAGGATAACAATACAGATGT | |
| | GTACTGTATCCGCTCAAACCAGTTCTCTGTATACGTGCACAGT | |
| | ACATGCAAGAGCTCGCTATGGGACAACATTTTCAACCAGGAT | |
| | TGTACTGATGTGCTTGAAGCAACTGCAGTGATCAAAACAGGC | |
| | ACATGCCCGTTCAGCTTTGATAAGCTCAACAACTACCTAACG | |
| | TTCAACAAGTTCTGCTTGAGCCTGTCTCCAGTAGGCGCCAAT | |
| | TGCAAGTTTGACGTTGCAGCGCGAACACGGACAAACGAACA | |
| | GGTAGTGCGGTCGCTCTATGTTATCTACGAGGAGGGGGACAA | |
| | CATAGTCGGGGTTCCATCCGACAACTCAGGTTTGCACGACCT | |
| | GAGTGTGCTCCATTTGGACTCATGCACGGATTATAACATCTAC | |
| | GGGCGCACAGGTGTGGGGATAATACGAAGAACAAACTCTAC | |
| | GCTATTGAGCGGGCTCTACTACACCTCATTGAGTGGGGACCT | |
| | GCTAGGGTTCAAGAACGTATCTGACGGTGTGATCTATAGCGT | |
| | CACACCATGTGACGTATCAGCCCAAGCTGCTGTGATTGACGG | |
| | GGCGATTGTGGGGGCTATGACTTCAATTAACAGCGAGCTCCT | |
| | AGGCCTGACCCACTGGACTACCACCCCAAACTTCTACTACTA | |
| | CAGCATTTATAACTATACCAGTGAGCGCACCAGGGACACTGC | |
| | CATTGACAGCAATGACGTCGACTGCGAGCCTGTTATTACCTA | |
| | CAGCAACATCGGTGTTTGTAAGAATGGAGCTCTAGTCTTCAT | |
| | AAACGTAACGCACTCTGATGGCGATGTTCAACCAATTTCCAC | |
| | TGGGAACGTAACCATACCCACCAACTTTACTATTTCCGTCCAG | |
| | GTGGAGTACATGCAAGTATATACCACGCCAGTGTCCATCGAC | |
| | TGCGCTCGGTATGTGTGCAACGGTAACCCACGCTGCAATAAG | |
| | CTGCTAACGCAGTACGTCAGCGCCTGCCAGACAATAGAGCA | |
| | GGCATTGGCAATGGGTGCAAGGCTTGAAAACATGGAGGTGG | |
| | ACTCCATGTTGTTCGTGTCTGAAAACGCTCTTAAACTAGCATC | |
| | CGTGGAGGCATTCAACAGTACTGAGAACTTGGACTCTATCTA | |
| | TAAGGAGTGGCCCTCCATTGGGGGCAGCTGGCTTGGAGGTCT | |
| | AAAAGACATCCTGCCCAGCCACAACTCCAAGAGGAAGTACG | |
| | GGTCCGCTATAGAGGACCTCCTCTTTGACAAGGTTGTTACTT | |
| | CTGGTCTTGGCACAGTGGACGAAGACTACAAGAGGTGCACA | |
| | GGAGGCTATGATATAGCTGACCTGGTGTGTGCTCAATACTACA | |
| | ACGGTATAATGGTTCTCCCAGGTGTGGCCAACGCTGACAAGA | |
| | TGACAATGTACACAGCCTCTTTAGCTGGAGGCATTACCCTGG | |
| | GAGCCCTTGGGGGTGGCGCAGTGGCAATTCCATTTGCCGTTG | |
| | CGGTGCAGGCCCGACTAAACTATGTCGCACTTCAAACAGATG | |
| | TGCTCAACAAGAACCAACAAATACTGGCCAACGCTTTCAAC | |
| | CAGGCCATTGGTAACATTACGCAGGCATTTGGCAAGGTGAAT | |
| | GACGCCATCCACCAGACCAGCCAGGGACTTGCCACAGTGGC | |
| | CAAGGCCTTGGCAAAGGTGCAGGATGTCGTGAACACACAGG | |
| | GTCAGGCCCTCTCTCATTTGACAGTGCAGCTTCAGAATAACT | |
| | TCCAAGCAATCAGTTCAAGCATCAGCGACATCTACAACCGGC | |
| | TGGACCCCCCATCTGCAGATGCGCAGGTGGACAGGCTAATCA | |
| | CTGGACGCTTGACGGCACTAAATGCCTTTGTCAGCCAAACTC | |
| | TGACCCGGCAAGCAGAGGTGCGGGCCAGTAGACAACTGGCC | |
| | AAAGACAAGGTCAACGAGTGCGTCAGGTCCCAGTCCCAGCG | |
| | TTTTGGATTCTGTGGGAACGGGACGCACCTGTTCTCATTAGC | |
| | CAATGCTGCACCCAATGGCATGATCTTTTTCCATACTGTTCTA | |
| | CTTCCTACTGCCTATGAAACCGTGACCGCTTGGAGCGGCATC | |
| | TGCGCATCTGATGGCGATAGGACCTTCGGGCTGGTCGTTAAG | |
| | GATGTCCAGCTAACGCTGTTCCGGAACTTGGATGACAAGTTC | |
| | TACCTGACCCCCAGGACCATGTACCAGCCGAGAGTGGCAAC | |
| | GAGTTCTGACTTCGTGCAAATTGAGGGCTGTGACGTCCTGTT | |
| | TGTTAATGCAACAGTGATCGATCTGCCCAGTATCATACCAGAT | |
| | TACATAGACATAAACCAGACAGTCCAGGACATACTGGAGAAT | |
| | TACAGGCCAAACTGGACCGTACCAGAGTTCACGCTGGACAT | |
| | ATTCAACGCTACGTACCTCAATTTGACTGGGGAAATTGATGA | |
| | CTTGGAGTTCAGGTCGGAGAAGCTCCACAACACCACTGTGG | |
| | AGCTGGCCATCCTGATTGACAACATCAACAACACTCTGGTGA | |
| | ACCTGGAGTGGCTAAATCGCATTGAAACCTATGTCAAGTGGC | |
| | CTTGGTACGTTTGGCTACTGATCGGACTCGTGGTAGTCTTCTG | |
| | CATACCACTCCTGCTATTTTGCTGCTTCAGCACAGGGTGCTGT | |
| | GGCTGCATTGGATGCCTAGGTTCCTGCTGTCACAGTATCTGCA | |
| | GCAGAAGACAATTCGAGAACTACGAGCCCATAGAAAAGGTC | |
| | CACGTACAT | |
| 172 | ATGAGATTTGTGATGTCCCCCACTGTCCTGCTGCTGCTCCTGG | MHCIsp- |
| | GAGCCCTGGCAGCCCCCCAGACCTGGGCAGGCAGCACCACC | S_ec(S2) |
| | AACAATGAATGCATCCAGGTGAACGTGACCCAGCTGGCAGG | |
| | CAATGAAAATTTGATCAGAGACTTCCTGTTCAGCAACTTCAA | |
| | GGAGGAGGGCAGTGTAGTGGTGGGAGGCTACTACCCAACAG | |
| | AGGTGTGGTACAACTGCAGCAGAACAGCCAGAACCACAGCC | |
| | TTCCAGTACTTCAACAACATCCACGCCTTCTACTTTGTGATGG | |
| | AGGCCATGGAAAACAGCACAGGAAATGCCAGAGGAAAACC | |
| | CCTGCTCTTCCACGTGCACGGAGAGCCCGTGTCAGTCATCAT | |
| | CAGCGCCTACAGAGATGACGTCCAGCAGCGGCCCCTGCTGA | |
| | AGCATGGACTGGTCTGCATCACCAAGAACAGACACATCAAC | |
| | TACGAGCAGTTCACCAGCAACCAGTGGAACAGCACCTGCAC | |
| | AGGAGCAGACAGAAAAATCCCCTTCAGCGTCATCCCCACAG | |
| | ACAACGGCACCAAAATCTATGGCCTGGAGTGGAATGATGACT | |
| | TTGTGACAGCCTATATCAGCGGCAGGAGCTACCACCTCAACA | |
| | TCAACACCAACTGGTTCAACAACGTCACCCTGCTCTACTCCA | |
| | GATCCAGCACAGCCACCTGGGAGTACAGCGCCGCCTATGCCT | |
| | ACCAGGGAGTCTCCAACTTCACCTACTACAAACTGAACAAC | |
| | ACCAACGGCCTGAAAACCTACGAGCTGTGTGAGGACTACGA | |
| | GCACTGCACAGGCTATGCCACAAATGTGTTTGCCCCAACCAG | |
| | CGGAGGCTACATCCCAGACGGCTTCTCCTTCAACAACTGGTT | |
| | CCTCCTCACCAACTCCTCCACATTTGTGAGCGGCAGATTTGT | |
| | GACCAACCAGCCCCTGCTGATCAACTGCCTGTGGCCCGTGCC | |
| | CAGCTTTGGAGTGGCAGCCCAGGAGTTCTGCTTCGAGGGAG | |
| | CCCAGTTCAGCCAGTGCAACGGAGTCAGCCTGAACAACACA | |
| | GTGGACGTGATCAGATTCAACCTGAACTTCACAGCAGACGT | |
| | GCAGAGTGGAATGGGAGCCACCGTCTTCAGCCTGAACACCA | |
| | CAGGAGGAGTGATCCTGGAGATCAGCTGCTACAGCGACACA | |
| | GTGAGCGAGAGCAGCAGCTACAGCTACGGAGAGATCCCATT | |
| | TGGCATCACAGATGGCCCCAGGTACTGCTACGTCCTGTACAA | |
| | TGGAACAGCCCTGAAATACCTGGGCACCCTCCCACCCAGCG | |
| | TGAAGGAGATCGCCATCAGCAAGTGGGGCCACTTCTACATCA | |
| | ATGGCTACAACTTCTTCAGCACCTTCCCCATCGGCTGCATCTC | |
| | CTTCAACCTGACCACAGGAGTGAGCGGGGCCTTCTGGACAA | |
| | TCGCCTACACATCCTACACAGAAGCCCTGGTGCAGGTGGAG | |
| | AACACAGCCATCAAAAACGTCACCTACTGCAACAGCCACAT | |
| | CAACAACATCAAGTGCAGCCAGCTGACAGCCAACCTGAACA | |
| | ACGGCTTCTACCCAGTGGCCAGCTCAGAGGTGGGCTTCGTG | |
| | AACAAGAGCGTGGTGCTCCTGCCCAGCTTCTTCACCTACACA | |
| | GCAGTGAACATCACAATTGACCTGGGCATGAAGCTGAGCGG | |
| | CTACGGCCAGCCAATTGCCAGCACCCTCTCCAACATCACCCT | |
| | CCCCATGCAGGACAATAACACAGATGTGTACTGCATCAGATC | |
| | CAACCAGTTCTCTGTCTACGTGCACAGCACCTGCAAAAGCA | |
| | GCCTGTGGGACAACATCTTCAACCAGGACTGCACAGATGTCC | |
| | TGGAGGCCACAGCCGTGATCAAAACAGGCACCTGCCCCTTC | |
| | AGCTTTGACAAACTCAACAACTACCTTACATTCAACAAATTC | |
| | TGCCTCTCCCTCAGCCCAGTGGGAGCCAACTGCAAGTTTGAT | |
| | GTGGCCGCCAGGACCAGGACAAATGAACAAGTGGTCAGAA | |
| | GCCTCTACGTCATCTACGAGGAGGGAGACAACATCGTGGGG | |
| | GTCCCCAGCGACAACAGCGGCCTGCACGACCTGAGTGTGCT | |
| | CCACCTGGACAGCTGCACAGACTACAACATCTACGGCAGGA | |
| | CTGGGGTGGGCATCATCAGAAGAACCAACAGCACACTGCTG | |
| | AGTGGCCTGTACTACACCAGCCTGAGTGGAGACTTGCTGGG | |
| | CTTCAAGAATGTGTCAGATGGGGTGATCTACAGTGTGACCCC | |
| | CTGTGACGTGTCTGCCCAGGCTGCAGTCATCGACGGAGCCAT | |
| | CGTGGGAGCCATGACCAGCATTAACAGCGAGCTGCTGGGCC | |
| | TGACCCACTGGACCACCACCCCCAACTTCTACTACTACTCCA | |
| | TCTACAACTACACATCAGAAAGAACAAGAGACACAGCCATC | |
| | GACAGCAATGACGTGGACTGTGAGCCAGTCATCACCTACAG | |
| | CAACATCGGAGTGTGCAAGAACGGAGCCCTGGTGTTCATCA | |
| | ACGTGACCCACAGCGACGGAGATGTCCAGCCCATCAGCACA | |
| | GGAAATGTGACCATCCCAACCAACTTCACCATCAGCGTCCAG | |
| | GTGGAATACATGCAGGTGTACACCACCCCAGTGTCCATCGAC | |
| | TGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCC | |
| 173 | ATGAGATTTGTGATGAGCCCCACTGTGCTGCTGCTGCTGCTG | MHCIsp- |
| | GGAGCCCTGGCAGCCCCCCAGACCTGGGCTGGCTCAACCAC | S_ec(S3) |
| | CAACAACGAGTGCATCCAGGTGAACGTGACCCAGCTGGCAG | |
| | GCAACGAGAACCTCATCAGAGACTTCCTCTTCTCCAACTTCA | |
| | AGGAGGAGGGCTCAGTGGTGGTCGGCGGCTACTACCCAACA | |
| | GAGGTGTGGTACAACTGCTCAAGGACCGCCAGAACCACAGC | |
| | CTTCCAGTACTTCAACAACATCCACGCCTTCTACTTCGTGATG | |
| | GAGGCCATGGAGAACTCCACCGGGAACGCCAGGGGCAAGC | |
| | CACTACTCTTCCACGTGCACGGAGAGCCAGTGAGCGTGATCA | |
| | TCTCAGCCTACAGGGACGACGTGCAGCAGCGCCCCCTGCTG | |
| | AAGCATGGACTGGTGTGCATCACCAAGAACAGGCACATCAA | |
| | CTACGAGCAGTTCACCAGCAACCAGTGGAACAGCACCTGCA | |
| | CCGGCGCAGACAGGAAGATCCCCTTCTCAGTGATCCCAACA | |
| | GACAACGGAACCAAAATCTACGGCCTGGAGTGGAACGACGA | |
| | CTTCGTGACCGCCTACATCAGCGGCAGGTCCTACCATCTCAA | |
| | CATCAACACCAACTGGTTCAACAACGTCACCCTCCTCTACAG | |
| | CAGGTCATCCACAGCCACCTGGGAGTACTCAGCTGCCTATGC | |
| | ATACCAGGGAGTCTCCAACTTCACATACTACAAACTCAACAA | |
| | CACCAACGGCCTCAAGACCTACGAGCTGTGTGAGGACTACG | |
| | AGCACTGCACCGGCTACGCAACAAACGTCTTCGCCCCAACC | |
| | TCCGGAGGCTACATCCCAGACGGCTTCTCCTTCAACAACTGG | |
| | TTCCTCCTCACAAACAGCTCCACCTTCGTGTCAGGAAGGTTC | |
| | GTGACCAACCAGCCCCTGCTCATCAACTGCCTCTGGCCCGTC | |
| | CCCTCCTTCGGAGTGGCCGCCCAGGAGTTCTGCTTCGAGGG | |
| | AGCCCAGTTCTCCCAGTGCAACGGAGTCTCCCTCAACAACA | |
| | CCGTGGACGTCATCAGATTCAACCTCAACTTCACAGCAGACG | |
| | TCCAGAGCGGCATGGGAGCCACCGTGTTCAGCCTGAACACC | |
| | ACAGGAGGAGTGATCCTGGAGATCTCCTGCTACTCAGACACA | |
| | GTGTCAGAGTCCTCCTCCTACAGCTACGGAGAGATCCCATTC | |
| | GGCATCACAGACGGCCCCAGATACTGCTACGTGCTGTACAAC | |
| | GGCACAGCCCTGAAGTACCTGGGCACCCTCCCCCCATCAGTG | |
| | AAGGAGATCGCCATCAGCAAGTGGGGCCACTTCTACATCAAC | |
| | GGCTACAACTTCTTCTCCACCTTCCCCATCGGCTGCATCAGCT | |
| | TCAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCC | |
| 174 | ATGCGTTTTGTAATGTCACCTACTGTACTACTACTACTACTCG | MHCIsp- |
| | GAGCACTAGCAGCACCTCAGACTTGGGCCGGATCAACCACA | S_ec(S6) |
| | AATAACGAGTGCATTCAGGTCAACGTCACCCAGCTGGCCGGT | |
| | AACGAGAACCTAATTAGAGACTTCCTATTCTCGAACTTTAAA | |
| | GAGGAAGGCTCTGTGGTGGTCGGAGGTTACTACCCCACAGA | |
| | AGTGTGGTACAATTGCTCACGTACAGCCAGGACCACTGCCTT | |
| | CCAGTACTTCAACAACATTCATGCCTTCTACTTTGTCATGGAA | |
| | GCCATGGAGAACTCCACTGGGAATGCCAGAGGAAAGCCTCT | |
| | CCTCTTCCATGTCCATGGAGAGCCTGTCTCTGTGATTATCTCA | |
| | GCATATAGGGATGATGTGCAGCAGCGGCCGCTGCTTAAGCAT | |
| | GGCCTAGTGTGCATTACTAAGAACCGACATATCAATTATGAGC | |
| | AGTTCACCTCCAACCAGTGGAACTCCACATGCACTGGTGCTG | |
| | ATAGGAAGATCCCGTTCAGCGTTATCCCCACCGATAATGGCA | |
| | CAAAGATTTATGGCCTAGAATGGAACGATGATTTTGTTACTGC | |
| | CTACATATCAGGAAGAAGTTACCACTTAAACATTAACACCAA | |
| | TTGGTTCAATAATGTTACACTTCTGTACTCTCGCAGCAGTACG | |
| | GCCACTTGGGAGTATTCGGCTGCATATGCCTACCAAGGTGTA | |
| | AGCAACTTCACCTACTACAAGCTGAACAATACGAACGGTCTG | |
| | AAGACTTATGAGCTGTGCGAAGACTACGAGCACTGTACGGG | |
| | CTATGCGACAAATGTCTTCGCCCCGACGAGCGGCGGGTACAT | |
| | ACCGGATGGCTTCTCCTTCAACAACTGGTTCCTCCTTACCAAT | |
| | AGCTCCACTTTCGTATCAGGAAGATTTGTTACGAACCAACCC | |
| | CTTCTCATTAACTGTCTGTGGCCAGTGCCCTCCTTCGGAGTAG | |
| | CTGCTCAAGAGTTCTGTTTCGAGGGTGCACAGTTCAGCCAGT | |
| | GTAATGGAGTGTCGCTGAACAACACTGTGGACGTGATCAGGT | |
| | TTAATTTGAACTTCACAGCTGATGTTCAGTCCGGCATGGGCG | |
| | CGACTGTGTTCAGCCTAAACACCACGGGTGGCGTCATCTTGG | |
| | AGATTAGTTGTTACTCTGACACTGTGTCAGAGAGCAGCAGTT | |
| | ACTCCTACGGAGAAATTCCTTTCGGCATCACAGACGGTCCCC | |
| | GGTACTGCTATGTGCTGTACAACGGAACTGCTTTGAAGTACC | |
| | TGGGGACATTGCCACCTTCTGTGAAGGAAATAGCCATCTCTA | |
| | AGTGGGGTCACTTTTACATTAACGGCTATAATTTCTTTTCCAC | |
| | TTTCCCAATTGGATGCATTAGCTTCAACCTGACAACAGGTGT | |
| | GTCTGGAGCCTTCTGGACCATCGCCTATACCTCTTACACAGA | |
| | GGCTCTAGTACAGGTGGAGAACACAGCTATAAAGAACGTGA | |
| | CGTACTGTAACAGTCACATAAACAATATCAAGTGTTCTCAGTT | |
| | GACTGCGAACTTAAACAATGGGTTTTATCCAGTGGCGAGCTC | |
| | GGAGGTGGGGTTTGTAAACAAATCTGTGGTGCTGTTGCCCTC | |
| | CTTCTTCACGTACACTGCAGTGAACATCACCATTGATTTGGG | |
| | GATGAAACTGTCCGGCTACGGGCAGCCTATAGCATCTACACT | |
| | GAGCAATATCACACTGCCCATGCAGGATAACAATACAGATGT | |
| | GTACTGTATCCGCTCAAACCAGTTCTCTGTATACGTGCACAGT | |
| | ACATGCAAGAGCTCGCTATGGGACAACATTTTCAACCAGGAT | |
| | TGTACTGATGTGCTTGAAGCAACTGCAGTGATCAAAACAGGC | |
| | ACATGCCCGTTCAGCTTTGATAAGCTCAACAACTACCTAACG | |
| | TTCAACAAGTTCTGCTTGAGCCTGTCTCCAGTAGGCGCCAAT | |
| | TGCAAGTTTGACGTTGCAGCGCGAACACGGACAAACGAACA | |
| | GGTAGTGCGGTCGCTCTATGTTATCTACGAGGAGGGGGACAA | |
| | CATAGTCGGGGTTCCATCCGACAACTCAGGTTTGCACGACCT | |
| | GAGTGTGCTCCATTTGGACTCATGCACGGATTATAACATCTAC | |
| | GGGCGCACAGGTGTGGGGATAATACGAAGAACAAACTCTAC | |
| | GCTATTGAGCGGGCTCTACTACACCTCATTGAGTGGGGACCT | |
| | GCTAGGGTTCAAGAACGTATCTGACGGTGTGATCTATAGCGT | |
| | CACACCATGTGACGTATCAGCCCAAGCTGCTGTGATTGACGG | |
| | GGCGATTGTGGGGGCTATGACTTCAATTAACAGCGAGCTCCT | |
| | AGGCCTGACCCACTGGACTACCACCCCAAACTTCTACTACTA | |
| | CAGCATTTATAACTATACCAGTGAGCGCACCAGGGACACTGC | |
| | CATTGACAGCAATGACGTCGACTGCGAGCCTGTTATTACCTA | |
| | CAGCAACATCGGTGTTTGTAAGAATGGAGCTCTAGTCTTCAT | |
| | AAACGTAACGCACTCTGATGGCGATGTTCAACCAATTTCCAC | |
| | TGGGAACGTAACCATACCCACCAACTTTACTATTTCCGTCCAG | |
| | GTGGAGTACATGCAAGTATATACCACGCCAGTGTCCATCGAC | |
| | TGCGCTCGGTATGTGTGCAACGGTAACCCACGCTGCAATAAG | |
| | CTGCTAACGCAGTACGTCAGCGCCTGCCAGACAATAGAGCA | |
| | GGCATTGGCAATGGGTGCAAGGCTTGAAAACATGGAGGTGG | |
| | ACTCCATGTTGTTCGTGTCTGAAAACGCTCTTAAACTAGCATC | |
| | CGTGGAGGCATTCAACAGTACTGAGAACTTGGACTCTATCTA | |
| | TAAGGAGTGGCCCTCCATTGGGGGCAGCTGGCTTGGAGGTCT | |
| | AAAAGACATCCTGCCCAGCCACAACTCCAAGAGGAAGTACG | |
| | GGTCCGCTATAGAGGACCTCCTCTTTGACAAGGTTGTTACTT | |
| | CTGGTCTTGGCACAGTGGACGAAGACTACAAGAGGTGCACA | |
| | GGAGGCTATGATATAGCTGACCTGGTGTGTGCTCAATACTACA | |
| | ACGGTATAATGGTTCTCCCAGGTGTGGCCAACGCTGACAAGA | |
| | TGACAATGTACACAGCCTCTTTAGCTGGAGGCATTACCCTGG | |
| | GAGCCCTTGGGGGTGGCGCAGTGGCAATTCCATTTGCCGTTG | |
| | CGGTGCAGGCCCGACTAAACTATGTCGCACTTCAAACAGATG | |
| | TGCTCAACAAGAACCAACAAATACTGGCCAACGCTTTCAAC | |
| | CAGGCCATTGGTAACATTACGCAGGCATTTGGCAAGGTGAAT | |
| | GACGCCATCCACCAGACCAGCCAGGGACTTGCCACAGTGGC | |
| | CAAGGCCTTGGCAAAGGTGCAGGATGTCGTGAACACACAGG | |
| | GTCAGGCCCTCTCTCATTTGACAGTGCAGCTTCAGAATAACT | |
| | TCCAAGCAATCAGTTCAAGCATCAGCGACATCTACAACCGGC | |
| | TGGACCCCCCATCTGCAGATGCGCAGGTGGACAGGCTAATCA | |
| | CTGGACGCTTGACGGCACTAAATGCCTTTGTCAGCCAAACTC | |
| | TGACCCGGCAAGCAGAGGTGCGGGCCAGTAGACAACTGGCC | |
| | AAAGACAAGGTCAACGAGTGCGTCAGGTCCCAGTCCCAGCG | |
| | TTTTGGATTCTGTGGGAACGGGACGCACCTGTTCTCATTAGC | |
| | CAATGCTGCACCCAATGGCATGATCTTTTTCCATACTGTTCTA | |
| | CTTCCTACTGCCTATGAAACCGTGACCGCTTGGAGCGGCATC | |
| | TGCGCATCTGATGGCGATAGGACCTTCGGGCTGGTCGTTAAG | |
| | GATGTCCAGCTAACGCTGTTCCGGAACTTGGATGACAAGTTC | |
| | TACCTGACCCCCAGGACCATGTACCAGCCGAGAGTGGCAAC | |
| | GAGTTCTGACTTCGTGCAAATTGAGGGCTGTGACGTCCTGTT | |
| | TGTTAATGCAACAGTGATCGATCTGCCCAGTATCATACCAGAT | |
| | TACATAGACATAAACCAGACAGTCCAGGACATACTGGAGAAT | |
| | TACAGGCCAAACTGGACCGTACCAGAGTTCACGCTGGACAT | |
| | ATTCAACGCTACGTACCTCAATTTGACTGGGGAAATTGATGA | |
| | CTTGGAGTTCAGGTCGGAGAAGCTCCACAACACCACTGTGG | |
| | AGCTGGCCATCCTGATTGACAACATCAACAACACTCTGGTGA | |
| | ACCTGGAGTGGCTAAATCGCATTGAAACCTATGTCAAGTGGC | |
| | CT | |
| 175 | ATGAGATTTGTGATGTCCCCCACTGTCCTGCTGCTGCTCCTGG | MHCIsp- |
| | GAGCCCTGGCAGCCCCCCAGACCTGGGCAGGCAGCTGTGAG | SII(S2) |
| | CCAGTCATCACCTACAGCAACATCGGAGTGTGCAAGAACGG | |
| | AGCCCTGGTGTTCATCAACGTGACCCACAGCGACGGAGATG | |
| | TCCAGCCCATCAGCACAGGAAATGTGACCATCCCAACCAACT | |
| | TCACCATCAGCGTCCAGGTGGAATACATGCAGGTGTACACCA | |
| | CCCCAGTGTCCATCGACTGTGCCAGATACGTGTGCAATGGAA | |
| | ACCCCAGATGCAACAAGCTCCTCACCCAGTACGTGTCAGCCT | |
| | GCCAGACAATCGAGCAGGCCCTGGCCATGGGAGCCAGGCTC | |
| | GAGAACATGGAAGTGGACAGCATGCTGTTTGTCTCAGAGAA | |
| | TGCCCTGAAACTGGCCAGCGTGGAGGCCTTCAACAGCACAG | |
| | AGAACCTGGACAGCATCTACAAGGAGTGGCCATCAATCGGA | |
| | GGCAGCTGGCTGGGAGGACTTAAGGACATCCTGCCAAGCCA | |
| | CAACAGCAAAAGAAAGTACGGCAGCGCCATTGAGGACCTGC | |
| | TGTTTGACAAGGTGGTCACCTCCGGCCTGGGCACAGTGGAT | |
| | GAGGACTACAAGAGATGCACCGGCGGCTATGACATTGCCGA | |
| | CCTGGTGTGTGCCCAGTACTACAATGGCATCATGGTGCTGCC | |
| | TGGAGTGGCCAACGCCGACAAAATGACCATGTACACCGCCT | |
| | CCCTGGCTGGAGGCATCACACTGGGAGCCCTGGGGGGAGGA | |
| | GCAGTGGCCATCCCCTTTGCAGTGGCTGTGCAGGCCAGACTC | |
| | AACTACGTGGCCCTGCAGACAGACGTGCTCAACAAGAACCA | |
| | GCAGATCCTGGCCAACGCTTTCAACCAGGCTATCGGAAACAT | |
| | CACCCAGGCCTTTGGAAAAGTGAATGATGCCATCCACCAGAC | |
| | CAGCCAGGGCCTGGCCACAGTGGCCAAGGCCCTGGCCAAGG | |
| | TGCAGGACGTGGTCAACACCCAGGGCCAGGCCCTCAGTCAC | |
| | CTCACAGTACAGCTCCAGAACAACTTCCAGGCAATCTCCTCC | |
| | TCCATCAGCGACATCTACAACAGGCTGGACCCCCCAAGCGCT | |
| | GATGCCCAGGTGGACAGACTGATCACAGGAAGACTCACAGC | |
| | CCTCAACGCATTTGTGTCCCAGACACTGACCAGGCAGGCAG | |
| | AGGTCAGGGCCAGCAGGCAGCTGGCCAAGGACAAGGTGAA | |
| | TGAGTGCGTGAGGAGCCAGAGCCAGAGATTTGGCTTCTGCG | |
| | GAAACGGCACCCACCTGTTCAGCCTGGCCAACGCCGCCCCC | |
| | AACGGCATGATTTTCTTCCACACAGTCCTCCTCCCCACAGCC | |
| | TACGAAACAGTGACAGCCTGGTCAGGCATCTGTGCCAGCGA | |
| | CGGAGACAGAACCTTTGGCCTGGTGGTGAAGGATGTGCAGC | |
| | TCACCCTCTTCAGAAACCTGGATGACAAGTTCTACCTCACCC | |
| | CAAGAACCATGTACCAGCCCAGAGTGGCCACAAGCAGCGAC | |
| | TTTGTGCAGATTGAGGGCTGTGACGTGCTGTTTGTGAATGCA | |
| | ACAGTGATTGACCTCCCAAGCATCATCCCAGATTACATCGAC | |
| | ATCAACCAGACAGTGCAGGACATCCTGGAGAACTACAGGCC | |
| | CAACTGGACAGTGCCAGAGTTCACCCTGGACATCTTCAACG | |
| | CCACCTACCTGAACCTGACAGGAGAAATTGACGACCTGGAG | |
| | TTCAGATCAGAAAAACTTCACAACACCACCGTGGAGCTTGC | |
| | CATCCTCATTGACAACATTAACAACACACTGGTCAACCTGGA | |
| | ATGGCTGAACAGAATTGAAACCTACGTGAAGTGGCCCTGGTA | |
| | TGTGTGGCTGCTGATTGGACTGGTGGTGGTGTTCTGCATCCC | |
| | ACTGCTGCTGTTCTGCTGCTTCAGCACCGGCTGCTGTGGATG | |
| | CATCGGCTGCTTGGGCAGCTGCTGCCACAGCATCTGCAGCAG | |
| | GAGGCAGTTTGAGAACTACGAACCAATTGAAAAAGTGCACG | |
| | TCCAC | |
| 176 | ATGAGATTTGTGATGAGCCCCACTGTGCTGCTGCTGCTGCTG | MHCIsp- |
| | GGAGCCCTGGCAGCCCCCCAGACCTGGGCTGGCTCATGTGA | SII(S3) |
| | GCCAGTCATCACCTACTCCAACATCGGCGTGTGCAAGAACGG | |
| | AGCCCTGGTGTTCATCAACGTCACCCACTCAGACGGCGACGT | |
| | CCAGCCAATCTCCACAGGAAACGTCACCATCCCCACCAACTT | |
| | CACCATCAGCGTGCAGGTGGAGTACATGCAGGTCTACACCAC | |
| | CCCAGTCTCCATCGACTGTGCCAGGTACGTGTGCAACGGCAA | |
| | CCCAAGATGCAACAAACTGCTGACCCAGTACGTGAGCGCCT | |
| | GCCAGACCATCGAGCAGGCCCTGGCCATGGGCGCCAGGCTG | |
| | GAGAACATGGAGGTGGACAGCATGCTCTTTGTGAGCGAGAA | |
| | CGCCCTGAAGCTTGCCAGCGTGGAGGCCTTCAACAGCACCG | |
| | AAAACCTGGACTCCATCTACAAAGAGTGGCCCTCCATAGGAG | |
| | GCTCCTGGCTGGGAGGCCTGAAGGACATCCTCCCATCCCACA | |
| | ACAGCAAAAGAAAGTACGGCAGCGCCATCGAAGACCTGCTG | |
| | TTCGACAAGGTGGTCACCTCAGGACTGGGCACAGTGGACGA | |
| | GGACTACAAGAGGTGCACCGGAGGCTACGACATCGCAGACC | |
| | TGGTCTGTGCCCAGTACTACAACGGCATCATGGTGCTCCCAG | |
| | GCGTGGCCAACGCCGACAAGATGACCATGTACACAGCAAGC | |
| | CTGGCTGGAGGAATCACACTGGGAGCCCTGGGAGGAGGGGC | |
| | CGTGGCCATTCCATTCGCCGTGGCCGTGCAGGCCAGACTGAA | |
| | CTACGTGGCCCTGCAGACAGACGTGCTAAACAAGAACCAGC | |
| | AGATCCTGGCCAACGCCTTCAACCAGGCCATCGGCAACATCA | |
| | CCCAGGCCTTCGGCAAGGTGAACGACGCAATCCACCAGACA | |
| | TCACAGGGCCTGGCAACAGTGGCCAAGGCCCTGGCCAAGGT | |
| | CCAGGACGTGGTGAACACCCAGGGCCAGGCCCTCTCACACC | |
| | TGACAGTCCAGCTGCAGAACAACTTCCAGGCAATCTCCTCCT | |
| | CCATCTCAGACATCTACAACAGACTGGACCCCCCCTCAGCCG | |
| | ACGCCCAGGTGGACAGACTCATCACAGGCAGGCTGACCGCC | |
| | CTCAACGCCTTCGTGTCCCAGACCCTCACCAGGCAGGCCGA | |
| | GGTGAGGGCCAGCAGGCAGCTCGCCAAGGACAAGGTGAAC | |
| | GAGTGCGTCAGAAGCCAGAGCCAGAGGTTCGGCTTCTGTGG | |
| | CAACGGCACCCACCTGTTCTCCCTGGCCAACGCAGCCCCCA | |
| | ACGGCATGATCTTCTTCCACACAGTCCTCCTCCCAACAGCAT | |
| | ATGAGACAGTCACCGCCTGGTCAGGAATCTGTGCCTCAGAC | |
| | GGGGACAGAACCTTCGGCCTGGTGGTCAAGGACGTGCAGCT | |
| | GACACTCTTCAGAAACCTGGACGACAAATTCTACCTGACCCC | |
| | CAGGACCATGTACCAGCCAAGGGTGGCCACCTCCTCAGACTT | |
| | CGTGCAGATCGAGGGCTGTGACGTGCTCTTCGTGAACGCCA | |
| | CCGTCATCGACCTCCCATCCATCATCCCAGACTACATCGACAT | |
| | CAACCAGACAGTGCAGGACATCCTGGAGAACTACCGCCCCA | |
| | ACTGGACCGTGCCAGAGTTCACCCTAGACATATTCAACGCCA | |
| | CCTACCTGAACCTGACAGGAGAAATTGACGACCTGGAGTTC | |
| | AGATCAGAAAAGCTACACAACACCACCGTGGAGTTAGCCAT | |
| | CCTCATAGACAACATTAACAACACCCTCGTCAACCTGGAGTG | |
| | GCTCAACAGGATTGAAACCTACGTGAAGTGGCCCTGGTACGT | |
| | CTGGCTCCTCATCGGCCTGGTGGTGGTCTTCTGCATCCCACT | |
| | GCTGCTGTTCTGCTGCTTCTCCACCGGCTGCTGTGGATGCATC | |
| | GGCTGCCTGGGCTCATGCTGCCACTCAATCTGCTCAAGGAGG | |
| | CAGTTTGAAAACTACGAGCCAATAGAAAAAGTCCACGTCCA | |
| | C | |
| 177 | ATGCGTTTTGTAATGTCACCTACTGTACTACTACTACTACTCG | MHCIsp- |
| | GAGCACTAGCAGCACCTCAGACTTGGGCCGGATCATGCGAG | SII(S6) |
| | CCTGTTATTACCTACAGCAACATCGGTGTTTGTAAGAATGGAG | |
| | CTCTAGTCTTCATAAACGTAACGCACTCTGATGGCGATGTTCA | |
| | ACCAATTTCCACTGGGAACGTAACCATACCCACCAACTTTAC | |
| | TATTTCCGTCCAGGTGGAGTACATGCAAGTATATACCACGCCA | |
| | GTGTCCATCGACTGCGCTCGGTATGTGTGCAACGGTAACCCA | |
| | CGCTGCAATAAGCTGCTAACGCAGTACGTCAGCGCCTGCCAG | |
| | ACAATAGAGCAGGCATTGGCAATGGGTGCAAGGCTTGAAAA | |
| | CATGGAGGTGGACTCCATGTTGTTCGTGTCTGAAAACGCTCT | |
| | TAAACTAGCATCCGTGGAGGCATTCAACAGTACTGAGAACTT | |
| | GGACTCTATCTATAAGGAGTGGCCCTCCATTGGGGGCAGCTG | |
| | GCTTGGAGGTCTAAAAGACATCCTGCCCAGCCACAACTCCA | |
| | AGAGGAAGTACGGGTCCGCTATAGAGGACCTCCTCTTTGACA | |
| | AGGTTGTTACTTCTGGTCTTGGCACAGTGGACGAAGACTACA | |
| | AGAGGTGCACAGGAGGCTATGATATAGCTGACCTGGTGTGTG | |
| | CTCAATACTACAACGGTATAATGGTTCTCCCAGGTGTGGCCA | |
| | ACGCTGACAAGATGACAATGTACACAGCCTCTTTAGCTGGAG | |
| | GCATTACCCTGGGAGCCCTTGGGGGTGGCGCAGTGGCAATTC | |
| | CATTTGCCGTTGCGGTGCAGGCCCGACTAAACTATGTCGCAC | |
| | TTCAAACAGATGTGCTCAACAAGAACCAACAAATACTGGCC | |
| | AACGCTTTCAACCAGGCCATTGGTAACATTACGCAGGCATTT | |
| | GGCAAGGTGAATGACGCCATCCACCAGACCAGCCAGGGACT | |
| | TGCCACAGTGGCCAAGGCCTTGGCAAAGGTGCAGGATGTCG | |
| | TGAACACACAGGGTCAGGCCCTCTCTCATTTGACAGTGCAGC | |
| | TTCAGAATAACTTCCAAGCAATCAGTTCAAGCATCAGCGACA | |
| | TCTACAACCGGCTGGACCCCCCATCTGCAGATGCGCAGGTGG | |
| | ACAGGCTAATCACTGGACGCTTGACGGCACTAAATGCCTTTG | |
| | TCAGCCAAACTCTGACCCGGCAAGCAGAGGTGCGGGCCAGT | |
| | AGACAACTGGCCAAAGACAAGGTCAACGAGTGCGTCAGGTC | |
| | CCAGTCCCAGCGTTTTGGATTCTGTGGGAACGGGACGCACCT | |
| | GTTCTCATTAGCCAATGCTGCACCCAATGGCATGATCTTTTTC | |
| | CATACTGTTCTACTTCCTACTGCCTATGAAACCGTGACCGCTT | |
| | GGAGCGGCATCTGCGCATCTGATGGCGATAGGACCTTCGGGC | |
| | TGGTCGTTAAGGATGTCCAGCTAACGCTGTTCCGGAACTTGG | |
| | ATGACAAGTTCTACCTGACCCCCAGGACCATGTACCAGCCGA | |
| | GAGTGGCAACGAGTTCTGACTTCGTGCAAATTGAGGGCTGT | |
| | GACGTCCTGTTTGTTAATGCAACAGTGATCGATCTGCCCAGT | |
| | ATCATACCAGATTACATAGACATAAACCAGACAGTCCAGGAC | |
| | ATACTGGAGAATTACAGGCCAAACTGGACCGTACCAGAGTTC | |
| | ACGCTGGACATATTCAACGCTACGTACCTCAATTTGACTGGG | |
| | GAAATTGATGACTTGGAGTTCAGGTCGGAGAAGCTCCACAA | |
| | CACCACTGTGGAGCTGGCCATCCTGATTGACAACATCAACAA | |
| | CACTCTGGTGAACCTGGAGTGGCTAAATCGCATTGAAACCTA | |
| | TGTCAAGTGGCCTTGGTACGTTTGGCTACTGATCGGACTCGT | |
| | GGTAGTCTTCTGCATACCACTCCTGCTATTTTGCTGCTTCAGC | |
| | ACAGGGTGCTGTGGCTGCATTGGATGCCTAGGTTCCTGCTGT | |
| | CACAGTATCTGCAGCAGAAGACAATTCGAGAACTACGAGCC | |
| | CATAGAAAAGGTCCACGTACAT | |
| 178 | ATGAGATTTGTGATGTCCCCCACTGTCCTGCTGCTGCTCCTGG | MHCIsp- |
| | GAGCCCTGGCAGCCCCCCAGACCTGGGCAGGCAGCACCACC | S(S2)-MITD |
| | AACAATGAATGCATCCAGGTGAACGTGACCCAGCTGGCAGG | |
| | CAATGAAAATTTGATCAGAGACTTCCTGTTCAGCAACTTCAA | |
| | GGAGGAGGGCAGTGTAGTGGTGGGAGGCTACTACCCAACAG | |
| | AGGTGTGGTACAACTGCAGCAGAACAGCCAGAACCACAGCC | |
| | TTCCAGTACTTCAACAACATCCACGCCTTCTACTTTGTGATGG | |
| | AGGCCATGGAAAACAGCACAGGAAATGCCAGAGGAAAACC | |
| | CCTGCTCTTCCACGTGCACGGAGAGCCCGTGTCAGTCATCAT | |
| | CAGCGCCTACAGAGATGACGTCCAGCAGCGGCCCCTGCTGA | |
| | AGCATGGACTGGTCTGCATCACCAAGAACAGACACATCAAC | |
| | TACGAGCAGTTCACCAGCAACCAGTGGAACAGCACCTGCAC | |
| | AGGAGCAGACAGAAAAATCCCCTTCAGCGTCATCCCCACAG | |
| | ACAACGGCACCAAAATCTATGGCCTGGAGTGGAATGATGACT | |
| | TTGTGACAGCCTATATCAGCGGCAGGAGCTACCACCTCAACA | |
| | TCAACACCAACTGGTTCAACAACGTCACCCTGCTCTACTCCA | |
| | GATCCAGCACAGCCACCTGGGAGTACAGCGCCGCCTATGCCT | |
| | ACCAGGGAGTCTCCAACTTCACCTACTACAAACTGAACAAC | |
| | ACCAACGGCCTGAAAACCTACGAGCTGTGTGAGGACTACGA | |
| | GCACTGCACAGGCTATGCCACAAATGTGTTTGCCCCAACCAG | |
| | CGGAGGCTACATCCCAGACGGCTTCTCCTTCAACAACTGGTT | |
| | CCTCCTCACCAACTCCTCCACATTTGTGAGCGGCAGATTTGT | |
| | GACCAACCAGCCCCTGCTGATCAACTGCCTGTGGCCCGTGCC | |
| | CAGCTTTGGAGTGGCAGCCCAGGAGTTCTGCTTCGAGGGAG | |
| | CCCAGTTCAGCCAGTGCAACGGAGTCAGCCTGAACAACACA | |
| | GTGGACGTGATCAGATTCAACCTGAACTTCACAGCAGACGT | |
| | GCAGAGTGGAATGGGAGCCACCGTCTTCAGCCTGAACACCA | |
| | CAGGAGGAGTGATCCTGGAGATCAGCTGCTACAGCGACACA | |
| | GTGAGCGAGAGCAGCAGCTACAGCTACGGAGAGATCCCATT | |
| | TGGCATCACAGATGGCCCCAGGTACTGCTACGTCCTGTACAA | |
| | TGGAACAGCCCTGAAATACCTGGGCACCCTCCCACCCAGCG | |
| | TGAAGGAGATCGCCATCAGCAAGTGGGGCCACTTCTACATCA | |
| | ATGGCTACAACTTCTTCAGCACCTTCCCCATCGGCTGCATCTC | |
| | CTTCAACCTGACCACAGGAGTGAGCGGGGCCTTCTGGACAA | |
| | TCGCCTACACATCCTACACAGAAGCCCTGGTGCAGGTGGAG | |
| | AACACAGCCATCAAAAACGTCACCTACTGCAACAGCCACAT | |
| | CAACAACATCAAGTGCAGCCAGCTGACAGCCAACCTGAACA | |
| | ACGGCTTCTACCCAGTGGCCAGCTCAGAGGTGGGCTTCGTG | |
| | AACAAGAGCGTGGTGCTCCTGCCCAGCTTCTTCACCTACACA | |
| | GCAGTGAACATCACAATTGACCTGGGCATGAAGCTGAGCGG | |
| | CTACGGCCAGCCAATTGCCAGCACCCTCTCCAACATCACCCT | |
| | CCCCATGCAGGACAATAACACAGATGTGTACTGCATCAGATC | |
| | CAACCAGTTCTCTGTCTACGTGCACAGCACCTGCAAAAGCA | |
| | GCCTGTGGGACAACATCTTCAACCAGGACTGCACAGATGTCC | |
| | TGGAGGCCACAGCCGTGATCAAAACAGGCACCTGCCCCTTC | |
| | AGCTTTGACAAACTCAACAACTACCTTACATTCAACAAATTC | |
| | TGCCTCTCCCTCAGCCCAGTGGGAGCCAACTGCAAGTTTGAT | |
| | GTGGCCGCCAGGACCAGGACAAATGAACAAGTGGTCAGAA | |
| | GCCTCTACGTCATCTACGAGGAGGGAGACAACATCGTGGGG | |
| | GTCCCCAGCGACAACAGCGGCCTGCACGACCTGAGTGTGCT | |
| | CCACCTGGACAGCTGCACAGACTACAACATCTACGGCAGGA | |
| | CTGGGGTGGGCATCATCAGAAGAACCAACAGCACACTGCTG | |
| | AGTGGCCTGTACTACACCAGCCTGAGTGGAGACTTGCTGGG | |
| | CTTCAAGAATGTGTCAGATGGGGTGATCTACAGTGTGACCCC | |
| | CTGTGACGTGTCTGCCCAGGCTGCAGTCATCGACGGAGCCAT | |
| | CGTGGGAGCCATGACCAGCATTAACAGCGAGCTGCTGGGCC | |
| | TGACCCACTGGACCACCACCCCCAACTTCTACTACTACTCCA | |
| | TCTACAACTACACATCAGAAAGAACAAGAGACACAGCCATC | |
| | GACAGCAATGACGTGGACTGTGAGCCAGTCATCACCTACAG | |
| | CAACATCGGAGTGTGCAAGAACGGAGCCCTGGTGTTCATCA | |
| | ACGTGACCCACAGCGACGGAGATGTCCAGCCCATCAGCACA | |
| | GGAAATGTGACCATCCCAACCAACTTCACCATCAGCGTCCAG | |
| | GTGGAATACATGCAGGTGTACACCACCCCAGTGTCCATCGAC | |
| | TGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCCTGGTATGTGTGGCTGCTGATTGG | |
| | ACTGGTGGTGGTGTTCTGCATCCCACTGCTGCTGTTCTGCTG | |
| | CTTCAGCACCGGCTGCTGTGGATGCATCGGCTGCTTGGGCAG | |
| | CTGCTGCCACAGCATCTGCAGCAGGAGGCAGTTTGAGAACT | |
| | ACGAACCAATTGAAAAAGTGCACGTCCACTTCCTGGGCATC | |
| | ATCGCCGGCGTGGTGGTCCTGGTGGTCACAGTGGTGGTGGG | |
| | AGCTGTGATCTGGAGAAAGAAGTGCAGCGGCAGGAAGGGC | |
| | CCAAGCTACAGCCACGCTGCCAGAGATGACTCCACCCAGGG | |
| | CAGCGACAGCAGCCTGATGGCCCCCAAGGTG | |
| 179 | ATGAGATTTGTGATGAGCCCCACTGTGCTGCTGCTGCTGCTG | MHCIsp- |
| | GGAGCCCTGGCAGCCCCCCAGACCTGGGCTGGCTCAACCAC | S(S3)-MITD |
| | CAACAACGAGTGCATCCAGGTGAACGTGACCCAGCTGGCAG | |
| | GCAACGAGAACCTCATCAGAGACTTCCTCTTCTCCAACTTCA | |
| | AGGAGGAGGGCTCAGTGGTGGTCGGCGGCTACTACCCAACA | |
| | GAGGTGTGGTACAACTGCTCAAGGACCGCCAGAACCACAGC | |
| | CTTCCAGTACTTCAACAACATCCACGCCTTCTACTTCGTGATG | |
| | GAGGCCATGGAGAACTCCACCGGGAACGCCAGGGGCAAGC | |
| | CACTACTCTTCCACGTGCACGGAGAGCCAGTGAGCGTGATCA | |
| | TCTCAGCCTACAGGGACGACGTGCAGCAGCGCCCCCTGCTG | |
| | AAGCATGGACTGGTGTGCATCACCAAGAACAGGCACATCAA | |
| | CTACGAGCAGTTCACCAGCAACCAGTGGAACAGCACCTGCA | |
| | CCGGCGCAGACAGGAAGATCCCCTTCTCAGTGATCCCAACA | |
| | GACAACGGAACCAAAATCTACGGCCTGGAGTGGAACGACGA | |
| | CTTCGTGACCGCCTACATCAGCGGCAGGTCCTACCATCTCAA | |
| | CATCAACACCAACTGGTTCAACAACGTCACCCTCCTCTACAG | |
| | CAGGTCATCCACAGCCACCTGGGAGTACTCAGCTGCCTATGC | |
| | ATACCAGGGAGTCTCCAACTTCACATACTACAAACTCAACAA | |
| | CACCAACGGCCTCAAGACCTACGAGCTGTGTGAGGACTACG | |
| | AGCACTGCACCGGCTACGCAACAAACGTCTTCGCCCCAACC | |
| | TCCGGAGGCTACATCCCAGACGGCTTCTCCTTCAACAACTGG | |
| | TTCCTCCTCACAAACAGCTCCACCTTCGTGTCAGGAAGGTTC | |
| | GTGACCAACCAGCCCCTGCTCATCAACTGCCTCTGGCCCGTC | |
| | CCCTCCTTCGGAGTGGCCGCCCAGGAGTTCTGCTTCGAGGG | |
| | AGCCCAGTTCTCCCAGTGCAACGGAGTCTCCCTCAACAACA | |
| | CCGTGGACGTCATCAGATTCAACCTCAACTTCACAGCAGACG | |
| | TCCAGAGCGGCATGGGAGCCACCGTGTTCAGCCTGAACACC | |
| | ACAGGAGGAGTGATCCTGGAGATCTCCTGCTACTCAGACACA | |
| | GTGTCAGAGTCCTCCTCCTACAGCTACGGAGAGATCCCATTC | |
| | GGCATCACAGACGGCCCCAGATACTGCTACGTGCTGTACAAC | |
| | GGCACAGCCCTGAAGTACCTGGGCACCCTCCCCCCATCAGTG | |
| | AAGGAGATCGCCATCAGCAAGTGGGGCCACTTCTACATCAAC | |
| | GGCTACAACTTCTTCTCCACCTTCCCCATCGGCTGCATCAGCT | |
| | TCAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCCTGGTACGTCTGGCTCCTCATCGGCC | |
| | TGGTGGTGGTCTTCTGCATCCCACTGCTGCTGTTCTGCTGCTT | |
| | CTCCACCGGCTGCTGTGGATGCATCGGCTGCCTGGGCTCATG | |
| | CTGCCACTCAATCTGCTCAAGGAGGCAGTTTGAAAACTACGA | |
| | GCCAATAGAAAAAGTCCACGTCCACTTCCTGGGCATAATCGC | |
| | CGGCGTGGTGGTGCTGGTGGTCACAGTGGTGGTCGGAGCAG | |
| | TGATCTGGAGGAAGAAGTGCTCAGGGAGGAAGGGCCCATCC | |
| | TACTCCCACGCCGCCAGGGACGACAGCACCCAGGGCTCAGA | |
| | CTCATCCCTGATGGCCCCCAAGGTG | |
| 180 | ATGCGTTTTGTAATGTCACCTACTGTACTACTACTACTACTCG | MHCIsp- |
| | GAGCACTAGCAGCACCTCAGACTTGGGCCGGATCAACCACA | S(S6)-MITD |
| | AATAACGAGTGCATTCAGGTCAACGTCACCCAGCTGGCCGGT | |
| | AACGAGAACCTAATTAGAGACTTCCTATTCTCGAACTTTAAA | |
| | GAGGAAGGCTCTGTGGTGGTCGGAGGTTACTACCCCACAGA | |
| | AGTGTGGTACAATTGCTCACGTACAGCCAGGACCACTGCCTT | |
| | CCAGTACTTCAACAACATTCATGCCTTCTACTTTGTCATGGAA | |
| | GCCATGGAGAACTCCACTGGGAATGCCAGAGGAAAGCCTCT | |
| | CCTCTTCCATGTCCATGGAGAGCCTGTCTCTGTGATTATCTCA | |
| | GCATATAGGGATGATGTGCAGCAGCGGCCGCTGCTTAAGCAT | |
| | GGCCTAGTGTGCATTACTAAGAACCGACATATCAATTATGAGC | |
| | AGTTCACCTCCAACCAGTGGAACTCCACATGCACTGGTGCTG | |
| | ATAGGAAGATCCCGTTCAGCGTTATCCCCACCGATAATGGCA | |
| | CAAAGATTTATGGCCTAGAATGGAACGATGATTTTGTTACTGC | |
| | CTACATATCAGGAAGAAGTTACCACTTAAACATTAACACCAA | |
| | TTGGTTCAATAATGTTACACTTCTGTACTCTCGCAGCAGTACG | |
| | GCCACTTGGGAGTATTCGGCTGCATATGCCTACCAAGGTGTA | |
| | AGCAACTTCACCTACTACAAGCTGAACAATACGAACGGTCTG | |
| | AAGACTTATGAGCTGTGCGAAGACTACGAGCACTGTACGGG | |
| | CTATGCGACAAATGTCTTCGCCCCGACGAGCGGCGGGTACAT | |
| | ACCGGATGGCTTCTCCTTCAACAACTGGTTCCTCCTTACCAAT | |
| | AGCTCCACTTTCGTATCAGGAAGATTTGTTACGAACCAACCC | |
| | CTTCTCATTAACTGTCTGTGGCCAGTGCCCTCCTTCGGAGTAG | |
| | CTGCTCAAGAGTTCTGTTTCGAGGGTGCACAGTTCAGCCAGT | |
| | GTAATGGAGTGTCGCTGAACAACACTGTGGACGTGATCAGGT | |
| | TTAATTTGAACTTCACAGCTGATGTTCAGTCCGGCATGGGCG | |
| | CGACTGTGTTCAGCCTAAACACCACGGGTGGCGTCATCTTGG | |
| | AGATTAGTTGTTACTCTGACACTGTGTCAGAGAGCAGCAGTT | |
| | ACTCCTACGGAGAAATTCCTTTCGGCATCACAGACGGTCCCC | |
| | GGTACTGCTATGTGCTGTACAACGGAACTGCTTTGAAGTACC | |
| | TGGGGACATTGCCACCTTCTGTGAAGGAAATAGCCATCTCTA | |
| | AGTGGGGTCACTTTTACATTAACGGCTATAATTTCTTTTCCAC | |
| | TTTCCCAATTGGATGCATTAGCTTCAACCTGACAACAGGTGT | |
| | GTCTGGAGCCTTCTGGACCATCGCCTATACCTCTTACACAGA | |
| | GGCTCTAGTACAGGTGGAGAACACAGCTATAAAGAACGTGA | |
| | CGTACTGTAACAGTCACATAAACAATATCAAGTGTTCTCAGTT | |
| | GACTGCGAACTTAAACAATGGGTTTTATCCAGTGGCGAGCTC | |
| | GGAGGTGGGGTTTGTAAACAAATCTGTGGTGCTGTTGCCCTC | |
| | CTTCTTCACGTACACTGCAGTGAACATCACCATTGATTTGGG | |
| | GATGAAACTGTCCGGCTACGGGCAGCCTATAGCATCTACACT | |
| | GAGCAATATCACACTGCCCATGCAGGATAACAATACAGATGT | |
| | GTACTGTATCCGCTCAAACCAGTTCTCTGTATACGTGCACAGT | |
| | ACATGCAAGAGCTCGCTATGGGACAACATTTTCAACCAGGAT | |
| | TGTACTGATGTGCTTGAAGCAACTGCAGTGATCAAAACAGGC | |
| | ACATGCCCGTTCAGCTTTGATAAGCTCAACAACTACCTAACG | |
| | TTCAACAAGTTCTGCTTGAGCCTGTCTCCAGTAGGCGCCAAT | |
| | TGCAAGTTTGACGTTGCAGCGCGAACACGGACAAACGAACA | |
| | GGTAGTGCGGTCGCTCTATGTTATCTACGAGGAGGGGGACAA | |
| | CATAGTCGGGGTTCCATCCGACAACTCAGGTTTGCACGACCT | |
| | GAGTGTGCTCCATTTGGACTCATGCACGGATTATAACATCTAC | |
| | GGGCGCACAGGTGTGGGGATAATACGAAGAACAAACTCTAC | |
| | GCTATTGAGCGGGCTCTACTACACCTCATTGAGTGGGGACCT | |
| | GCTAGGGTTCAAGAACGTATCTGACGGTGTGATCTATAGCGT | |
| | CACACCATGTGACGTATCAGCCCAAGCTGCTGTGATTGACGG | |
| | GGCGATTGTGGGGGCTATGACTTCAATTAACAGCGAGCTCCT | |
| | AGGCCTGACCCACTGGACTACCACCCCAAACTTCTACTACTA | |
| | CAGCATTTATAACTATACCAGTGAGCGCACCAGGGACACTGC | |
| | CATTGACAGCAATGACGTCGACTGCGAGCCTGTTATTACCTA | |
| | CAGCAACATCGGTGTTTGTAAGAATGGAGCTCTAGTCTTCAT | |
| | AAACGTAACGCACTCTGATGGCGATGTTCAACCAATTTCCAC | |
| | TGGGAACGTAACCATACCCACCAACTTTACTATTTCCGTCCAG | |
| | GTGGAGTACATGCAAGTATATACCACGCCAGTGTCCATCGAC | |
| | TGCGCTCGGTATGTGTGCAACGGTAACCCACGCTGCAATAAG | |
| | CTGCTAACGCAGTACGTCAGCGCCTGCCAGACAATAGAGCA | |
| | GGCATTGGCAATGGGTGCAAGGCTTGAAAACATGGAGGTGG | |
| | ACTCCATGTTGTTCGTGTCTGAAAACGCTCTTAAACTAGCATC | |
| | CGTGGAGGCATTCAACAGTACTGAGAACTTGGACTCTATCTA | |
| | TAAGGAGTGGCCCTCCATTGGGGGCAGCTGGCTTGGAGGTCT | |
| | AAAAGACATCCTGCCCAGCCACAACTCCAAGAGGAAGTACG | |
| | GGTCCGCTATAGAGGACCTCCTCTTTGACAAGGTTGTTACTT | |
| | CTGGTCTTGGCACAGTGGACGAAGACTACAAGAGGTGCACA | |
| | GGAGGCTATGATATAGCTGACCTGGTGTGTGCTCAATACTACA | |
| | ACGGTATAATGGTTCTCCCAGGTGTGGCCAACGCTGACAAGA | |
| | TGACAATGTACACAGCCTCTTTAGCTGGAGGCATTACCCTGG | |
| | GAGCCCTTGGGGGTGGCGCAGTGGCAATTCCATTTGCCGTTG | |
| | CGGTGCAGGCCCGACTAAACTATGTCGCACTTCAAACAGATG | |
| | TGCTCAACAAGAACCAACAAATACTGGCCAACGCTTTCAAC | |
| | CAGGCCATTGGTAACATTACGCAGGCATTTGGCAAGGTGAAT | |
| | GACGCCATCCACCAGACCAGCCAGGGACTTGCCACAGTGGC | |
| | CAAGGCCTTGGCAAAGGTGCAGGATGTCGTGAACACACAGG | |
| | GTCAGGCCCTCTCTCATTTGACAGTGCAGCTTCAGAATAACT | |
| | TCCAAGCAATCAGTTCAAGCATCAGCGACATCTACAACCGGC | |
| | TGGACCCCCCATCTGCAGATGCGCAGGTGGACAGGCTAATCA | |
| | CTGGACGCTTGACGGCACTAAATGCCTTTGTCAGCCAAACTC | |
| | TGACCCGGCAAGCAGAGGTGCGGGCCAGTAGACAACTGGCC | |
| | AAAGACAAGGTCAACGAGTGCGTCAGGTCCCAGTCCCAGCG | |
| | TTTTGGATTCTGTGGGAACGGGACGCACCTGTTCTCATTAGC | |
| | CAATGCTGCACCCAATGGCATGATCTTTTTCCATACTGTTCTA | |
| | CTTCCTACTGCCTATGAAACCGTGACCGCTTGGAGCGGCATC | |
| | TGCGCATCTGATGGCGATAGGACCTTCGGGCTGGTCGTTAAG | |
| | GATGTCCAGCTAACGCTGTTCCGGAACTTGGATGACAAGTTC | |
| | TACCTGACCCCCAGGACCATGTACCAGCCGAGAGTGGCAAC | |
| | GAGTTCTGACTTCGTGCAAATTGAGGGCTGTGACGTCCTGTT | |
| | TGTTAATGCAACAGTGATCGATCTGCCCAGTATCATACCAGAT | |
| | TACATAGACATAAACCAGACAGTCCAGGACATACTGGAGAAT | |
| | TACAGGCCAAACTGGACCGTACCAGAGTTCACGCTGGACAT | |
| | ATTCAACGCTACGTACCTCAATTTGACTGGGGAAATTGATGA | |
| | CTTGGAGTTCAGGTCGGAGAAGCTCCACAACACCACTGTGG | |
| | AGCTGGCCATCCTGATTGACAACATCAACAACACTCTGGTGA | |
| | ACCTGGAGTGGCTAAATCGCATTGAAACCTATGTCAAGTGGC | |
| | CTTGGTACGTTTGGCTACTGATCGGACTCGTGGTAGTCTTCTG | |
| | CATACCACTCCTGCTATTTTGCTGCTTCAGCACAGGGTGCTGT | |
| | GGCTGCATTGGATGCCTAGGTTCCTGCTGTCACAGTATCTGCA | |
| | GCAGAAGACAATTCGAGAACTACGAGCCCATAGAAAAGGTC | |
| | CACGTACATTTCCTGGGGATAATCGCAGGAGTGGTTGTTCTA | |
| | GTGGTGACCGTGGTAGTTGGGGCAGTGATCTGGAGAAAGAA | |
| | ATGCTCTGGCCGTAAGGGACCATCCTACTCCCATGCAGCACG | |
| | TGATGATTCTACCCAGGGCAGCGACAGTTCATTGATGGCCCC | |
| | TAAAGTC | |
| 181 | ATGAGATTTGTGATGTCCCCCACTGTCCTGCTGCTGCTCCTGG | MHCIsp- |
| | GAGCCCTGGCAGCCCCCCAGACCTGGGCAGGCAGCACCACC | S_ec(S2)- |
| | AACAATGAATGCATCCAGGTGAACGTGACCCAGCTGGCAGG | MITD |
| | CAATGAAAATTTGATCAGAGACTTCCTGTTCAGCAACTTCAA | |
| | GGAGGAGGGCAGTGTAGTGGTGGGAGGCTACTACCCAACAG | |
| | AGGTGTGGTACAACTGCAGCAGAACAGCCAGAACCACAGCC | |
| | TTCCAGTACTTCAACAACATCCACGCCTTCTACTTTGTGATGG | |
| | AGGCCATGGAAAACAGCACAGGAAATGCCAGAGGAAAACC | |
| | CCTGCTCTTCCACGTGCACGGAGAGCCCGTGTCAGTCATCAT | |
| | CAGCGCCTACAGAGATGACGTCCAGCAGCGGCCCCTGCTGA | |
| | AGCATGGACTGGTCTGCATCACCAAGAACAGACACATCAAC | |
| | TACGAGCAGTTCACCAGCAACCAGTGGAACAGCACCTGCAC | |
| | AGGAGCAGACAGAAAAATCCCCTTCAGCGTCATCCCCACAG | |
| | ACAACGGCACCAAAATCTATGGCCTGGAGTGGAATGATGACT | |
| | TTGTGACAGCCTATATCAGCGGCAGGAGCTACCACCTCAACA | |
| | TCAACACCAACTGGTTCAACAACGTCACCCTGCTCTACTCCA | |
| | GATCCAGCACAGCCACCTGGGAGTACAGCGCCGCCTATGCCT | |
| | ACCAGGGAGTCTCCAACTTCACCTACTACAAACTGAACAAC | |
| | ACCAACGGCCTGAAAACCTACGAGCTGTGTGAGGACTACGA | |
| | GCACTGCACAGGCTATGCCACAAATGTGTTTGCCCCAACCAG | |
| | CGGAGGCTACATCCCAGACGGCTTCTCCTTCAACAACTGGTT | |
| | CCTCCTCACCAACTCCTCCACATTTGTGAGCGGCAGATTTGT | |
| | GACCAACCAGCCCCTGCTGATCAACTGCCTGTGGCCCGTGCC | |
| | CAGCTTTGGAGTGGCAGCCCAGGAGTTCTGCTTCGAGGGAG | |
| | CCCAGTTCAGCCAGTGCAACGGAGTCAGCCTGAACAACACA | |
| | GTGGACGTGATCAGATTCAACCTGAACTTCACAGCAGACGT | |
| | GCAGAGTGGAATGGGAGCCACCGTCTTCAGCCTGAACACCA | |
| | CAGGAGGAGTGATCCTGGAGATCAGCTGCTACAGCGACACA | |
| | GTGAGCGAGAGCAGCAGCTACAGCTACGGAGAGATCCCATT | |
| | TGGCATCACAGATGGCCCCAGGTACTGCTACGTCCTGTACAA | |
| | TGGAACAGCCCTGAAATACCTGGGCACCCTCCCACCCAGCG | |
| | TGAAGGAGATCGCCATCAGCAAGTGGGGCCACTTCTACATCA | |
| | ATGGCTACAACTTCTTCAGCACCTTCCCCATCGGCTGCATCTC | |
| | CTTCAACCTGACCACAGGAGTGAGCGGGGCCTTCTGGACAA | |
| | TCGCCTACACATCCTACACAGAAGCCCTGGTGCAGGTGGAG | |
| | AACACAGCCATCAAAAACGTCACCTACTGCAACAGCCACAT | |
| | CAACAACATCAAGTGCAGCCAGCTGACAGCCAACCTGAACA | |
| | ACGGCTTCTACCCAGTGGCCAGCTCAGAGGTGGGCTTCGTG | |
| | AACAAGAGCGTGGTGCTCCTGCCCAGCTTCTTCACCTACACA | |
| | GCAGTGAACATCACAATTGACCTGGGCATGAAGCTGAGCGG | |
| | CTACGGCCAGCCAATTGCCAGCACCCTCTCCAACATCACCCT | |
| | CCCCATGCAGGACAATAACACAGATGTGTACTGCATCAGATC | |
| | CAACCAGTTCTCTGTCTACGTGCACAGCACCTGCAAAAGCA | |
| | GCCTGTGGGACAACATCTTCAACCAGGACTGCACAGATGTCC | |
| | TGGAGGCCACAGCCGTGATCAAAACAGGCACCTGCCCCTTC | |
| | AGCTTTGACAAACTCAACAACTACCTTACATTCAACAAATTC | |
| | TGCCTCTCCCTCAGCCCAGTGGGAGCCAACTGCAAGTTTGAT | |
| | GTGGCCGCCAGGACCAGGACAAATGAACAAGTGGTCAGAA | |
| | GCCTCTACGTCATCTACGAGGAGGGAGACAACATCGTGGGG | |
| | GTCCCCAGCGACAACAGCGGCCTGCACGACCTGAGTGTGCT | |
| | CCACCTGGACAGCTGCACAGACTACAACATCTACGGCAGGA | |
| | CTGGGGTGGGCATCATCAGAAGAACCAACAGCACACTGCTG | |
| | AGTGGCCTGTACTACACCAGCCTGAGTGGAGACTTGCTGGG | |
| | CTTCAAGAATGTGTCAGATGGGGTGATCTACAGTGTGACCCC | |
| | CTGTGACGTGTCTGCCCAGGCTGCAGTCATCGACGGAGCCAT | |
| | CGTGGGAGCCATGACCAGCATTAACAGCGAGCTGCTGGGCC | |
| | TGACCCACTGGACCACCACCCCCAACTTCTACTACTACTCCA | |
| | TCTACAACTACACATCAGAAAGAACAAGAGACACAGCCATC | |
| | GACAGCAATGACGTGGACTGTGAGCCAGTCATCACCTACAG | |
| | CAACATCGGAGTGTGCAAGAACGGAGCCCTGGTGTTCATCA | |
| | ACGTGACCCACAGCGACGGAGATGTCCAGCCCATCAGCACA | |
| | GGAAATGTGACCATCCCAACCAACTTCACCATCAGCGTCCAG | |
| | GTGGAATACATGCAGGTGTACACCACCCCAGTGTCCATCGAC | |
| | TGTGCCAGATACGTGTGCAATGGAAACCCCAGATGCAACAA | |
| | GCTCCTCACCCAGTACGTGTCAGCCTGCCAGACAATCGAGCA | |
| | GGCCCTGGCCATGGGAGCCAGGCTCGAGAACATGGAAGTGG | |
| | ACAGCATGCTGTTTGTCTCAGAGAATGCCCTGAAACTGGCCA | |
| | GCGTGGAGGCCTTCAACAGCACAGAGAACCTGGACAGCATC | |
| | TACAAGGAGTGGCCATCAATCGGAGGCAGCTGGCTGGGAGG | |
| | ACTTAAGGACATCCTGCCAAGCCACAACAGCAAAAGAAAGT | |
| | ACGGCAGCGCCATTGAGGACCTGCTGTTTGACAAGGTGGTC | |
| | ACCTCCGGCCTGGGCACAGTGGATGAGGACTACAAGAGATG | |
| | CACCGGCGGCTATGACATTGCCGACCTGGTGTGTGCCCAGTA | |
| | CTACAATGGCATCATGGTGCTGCCTGGAGTGGCCAACGCCGA | |
| | CAAAATGACCATGTACACCGCCTCCCTGGCTGGAGGCATCAC | |
| | ACTGGGAGCCCTGGGGGGAGGAGCAGTGGCCATCCCCTTTG | |
| | CAGTGGCTGTGCAGGCCAGACTCAACTACGTGGCCCTGCAG | |
| | ACAGACGTGCTCAACAAGAACCAGCAGATCCTGGCCAACGC | |
| | TTTCAACCAGGCTATCGGAAACATCACCCAGGCCTTTGGAAA | |
| | AGTGAATGATGCCATCCACCAGACCAGCCAGGGCCTGGCCA | |
| | CAGTGGCCAAGGCCCTGGCCAAGGTGCAGGACGTGGTCAAC | |
| | ACCCAGGGCCAGGCCCTCAGTCACCTCACAGTACAGCTCCA | |
| | GAACAACTTCCAGGCAATCTCCTCCTCCATCAGCGACATCTA | |
| | CAACAGGCTGGACCCCCCAAGCGCTGATGCCCAGGTGGACA | |
| | GACTGATCACAGGAAGACTCACAGCCCTCAACGCATTTGTGT | |
| | CCCAGACACTGACCAGGCAGGCAGAGGTCAGGGCCAGCAG | |
| | GCAGCTGGCCAAGGACAAGGTGAATGAGTGCGTGAGGAGCC | |
| | AGAGCCAGAGATTTGGCTTCTGCGGAAACGGCACCCACCTG | |
| | TTCAGCCTGGCCAACGCCGCCCCCAACGGCATGATTTTCTTC | |
| | CACACAGTCCTCCTCCCCACAGCCTACGAAACAGTGACAGC | |
| | CTGGTCAGGCATCTGTGCCAGCGACGGAGACAGAACCTTTG | |
| | GCCTGGTGGTGAAGGATGTGCAGCTCACCCTCTTCAGAAAC | |
| | CTGGATGACAAGTTCTACCTCACCCCAAGAACCATGTACCAG | |
| | CCCAGAGTGGCCACAAGCAGCGACTTTGTGCAGATTGAGGG | |
| | CTGTGACGTGCTGTTTGTGAATGCAACAGTGATTGACCTCCC | |
| | AAGCATCATCCCAGATTACATCGACATCAACCAGACAGTGCA | |
| | GGACATCCTGGAGAACTACAGGCCCAACTGGACAGTGCCAG | |
| | AGTTCACCCTGGACATCTTCAACGCCACCTACCTGAACCTGA | |
| | CAGGAGAAATTGACGACCTGGAGTTCAGATCAGAAAAACTT | |
| | CACAACACCACCGTGGAGCTTGCCATCCTCATTGACAACATT | |
| | AACAACACACTGGTCAACCTGGAATGGCTGAACAGAATTGA | |
| | AACCTACGTGAAGTGGCCCTTCCTGGGCATCATCGCCGGCGT | |
| | GGTGGTCCTGGTGGTCACAGTGGTGGTGGGAGCTGTGATCT | |
| | GGAGAAAGAAGTGCAGCGGCAGGAAGGGCCCAAGCTACAG | |
| | CCACGCTGCCAGAGATGACTCCACCCAGGGCAGCGACAGCA | |
| | GCCTGATGGCCCCCAAGGTG | |
| 182 | ATGAGATTTGTGATGAGCCCCACTGTGCTGCTGCTGCTGCTG | MHCIsp- |
| | GGAGCCCTGGCAGCCCCCCAGACCTGGGCTGGCTCAACCAC | S_ec(S3)- |
| | CAACAACGAGTGCATCCAGGTGAACGTGACCCAGCTGGCAG | MITD |
| | GCAACGAGAACCTCATCAGAGACTTCCTCTTCTCCAACTTCA | |
| | AGGAGGAGGGCTCAGTGGTGGTCGGCGGCTACTACCCAACA | |
| | GAGGTGTGGTACAACTGCTCAAGGACCGCCAGAACCACAGC | |
| | CTTCCAGTACTTCAACAACATCCACGCCTTCTACTTCGTGATG | |
| | GAGGCCATGGAGAACTCCACCGGGAACGCCAGGGGCAAGC | |
| | CACTACTCTTCCACGTGCACGGAGAGCCAGTGAGCGTGATCA | |
| | TCTCAGCCTACAGGGACGACGTGCAGCAGCGCCCCCTGCTG | |
| | AAGCATGGACTGGTGTGCATCACCAAGAACAGGCACATCAA | |
| | CTACGAGCAGTTCACCAGCAACCAGTGGAACAGCACCTGCA | |
| | CCGGCGCAGACAGGAAGATCCCCTTCTCAGTGATCCCAACA | |
| | GACAACGGAACCAAAATCTACGGCCTGGAGTGGAACGACGA | |
| | CTTCGTGACCGCCTACATCAGCGGCAGGTCCTACCATCTCAA | |
| | CATCAACACCAACTGGTTCAACAACGTCACCCTCCTCTACAG | |
| | CAGGTCATCCACAGCCACCTGGGAGTACTCAGCTGCCTATGC | |
| | ATACCAGGGAGTCTCCAACTTCACATACTACAAACTCAACAA | |
| | CACCAACGGCCTCAAGACCTACGAGCTGTGTGAGGACTACG | |
| | AGCACTGCACCGGCTACGCAACAAACGTCTTCGCCCCAACC | |
| | TCCGGAGGCTACATCCCAGACGGCTTCTCCTTCAACAACTGG | |
| | TTCCTCCTCACAAACAGCTCCACCTTCGTGTCAGGAAGGTTC | |
| | GTGACCAACCAGCCCCTGCTCATCAACTGCCTCTGGCCCGTC | |
| | CCCTCCTTCGGAGTGGCCGCCCAGGAGTTCTGCTTCGAGGG | |
| | AGCCCAGTTCTCCCAGTGCAACGGAGTCTCCCTCAACAACA | |
| | CCGTGGACGTCATCAGATTCAACCTCAACTTCACAGCAGACG | |
| | TCCAGAGCGGCATGGGAGCCACCGTGTTCAGCCTGAACACC | |
| | ACAGGAGGAGTGATCCTGGAGATCTCCTGCTACTCAGACACA | |
| | GTGTCAGAGTCCTCCTCCTACAGCTACGGAGAGATCCCATTC | |
| | GGCATCACAGACGGCCCCAGATACTGCTACGTGCTGTACAAC | |
| | GGCACAGCCCTGAAGTACCTGGGCACCCTCCCCCCATCAGTG | |
| | AAGGAGATCGCCATCAGCAAGTGGGGCCACTTCTACATCAAC | |
| | GGCTACAACTTCTTCTCCACCTTCCCCATCGGCTGCATCAGCT | |
| | TCAACCTGACCACCGGAGTGTCCGGAGCCTTCTGGACCATCG | |
| | CCTACACATCATACACCGAGGCCCTGGTGCAGGTGGAGAAC | |
| | ACAGCCATAAAGAACGTGACCTACTGCAACAGCCACATCAA | |
| | CAACATCAAGTGCTCCCAGCTGACAGCCAACCTGAACAACG | |
| | GCTTCTACCCAGTGGCCTCCAGCGAGGTGGGCTTCGTGAACA | |
| | AGAGCGTGGTCCTACTCCCCTCCTTCTTCACCTACACAGCAG | |
| | TCAACATCACAATTGACCTGGGCATGAAGCTGTCCGGCTACG | |
| | GCCAGCCAATCGCCAGCACCCTGTCCAACATCACCCTGCCAA | |
| | TGCAGGACAACAACACCGACGTCTACTGCATCAGAAGCAAC | |
| | CAGTTCTCCGTGTACGTCCACTCCACCTGCAAGTCCTCCCTC | |
| | TGGGACAACATCTTCAACCAGGACTGCACAGACGTGCTGGA | |
| | GGCCACAGCTGTGATCAAGACAGGAACCTGCCCTTTCTCATT | |
| | CGACAAGCTCAACAACTACCTGACCTTCAACAAGTTCTGCCT | |
| | GAGCCTGTCCCCAGTGGGAGCCAACTGCAAGTTCGACGTGG | |
| | CCGCCAGAACCAGGACCAACGAGCAGGTGGTCAGAAGCCT | |
| | GTACGTCATCTACGAGGAGGGAGACAACATCGTGGGAGTGC | |
| | CCAGCGACAACTCAGGCCTGCACGACCTGAGCGTGCTGCAC | |
| | CTGGACTCCTGCACAGACTACAACATCTACGGCAGGACAGG | |
| | AGTGGGCATCATCAGGAGGACCAACAGCACACTGCTGTCCG | |
| | GCCTCTACTACACCTCCCTGTCCGGAGACTTGCTGGGATTCA | |
| | AGAACGTGTCAGACGGAGTCATCTACAGCGTCACCCCATGTG | |
| | ACGTGAGCGCCCAGGCAGCAGTGATAGACGGAGCCATCGTG | |
| | GGAGCCATGACCTCAATCAACTCAGAACTGCTGGGCCTCACC | |
| | CACTGGACAACAACACCCAACTTCTACTACTACTCCATCTAC | |
| | AACTACACATCAGAAAGAACAAGGGACACAGCAATCGACTC | |
| | CAACGACGTGGACTGTGAGCCAGTCATCACCTACTCCAACAT | |
| | CGGCGTGTGCAAGAACGGAGCCCTGGTGTTCATCAACGTCA | |
| | CCCACTCAGACGGCGACGTCCAGCCAATCTCCACAGGAAAC | |
| | GTCACCATCCCCACCAACTTCACCATCAGCGTGCAGGTGGAG | |
| | TACATGCAGGTCTACACCACCCCAGTCTCCATCGACTGTGCC | |
| | AGGTACGTGTGCAACGGCAACCCAAGATGCAACAAACTGCT | |
| | GACCCAGTACGTGAGCGCCTGCCAGACCATCGAGCAGGCCC | |
| | TGGCCATGGGCGCCAGGCTGGAGAACATGGAGGTGGACAGC | |
| | ATGCTCTTTGTGAGCGAGAACGCCCTGAAGCTTGCCAGCGTG | |
| | GAGGCCTTCAACAGCACCGAAAACCTGGACTCCATCTACAA | |
| | AGAGTGGCCCTCCATAGGAGGCTCCTGGCTGGGAGGCCTGA | |
| | AGGACATCCTCCCATCCCACAACAGCAAAAGAAAGTACGGC | |
| | AGCGCCATCGAAGACCTGCTGTTCGACAAGGTGGTCACCTC | |
| | AGGACTGGGCACAGTGGACGAGGACTACAAGAGGTGCACC | |
| | GGAGGCTACGACATCGCAGACCTGGTCTGTGCCCAGTACTAC | |
| | AACGGCATCATGGTGCTCCCAGGCGTGGCCAACGCCGACAA | |
| | GATGACCATGTACACAGCAAGCCTGGCTGGAGGAATCACACT | |
| | GGGAGCCCTGGGAGGAGGGGCCGTGGCCATTCCATTCGCCG | |
| | TGGCCGTGCAGGCCAGACTGAACTACGTGGCCCTGCAGACA | |
| | GACGTGCTAAACAAGAACCAGCAGATCCTGGCCAACGCCTT | |
| | CAACCAGGCCATCGGCAACATCACCCAGGCCTTCGGCAAGG | |
| | TGAACGACGCAATCCACCAGACATCACAGGGCCTGGCAACA | |
| | GTGGCCAAGGCCCTGGCCAAGGTCCAGGACGTGGTGAACAC | |
| | CCAGGGCCAGGCCCTCTCACACCTGACAGTCCAGCTGCAGA | |
| | ACAACTTCCAGGCAATCTCCTCCTCCATCTCAGACATCTACA | |
| | ACAGACTGGACCCCCCCTCAGCCGACGCCCAGGTGGACAGA | |
| | CTCATCACAGGCAGGCTGACCGCCCTCAACGCCTTCGTGTCC | |
| | CAGACCCTCACCAGGCAGGCCGAGGTGAGGGCCAGCAGGC | |
| | AGCTCGCCAAGGACAAGGTGAACGAGTGCGTCAGAAGCCA | |
| | GAGCCAGAGGTTCGGCTTCTGTGGCAACGGCACCCACCTGT | |
| | TCTCCCTGGCCAACGCAGCCCCCAACGGCATGATCTTCTTCC | |
| | ACACAGTCCTCCTCCCAACAGCATATGAGACAGTCACCGCCT | |
| | GGTCAGGAATCTGTGCCTCAGACGGGGACAGAACCTTCGGC | |
| | CTGGTGGTCAAGGACGTGCAGCTGACACTCTTCAGAAACCT | |
| | GGACGACAAATTCTACCTGACCCCCAGGACCATGTACCAGCC | |
| | AAGGGTGGCCACCTCCTCAGACTTCGTGCAGATCGAGGGCT | |
| | GTGACGTGCTCTTCGTGAACGCCACCGTCATCGACCTCCCAT | |
| | CCATCATCCCAGACTACATCGACATCAACCAGACAGTGCAGG | |
| | ACATCCTGGAGAACTACCGCCCCAACTGGACCGTGCCAGAG | |
| | TTCACCCTAGACATATTCAACGCCACCTACCTGAACCTGACA | |
| | GGAGAAATTGACGACCTGGAGTTCAGATCAGAAAAGCTACA | |
| | CAACACCACCGTGGAGTTAGCCATCCTCATAGACAACATTAA | |
| | CAACACCCTCGTCAACCTGGAGTGGCTCAACAGGATTGAAA | |
| | CCTACGTGAAGTGGCCCTTCCTGGGCATAATCGCCGGCGTGG | |
| | TGGTGCTGGTGGTCACAGTGGTGGTCGGAGCAGTGATCTGG | |
| | AGGAAGAAGTGCTCAGGGAGGAAGGGCCCATCCTACTCCCA | |
| | CGCCGCCAGGGACGACAGCACCCAGGGCTCAGACTCATCCC | |
| | TGATGGCCCCCAAGGTG | |
| 183 | ATGCGTTTTGTAATGTCACCTACTGTACTACTACTACTACTCG | MHCIsp- |
| | GAGCACTAGCAGCACCTCAGACTTGGGCCGGATCAACCACA | S_ec(S6)- |
| | AATAACGAGTGCATTCAGGTCAACGTCACCCAGCTGGCCGGT | MITD |
| | AACGAGAACCTAATTAGAGACTTCCTATTCTCGAACTTTAAA | |
| | GAGGAAGGCTCTGTGGTGGTCGGAGGTTACTACCCCACAGA | |
| | AGTGTGGTACAATTGCTCACGTACAGCCAGGACCACTGCCTT | |
| | CCAGTACTTCAACAACATTCATGCCTTCTACTTTGTCATGGAA | |
| | GCCATGGAGAACTCCACTGGGAATGCCAGAGGAAAGCCTCT | |
| | CCTCTTCCATGTCCATGGAGAGCCTGTCTCTGTGATTATCTCA | |
| | GCATATAGGGATGATGTGCAGCAGCGGCCGCTGCTTAAGCAT | |
| | GGCCTAGTGTGCATTACTAAGAACCGACATATCAATTATGAGC | |
| | AGTTCACCTCCAACCAGTGGAACTCCACATGCACTGGTGCTG | |
| | ATAGGAAGATCCCGTTCAGCGTTATCCCCACCGATAATGGCA | |
| | CAAAGATTTATGGCCTAGAATGGAACGATGATTTTGTTACTGC | |
| | CTACATATCAGGAAGAAGTTACCACTTAAACATTAACACCAA | |
| | TTGGTTCAATAATGTTACACTTCTGTACTCTCGCAGCAGTACG | |
| | GCCACTTGGGAGTATTCGGCTGCATATGCCTACCAAGGTGTA | |
| | AGCAACTTCACCTACTACAAGCTGAACAATACGAACGGTCTG | |
| | AAGACTTATGAGCTGTGCGAAGACTACGAGCACTGTACGGG | |
| | CTATGCGACAAATGTCTTCGCCCCGACGAGCGGCGGGTACAT | |
| | ACCGGATGGCTTCTCCTTCAACAACTGGTTCCTCCTTACCAAT | |
| | AGCTCCACTTTCGTATCAGGAAGATTTGTTACGAACCAACCC | |
| | CTTCTCATTAACTGTCTGTGGCCAGTGCCCTCCTTCGGAGTAG | |
| | CTGCTCAAGAGTTCTGTTTCGAGGGTGCACAGTTCAGCCAGT | |
| | GTAATGGAGTGTCGCTGAACAACACTGTGGACGTGATCAGGT | |
| | TTAATTTGAACTTCACAGCTGATGTTCAGTCCGGCATGGGCG | |
| | CGACTGTGTTCAGCCTAAACACCACGGGTGGCGTCATCTTGG | |
| | AGATTAGTTGTTACTCTGACACTGTGTCAGAGAGCAGCAGTT | |
| | ACTCCTACGGAGAAATTCCTTTCGGCATCACAGACGGTCCCC | |
| | GGTACTGCTATGTGCTGTACAACGGAACTGCTTTGAAGTACC | |
| | TGGGGACATTGCCACCTTCTGTGAAGGAAATAGCCATCTCTA | |
| | AGTGGGGTCACTTTTACATTAACGGCTATAATTTCTTTTCCAC | |
| | TTTCCCAATTGGATGCATTAGCTTCAACCTGACAACAGGTGT | |
| | GTCTGGAGCCTTCTGGACCATCGCCTATACCTCTTACACAGA | |
| | GGCTCTAGTACAGGTGGAGAACACAGCTATAAAGAACGTGA | |
| | CGTACTGTAACAGTCACATAAACAATATCAAGTGTTCTCAGTT | |
| | GACTGCGAACTTAAACAATGGGTTTTATCCAGTGGCGAGCTC | |
| | GGAGGTGGGGTTTGTAAACAAATCTGTGGTGCTGTTGCCCTC | |
| | CTTCTTCACGTACACTGCAGTGAACATCACCATTGATTTGGG | |
| | GATGAAACTGTCCGGCTACGGGCAGCCTATAGCATCTACACT | |
| | GAGCAATATCACACTGCCCATGCAGGATAACAATACAGATGT | |
| | GTACTGTATCCGCTCAAACCAGTTCTCTGTATACGTGCACAGT | |
| | ACATGCAAGAGCTCGCTATGGGACAACATTTTCAACCAGGAT | |
| | TGTACTGATGTGCTTGAAGCAACTGCAGTGATCAAAACAGGC | |
| | ACATGCCCGTTCAGCTTTGATAAGCTCAACAACTACCTAACG | |
| | TTCAACAAGTTCTGCTTGAGCCTGTCTCCAGTAGGCGCCAAT | |
| | TGCAAGTTTGACGTTGCAGCGCGAACACGGACAAACGAACA | |
| | GGTAGTGCGGTCGCTCTATGTTATCTACGAGGAGGGGGACAA | |
| | CATAGTCGGGGTTCCATCCGACAACTCAGGTTTGCACGACCT | |
| | GAGTGTGCTCCATTTGGACTCATGCACGGATTATAACATCTAC | |
| | GGGCGCACAGGTGTGGGGATAATACGAAGAACAAACTCTAC | |
| | GCTATTGAGCGGGCTCTACTACACCTCATTGAGTGGGGACCT | |
| | GCTAGGGTTCAAGAACGTATCTGACGGTGTGATCTATAGCGT | |
| | CACACCATGTGACGTATCAGCCCAAGCTGCTGTGATTGACGG | |
| | GGCGATTGTGGGGGCTATGACTTCAATTAACAGCGAGCTCCT | |
| | AGGCCTGACCCACTGGACTACCACCCCAAACTTCTACTACTA | |
| | CAGCATTTATAACTATACCAGTGAGCGCACCAGGGACACTGC | |
| | CATTGACAGCAATGACGTCGACTGCGAGCCTGTTATTACCTA | |
| | CAGCAACATCGGTGTTTGTAAGAATGGAGCTCTAGTCTTCAT | |
| | AAACGTAACGCACTCTGATGGCGATGTTCAACCAATTTCCAC | |
| | TGGGAACGTAACCATACCCACCAACTTTACTATTTCCGTCCAG | |
| | GTGGAGTACATGCAAGTATATACCACGCCAGTGTCCATCGAC | |
| | TGCGCTCGGTATGTGTGCAACGGTAACCCACGCTGCAATAAG | |
| | CTGCTAACGCAGTACGTCAGCGCCTGCCAGACAATAGAGCA | |
| | GGCATTGGCAATGGGTGCAAGGCTTGAAAACATGGAGGTGG | |
| | ACTCCATGTTGTTCGTGTCTGAAAACGCTCTTAAACTAGCATC | |
| | CGTGGAGGCATTCAACAGTACTGAGAACTTGGACTCTATCTA | |
| | TAAGGAGTGGCCCTCCATTGGGGGCAGCTGGCTTGGAGGTCT | |
| | AAAAGACATCCTGCCCAGCCACAACTCCAAGAGGAAGTACG | |
| | GGTCCGCTATAGAGGACCTCCTCTTTGACAAGGTTGTTACTT | |
| | CTGGTCTTGGCACAGTGGACGAAGACTACAAGAGGTGCACA | |
| | GGAGGCTATGATATAGCTGACCTGGTGTGTGCTCAATACTACA | |
| | ACGGTATAATGGTTCTCCCAGGTGTGGCCAACGCTGACAAGA | |
| | TGACAATGTACACAGCCTCTTTAGCTGGAGGCATTACCCTGG | |
| | GAGCCCTTGGGGGTGGCGCAGTGGCAATTCCATTTGCCGTTG | |
| | CGGTGCAGGCCCGACTAAACTATGTCGCACTTCAAACAGATG | |
| | TGCTCAACAAGAACCAACAAATACTGGCCAACGCTTTCAAC | |
| | CAGGCCATTGGTAACATTACGCAGGCATTTGGCAAGGTGAAT | |
| | GACGCCATCCACCAGACCAGCCAGGGACTTGCCACAGTGGC | |
| | CAAGGCCTTGGCAAAGGTGCAGGATGTCGTGAACACACAGG | |
| | GTCAGGCCCTCTCTCATTTGACAGTGCAGCTTCAGAATAACT | |
| | TCCAAGCAATCAGTTCAAGCATCAGCGACATCTACAACCGGC | |
| | TGGACCCCCCATCTGCAGATGCGCAGGTGGACAGGCTAATCA | |
| | CTGGACGCTTGACGGCACTAAATGCCTTTGTCAGCCAAACTC | |
| | TGACCCGGCAAGCAGAGGTGCGGGCCAGTAGACAACTGGCC | |
| | AAAGACAAGGTCAACGAGTGCGTCAGGTCCCAGTCCCAGCG | |
| | TTTTGGATTCTGTGGGAACGGGACGCACCTGTTCTCATTAGC | |
| | CAATGCTGCACCCAATGGCATGATCTTTTTCCATACTGTTCTA | |
| | CTTCCTACTGCCTATGAAACCGTGACCGCTTGGAGCGGCATC | |
| | TGCGCATCTGATGGCGATAGGACCTTCGGGCTGGTCGTTAAG | |
| | GATGTCCAGCTAACGCTGTTCCGGAACTTGGATGACAAGTTC | |
| | TACCTGACCCCCAGGACCATGTACCAGCCGAGAGTGGCAAC | |
| | GAGTTCTGACTTCGTGCAAATTGAGGGCTGTGACGTCCTGTT | |
| | TGTTAATGCAACAGTGATCGATCTGCCCAGTATCATACCAGAT | |
| | TACATAGACATAAACCAGACAGTCCAGGACATACTGGAGAAT | |
| | TACAGGCCAAACTGGACCGTACCAGAGTTCACGCTGGACAT | |
| | ATTCAACGCTACGTACCTCAATTTGACTGGGGAAATTGATGA | |
| | CTTGGAGTTCAGGTCGGAGAAGCTCCACAACACCACTGTGG | |
| | AGCTGGCCATCCTGATTGACAACATCAACAACACTCTGGTGA | |
| | ACCTGGAGTGGCTAAATCGCATTGAAACCTATGTCAAGTGGC | |
| | CTTTCCTGGGGATAATCGCAGGAGTGGTTGTTCTAGTGGTGA | |
| | CCGTGGTAGTTGGGGCAGTGATCTGGAGAAAGAAATGCTCT | |
| | GGCCGTAAGGGACCATCCTACTCCCATGCAGCACGTGATGAT | |
| | TCTACCCAGGGCAGCGACAGTTCATTGATGGCCCCTAAAGTC | |
| 184 | ATGAGATTTGTGATGTCCCCCACTGTCCTGCTGCTGCTCCTGG | MHCIsp- |
| | GAGCCCTGGCAGCCCCCCAGACCTGGGCAGGCAGCTGTGAG | SII(S2)- |
| | CCAGTCATCACCTACAGCAACATCGGAGTGTGCAAGAACGG | MITD |
| | AGCCCTGGTGTTCATCAACGTGACCCACAGCGACGGAGATG | |
| | TCCAGCCCATCAGCACAGGAAATGTGACCATCCCAACCAACT | |
| | TCACCATCAGCGTCCAGGTGGAATACATGCAGGTGTACACCA | |
| | CCCCAGTGTCCATCGACTGTGCCAGATACGTGTGCAATGGAA | |
| | ACCCCAGATGCAACAAGCTCCTCACCCAGTACGTGTCAGCCT | |
| | GCCAGACAATCGAGCAGGCCCTGGCCATGGGAGCCAGGCTC | |
| | GAGAACATGGAAGTGGACAGCATGCTGTTTGTCTCAGAGAA | |
| | TGCCCTGAAACTGGCCAGCGTGGAGGCCTTCAACAGCACAG | |
| | AGAACCTGGACAGCATCTACAAGGAGTGGCCATCAATCGGA | |
| | GGCAGCTGGCTGGGAGGACTTAAGGACATCCTGCCAAGCCA | |
| | CAACAGCAAAAGAAAGTACGGCAGCGCCATTGAGGACCTGC | |
| | TGTTTGACAAGGTGGTCACCTCCGGCCTGGGCACAGTGGAT | |
| | GAGGACTACAAGAGATGCACCGGCGGCTATGACATTGCCGA | |
| | CCTGGTGTGTGCCCAGTACTACAATGGCATCATGGTGCTGCC | |
| | TGGAGTGGCCAACGCCGACAAAATGACCATGTACACCGCCT | |
| | CCCTGGCTGGAGGCATCACACTGGGAGCCCTGGGGGGAGGA | |
| | GCAGTGGCCATCCCCTTTGCAGTGGCTGTGCAGGCCAGACTC | |
| | AACTACGTGGCCCTGCAGACAGACGTGCTCAACAAGAACCA | |
| | GCAGATCCTGGCCAACGCTTTCAACCAGGCTATCGGAAACAT | |
| | CACCCAGGCCTTTGGAAAAGTGAATGATGCCATCCACCAGAC | |
| | CAGCCAGGGCCTGGCCACAGTGGCCAAGGCCCTGGCCAAGG | |
| | TGCAGGACGTGGTCAACACCCAGGGCCAGGCCCTCAGTCAC | |
| | CTCACAGTACAGCTCCAGAACAACTTCCAGGCAATCTCCTCC | |
| | TCCATCAGCGACATCTACAACAGGCTGGACCCCCCAAGCGCT | |
| | GATGCCCAGGTGGACAGACTGATCACAGGAAGACTCACAGC | |
| | CCTCAACGCATTTGTGTCCCAGACACTGACCAGGCAGGCAG | |
| | AGGTCAGGGCCAGCAGGCAGCTGGCCAAGGACAAGGTGAA | |
| | TGAGTGCGTGAGGAGCCAGAGCCAGAGATTTGGCTTCTGCG | |
| | GAAACGGCACCCACCTGTTCAGCCTGGCCAACGCCGCCCCC | |
| | AACGGCATGATTTTCTTCCACACAGTCCTCCTCCCCACAGCC | |
| | TACGAAACAGTGACAGCCTGGTCAGGCATCTGTGCCAGCGA | |
| | CGGAGACAGAACCTTTGGCCTGGTGGTGAAGGATGTGCAGC | |
| | TCACCCTCTTCAGAAACCTGGATGACAAGTTCTACCTCACCC | |
| | CAAGAACCATGTACCAGCCCAGAGTGGCCACAAGCAGCGAC | |
| | TTTGTGCAGATTGAGGGCTGTGACGTGCTGTTTGTGAATGCA | |
| | ACAGTGATTGACCTCCCAAGCATCATCCCAGATTACATCGAC | |
| | ATCAACCAGACAGTGCAGGACATCCTGGAGAACTACAGGCC | |
| | CAACTGGACAGTGCCAGAGTTCACCCTGGACATCTTCAACG | |
| | CCACCTACCTGAACCTGACAGGAGAAATTGACGACCTGGAG | |
| | TTCAGATCAGAAAAACTTCACAACACCACCGTGGAGCTTGC | |
| | CATCCTCATTGACAACATTAACAACACACTGGTCAACCTGGA | |
| | ATGGCTGAACAGAATTGAAACCTACGTGAAGTGGCCCTGGTA | |
| | TGTGTGGCTGCTGATTGGACTGGTGGTGGTGTTCTGCATCCC | |
| | ACTGCTGCTGTTCTGCTGCTTCAGCACCGGCTGCTGTGGATG | |
| | CATCGGCTGCTTGGGCAGCTGCTGCCACAGCATCTGCAGCAG | |
| | GAGGCAGTTTGAGAACTACGAACCAATTGAAAAAGTGCACG | |
| | TCCACTTCCTGGGCATCATCGCCGGCGTGGTGGTCCTGGTGG | |
| | TCACAGTGGTGGTGGGAGCTGTGATCTGGAGAAAGAAGTGC | |
| | AGCGGCAGGAAGGGCCCAAGCTACAGCCACGCTGCCAGAG | |
| | ATGACTCCACCCAGGGCAGCGACAGCAGCCTGATGGCCCCC | |
| | AAGGTG | |
| 185 | ATGAGATTTGTGATGAGCCCCACTGTGCTGCTGCTGCTGCTG | MHCIsp- |
| | GGAGCCCTGGCAGCCCCCCAGACCTGGGCTGGCTCATGTGA | SII(S3)- |
| | GCCAGTCATCACCTACTCCAACATCGGCGTGTGCAAGAACGG | MITD |
| | AGCCCTGGTGTTCATCAACGTCACCCACTCAGACGGCGACGT | |
| | CCAGCCAATCTCCACAGGAAACGTCACCATCCCCACCAACTT | |
| | CACCATCAGCGTGCAGGTGGAGTACATGCAGGTCTACACCAC | |
| | CCCAGTCTCCATCGACTGTGCCAGGTACGTGTGCAACGGCAA | |
| | CCCAAGATGCAACAAACTGCTGACCCAGTACGTGAGCGCCT | |
| | GCCAGACCATCGAGCAGGCCCTGGCCATGGGCGCCAGGCTG | |
| | GAGAACATGGAGGTGGACAGCATGCTCTTTGTGAGCGAGAA | |
| | CGCCCTGAAGCTTGCCAGCGTGGAGGCCTTCAACAGCACCG | |
| | AAAACCTGGACTCCATCTACAAAGAGTGGCCCTCCATAGGAG | |
| | GCTCCTGGCTGGGAGGCCTGAAGGACATCCTCCCATCCCACA | |
| | ACAGCAAAAGAAAGTACGGCAGCGCCATCGAAGACCTGCTG | |
| | TTCGACAAGGTGGTCACCTCAGGACTGGGCACAGTGGACGA | |
| | GGACTACAAGAGGTGCACCGGAGGCTACGACATCGCAGACC | |
| | TGGTCTGTGCCCAGTACTACAACGGCATCATGGTGCTCCCAG | |
| | GCGTGGCCAACGCCGACAAGATGACCATGTACACAGCAAGC | |
| | CTGGCTGGAGGAATCACACTGGGAGCCCTGGGAGGAGGGGC | |
| | CGTGGCCATTCCATTCGCCGTGGCCGTGCAGGCCAGACTGAA | |
| | CTACGTGGCCCTGCAGACAGACGTGCTAAACAAGAACCAGC | |
| | AGATCCTGGCCAACGCCTTCAACCAGGCCATCGGCAACATCA | |
| | CCCAGGCCTTCGGCAAGGTGAACGACGCAATCCACCAGACA | |
| | TCACAGGGCCTGGCAACAGTGGCCAAGGCCCTGGCCAAGGT | |
| | CCAGGACGTGGTGAACACCCAGGGCCAGGCCCTCTCACACC | |
| | TGACAGTCCAGCTGCAGAACAACTTCCAGGCAATCTCCTCCT | |
| | CCATCTCAGACATCTACAACAGACTGGACCCCCCCTCAGCCG | |
| | ACGCCCAGGTGGACAGACTCATCACAGGCAGGCTGACCGCC | |
| | CTCAACGCCTTCGTGTCCCAGACCCTCACCAGGCAGGCCGA | |
| | GGTGAGGGCCAGCAGGCAGCTCGCCAAGGACAAGGTGAAC | |
| | GAGTGCGTCAGAAGCCAGAGCCAGAGGTTCGGCTTCTGTGG | |
| | CAACGGCACCCACCTGTTCTCCCTGGCCAACGCAGCCCCCA | |
| | ACGGCATGATCTTCTTCCACACAGTCCTCCTCCCAACAGCAT | |
| | ATGAGACAGTCACCGCCTGGTCAGGAATCTGTGCCTCAGAC | |
| | GGGGACAGAACCTTCGGCCTGGTGGTCAAGGACGTGCAGCT | |
| | GACACTCTTCAGAAACCTGGACGACAAATTCTACCTGACCCC | |
| | CAGGACCATGTACCAGCCAAGGGTGGCCACCTCCTCAGACTT | |
| | CGTGCAGATCGAGGGCTGTGACGTGCTCTTCGTGAACGCCA | |
| | CCGTCATCGACCTCCCATCCATCATCCCAGACTACATCGACAT | |
| | CAACCAGACAGTGCAGGACATCCTGGAGAACTACCGCCCCA | |
| | ACTGGACCGTGCCAGAGTTCACCCTAGACATATTCAACGCCA | |
| | CCTACCTGAACCTGACAGGAGAAATTGACGACCTGGAGTTC | |
| | AGATCAGAAAAGCTACACAACACCACCGTGGAGTTAGCCAT | |
| | CCTCATAGACAACATTAACAACACCCTCGTCAACCTGGAGTG | |
| | GCTCAACAGGATTGAAACCTACGTGAAGTGGCCCTGGTACGT | |
| | CTGGCTCCTCATCGGCCTGGTGGTGGTCTTCTGCATCCCACT | |
| | GCTGCTGTTCTGCTGCTTCTCCACCGGCTGCTGTGGATGCATC | |
| | GGCTGCCTGGGCTCATGCTGCCACTCAATCTGCTCAAGGAGG | |
| | CAGTTTGAAAACTACGAGCCAATAGAAAAAGTCCACGTCCA | |
| | CTTCCTGGGCATAATCGCCGGCGTGGTGGTGCTGGTGGTCAC | |
| | AGTGGTGGTCGGAGCAGTGATCTGGAGGAAGAAGTGCTCAG | |
| | GGAGGAAGGGCCCATCCTACTCCCACGCCGCCAGGGACGAC | |
| | AGCACCCAGGGCTCAGACTCATCCCTGATGGCCCCCAAGGT | |
| | G | |
| 186 | ATGCGTTTTGTAATGTCACCTACTGTACTACTACTACTACTCG | MHCIsp- |
| | GAGCACTAGCAGCACCTCAGACTTGGGCCGGATCATGCGAG | SII(S6)- |
| | CCTGTTATTACCTACAGCAACATCGGTGTTTGTAAGAATGGAG | MITD |
| | CTCTAGTCTTCATAAACGTAACGCACTCTGATGGCGATGTTCA | |
| | ACCAATTTCCACTGGGAACGTAACCATACCCACCAACTTTAC | |
| | TATTTCCGTCCAGGTGGAGTACATGCAAGTATATACCACGCCA | |
| | GTGTCCATCGACTGCGCTCGGTATGTGTGCAACGGTAACCCA | |
| | CGCTGCAATAAGCTGCTAACGCAGTACGTCAGCGCCTGCCAG | |
| | ACAATAGAGCAGGCATTGGCAATGGGTGCAAGGCTTGAAAA | |
| | CATGGAGGTGGACTCCATGTTGTTCGTGTCTGAAAACGCTCT | |
| | TAAACTAGCATCCGTGGAGGCATTCAACAGTACTGAGAACTT | |
| | GGACTCTATCTATAAGGAGTGGCCCTCCATTGGGGGCAGCTG | |
| | GCTTGGAGGTCTAAAAGACATCCTGCCCAGCCACAACTCCA | |
| | AGAGGAAGTACGGGTCCGCTATAGAGGACCTCCTCTTTGACA | |
| | AGGTTGTTACTTCTGGTCTTGGCACAGTGGACGAAGACTACA | |
| | AGAGGTGCACAGGAGGCTATGATATAGCTGACCTGGTGTGTG | |
| | CTCAATACTACAACGGTATAATGGTTCTCCCAGGTGTGGCCA | |
| | ACGCTGACAAGATGACAATGTACACAGCCTCTTTAGCTGGAG | |
| | GCATTACCCTGGGAGCCCTTGGGGGTGGCGCAGTGGCAATTC | |
| | CATTTGCCGTTGCGGTGCAGGCCCGACTAAACTATGTCGCAC | |
| | TTCAAACAGATGTGCTCAACAAGAACCAACAAATACTGGCC | |
| | AACGCTTTCAACCAGGCCATTGGTAACATTACGCAGGCATTT | |
| | GGCAAGGTGAATGACGCCATCCACCAGACCAGCCAGGGACT | |
| | TGCCACAGTGGCCAAGGCCTTGGCAAAGGTGCAGGATGTCG | |
| | TGAACACACAGGGTCAGGCCCTCTCTCATTTGACAGTGCAGC | |
| | TTCAGAATAACTTCCAAGCAATCAGTTCAAGCATCAGCGACA | |
| | TCTACAACCGGCTGGACCCCCCATCTGCAGATGCGCAGGTGG | |
| | ACAGGCTAATCACTGGACGCTTGACGGCACTAAATGCCTTTG | |
| | TCAGCCAAACTCTGACCCGGCAAGCAGAGGTGCGGGCCAGT | |
| | AGACAACTGGCCAAAGACAAGGTCAACGAGTGCGTCAGGTC | |
| | CCAGTCCCAGCGTTTTGGATTCTGTGGGAACGGGACGCACCT | |
| | GTTCTCATTAGCCAATGCTGCACCCAATGGCATGATCTTTTTC | |
| | CATACTGTTCTACTTCCTACTGCCTATGAAACCGTGACCGCTT | |
| | GGAGCGGCATCTGCGCATCTGATGGCGATAGGACCTTCGGGC | |
| | TGGTCGTTAAGGATGTCCAGCTAACGCTGTTCCGGAACTTGG | |
| | ATGACAAGTTCTACCTGACCCCCAGGACCATGTACCAGCCGA | |
| | GAGTGGCAACGAGTTCTGACTTCGTGCAAATTGAGGGCTGT | |
| | GACGTCCTGTTTGTTAATGCAACAGTGATCGATCTGCCCAGT | |
| | ATCATACCAGATTACATAGACATAAACCAGACAGTCCAGGAC | |
| | ATACTGGAGAATTACAGGCCAAACTGGACCGTACCAGAGTTC | |
| | ACGCTGGACATATTCAACGCTACGTACCTCAATTTGACTGGG | |
| | GAAATTGATGACTTGGAGTTCAGGTCGGAGAAGCTCCACAA | |
| | CACCACTGTGGAGCTGGCCATCCTGATTGACAACATCAACAA | |
| | CACTCTGGTGAACCTGGAGTGGCTAAATCGCATTGAAACCTA | |
| | TGTCAAGTGGCCTTGGTACGTTTGGCTACTGATCGGACTCGT | |
| | GGTAGTCTTCTGCATACCACTCCTGCTATTTTGCTGCTTCAGC | |
| | ACAGGGTGCTGTGGCTGCATTGGATGCCTAGGTTCCTGCTGT | |
| | CACAGTATCTGCAGCAGAAGACAATTCGAGAACTACGAGCC | |
| | CATAGAAAAGGTCCACGTACATTTCCTGGGGATAATCGCAGG | |
| | AGTGGTTGTTCTAGTGGTGACCGTGGTAGTTGGGGCAGTGAT | |
| | CTGGAGAAAGAAATGCTCTGGCCGTAAGGGACCATCCTACTC | |
| | CCATGCAGCACGTGATGATTCTACCCAGGGCAGCGACAGTTC | |
| | ATTGATGGCCCCTAAAGTC | |
| 187 | ATGCTGGTCTTCCTGCATGCTGTGCTGGTGACTGTGCTCATCC | 7a |
| | TGCCCCTCATCGGCCGCATCCAGCTGCTGGAGAGACTTCTCC | |
| | TGAGCCACCTGCTGAACCTGACCACAGTCAGCAATGTCCTG | |
| | GGGGTCCCAGACAGCAGCCTGCGGGTCAACTGCCTGCAGCT | |
| | GCTGAAGCCAGACTGCCTGGACTTCAACATCCTGCACAAGG | |
| | TGCTGGCAGAAACACGGCTGCTGGTGGTGGTGCTGCGGGTC | |
| | ATCTTCCTGGTGCTGCTGGGCTTCAGCTGCTACACCCTGCTG | |
| | GGGGCCCTCTTC | |
| 188 | ATGGATGCTGTGAAGAGCATCGGCATCTCTGTGGATGCTGTG | 3a |
| | CTGGATGAGCTGGACAGCATTGCCTTTGCTGTCACCCTGAAG | |
| | GTGCTCTTCAACAGCGGGAAGCTGCTGGTGTGCATCGGCTTC | |
| | GGGGACACCTTTGAGGAGGCTGAGCAGAAGGCCTATGCCAA | |
| | GAGCAAGCTGGTG | |
| 189 | ATGAGATTTGTGATGTCCCCCACTGTCCTGCTGCTGCTCCTGG | MHCIsp- |
| | GAGCCCTGGCAGCCCCCCAGACCTGGGCAGGCAGCGAGAG | M(HF1902)- |
| | ATACTGTGCCATGAAGGACGACAGCAGCAACACCTGCATCA | MITD |
| | ACGGCACCAACAGCAGCTGCCAGACCTGCTTTGAGAGAGGG | |
| | GACCTGATCTGGCACCTGGCCAACTGGAACTTCAGCTGGAG | |
| | CGTGATCCTGATCGTGTTCATCACCGTGCTGCAGTATGGAAG | |
| | ACCCCAGTTCAGCTGGCTGGTGTACGGCATCAAGATGCTGAT | |
| | CATGTGGCTGCTGTGGCCCATCGTGCTGGCCCTGACCATCTT | |
| | CAACGCCTACAGCGAGTACCAGGTGTCCAGATACGTGATGTT | |
| | TGGCTTCAGCATCGCCGGGGCCGTGGTGACCTTCGCCCTGTG | |
| | CATGATGTACTTCGTGAGGTCCATCCAGCTGTACAGGAGGAC | |
| | AAAGTCATGGTGGTCCTTCAACCCAGAAACCAATGCCATCCT | |
| | GTGCGTCAACGCACTGGGCAGAAGCTACGTCCTACCACTGG | |
| | ACGGCACTCCTACAGGAGTGACCCTGACCCTGCTGTCAGGC | |
| | AATCTGTACGCAGAGGGGTTCAAGATGGCCGGTGGCCTGAC | |
| | CATCGAGCATCTGCCTAAGTACGTGATGATCGCCACCCCTAG | |
| | CAGGACAATCGTGTACACCCTGGTGGGAAAGCAGCTAAAGG | |
| | CGACCACAGCCACAGGCTGGGCCTACTACGTGAAGTCCAAG | |
| | GCAGGGGACTATTCAACCGAGGCCAGGACCGACAACCTGTC | |
| | AGAGCACGAGAAGCTGCTGCACATGGTCTTCCTGGGCATCAT | |
| | CGCCGGCGTGGTGGTCCTGGTGGTCACAGTGGTGGTGGGAG | |
| | CTGTGATCTGGAGAAAGAAGTGCAGCGGCAGGAAGGGCCCA | |
| | AGCTACAGCCACGCTGCCAGAGATGACTCCACCCAGGGCAG | |
| | CGACAGCAGCCTGATGGCCCCCAAGGTG | |
| 190 | ATGGCTACACAAGGACAGAGAGTTAACTGGGGAGATGAACC | N(HF1902) |
| | AAGCAAGAGAAGAGACAGAAGCAACAGCAGAGGAAGAAA | |
| | AAATGGAAACATCCCCCTGTCCTACTTCAACCCCATCACCCT | |
| | GGAGAGCGGCAGCAAGTTCTGGAACATCTGCCCCAGGGACT | |
| | TTGTGCCCAAGGGCATTGGAAACAAGGACCAGCAGATCGGC | |
| | TACTGGAACAGACAGGTGCGCTACAGAATTGTGCGGGGCCA | |
| | GAGGAAGGAGCTGCCCGAGAGATGGTTCTTCTACTTCTCTGG | |
| | AACAGGCCCCCACGCTGATGCCAAGTTCAAGGACAAGATTG | |
| | ATGGAGTGTTCTGGGTGGCCAGAGATGGAGCCATGAACAAG | |
| | CCCACCACCCTGGGCACCAGAGGCACCAACAACGAGAGCA | |
| | AGCCCCTGAAGTTTGATGGCAAGATCCCCCCCCAGTTCCAGC | |
| | TGGAGGTGAACAGAAGCAGAAACAACAGCAGAAGCGGCAG | |
| | CCAGCCCAGGAGCGTGTCCAGAAGCAGAAGCCAGAGCAGA | |
| | GGAAGACAGCAGAGCAACAACCAGAACACCAACGTGGAGG | |
| | ACACCATCGTGGCCGTGCTGAGCAAGCTGGGCGTGACAGAC | |
| | AAGCAGAGGAGCAGAAGCAAGTCTGGAGAAAGAAACCAGA | |
| | GCAAGCCCAGAGACACCACCCCCAAGAATGCCAACAAGCAC | |
| | ACCTGGAAGAAGACAGCTGGCAAGGGGGACGTGACCAACT | |
| | TCTATGGAGCCAGGAGCAGCAGCGCCAACTTTGGAGACAGC | |
| | GACCTGGTGGCCAATGGAAATGCCGCCAAGTGCTACCCCCA | |
| | GATCGCCGAGTGTGTGCCCAGCGCCAGCAGCATCCTGTTTGG | |
| | CAGCCAGTGGAGCGCCGAGGAGGCCGGGGACCAGGTGAAG | |
| | GTGACCCTGACCCACACCTACTACCTGCCCAAGGATGATGCC | |
| | AAGACCAGCCAGTTCCTGGAGCAGATTGATGCCTACAAGAG | |
| | GCCCAGCGAGGTGGCCAAGGACCAGAGGCAGAGGAAGAGC | |
| | AGAAGCAAGTCTGCTGACAAGAAGCCAGAGGAGCTGTCGGT | |
| | GACCCTCGTCGAGGCATACACCGATGTCTTCGACGACACTCA | |
| | GGTGGAGATGATCGACGAGGTCACCAAC | |
| 191 | ATGAGATTTGTGATGTCCCCCACTGTCCTGCTGCTGCTCCTGG | MHCIsp- |
| | GAGCCCTGGCAGCCCCCCAGACCTGGGCAGGCAGCTGCAGC | SII(HF1902)- |
| | AGCGTGATCACCTACAGCAGCTTCGCCATCTGCAACACAGGA | MITD |
| | GAGATCAAGTACGTGAACGTGACCCACGTGGAGACTGTGGA | |
| | CGACAACATCGGGGTGATCAAGCCCATCAGCACCGGCAACA | |
| | TCACCATCCCCAAGAACTTCACAGTGGCCGTGCAGGCCGAG | |
| | TACATCCAGATCCAGGTGAAGCCTGTGGTGGTGGACTGCGCC | |
| | AAGTACGTGTGCAATGGAAATGGCCACTGCCTGAACCTGCTG | |
| | ACCCAGTACACCTCTGCCTGCCAGACCATCGAGAACGCCCTG | |
| | AACCTGGGCGCCAGACTGGAGTCCCTGATGCTGTCTGAGATG | |
| | GTGACCGTGAGCGAGAGAAACCTGGACCTGGCCACCGTGGA | |
| | GAAGTTCAACAGCACAGTGCTGGGCGGGGAGAAGCTGGGA | |
| | GGCTTCTACTTTGACGGCCTGAAGAGCCTGCTGCCTCCCACC | |
| | ATCGGCAAGAGAAGCGCCGTGGAGGACCTGCTGTTCAACAA | |
| | GGTGGTGACCAGCGGCCTGGGGACCGTGGATGATGACTACA | |
| | AGAAGTGCAGCGCCGGCACAGATGTGGCCGACCTGGCCTGT | |
| | GCCCAGTACTACAACGGCATCATGGTGCTGCCTGGAGTGGTG | |
| | GACCAGAACAAGATGGCCATGTACACCGCCAGCCTGATTGG | |
| | AGGCATGGCCCTGGGCAGCATCACCAGCGCCGTGGCCGTGC | |
| | CCTTCGCCATGCAGGTGCAGGCCAGACTGAACTACGTGGCCC | |
| | TGCAGACAGATGTGCTGCAGGAGAACCAGAAGATCCTGGCC | |
| | AACGCCTTCAACAACGCCATCGGCAACATCACCCTGGCCCTG | |
| | GGGAAGGTGAGCAACAGCATCACCACCATCTCTGGAGGCTT | |
| | CCACACCATGGCCAGCGCCCTGACCAAGATCCAGAGCGTGG | |
| | TGAACCAGCAGGGGGAGGCCCTGTCCCAGCTGACCAGCCAG | |
| | CTGCAGAAGAACTTCCAGGCCATCTCCTCTTCCATTGCCGAG | |
| | ATCTACAACAGACTGGAGAAGGCTGAGGCCGACGCCCAGGT | |
| | GGACAGGCTGATCACAGGAAGACTGGCCGCCCTGAACGCCT | |
| | ACGTGTCCCAGACCCTGACCCAGTATGCCGAGGTGAAGGCC | |
| | AGCAGACAGCTGGCCATGGAGAAGGTGAATGAGTGTGTGAA | |
| | GAGCCAGTCTGACAGATACGGCTTCTGTGGAAATGGAACCC | |
| | ACCTGTTCAGCCTGGTGAACTCTGCCCCTGACGGCCTGCTGT | |
| | TCTTCCACACCGTGCTGCTGCCCACAGAGTGGGAGGAGGTG | |
| | ACAGCCTGGAGTGGCATCTGTGTGAATGACACCTACGCCTAC | |
| | GTGCTGAAAGACTTTGACTACAGCATCTTCAGCTACAACGGC | |
| | ACCTACATGGTGACCCCCAGAAACATGTTCCAGCCCAGAAA | |
| | GCCCCAGATGTCAGACTTCGTGCAGATCACCAGATGCGAGGT | |
| | GACCTTCCTGAACACAACCTACACCACCTTCCAGGAGATCGT | |
| | GATCGACTACATCGACATCAACAAGACCATCGCCGACATGCT | |
| | GGAGCAGTACAACCTGAACTACACAACCCCTGAGCTGAACC | |
| | TGCAGCTGGAGATCTTCAACCAGACCAAGCTGAACCTGACC | |
| | GCCGAGATCGACCAGCTGGAGCAGAGAGCCGACAACCTGAC | |
| | CAACATCGCCCACGAGCTGCAGCAGTACATCGACAACCTGA | |
| | ACAAGACCCTGGTGGACCTGGAGTGGCTGAACAGAATTGAA | |
| | ACCTACGTGAAGTGGCCCTGGTACGTGTGGCTGCTGATCGGC | |
| | CTGGTGATCGTGTTCTGCATCCCTCTGCTGCTGTTCTGCTGCC | |
| | TGAGCACCGGCTGCTGTGGCTGCTTCGGCTTCCTGGGCTCCT | |
| | GCTGCCACTCCCTGTGCAGCAGGAGGCAGTTTGAGTCCTACG | |
| | AGCCCATCGAGAAGGTGCACATCCACTTCCTGGGCATCATCG | |
| | CCGGCGTGGTGGTCCTGGTGGTCACAGTGGTGGTGGGAGCT | |
| | GTGATCTGGAGAAAGAAGTGCAGCGGCAGGAAGGGCCCAA | |
| | GCTACAGCCACGCTGCCAGAGATGACTCCACCCAGGGCAGC | |
| | GACAGCAGCCTGATGGCCCCCAAGGTG | |
| 192 | ATGAGATTTGTGATGTCCCCCACTGTCCTGCTGCTGCTCCTGG | MHCIsp- |
| | GAGCCCTGGCAGCCCCCCAGACCTGGGCAGGCAGCGAGAG | M(SH2211)- |
| | ATACTGTGCCATGCAGGACAGCGGCCTGCAGTGCATCAACGG | MITD |
| | CACCAACAGCAGATGCCAGACCTGCTTTGAAAGAGGGGACC | |
| | TGATCTGGCACCTGGCCAACTGGAACTTCTCCTGGAGCGTGA | |
| | TCCTGATCGTGTTCATCACCGTGCTGCAGTACGGCAGACCAC | |
| | AGTTCTCATGGCTTGTCTATGGCATCAAGATGCTGATTATGTG | |
| | GCTGCTTTGGCCTATCGTCCTGGCCCTGACCATCTTCAACGCC | |
| | TACTCTGAGTACCAGGTGTCAAGGTATGTCATGTTCGGCTTCT | |
| | CAGTGGCTGGAGCTGTGGTGACCTTTGCTCTGTGGATGATGT | |
| | ACTTCGTGAGGTCCGTGCAGCTGTACAGGAGGACAAAGTCA | |
| | TGGTGGTCCTTCAACCCAGAAACCAATGCCATCCTGTGCGTC | |
| | AACGCACTGGGCAGAAGCTACGTCCTACCACTGGACGGCAC | |
| | TCCTACAGGAGTGACCCTGACCCTGCTGTCAGGCAATCTGTA | |
| | CGCAGAGGGGTTCAAGATGGCCGGTGGCCTGACCATCGAGC | |
| | ATCTGCCTAAGTACGTGATGATCGCCACCCCTAGCAGGACAA | |
| | TCGTGTACACCCTGGTGGGAAAGCAGCTAAAGGCGACCACA | |
| | GCCACAGGCTGGGCCTACTACGTGAAGTCCAAGGCAGGGGA | |
| | CTATTCAACCGAGGCCAGGACAGACAACCTGAGCGAGCACG | |
| | AGAAGCTGCTGCACATGGTGTTCCTGGGCATCATCGCCGGCG | |
| | TGGTGGTCCTGGTGGTCACAGTGGTGGTGGGAGCTGTGATCT | |
| | GGAGAAAGAAGTGCAGCGGCAGGAAGGGCCCAAGCTACAG | |
| | CCACGCTGCCAGAGATGACTCCACCCAGGGCAGCGACAGCA | |
| | GCCTGATGGCCCCCAAGGTG | |
| 193 | ATGGCTACACAAGGACAGAGAGTTAACTGGGGAGATGAACC | N(SH2211) |
| | AAGCAAGAGAAGAGGAAGAAGCAACAGCAGAGGAAGAAA | |
| | GAACAATGACATCCCCCTGTCCTTCTACAACCCCATCACCCT | |
| | GGAGCAGGGCAGCAAGTTCTGGAACCTGTGCCCCAGGGACC | |
| | TGGTGCCCAAGGGCATCGGCAACAAGGACCAGCAGATTGGC | |
| | TACTGGAACAGACAGATCAGATACAGAATTGTGAAGGGCCA | |
| | GAGGAAGGAGCTGGCCGAGAGGTGGTTCTTCTACTTCCTGG | |
| | GCACCGGCCCCCATGCTGATGCCAAGTTCAAGGACAAGATTG | |
| | ATGGAGTGTTCTGGGTGGCCAGAGATGGGGCCATGAACAAG | |
| | CCCACCACCCTGGGCACCAGGGGCACCAACAATGAGAGCAA | |
| | GCCCCTGAGGTTTGATGGCAAGATCCCCCCCCAGTTCCAGCT | |
| | GGAGGTGAACAGAAGCAGAAACAACAGCAGAAGCGGCAGC | |
| | CAGAGCAGAAGTGTGTCCAGAAACAGAAGCCAGAGCAGAG | |
| | GAAGACACCACAGCAACAACCAGAACAACAATGTGGAGGA | |
| | CACCATCGTGGCCGTGCTGGAGAAGCTGGGGGTGACAGACA | |
| | AGCAGAGGAGCAGAAGCAAGCCCAGAGAAAGAAGTGACAG | |
| | CAAGCCCAGGGACACCACCCCCAAGAATGCCAACAAGCACA | |
| | CCTGGAAGAAGACAGCTGGAAAAGGAGATGTGACCACCTTC | |
| | TATGGAGCCAGGAGCAGCAGCGCCAACTTTGGAGACAGTGA | |
| | CCTGGTGGCCAATGGAAATGCTGCCAAGTGCTACCCCCAGAT | |
| | TGCTGAGTGTGTGCCCTCTGTGAGCAGCATCATCTTTGGCAG | |
| | CCAGTGGTCAGCAGAGGAGGCTGGGGACCAGGTGAAGGTG | |
| | ACCCTGACCCACACCTACTACCTGCCCAAGGATGATGCCAAG | |
| | ACCAGCCAGTTCCTGGAGCAGATTGATGCCTACAAGAGGCCC | |
| | AGCGAGGTGGCCAAGGACCAGAGGCAGAGGAGGAGCCTGA | |
| | GCAAGTCTGCTGACAAGAAGCCAGAGGAGCTGTCGGTGACC | |
| | CTCGTCGAGGCGTACACCGACGTCTTCGACGACACTCAGGT | |
| | GGAGATGATCGACGAGGTCACCAAC | |
| 194 | ATGAGATTTGTGATGTCCCCCACTGTCCTGCTGCTGCTCCTGG | MHCIsp- |
| | GAGCCCTGGCAGCCCCCCAGACCTGGGCAGGCAGCTGTGAG | SII(SH2211)- |
| | CCAGTCATCACCTACAGCAACATCGGAGTGTGCAAGAACGG | MITD |
| | AGCCCTGGTGTTCATCAACGTGACCCACAGCGACGGAGATG | |
| | TCCAGCCCATCAGCACAGGAAATGTGACCATCCCAACCAACT | |
| | TCACCATCAGCGTCCAGGTGGAATACATGCAGGTGTACACCA | |
| | CCCCAGTGTCCATCGACTGTGCCAGATACGTGTGCAATGGAA | |
| | ACCCCAGATGCAACAAGCTCCTCACCCAGTACGTGTCAGCCT | |
| | GCCAGACAATCGAGCAGGCCCTGGCCATGGGAGCCAGGCTC | |
| | GAGAACATGGAAGTGGACAGCATGCTGTTTGTCTCAGAGAA | |
| | TGCCCTGAAACTGGCCAGCGTGGAGGCCTTCAACAGCACAG | |
| | AGAACCTGGACCCCATCTACAAGGAGTGGCCATCAATCGGA | |
| | GGCAGCTGGCTGGGAGGACTTAAGGACATCCTGCCAAGCCA | |
| | CAACAGCAAAAGAAAGTACGGCAGCGCCATTGAGGACCTGC | |
| | TGTTTGACAAGGTGGTCACCTCCGGCCTGGGCACAGTGGAT | |
| | GAGGACTACAAGAGATGCACCGGCGGCTATGACATTGCCGA | |
| | CCTGGTGTGTGCCCAGTACTACAATGGCATCATGGTGCTGCC | |
| | TGGAGTGGCCAACGCCGACAAAATGACCATGTACACCGCCT | |
| | CCCTGGCTGGAGGCATCACACTGGGAGCCCTGGGGGGAGGA | |
| | GCAGTGGCCATCCCCTTTGCAGTGGCTGTGCAGGCCAGACTC | |
| | AACTACGTGGCCCTGCAGACAGACGTGCTCAACAAGAACCA | |
| | GCAGAACCTGGCCAATGCCTTCATCCAGGCTATCGGAAACAT | |
| | CACCCAGGCCTTTGGAAAAGTGAATGATGCCATCCACCAGAC | |
| | CAGCCAGGGCCTGGCCACAGTGGCCAAGGCCCTGGCCAAGG | |
| | TGCAGGACGTGGTCAACACCCAGGGCCAGGCCCTCAGTCAC | |
| | CTCACAGTACAGCTCCAGAACAACTTCCAGGCAATCTCCTCC | |
| | TCCATCAGCGACATCTACAACAGGCTGGACCCCCCAAGCGCT | |
| | GATGCCCAGGTGGACAGACTGATCACAGGAAGACTCACAGC | |
| | CCTCAACGCATTTGTGTCCCAGACACTGACCAGGCAGGCAG | |
| | AGGTCAGGGCCAGCAGGCAGCTGGCCAAGGACAAGGTGAA | |
| | TGAGTGCGTGAGGAGCCAGAGCCAGAGATTTGGCTTCTGCG | |
| | GAAACGGCACCCACCTGTTCAGCCTGGCCAACGCCGCCCCC | |
| | AACGGCATGATTTTCTTCCACACAGTCCTCCTCCCCACAGCC | |
| | TACGAAACAGTGACAGCCTGGTCAGGCATCTGTGCCAGCGA | |
| | CGGAGACAGAACCTTTGGCCTGGTGGTGAAGGATGTGCAGC | |
| | TCACCCTCTTCAGAAACCTGGATGACAAGTTCTACCTCACCC | |
| | CAAGAACCATGTACCAGCCCAGAGTGGCCACAAGCAGCGAC | |
| | TTTGTGCAGATTGAGGGCTGTGACGTGCTGTTTGTGAATGCA | |
| | ACAGTGATTGACCTCCCAAGCATCATCCCAGATTACATCGAC | |
| | ATCAACCAGACAGTGCAGGACATCCTGGAGAACTACAGGCC | |
| | CAACTGGACAGTGCCAGAGTTCACCCTGGACATCTTCAACG | |
| | CCACCTACCTGAACCTGACAGGAGAAATTGACGACCTGGAG | |
| | TTCAGATCAGAAAAACTTCACAACACCACCGTGGAGCTTGC | |
| | CATCCTCATTGACACCATTAACAACACACTGGTCAACCTGGA | |
| | ATGGCTGAACAGAATTGAAACCTACGTGAAGTGGCCCTGGTA | |
| | TGTGTGGCTGCTGATTGGACTGGTGGTGGTGTTCTGCATCCC | |
| | ACTGCTGCTGTTCTGCTGCTTCAGCACCGGCTGCTGTGGATG | |
| | CATCGGCTGCTTGGGCAGCTGCTGCCACAGCATCTGCAGCAG | |
| | GAGGCAGTTTGAGTACTACGAGCCCATCGAGAAGGTGCACG | |
| | TGCACTTCCTGGGCATCATCGCCGGCGTGGTGGTCCTGGTGG | |
| | TCACAGTGGTGGTGGGAGCTGTGATCTGGAGAAAGAAGTGC | |
| | AGCGGCAGGAAGGGCCCAAGCTACAGCCACGCTGCCAGAG | |
| | ATGACTCCACCCAGGGCAGCGACAGCAGCCTGATGGCCCCC | |
| | AAGGTG | |
| 195 | ATGAGATTTGTGATGTCCCCCACTGTCCTGCTGCTGCTCCTGG | MHCIsp- |
| | GAGCCCTGGCAGCCCCCCAGACCTGGGCAGGCAGCGACCGC | M(2-C11 Re |
| | TACTGTGCCATGCAGCACGCCAGCAGCACCAGCTGCATCAAT | 10276)- |
| | GGCACCAGCACCAACAGCTGCCAGACCTGCTTTGAAAGAGG | MITD |
| | AGACTTGATTTGGCACCTGGCCAACTGGAACTTCAGCTGGA | |
| | GCGTCATCCTCATCGTGTTCATCACCGTGCTGCAGTATGGAAG | |
| | ACCCCAGCTGAGCTGGTTTGTGTACGGCATCAAGATGCTCAT | |
| | CATGTGGCTGCTGTGGCCCATCGTGCTGGCCCTGACCATCTT | |
| | CAACGCCTACAGCGAGTACCAGGTGTCCAGATACGTGATGTT | |
| | TGGCTTCTCTGTGGCTGGAGCTGTCATCACCTTTGCCCTGTG | |
| | GATGATGTACTTTGTGAGGAGCATCCAGCTGTACAGGAGGAC | |
| | CAAGAGCTGGTGGAGCTTCAACCCAGAAACCAATGCCATCC | |
| | TGTGTGTGAATGCCCTGGGCAGGAGCTACGTGCTGCCCCTGG | |
| | ATGGCATCCCCACTGGAGTCACCCTGACCCTGCTGTCTGGAA | |
| | ACCTGTATGCTGAGTGCTTCAAGATGGTGGGCGGCCTGACCA | |
| | TCGAGCACCTGCCCAAGTACGTGATGATTGCCACCCCCAGCA | |
| | GCACCATCGTGTACACCCTGGTGGGCAAGCAGCTGAAGGCC | |
| | ACCACAGCCACCGGCTGGGCCTACTATGTGAAGAGCAAGGC | |
| | TGGAGACTACAGCACAGAGGCCAGGACAGACAACCTGTCTG | |
| | AACATGAGAAGCTGCTGCACATGGTGTTCCTGGGCATCATCG | |
| | CCGGCGTGGTGGTCCTGGTGGTCACAGTGGTGGTGGGAGCT | |
| | GTGATCTGGAGAAAGAAGTGCAGCGGCAGGAAGGGCCCAA | |
| | GCTACAGCCACGCTGCCAGAGATGACTCCACCCAGGGCAGC | |
| | GACAGCAGCCTGATGGCCCCCAAGGTG | |
| 196 | ATGGCCACCCAGGGCCAGAGGGTCAACTGGGGCGACGAGCC | N(2-C11 Re |
| | CAGCAAGAGGAGAGGAAGAAGCAACAGCAGAGGAAGGAA | 10276) |
| | GAACAATGACATCCCCCTGAGCTTCTACAACCCCATCACCCT | |
| | GGAGACTGGCAGCAAGTTCTGGAATGTCTGCCCCAGGGACT | |
| | TTGTGCCCAAGGGCATCGGCAACAAGGACCAGCAGATTGGC | |
| | TACTGGAACAAGCAGGCCCGCTACAGAATTGTGAAGGGCCA | |
| | GAGGAAGGACCTGCCTGAGAGGTGGTTCTTCTACTTCCTGGG | |
| | CACAGGCCCCCACGCTGATGCCAAGTTCAAGGACAAGATTG | |
| | ATGGAGTCTTCTGGGTGGCCAAGGATGGAGCCATGAACAAG | |
| | CCCACCACCCTGGGCACCAGAGGCACCAACAATGAGAGCAA | |
| | GCCCCTGAGATTTGATGGGAAGATCCCCCCCCAGTTCCAGCT | |
| | GGAGGTGAACCAGAGCAGAAACAACAGCAGAAGCGGCAGC | |
| | CAGAGCAGAAGTGCCTCCAGAAACAGAAGCCAGAGCAGAG | |
| | GAAGACAGCAGAGCAACAACCAGAACACCAACGTGGAGGA | |
| | CACCATCGTGGCTGTGCTGCAGAAGCTGGGCGTGACAGACA | |
| | AGCAGAGGAGCAGAAGCAAGAGCAGAGAAAGAAGCGGCA | |
| | GCAACAGCAGGGACACCACCCCCAAGAATGCCAACAAGCAC | |
| | AGCTGGAAGAAGACAGCTGGCAAGGGAGATGTGACCAACTT | |
| | CTATGGAGCCAGGAGTGCCAGCGCCAACTTCGGGGACAGTG | |
| | ACCTGGTGGCCAATGGAAATGCCGCCAAGTGCTACCCCCAG | |
| | ATTGCTGAGTGTGTGCCCAGCGTGTCCTCCATGCTGTTTGGA | |
| | AGCCAGTGGTCAGCAGAGGATGCTGGGGACCAGGTGAAGGT | |
| | GACCCTGACCCACACCTACTACCTGCCCAAGGATGATGCCAA | |
| | GACCAGCCAGTTCCTGGGCCAGATTGATGCCTACAAGAGGCC | |
| | CAGCCAGGTGGTGAAGGAGCAGAGGCAGAGGAAGAGCAGA | |
| | AGCAAGTCTGCTGACAAGAAGCCAGAGGAGCTGTCTGTGAC | |
| | CCTGGTGGAGGCCTACACAGACGTGTTTGATGACACCCAGGT | |
| | GGAGATGATTGATGAGGTGACCAAC | |
| 197 | ATGAGATTTGTGATGTCCCCCACTGTCCTGCTGCTGCTCCTGG | MHCIsp- |
| | GAGCCCTGGCAGCCCCCCAGACCTGGGCAGGCAGCTGTGAG | SII(2-C11 |
| | CCCATCATCACCTACTTCAACATCGGGGTGTGCAAGAATGGG | Re 10276)- |
| | GCCCTGGTCTTCATCAATGTGACCCACAGCGATGGAGATGTG | MITD |
| | CAGCCCATCAGCACAGGAAATGTGACCATCCCCACCAACTTC | |
| | ACCATCTCTGTGCAGGTGGAGTACATCCAGGTGTACACCACC | |
| | CCTGTGTCCATCGACTGCAGCCGCTACGTGTGCAATGGAAAC | |
| | CCCAGGTGCAACAAGCTGCTGACCCAGTACTTCTCTGCCTGC | |
| | CAGACCATCGAGCAGGCCCTGGCCATGGGAGCCCGGCTGGA | |
| | GAACATGGAGGTGGACAGCATGCTGTTTGTGTCTGAAAATGC | |
| | CCTGAAGCTGGCCTCTGTGGAGGCCTTCAACAGCAGTGAGC | |
| | ACCTGGACCCCATCTACAAGGAGTGGCCCAACATTGGAGGC | |
| | AGCTGGCTGGGGGGCCTGAAGGACATCCTGCCCAGCCACAA | |
| | CAGCAAGAGGAACTACAGAAGTGCCATCGAGGACCTGCTCT | |
| | TTGACAAGGTGGTGACCTCTGGGCTGGGCACCGTGGATGAT | |
| | GACTACAAGAGGTGCACAGGAGGCTATGACATTGCAGACCT | |
| | GGTGTGTGCCCAGTACTACCATGGCATCATGGTGCTGCCTGG | |
| | AGTGGCCAATGATGACAAGATGACCATGTACACAGCCTCCCT | |
| | GGCTGGAGGCATCACCCTGGGGGCCCTGGGCGGAGGAGCCG | |
| | TGGCCATCCCCTTTGCTGTGGCTGTGCAGGCCAGGCTCAACT | |
| | ACGTGGCCCTGCAGACAGATGTGCTCAACAAGAACCAGCAG | |
| | ATCCTGGCCAACGCCTTCAACCAGGCCATTGGAAACATCACC | |
| | CAGGCCTTTGGGAAGGTGAATGATGCCATCCACCAGACCAG | |
| | CAAGGGCCTGGCCACCGTGGCCAAGGCCCTGGCCAAGGTGC | |
| | AGGATGTGGTCAACACCCAGGGCCAGGCCCTGAGCCACCTC | |
| | ACTGTCCAGCTGCAGAACAACTTCCAGGCCATCAGCAGCAG | |
| | CATCTCTGACATCTACAACAGGCTGGATGAGCTGTCTGCTGA | |
| | TGCCCAGGTGGACAGACTCATCACCGGGAGGCTGACAGCCC | |
| | TGAATGCCTTTGTCAGCCAGACCCTGACCAGGCAGGCAGAG | |
| | GTGCGGGCCTCCCGGCAGCTGGCCAAGGACAAGGTGAATGA | |
| | GTGTGTGAGGAGCCAGAGCCAGAGGTTTGGCTTCTGTGGAA | |
| | ATGGCACCCACCTCTTCTCCCTGGCCAATGCTGCCCCCAATG | |
| | GCATGATCTTCTTCCACACCGTGCTGCTGCCCACCGCCTATGA | |
| | AACAGTGACAGCCTGGAGCGGCATCTGTGCCTCTGATGGGG | |
| | ACCACACCTTCGGCCTGGTGGTGAAGGATGTCCAGCTGACC | |
| | CTCTTCAGAAACCTGGATGACAAGTTCTACCTGACCCCCAGG | |
| | ACCATGTACCAGCCCCGGGTGGCCACCAGCAGCGACTTTGTG | |
| | CAGATTGAGGGCTGTGATGCCCTGTTTGTGAATGCCACTGTC | |
| | ATCGAGCTGCCCAGCATCATCCCAGACTACATTGACATCAAC | |
| | CAGACCGTGCAGGACATCCTGAAGAACTACAGGCCCAACTG | |
| | GACAGTTCCTGAGCTGACCCTGGACATCTTCAACAGCACCTA | |
| | CCTGAACCTGACAGGAGAAATCAATGACCTGGAGTTCAGAA | |
| | GTGAGAAGCTGCACAACACCACAGTGGAGCTGGCTGTGCTG | |
| | ATCGACAACATCAACAACACCCTGGTCAACCTGGAGTGGCT | |
| | GAACAGAATTGAAACCTACGTGAAGTGGCCCTGGTATGTTTG | |
| | GCTGCTCATCGGCCTGGTGCTGGTGTTCTGCATCCCCCTGCTC | |
| | ATGTTCTGCTGCCTGAGCACCGGCTGCTGCGGCTGCTTCGGC | |
| | TGCCTGGGCAGCTGCTGCCACAGCCTGTTCTCCAGAAGACA | |
| | CTTTGAGAACTACGAGCCCATCGAGAAGGTGCACATCCACTT | |
| | CCTGGGCATCATCGCCGGCGTGGTGGTCCTGGTGGTCACAGT | |
| | GGTGGTGGGAGCTGTGATCTGGAGAAAGAAGTGCAGCGGCA | |
| | GGAAGGGCCCAAGCTACAGCCACGCTGCCAGAGATGACTCC | |
| | ACCCAGGGCAGCGACAGCAGCCTGATGGCCCCCAAGGTG | |
| 198 | ATGAGATTCGTGATGTCCCCTACCGTACTACTACTCCTACTTG | MHCIsp- |
| | GCGCACTAGCAGCTCCTCAAACTTGGGCCGGATCCTACCCAT | HA-M- |
| | ACGATGTTCCAGACTACGCTTACCCATATGACGTGCCAGACT | MITD-2A- |
| | ATGCCTACCCCTACGACGTGCCCGACTACGCAGAGAGGTACT | His-N |
| | GTGCCATGCAGAACACCGGTTCCCAGTGCATCAACGGCACA | |
| | GACTCCTCCTGCTCCACCTGCTTCGAGAGAGGAGGCCTGATC | |
| | TGGCACCTGGCAAACTGGAACTTCAGCTGGAGCGTGATCCT | |
| | GATAGTGTTCATAACCGTCCTGAAGTACGGCAGACCACAGTT | |
| | CTCATGGCTTGTCTATGGCATCAAGATGCTGATTATGTGGCTG | |
| | CTTTGGCCTATCGTCCTGGCCCTGACCATCTTCAACGCCTACT | |
| | CTGAGTACCAGGTGTCAAGGTATGTCATGTTCGGCTTCTCAG | |
| | TGGCTGGAGCTGTGGTGACCTTTGCTCTGTGGATGATGTACT | |
| | TCGTGAGGTCCATCCAGCTGTACAGGAGGACAAAGTCATGGT | |
| | GGTCCTTCAACCCAGAAACCAATGCCATCCTGTGCGTCAACG | |
| | CACTGGGCAGAAGCTACGTCCTACCACTGGACGGCACTCCTA | |
| | CAGGAGTGACCCTGACCCTGCTGTCAGGCAATCTGTACGCAG | |
| | AGGGGTTCAAGATGGCCGGTGGCCTGACCATCGAGCATCTGC | |
| | CTAAGTACGTGATGATCGCCACCCCTAGCAGGACAATCGTGT | |
| | ACACCCTGGTGGGAAAGCAGCTAAAGGCGACCACAGCCACA | |
| | GGCTGGGCCTACTACGTGAAGTCCAAGGCAGGGGACTATTC | |
| | AACCGAGGCCAGGACCGACAACCTGTCAGAGCACGAGAAG | |
| | CTGCTGCACATGGTCTTCCTGGGCATCATCGCAGGAGTGGTG | |
| | GTGCTGGTGGTGACCGTGGTGGTGGGGGCTGTAATCTGGAG | |
| | GAAGAAGTGCTCAGGGAGAAAGGGCCCAAGCTACTCTCACG | |
| | CCGCCAGGGATGACTCCACACAGGGCTCAGACTCCTCACTG | |
| | ATGGCTCCAAAGGTCAGAGCCAAGAGAGGCAGCGGAGCCA | |
| | CCAACTTCAGCCTGCTGAAGCAGGCCGGCGACGTGGAGGAG | |
| | AACCCAGGACCTCACCACCACCACCACCACGCCACACAGGG | |
| | CCAGAGGGTGAACTGGGGCGACGAGCCATCCAAGAGGAGG | |
| | GGAAGGAGCAACAGCAGAGGAAGGAAGAACAACACCATCC | |
| | CCCTGTCCTTCTTCAACCCAATTCAGCTAGAGCCAGGCAGCA | |
| | AGTTCTGGTCAGTGTGCCCCAGAGACTTCGTGCCCAAGGGC | |
| | ATCGGAAACAAGGACCAGCAGATCGGCTACTGGAACAGACA | |
| | GGAGAGATACAGAATTGTGAAAGGCCAGAGAAAGGAGCTGC | |
| | CAGAGAGGTGGTTCTTCTACTTCCTGGGCACCGGCCCACAGG | |
| | CAGACGCCAAGTTCAAGGACAAGATCGATGGAGTGTTCTGG | |
| | GTGGCCAAGGACGGCGCCATGAACAAGCCCACCACACTGGG | |
| | CACAAGAGGAACAAACAATGAGAGCAAGCCACTGAAGTTTG | |
| | ATGGCAAGATCCCACCCCAGTTCCAGCTGGAGGTGAACAGG | |
| | AGCAGAAACAACAGCAGAAGCGGCAGCCAGAGCAGAAGTG | |
| | TGAGCAGAAACAGAAGCCAGAGCAGAGGAAGACAGCAGAG | |
| | CAACAACCAGAACAACGTGGAGGACACCATCGTGGCCGTGC | |
| | TGCAGAAGCTGGGGGTCACAGAAAAGCAGAGGAGCAGAAG | |
| | CAAGAGCAGGGACAGAGGAGACAGCAAGCCAAGAGACACC | |
| | ACCCCCAACAACGCCAACAAGCACACCTGGAAGAAGACAG | |
| | CCGGCAAGGGAGATGTGACCAACTTCTACGGCGCCAGAAGC | |
| | GCCAGCGCCAACTTCGGAGACTCAGACCTGGTGGCCAATGG | |
| | AAACGCAGCCAAGAGCTACCCCCAGATCGCAGAGTGTGTGC | |
| | CCTCTGTCTCCAGCATGCTGTTTGGCAGCCAGTGGAGCGCCG | |
| | AGGACGACGGTGACCAGGTGAAGGTGACCCTGACACACAC | |
| | ATACTACCTGCCCAAAGATGACGCCAAGACCAGCCAGTTCCT | |
| | GGAGCAGATTGATGCCTACAAGAGGCCCAGCCAGGTGGCCA | |
| | AGGACCAGAGACAGAGGAAGAGCAGGTCCAAGAGCGCCGA | |
| | GAAGAAGCCAGAAGAATTGAGTGTCACCCTGGTGGAGGCCT | |
| | ACACAGACGTGTTTGATGACACCCAGGTGGAGATGATTGATG | |
| | AGGTGACCAAC | |
| 199 | ATGAGATTCGTGATGTCCCCTACCGTACTACTACTCCTACTTG | MHCIsp- |
| | GCGCACTAGCAGCTCCTCAAACTTGGGCCGGATCCTACCCAT | HA-M-GS- |
| | ACGATGTTCCAGACTACGCTTACCCATATGACGTGCCAGACT | N-MITD |
| | ATGCCTACCCCTACGACGTGCCCGACTACGCAGAGAGGTACT | |
| | GTGCCATGCAGAACACCGGTTCCCAGTGCATCAACGGCACA | |
| | GACTCCTCCTGCTCCACCTGCTTCGAGAGAGGAGGCCTGATC | |
| | TGGCACCTGGCAAACTGGAACTTCAGCTGGAGCGTGATCCT | |
| | GATAGTGTTCATAACCGTCCTGAAGTACGGCAGACCACAGTT | |
| | CTCATGGCTTGTCTATGGCATCAAGATGCTGATTATGTGGCTG | |
| | CTTTGGCCTATCGTCCTGGCCCTGACCATCTTCAACGCCTACT | |
| | CTGAGTACCAGGTGTCAAGGTATGTCATGTTCGGCTTCTCAG | |
| | TGGCTGGAGCTGTGGTGACCTTTGCTCTGTGGATGATGTACT | |
| | TCGTGAGGTCCATCCAGCTGTACAGGAGGACAAAGTCATGGT | |
| | GGTCCTTCAACCCAGAAACCAATGCCATCCTGTGCGTCAACG | |
| | CACTGGGCAGAAGCTACGTCCTACCACTGGACGGCACTCCTA | |
| | CAGGAGTGACCCTGACCCTGCTGTCAGGCAATCTGTACGCAG | |
| | AGGGGTTCAAGATGGCCGGTGGCCTGACCATCGAGCATCTGC | |
| | CTAAGTACGTGATGATCGCCACCCCTAGCAGGACAATCGTGT | |
| | ACACCCTGGTGGGAAAGCAGCTAAAGGCGACCACAGCCACA | |
| | GGCTGGGCCTACTACGTGAAGTCCAAGGCAGGGGACTATTC | |
| | AACCGAGGCCAGGACCGACAACCTGTCAGAGCACGAGAAG | |
| | CTGCTGCACATGGTCGGAGGAGGAGGAAGCGGAGGAGGAG | |
| | GAAGCGGAGGAGGAGGAAGCGCCACACAGGGCCAGAGGGT | |
| | GAACTGGGGCGACGAGCCATCCAAGAGGAGGGGAAGGAGC | |
| | AACAGCAGAGGAAGGAAGAACAACACCATCCCCCTGTCCTT | |
| | CTTCAACCCAATTCAGCTAGAGCCAGGCAGCAAGTTCTGGTC | |
| | AGTGTGCCCCAGAGACTTCGTGCCCAAGGGCATCGGAAACA | |
| | AGGACCAGCAGATCGGCTACTGGAACAGACAGGAGAGATAC | |
| | AGAATTGTGAAAGGCCAGAGAAAGGAGCTGCCAGAGAGGT | |
| | GGTTCTTCTACTTCCTGGGCACCGGCCCACAGGCAGACGCCA | |
| | AGTTCAAGGACAAGATCGATGGAGTGTTCTGGGTGGCCAAG | |
| | GACGGCGCCATGAACAAGCCCACCACACTGGGCACAAGAGG | |
| | AACAAACAATGAGAGCAAGCCACTGAAGTTTGATGGCAAGA | |
| | TCCCACCCCAGTTCCAGCTGGAGGTGAACAGGAGCAGAAAC | |
| | AACAGCAGAAGCGGCAGCCAGAGCAGAAGTGTGAGCAGAA | |
| | ACAGAAGCCAGAGCAGAGGAAGACAGCAGAGCAACAACCA | |
| | GAACAACGTGGAGGACACCATCGTGGCCGTGCTGCAGAAGC | |
| | TGGGGGTCACAGAAAAGCAGAGGAGCAGAAGCAAGAGCAG | |
| | GGACAGAGGAGACAGCAAGCCAAGAGACACCACCCCCAAC | |
| | AACGCCAACAAGCACACCTGGAAGAAGACAGCCGGCAAGG | |
| | GAGATGTGACCAACTTCTACGGCGCCAGAAGCGCCAGCGCC | |
| | AACTTCGGAGACTCAGACCTGGTGGCCAATGGAAACGCAGC | |
| | CAAGAGCTACCCCCAGATCGCAGAGTGTGTGCCCTCTGTCTC | |
| | CAGCATGCTGTTTGGCAGCCAGTGGAGCGCCGAGGACGACG | |
| | GTGACCAGGTGAAGGTGACCCTGACACACACATACTACCTG | |
| | CCCAAAGATGACGCCAAGACCAGCCAGTTCCTGGAGCAGAT | |
| | TGATGCCTACAAGAGGCCCAGCCAGGTGGCCAAGGACCAGA | |
| | GACAGAGGAAGAGCAGGTCCAAGAGCGCCGAGAAGAAGCC | |
| | AGAAGAATTGAGTGTCACCCTGGTGGAGGCCTACACAGACG | |
| | TGTTTGATGACACCCAGGTGGAGATGATTGATGAGGTGACCA | |
| | ACTTCCTGGGCATCATCGCAGGAGTGGTGGTGCTGGTGGTGA | |
| | CCGTGGTGGTGGGGGCTGTAATCTGGAGGAAGAAGTGCTCA | |
| | GGGAGAAAGGGCCCAAGCTACTCTCACGCCGCCAGGGATGA | |
| | CTCCACACAGGGCTCAGACTCCTCACTGATGGCTCCAAAGGT | |
| | C | |
| 200 | ATGAGATTCGTGATGTCCCCTACCGTACTACTACTCCTACTTG | MHCIsp-M- |
| | GCGCACTAGCAGCTCCTCAAACTTGGGCCGGATCCGAGAGG | MITD-2A-N |
| | TACTGTGCCATGCAGAACACCGGTTCCCAGTGCATCAACGGC | |
| | ACAGACTCCTCCTGCTCCACCTGCTTCGAGAGAGGAGGCCT | |
| | GATCTGGCACCTGGCAAACTGGAACTTCAGCTGGAGCGTGA | |
| | TCCTGATAGTGTTCATAACCGTCCTGAAGTACGGCAGACCAC | |
| | AGTTCTCATGGCTTGTCTATGGCATCAAGATGCTGATTATGTG | |
| | GCTGCTTTGGCCTATCGTCCTGGCCCTGACCATCTTCAACGCC | |
| | TACTCTGAGTACCAGGTGTCAAGGTATGTCATGTTCGGCTTCT | |
| | CAGTGGCTGGAGCTGTGGTGACCTTTGCTCTGTGGATGATGT | |
| | ACTTCGTGAGGTCCATCCAGCTGTACAGGAGGACAAAGTCAT | |
| | GGTGGTCCTTCAACCCAGAAACCAATGCCATCCTGTGCGTCA | |
| | ACGCACTGGGCAGAAGCTACGTCCTACCACTGGACGGCACT | |
| | CCTACAGGAGTGACCCTGACCCTGCTGTCAGGCAATCTGTAC | |
| | GCAGAGGGGTTCAAGATGGCCGGTGGCCTGACCATCGAGCA | |
| | TCTGCCTAAGTACGTGATGATCGCCACCCCTAGCAGGACAAT | |
| | CGTGTACACCCTGGTGGGAAAGCAGCTAAAGGCGACCACAG | |
| | CCACAGGCTGGGCCTACTACGTGAAGTCCAAGGCAGGGGAC | |
| | TATTCAACCGAGGCCAGGACCGACAACCTGTCAGAGCACGA | |
| | GAAGCTGCTGCACATGGTCTTCCTGGGCATCATCGCAGGAGT | |
| | GGTGGTGCTGGTGGTGACCGTGGTGGTGGGGGCTGTAATCTG | |
| | GAGGAAGAAGTGCTCAGGGAGAAAGGGCCCAAGCTACTCTC | |
| | ACGCCGCCAGGGATGACTCCACACAGGGCTCAGACTCCTCA | |
| | CTGATGGCTCCAAAGGTCAGAGCCAAGAGAGGCAGCGGAGC | |
| | CACCAACTTCAGCCTGCTGAAGCAGGCCGGCGACGTGGAGG | |
| | AGAACCCAGGACCTGCCACACAGGGCCAGAGGGTGAACTG | |
| | GGGCGACGAGCCATCCAAGAGGAGGGGAAGGAGCAACAGC | |
| | AGAGGAAGGAAGAACAACACCATCCCCCTGTCCTTCTTCAA | |
| | CCCAATTCAGCTAGAGCCAGGCAGCAAGTTCTGGTCAGTGTG | |
| | CCCCAGAGACTTCGTGCCCAAGGGCATCGGAAACAAGGACC | |
| | AGCAGATCGGCTACTGGAACAGACAGGAGAGATACAGAATT | |
| | GTGAAAGGCCAGAGAAAGGAGCTGCCAGAGAGGTGGTTCTT | |
| | CTACTTCCTGGGCACCGGCCCACAGGCAGACGCCAAGTTCA | |
| | AGGACAAGATCGATGGAGTGTTCTGGGTGGCCAAGGACGGC | |
| | GCCATGAACAAGCCCACCACACTGGGCACAAGAGGAACAA | |
| | ACAATGAGAGCAAGCCACTGAAGTTTGATGGCAAGATCCCA | |
| | CCCCAGTTCCAGCTGGAGGTGAACAGGAGCAGAAACAACA | |
| | GCAGAAGCGGCAGCCAGAGCAGAAGTGTGAGCAGAAACAG | |
| | AAGCCAGAGCAGAGGAAGACAGCAGAGCAACAACCAGAAC | |
| | AACGTGGAGGACACCATCGTGGCCGTGCTGCAGAAGCTGGG | |
| | GGTCACAGAAAAGCAGAGGAGCAGAAGCAAGAGCAGGGAC | |
| | AGAGGAGACAGCAAGCCAAGAGACACCACCCCCAACAACG | |
| | CCAACAAGCACACCTGGAAGAAGACAGCCGGCAAGGGAGA | |
| | TGTGACCAACTTCTACGGCGCCAGAAGCGCCAGCGCCAACT | |
| | TCGGAGACTCAGACCTGGTGGCCAATGGAAACGCAGCCAAG | |
| | AGCTACCCCCAGATCGCAGAGTGTGTGCCCTCTGTCTCCAGC | |
| | ATGCTGTTTGGCAGCCAGTGGAGCGCCGAGGACGACGGTGA | |
| | CCAGGTGAAGGTGACCCTGACACACACATACTACCTGCCCA | |
| | AAGATGACGCCAAGACCAGCCAGTTCCTGGAGCAGATTGAT | |
| | GCCTACAAGAGGCCCAGCCAGGTGGCCAAGGACCAGAGAC | |
| | AGAGGAAGAGCAGGTCCAAGAGCGCCGAGAAGAAGCCAGA | |
| | AGAATTGAGTGTCACCCTGGTGGAGGCCTACACAGACGTGTT | |
| | TGATGACACCCAGGTGGAGATGATTGATGAGGTGACCAAC | |
| 201 | ATGAGATTCGTGATGTCCCCTACCGTACTACTACTCCTACTTG | MHCIsp-M- |
| | GCGCACTAGCAGCTCCTCAAACTTGGGCCGGATCCGAGAGG | GS-N- |
| | TACTGTGCCATGCAGAACACCGGTTCCCAGTGCATCAACGGC | MITD |
| | ACAGACTCCTCCTGCTCCACCTGCTTCGAGAGAGGAGGCCT | |
| | GATCTGGCACCTGGCAAACTGGAACTTCAGCTGGAGCGTGA | |
| | TCCTGATAGTGTTCATAACCGTCCTGAAGTACGGCAGACCAC | |
| | AGTTCTCATGGCTTGTCTATGGCATCAAGATGCTGATTATGTG | |
| | GCTGCTTTGGCCTATCGTCCTGGCCCTGACCATCTTCAACGCC | |
| | TACTCTGAGTACCAGGTGTCAAGGTATGTCATGTTCGGCTTCT | |
| | CAGTGGCTGGAGCTGTGGTGACCTTTGCTCTGTGGATGATGT | |
| | ACTTCGTGAGGTCCATCCAGCTGTACAGGAGGACAAAGTCAT | |
| | GGTGGTCCTTCAACCCAGAAACCAATGCCATCCTGTGCGTCA | |
| | ACGCACTGGGCAGAAGCTACGTCCTACCACTGGACGGCACT | |
| | CCTACAGGAGTGACCCTGACCCTGCTGTCAGGCAATCTGTAC | |
| | GCAGAGGGGTTCAAGATGGCCGGTGGCCTGACCATCGAGCA | |
| | TCTGCCTAAGTACGTGATGATCGCCACCCCTAGCAGGACAAT | |
| | CGTGTACACCCTGGTGGGAAAGCAGCTAAAGGCGACCACAG | |
| | CCACAGGCTGGGCCTACTACGTGAAGTCCAAGGCAGGGGAC | |
| | TATTCAACCGAGGCCAGGACCGACAACCTGTCAGAGCACGA | |
| | GAAGCTGCTGCACATGGTCGGAGGAGGAGGAAGCGGAGGA | |
| | GGAGGAAGCGGAGGAGGAGGAAGCGCCACACAGGGCCAGA | |
| | GGGTGAACTGGGGCGACGAGCCATCCAAGAGGAGGGGAAG | |
| | GAGCAACAGCAGAGGAAGGAAGAACAACACCATCCCCCTGT | |
| | CCTTCTTCAACCCAATTCAGCTAGAGCCAGGCAGCAAGTTCT | |
| | GGTCAGTGTGCCCCAGAGACTTCGTGCCCAAGGGCATCGGA | |
| | AACAAGGACCAGCAGATCGGCTACTGGAACAGACAGGAGA | |
| | GATACAGAATTGTGAAAGGCCAGAGAAAGGAGCTGCCAGAG | |
| | AGGTGGTTCTTCTACTTCCTGGGCACCGGCCCACAGGCAGAC | |
| | GCCAAGTTCAAGGACAAGATCGATGGAGTGTTCTGGGTGGC | |
| | CAAGGACGGCGCCATGAACAAGCCCACCACACTGGGCACAA | |
| | GAGGAACAAACAATGAGAGCAAGCCACTGAAGTTTGATGGC | |
| | AAGATCCCACCCCAGTTCCAGCTGGAGGTGAACAGGAGCAG | |
| | AAACAACAGCAGAAGCGGCAGCCAGAGCAGAAGTGTGAGC | |
| | AGAAACAGAAGCCAGAGCAGAGGAAGACAGCAGAGCAACA | |
| | ACCAGAACAACGTGGAGGACACCATCGTGGCCGTGCTGCAG | |
| | AAGCTGGGGGTCACAGAAAAGCAGAGGAGCAGAAGCAAGA | |
| | GCAGGGACAGAGGAGACAGCAAGCCAAGAGACACCACCCC | |
| | CAACAACGCCAACAAGCACACCTGGAAGAAGACAGCCGGC | |
| | AAGGGAGATGTGACCAACTTCTACGGCGCCAGAAGCGCCAG | |
| | CGCCAACTTCGGAGACTCAGACCTGGTGGCCAATGGAAACG | |
| | CAGCCAAGAGCTACCCCCAGATCGCAGAGTGTGTGCCCTCTG | |
| | TCTCCAGCATGCTGTTTGGCAGCCAGTGGAGCGCCGAGGAC | |
| | GACGGTGACCAGGTGAAGGTGACCCTGACACACACATACTA | |
| | CCTGCCCAAAGATGACGCCAAGACCAGCCAGTTCCTGGAGC | |
| | AGATTGATGCCTACAAGAGGCCCAGCCAGGTGGCCAAGGAC | |
| | CAGAGACAGAGGAAGAGCAGGTCCAAGAGCGCCGAGAAGA | |
| | AGCCAGAAGAATTGAGTGTCACCCTGGTGGAGGCCTACACA | |
| | GACGTGTTTGATGACACCCAGGTGGAGATGATTGATGAGGTG | |
| | ACCAACTTCCTGGGCATCATCGCAGGAGTGGTGGTGCTGGTG | |
| | GTGACCGTGGTGGTGGGGGCTGTAATCTGGAGGAAGAAGTG | |
| | CTCAGGGAGAAAGGGCCCAAGCTACTCTCACGCCGCCAGGG | |
| | ATGACTCCACACAGGGCTCAGACTCCTCACTGATGGCTCCAA | |
| | AGGTC | |

TABLE 4
Composition of single antigen sequences involved
in the embodiments of the present disclosure

| | | SEQ ID NO: | | | |
|---|---|---|---|---|---|
| | | Signal | | | |
| | | peptide/ | | | |
| SEQ | | Start | Tag | | |
| ID NO: | Description | codon | peptide | Gene | MITD |
| 74 | HA-M(M2) | 50 | 34 | 5 | — |
| 75 | HA-M(M3) | 50 | 35 | 6 | — |
| 76 | HA-M(M6) | 50 | 36 | 7 | — |
| 77 | HA-M_d(M2) | 50 | 34 | 51 | — |
| 78 | HA-M_d(M3) | 50 | 35 | 52 | — |
| 79 | HA-M_d(M6) | 50 | 36 | 53 | — |
| 80 | MHCIsp-HA-M(M2) | 17 | 34 | 5 | — |
| 81 | MHCIsp-HA-M(M3) | 18 | 35 | 6 | — |
| 82 | MHCIsp-HA-M(M6) | 20 | 36 | 7 | — |
| 83 | MHCIsp-HA-M(M2)- | 17 | 34 | 5 | 26 |
| | MITD | | | | |
| 84 | MHCIsp-HA-M(M3)- | 18 | 35 | 6 | 25 |
| | MITD | | | | |
| 85 | MHCIsp-HA-M(M6)- | 20 | 36 | 7 | 24 |
| | MITD | | | | |
| 86 | His-N(N2) | 50 | 38 | 9 | — |
| 87 | His-N(N3) | 50 | 38 | 10 | — |
| 88 | His-N(N6) | 50 | 39 | 11 | — |
| 89 | His-N_d(N2) | 50 | 38 | 54 | — |
| 90 | His-N_d(N3) | 50 | 38 | 55 | — |
| 91 | His-N_d(N6) | 50 | 39 | 56 | — |
| 92 | MHCIsp-His-N(N2) | 17 | 38 | 9 | — |
| 93 | MHCIsp-His-N(N3) | 18 | 38 | 10 | — |
| 94 | MHCIsp-His-N(N6) | 19 | 39 | 11 | — |
| 95 | MHCIsp-His-N(N2)- | 17 | 38 | 9 | 27 |
| | MITD | | | | |
| 96 | MHCIsp-His-N(N3)- | 18 | 38 | 10 | 28 |
| | MITD | | | | |
| 97 | MHCIsp-His-N(N6)- | 19 | 39 | 11 | 29 |
| | MITD | | | | |
| 98 | Flag-S(S2) | 50 | 42 | 13 | — |
| 99 | Flag-S(S3) | 50 | 41 | 14 | — |
| 100 | Flag-S(S6) | 50 | 43 | 15 | — |
| 101 | Flag-S_ec(S2) | 50 | 42 | 57 | — |
| 102 | Flag-S_ec(S3) | 50 | 41 | 58 | — |
| 103 | Flag-S_ec(S6) | 50 | 43 | 59 | — |
| 104 | Flag-SII(S2) | 50 | 42 | 60 | — |
| 105 | Flag-SII(S3) | 50 | 41 | 61 | — |
| 106 | Flag-SII(S6) | 50 | 43 | 62 | — |
| 107 | MHCIsp-Flag-S(S2) | 17 | 42 | 13 | — |
| 108 | MHCIsp-Flag-S(S3) | 21 | 41 | 14 | — |
| 109 | MHCIsp-Flag-S(S6) | 22 | 43 | 15 | — |
| 110 | MHCIsp-Flag-S_ec(S2) | 17 | 42 | 57 | — |
| 111 | MHCIsp-Flag-S_ec(S3) | 21 | 41 | 58 | — |
| 112 | MHCIsp-Flag-S_ec(S6) | 22 | 43 | 59 | — |
| 113 | MHCIsp-Flag-SII(S2) | 17 | 42 | 60 | — |
| 114 | MHCIsp-Flag-SII(S3) | 21 | 41 | 61 | — |
| 115 | MHCIsp-Flag-SII(S6) | 22 | 43 | 62 | — |
| 116 | MHCIsp-Flag-S(S2)- | 17 | 42 | 13 | 30 |
| | MITD | | | | |
| 117 | MHCIsp-Flag-S(S3)- | 21 | 41 | 14 | 31 |
| | MITD | | | | |
| 118 | MHCIsp-Flag-S(S6)- | 22 | 43 | 15 | 32 |
| | MITD | | | | |
| 119 | MHCIsp-Flag- | 17 | 42 | 57 | 30 |
| | S_ec(S2)-MITD | | | | |
| 120 | MHCIsp-Flag- | 21 | 41 | 58 | 31 |
| | S_ec(S3)-MITD | | | | |
| 121 | MHCIsp-Flag- | 22 | 43 | 59 | 32 |
| | S_ec(S6)-MITD | | | | |
| 122 | MHCIsp-Flag- | 17 | 42 | 60 | 30 |
| | SII(S2)-MITD | | | | |
| 123 | MHCIsp-Flag- | 21 | 41 | 61 | 31 |
| | SII(S3)-MITD | | | | |
| 124 | MHCIsp-Flag- | 22 | 43 | 62 | 32 |
| | SII(S6)-MITD | | | | |
| 125 | His-7a | 50 | 38 | 72 | — |
| 126 | Flag-3a | 50 | 42 | 73 | — |
| 127 | MHCIsp-HA- | 17 | 36 | 63 | 30 |
| | M(HF1902)-MITD | | | | |
| 128 | His-N(HF1902) | 50 | 38 | 64 | — |
| 129 | MHCIsp-Flag- | 17 | 42 | 65 | 30 |
| | SII(HF1902)-MITD | | | | |
| 130 | MHCIsp-HA- | 17 | 36 | 66 | 30 |
| | M(SH2211)-MITD | | | | |
| 131 | His-N(SH2211) | 50 | 38 | 67 | — |
| 132 | MHCIsp-Flag- | 17 | 42 | 68 | 30 |
| | SII(SH2211)-MITD | | | | |
| 133 | MHCIsp-HA-M(2- | 17 | 36 | 69 | 30 |
| | C11 Re 10276)-MITD | | | | |
| 134 | His-N(2-C11 Re 10276) | 50 | 38 | 70 | — |
| 135 | MHCIsp-Flag-SII(2- | 17 | 42 | 71 | 30 |
| | C11 Re 10276)-MITD | | | | |
| 136 | M(M2) | 50 | — | 5 | — |
| 137 | M(M3) | 50 | — | 6 | — |
| 138 | M(M6) | 50 | — | 7 | — |
| 139 | M_d(M2) | 50 | — | 51 | — |
| 140 | M_d(M3) | 50 | — | 52 | — |
| 141 | M_d(M6) | 50 | — | 53 | — |
| 142 | MHCIsp-M(M2) | 17 | — | 5 | — |
| 143 | MHCIsp-M(M3) | 18 | — | 6 | — |
| 144 | MHCIsp-M(M6) | 20 | — | 7 | — |
| 145 | MHCIsp-M(M2)- | 17 | — | 5 | 26 |
| | MITD | | | | |
| 146 | MHCIsp-M(M3)- | 18 | — | 6 | 25 |
| | MITD | | | | |
| 147 | MHCIsp-M(M6)- | 20 | — | 7 | 24 |
| | MITD | | | | |
| 148 | N(N2) | 50 | — | 9 | — |
| 149 | N(N3) | 50 | — | 10 | — |
| 150 | N(N6) | 50 | — | 11 | — |
| 151 | N_d(N2) | 50 | — | 54 | — |
| 152 | N_d(N3) | 50 | — | 55 | — |
| 153 | N_d(N6) | 50 | — | 56 | — |
| 154 | MHCIsp-N(N2) | 17 | — | 9 | — |
| 155 | MHCIsp-N(N3) | 18 | — | 10 | — |
| 156 | MHCIsp-N(N6) | 19 | — | 11 | — |
| 157 | MHCIsp-N(N2)- | 17 | — | 9 | 27 |
| | MITD | | | | |
| 158 | MHCIsp-N(N3)- | 18 | — | 10 | 28 |
| | MITD | | | | |
| 159 | MHCIsp-N(N6)- | 19 | — | 11 | 29 |
| | MITD | | | | |
| 160 | S(S2) | 50 | — | 13 | — |
| 161 | S(S3) | 50 | — | 14 | — |
| 162 | S(S6) | 50 | — | 15 | — |
| 163 | S_ec(S2) | 50 | — | 57 | — |
| 164 | S_ec(S3) | 50 | — | 58 | — |
| 165 | S_ec(S6) | 50 | — | 59 | — |
| 166 | SII(S2) | 50 | — | 60 | — |
| 167 | SII(S3) | 50 | — | 61 | — |
| 168 | SII(S6) | 50 | — | 62 | — |
| 169 | MHCIsp-S(S2) | 17 | — | 13 | — |
| 170 | MHCIsp-S(S3) | 21 | — | 14 | — |
| 171 | MHCIsp-S(S6) | 22 | — | 15 | — |
| 172 | MHCIsp-S_ec(S2) | 17 | — | 57 | — |
| 173 | MHCIsp-S_ec(S3) | 21 | — | 58 | — |
| 174 | MHCIsp-S_ec(S6) | 22 | — | 59 | — |
| 175 | MHCIsp-SII(S2) | 17 | — | 60 | — |
| 176 | MHCIsp-SII(S3) | 21 | — | 61 | — |
| 177 | MHCIsp-SII(S6) | 22 | — | 62 | — |
| 178 | MHCIsp-S(S2)- | 17 | — | 13 | 30 |
| | MITD | | | | |
| 179 | MHCIsp-S(S3)- | 21 | — | 14 | 31 |
| | MITD | | | | |
| 180 | MHCIsp-S(S6)- | 22 | — | 15 | 32 |
| | MITD | | | | |
| 181 | MHCIsp-S_ec(S2)- | 17 | — | 57 | 30 |
| | MITD | | | | |
| 182 | MHCIsp-S_ec(S3)- | 21 | — | 58 | 31 |
| | MITD | | | | |
| 183 | MHCIsp-S_ec(S6)- | 22 | — | 59 | 32 |
| | MITD | | | | |
| 184 | MHCIsp-SII(S2)- | 17 | — | 60 | 30 |
| | MITD | | | | |
| 185 | MHCIsp-SII(S3)- | 21 | — | 61 | 31 |
| | MITD | | | | |
| 186 | MHCIsp-SII(S6)- | 22 | — | 62 | 32 |
| | MITD | | | | |
| 187 | 7a | 50 | — | 72 | — |
| 188 | 3a | 50 | — | 73 | — |
| 189 | MHCIsp-M(HF1902)- | 17 | — | 63 | 30 |
| | MITD | | | | |
| 190 | N(HF1902) | 50 | — | 64 | — |
| 191 | MHCIsp- | 17 | — | 65 | 30 |
| | SII(HF1902)-MITD | | | | |
| 192 | MHCIsp-M(SH2211)- | 17 | — | 66 | 30 |
| | MITD | | | | |
| 193 | N(SH2211) | 50 | — | 67 | — |
| 194 | MHCIsp-SII(SH2211)- | 17 | — | 68 | 30 |
| | MITD | | | | |
| 195 | MHCIsp-M(2-C11 | 17 | — | 69 | 30 |
| | Re 10276)-MITD | | | | |
| 196 | N(2-C11 Re 10276) | 50 | — | 70 | — |
| 197 | MHCIsp-SII(2-C11 | 17 | — | 71 | 30 |
| | Re 10276)-MITD | | | | |

TABLE 5
Composition of multiple antigen sequences involved in the embodiments of the present disclosure

| | | SEQ ID NO: | | | | | | | | |
|---|---|---|---|---|---|---|---|---|---|---|
| SEQ | | Signal | Tag | | | | Signal | Tag | | |
| ID | | peptide | peptide | Gene | MITD | Linking | peptide | peptide | Gene | MITD |
| NO: | Description | 1 | 1 | 1 | 1 | peptide | 2 | 2 | 2 | 2 |
| 198 | MHCIsp-HA-M- | 20 | 36 | 7 | 24 | 47 | — | 38 | 9 | — |
| | MITD-2A-His-N | | | | | | | | | |
| 199 | MHCIsp-HA-M- | 20 | 36 | 7 | — | 49 | — | — | 9 | 24 |
| | GS-N-MITD | | | | | | | | | |
| 200 | MHCIsp-M- | 20 | — | 7 | 24 | 47 | — | — | 9 | — |
| | MITD-2A-N | | | | | | | | | |
| 201 | MHCIsp-M- | 20 | — | 7 | — | 49 | — | — | 9 | 24 |
| | GS-N-MITD | | | | | | | | | |

Note:
“sp” represents a signal peptide.

It should be noted that, the sequences of the products corresponding to the names in Table 4 and Table 5 are formed by sequentially linking the corresponding sequences in SEQ ID NO:” in a direction of the 5′ end to 3′ end. For example, in Table 5, the sequence of MHCIsp-HA-M-MITD-2A-His-N is formed by linking the signal peptide 1 sequence as set forth in SEQ ID NO: 20, the tag peptide 1 sequence as set forth in SEQ ID NO: 36, the gene 1 sequence as set forth in SEQ ID NO: 7, the MITD 1 sequence as set forth in SEQ ID NO: 24, the linking peptide sequence as set forth in SEQ ID NO: 47, the tag peptide 2 sequence as set forth in SEQ ID NO: 38, and the gene 2 sequence as set forth in SEQ ID NO: 9.

The 3′ end of the signal peptide 1 sequence as set forth in in SEQ ID NO: 20 is linked to the 5′ end of the tag peptide 1 sequence as set forth in SEQ ID NO: 36, the 3′ end of the tag peptide 1 as sequence set forth in SEQ ID NO: 36 is linked to the 5′ end of the gene 1 sequence as set forth in SEQ ID NO: 7, the 3′ end of the gene 1 sequence as set forth in SEQ ID NO: 7 is linked to the 5′ end of the MITD 1 sequence as set forth in SEQ ID NO: 24, the 3′ end of the MITD 1 sequence as set forth in SEQ ID NO: 24 is linked to the 5′ end of the linking peptide sequence as set forth in SEQ ID NO: 47, the 3′ end of the linking peptide sequence as set forth in SEQ ID NO: 47 is linked to the 5′ end of the tag peptide 2 sequence as set forth in SEQ ID NO: 38, and the 3′ end of the tag peptide 2 sequence as set forth in SEQ ID NO: 38 is linked to the 5′ end of the gene 2 sequence as set forth in SEQ ID NO: 9.

The present disclosure is described below with reference to specific examples. It should be noted that these examples are merely illustrative and do not limit the present disclosure in any way. If the specific technologies or conditions are not specified in the examples, they shall be carried out according to the technologies or conditions as described in the literature in the art or according to the product instructions. The reagents and instruments used without indicating the manufacturer are all conventional products that can be purchased commercially.

### Example 1: Selection of Target Sequences

According to this example of the present disclosure, the mRNA sequences were synthesized in vitro and expressed in Crandell Reese feline kidney (CRFK) cells and feline dendritic cells (DCs). Sequences with high expression levels were selected as the final target sequences for vaccine preparation. The specific steps were as follows:
1) Codon optimization was performed on the M, N, and S antigen sequences using a feline universal codon library and a feline mesenteric lymph node codon library, with three optimized sequences generated for each antigen.
2) After sequence synthesis (source: Universal Bio), a vector was constructed. The vector had sequences of the feline-derived 5′ HBB-UTR (SEQ ID NO: 44), 3′ HBA-UTR (SEQ ID NO: 45) and polyA. The target sequence was inserted between the 5′ UTR and 3′ UTR.
3) The plasmid vector was linearized using BsaI enzyme and subjected to phenol-chloroform purification to obtain the linearized template as a template for in vitro transcription of mRNA. T7 enzyme was added to the corresponding reaction system for in vitro transcription at 37° C. Lithium chloride was used to precipitate to obtain purified mRNA.
4) After in vitro transcription into mRNA, the mRNA was transfected into the CRFK cells and the feline dendritic cells (DCs) using a transfection reagent, and sequences with high expression levels in these two feline-derived cell lines were screened.

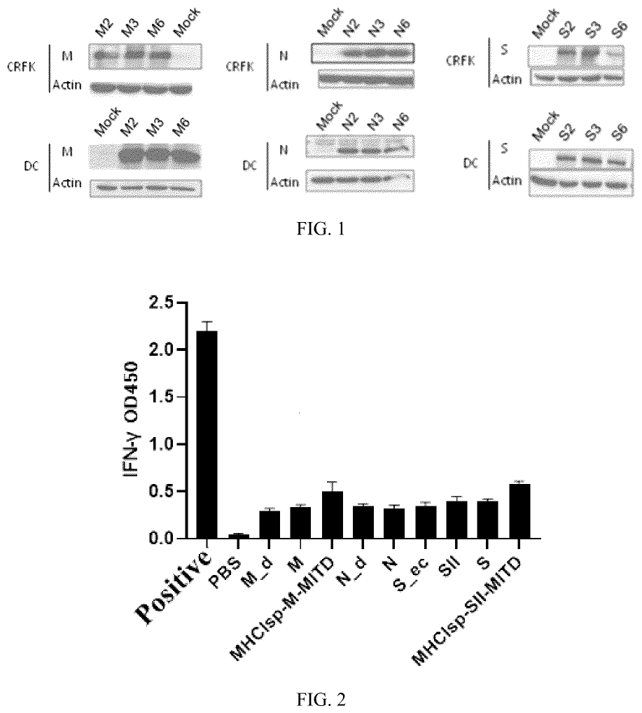

The results, as illustrated in FIG. 1, showed that the target antigen sequences M6 (SEQ ID NO: 7), N2 (SEQ ID NO: 9), and S2 (SEQ ID NO: 13) that can be highly expressed in the two type of cells were successfully screened.

### Example 2: Evaluation of Potency for mRNA Vaccines with Different Antigen Sequence Structures

In this example of the present disclosure, the effectiveness of the vaccine was evaluated by assessing the immune response activated in the subjects following immunization with the mRNA vaccine.

The highly expressed target antigen sequence M (SEQ ID NO: 76) obtained from Example 1 was truncated to obtain the M_d sequence (SEQ ID NO: 79). The MHCIsp and MITD sequences were added before and after M to obtain the MHCIsp-M-MITD sequence (SEQ ID NO: 85). The highly expressed target antigen sequence N (SEQ ID NO: 86) obtained from Example 1 was truncated to obtain the N_d sequence (SEQ ID NO: 89). The highly expressed target antigen sequence S (SEQ ID NO: 98) obtained from Example 1 was truncated to obtain the S_ec sequence (SEQ ID NO: 101) and the SII sequence (SEQ ID NO: 104). The MHCIsp and MITD sequences were added before and after the SII sequence to obtain the MHCIsp-SII-MITD sequence (SEQ ID NO: 122). The sequence composition is shown in Table 4.

After prepared by in vitro transcription, the mRNA was encapsulated with lipid nanoparticles (LNPs) to complete the mRNA vaccine preparation.

The method for in vitro transcription preparation of mRNA is the same as in Example 1.

LNPs encapsulating mRNA were prepared as the mRNA vaccine. The specific steps were as follows:
1) Preparation of lipid solution: The average molecular weight of the liposome system was approximately 620.62. To prepare a 12 mM lipid solution, 42.61 mg of SM-102, 4.52 mg of PEG-DMG, 9.48 mg of DSPC, and 17.86 mg of Chol were weighed, dissolved in 10 mL of anhydrous ethanol, and filter with a 0.22 m filter membrane.
2) The target mRNA was diluted with a citric acid buffer (pH=4). After mixing thoroughly, a rapid nanodrug preparation system (Mingtai) was used to prepare the mRNA vaccine liposome solution with a pre-conditioned flow rate of 1:3 (volume of organic phase X (containing cationic lipid): volume of aqueous phase Y (containing nucleic acid)=1:3). Then, the solution was immediately placed in 30 volumes of PBS, concentrated using a 15 mL ultrafiltration tube with a molecular weight cutoff of 100 kDa, and centrifuged at 3000 rpm for 20 minutes. Finally, the concentrate was subjected to equi-volume dilution with 600 mM sucrose solution (prepared in PBS and filtered through a 0.22 m filter) for storage. The sample was stored at −20° C. for later use.

Subject animals meeting the test criteria were screened through physical examination and laboratory screening tests. The items of physical examination included body temperature and body weight, and the items of screening tests included PCR assay against FIPV, N and S protein-binding antibody assay, and neutralizing antibody assay. The specific experimental steps were as follows:
1) Physical Examination: Seven days prior to immunization, the body temperature and body weight of the kittens were measured every day. The normal body temperature was around 38.5° C., and the body weight of a 1-year-old pet cat was approximately 3 kg.
2) Screening of FIPV-negative cats: PCR assay was used to detect the 7ab gene of FIPV; and the ELISA assay was used to detect binding antibodies in cat serum using the N and S proteins as antigens, and pseudovirus neutralization assay was used to detect neutralization antibodies against FIPV.

The screened subject animals were immunized with the target mRNA encapsulated by LNP. The immunization schedules were as follows:
primary immunization on Day 0 (D0), secondary immunization on Day 14 (D14), and tertiary immunization on Day 28 (D28). PBMCs were extracted on D14 after the tertiary booster immunization.

The PBMCs of the subjects were extracted using the Feline Peripheral Blood Mononuclear Cell Separation Solution Kit (Solarbio) and then stimulated with a synthetic polypeptide library of the corresponding target antigen. The supernatant was collected and then the feline IFN-γ secretion level was detected using the Feline IFN-γ ELISA Detection Kit (MABTECH).

After immunization with the target mRNA encapsulated by LNP in each group, the PBMCs of the subjects were extracted for detection. The results, as illustrated in FIG. 2, showed that PBMCs in all groups exhibited activation of immune response. Compared with the experimental group immunized with M, the experimental group immunized with M_d showed a similar level of IFN-γ and a similar activated immune response, with M being slightly superior to M_d. Compared with the experimental group immunized with N, the experimental group immunized with N_d had a similar level of IFN-γ and a similar activated immune response, with N being slightly superior to N_d. Compared with the experimental group immunized with S, the experimental groups immunized with S_ec and SII each showed a similar level of IFN-γ and a similar activated immune response, with SII being slightly superior to S and S_ec. Compared with the experimental groups immunized with M and SII, the experimental groups immunized with MHCIsp-M-MITD and MHCIsp-SII-MITD each showed an increased level of IFN-γ and a stronger activated immune response.

The above results showed that mRNA vaccines expressing the target antigens M, N, and S can effectively activate the immune response of subjects against FIPV. Among mRNA vaccines with different antigen structures, those expressing the target antigens MHCIsp-M-MITD and MHCIsp-SII-MITD with added MHCIsp and MITD can activate a stronger immune response compared with the groups without the addition of MHCIsp and MITD.

### Example 3: Evaluation of Potency for mRNA Vaccines with Different Antigen Combinations

In this example, the effectiveness of the mRNA vaccine was evaluated by assessing the immune response activated in subjects after immunization with the vaccine containing different antigen combinations.

The mRNAs expressing MHCIsp-M-MITD (SEQ ID NO: 85), N (SEQ ID NO: 86), and MHCIsp-SII-MITD (SEQ ID NO: 122) were prepared, respectively. Then, according to examples of the present disclosure, the mRNAs of all experimental groups were mixed and encapsulated by LNPs. The sequence composition is shown in Table 4.

The method for preparing the mRNA vaccine, the method for screening and immunizing the subjects, the method for extracting PBMCs from the subjects, and the method for detecting IFN-γ were the same as those in Example 2.

After immunization with the target mRNA encapsulated by LNP in each group, the PBMCs of the subjects were extracted for detection. The results, as illustrated in FIG. 3, showed that PBMCs in all groups exhibited activation of immune response. When evaluated by IFN-7, the (M+N+SII) group showed an immune effect that exceeded that of any single-antigen group or dual-antigen group when acting alone. That is, the activation level of IFN-γ followed the order: (M+N+SII) group>M group+N group+SII group, (M+N+SII) group>M group+(N+SII) group, (M+N+SII) group>N group+(M+SII) group, and (M+N+SII) group>SII group+(M+N) group.

The above results showed that the mRNA vaccines expressing different combinations of the target antigens M, N, and SII can effectively activate the immune response against FIPV in the subjects; and the (M+N+SII) combination exhibited an immune effect that exceeds that of any single-antigen group or dual-antigen group when acting alone.

### Example 4: Evaluation of Viral Challenge Protection Efficacy of mRNA Vaccines with Different Antigen Combinations

In this example, the effectiveness of the mRNA vaccine was evaluated by assessing changes in physiological indicators such as body temperature and body weight, as well as survival rates of subjects after viral challenge following immunization with the vaccine.

The mRNAs expressing MHCIsp-M-MITD (SEQ ID NO: 85), N (SEQ ID NO: 86), MHCIsp-S_ec-MITD (SEQ ID NO: 119), MHCIsp-SII-MITD (SEQ ID NO: 122), His-7a (SEQ ID NO: 125), and Flag-3a (SEQ ID NO: 126) were prepared, respectively. Then, according to examples of the present disclosure, the mRNAs of all experimental groups were mixed and encapsulated with LNPs. The sequence composition is shown in Table 4.

The method for preparing the mRNA vaccine and the method for screening the subjects were the same as those in Example 2.

The screened subjects were immunized with the target mRNA encapsulated by LNPs. The immunization schedule was as follows:
primary immunization on Day 0 (D0), booster immunization on Day 21 (D21), and viral challenge on Day 28 (D28) after the booster immunization; the challenge strain was QS-1146; and 5 kittens per group.

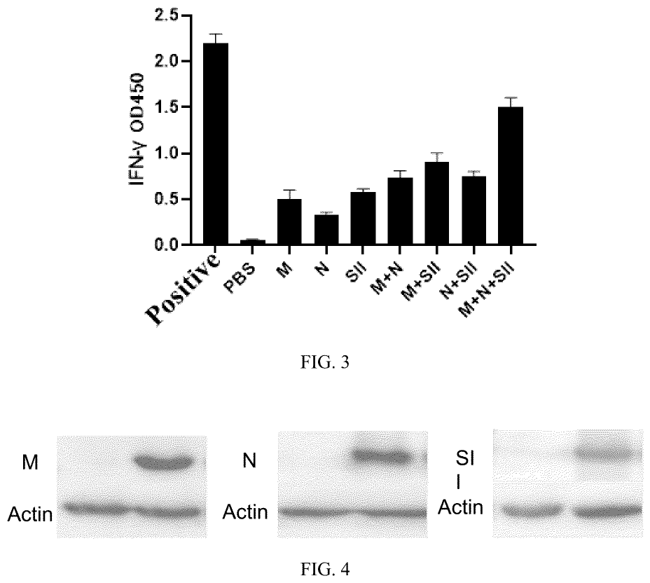

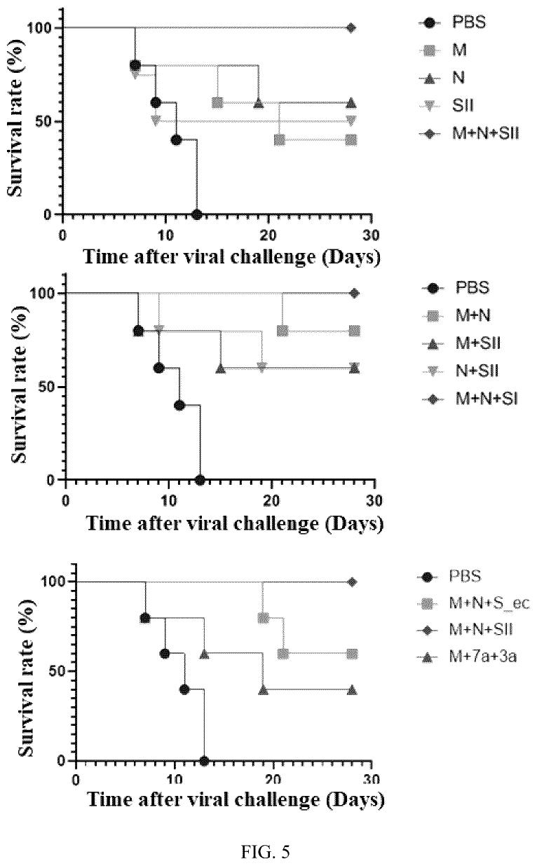

The target mRNA encapsulated by LNP was transfected into the CRFK cells. The transfected CRFK cells were collected after 24 hours, and subjected to a Western blotting assay to detect protein expression. The results, as illustrated in FIG. 4, showed that all mRNAs were normally expressed. After immunization and viral challenge with the target mRNA encapsulated by LNP in each group, all five kittens in the PBS group successively developed symptoms of fever and weight loss. Post-mortem dissection revealed that these kittens had typical pathological lesions of feline infectious peritonitis. In contrast, the kittens in the immunized groups (immunized with mRNA expressing M, N, or SII antigens, or a combination thereof) showed significantly improved physiological indicators, and all kittens in the M+N+SII group remained normal. The survival rate, as illustrated in FIG. 5, showed that immunization with mRNA expressing the individual M, N, and SII antigens improved the survival rate following viral challenge. In addition, the combination of multiple antigens can further improve the survival rate after viral challenge. The survival rate of the (M+7a+3a) group after viral challenge was similar to that of the M group, while the survival rate of the M+N+SII group reached 100% after viral challenge. When evaluated by the protection rate of viral challenge, the (M+N+SII) group exhibited an immune effect that exceeded that of any single-antigen group and any dual-antigen group when acting alone. That is, the maximum protection rate of each single-antigen group and each dual-antigen group (when acting alone) was only 80%, but the (M+N+SII) group could reach a 100% protection rate. The survival rate of the (M+N+S_ec) group after viral challenge decreased significantly compared with that of the (M+N) group and the (M+N+SII) group, indicating that SII can improve the protective effect of the multi-antigen combination vaccine compared with S_ec.

The above results showed that mRNAs expressing the individual M, N, and SII antigens or a combination thereof, when used as the vaccine, exhibited a good immune effect against FIPV, significantly improving various physiological indicators and the survival rate, with the combination of M+N+SII yielding the best effect.

### Example 5: Evaluation of Viral Challenge Protection Efficacy of Multi-Antigen mRNA Vaccines Derived from Different Virus Strains

In this example, the effectiveness of multi-antigen mRNA vaccines derived from different strains was evaluated.

The mRNAs expressing MHCIsp-M(HF1902)-MITD (SEQ ID NO: 127), N(HF1902) (SEQ ID NO: 128), MHCIsp-SII(HF1902)-MITD (SEQ ID NO: 129), MHCIsp-M(SH2211)-MITD (SEQ ID NO: 130), N(SH2211) (SEQ ID NO: 131), and MHCIsp-SII(SH2211)-MITD (SEQ ID NO: 132) were prepared, respectively. Then, according to examples of the present disclosure, the mRNAs of all experimental groups were mixed and encapsulated by LNPs. The sequence composition is shown in Table 4.

The method for preparing the mRNA vaccine, the method for screening the subjects, the immunization schedule, and the evaluation method were the same as those in Example 4.

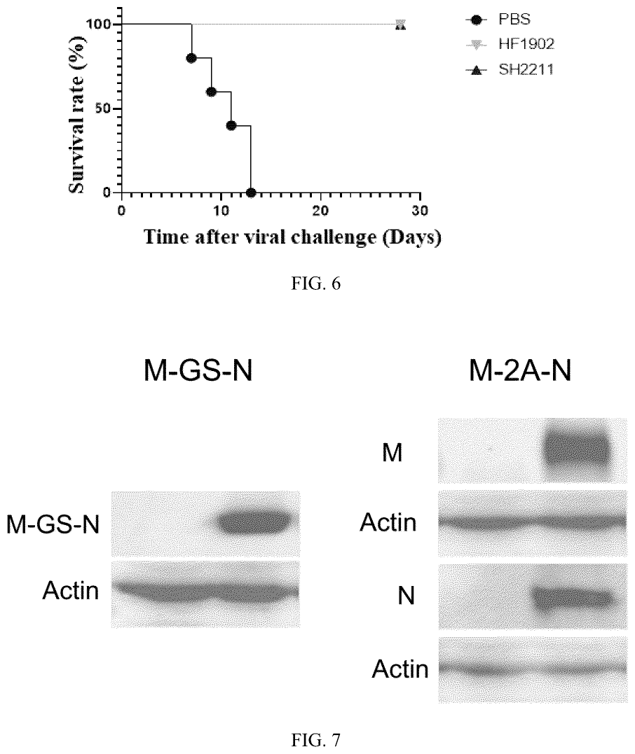

After immunization and viral challenge with the target mRNA encapsulated by LNP in each group, all five kittens in the PBS group successively developed symptoms of fever and weight loss. Post-mortem dissection revealed that these kittens had typical pathological lesions of feline infectious peritonitis. In contrast, the kittens in the immunized groups (with antigens derived from the HF1902 and SH2211 strains) showed significantly improved physiological indicators, and their survival rates are shown in FIG. 6.

The above results showed that multi-antigen mRNA vaccines derived from different strains all exhibited good immune efficacy against FIPV

### Example 6: Evaluation of Viral Challenge Protection Efficacy of Multi-Antigen mRNA Vaccines with Different Linkage Modes

In this example of the present disclosure, the effectiveness of multi-antigen mRNA vaccines with different linkage modes was evaluated.

The mRNAs expressing MHCIsp-M-MITD (SEQ ID NO: 85), N (SEQ ID NO: 86), MHCIsp-M-MITD-2A-N(SEQ ID NO: 198), and MHCIsp-M-GS-N-MITD (SEQ ID NO: 199) were prepared, respectively. Then, according to examples of the present disclosure, the mRNAs of all experimental groups were mixed and encapsulated by LNPs. The sequence composition is shown in Table 4 and Table 5.

The methods for preparing the mRNA vaccine, the method for screening the subjects, the immunization procedure, and the evaluation method were the same as those in Example 4.

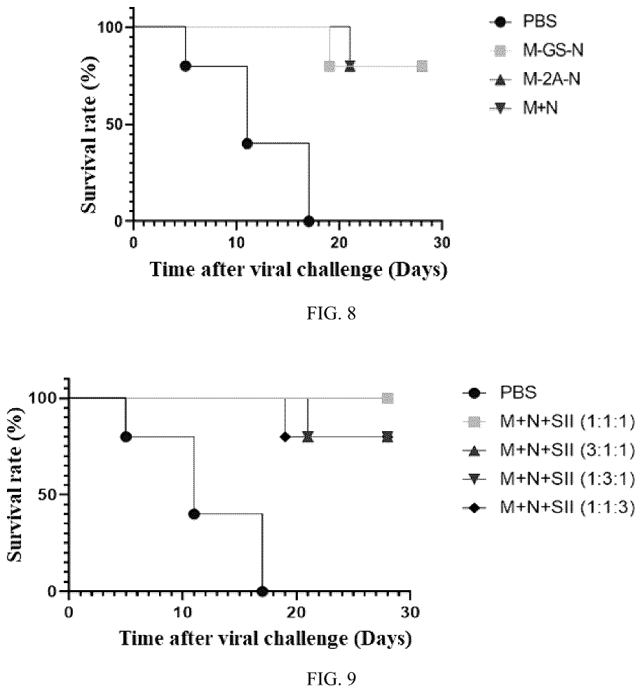

The expression detection results of the target mRNA after encapsulated by LNP were shown in FIG. 7, and all mRNAs were normally expressed. After immunization and viral challenge, the multi-antigen mRNA vaccines with different linkage modes all effectively improved the physiological indicators and survival rate of the subject animals after the viral challenge. The survival rate was shown in FIG. 8, and there were no significant differences between the different linkage modes.

The above results showed that the isolated single-antigen mRNA vaccines (serving as a pharmaceutical composition) and the multi-antigen mRNA vaccines linked by different linking peptides both exhibited good immune effects against FIPV

### Example 4: Evaluation of Viral Challenge Protection Efficacy of Multi-Antigen mRNA Vaccines with Different Ratios

In this example, the effectiveness of multi-antigen mRNA vaccines at different ratios was evaluated.

The mRNAs expressing MHCIsp-M-MITD (SEQ ID NO: 85), N (SEQ ID NO: 86), and MHCIsp-SII-MITD (SEQ ID NO: 122) were prepared, respectively. Then, according to examples of the present disclosure, the mRNAs of all experimental groups were mixed and encapsulated by LNPs. The sequence composition is shown in Table 4.

The methods for preparing the mRNA vaccine, the method for screening the subjects, the immunization procedure, and the evaluation method were the same as those in Example 4.After immunization and viral challenge, the multi-antigen mRNA vaccines with different ratios all effectively improved the physiological indicators and survival rate of the subject animals. The survival rate was shown in FIG. 9.

The above results showed that multi-antigen mRNA vaccines with different ratios can all exhibit good immune effects against FIPV.

### Example 8: Evaluation of Viral Challenge Protection Efficacy of Multi-Antigen mRNA Vaccines Against Different Prevalent Strains

In this example, the effectiveness of the multi-antigen mRNA vaccine against different prevalent strains of FIPV was evaluated.

The mRNAs expressing MHCIsp-M-MITD (SEQ ID NO: 85), N (SEQ ID NO: 86), and MHCIsp-SII-MITD (SEQ ID NO: 122) were prepared, respectively. Then, according to examples of the present disclosure, the mRNAs of all experimental groups were mixed and encapsulated by LNPs. The sequence composition is shown in Table 4.

The method for preparing the mRNA vaccine, the method for screening the subjects, the immunization procedure, and the evaluation method were the same as those in Example 4. The viral challenge strains were QS, 79-1146, 1HF1902, and SH2211.

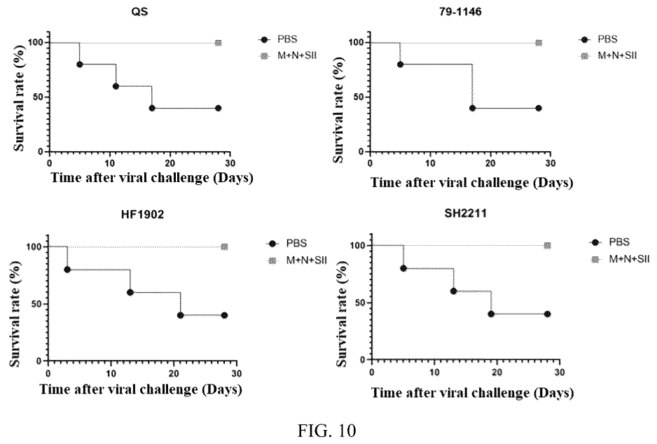

After immunization and viral challenge, the multi-antigen mRNA vaccine can effectively improve the physiological indicators and survival rate of the subject animals against different prevalent strains of FIPV. The survival rate was shown in FIG. 10.

The above results showed that the multi-antigen mRNA vaccine exhibited good immune effects against different prevalent strains of FIPV

In the description of the present specification, reference to the terms such as “an embodiment,” “some embodiments,” “an example,” “a specific example,” or “some examples” mean that a particular feature, structure, material, or characteristic described in connection with the embodiment or example is included in at least one embodiment or example of the present disclosure. In the present specification, the illustrative expressions of the above terms do not necessarily refer to the same embodiment or example. Furthermore, the particular features, structures, materials, or characteristics described may be combined in any suitable manner in one or more embodiments or examples. In addition, without mutual contradiction, those skilled in the art can combine and integrate the different embodiments or examples described in this specification, as well as the features of different embodiments or examples.

Although the embodiments of the present disclosure have been shown and described above, it should be understood that the above-described embodiments are illustrative and should not be construed as limiting the present disclosure. Those skilled in the art can make changes, modifications, substitutions, and variations to the above-described embodiments within the scope of the present disclosure.

What is claimed is:

1. A pharmaceutical formulation, comprising a nucleic acid fragment, the nucleic acid fragment being mRNA and being a linear molecule and comprising at least one of a first nucleic acid fragment, a second nucleic acid fragment, or a third nucleic acid fragment, wherein:
the first nucleic acid fragment encodes an M protein of feline infectious peritonitis virus;
the second nucleic acid fragment encodes an N protein of feline infectious peritonitis virus;
the third nucleic acid fragment encodes an S protein, S_ec protein, or SII protein of feline infectious peritonitis virus; and
the first nucleic acid fragment, the second nucleic acid fragment, and the third nucleic acid fragment are linked or not linked.

2. The pharmaceutical formulation according to claim 1, wherein the first nucleic acid fragment, the second nucleic acid fragment, and the third nucleic acid fragment are not linked, optionally, a mass ratio of the first nucleic acid fragment to the second nucleic acid fragment is 10:1 to 1:10;
optionally, a mass ratio of the second nucleic acid fragment to the third nucleic acid fragment is 10:1 to 1:10;
optionally, a mass ratio of the first nucleic acid fragment, the second nucleic acid fragment, and the third nucleic acid fragment is 1:1:1, or
wherein the first nucleic acid fragment, the second nucleic acid fragment, and the third nucleic acid fragment are linked,
optionally, a mass ratio of the first nucleic acid fragment to the second nucleic acid fragment is 10:1 to 1:10;
optionally, a mass ratio of the second nucleic acid fragment to the third nucleic acid fragment is 10:1 to 1:10;
optionally, a mass ratio of the first nucleic acid fragment, the second nucleic acid fragment, and the third nucleic acid fragment is 1:1:1, or
wherein optionally, the third nucleic acid fragment is selected from the SII protein.

3. A method for preparing the pharmaceutical formulation according to claim 1, the method comprising:
mixing a first nucleic acid fragment, a second nucleic acid fragment, and a third nucleic acid fragment in a predetermined ratio to obtain the pharmaceutical formulation, wherein:
the first nucleic acid fragment encodes an M protein of feline infectious peritonitis virus;
the second nucleic acid fragment encodes an N protein of feline infectious peritonitis virus;
the third nucleic acid fragment encodes an S protein, S_ec protein, or SII protein of feline infectious peritonitis virus; and
the first nucleic acid fragment, the second nucleic acid fragment, and the third nucleic acid fragment are each mRNA,
optionally, a mass ratio of the first nucleic acid fragment to the second nucleic acid fragment is 10:1 to 1:10,
optionally, a mass ratio of the second nucleic acid fragment to the third nucleic acid fragment is 10:1 to 1:10,
optionally, a mass ratio of the first nucleic acid fragment, the second nucleic acid fragment, and the third nucleic acid fragment is 1:1:1.

4. An isolated nucleic acid molecule, comprising:
a first nucleic acid fragment encoding an M protein of feline infectious peritonitis virus;
a second nucleic acid fragment encoding an N protein of feline infectious peritonitis virus; and
a third nucleic acid fragment encoding an S protein, Sec protein, or SII protein of feline infectious peritonitis virus,
wherein the first nucleic acid fragment, the second nucleic acid fragment, and the third nucleic acid fragment are linked or not linked; and
wherein the nucleic acid molecule is mRNA and is a linear molecule.

5. The pharmaceutical formulation according to claim 2, wherein:
the first nucleic acid fragment is linked to the second nucleic acid fragment, and the third nucleic acid fragment is not linked to each of the first nucleic acid fragment and the second nucleic acid fragment; or
the first nucleic acid fragment is linked to the third nucleic acid fragment, and the second nucleic acid fragment is not linked to each of the first nucleic acid fragment and the third nucleic acid fragment;
or
the second nucleic acid fragment is linked to the third nucleic acid fragment, and the first nucleic acid fragment is not linked to each of the second nucleic acid fragment and the third nucleic acid fragment;
optionally, the 3′ end of the first nucleic acid fragment is linked to the 5′ end of the second nucleic acid fragment, and the third nucleic acid fragment is not linked to each of the first nucleic acid fragment and the second nucleic acid fragment; or
the 3′ end of the second nucleic acid fragment is linked to the 5′ end of the first nucleic acid fragment, and the third nucleic acid fragment is not linked to each of the first nucleic acid fragment and the second nucleic acid fragment; or
the 3′ end of the first nucleic acid fragment is linked to the 5′ end of the third nucleic acid fragment, and the second nucleic acid fragment is not linked to each of the first nucleic acid fragment and the third nucleic acid fragment; or
the 3′ end of the third nucleic acid fragment is linked to the 5′ end of the first nucleic acid fragment, and the second nucleic acid fragment is not linked to each of the first nucleic acid fragment and the third nucleic acid fragment; or
the 3′ end of the second nucleic acid fragment is linked to the 5′ end of the third nucleic acid fragment, and the first nucleic acid fragment is not linked to each of the second nucleic acid fragment and the third nucleic acid fragment; or
the 3′ end of the third nucleic acid fragment is linked to the 5′ end of the second nucleic acid fragment, and the first nucleic acid fragment is not linked to each of the second nucleic acid fragment and the third nucleic acid fragment; or
the 3′ end of the first nucleic acid fragment is linked to the 5′ end of the second nucleic acid fragment, and the 3′ end of the second nucleic acid fragment is linked to the 5′ end of the third nucleic acid fragment; or
the 3′ end of the first nucleic acid fragment is linked to the 5′ end of the third nucleic acid fragment, and the 3′ end of the third nucleic acid fragment is linked to the 5′ end of the second nucleic acid fragment; or
the 3′ end of the second nucleic acid fragment is linked to the 5′ end of the first nucleic acid fragment, and the 3′ end of the first nucleic acid fragment is linked to the 5′ end of the third nucleic acid fragment; or
the 3′ end of the second nucleic acid fragment is linked to the 5′ end of the third nucleic acid fragment, and the 3′ end of the third nucleic acid fragment is linked to the 5′ end of the first nucleic acid fragment; or
the 3′ end of the third nucleic acid fragment is linked to the 5′ end of the first nucleic acid fragment, and the 3′ end of the first nucleic acid fragment is linked to the 5′ end of the second nucleic acid fragment; or
the 3′ end of the third nucleic acid fragment is linked to the 5′ end of the second nucleic acid fragment, and the 3′ end of the second nucleic acid fragment is linked to the 5′ end of the first nucleic acid fragment.

6. The pharmaceutical formulation according to claim 1, wherein the M protein has an amino acid sequence with at least 89% homology to the amino acid sequence as set forth in SEQ ID NO: 1,
optionally, the M protein has the amino acid sequence with at least 89% homology to the amino acid sequence as set forth in SEQ ID NO: 1, wherein:
an amino acid at site 90 is Y;
an amino acid at site 102 is V;
an amino acid at site 120 is I;
an amino acid at site 144 is A; and
an amino acid at site 180 is L,
optionally, the M protein has the amino acid sequence as set forth in SEQ ID NO: 1.
optionally, the N protein has an amino acid sequence with at least 91% homology to the amino acid sequence as set forth in SEQ ID NO: 2,
optionally, the N protein has the amino acid sequence as set forth in SEQ ID NO: 2,
optionally, the S protein has an amino acid sequence with at least 45% homology to the amino acid sequence as set forth in SEQ ID NO: 3,
optionally, the S protein has the amino acid sequence with at least 45% homology to the amino acid sequence as set forth in SEQ ID NO: 3, wherein:
an amino acid at site 515 is V;
an amino acid at site 577 is Q;
an amino acid at site 1385 is V;
an amino acid at site 1386 is V;
an amino acid at site 1397 is F; and
an amino acid at site 1415 is I,
optionally, the S protein has the amino acid sequence as set forth in SEQ ID NO: 3,
optionally, the Sec protein has an amino acid sequence with at least 43% homology to amino acids at sites 1 to 1374 of the amino acid sequence as set forth in SEQ ID NO: 3,
optionally, the Sec protein has the amino acid sequence with at least 43% homology to the amino acids at sites 1 to 1374 of the amino acid sequence as set forth in SEQ ID NO: 3, wherein an amino acid at site 515 is V, and wherein an amino acid at site 577 is Q,
optionally, the Sec protein has an amino acid sequence of sites 1 to 1374 of SEQ ID NO: 3,
optionally, the SII protein has an amino acid sequence with at least 62% homology to amino acids at sites 782 to 1433 of the amino acid sequence as set forth in SEQ ID NO: 3,
optionally, the SII protein has the amino acid sequence with at least 62% homology to amino acids at sites 782 to 1433 of the amino acid sequence as set forth in SEQ ID NO: 3, wherein:
an amino acid at site 1385 is V;
an amino acid at site 1386 is V;
an amino acid at site 1397 is F; and
an amino acid at site 1415 is I,
optionally, the SII protein has an amino acid sequence of sites 782 to 1433 of SEQ ID NO: 3.
optionally, the first nucleic acid fragment has a nucleotide sequence with at least 67% homology to the sequence as set forth in any one of SEQ ID NO: 4 to SEQ ID NO: 7,
optionally, the first nucleic acid fragment has the nucleotide sequence as set forth in any one of SEQ ID NO: 4 to SEQ ID NO: 7, SEQ ID NO: 51 to SEQ ID NO: 53, SEQ ID NO: 63, SEQ ID NO:
66, and SEQ ID NO: 69,
optionally, the second nucleic acid fragment has a nucleotide sequence with at least 70% homology to the sequence as set forth in any one of SEQ ID NO: 8 to SEQ ID NO: 11,
optionally, the second nucleic acid fragment has a nucleotide sequence as set forth in any one of SEQ ID NO: 8 to SEQ ID NO: 11, SEQ ID NO: 54 to SEQ ID NO: 56, SEQ ID NO: 64, SEQ ID NO: 67, and SEQ ID NO: 70,
optionally, the third nucleic acid fragment has a nucleotide sequence with at least 51% homology to nucleotides at sites 1 to 4122 of the sequence as set forth in any one of SEQ ID NO: 12 to SEQ ID NO: 15,
optionally, the third nucleic acid fragment has a nucleotide sequence of sites 1 to 4122 of the sequence as set forth in any one of SEQ ID NO: 12 to SEQ ID NO: 15,
optionally, the third nucleic acid fragment has a nucleotide sequence with at least 57% homology to nucleotides of sites 2344 to 4299 of any one of SEQ ID NO: 12 to SEQ ID NO: 15,
optionally, the third nucleic acid fragment has a nucleotide sequence of sites 2344 to 4299 of any one of SEQ ID NO: 12 to SEQ ID NO: 15,
optionally, the third nucleic acid fragment has a nucleotide sequence with at least 51% homology to the sequence as set forth in any one of SEQ ID NO: 12 to SEQ ID NO: 15,
optionally, the third nucleic acid fragment has a nucleotide sequence as set forth in any one of SEQ ID NO: 12 to SEQ ID NO: 15, SEQ ID NO: 57 to SEQ ID NO: 62, SEQ ID NO: 65, SEQ ID NO: 68, and SEQ ID NO: 71,
optionally, the nucleic acid fragment has a nucleotide sequence shown in Table 4 or Table 5.

7. The pharmaceutical formulation according to claim 1, further comprising a fourth nucleic acid fragment, wherein the fourth nucleic acid fragment encodes a signal peptide sequence of MHC I or a sequence having a similar function to a signal peptide of MHC I,
optionally, the signal peptide sequence of MHC I comprises no transmembrane region,
optionally, the signal peptide sequence of MHC I has the amino acid sequence as set forth in SEQ ID NO: 16,
optionally, the fourth nucleic acid fragment has the nucleotide sequence as set forth in any one of SEQ ID NO: 17 to SEQ ID NO: 22,
optionally, the fourth nucleic acid fragment is located at the 5′ end of the nucleic acid fragment or the 5′ end of the nucleic acid molecule.

8. The pharmaceutical formulation according to claim 1, further comprising a fifth nucleic acid fragment, wherein the fifth nucleic acid fragment encodes an MITD sequence or a sequence having a similar function to MITD,
optionally, the MITD sequence has the amino acid sequence as set forth in SEQ ID NO: 23,
optionally, the fifth nucleic acid fragment has the nucleotide sequence as set forth in any one of SEQ ID NO: 24 to SEQ ID NO: 32.
optionally, the fifth nucleic acid fragment is located at the 3′ end of the nucleic acid fragment or the 3′ end of the nucleic acid molecule.

9. The nucleic acid molecule according to claim 4, wherein:
the first nucleic acid fragment is linked to the second nucleic acid fragment, and the third nucleic acid fragment is not linked to each of the first nucleic acid fragment and the second nucleic acid fragment; or
the first nucleic acid fragment is linked to the third nucleic acid fragment, and the second nucleic acid fragment is not linked to each of the first nucleic acid fragment and the third nucleic acid fragment; or
the second nucleic acid fragment is linked to the third nucleic acid fragment, and the first nucleic acid fragment is not linked to each of the second nucleic acid fragment and the third nucleic acid fragment;
optionally, the 3′ end of the first nucleic acid fragment is linked to the 5′ end of the second nucleic acid fragment, and the third nucleic acid fragment is not linked to each of the first nucleic acid fragment and the second nucleic acid fragment; or
the 3′ end of the second nucleic acid fragment is linked to the 5′ end of the first nucleic acid fragment, and the third nucleic acid fragment is not linked to each of the first nucleic acid fragment and the second nucleic acid fragment; or
the 3′ end of the first nucleic acid fragment is linked to the 5′ end of the third nucleic acid fragment, and the second nucleic acid fragment is not linked to each of the first nucleic acid fragment and the third nucleic acid fragment; or
the 3′ end of the third nucleic acid fragment is linked to the 5′ end of the first nucleic acid fragment, and the second nucleic acid fragment is not linked to each of the first nucleic acid fragment and the third nucleic acid fragment; or
the 3′ end of the second nucleic acid fragment is linked to the 5′ end of the third nucleic acid fragment, and the first nucleic acid fragment is not linked to each of the second nucleic acid fragment and the third nucleic acid fragment; or
the 3′ end of the third nucleic acid fragment is linked to the 5′ end of the second nucleic acid fragment, and the first nucleic acid fragment is not linked to each of the second nucleic acid fragment and the third nucleic acid fragment; or
the 3′ end of the first nucleic acid fragment is linked to the 5′ end of the second nucleic acid fragment, and the 3′ end of the second nucleic acid fragment is linked to the 5′ end of the third nucleic acid fragment; or
the 3′ end of the first nucleic acid fragment is linked to the 5′ end of the third nucleic acid fragment, and the 3′ end of the third nucleic acid fragment is linked to the 5′ end of the second nucleic acid fragment; or
the 3′ end of the second nucleic acid fragment is linked to the 5′ end of the first nucleic acid fragment, and the 3′ end of the first nucleic acid fragment is linked to the 5′ end of the third nucleic acid fragment; or
the 3′ end of the second nucleic acid fragment is linked to the 5′ end of the third nucleic acid fragment, and the 3′ end of the third nucleic acid fragment is linked to the 5′ end of the first nucleic acid fragment; or
the 3′ end of the third nucleic acid fragment is linked to the 5′ end of the first nucleic acid fragment, and the 3′ end of the first nucleic acid fragment is linked to the 5′ end of the second nucleic acid fragment; or
the 3′ end of the third nucleic acid fragment is linked to the 5′ end of the second nucleic acid fragment, and the 3′ end of the second nucleic acid fragment is linked to the 5′ end of the first nucleic acid fragment.

10. The nucleic acid molecule according to claim 4, wherein the M protein has an amino acid sequence with at least 89% homology to the amino acid sequence as set forth in SEQ ID NO: 1, optionally, the M protein has the amino acid sequence with at least 89% homology to the amino acid sequence as set forth in SEQ ID NO: 1, wherein:
an amino acid at site 90 is Y;
an amino acid at site 102 is V;
an amino acid at site 120 is I;
an amino acid at site 144 is A; and
an amino acid at site 180 is L,
optionally, the M protein has the amino acid sequence as set forth in SEQ ID NO: 1.
optionally, the N protein has an amino acid sequence with at least 91% homology to the amino acid sequence as set forth in SEQ ID NO: 2,
optionally, the N protein has the amino acid sequence as set forth in SEQ ID NO: 2,
optionally, the S protein has an amino acid sequence with at least 45% homology to the amino acid sequence as set forth in SEQ ID NO: 3,
optionally, the S protein has the amino acid sequence with at least 45% homology to the amino acid sequence as set forth in SEQ ID NO: 3, wherein:
an amino acid at site 515 is V;
an amino acid at site 577 is Q;
an amino acid at site 1385 is V;
an amino acid at site 1386 is V;
an amino acid at site 1397 is F; and
an amino acid at site 1415 is I,
optionally, the S protein has the amino acid sequence as set forth in SEQ ID NO: 3,
optionally, the Sec protein has an amino acid sequence with at least 43% homology to amino acids at sites 1 to 1374 of the amino acid sequence as set forth in SEQ ID NO: 3,
optionally, the Sec protein has the amino acid sequence with at least 43% homology to the amino acids at sites 1 to 1374 of the amino acid sequence as set forth in SEQ ID NO: 3, wherein an amino acid at site 515 is V, and wherein an amino acid at site 577 is Q,
optionally, the Sec protein has an amino acid sequence of sites 1 to 1374 of SEQ ID NO: 3,
optionally, the SII protein has an amino acid sequence with at least 62% homology to amino acids at sites 782 to 1433 of the amino acid sequence as set forth in SEQ ID NO: 3,
optionally, the SII protein has the amino acid sequence with at least 62% homology to amino acids at sites 782 to 1433 of the amino acid sequence as set forth in SEQ ID NO: 3, wherein:
an amino acid at site 1385 is V;
an amino acid at site 1386 is V;
an amino acid at site 1397 is F; and
an amino acid at site 1415 is I,
optionally, the SII protein has an amino acid sequence of sites 782 to 1433 of SEQ ID NO: 3.
optionally, the first nucleic acid fragment has a nucleotide sequence with at least 67% homology to the sequence as set forth in any one of SEQ ID NO: 4 to SEQ ID NO: 7,
optionally, the first nucleic acid fragment has the nucleotide sequence as set forth in any one of SEQ ID NO: 4 to SEQ ID NO: 7, SEQ ID NO: 51 to SEQ ID NO: 53, SEQ ID NO: 63, SEQ ID NO: 66, and SEQ ID NO: 69,
optionally, the second nucleic acid fragment has a nucleotide sequence with at least 70% homology to the sequence as set forth in any one of SEQ ID NO: 8 to SEQ ID NO: 11,
optionally, the second nucleic acid fragment has a nucleotide sequence as set forth in any one of SEQ ID NO: 8 to SEQ ID NO: 11, SEQ ID NO: 54 to SEQ ID NO: 56, SEQ ID NO: 64, SEQ ID NO: 67, and SEQ ID NO: 70,
optionally, the third nucleic acid fragment has a nucleotide sequence with at least 51% homology to nucleotides at sites 1 to 4122 of the sequence as set forth in any one of SEQ ID NO: 12 to SEQ ID NO: 15,
optionally, the third nucleic acid fragment has a nucleotide sequence of sites 1 to 4122 of the sequence as set forth in any one of SEQ ID NO: 12 to SEQ ID NO: 15,
optionally, the third nucleic acid fragment has a nucleotide sequence with at least 57% homology to nucleotides of sites 2344 to 4299 of any one of SEQ ID NO: 12 to SEQ ID NO: 15,
optionally, the third nucleic acid fragment has a nucleotide sequence of sites 2344 to 4299 of any one of SEQ ID NO: 12 to SEQ ID NO: 15,
optionally, the third nucleic acid fragment has a nucleotide sequence with at least 51% homology to the sequence as set forth in any one of SEQ ID NO: 12 to SEQ ID NO: 15,
optionally, the third nucleic acid fragment has a nucleotide sequence as set forth in any one of SEQ ID NO: 12 to SEQ ID NO: 15, SEQ ID NO: 57 to SEQ ID NO: 62, SEQ ID NO: 65, SEQ ID NO: 68, and SEQ ID NO: 71,
optionally, the nucleic acid fragment has a nucleotide sequence shown in Table 4 or Table 5.

11. The nucleic acid molecule according to claim 4, further comprising a fourth nucleic acid fragment, wherein the fourth nucleic acid fragment encodes a signal peptide sequence of MHC I or a sequence having a similar function to a signal peptide of MHC I,
optionally, the signal peptide sequence of MHC I comprises no transmembrane region,
optionally, the signal peptide sequence of MHC I has the amino acid sequence as set forth in SEQ ID NO: 16,
optionally, the fourth nucleic acid fragment has the nucleotide sequence as set forth in any one of SEQ ID NO: 17 to SEQ ID NO: 22,
optionally, the fourth nucleic acid fragment is located at the 5′ end of the nucleic acid fragment or the 5′ end of the nucleic acid molecule.

12. The nucleic acid molecule according to claim 4, further comprising a fifth nucleic acid fragment, wherein the fifth nucleic acid fragment encodes an MITD sequence or a sequence having a similar function to MITD,
optionally, the MITD sequence has the amino acid sequence as set forth in SEQ ID NO: 23,
optionally, the fifth nucleic acid fragment has the nucleotide sequence as set forth in any one of SEQ ID NO: 24 to SEQ ID NO: 32.
optionally, the fifth nucleic acid fragment is located at the 3′ end of the nucleic acid fragment or the 3′ end of the nucleic acid molecule.

13. The pharmaceutical formulation according to claim 1, further comprising a pharmaceutical carrier, wherein the pharmaceutical carrier comprises at least one of a liposome, an exosome, a polymer carrier, a viral vector, or a nanoparticle.

14. An expression vector, carrying the nucleic acid molecule according to claim 4, preferably the expression vector is a non-viral vector.

15. A recombinant virus, carrying the nucleic acid molecule according to claim 4.

16. A liposome, comprising a liposome carrier and a nucleic acid molecule according to claim 4.

17. A vaccine, comprising the pharmaceutical formulation according to claim 1; and
an adjuvant,
optionally, the adjuvant comprises at least one of a TLR agonist or Mn²⁺,
optionally, the TLR agonist comprises at least one of CpG, R837, MPLA, or derivatives thereof.

18. A recombinant cell, carrying a nucleic acid fragment or the nucleic acid molecule according to claim 4,
wherein the nucleic acid fragment comprises at least one of a first nucleic acid fragment, a second nucleic acid fragment, or a third nucleic acid fragment; wherein:
the first nucleic acid fragment encodes an M protein of feline infectious peritonitis virus;
the second nucleic acid fragment encodes an N protein of feline infectious peritonitis virus;
the third nucleic acid fragment encodes an S protein, S_ec protein, or SII protein of feline infectious peritonitis virus; and
the first nucleic acid fragment, the second nucleic acid fragment, and the third nucleic acid fragment are linked or not linked.

19. A method for constructing a feline infectious peritonitis virus vaccine, the method comprising:
introducing a nucleic acid fragment into a recipient cell,
wherein the nucleic acid fragment comprises at least one of a first nucleic acid fragment, a second nucleic acid fragment, and a third nucleic acid fragment, wherein:
the first nucleic acid fragment encodes an M protein of feline infectious peritonitis virus;
the second nucleic acid fragment encodes an N protein of feline infectious peritonitis virus;
the third nucleic acid fragment encodes an S protein, S_ec protein, or SII protein of feline infectious peritonitis virus; and
the first nucleic acid fragment, the second nucleic acid fragment, and the third nucleic acid fragment are linked or not linked,
the method further comprising, prior to said introducing into the recipient cell:
encapsulating the nucleic acid fragment with an encapsulation carrier,
wherein the encapsulation carrier is selected from at least one of a liposome, an exosome, a polymer carrier, a viral vector, or a nanoparticle, preferably a liposome,
wherein the recipient cell is a CRFK cell, HEK293FT cell, HEK293T cell, a BHK cell, or an insect cell, preferably a CRFK cell.

20. A method for preventing or treating a disease caused by feline infectious peritonitis virus infection, comprising administering to a subject the pharmaceutical formulation according to claim 1,
optionally, the subject is selected from a cat.


SEQUENCE LISTING (201 sequences)

SEQ ID NO: 1 - protein, 245 aa, Feline Infectious Peritonitis Virus
ERYCAMQNTGSQCINGTDSSCSTCFERGGLIWHLANWNFSWSVILIVFITVLKYGRPQFSWLVYGIKMLIMWLLWPIVLALTIFNAYSEYQVSRYVMFGFSVAGAVVTFALWMMYFVRSIQLYRRTKSWWSFNPETNAILCVNALGRSYVLPLDGTPTGVTLTLLSGNLYAEGFKMAGGLTIEHLPKYVMIATPSRTIVYTLVGKQLKATTATGWAYYVKSKAGDYSTEARTDNLSEHEKLLHMV

SEQ ID NO: 2 - protein, 375 aa, Feline Infectious Peritonitis Virus
ATQGQRVNWGDEPSKRRGRSNSRGRKNNTIPLSFFNPIQLEPGSKFWSVCPRDFVPKGIGNKDQQIGYWNRQERYRIVKGQRKELPERWFFYFLGTGPQADAKFKDKIDGVFWVAKDGAMNKPTTLGTRGTNNESKPLKFDGKIPPQFQLEVNRSRNNSRSGSQSRSVSRNRSQSRGRQQSNNQNNVEDTIVAVLQKLGVTEKQRSRSKSRDRGDSKPRDTTPNNANKHTWKKTAGKGDVTNFYGARSASANFGDSDLVANGNAAKSYPQIAECVPSVSSMLFGSQWSAEDDGDQVKVTLTHTYYLPKDDAKTSQFLEQIDAYKRPSQVAKDQRQRKSRSKSAEKKPEELSVTLVEAYTDVFDDTQVEMIDEVTN

SEQ ID NO: 3 - protein, 1433 aa, Feline Infectious Peritonitis Virus
TTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDNSGLHDLSVLHLDSCTDYNIYGRTGVGIIRRTNSTLLSGLYYTSLSGDLLGFKNVSDGVIYSVTPCDVSAQAAVIDGAIVGAMTSINSELLGLTHWTTTPNFYYYSIYNYTSERTRDTAIDSNDVDCEPVITYSNIGVCKNGALVFINVTHSDGDVQPISTGNVTIPTNFTISVQVEYMQVYTTPVSIDCARYVCNGNPRCNKLLTQYVSACQTIEQALAMGARLENMEVDSMLFVSENALKLASVEAFNSTENLDSIYKEWPSIGGSWLGGLKDILPSHNSKRKYGSAIEDLLFDKVVTSGLGTVDEDYKRCTGGYDIADLVCAQYYNGIMVLPGVANADKMTMYTASLAGGITLGALGGGAVAIPFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDPPSADAQVDRLITGRLTALNAFVSQTLTRQAEVRASRQLAKDKVNECVRSQSQRFGFCGNGTHLFSLANAAPNGMIFFHTVLLPTAYETVTAWSGICASDGDRTFGLVVKDVQLTLFRNLDDKFYLTPRTMYQPRVATSSDFVQIEGCDVLFVNATVIDLPSIIPDYIDINQTVQDILENYRPNWTVPEFTLDIFNATYLNLTGEIDDLEFRSEKLHNTTVELAILIDNINNTLVNLEWLNRIETYVKWPWYVWLLIGLVVVFCIPLLLFCCFSTGCCGCIGCLGSCCHSICSRRQFENYEPIEKVHVH

SEQ ID NO: 4 - DNA, 735 bp, Feline Infectious Peritonitis Virus
  source 1..735: mol_type = genomic DNA
gaacgctactgtgccatgcaaaatacaggctcgcagtgcattaatggcacagattcatcatgtagcacctgttttgaacgtggtggtcttatttggcatctggctaactggaacttcagctggtctgtaatattgattgtttttataacagtgttaaaatatggaagaccgcaattcagctggctcgtttatggcattaaaatgctgatcatgtggctattatggcctattgttctagcgcttacgatttttaatgcatactctgagtaccaagtttccagatatgtaatgttcggctttagtgttgcaggtgcagttgtaacgtttgcactatggatgatgtattttgtgagatctattcagctgtatagacggaccaaatcatggtggtcttttaatcctgaaaccaatgcgattctttgtgtcaatgcattgggtagaagctatgtactccctcttgatggcactcctacaggtgttactcttaccctactttcaggaaatctatacgctgaaggttttaaaatggctggtggtcttaccatcgagcatttgcctaaatatgtcatgattgctacgcctagtagaaccatcgtttacacattagttggaaaacaactaaaggcaactactgccactggatgggcttactatgtaaaatctaaagctggtgattactcaacagaagcacgtactgataatttgagtgaacatgaaaaattattacatatggtg

SEQ ID NO: 5 - DNA, 735 bp, Feline Infectious Peritonitis Virus
  source 1..735: mol_type = genomic DNA
gagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 6 - DNA, 735 bp, Feline Infectious Peritonitis Virus
  source 1..735: mol_type = genomic DNA
gagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 7 - DNA, 735 bp, Feline Infectious Peritonitis Virus
  source 1..735: mol_type = genomic DNA
gagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 8 - DNA, 1125 bp, Feline Infectious Peritonitis Virus
  source 1..1125: mol_type = genomic DNA
gccacacagggacaacgcgtcaactggggagatgaaccttccaaaagacgtggtcgttctaactctcgtggtcggaagaataacactatacctctttcattcttcaatcccatccaactcgaaccaggatcaaaattttggagcgtatgtccgagagattttgttcccaagggaataggtaacaaggatcaacaaattggttattggaatagacaagagcgttaccgcattgtcaaaggtcagcgtaaggaacttcctgagaggtggtttttctacttcttaggcacaggacctcaagctgatgctaaatttaaagacaagattgatggagtcttctgggttgcaaaggatggtgccatgaataaaccaacaacacttggcactcgtggtaccaacaatgaatccaaaccactgaaatttgatggtaagataccaccgcaatttcagcttgaagtgaaccgatctaggaacaactcaagaagtggttctcagtctagatctgtctctagaaacaggtctcaatccagaggaagacaacaatccaataatcagaataatgttgaggatacaattgtagctgtgcttcagaaattaggtgttactgaaaagcaaaggtcacgttctaaatctagagatcgtggtgactctaaacctagagacacaacacctaataatgccaacaaacacacctggaagaagactgcaggtaaaggtgatgtgacaaatttctatggtgctagaagtgcttcagctaactttggtgatagtgatctcgttgccaacggtaacgctgccaaatcctaccctcagatagctgaatgcgttccatcagtgtctagcatgctcttcggtagtcaatggtctgctgaagatgatggtgatcaagtgaaagtcacgctcactcatacctattacctgccaaaagatgatgccaaaaccagccaattcctagaacagattgacgcttacaagcggccatctcaagtggctaaagatcagaggcaaagaaaatctcgttctaagtctgctgagaagaagcctgaggaattgtctgtaactcttgtagaggcatatacggatgtgtttgatgacacacaggttgagatgattgatgaggttacgaac

SEQ ID NO: 9 - DNA, 1125 bp, Feline Infectious Peritonitis Virus
  source 1..1125: mol_type = genomic DNA
gccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 10 - DNA, 1125 bp, Feline Infectious Peritonitis Virus
  source 1..1125: mol_type = genomic DNA
gccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 11 - DNA, 1125 bp, Feline Infectious Peritonitis Virus
  source 1..1125: mol_type = genomic DNA
gcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 12 - DNA, 4302 bp, Feline Infectious Peritonitis Virus
  source 1..4302: mol_type = genomic DNA
acaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaacaattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattggttgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatttttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgataatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggctgttattgatggtgccatagttggagctatgacttccattaacagtgaactgttaggtctaacacattggacaacgacacctaatttttattactactctatatataattacacaagtgagaggactcgtggcactgcaattgacagtaacgatgttgattgtgaacctgtcataacctattctaatataggtgtttgtaaaaatggtgctttggtttttattaacgtcacacattctgacggagacgtgcaaccaattagcactggtaatgtcacgatacctacaaattttactatatctgtgcaagttgaatacatgcaggtttacactacaccagtatcaatagattgtgcaagatacgtttgtaatggtaaccctagatgtaacaaattgttaacacaatatgtgtctgcatgtcaaactattgaacaagcacttgcaatgggtgccagacttgaaaacatggaggttgattccatgttgtttgtctcggaaaatgcccttaaattggcatctgttgaggcgttcaatagtacagaaaatttagatcctatttacaaagaatggcctagcataggtggttcttggctaggaggtctaaaagatatactaccgtcccataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagctaatgctgacaagatgactatgtacacagcatcacttgcaggtggtataacattaggtgcacttggtggtggcgccgtggctataccttttgcagtagcagtacaggctagacttaattatgttgctctacaaactgatgtattgaataaaaaccaacagatcctggctaatgctttcaatcaagctattggtaacattacacaggcttttggtaaggttaatgatgctatacatcaaacatcacaaggtcttgccactgttgctaaagcgttggcaaaagtgcaagatgttgtcaacacacaagggcaagctttaagtcaccttacagtacaattgcaaaataattttcaagccattagtagttctattagtgatatttataacaggcttgacgaactgagtgctgatgcacaagttgataggctgattacaggtagacttacagcacttaatgcatttgtgtctcagactctaaccagacaagcagaggttagggctagtagacaacttgccaaagacaaggttaatgaatgtgttaggtctcagtctcagagattcggattctgtggtaatggtacacatttgttttcactagcaaatgcagcaccaaatggcatgattttctttcatacagtactattaccaacagcttatgaaactgtaacagcttggtcaggtatttgtgcttcagatggcgatcgcactttcggacttgtcgttaaagatgtgcagttgacgttgtttcgtaatctagatgacaagttctatttgacccccagaactatgtatcagcctagagttgcaactagttctgattttgttcaaattgaagggtgtgatgtgttgtttgtcaacgcgactgtaattgatttgcctagtattatacctgactatattgacattaatcaaactgttcaagacatattagaaaattacagaccaaactggactgtacctgaatttacacttgatattttcaacgcaacctatttaaatctgactggtgaaattgatgacttagagtttaggtcagaaaagctacataacactacagtagaacttgccattctcattgataacattaataatacattagtcaatcttgaatggctcaatagaattgaaacttatgtaaaatggccttggtatgtgtggctactgataggtttagtagtagtattttgcataccattactgctattttgctgttttagcacaggttgttgtggatgcataggttgtttaggaagttgttgtcactctatatgtagtagaagacaatttgaaaattatgaaccaattgaaaaagtgcatgtccactaa

SEQ ID NO: 13 - DNA, 4299 bp, Feline Infectious Peritonitis Virus
  source 1..4299: mol_type = genomic DNA
accaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 14 - DNA, 4299 bp, Feline Infectious Peritonitis Virus
  source 1..4299: mol_type = genomic DNA
accaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 15 - DNA, 4299 bp, Feline Infectious Peritonitis Virus
  source 1..4299: mol_type = genomic DNA
accacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 16 - protein, 26 aa, Feline Infectious Peritonitis Virus
MRFVMSPTVLLLLLGALAAPQTWAGS

SEQ ID NO: 17 - DNA, 78 bp, Feline Infectious Peritonitis Virus
  source 1..78: mol_type = genomic DNA
atgagatttgtgatgtcccccactgtcctgctgctgctcctgggagccctggcagccccccagacctgggcaggcagc

SEQ ID NO: 18 - DNA, 78 bp, Feline Infectious Peritonitis Virus
  source 1..78: mol_type = genomic DNA
atgagatttgtgatgagccccactgtgctgctgctgctgctgggagccctggcagccccccagacctgggctggctcc

SEQ ID NO: 19 - DNA, 78 bp, Feline Infectious Peritonitis Virus
  source 1..78: mol_type = genomic DNA
atgcgattcgtcatgtcaccaaccgttttactattattactaggagcattagcagcaccgcaaacatgggcaggaagt

SEQ ID NO: 20 - DNA, 78 bp, Feline Infectious Peritonitis Virus
  source 1..78: mol_type = genomic DNA
atgagattcgtgatgtcccctaccgtactactactcctacttggcgcactagcagctcctcaaacttgggccggatcc

SEQ ID NO: 21 - DNA, 78 bp, Feline Infectious Peritonitis Virus
  source 1..78: mol_type = genomic DNA
atgagatttgtgatgagccccactgtgctgctgctgctgctgggagccctggcagccccccagacctgggctggctca

SEQ ID NO: 22 - DNA, 78 bp, Feline Infectious Peritonitis Virus
  source 1..78: mol_type = genomic DNA
atgcgttttgtaatgtcacctactgtactactactactactcggagcactagcagcacctcagacttgggccggatca

SEQ ID NO: 23 - protein, 55 aa, Feline Infectious Peritonitis Virus
FLGIIAGVVVLVVTVVVGAVIWRKKCSGRKGPSYSHAARDDSTQGSDSSLMAPKV

SEQ ID NO: 24 - DNA, 165 bp, Feline Infectious Peritonitis Virus
  source 1..165: mol_type = genomic DNA
ttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtc

SEQ ID NO: 25 - DNA, 165 bp, Feline Infectious Peritonitis Virus
  source 1..165: mol_type = genomic DNA
ttcctgggcatcatcgcaggagtggtggtcctggtggtcacagtcgtggtgggagcagtgatctggaggaagaagtgctcaggaaggaagggcccatcctactcccacgccgccagggacgactcaacccagggctcagacagctccctgatggcccccaaggtg

SEQ ID NO: 26 - DNA, 165 bp, Feline Infectious Peritonitis Virus
  source 1..165: mol_type = genomic DNA
ttcctgggcatcatcgctggcgtggtggtcctggtggtgactgtggtggtcggagccgtcatctggaggaagaagtgcagcggcagaaagggcccaagctacagccacgccgccagagatgacagcacccagggcagcgacagcagcctcatggcccccaaggtc

SEQ ID NO: 27 - DNA, 165 bp, Feline Infectious Peritonitis Virus
  source 1..165: mol_type = genomic DNA
ttcctgggcatcatcgcaggagtggtggtgctggtggtgacagtggtggtgggagcagtgatctggagaaagaaatgcagcgggagaaagggccccagctacagccacgccgccagggacgacagcacccagggcagcgacagcagcctcatggcccccaaggtg

SEQ ID NO: 28 - DNA, 165 bp, Feline Infectious Peritonitis Virus
  source 1..165: mol_type = genomic DNA
ttcctgggcatcatcgccggcgtggtggtcctggtggtgaccgtggtggtcggagcagtgatctggaggaagaagtgctcaggcaggaagggcccatcctacagccacgccgccagagatgacagcacccaaggctcagacagctccctgatggcccccaaggtg

SEQ ID NO: 29 - DNA, 165 bp, Feline Infectious Peritonitis Virus
  source 1..165: mol_type = genomic DNA
ttccttggaatcatagctggggtcgttgtcctcgtagtgactgtagtggtaggcgcagttatctggaggaagaaatgctcggggaggaaagggccctcttacagccatgctgccagggatgactccacacaggggtcagatagcagcctcatggccccaaaggtc

SEQ ID NO: 30 - DNA, 165 bp, Feline Infectious Peritonitis Virus
  source 1..165: mol_type = genomic DNA
ttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 31 - DNA, 165 bp, Feline Infectious Peritonitis Virus
  source 1..165: mol_type = genomic DNA
ttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 32 - DNA, 165 bp, Feline Infectious Peritonitis Virus
  source 1..165: mol_type = genomic DNA
ttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 33 - protein, 27 aa, Feline Infectious Peritonitis Virus
YPYDVPDYAYPYDVPDYAYPYDVPDYA

SEQ ID NO: 34 - DNA, 81 bp, Feline Infectious Peritonitis Virus
  source 1..81: mol_type = genomic DNA
tacccctacgatgtgccagactacgcctacccctacgatgtgccagactacgcctacccctacgatgtgccagactacgcc

SEQ ID NO: 35 - DNA, 81 bp, Feline Infectious Peritonitis Virus
  source 1..81: mol_type = genomic DNA
tacccctacgacgtgcccgactacgcctacccctacgacgtgccagactacgcctacccctacgacgtgccagactacgcc

SEQ ID NO: 36 - DNA, 81 bp, Feline Infectious Peritonitis Virus
  source 1..81: mol_type = genomic DNA
tacccatacgatgttccagactacgcttacccatatgacgtgccagactatgcctacccctacgacgtgcccgactacgca

SEQ ID NO: 37 - protein, 6 aa, Feline Infectious Peritonitis Virus
HHHHHH

SEQ ID NO: 38 - DNA, 18 bp, Feline Infectious Peritonitis Virus
  source 1..18: mol_type = genomic DNA
caccaccaccaccaccac

SEQ ID NO: 39 - DNA, 18 bp, Feline Infectious Peritonitis Virus
  source 1..18: mol_type = genomic DNA
catcatcatcatcaccac

SEQ ID NO: 40 - protein, 24 aa, Feline Infectious Peritonitis Virus
DYKDDDDKDYKDDDDKDYKDDDDK

SEQ ID NO: 41 - DNA, 72 bp, Feline Infectious Peritonitis Virus
  source 1..72: mol_type = genomic DNA
gactacaaagatgatgatgacaaggactacaaagacgacgacgacaaagactacaaggacgatgacgacaag

SEQ ID NO: 42 - DNA, 72 bp, Feline Infectious Peritonitis Virus
  source 1..72: mol_type = genomic DNA
gactacaaggatgatgatgacaaggactacaaagacgacgacgacaaggactacaaggatgacgatgacaag

SEQ ID NO: 43 - DNA, 72 bp, Feline Infectious Peritonitis Virus
  source 1..72: mol_type = genomic DNA
gactataaagacgacgatgacaaagattataaagatgatgacgacaaggactacaaggatgatgatgacaag

SEQ ID NO: 44 - DNA, 68 bp, Feline Infectious Peritonitis Virus
  source 1..68: mol_type = genomic DNA
ggatagctaccagcttacacttgcttctgacacaactgtgttcgctatcaaccacacaaacagacgcc

SEQ ID NO: 45 - DNA, 94 bp, Feline Infectious Peritonitis Virus
  source 1..94: mol_type = genomic DNA
gtgaagccggggcgatcgtcctgagggcccggggcctcttgggctccgggcacccgaacttcctgatctttgaataaagtctgagtgggctgca

SEQ ID NO: 46 - protein, 26 aa, Feline Infectious Peritonitis Virus
RAKRGSGATNFSLLKQAGDVEENPGP

SEQ ID NO: 47 - DNA, 78 bp, Feline Infectious Peritonitis Virus
  source 1..78: mol_type = genomic DNA
agagccaagagaggcagcggagccaccaacttcagcctgctgaagcaggccggcgacgtggaggagaacccaggacct

SEQ ID NO: 48 - protein, 15 aa, Feline Infectious Peritonitis Virus
GGGGSGGGGSGGGGS

SEQ ID NO: 49 - DNA, 45 bp, Feline Infectious Peritonitis Virus
  source 1..45: mol_type = genomic DNA
ggaggaggaggaagcggaggaggaggaagcggaggaggaggaagc

SEQ ID NO: 50 - skipped

SEQ ID NO: 51 - DNA, 705 bp, Feline Infectious Peritonitis Virus
  source 1..705: mol_type = genomic DNA
gagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 52 - DNA, 705 bp, Feline Infectious Peritonitis Virus
  source 1..705: mol_type = genomic DNA
gagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 53 - DNA, 705 bp, Feline Infectious Peritonitis Virus
  source 1..705: mol_type = genomic DNA
gagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 54 - DNA, 1095 bp, Feline Infectious Peritonitis Virus
  source 1..1095: mol_type = genomic DNA
gccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 55 - DNA, 1095 bp, Feline Infectious Peritonitis Virus
  source 1..1095: mol_type = genomic DNA
gccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 56 - DNA, 1095 bp, Feline Infectious Peritonitis Virus
  source 1..1095: mol_type = genomic DNA
gcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggtctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 57 - DNA, 4122 bp, Feline Infectious Peritonitis Virus
  source 1..4122: mol_type = genomic DNA
accaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccc

SEQ ID NO: 58 - DNA, 4122 bp, Feline Infectious Peritonitis Virus
  source 1..4122: mol_type = genomic DNA
accaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccc

SEQ ID NO: 59 - DNA, 4122 bp, Feline Infectious Peritonitis Virus
  source 1..4122: mol_type = genomic DNA
accacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcct

SEQ ID NO: 60 - DNA, 1956 bp, Feline Infectious Peritonitis Virus
  source 1..1956: mol_type = genomic DNA
tgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 61 - DNA, 1956 bp, Feline Infectious Peritonitis Virus
  source 1..1956: mol_type = genomic DNA
tgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 62 - DNA, 1956 bp, Feline Infectious Peritonitis Virus
  source 1..1956: mol_type = genomic DNA
tgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 63 - DNA, 738 bp, Feline Infectious Peritonitis Virus
  source 1..738: mol_type = genomic DNA
gagagatactgtgccatgaaggacgacagcagcaacacctgcatcaacggcaccaacagcagctgccagacctgctttgagagaggggacctgatctggcacctggccaactggaacttcagctggagcgtgatcctgatcgtgttcatcaccgtgctgcagtatggaagaccccagttcagctggctggtgtacggcatcaagatgctgatcatgtggctgctgtggcccatcgtgctggccctgaccatcttcaacgcctacagcgagtaccaggtgtccagatacgtgatgtttggcttcagcatcgccggggccgtggtgaccttcgccctgtgcatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 64 - DNA, 1128 bp, Feline Infectious Peritonitis Virus
  source 1..1128: mol_type = genomic DNA
gctacacaaggacagagagttaactggggagatgaaccaagcaagagaagagacagaagcaacagcagaggaagaaaaaatggaaacatccccctgtcctacttcaaccccatcaccctggagagcggcagcaagttctggaacatctgccccagggactttgtgcccaagggcattggaaacaaggaccagcagatcggctactggaacagacaggtgcgctacagaattgtgcggggccagaggaaggagctgcccgagagatggttcttctacttctctggaacaggcccccacgctgatgccaagttcaaggacaagattgatggagtgttctgggtggccagagatggagccatgaacaagcccaccaccctgggcaccagaggcaccaacaacgagagcaagcccctgaagtttgatggcaagatccccccccagttccagctggaggtgaacagaagcagaaacaacagcagaagcggcagccagcccaggagcgtgtccagaagcagaagccagagcagaggaagacagcagagcaacaaccagaacaccaacgtggaggacaccatcgtggccgtgctgagcaagctgggcgtgacagacaagcagaggagcagaagcaagtctggagaaagaaaccagagcaagcccagagacaccacccccaagaatgccaacaagcacacctggaagaagacagctggcaagggggacgtgaccaacttctatggagccaggagcagcagcgccaactttggagacagcgacctggtggccaatggaaatgccgccaagtgctacccccagatcgccgagtgtgtgcccagcgccagcagcatcctgtttggcagccagtggagcgccgaggaggccggggaccaggtgaaggtgaccctgacccacacctactacctgcccaaggatgatgccaagaccagccagttcctggagcagattgatgcctacaagaggcccagcgaggtggccaaggaccagaggcagaggaagagcagaagcaagtctgctgacaagaagccagaggagctgtcggtgaccctcgtcgaggcatacaccgatgtcttcgacgacactcaggtggagatgatcgacgaggtcaccaac

SEQ ID NO: 65 - DNA, 1938 bp, Feline Infectious Peritonitis Virus
  source 1..1938: mol_type = genomic DNA
tgcagcagcgtgatcacctacagcagcttcgccatctgcaacacaggagagatcaagtacgtgaacgtgacccacgtggagactgtggacgacaacatcggggtgatcaagcccatcagcaccggcaacatcaccatccccaagaacttcacagtggccgtgcaggccgagtacatccagatccaggtgaagcctgtggtggtggactgcgccaagtacgtgtgcaatggaaatggccactgcctgaacctgctgacccagtacacctctgcctgccagaccatcgagaacgccctgaacctgggcgccagactggagtccctgatgctgtctgagatggtgaccgtgagcgagagaaacctggacctggccaccgtggagaagttcaacagcacagtgctgggcggggagaagctgggaggcttctactttgacggcctgaagagcctgctgcctcccaccatcggcaagagaagcgccgtggaggacctgctgttcaacaaggtggtgaccagcggcctggggaccgtggatgatgactacaagaagtgcagcgccggcacagatgtggccgacctggcctgtgcccagtactacaacggcatcatggtgctgcctggagtggtggaccagaacaagatggccatgtacaccgccagcctgattggaggcatggccctgggcagcatcaccagcgccgtggccgtgcccttcgccatgcaggtgcaggccagactgaactacgtggccctgcagacagatgtgctgcaggagaaccagaagatcctggccaacgccttcaacaacgccatcggcaacatcaccctggccctggggaaggtgagcaacagcatcaccaccatctctggaggcttccacaccatggccagcgccctgaccaagatccagagcgtggtgaaccagcagggggaggccctgtcccagctgaccagccagctgcagaagaacttccaggccatctcctcttccattgccgagatctacaacagactggagaaggctgaggccgacgcccaggtggacaggctgatcacaggaagactggccgccctgaacgcctacgtgtcccagaccctgacccagtatgccgaggtgaaggccagcagacagctggccatggagaaggtgaatgagtgtgtgaagagccagtctgacagatacggcttctgtggaaatggaacccacctgttcagcctggtgaactctgcccctgacggcctgctgttcttccacaccgtgctgctgcccacagagtgggaggaggtgacagcctggagtggcatctgtgtgaatgacacctacgcctacgtgctgaaagactttgactacagcatcttcagctacaacggcacctacatggtgacccccagaaacatgttccagcccagaaagccccagatgtcagacttcgtgcagatcaccagatgcgaggtgaccttcctgaacacaacctacaccaccttccaggagatcgtgatcgactacatcgacatcaacaagaccatcgccgacatgctggagcagtacaacctgaactacacaacccctgagctgaacctgcagctggagatcttcaaccagaccaagctgaacctgaccgccgagatcgaccagctggagcagagagccgacaacctgaccaacatcgcccacgagctgcagcagtacatcgacaacctgaacaagaccctggtggacctggagtggctgaacagaattgaaacctacgtgaagtggccctggtacgtgtggctgctgatcggcctggtgatcgtgttctgcatccctctgctgctgttctgctgcctgagcaccggctgctgtggctgcttcggcttcctgggctcctgctgccactccctgtgcagcaggaggcagtttgagtcctacgagcccatcgagaaggtgcacatccac

SEQ ID NO: 66 - DNA, 735 bp, Feline Infectious Peritonitis Virus
  source 1..735: mol_type = genomic DNA
gagagatactgtgccatgcaggacagcggcctgcagtgcatcaacggcaccaacagcagatgccagacctgctttgaaagaggggacctgatctggcacctggccaactggaacttctcctggagcgtgatcctgatcgtgttcatcaccgtgctgcagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccgtgcagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggacagacaacctgagcgagcacgagaagctgctgcacatggtg

SEQ ID NO: 67 - DNA, 1128 bp, Feline Infectious Peritonitis Virus
  source 1..1128: mol_type = genomic DNA
gctacacaaggacagagagttaactggggagatgaaccaagcaagagaagaggaagaagcaacagcagaggaagaaagaacaatgacatccccctgtccttctacaaccccatcaccctggagcagggcagcaagttctggaacctgtgccccagggacctggtgcccaagggcatcggcaacaaggaccagcagattggctactggaacagacagatcagatacagaattgtgaagggccagaggaaggagctggccgagaggtggttcttctacttcctgggcaccggcccccatgctgatgccaagttcaaggacaagattgatggagtgttctgggtggccagagatggggccatgaacaagcccaccaccctgggcaccaggggcaccaacaatgagagcaagcccctgaggtttgatggcaagatccccccccagttccagctggaggtgaacagaagcagaaacaacagcagaagcggcagccagagcagaagtgtgtccagaaacagaagccagagcagaggaagacaccacagcaacaaccagaacaacaatgtggaggacaccatcgtggccgtgctggagaagctgggggtgacagacaagcagaggagcagaagcaagcccagagaaagaagtgacagcaagcccagggacaccacccccaagaatgccaacaagcacacctggaagaagacagctggaaaaggagatgtgaccaccttctatggagccaggagcagcagcgccaactttggagacagtgacctggtggccaatggaaatgctgccaagtgctacccccagattgctgagtgtgtgccctctgtgagcagcatcatctttggcagccagtggtcagcagaggaggctggggaccaggtgaaggtgaccctgacccacacctactacctgcccaaggatgatgccaagaccagccagttcctggagcagattgatgcctacaagaggcccagcgaggtggccaaggaccagaggcagaggaggagcctgagcaagtctgctgacaagaagccagaggagctgtcggtgaccctcgtcgaggcgtacaccgacgtcttcgacgacactcaggtggagatgatcgacgaggtcaccaac

SEQ ID NO: 68 - DNA, 1956 bp, Feline Infectious Peritonitis Virus
  source 1..1956: mol_type = genomic DNA
tgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggaccccatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagaacctggccaatgccttcatccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaccattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagtactacgagcccatcgagaaggtgcacgtgcac

SEQ ID NO: 69 - DNA, 741 bp, Feline Infectious Peritonitis Virus
  source 1..741: mol_type = genomic DNA
gaccgctactgtgccatgcagcacgccagcagcaccagctgcatcaatggcaccagcaccaacagctgccagacctgctttgaaagaggagacttgatttggcacctggccaactggaacttcagctggagcgtcatcctcatcgtgttcatcaccgtgctgcagtatggaagaccccagctgagctggtttgtgtacggcatcaagatgctcatcatgtggctgctgtggcccatcgtgctggccctgaccatcttcaacgcctacagcgagtaccaggtgtccagatacgtgatgtttggcttctctgtggctggagctgtcatcacctttgccctgtggatgatgtactttgtgaggagcatccagctgtacaggaggaccaagagctggtggagcttcaacccagaaaccaatgccatcctgtgtgtgaatgccctgggcaggagctacgtgctgcccctggatggcatccccactggagtcaccctgaccctgctgtctggaaacctgtatgctgagtgcttcaagatggtgggcggcctgaccatcgagcacctgcccaagtacgtgatgattgccacccccagcagcaccatcgtgtacaccctggtgggcaagcagctgaaggccaccacagccaccggctgggcctactatgtgaagagcaaggctggagactacagcacagaggccaggacagacaacctgtctgaacatgagaagctgctgcacatggtg

SEQ ID NO: 70 - DNA, 1128 bp, Feline Infectious Peritonitis Virus
  source 1..1128: mol_type = genomic DNA
gccacccagggccagagggtcaactggggcgacgagcccagcaagaggagaggaagaagcaacagcagaggaaggaagaacaatgacatccccctgagcttctacaaccccatcaccctggagactggcagcaagttctggaatgtctgccccagggactttgtgcccaagggcatcggcaacaaggaccagcagattggctactggaacaagcaggcccgctacagaattgtgaagggccagaggaaggacctgcctgagaggtggttcttctacttcctgggcacaggcccccacgctgatgccaagttcaaggacaagattgatggagtcttctgggtggccaaggatggagccatgaacaagcccaccaccctgggcaccagaggcaccaacaatgagagcaagcccctgagatttgatgggaagatccccccccagttccagctggaggtgaaccagagcagaaacaacagcagaagcggcagccagagcagaagtgcctccagaaacagaagccagagcagaggaagacagcagagcaacaaccagaacaccaacgtggaggacaccatcgtggctgtgctgcagaagctgggcgtgacagacaagcagaggagcagaagcaagagcagagaaagaagcggcagcaacagcagggacaccacccccaagaatgccaacaagcacagctggaagaagacagctggcaagggagatgtgaccaacttctatggagccaggagtgccagcgccaacttcggggacagtgacctggtggccaatggaaatgccgccaagtgctacccccagattgctgagtgtgtgcccagcgtgtcctccatgctgtttggaagccagtggtcagcagaggatgctggggaccaggtgaaggtgaccctgacccacacctactacctgcccaaggatgatgccaagaccagccagttcctgggccagattgatgcctacaagaggcccagccaggtggtgaaggagcagaggcagaggaagagcagaagcaagtctgctgacaagaagccagaggagctgtctgtgaccctggtggaggcctacacagacgtgtttgatgacacccaggtggagatgattgatgaggtgaccaac

SEQ ID NO: 71 - DNA, 1956 bp, Feline Infectious Peritonitis Virus
  source 1..1956: mol_type = genomic DNA
tgtgagcccatcatcacctacttcaacatcggggtgtgcaagaatggggccctggtcttcatcaatgtgacccacagcgatggagatgtgcagcccatcagcacaggaaatgtgaccatccccaccaacttcaccatctctgtgcaggtggagtacatccaggtgtacaccacccctgtgtccatcgactgcagccgctacgtgtgcaatggaaaccccaggtgcaacaagctgctgacccagtacttctctgcctgccagaccatcgagcaggccctggccatgggagcccggctggagaacatggaggtggacagcatgctgtttgtgtctgaaaatgccctgaagctggcctctgtggaggccttcaacagcagtgagcacctggaccccatctacaaggagtggcccaacattggaggcagctggctggggggcctgaaggacatcctgcccagccacaacagcaagaggaactacagaagtgccatcgaggacctgctctttgacaaggtggtgacctctgggctgggcaccgtggatgatgactacaagaggtgcacaggaggctatgacattgcagacctggtgtgtgcccagtactaccatggcatcatggtgctgcctggagtggccaatgatgacaagatgaccatgtacacagcctccctggctggaggcatcaccctgggggccctgggcggaggagccgtggccatcccctttgctgtggctgtgcaggccaggctcaactacgtggccctgcagacagatgtgctcaacaagaaccagcagatcctggccaacgccttcaaccaggccattggaaacatcacccaggcctttgggaaggtgaatgatgccatccaccagaccagcaagggcctggccaccgtggccaaggccctggccaaggtgcaggatgtggtcaacacccagggccaggccctgagccacctcactgtccagctgcagaacaacttccaggccatcagcagcagcatctctgacatctacaacaggctggatgagctgtctgctgatgcccaggtggacagactcatcaccgggaggctgacagccctgaatgcctttgtcagccagaccctgaccaggcaggcagaggtgcgggcctcccggcagctggccaaggacaaggtgaatgagtgtgtgaggagccagagccagaggtttggcttctgtggaaatggcacccacctcttctccctggccaatgctgcccccaatggcatgatcttcttccacaccgtgctgctgcccaccgcctatgaaacagtgacagcctggagcggcatctgtgcctctgatggggaccacaccttcggcctggtggtgaaggatgtccagctgaccctcttcagaaacctggatgacaagttctacctgacccccaggaccatgtaccagccccgggtggccaccagcagcgactttgtgcagattgagggctgtgatgccctgtttgtgaatgccactgtcatcgagctgcccagcatcatcccagactacattgacatcaaccagaccgtgcaggacatcctgaagaactacaggcccaactggacagttcctgagctgaccctggacatcttcaacagcacctacctgaacctgacaggagaaatcaatgacctggagttcagaagtgagaagctgcacaacaccacagtggagctggctgtgctgatcgacaacatcaacaacaccctggtcaacctggagtggctgaacagaattgaaacctacgtgaagtggccctggtatgtttggctgctcatcggcctggtgctggtgttctgcatccccctgctcatgttctgctgcctgagcaccggctgctgcggctgcttcggctgcctgggcagctgctgccacagcctgttctccagaagacactttgagaactacgagcccatcgagaaggtgcacatccac

SEQ ID NO: 72 - DNA, 300 bp, Feline Infectious Peritonitis Virus
  source 1..300: mol_type = genomic DNA
ctggtcttcctgcatgctgtgctggtgactgtgctcatcctgcccctcatcggccgcatccagctgctggagagacttctcctgagccacctgctgaacctgaccacagtcagcaatgtcctgggggtcccagacagcagcctgcgggtcaactgcctgcagctgctgaagccagactgcctggacttcaacatcctgcacaaggtgctggcagaaacacggctgctggtggtggtgctgcgggtcatcttcctggtgctgctgggcttcagctgctacaccctgctgggggccctcttc

SEQ ID NO: 73 - DNA, 177 bp, Feline Infectious Peritonitis Virus
  source 1..177: mol_type = genomic DNA
gatgctgtgaagagcatcggcatctctgtggatgctgtgctggatgagctggacagcattgcctttgctgtcaccctgaaggtgctcttcaacagcgggaagctgctggtgtgcatcggcttcggggacacctttgaggaggctgagcagaaggcctatgccaagagcaagctggtg

SEQ ID NO: 74 - DNA, 819 bp, Feline Infectious Peritonitis Virus
  source 1..819: mol_type = genomic DNA
atgtacccctacgatgtgccagactacgcctacccctacgatgtgccagactacgcctacccctacgatgtgccagactacgccgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 75 - DNA, 819 bp, Feline Infectious Peritonitis Virus
  source 1..819: mol_type = genomic DNA
atgtacccctacgacgtgcccgactacgcctacccctacgacgtgccagactacgcctacccctacgacgtgccagactacgccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 76 - DNA, 819 bp, Feline Infectious Peritonitis Virus
  source 1..819: mol_type = genomic DNA
atgtacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 77 - DNA, 789 bp, Feline Infectious Peritonitis Virus
  source 1..789: mol_type = genomic DNA
atgtacccctacgatgtgccagactacgcctacccctacgatgtgccagactacgcctacccctacgatgtgccagactacgccgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 78 - DNA, 789 bp, Feline Infectious Peritonitis Virus
  source 1..789: mol_type = genomic DNA
atgtacccctacgacgtgcccgactacgcctacccctacgacgtgccagactacgcctacccctacgacgtgccagactacgccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 79 - DNA, 789 bp, Feline Infectious Peritonitis Virus
  source 1..789: mol_type = genomic DNA
atgtacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 80 - DNA, 894 bp, Feline Infectious Peritonitis Virus
  source 1..894: mol_type = genomic DNA
atgagatttgtgatgtcccccactgtcctgctgctgctcctgggagccctggcagccccccagacctgggcaggcagctacccctacgatgtgccagactacgcctacccctacgatgtgccagactacgcctacccctacgatgtgccagactacgccgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 81 - DNA, 894 bp, Feline Infectious Peritonitis Virus
  source 1..894: mol_type = genomic DNA
atgagatttgtgatgagccccactgtgctgctgctgctgctgggagccctggcagccccccagacctgggctggctcctacccctacgacgtgcccgactacgcctacccctacgacgtgccagactacgcctacccctacgacgtgccagactacgccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 82 - DNA, 894 bp, Feline Infectious Peritonitis Virus
  source 1..894: mol_type = genomic DNA
atgagattcgtgatgtcccctaccgtactactactcctacttggcgcactagcagctcctcaaacttgggccggatcctacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 83 - DNA, 1059 bp, Feline Infectious Peritonitis Virus
  source 1..1059: mol_type = genomic DNA
atgagatttgtgatgtcccccactgtcctgctgctgctcctgggagccctggcagccccccagacctgggcaggcagctacccctacgatgtgccagactacgcctacccctacgatgtgccagactacgcctacccctacgatgtgccagactacgccgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtcttcctgggcatcatcgctggcgtggtggtcctggtggtgactgtggtggtcggagccgtcatctggaggaagaagtgcagcggcagaaagggcccaagctacagccacgccgccagagatgacagcacccagggcagcgacagcagcctcatggcccccaaggtc

SEQ ID NO: 84 - DNA, 1059 bp, Feline Infectious Peritonitis Virus
  source 1..1059: mol_type = genomic DNA
atgagatttgtgatgagccccactgtgctgctgctgctgctgggagccctggcagccccccagacctgggctggctcctacccctacgacgtgcccgactacgcctacccctacgacgtgccagactacgcctacccctacgacgtgccagactacgccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtgttcctgggcatcatcgcaggagtggtggtcctggtggtcacagtcgtggtgggagcagtgatctggaggaagaagtgctcaggaaggaagggcccatcctactcccacgccgccagggacgactcaacccagggctcagacagctccctgatggcccccaaggtg

SEQ ID NO: 85 - DNA, 1059 bp, Feline Infectious Peritonitis Virus
  source 1..1059: mol_type = genomic DNA
atgagattcgtgatgtcccctaccgtactactactcctacttggcgcactagcagctcctcaaacttgggccggatcctacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtc

SEQ ID NO: 86 - DNA, 1146 bp, Feline Infectious Peritonitis Virus
  source 1..1146: mol_type = genomic DNA
atgcaccaccaccaccaccacgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 87 - DNA, 1146 bp, Feline Infectious Peritonitis Virus
  source 1..1146: mol_type = genomic DNA
atgcaccaccaccaccaccacgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 88 - DNA, 1146 bp, Feline Infectious Peritonitis Virus
  source 1..1146: mol_type = genomic DNA
atgcatcatcatcatcaccacgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 89 - DNA, 1116 bp, Feline Infectious Peritonitis Virus
  source 1..1116: mol_type = genomic DNA
atgcaccaccaccaccaccacgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 90 - DNA, 1116 bp, Feline Infectious Peritonitis Virus
  source 1..1116: mol_type = genomic DNA
atgcaccaccaccaccaccacgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 91 - DNA, 1116 bp, Feline Infectious Peritonitis Virus
  source 1..1116: mol_type = genomic DNA
atgcatcatcatcatcaccacgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggtctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 92 - DNA, 1221 bp, Feline Infectious Peritonitis Virus
  source 1..1221: mol_type = genomic DNA
atgagatttgtgatgtcccccactgtcctgctgctgctcctgggagccctggcagccccccagacctgggcaggcagccaccaccaccaccaccacgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 93 - DNA, 1221 bp, Feline Infectious Peritonitis Virus
  source 1..1221: mol_type = genomic DNA
atgagatttgtgatgagccccactgtgctgctgctgctgctgggagccctggcagccccccagacctgggctggctcccaccaccaccaccaccacgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 94 - DNA, 1221 bp, Feline Infectious Peritonitis Virus
  source 1..1221: mol_type = genomic DNA
atgcgattcgtcatgtcaccaaccgttttactattattactaggagcattagcagcaccgcaaacatgggcaggaagtcatcatcatcatcaccacgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 95 - DNA, 1386 bp, Feline Infectious Peritonitis Virus
  source 1..1386: mol_type = genomic DNA
atgagatttgtgatgtcccccactgtcctgctgctgctcctgggagccctggcagccccccagacctgggcaggcagccaccaccaccaccaccacgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaacttcctgggcatcatcgcaggagtggtggtgctggtggtgacagtggtggtgggagcagtgatctggagaaagaaatgcagcgggagaaagggccccagctacagccacgccgccagggacgacagcacccagggcagcgacagcagcctcatggcccccaaggtg

SEQ ID NO: 96 - DNA, 1386 bp, Feline Infectious Peritonitis Virus
  source 1..1386: mol_type = genomic DNA
atgagatttgtgatgagccccactgtgctgctgctgctgctgggagccctggcagccccccagacctgggctggctcccaccaccaccaccaccacgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaacttcctgggcatcatcgccggcgtggtggtcctggtggtgaccgtggtggtcggagcagtgatctggaggaagaagtgctcaggcaggaagggcccatcctacagccacgccgccagagatgacagcacccaaggctcagacagctccctgatggcccccaaggtg

SEQ ID NO: 97 - DNA, 1386 bp, Feline Infectious Peritonitis Virus
  source 1..1386: mol_type = genomic DNA
atgcgattcgtcatgtcaccaaccgttttactattattactaggagcattagcagcaccgcaaacatgggcaggaagtcatcatcatcatcaccacgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaacttccttggaatcatagctggggtcgttgtcctcgtagtgactgtagtggtaggcgcagttatctggaggaagaaatgctcggggaggaaagggccctcttacagccatgctgccagggatgactccacacaggggtcagatagcagcctcatggccccaaaggtc

SEQ ID NO: 98 - DNA, 4374 bp, Feline Infectious Peritonitis Virus
  source 1..4374: mol_type = genomic DNA
atggactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 99 - DNA, 4374 bp, Feline Infectious Peritonitis Virus
  source 1..4374: mol_type = genomic DNA
atggactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 100 - DNA, 4374 bp, Feline Infectious Peritonitis Virus
  source 1..4374: mol_type = genomic DNA
atggactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 101 - DNA, 4197 bp, Feline Infectious Peritonitis Virus
  source 1..4197: mol_type = genomic DNA
atggactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccc

SEQ ID NO: 102 - DNA, 4197 bp, Feline Infectious Peritonitis Virus
  source 1..4197: mol_type = genomic DNA
atggactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccc

SEQ ID NO: 103 - DNA, 4197 bp, Feline Infectious Peritonitis Virus
  source 1..4197: mol_type = genomic DNA
atggactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcct

SEQ ID NO: 104 - DNA, 2031 bp, Feline Infectious Peritonitis Virus
  source 1..2031: mol_type = genomic DNA
atggactacaaggatgatgatgacaaggactacaaagacgacgacgacaaggactacaaggatgacgatgacaagtgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 105 - DNA, 2031 bp, Feline Infectious Peritonitis Virus
  source 1..2031: mol_type = genomic DNA
atggactacaaagatgatgatgacaaggactacaaagacgacgacgacaaagactacaaggacgatgacgacaagtgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 106 - DNA, 2031 bp, Feline Infectious Peritonitis Virus
  source 1..2031: mol_type = genomic DNA
atggactataaagacgacgatgacaaagattataaagatgatgacgacaaggactacaaggatgatgatgacaagtgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 107 - DNA, 4449 bp, Feline Infectious Peritonitis Virus
  source 1..4449: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 108 - DNA, 4449 bp, Feline Infectious Peritonitis Virus
  source 1..4449: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 109 - DNA, 4449 bp, Feline Infectious Peritonitis Virus
  source 1..4449: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 110 - DNA, 4272 bp, Feline Infectious Peritonitis Virus
  source 1..4272: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccc

SEQ ID NO: 111 - DNA, 4272 bp, Feline Infectious Peritonitis Virus
  source 1..4272: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccc

SEQ ID NO: 112 - DNA, 4272 bp, Feline Infectious Peritonitis Virus
  source 1..4272: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcct

SEQ ID NO: 113 - DNA, 2106 bp, Feline Infectious Peritonitis Virus
  source 1..2106: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagtgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 114 - DNA, 2106 bp, Feline Infectious Peritonitis Virus
  source 1..2106: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagtgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 115 - DNA, 2106 bp, Feline Infectious Peritonitis Virus
  source 1..2106: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagtgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 116 - DNA, 4614 bp, Feline Infectious Peritonitis Virus
  source 1..4614: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 117 - DNA, 4614 bp, Feline Infectious Peritonitis Virus
  source 1..4614: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccacttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 118 - DNA, 4614 bp, Feline Infectious Peritonitis Virus
  source 1..4614: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacatttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 119 - DNA, 4437 bp, Feline Infectious Peritonitis Virus
  source 1..4437: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggcccttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 120 - DNA, 4437 bp, Feline Infectious Peritonitis Virus
  source 1..4437: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggcccttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 121 - DNA, 4437 bp, Feline Infectious Peritonitis Virus
  source 1..4437: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcctttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 122 - DNA, 2271 bp, Feline Infectious Peritonitis Virus
  source 1..2271: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagtgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 123 - DNA, 2271 bp, Feline Infectious Peritonitis Virus
  source 1..2271: mol_type = genomic DNA
atgagatttgtgatgagccccactgtgctgctgctgctgctgggagccctggcagccccccagacctgggctggctcagactacaaagatgatgatgacaaggactacaaagacgacgacgacaaagactacaaggacgatgacgacaagtgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccacttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 124 - DNA, 2271 bp, Feline Infectious Peritonitis Virus
  source 1..2271: mol_type = genomic DNA
atgcgttttgtaatgtcacctactgtactactactactactcggagcactagcagcacctcagacttgggccggatcagactataaagacgacgatgacaaagattataaagatgatgacgacaaggactacaaggatgatgatgacaagtgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacatttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 125 - DNA, 321 bp, Feline Infectious Peritonitis Virus
  source 1..321: mol_type = genomic DNA
atgcaccaccaccaccaccacctggtcttcctgcatgctgtgctggtgactgtgctcatcctgcccctcatcggccgcatccagctgctggagagacttctcctgagccacctgctgaacctgaccacagtcagcaatgtcctgggggtcccagacagcagcctgcgggtcaactgcctgcagctgctgaagccagactgcctggacttcaacatcctgcacaaggtgctggcagaaacacggctgctggtggtggtgctgcgggtcatcttcctggtgctgctgggcttcagctgctacaccctgctgggggccctcttc

SEQ ID NO: 126 - DNA, 252 bp, Feline Infectious Peritonitis Virus
  source 1..252: mol_type = genomic DNA
atggactacaaggatgatgatgacaaggactacaaagacgacgacgacaaggactacaaggatgacgatgacaaggatgctgtgaagagcatcggcatctctgtggatgctgtgctggatgagctggacagcattgcctttgctgtcaccctgaaggtgctcttcaacagcgggaagctgctggtgtgcatcggcttcggggacacctttgaggaggctgagcagaaggcctatgccaagagcaagctggtg

SEQ ID NO: 127 - DNA, 1062 bp, Feline Infectious Peritonitis Virus
  source 1..1062: mol_type = genomic DNA
atgagatttgtgatgtcccccactgtcctgctgctgctcctgggagccctggcagccccccagacctgggcaggcagctacccatacgatgttccagactacgcttacccatatgacgtgccagactatgcctacccctacgacgtgcccgactacgcagagagatactgtgccatgaaggacgacagcagcaacacctgcatcaacggcaccaacagcagctgccagacctgctttgagagaggggacctgatctggcacctggccaactggaacttcagctggagcgtgatcctgatcgtgttcatcaccgtgctgcagtatggaagaccccagttcagctggctggtgtacggcatcaagatgctgatcatgtggctgctgtggcccatcgtgctggccctgaccatcttcaacgcctacagcgagtaccaggtgtccagatacgtgatgtttggcttcagcatcgccggggccgtggtgaccttcgccctgtgcatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 128 - DNA, 1149 bp, Feline Infectious Peritonitis Virus
  source 1..1149: mol_type = genomic DNA
atgcaccaccaccaccaccacgctacacaaggacagagagttaactggggagatgaaccaagcaagagaagagacagaagcaacagcagaggaagaaaaaatggaaacatccccctgtcctacttcaaccccatcaccctggagagcggcagcaagttctggaacatctgccccagggactttgtgcccaagggcattggaaacaaggaccagcagatcggctactggaacagacaggtgcgctacagaattgtgcggggccagaggaaggagctgcccgagagatggttcttctacttctctggaacaggcccccacgctgatgccaagttcaaggacaagattgatggagtgttctgggtggccagagatggagccatgaacaagcccaccaccctgggcaccagaggcaccaacaacgagagcaagcccctgaagtttgatggcaagatccccccccagttccagctggaggtgaacagaagcagaaacaacagcagaagcggcagccagcccaggagcgtgtccagaagcagaagccagagcagaggaagacagcagagcaacaaccagaacaccaacgtggaggacaccatcgtggccgtgctgagcaagctgggcgtgacagacaagcagaggagcagaagcaagtctggagaaagaaaccagagcaagcccagagacaccacccccaagaatgccaacaagcacacctggaagaagacagctggcaagggggacgtgaccaacttctatggagccaggagcagcagcgccaactttggagacagcgacctggtggccaatggaaatgccgccaagtgctacccccagatcgccgagtgtgtgcccagcgccagcagcatcctgtttggcagccagtggagcgccgaggaggccggggaccaggtgaaggtgaccctgacccacacctactacctgcccaaggatgatgccaagaccagccagttcctggagcagattgatgcctacaagaggcccagcgaggtggccaaggaccagaggcagaggaagagcagaagcaagtctgctgacaagaagccagaggagctgtcggtgaccctcgtcgaggcatacaccgatgtcttcgacgacactcaggtggagatgatcgacgaggtcaccaac

SEQ ID NO: 129 - DNA, 2253 bp, Feline Infectious Peritonitis Virus
  source 1..2253: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagtgcagcagcgtgatcacctacagcagcttcgccatctgcaacacaggagagatcaagtacgtgaacgtgacccacgtggagactgtggacgacaacatcggggtgatcaagcccatcagcaccggcaacatcaccatccccaagaacttcacagtggccgtgcaggccgagtacatccagatccaggtgaagcctgtggtggtggactgcgccaagtacgtgtgcaatggaaatggccactgcctgaacctgctgacccagtacacctctgcctgccagaccatcgagaacgccctgaacctgggcgccagactggagtccctgatgctgtctgagatggtgaccgtgagcgagagaaacctggacctggccaccgtggagaagttcaacagcacagtgctgggcggggagaagctgggaggcttctactttgacggcctgaagagcctgctgcctcccaccatcggcaagagaagcgccgtggaggacctgctgttcaacaaggtggtgaccagcggcctggggaccgtggatgatgactacaagaagtgcagcgccggcacagatgtggccgacctggcctgtgcccagtactacaacggcatcatggtgctgcctggagtggtggaccagaacaagatggccatgtacaccgccagcctgattggaggcatggccctgggcagcatcaccagcgccgtggccgtgcccttcgccatgcaggtgcaggccagactgaactacgtggccctgcagacagatgtgctgcaggagaaccagaagatcctggccaacgccttcaacaacgccatcggcaacatcaccctggccctggggaaggtgagcaacagcatcaccaccatctctggaggcttccacaccatggccagcgccctgaccaagatccagagcgtggtgaaccagcagggggaggccctgtcccagctgaccagccagctgcagaagaacttccaggccatctcctcttccattgccgagatctacaacagactggagaaggctgaggccgacgcccaggtggacaggctgatcacaggaagactggccgccctgaacgcctacgtgtcccagaccctgacccagtatgccgaggtgaaggccagcagacagctggccatggagaaggtgaatgagtgtgtgaagagccagtctgacagatacggcttctgtggaaatggaacccacctgttcagcctggtgaactctgcccctgacggcctgctgttcttccacaccgtgctgctgcccacagagtgggaggaggtgacagcctggagtggcatctgtgtgaatgacacctacgcctacgtgctgaaagactttgactacagcatcttcagctacaacggcacctacatggtgacccccagaaacatgttccagcccagaaagccccagatgtcagacttcgtgcagatcaccagatgcgaggtgaccttcctgaacacaacctacaccaccttccaggagatcgtgatcgactacatcgacatcaacaagaccatcgccgacatgctggagcagtacaacctgaactacacaacccctgagctgaacctgcagctggagatcttcaaccagaccaagctgaacctgaccgccgagatcgaccagctggagcagagagccgacaacctgaccaacatcgcccacgagctgcagcagtacatcgacaacctgaacaagaccctggtggacctggagtggctgaacagaattgaaacctacgtgaagtggccctggtacgtgtggctgctgatcggcctggtgatcgtgttctgcatccctctgctgctgttctgctgcctgagcaccggctgctgtggctgcttcggcttcctgggctcctgctgccactccctgtgcagcaggaggcagtttgagtcctacgagcccatcgagaaggtgcacatccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 130 - DNA, 1059 bp, Feline Infectious Peritonitis Virus
  source 1..1059: mol_type = genomic DNA
atgagatttgtgatgtcccccactgtcctgctgctgctcctgggagccctggcagccccccagacctgggcaggcagctacccatacgatgttccagactacgcttacccatatgacgtgccagactatgcctacccctacgacgtgcccgactacgcagagagatactgtgccatgcaggacagcggcctgcagtgcatcaacggcaccaacagcagatgccagacctgctttgaaagaggggacctgatctggcacctggccaactggaacttctcctggagcgtgatcctgatcgtgttcatcaccgtgctgcagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccgtgcagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggacagacaacctgagcgagcacgagaagctgctgcacatggtgttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 131 - DNA, 1149 bp, Feline Infectious Peritonitis Virus
  source 1..1149: mol_type = genomic DNA
atgcaccaccaccaccaccacgctacacaaggacagagagttaactggggagatgaaccaagcaagagaagaggaagaagcaacagcagaggaagaaagaacaatgacatccccctgtccttctacaaccccatcaccctggagcagggcagcaagttctggaacctgtgccccagggacctggtgcccaagggcatcggcaacaaggaccagcagattggctactggaacagacagatcagatacagaattgtgaagggccagaggaaggagctggccgagaggtggttcttctacttcctgggcaccggcccccatgctgatgccaagttcaaggacaagattgatggagtgttctgggtggccagagatggggccatgaacaagcccaccaccctgggcaccaggggcaccaacaatgagagcaagcccctgaggtttgatggcaagatccccccccagttccagctggaggtgaacagaagcagaaacaacagcagaagcggcagccagagcagaagtgtgtccagaaacagaagccagagcagaggaagacaccacagcaacaaccagaacaacaatgtggaggacaccatcgtggccgtgctggagaagctgggggtgacagacaagcagaggagcagaagcaagcccagagaaagaagtgacagcaagcccagggacaccacccccaagaatgccaacaagcacacctggaagaagacagctggaaaaggagatgtgaccaccttctatggagccaggagcagcagcgccaactttggagacagtgacctggtggccaatggaaatgctgccaagtgctacccccagattgctgagtgtgtgccctctgtgagcagcatcatctttggcagccagtggtcagcagaggaggctggggaccaggtgaaggtgaccctgacccacacctactacctgcccaaggatgatgccaagaccagccagttcctggagcagattgatgcctacaagaggcccagcgaggtggccaaggaccagaggcagaggaggagcctgagcaagtctgctgacaagaagccagaggagctgtcggtgaccctcgtcgaggcgtacaccgacgtcttcgacgacactcaggtggagatgatcgacgaggtcaccaac

SEQ ID NO: 132 - DNA, 2271 bp, Feline Infectious Peritonitis Virus
  source 1..2271: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagtgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggaccccatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagaacctggccaatgccttcatccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaccattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagtactacgagcccatcgagaaggtgcacgtgcacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 133 - DNA, 1065 bp, Feline Infectious Peritonitis Virus
  source 1..1065: mol_type = genomic DNA
atgagatttgtgatgtcccccactgtcctgctgctgctcctgggagccctggcagccccccagacctgggcaggcagctacccatacgatgttccagactacgcttacccatatgacgtgccagactatgcctacccctacgacgtgcccgactacgcagaccgctactgtgccatgcagcacgccagcagcaccagctgcatcaatggcaccagcaccaacagctgccagacctgctttgaaagaggagacttgatttggcacctggccaactggaacttcagctggagcgtcatcctcatcgtgttcatcaccgtgctgcagtatggaagaccccagctgagctggtttgtgtacggcatcaagatgctcatcatgtggctgctgtggcccatcgtgctggccctgaccatcttcaacgcctacagcgagtaccaggtgtccagatacgtgatgtttggcttctctgtggctggagctgtcatcacctttgccctgtggatgatgtactttgtgaggagcatccagctgtacaggaggaccaagagctggtggagcttcaacccagaaaccaatgccatcctgtgtgtgaatgccctgggcaggagctacgtgctgcccctggatggcatccccactggagtcaccctgaccctgctgtctggaaacctgtatgctgagtgcttcaagatggtgggcggcctgaccatcgagcacctgcccaagtacgtgatgattgccacccccagcagcaccatcgtgtacaccctggtgggcaagcagctgaaggccaccacagccaccggctgggcctactatgtgaagagcaaggctggagactacagcacagaggccaggacagacaacctgtctgaacatgagaagctgctgcacatggtgttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 134 - DNA, 1149 bp, Feline Infectious Peritonitis Virus
  source 1..1149: mol_type = genomic DNA
atgcaccaccaccaccaccacgccacccagggccagagggtcaactggggcgacgagcccagcaagaggagaggaagaagcaacagcagaggaaggaagaacaatgacatccccctgagcttctacaaccccatcaccctggagactggcagcaagttctggaatgtctgccccagggactttgtgcccaagggcatcggcaacaaggaccagcagattggctactggaacaagcaggcccgctacagaattgtgaagggccagaggaaggacctgcctgagaggtggttcttctacttcctgggcacaggcccccacgctgatgccaagttcaaggacaagattgatggagtcttctgggtggccaaggatggagccatgaacaagcccaccaccctgggcaccagaggcaccaacaatgagagcaagcccctgagatttgatgggaagatccccccccagttccagctggaggtgaaccagagcagaaacaacagcagaagcggcagccagagcagaagtgcctccagaaacagaagccagagcagaggaagacagcagagcaacaaccagaacaccaacgtggaggacaccatcgtggctgtgctgcagaagctgggcgtgacagacaagcagaggagcagaagcaagagcagagaaagaagcggcagcaacagcagggacaccacccccaagaatgccaacaagcacagctggaagaagacagctggcaagggagatgtgaccaacttctatggagccaggagtgccagcgccaacttcggggacagtgacctggtggccaatggaaatgccgccaagtgctacccccagattgctgagtgtgtgcccagcgtgtcctccatgctgtttggaagccagtggtcagcagaggatgctggggaccaggtgaaggtgaccctgacccacacctactacctgcccaaggatgatgccaagaccagccagttcctgggccagattgatgcctacaagaggcccagccaggtggtgaaggagcagaggcagaggaagagcagaagcaagtctgctgacaagaagccagaggagctgtctgtgaccctggtggaggcctacacagacgtgtttgatgacacccaggtggagatgattgatgaggtgaccaac

SEQ ID NO: 135 - DNA, 2271 bp, Feline Infectious Peritonitis Virus
  source 1..2271: mol_type = genomic DNA
atgagatttgtgatgtcccccactgtcctgctgctgctcctgggagccctggcagccccccagacctgggcaggcagcgactacaaggatgatgatgacaaggactacaaagacgacgacgacaaggactacaaggatgacgatgacaagtgtgagcccatcatcacctacttcaacatcggggtgtgcaagaatggggccctggtcttcatcaatgtgacccacagcgatggagatgtgcagcccatcagcacaggaaatgtgaccatccccaccaacttcaccatctctgtgcaggtggagtacatccaggtgtacaccacccctgtgtccatcgactgcagccgctacgtgtgcaatggaaaccccaggtgcaacaagctgctgacccagtacttctctgcctgccagaccatcgagcaggccctggccatgggagcccggctggagaacatggaggtggacagcatgctgtttgtgtctgaaaatgccctgaagctggcctctgtggaggccttcaacagcagtgagcacctggaccccatctacaaggagtggcccaacattggaggcagctggctggggggcctgaaggacatcctgcccagccacaacagcaagaggaactacagaagtgccatcgaggacctgctctttgacaaggtggtgacctctgggctgggcaccgtggatgatgactacaagaggtgcacaggaggctatgacattgcagacctggtgtgtgcccagtactaccatggcatcatggtgctgcctggagtggccaatgatgacaagatgaccatgtacacagcctccctggctggaggcatcaccctgggggccctgggcggaggagccgtggccatcccctttgctgtggctgtgcaggccaggctcaactacgtggccctgcagacagatgtgctcaacaagaaccagcagatcctggccaacgccttcaaccaggccattggaaacatcacccaggcctttgggaaggtgaatgatgccatccaccagaccagcaagggcctggccaccgtggccaaggccctggccaaggtgcaggatgtggtcaacacccagggccaggccctgagccacctcactgtccagctgcagaacaacttccaggccatcagcagcagcatctctgacatctacaacaggctggatgagctgtctgctgatgcccaggtggacagactcatcaccgggaggctgacagccctgaatgcctttgtcagccagaccctgaccaggcaggcagaggtgcgggcctcccggcagctggccaaggacaaggtgaatgagtgtgtgaggagccagagccagaggtttggcttctgtggaaatggcacccacctcttctccctggccaatgctgcccccaatggcatgatcttcttccacaccgtgctgctgcccaccgcctatgaaacagtgacagcctggagcggcatctgtgcctctgatggggaccacaccttcggcctggtggtgaaggatgtccagctgaccctcttcagaaacctggatgacaagttctacctgacccccaggaccatgtaccagccccgggtggccaccagcagcgactttgtgcagattgagggctgtgatgccctgtttgtgaatgccactgtcatcgagctgcccagcatcatcccagactacattgacatcaaccagaccgtgcaggacatcctgaagaactacaggcccaactggacagttcctgagctgaccctggacatcttcaacagcacctacctgaacctgacaggagaaatcaatgacctggagttcagaagtgagaagctgcacaacaccacagtggagctggctgtgctgatcgacaacatcaacaacaccctggtcaacctggagtggctgaacagaattgaaacctacgtgaagtggccctggtatgtttggctgctcatcggcctggtgctggtgttctgcatccccctgctcatgttctgctgcctgagcaccggctgctgcggctgcttcggctgcctgggcagctgctgccacagcctgttctccagaagacactttgagaactacgagcccatcgagaaggtgcacatccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 136 - DNA, 738 bp, Feline Infectious Peritonitis Virus
  source 1..738: mol_type = genomic DNA
atggagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 137 - DNA, 738 bp, Feline Infectious Peritonitis Virus
  source 1..738: mol_type = genomic DNA
atggagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 138 - DNA, 738 bp, Feline Infectious Peritonitis Virus
  source 1..738: mol_type = genomic DNA
atggagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 139 - DNA, 708 bp, Feline Infectious Peritonitis Virus
  source 1..708: mol_type = genomic DNA
atggagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 140 - DNA, 708 bp, Feline Infectious Peritonitis Virus
  source 1..708: mol_type = genomic DNA
atggagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 141 - DNA, 708 bp, Feline Infectious Peritonitis Virus
  source 1..708: mol_type = genomic DNA
atggagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 142 - DNA, 813 bp, Feline Infectious Peritonitis Virus
  source 1..813: mol_type = genomic DNA
atgagatttgtgatgtcccccactgtcctgctgctgctcctgggagccctggcagccccccagacctgggcaggcagcgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtc

SEQ ID NO: 143 - DNA, 813 bp, Feline Infectious Peritonitis Virus
  source 1..813: mol_type = genomic DNA
atgagatttgtgatgagccccactgtgctgctgctgctgctgggagccctggcagccccccagacctgggctggctccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtg

SEQ ID NO: 144 - DNA, 813 bp, Feline Infectious Peritonitis Virus
  source 1..813: mol_type = genomic DNA
atgagattcgtgatgtcccctaccgtactactactcctacttggcgcactagcagctcctcaaacttgggccggatccgagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtc

SEQ ID NO: 145 - DNA, 978 bp, Feline Infectious Peritonitis Virus
  source 1..978: mol_type = genomic DNA
atgagatttgtgatgtcccccactgtcctgctgctgctcctgggagccctggcagccccccagacctgggcaggcagcgagagatactgtgccatgcagaacacaggcagccagtgcatcaatggaacagacagcagctgcagcacctgctttgaaagaggaggcctgatctggcacctggccaactggaacttcagctggagtgtgatcctgattgtcttcattacagtgctgaagtacggcaggccccagttcagctggctggtgtacggcatcaagatgctcatcatgtggctgctgtggcccattgtgttggccctcaccatcttcaatgcctacagcgagtaccaggtgtccagatacgtgatgttcggcttctcagtggcaggagccgtggtgacctttgccctctggatgatgtactttgtgaggtccatccagctctacagaagaacaaagagctggtggagcttcaacccagaaaccaacgccatcctgtgtgtcaatgccctgggcagatcctatgtgctgcccctggatggcacccccacaggcgtcaccctcaccctcctgagcggcaacctgtacgccgagggcttcaagatggccggcggcctgacaatcgagcacctgcccaagtatgtgatgattgccacccccagcaggacaatagtctacaccctggtgggaaaacagctgaaggctaccacagccacaggctgggcctactacgtcaagagcaaggccggcgactacagcacagaggccaggaccgacaacctgtccgaacatgaaaaactcctgcacatggtcttcctgggcatcatcgctggcgtggtggtcctggtggtgactgtggtggtcggagccgtcatctggaggaagaagtgcagcggcagaaagggcccaagctacagccacgccgccagagatgacagcacccagggcagcgacagcagcctcatggcccccaaggtc

SEQ ID NO: 146 - DNA, 978 bp, Feline Infectious Peritonitis Virus
  source 1..978: mol_type = genomic DNA
atgagatttgtgatgagccccactgtgctgctgctgctgctgggagccctggcagccccccagacctgggctggctccgagaggtactgcgccatgcagaacacaggctcccagtgcatcaacggaacagactcctcctgcagcacctgcttcgagagaggaggcctcatttggcacctggccaactggaacttcagctggtcagtcattctgatagtcttcataacagtgctgaagtacggcaggccccagttctcctggctcgtgtatggcatcaagatgctgatcatgtggctgctgtggcccatcgtcctggccctgaccatcttcaacgcctactcagagtaccaggtcagcaggtacgtgatgttcggcttctccgtggccggagcagtggtgaccttcgccctgtggatgatgtacttcgtgaggagcatccaactgtacaggaggaccaaaagctggtggtccttcaacccagaaaccaacgccatcctctgcgtgaacgccctgggcaggtcctacgtcctccccctggacggcacccccaccggggtcaccctcaccctcctgtcagggaacctgtacgctgagggcttcaagatggctggaggcctgacaattgaacacctgcccaagtacgtcatgatcgcaacaccctccagaaccatcgtctacaccctggtgggcaagcagctgaaggccaccaccgccaccggctgggcctactacgtcaagtccaaggccggcgactacagcaccgaggccaggaccgacaacctctcagagcacgagaagctgctgcacatggtgttcctgggcatcatcgcaggagtggtggtcctggtggtcacagtcgtggtgggagcagtgatctggaggaagaagtgctcaggaaggaagggcccatcctactcccacgccgccagggacgactcaacccagggctcagacagctccctgatggcccccaaggtg

SEQ ID NO: 147 - DNA, 978 bp, Feline Infectious Peritonitis Virus
  source 1..978: mol_type = genomic DNA
atgagattcgtgatgtcccctaccgtactactactcctacttggcgcactagcagctcctcaaacttgggccggatccgagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtc

SEQ ID NO: 148 - DNA, 1128 bp, Feline Infectious Peritonitis Virus
  source 1..1128: mol_type = genomic DNA
atggccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 149 - DNA, 1128 bp, Feline Infectious Peritonitis Virus
  source 1..1128: mol_type = genomic DNA
atggccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 150 - DNA, 1128 bp, Feline Infectious Peritonitis Virus
  source 1..1128: mol_type = genomic DNA
atggcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 151 - DNA, 1098 bp, Feline Infectious Peritonitis Virus
  source 1..1098: mol_type = genomic DNA
atggccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 152 - DNA, 1098 bp, Feline Infectious Peritonitis Virus
  source 1..1098: mol_type = genomic DNA
atggccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 153 - DNA, 1098 bp, Feline Infectious Peritonitis Virus
  source 1..1098: mol_type = genomic DNA
atggcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggtctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 154 - DNA, 1203 bp, Feline Infectious Peritonitis Virus
  source 1..1203: mol_type = genomic DNA
atgagatttgtgatgtcccccactgtcctgctgctgctcctgggagccctggcagccccccagacctgggcaggcagcgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 155 - DNA, 1203 bp, Feline Infectious Peritonitis Virus
  source 1..1203: mol_type = genomic DNA
atgagatttgtgatgagccccactgtgctgctgctgctgctgggagccctggcagccccccagacctgggctggctccgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaac

SEQ ID NO: 156 - DNA, 1203 bp, Feline Infectious Peritonitis Virus
  source 1..1203: mol_type = genomic DNA
atgcgattcgtcatgtcaccaaccgttttactattattactaggagcattagcagcaccgcaaacatgggcaggaagtgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaac

SEQ ID NO: 157 - DNA, 1368 bp, Feline Infectious Peritonitis Virus
  source 1..1368: mol_type = genomic DNA
atgagatttgtgatgtcccccactgtcctgctgctgctcctgggagccctggcagccccccagacctgggcaggcagcgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaacttcctgggcatcatcgcaggagtggtggtgctggtggtgacagtggtggtgggagcagtgatctggagaaagaaatgcagcgggagaaagggccccagctacagccacgccgccagggacgacagcacccagggcagcgacagcagcctcatggcccccaaggtg

SEQ ID NO: 158 - DNA, 1368 bp, Feline Infectious Peritonitis Virus
  source 1..1368: mol_type = genomic DNA
atgagatttgtgatgagccccactgtgctgctgctgctgctgggagccctggcagccccccagacctgggctggctccgccacccagggccagagagtgaactggggcgacgagccctcaaaaaggaggggcagatccaacagcagaggcaggaagaacaacaccatccccctgagcttcttcaaccccatccagctggagccaggctccaagttctggtcagtgtgcccaagggacttcgtgcccaagggcatcggcaacaaggaccagcagatcggctactggaacaggcaggagagatacagaatcgtgaagggccagaggaaggaactgccagaaaggtggttcttctacttcctgggcaccggcccccaggctgacgccaagttcaaagacaagatcgacggggtgttctgggtggccaaggacggcgccatgaacaagccaacaacactgggcaccagaggaaccaacaacgagagcaagccactgaagtttgacggcaagatccccccccagttccagctggaagtcaacaggagcaggaacaacagcaggtccggctcacaaagcaggagcgtgtccagaaacagatcccagtcaagaggaagacagcagtccaacaaccagaacaacgtggaggacaccatagtggccgtgctgcagaagctgggagtcacagagaagcagaggagcagatccaagtccagggacaggggagacagcaagcccagggacaccacacccaacaacgccaacaagcacacctggaagaagacagccggcaagggagatgtgaccaacttctacggcgccagaagcgcctcagccaacttcggagactcagacctggtggccaacggaaacgccgccaagagctacccccagatcgccgaatgtgtcccctcagtgtcctccatgctcttcggctcacagtggtcagcagaggacgacggcgaccaggtgaaggtgaccctgacccacacctactacctgcccaaggacgacgccaagacaagccagttcctggagcagatcgacgcctacaagaggccatcccaggtggccaaggaccagaggcagaggaagagcagaagcaagtcagccgagaagaaaccagaggagctgtcagtcaccctggtggaggcctacaccgacgtgttcgacgacacccaggtggagatgatcgacgaggtgaccaacttcctgggcatcatcgccggcgtggtggtcctggtggtgaccgtggtggtcggagcagtgatctggaggaagaagtgctcaggcaggaagggcccatcctacagccacgccgccagagatgacagcacccaaggctcagacagctccctgatggcccccaaggtg

SEQ ID NO: 159 - DNA, 1368 bp, Feline Infectious Peritonitis Virus
  source 1..1368: mol_type = genomic DNA
atgcgattcgtcatgtcaccaaccgttttactattattactaggagcattagcagcaccgcaaacatgggcaggaagtgcaacacaaggacagagagtaaattggggggatgagcccagcaagaggcgaggcagaagcaactcaagagggagaaaaaacaataccatcccactgtcattcttcaaccccattcaactggagccaggctctaaattctggagtgtatgccccagggactttgtgcccaagggcatagggaacaaggaccagcaaataggatactggaaccggcaggagagatacagaattgtcaagggtcagagaaaggagctgccagagagatggttcttctacttcctaggaacaggcccacaggcagacgctaagttcaaggataagatcgatggtgtcttctgggtcgccaaggatggtgcaatgaataaaccaaccaccctggggaccagggggacaaacaatgagtccaagcccctcaagtttgatggcaaaatccccccacagttccagctggaggtcaacaggagcaggaacaatagccgttcagggtcccagtccagatctgtgtccagaaacaggtcccagagcaggggacggcagcagagtaacaaccagaataatgtggaagacaccatagtagcagtgctccagaaactgggggtcacagaaaaacagaggagcaggtccaagtctagggaccgtggggactctaagccaagggacaccacacccaacaacgccaacaagcacacatggaaaaaaacagcagggaagggtgatgtcaccaacttttacggggccaggtcagcctctgcaaacttcggggatagtgacctggtggccaacggcaatgctgctaaatcctaccctcagattgctgagtgcgtaccctctgtatcctctatgctctttggctcacaatggtctgctgaggatgatggtgaccaggtcaaggtcaccttgacccatacctactatctgcccaaggatgatgcaaaaaccagccagttcctagagcagatagatgcctacaagaggcccagccaggtggccaaggatcagaggcagagaaagagcagatccaagagcgcagaaaagaaaccagaggagttatctgtgaccctggtggaggcctacacagatgtctttgatgatacacaggtggaaatgatagatgaggtgactaacttccttggaatcatagctggggtcgttgtcctcgtagtgactgtagtggtaggcgcagttatctggaggaagaaatgctcggggaggaaagggccctcttacagccatgctgccagggatgactccacacaggggtcagatagcagcctcatggccccaaaggtc

SEQ ID NO: 160 - DNA, 4302 bp, Feline Infectious Peritonitis Virus
  source 1..4302: mol_type = genomic DNA
atgaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 161 - DNA, 4302 bp, Feline Infectious Peritonitis Virus
  source 1..4302: mol_type = genomic DNA
atgaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 162 - DNA, 4302 bp, Feline Infectious Peritonitis Virus
  source 1..4302: mol_type = genomic DNA
atgaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 163 - DNA, 4125 bp, Feline Infectious Peritonitis Virus
  source 1..4125: mol_type = genomic DNA
atgaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccc

SEQ ID NO: 164 - DNA, 4125 bp, Feline Infectious Peritonitis Virus
  source 1..4125: mol_type = genomic DNA
atgaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccc

SEQ ID NO: 165 - DNA, 4125 bp, Feline Infectious Peritonitis Virus
  source 1..4125: mol_type = genomic DNA
atgaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcct

SEQ ID NO: 166 - DNA, 1959 bp, Feline Infectious Peritonitis Virus
  source 1..1959: mol_type = genomic DNA
atgtgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 167 - DNA, 1959 bp, Feline Infectious Peritonitis Virus
  source 1..1959: mol_type = genomic DNA
atgtgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 168 - DNA, 1959 bp, Feline Infectious Peritonitis Virus
  source 1..1959: mol_type = genomic DNA
atgtgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 169 - DNA, 4377 bp, Feline Infectious Peritonitis Virus
  source 1..4377: mol_type = genomic DNA
atgagatttgtgatgtcccccactgtcctgctgctgctcctgggagccctggcagccccccagacctgggcaggcagcaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 170 - DNA, 4377 bp, Feline Infectious Peritonitis Virus
  source 1..4377: mol_type = genomic DNA
atgagatttgtgatgagccccactgtgctgctgctgctgctgggagccctggcagccccccagacctgggctggctcaaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 171 - DNA, 4377 bp, Feline Infectious Peritonitis Virus
  source 1..4377: mol_type = genomic DNA
atgcgttttgtaatgtcacctactgtactactactactactcggagcactagcagcacctcagacttgggccggatcaaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 172 - DNA, 4200 bp, Feline Infectious Peritonitis Virus
  source 1..4200: mol_type = genomic DNA
atgagatttgtgatgtcccccactgtcctgctgctgctcctgggagccctggcagccccccagacctgggcaggcagcaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccc

SEQ ID NO: 173 - DNA, 4200 bp, Feline Infectious Peritonitis Virus
  source 1..4200: mol_type = genomic DNA
atgagatttgtgatgagccccactgtgctgctgctgctgctgggagccctggcagccccccagacctgggctggctcaaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccc

SEQ ID NO: 174 - DNA, 4200 bp, Feline Infectious Peritonitis Virus
  source 1..4200: mol_type = genomic DNA
atgcgttttgtaatgtcacctactgtactactactactactcggagcactagcagcacctcagacttgggccggatcaaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcct

SEQ ID NO: 175 - DNA, 2034 bp, Feline Infectious Peritonitis Virus
  source 1..2034: mol_type = genomic DNA
atgagatttgtgatgtcccccactgtcctgctgctgctcctgggagccctggcagccccccagacctgggcaggcagctgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccac

SEQ ID NO: 176 - DNA, 2034 bp, Feline Infectious Peritonitis Virus
  source 1..2034: mol_type = genomic DNA
atgagatttgtgatgagccccactgtgctgctgctgctgctgggagccctggcagccccccagacctgggctggctcatgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccac

SEQ ID NO: 177 - DNA, 2034 bp, Feline Infectious Peritonitis Virus
  source 1..2034: mol_type = genomic DNA
atgcgttttgtaatgtcacctactgtactactactactactcggagcactagcagcacctcagacttgggccggatcatgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacat

SEQ ID NO: 178 - DNA, 4542 bp, Feline Infectious Peritonitis Virus
  source 1..4542: mol_type = genomic DNA
atgagatttgtgatgtcccccactgtcctgctgctgctcctgggagccctggcagccccccagacctgggcaggcagcaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 179 - DNA, 4542 bp, Feline Infectious Peritonitis Virus
  source 1..4542: mol_type = genomic DNA
atgagatttgtgatgagccccactgtgctgctgctgctgctgggagccctggcagccccccagacctgggctggctcaaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccacttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 180 - DNA, 4542 bp, Feline Infectious Peritonitis Virus
  source 1..4542: mol_type = genomic DNA
atgcgttttgtaatgtcacctactgtactactactactactcggagcactagcagcacctcagacttgggccggatcaaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacatttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 181 - DNA, 4365 bp, Feline Infectious Peritonitis Virus
  source 1..4365: mol_type = genomic DNA
atgagatttgtgatgtcccccactgtcctgctgctgctcctgggagccctggcagccccccagacctgggcaggcagcaccaccaacaatgaatgcatccaggtgaacgtgacccagctggcaggcaatgaaaatttgatcagagacttcctgttcagcaacttcaaggaggagggcagtgtagtggtgggaggctactacccaacagaggtgtggtacaactgcagcagaacagccagaaccacagccttccagtacttcaacaacatccacgccttctactttgtgatggaggccatggaaaacagcacaggaaatgccagaggaaaacccctgctcttccacgtgcacggagagcccgtgtcagtcatcatcagcgcctacagagatgacgtccagcagcggcccctgctgaagcatggactggtctgcatcaccaagaacagacacatcaactacgagcagttcaccagcaaccagtggaacagcacctgcacaggagcagacagaaaaatccccttcagcgtcatccccacagacaacggcaccaaaatctatggcctggagtggaatgatgactttgtgacagcctatatcagcggcaggagctaccacctcaacatcaacaccaactggttcaacaacgtcaccctgctctactccagatccagcacagccacctgggagtacagcgccgcctatgcctaccagggagtctccaacttcacctactacaaactgaacaacaccaacggcctgaaaacctacgagctgtgtgaggactacgagcactgcacaggctatgccacaaatgtgtttgccccaaccagcggaggctacatcccagacggcttctccttcaacaactggttcctcctcaccaactcctccacatttgtgagcggcagatttgtgaccaaccagcccctgctgatcaactgcctgtggcccgtgcccagctttggagtggcagcccaggagttctgcttcgagggagcccagttcagccagtgcaacggagtcagcctgaacaacacagtggacgtgatcagattcaacctgaacttcacagcagacgtgcagagtggaatgggagccaccgtcttcagcctgaacaccacaggaggagtgatcctggagatcagctgctacagcgacacagtgagcgagagcagcagctacagctacggagagatcccatttggcatcacagatggccccaggtactgctacgtcctgtacaatggaacagccctgaaatacctgggcaccctcccacccagcgtgaaggagatcgccatcagcaagtggggccacttctacatcaatggctacaacttcttcagcaccttccccatcggctgcatctccttcaacctgaccacaggagtgagcggggccttctggacaatcgcctacacatcctacacagaagccctggtgcaggtggagaacacagccatcaaaaacgtcacctactgcaacagccacatcaacaacatcaagtgcagccagctgacagccaacctgaacaacggcttctacccagtggccagctcagaggtgggcttcgtgaacaagagcgtggtgctcctgcccagcttcttcacctacacagcagtgaacatcacaattgacctgggcatgaagctgagcggctacggccagccaattgccagcaccctctccaacatcaccctccccatgcaggacaataacacagatgtgtactgcatcagatccaaccagttctctgtctacgtgcacagcacctgcaaaagcagcctgtgggacaacatcttcaaccaggactgcacagatgtcctggaggccacagccgtgatcaaaacaggcacctgccccttcagctttgacaaactcaacaactaccttacattcaacaaattctgcctctccctcagcccagtgggagccaactgcaagtttgatgtggccgccaggaccaggacaaatgaacaagtggtcagaagcctctacgtcatctacgaggagggagacaacatcgtgggggtccccagcgacaacagcggcctgcacgacctgagtgtgctccacctggacagctgcacagactacaacatctacggcaggactggggtgggcatcatcagaagaaccaacagcacactgctgagtggcctgtactacaccagcctgagtggagacttgctgggcttcaagaatgtgtcagatggggtgatctacagtgtgaccccctgtgacgtgtctgcccaggctgcagtcatcgacggagccatcgtgggagccatgaccagcattaacagcgagctgctgggcctgacccactggaccaccacccccaacttctactactactccatctacaactacacatcagaaagaacaagagacacagccatcgacagcaatgacgtggactgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggcccttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 182 - DNA, 4365 bp, Feline Infectious Peritonitis Virus
  source 1..4365: mol_type = genomic DNA
atgagatttgtgatgagccccactgtgctgctgctgctgctgggagccctggcagccccccagacctgggctggctcaaccaccaacaacgagtgcatccaggtgaacgtgacccagctggcaggcaacgagaacctcatcagagacttcctcttctccaacttcaaggaggagggctcagtggtggtcggcggctactacccaacagaggtgtggtacaactgctcaaggaccgccagaaccacagccttccagtacttcaacaacatccacgccttctacttcgtgatggaggccatggagaactccaccgggaacgccaggggcaagccactactcttccacgtgcacggagagccagtgagcgtgatcatctcagcctacagggacgacgtgcagcagcgccccctgctgaagcatggactggtgtgcatcaccaagaacaggcacatcaactacgagcagttcaccagcaaccagtggaacagcacctgcaccggcgcagacaggaagatccccttctcagtgatcccaacagacaacggaaccaaaatctacggcctggagtggaacgacgacttcgtgaccgcctacatcagcggcaggtcctaccatctcaacatcaacaccaactggttcaacaacgtcaccctcctctacagcaggtcatccacagccacctgggagtactcagctgcctatgcataccagggagtctccaacttcacatactacaaactcaacaacaccaacggcctcaagacctacgagctgtgtgaggactacgagcactgcaccggctacgcaacaaacgtcttcgccccaacctccggaggctacatcccagacggcttctccttcaacaactggttcctcctcacaaacagctccaccttcgtgtcaggaaggttcgtgaccaaccagcccctgctcatcaactgcctctggcccgtcccctccttcggagtggccgcccaggagttctgcttcgagggagcccagttctcccagtgcaacggagtctccctcaacaacaccgtggacgtcatcagattcaacctcaacttcacagcagacgtccagagcggcatgggagccaccgtgttcagcctgaacaccacaggaggagtgatcctggagatctcctgctactcagacacagtgtcagagtcctcctcctacagctacggagagatcccattcggcatcacagacggccccagatactgctacgtgctgtacaacggcacagccctgaagtacctgggcaccctccccccatcagtgaaggagatcgccatcagcaagtggggccacttctacatcaacggctacaacttcttctccaccttccccatcggctgcatcagcttcaacctgaccaccggagtgtccggagccttctggaccatcgcctacacatcatacaccgaggccctggtgcaggtggagaacacagccataaagaacgtgacctactgcaacagccacatcaacaacatcaagtgctcccagctgacagccaacctgaacaacggcttctacccagtggcctccagcgaggtgggcttcgtgaacaagagcgtggtcctactcccctccttcttcacctacacagcagtcaacatcacaattgacctgggcatgaagctgtccggctacggccagccaatcgccagcaccctgtccaacatcaccctgccaatgcaggacaacaacaccgacgtctactgcatcagaagcaaccagttctccgtgtacgtccactccacctgcaagtcctccctctgggacaacatcttcaaccaggactgcacagacgtgctggaggccacagctgtgatcaagacaggaacctgccctttctcattcgacaagctcaacaactacctgaccttcaacaagttctgcctgagcctgtccccagtgggagccaactgcaagttcgacgtggccgccagaaccaggaccaacgagcaggtggtcagaagcctgtacgtcatctacgaggagggagacaacatcgtgggagtgcccagcgacaactcaggcctgcacgacctgagcgtgctgcacctggactcctgcacagactacaacatctacggcaggacaggagtgggcatcatcaggaggaccaacagcacactgctgtccggcctctactacacctccctgtccggagacttgctgggattcaagaacgtgtcagacggagtcatctacagcgtcaccccatgtgacgtgagcgcccaggcagcagtgatagacggagccatcgtgggagccatgacctcaatcaactcagaactgctgggcctcacccactggacaacaacacccaacttctactactactccatctacaactacacatcagaaagaacaagggacacagcaatcgactccaacgacgtggactgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggcccttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 183 - DNA, 4365 bp, Feline Infectious Peritonitis Virus
  source 1..4365: mol_type = genomic DNA
atgcgttttgtaatgtcacctactgtactactactactactcggagcactagcagcacctcagacttgggccggatcaaccacaaataacgagtgcattcaggtcaacgtcacccagctggccggtaacgagaacctaattagagacttcctattctcgaactttaaagaggaaggctctgtggtggtcggaggttactaccccacagaagtgtggtacaattgctcacgtacagccaggaccactgccttccagtacttcaacaacattcatgccttctactttgtcatggaagccatggagaactccactgggaatgccagaggaaagcctctcctcttccatgtccatggagagcctgtctctgtgattatctcagcatatagggatgatgtgcagcagcggccgctgcttaagcatggcctagtgtgcattactaagaaccgacatatcaattatgagcagttcacctccaaccagtggaactccacatgcactggtgctgataggaagatcccgttcagcgttatccccaccgataatggcacaaagatttatggcctagaatggaacgatgattttgttactgcctacatatcaggaagaagttaccacttaaacattaacaccaattggttcaataatgttacacttctgtactctcgcagcagtacggccacttgggagtattcggctgcatatgcctaccaaggtgtaagcaacttcacctactacaagctgaacaatacgaacggtctgaagacttatgagctgtgcgaagactacgagcactgtacgggctatgcgacaaatgtcttcgccccgacgagcggcgggtacataccggatggcttctccttcaacaactggttcctccttaccaatagctccactttcgtatcaggaagatttgttacgaaccaaccccttctcattaactgtctgtggccagtgccctccttcggagtagctgctcaagagttctgtttcgagggtgcacagttcagccagtgtaatggagtgtcgctgaacaacactgtggacgtgatcaggtttaatttgaacttcacagctgatgttcagtccggcatgggcgcgactgtgttcagcctaaacaccacgggtggcgtcatcttggagattagttgttactctgacactgtgtcagagagcagcagttactcctacggagaaattcctttcggcatcacagacggtccccggtactgctatgtgctgtacaacggaactgctttgaagtacctggggacattgccaccttctgtgaaggaaatagccatctctaagtggggtcacttttacattaacggctataatttcttttccactttcccaattggatgcattagcttcaacctgacaacaggtgtgtctggagccttctggaccatcgcctatacctcttacacagaggctctagtacaggtggagaacacagctataaagaacgtgacgtactgtaacagtcacataaacaatatcaagtgttctcagttgactgcgaacttaaacaatgggttttatccagtggcgagctcggaggtggggtttgtaaacaaatctgtggtgctgttgccctccttcttcacgtacactgcagtgaacatcaccattgatttggggatgaaactgtccggctacgggcagcctatagcatctacactgagcaatatcacactgcccatgcaggataacaatacagatgtgtactgtatccgctcaaaccagttctctgtatacgtgcacagtacatgcaagagctcgctatgggacaacattttcaaccaggattgtactgatgtgcttgaagcaactgcagtgatcaaaacaggcacatgcccgttcagctttgataagctcaacaactacctaacgttcaacaagttctgcttgagcctgtctccagtaggcgccaattgcaagtttgacgttgcagcgcgaacacggacaaacgaacaggtagtgcggtcgctctatgttatctacgaggagggggacaacatagtcggggttccatccgacaactcaggtttgcacgacctgagtgtgctccatttggactcatgcacggattataacatctacgggcgcacaggtgtggggataatacgaagaacaaactctacgctattgagcgggctctactacacctcattgagtggggacctgctagggttcaagaacgtatctgacggtgtgatctatagcgtcacaccatgtgacgtatcagcccaagctgctgtgattgacggggcgattgtgggggctatgacttcaattaacagcgagctcctaggcctgacccactggactaccaccccaaacttctactactacagcatttataactataccagtgagcgcaccagggacactgccattgacagcaatgacgtcgactgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggcctttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 184 - DNA, 2199 bp, Feline Infectious Peritonitis Virus
  source 1..2199: mol_type = genomic DNA
atgagatttgtgatgtcccccactgtcctgctgctgctcctgggagccctggcagccccccagacctgggcaggcagctgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggacagcatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagatcctggccaacgctttcaaccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaacattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagaactacgaaccaattgaaaaagtgcacgtccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 185 - DNA, 2199 bp, Feline Infectious Peritonitis Virus
  source 1..2199: mol_type = genomic DNA
atgagatttgtgatgagccccactgtgctgctgctgctgctgggagccctggcagccccccagacctgggctggctcatgtgagccagtcatcacctactccaacatcggcgtgtgcaagaacggagccctggtgttcatcaacgtcacccactcagacggcgacgtccagccaatctccacaggaaacgtcaccatccccaccaacttcaccatcagcgtgcaggtggagtacatgcaggtctacaccaccccagtctccatcgactgtgccaggtacgtgtgcaacggcaacccaagatgcaacaaactgctgacccagtacgtgagcgcctgccagaccatcgagcaggccctggccatgggcgccaggctggagaacatggaggtggacagcatgctctttgtgagcgagaacgccctgaagcttgccagcgtggaggccttcaacagcaccgaaaacctggactccatctacaaagagtggccctccataggaggctcctggctgggaggcctgaaggacatcctcccatcccacaacagcaaaagaaagtacggcagcgccatcgaagacctgctgttcgacaaggtggtcacctcaggactgggcacagtggacgaggactacaagaggtgcaccggaggctacgacatcgcagacctggtctgtgcccagtactacaacggcatcatggtgctcccaggcgtggccaacgccgacaagatgaccatgtacacagcaagcctggctggaggaatcacactgggagccctgggaggaggggccgtggccattccattcgccgtggccgtgcaggccagactgaactacgtggccctgcagacagacgtgctaaacaagaaccagcagatcctggccaacgccttcaaccaggccatcggcaacatcacccaggccttcggcaaggtgaacgacgcaatccaccagacatcacagggcctggcaacagtggccaaggccctggccaaggtccaggacgtggtgaacacccagggccaggccctctcacacctgacagtccagctgcagaacaacttccaggcaatctcctcctccatctcagacatctacaacagactggaccccccctcagccgacgcccaggtggacagactcatcacaggcaggctgaccgccctcaacgccttcgtgtcccagaccctcaccaggcaggccgaggtgagggccagcaggcagctcgccaaggacaaggtgaacgagtgcgtcagaagccagagccagaggttcggcttctgtggcaacggcacccacctgttctccctggccaacgcagcccccaacggcatgatcttcttccacacagtcctcctcccaacagcatatgagacagtcaccgcctggtcaggaatctgtgcctcagacggggacagaaccttcggcctggtggtcaaggacgtgcagctgacactcttcagaaacctggacgacaaattctacctgacccccaggaccatgtaccagccaagggtggccacctcctcagacttcgtgcagatcgagggctgtgacgtgctcttcgtgaacgccaccgtcatcgacctcccatccatcatcccagactacatcgacatcaaccagacagtgcaggacatcctggagaactaccgccccaactggaccgtgccagagttcaccctagacatattcaacgccacctacctgaacctgacaggagaaattgacgacctggagttcagatcagaaaagctacacaacaccaccgtggagttagccatcctcatagacaacattaacaacaccctcgtcaacctggagtggctcaacaggattgaaacctacgtgaagtggccctggtacgtctggctcctcatcggcctggtggtggtcttctgcatcccactgctgctgttctgctgcttctccaccggctgctgtggatgcatcggctgcctgggctcatgctgccactcaatctgctcaaggaggcagtttgaaaactacgagccaatagaaaaagtccacgtccacttcctgggcataatcgccggcgtggtggtgctggtggtcacagtggtggtcggagcagtgatctggaggaagaagtgctcagggaggaagggcccatcctactcccacgccgccagggacgacagcacccagggctcagactcatccctgatggcccccaaggtg

SEQ ID NO: 186 - DNA, 2199 bp, Feline Infectious Peritonitis Virus
  source 1..2199: mol_type = genomic DNA
atgcgttttgtaatgtcacctactgtactactactactactcggagcactagcagcacctcagacttgggccggatcatgcgagcctgttattacctacagcaacatcggtgtttgtaagaatggagctctagtcttcataaacgtaacgcactctgatggcgatgttcaaccaatttccactgggaacgtaaccatacccaccaactttactatttccgtccaggtggagtacatgcaagtatataccacgccagtgtccatcgactgcgctcggtatgtgtgcaacggtaacccacgctgcaataagctgctaacgcagtacgtcagcgcctgccagacaatagagcaggcattggcaatgggtgcaaggcttgaaaacatggaggtggactccatgttgttcgtgtctgaaaacgctcttaaactagcatccgtggaggcattcaacagtactgagaacttggactctatctataaggagtggccctccattgggggcagctggcttggaggtctaaaagacatcctgcccagccacaactccaagaggaagtacgggtccgctatagaggacctcctctttgacaaggttgttacttctggtcttggcacagtggacgaagactacaagaggtgcacaggaggctatgatatagctgacctggtgtgtgctcaatactacaacggtataatggttctcccaggtgtggccaacgctgacaagatgacaatgtacacagcctctttagctggaggcattaccctgggagcccttgggggtggcgcagtggcaattccatttgccgttgcggtgcaggcccgactaaactatgtcgcacttcaaacagatgtgctcaacaagaaccaacaaatactggccaacgctttcaaccaggccattggtaacattacgcaggcatttggcaaggtgaatgacgccatccaccagaccagccagggacttgccacagtggccaaggccttggcaaaggtgcaggatgtcgtgaacacacagggtcaggccctctctcatttgacagtgcagcttcagaataacttccaagcaatcagttcaagcatcagcgacatctacaaccggctggaccccccatctgcagatgcgcaggtggacaggctaatcactggacgcttgacggcactaaatgcctttgtcagccaaactctgacccggcaagcagaggtgcgggccagtagacaactggccaaagacaaggtcaacgagtgcgtcaggtcccagtcccagcgttttggattctgtgggaacgggacgcacctgttctcattagccaatgctgcacccaatggcatgatctttttccatactgttctacttcctactgcctatgaaaccgtgaccgcttggagcggcatctgcgcatctgatggcgataggaccttcgggctggtcgttaaggatgtccagctaacgctgttccggaacttggatgacaagttctacctgacccccaggaccatgtaccagccgagagtggcaacgagttctgacttcgtgcaaattgagggctgtgacgtcctgtttgttaatgcaacagtgatcgatctgcccagtatcataccagattacatagacataaaccagacagtccaggacatactggagaattacaggccaaactggaccgtaccagagttcacgctggacatattcaacgctacgtacctcaatttgactggggaaattgatgacttggagttcaggtcggagaagctccacaacaccactgtggagctggccatcctgattgacaacatcaacaacactctggtgaacctggagtggctaaatcgcattgaaacctatgtcaagtggccttggtacgtttggctactgatcggactcgtggtagtcttctgcataccactcctgctattttgctgcttcagcacagggtgctgtggctgcattggatgcctaggttcctgctgtcacagtatctgcagcagaagacaattcgagaactacgagcccatagaaaaggtccacgtacatttcctggggataatcgcaggagtggttgttctagtggtgaccgtggtagttggggcagtgatctggagaaagaaatgctctggccgtaagggaccatcctactcccatgcagcacgtgatgattctacccagggcagcgacagttcattgatggcccctaaagtc

SEQ ID NO: 187 - DNA, 303 bp, Feline Infectious Peritonitis Virus
  source 1..303: mol_type = genomic DNA
atgctggtcttcctgcatgctgtgctggtgactgtgctcatcctgcccctcatcggccgcatccagctgctggagagacttctcctgagccacctgctgaacctgaccacagtcagcaatgtcctgggggtcccagacagcagcctgcgggtcaactgcctgcagctgctgaagccagactgcctggacttcaacatcctgcacaaggtgctggcagaaacacggctgctggtggtggtgctgcgggtcatcttcctggtgctgctgggcttcagctgctacaccctgctgggggccctcttc

SEQ ID NO: 188 - DNA, 180 bp, Feline Infectious Peritonitis Virus
  source 1..180: mol_type = genomic DNA
atggatgctgtgaagagcatcggcatctctgtggatgctgtgctggatgagctggacagcattgcctttgctgtcaccctgaaggtgctcttcaacagcgggaagctgctggtgtgcatcggcttcggggacacctttgaggaggctgagcagaaggcctatgccaagagcaagctggtg

SEQ ID NO: 189 - DNA, 981 bp, Feline Infectious Peritonitis Virus
  source 1..981: mol_type = genomic DNA
atgagatttgtgatgtcccccactgtcctgctgctgctcctgggagccctggcagccccccagacctgggcaggcagcgagagatactgtgccatgaaggacgacagcagcaacacctgcatcaacggcaccaacagcagctgccagacctgctttgagagaggggacctgatctggcacctggccaactggaacttcagctggagcgtgatcctgatcgtgttcatcaccgtgctgcagtatggaagaccccagttcagctggctggtgtacggcatcaagatgctgatcatgtggctgctgtggcccatcgtgctggccctgaccatcttcaacgcctacagcgagtaccaggtgtccagatacgtgatgtttggcttcagcatcgccggggccgtggtgaccttcgccctgtgcatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 190 - DNA, 1131 bp, Feline Infectious Peritonitis Virus
  source 1..1131: mol_type = genomic DNA
atggctacacaaggacagagagttaactggggagatgaaccaagcaagagaagagacagaagcaacagcagaggaagaaaaaatggaaacatccccctgtcctacttcaaccccatcaccctggagagcggcagcaagttctggaacatctgccccagggactttgtgcccaagggcattggaaacaaggaccagcagatcggctactggaacagacaggtgcgctacagaattgtgcggggccagaggaaggagctgcccgagagatggttcttctacttctctggaacaggcccccacgctgatgccaagttcaaggacaagattgatggagtgttctgggtggccagagatggagccatgaacaagcccaccaccctgggcaccagaggcaccaacaacgagagcaagcccctgaagtttgatggcaagatccccccccagttccagctggaggtgaacagaagcagaaacaacagcagaagcggcagccagcccaggagcgtgtccagaagcagaagccagagcagaggaagacagcagagcaacaaccagaacaccaacgtggaggacaccatcgtggccgtgctgagcaagctgggcgtgacagacaagcagaggagcagaagcaagtctggagaaagaaaccagagcaagcccagagacaccacccccaagaatgccaacaagcacacctggaagaagacagctggcaagggggacgtgaccaacttctatggagccaggagcagcagcgccaactttggagacagcgacctggtggccaatggaaatgccgccaagtgctacccccagatcgccgagtgtgtgcccagcgccagcagcatcctgtttggcagccagtggagcgccgaggaggccggggaccaggtgaaggtgaccctgacccacacctactacctgcccaaggatgatgccaagaccagccagttcctggagcagattgatgcctacaagaggcccagcgaggtggccaaggaccagaggcagaggaagagcagaagcaagtctgctgacaagaagccagaggagctgtcggtgaccctcgtcgaggcatacaccgatgtcttcgacgacactcaggtggagatgatcgacgaggtcaccaac

SEQ ID NO: 191 - DNA, 2181 bp, Feline Infectious Peritonitis Virus
  source 1..2181: mol_type = genomic DNA
atgagatttgtgatgtcccccactgtcctgctgctgctcctgggagccctggcagccccccagacctgggcaggcagctgcagcagcgtgatcacctacagcagcttcgccatctgcaacacaggagagatcaagtacgtgaacgtgacccacgtggagactgtggacgacaacatcggggtgatcaagcccatcagcaccggcaacatcaccatccccaagaacttcacagtggccgtgcaggccgagtacatccagatccaggtgaagcctgtggtggtggactgcgccaagtacgtgtgcaatggaaatggccactgcctgaacctgctgacccagtacacctctgcctgccagaccatcgagaacgccctgaacctgggcgccagactggagtccctgatgctgtctgagatggtgaccgtgagcgagagaaacctggacctggccaccgtggagaagttcaacagcacagtgctgggcggggagaagctgggaggcttctactttgacggcctgaagagcctgctgcctcccaccatcggcaagagaagcgccgtggaggacctgctgttcaacaaggtggtgaccagcggcctggggaccgtggatgatgactacaagaagtgcagcgccggcacagatgtggccgacctggcctgtgcccagtactacaacggcatcatggtgctgcctggagtggtggaccagaacaagatggccatgtacaccgccagcctgattggaggcatggccctgggcagcatcaccagcgccgtggccgtgcccttcgccatgcaggtgcaggccagactgaactacgtggccctgcagacagatgtgctgcaggagaaccagaagatcctggccaacgccttcaacaacgccatcggcaacatcaccctggccctggggaaggtgagcaacagcatcaccaccatctctggaggcttccacaccatggccagcgccctgaccaagatccagagcgtggtgaaccagcagggggaggccctgtcccagctgaccagccagctgcagaagaacttccaggccatctcctcttccattgccgagatctacaacagactggagaaggctgaggccgacgcccaggtggacaggctgatcacaggaagactggccgccctgaacgcctacgtgtcccagaccctgacccagtatgccgaggtgaaggccagcagacagctggccatggagaaggtgaatgagtgtgtgaagagccagtctgacagatacggcttctgtggaaatggaacccacctgttcagcctggtgaactctgcccctgacggcctgctgttcttccacaccgtgctgctgcccacagagtgggaggaggtgacagcctggagtggcatctgtgtgaatgacacctacgcctacgtgctgaaagactttgactacagcatcttcagctacaacggcacctacatggtgacccccagaaacatgttccagcccagaaagccccagatgtcagacttcgtgcagatcaccagatgcgaggtgaccttcctgaacacaacctacaccaccttccaggagatcgtgatcgactacatcgacatcaacaagaccatcgccgacatgctggagcagtacaacctgaactacacaacccctgagctgaacctgcagctggagatcttcaaccagaccaagctgaacctgaccgccgagatcgaccagctggagcagagagccgacaacctgaccaacatcgcccacgagctgcagcagtacatcgacaacctgaacaagaccctggtggacctggagtggctgaacagaattgaaacctacgtgaagtggccctggtacgtgtggctgctgatcggcctggtgatcgtgttctgcatccctctgctgctgttctgctgcctgagcaccggctgctgtggctgcttcggcttcctgggctcctgctgccactccctgtgcagcaggaggcagtttgagtcctacgagcccatcgagaaggtgcacatccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 192 - DNA, 978 bp, Feline Infectious Peritonitis Virus
  source 1..978: mol_type = genomic DNA
atgagatttgtgatgtcccccactgtcctgctgctgctcctgggagccctggcagccccccagacctgggcaggcagcgagagatactgtgccatgcaggacagcggcctgcagtgcatcaacggcaccaacagcagatgccagacctgctttgaaagaggggacctgatctggcacctggccaactggaacttctcctggagcgtgatcctgatcgtgttcatcaccgtgctgcagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccgtgcagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggacagacaacctgagcgagcacgagaagctgctgcacatggtgttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 193 - DNA, 1131 bp, Feline Infectious Peritonitis Virus
  source 1..1131: mol_type = genomic DNA
atggctacacaaggacagagagttaactggggagatgaaccaagcaagagaagaggaagaagcaacagcagaggaagaaagaacaatgacatccccctgtccttctacaaccccatcaccctggagcagggcagcaagttctggaacctgtgccccagggacctggtgcccaagggcatcggcaacaaggaccagcagattggctactggaacagacagatcagatacagaattgtgaagggccagaggaaggagctggccgagaggtggttcttctacttcctgggcaccggcccccatgctgatgccaagttcaaggacaagattgatggagtgttctgggtggccagagatggggccatgaacaagcccaccaccctgggcaccaggggcaccaacaatgagagcaagcccctgaggtttgatggcaagatccccccccagttccagctggaggtgaacagaagcagaaacaacagcagaagcggcagccagagcagaagtgtgtccagaaacagaagccagagcagaggaagacaccacagcaacaaccagaacaacaatgtggaggacaccatcgtggccgtgctggagaagctgggggtgacagacaagcagaggagcagaagcaagcccagagaaagaagtgacagcaagcccagggacaccacccccaagaatgccaacaagcacacctggaagaagacagctggaaaaggagatgtgaccaccttctatggagccaggagcagcagcgccaactttggagacagtgacctggtggccaatggaaatgctgccaagtgctacccccagattgctgagtgtgtgccctctgtgagcagcatcatctttggcagccagtggtcagcagaggaggctggggaccaggtgaaggtgaccctgacccacacctactacctgcccaaggatgatgccaagaccagccagttcctggagcagattgatgcctacaagaggcccagcgaggtggccaaggaccagaggcagaggaggagcctgagcaagtctgctgacaagaagccagaggagctgtcggtgaccctcgtcgaggcgtacaccgacgtcttcgacgacactcaggtggagatgatcgacgaggtcaccaac

SEQ ID NO: 194 - DNA, 2199 bp, Feline Infectious Peritonitis Virus
  source 1..2199: mol_type = genomic DNA
atgagatttgtgatgtcccccactgtcctgctgctgctcctgggagccctggcagccccccagacctgggcaggcagctgtgagccagtcatcacctacagcaacatcggagtgtgcaagaacggagccctggtgttcatcaacgtgacccacagcgacggagatgtccagcccatcagcacaggaaatgtgaccatcccaaccaacttcaccatcagcgtccaggtggaatacatgcaggtgtacaccaccccagtgtccatcgactgtgccagatacgtgtgcaatggaaaccccagatgcaacaagctcctcacccagtacgtgtcagcctgccagacaatcgagcaggccctggccatgggagccaggctcgagaacatggaagtggacagcatgctgtttgtctcagagaatgccctgaaactggccagcgtggaggccttcaacagcacagagaacctggaccccatctacaaggagtggccatcaatcggaggcagctggctgggaggacttaaggacatcctgccaagccacaacagcaaaagaaagtacggcagcgccattgaggacctgctgtttgacaaggtggtcacctccggcctgggcacagtggatgaggactacaagagatgcaccggcggctatgacattgccgacctggtgtgtgcccagtactacaatggcatcatggtgctgcctggagtggccaacgccgacaaaatgaccatgtacaccgcctccctggctggaggcatcacactgggagccctggggggaggagcagtggccatcccctttgcagtggctgtgcaggccagactcaactacgtggccctgcagacagacgtgctcaacaagaaccagcagaacctggccaatgccttcatccaggctatcggaaacatcacccaggcctttggaaaagtgaatgatgccatccaccagaccagccagggcctggccacagtggccaaggccctggccaaggtgcaggacgtggtcaacacccagggccaggccctcagtcacctcacagtacagctccagaacaacttccaggcaatctcctcctccatcagcgacatctacaacaggctggaccccccaagcgctgatgcccaggtggacagactgatcacaggaagactcacagccctcaacgcatttgtgtcccagacactgaccaggcaggcagaggtcagggccagcaggcagctggccaaggacaaggtgaatgagtgcgtgaggagccagagccagagatttggcttctgcggaaacggcacccacctgttcagcctggccaacgccgcccccaacggcatgattttcttccacacagtcctcctccccacagcctacgaaacagtgacagcctggtcaggcatctgtgccagcgacggagacagaacctttggcctggtggtgaaggatgtgcagctcaccctcttcagaaacctggatgacaagttctacctcaccccaagaaccatgtaccagcccagagtggccacaagcagcgactttgtgcagattgagggctgtgacgtgctgtttgtgaatgcaacagtgattgacctcccaagcatcatcccagattacatcgacatcaaccagacagtgcaggacatcctggagaactacaggcccaactggacagtgccagagttcaccctggacatcttcaacgccacctacctgaacctgacaggagaaattgacgacctggagttcagatcagaaaaacttcacaacaccaccgtggagcttgccatcctcattgacaccattaacaacacactggtcaacctggaatggctgaacagaattgaaacctacgtgaagtggccctggtatgtgtggctgctgattggactggtggtggtgttctgcatcccactgctgctgttctgctgcttcagcaccggctgctgtggatgcatcggctgcttgggcagctgctgccacagcatctgcagcaggaggcagtttgagtactacgagcccatcgagaaggtgcacgtgcacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 195 - DNA, 984 bp, Feline Infectious Peritonitis Virus
  source 1..984: mol_type = genomic DNA
atgagatttgtgatgtcccccactgtcctgctgctgctcctgggagccctggcagccccccagacctgggcaggcagcgaccgctactgtgccatgcagcacgccagcagcaccagctgcatcaatggcaccagcaccaacagctgccagacctgctttgaaagaggagacttgatttggcacctggccaactggaacttcagctggagcgtcatcctcatcgtgttcatcaccgtgctgcagtatggaagaccccagctgagctggtttgtgtacggcatcaagatgctcatcatgtggctgctgtggcccatcgtgctggccctgaccatcttcaacgcctacagcgagtaccaggtgtccagatacgtgatgtttggcttctctgtggctggagctgtcatcacctttgccctgtggatgatgtactttgtgaggagcatccagctgtacaggaggaccaagagctggtggagcttcaacccagaaaccaatgccatcctgtgtgtgaatgccctgggcaggagctacgtgctgcccctggatggcatccccactggagtcaccctgaccctgctgtctggaaacctgtatgctgagtgcttcaagatggtgggcggcctgaccatcgagcacctgcccaagtacgtgatgattgccacccccagcagcaccatcgtgtacaccctggtgggcaagcagctgaaggccaccacagccaccggctgggcctactatgtgaagagcaaggctggagactacagcacagaggccaggacagacaacctgtctgaacatgagaagctgctgcacatggtgttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 196 - DNA, 1131 bp, Feline Infectious Peritonitis Virus
  source 1..1131: mol_type = genomic DNA
atggccacccagggccagagggtcaactggggcgacgagcccagcaagaggagaggaagaagcaacagcagaggaaggaagaacaatgacatccccctgagcttctacaaccccatcaccctggagactggcagcaagttctggaatgtctgccccagggactttgtgcccaagggcatcggcaacaaggaccagcagattggctactggaacaagcaggcccgctacagaattgtgaagggccagaggaaggacctgcctgagaggtggttcttctacttcctgggcacaggcccccacgctgatgccaagttcaaggacaagattgatggagtcttctgggtggccaaggatggagccatgaacaagcccaccaccctgggcaccagaggcaccaacaatgagagcaagcccctgagatttgatgggaagatccccccccagttccagctggaggtgaaccagagcagaaacaacagcagaagcggcagccagagcagaagtgcctccagaaacagaagccagagcagaggaagacagcagagcaacaaccagaacaccaacgtggaggacaccatcgtggctgtgctgcagaagctgggcgtgacagacaagcagaggagcagaagcaagagcagagaaagaagcggcagcaacagcagggacaccacccccaagaatgccaacaagcacagctggaagaagacagctggcaagggagatgtgaccaacttctatggagccaggagtgccagcgccaacttcggggacagtgacctggtggccaatggaaatgccgccaagtgctacccccagattgctgagtgtgtgcccagcgtgtcctccatgctgtttggaagccagtggtcagcagaggatgctggggaccaggtgaaggtgaccctgacccacacctactacctgcccaaggatgatgccaagaccagccagttcctgggccagattgatgcctacaagaggcccagccaggtggtgaaggagcagaggcagaggaagagcagaagcaagtctgctgacaagaagccagaggagctgtctgtgaccctggtggaggcctacacagacgtgtttgatgacacccaggtggagatgattgatgaggtgaccaac

SEQ ID NO: 197 - DNA, 2199 bp, Feline Infectious Peritonitis Virus
  source 1..2199: mol_type = genomic DNA
atgagatttgtgatgtcccccactgtcctgctgctgctcctgggagccctggcagccccccagacctgggcaggcagctgtgagcccatcatcacctacttcaacatcggggtgtgcaagaatggggccctggtcttcatcaatgtgacccacagcgatggagatgtgcagcccatcagcacaggaaatgtgaccatccccaccaacttcaccatctctgtgcaggtggagtacatccaggtgtacaccacccctgtgtccatcgactgcagccgctacgtgtgcaatggaaaccccaggtgcaacaagctgctgacccagtacttctctgcctgccagaccatcgagcaggccctggccatgggagcccggctggagaacatggaggtggacagcatgctgtttgtgtctgaaaatgccctgaagctggcctctgtggaggccttcaacagcagtgagcacctggaccccatctacaaggagtggcccaacattggaggcagctggctggggggcctgaaggacatcctgcccagccacaacagcaagaggaactacagaagtgccatcgaggacctgctctttgacaaggtggtgacctctgggctgggcaccgtggatgatgactacaagaggtgcacaggaggctatgacattgcagacctggtgtgtgcccagtactaccatggcatcatggtgctgcctggagtggccaatgatgacaagatgaccatgtacacagcctccctggctggaggcatcaccctgggggccctgggcggaggagccgtggccatcccctttgctgtggctgtgcaggccaggctcaactacgtggccctgcagacagatgtgctcaacaagaaccagcagatcctggccaacgccttcaaccaggccattggaaacatcacccaggcctttgggaaggtgaatgatgccatccaccagaccagcaagggcctggccaccgtggccaaggccctggccaaggtgcaggatgtggtcaacacccagggccaggccctgagccacctcactgtccagctgcagaacaacttccaggccatcagcagcagcatctctgacatctacaacaggctggatgagctgtctgctgatgcccaggtggacagactcatcaccgggaggctgacagccctgaatgcctttgtcagccagaccctgaccaggcaggcagaggtgcgggcctcccggcagctggccaaggacaaggtgaatgagtgtgtgaggagccagagccagaggtttggcttctgtggaaatggcacccacctcttctccctggccaatgctgcccccaatggcatgatcttcttccacaccgtgctgctgcccaccgcctatgaaacagtgacagcctggagcggcatctgtgcctctgatggggaccacaccttcggcctggtggtgaaggatgtccagctgaccctcttcagaaacctggatgacaagttctacctgacccccaggaccatgtaccagccccgggtggccaccagcagcgactttgtgcagattgagggctgtgatgccctgtttgtgaatgccactgtcatcgagctgcccagcatcatcccagactacattgacatcaaccagaccgtgcaggacatcctgaagaactacaggcccaactggacagttcctgagctgaccctggacatcttcaacagcacctacctgaacctgacaggagaaatcaatgacctggagttcagaagtgagaagctgcacaacaccacagtggagctggctgtgctgatcgacaacatcaacaacaccctggtcaacctggagtggctgaacagaattgaaacctacgtgaagtggccctggtatgtttggctgctcatcggcctggtgctggtgttctgcatccccctgctcatgttctgctgcctgagcaccggctgctgcggctgcttcggctgcctgggcagctgctgccacagcctgttctccagaagacactttgagaactacgagcccatcgagaaggtgcacatccacttcctgggcatcatcgccggcgtggtggtcctggtggtcacagtggtggtgggagctgtgatctggagaaagaagtgcagcggcaggaagggcccaagctacagccacgctgccagagatgactccacccagggcagcgacagcagcctgatggcccccaaggtg

SEQ ID NO: 198 - DNA, 2280 bp, Feline Infectious Peritonitis Virus
  source 1..2280: mol_type = genomic DNA
atgagattcgtgatgtcccctaccgtactactactcctacttggcgcactagcagctcctcaaacttgggccggatcctacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtcagagccaagagaggcagcggagccaccaacttcagcctgctgaagcaggccggcgacgtggaggagaacccaggacctcaccaccaccaccaccacgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 199 - DNA, 2229 bp, Feline Infectious Peritonitis Virus
  source 1..2229: mol_type = genomic DNA
atgagattcgtgatgtcccctaccgtactactactcctacttggcgcactagcagctcctcaaacttgggccggatcctacccatacgatgttccagactacgcttacccatatgacgtgccagactatgcctacccctacgacgtgcccgactacgcagagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcggaggaggaggaagcggaggaggaggaagcggaggaggaggaagcgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaacttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtc

SEQ ID NO: 200 - DNA, 2181 bp, Feline Infectious Peritonitis Virus
  source 1..2181: mol_type = genomic DNA
atgagattcgtgatgtcccctaccgtactactactcctacttggcgcactagcagctcctcaaacttgggccggatccgagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtcagagccaagagaggcagcggagccaccaacttcagcctgctgaagcaggccggcgacgtggaggagaacccaggacctgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaac

SEQ ID NO: 201 - DNA, 2148 bp, Feline Infectious Peritonitis Virus
  source 1..2148: mol_type = genomic DNA
atgagattcgtgatgtcccctaccgtactactactcctacttggcgcactagcagctcctcaaacttgggccggatccgagaggtactgtgccatgcagaacaccggttcccagtgcatcaacggcacagactcctcctgctccacctgcttcgagagaggaggcctgatctggcacctggcaaactggaacttcagctggagcgtgatcctgatagtgttcataaccgtcctgaagtacggcagaccacagttctcatggcttgtctatggcatcaagatgctgattatgtggctgctttggcctatcgtcctggccctgaccatcttcaacgcctactctgagtaccaggtgtcaaggtatgtcatgttcggcttctcagtggctggagctgtggtgacctttgctctgtggatgatgtacttcgtgaggtccatccagctgtacaggaggacaaagtcatggtggtccttcaacccagaaaccaatgccatcctgtgcgtcaacgcactgggcagaagctacgtcctaccactggacggcactcctacaggagtgaccctgaccctgctgtcaggcaatctgtacgcagaggggttcaagatggccggtggcctgaccatcgagcatctgcctaagtacgtgatgatcgccacccctagcaggacaatcgtgtacaccctggtgggaaagcagctaaaggcgaccacagccacaggctgggcctactacgtgaagtccaaggcaggggactattcaaccgaggccaggaccgacaacctgtcagagcacgagaagctgctgcacatggtcggaggaggaggaagcggaggaggaggaagcggaggaggaggaagcgccacacagggccagagggtgaactggggcgacgagccatccaagaggaggggaaggagcaacagcagaggaaggaagaacaacaccatccccctgtccttcttcaacccaattcagctagagccaggcagcaagttctggtcagtgtgccccagagacttcgtgcccaagggcatcggaaacaaggaccagcagatcggctactggaacagacaggagagatacagaattgtgaaaggccagagaaaggagctgccagagaggtggttcttctacttcctgggcaccggcccacaggcagacgccaagttcaaggacaagatcgatggagtgttctgggtggccaaggacggcgccatgaacaagcccaccacactgggcacaagaggaacaaacaatgagagcaagccactgaagtttgatggcaagatcccaccccagttccagctggaggtgaacaggagcagaaacaacagcagaagcggcagccagagcagaagtgtgagcagaaacagaagccagagcagaggaagacagcagagcaacaaccagaacaacgtggaggacaccatcgtggccgtgctgcagaagctgggggtcacagaaaagcagaggagcagaagcaagagcagggacagaggagacagcaagccaagagacaccacccccaacaacgccaacaagcacacctggaagaagacagccggcaagggagatgtgaccaacttctacggcgccagaagcgccagcgccaacttcggagactcagacctggtggccaatggaaacgcagccaagagctacccccagatcgcagagtgtgtgccctctgtctccagcatgctgtttggcagccagtggagcgccgaggacgacggtgaccaggtgaaggtgaccctgacacacacatactacctgcccaaagatgacgccaagaccagccagttcctggagcagattgatgcctacaagaggcccagccaggtggccaaggaccagagacagaggaagagcaggtccaagagcgccgagaagaagccagaagaattgagtgtcaccctggtggaggcctacacagacgtgtttgatgacacccaggtggagatgattgatgaggtgaccaacttcctgggcatcatcgcaggagtggtggtgctggtggtgaccgtggtggtgggggctgtaatctggaggaagaagtgctcagggagaaagggcccaagctactctcacgccgccagggatgactccacacagggctcagactcctcactgatggctccaaaggtc